SEQUENCE LISTING (98 sequences)

SEQ ID NO: 1 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 2 - protein, 351 aa, Mus musculus
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS

SEQ ID NO: 3 - protein, 351 aa, Rattus norvegicus
MSRLLHAEEWAEVKELGDHHRHPQPHHIPQLTPQPPATLQARDHPVYPAELSLLDSTDPRAWLTPTLQGLCTARAAQYLLHSPELGASEAAAPGDEADGQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSVGEQPPPPAASCKNDHHHLRAAASYEGGAGASAVAGAQPAPGGGPRPTPPGACRTRFSTPASSGGYSVQLDALHFPAFEDRALTAMMAQKDLSPSLPGGILQPVPEDSSKTSPRSHRSDGEFSPHSHYSDSDEAS

SEQ ID NO: 4 - DNA, 1065 bp, Homo sapiens
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagttag

SEQ ID NO: 5 - DNA, 1065 bp, Homo sapiens; identical to SEQ ID NO: 4

SEQ ID NO: 6 - protein, 338 aa, Homo sapiens
MMAMNSKQPFGMHPVLQEPKFSSLHSGSEAMRRVCLPAPQLQGNIFGSFDESLLARAEALAAVDIVSHGKNHPFKPDATYHTMSSVPCTSTSSTVPISHPAALTSHPHHAVHQGLEGDLLEHISPTLSVSGLGAPEHSVMPAQIHPHHLGAMGHLHQAMGMSHPHTVAPHSAMPACLSDVESDPRELEAFAERFKQRRIKLGVTQADVGAALANLKIPGVGSLSQSTICRFESLTLSHNNMIALKPVLQAWLEEAEAAYREKNSKPELFNGSERKRKRTSIAAPEKRSLEAYFAIQPRPSSEKIAAIAEKLDLKKNVVRVWFCNQRQKQKRMKYSAVH

SEQ ID NO: 7 - protein, 338 aa, Mus musculus
MMAMNAKQPFGMHPVLQEPKFSSLHSGSEAMRRVCLPAPQLQGNIFGSFDESLLARAEALAAVDIVSHGKNHPFKPDATYHTMSSVPCTSTSPTVPISHPAALTSHPHHAVHQGLEGDLLEHISPTLSVSGLGAPEHSVMPAQIHPHHLGAMGHLHQAMGMSHPHAVAPHSAMPACLSDVESDPRELEAFAERFKQRRIKLGVTQADVGAALANLKIPGVGSLSQSTICRFESLTLSHNNMIALKPVLQAWLEEAEAAYREKNSKPELFNGSERKRKRTSIAAPEKRSLEAYFAIQPRPSSEKIAAIAEKLDLKKNVVRVWFCNQRQKQKRMKYSAVH

SEQ ID NO: 8 - protein, 338 aa, Rattus norvegicus
MMAMNAKQPFGMHPVLQEPKFSSLHSGSEAMRRVCLPAPQLQGNIFGSFDESLLARAEALAAVDIVSHGKNHPFKPDATYHTMSSVPCTSTSPTVPISHPAALTSHPHHPVHQGLEGDLLEHISPTLSVSGLGAPEHSVMPAQIHPHHLGAMGHLHQAMGMSHPHAVAPHSAMPACLSDVESDPRELEAFAERFKQRRIKLGVTQADVGAALANLKIPGVGSLSQSTICRFESLTLSHNNMIALKPVLQAWLEEAEAAYREKNSKPELFNGSERKRKRTSIAAPEKRSLEAYFAIQPRPSSEKIAAIAEKLDLKKNVVRVWFCNQRQKQKRMKYSAVH

SEQ ID NO: 9 - DNA, 1017 bp, Homo sapiens
atgatggccatgaactccaagcagcctttcggcatgcacccggtgctgcaagaacccaaattctccagtctgcactctggctccgaggccatgcgccgagtctgtctcccagccccgcagctgcagggtaatatatttggaagctttgatgagagcctgctggcacgcgccgaagctctggcggcggtggatatcgtctcccacggcaagaaccatccgttcaagcccgacgccacctaccataccatgagcagcgtgccctgcacgtccacttcgtccaccgtgcccatctcccacccagctgcgctcacctcacaccctcaccacgccgtgcaccagggcctcgaaggcgacctgctggagcacatctcgcccacgctgagtgtgagcggcctgggcgctccggaacactcggtgatgcccgcacagatccatccacaccacctgggcgccatgggccacctgcaccaggccatgggcatgagtcacccgcacaccgtggcccctcatagcgccatgcctgcatgcctcagcgacgtggagtcagacccgcgcgagctggaagccttcgccgagcgcttcaagcagcggcgcatcaagctgggggtgacccaggcggacgtgggcgcggctctggctaatctcaagatccccggcgtgggctcgctgagccaaagcaccatctgcaggttcgagtctctcactctctcgcacaacaacatgatcgctctcaagccggtgctccaggcctggttggaggaggccgaggccgcctaccgagagaagaacagcaagccagagctcttcaacggcagcgaacggaagcgcaaacgcacgtccatcgcggcgccggagaagcgttcactcgaggcctatttcgctatccagccacgtccttcatctgagaagatcgcggccatcgctgagaaactggaccttaaaaagaacgtggtgagagtctggttctgcaaccagagacagaaacagaaacgaatgaagtattcggctgtccactga

SEQ ID NO: 10 - DNA, 1497 bp, Homo sapiens
cgctgagcagcgctcacttggagagcggcaagcaagctagacaagcctgattccatgtcacccgctgccaccctgccaggagcgcgaagatgatggccatgaactccaagcagcctttcggcatgcacccggtgctgcaagaacccaaattctccagtctgcactctggctccgaggccatgcgccgagtctgtctcccagccccgcaggtacgtagtggagcataattaccgctctaaggcacattttttgacaggcactagcttcatgtttttttcatgtcgcccagaacaatcgccgctgtctgaacccctctccttgtctcccccgcgttctctcccggcgcgctctctctctcattcatgtctctgatccacacgtctgttccagcagagccgctgcctccgtattaatttttatgacctgggctttgaggagaggcatctcggttgcttgaaaatgtgttttaatcctgtgttgacagtattccctactgaccgtgctgtgcgccttctcgcttgcagctgcagggtaatatatttggaagctttgatgagagcctgctggcacgcgccgaagctctggcggcggtggatatcgtctcccacggcaagaaccatccgttcaagcccgacgccacctaccataccatgagcagcgtgccctgcacgtccacttcgtccaccgtgcccatctcccacccagctgcgctcacctcacaccctcaccacgccgtgcaccagggcctcgaaggcgacctgctggagcacatctcgcccacgctgagtgtgagcggcctgggcgctccggaacactcggtgatgcccgcacagatccatccacaccacctgggcgccatgggccacctgcaccaggccatgggcatgagtcacccgcacaccgtggcccctcatagcgccatgcctgcatgcctcagcgacgtggagtcagacccgcgcgagctggaagccttcgccgagcgcttcaagcagcggcgcatcaagctgggggtgacccaggcggacgtgggcgcggctctggctaatctcaagatccccggcgtgggctcgctgagccaaagcaccatctgcaggttcgagtctctcactctctcgcacaacaacatgatcgctctcaagccggtgctccaggcctggttggaggaggccgaggccgcctaccgagagaagaacagcaagccagagctcttcaacggcagcgaacggaagcgcaaacgcacgtccatcgcggcgccggagaagcgttcactcgaggcctatttcgctatccagccacgtccttcatctgagaagatcgcggccatcgctgagaaactggaccttaaaaagaacgtggtgagagtctggttctgcaaccagagacagaaacagaaacgaatgaagtattcggctgtccactgattgcggcagggcgcagcgtcgggagccgggagagcctagtgctcatccctcccgggttcgggggatggttatcggg

SEQ ID NO: 11 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 12 - protein, 781 aa, Mus musculus
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDL

SEQ ID NO: 13 - protein, 781 aa, Rattus norvegicus
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGPHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDL

SEQ ID NO: 14 - DNA, 2346 bp, Homo sapiens
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaa

SEQ ID NO: 15 - DNA, 40998 bp, Homo sapiens
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgaggtgcttgggcgctcctttccttatccttccggggctgctcccgcttcctctcggagccaaacttcgtagcaggcgcgcggtccgggcggcgggctgggcgcagccgggaggcctggggttgggagcggggagctcaggtgggggacggtgagggtgggccgcgcccggggcgcggagggcggcggccgggcccgggttccggtcgcgctgcctctctggggccctgggggcatcgcttgcggggagggggcgccgcgggggcgcgtacaggagcccggatggcaggcggggtgggggtgggggtgggggtctgtggtttccgtccggggctctggccttggccgagtttgggggagggacccggtgcctcgggatgcgccgggccctgggtggggggcggggtggggacggggggctccgccttctcagctcttgcggcgagttggggttcgggcgctgaggcagagacgccaccctaagtcccatcagtcctggggatcggaccagtggactttctcttaagatttcctctttcattcttaagaatagaagtgttattattttttttaatgccctggctatgtgagtttgaatcgaagcaactttaaaccttagagcaactaaactctaagtgcagcgggtgcgatgcgtcagtagggtgagcacataaaaaatccatgtcttgcacctgtattttagcgtactatgcaggtgagtgaaagcagtggataatgtactgggagtcttatggatttatggtagtgggtatgagaccctggtgaaataagggggtggaggaaggcgaaggtgatggcttactgtttcttaccaagtgaactgcaggattcagcctctgactcagaccgcttcgagaattttgttcgtagaaataatttaaatttattcaaatagtttgatggcagctaaaattgaattatagagcacgttttcttttcagcggagtgaatttttccttcgctccaaagctggccaaatggaattcaagcattgcaacttctttcagtgttttgtctggagagaggactttgaaccgagacttttcgaagttaagttcctatagcctgcttctgaatctgccaagcttgaaagctttggcagttgggtgtatgtagttgttgccttcgttctcttcccttttggagggagcgttgtctcctactttgtatcttccagacatctgtggtcttccccccacccctcgagtttgtgagtggtgaatgaagaaagactaggctgctggtatgcagaggtcggcaaaaggaaatcgaggagtggttttagtgaaatgagagctttgtatcatgaataatggtggcttaggctagacatcaacttgaagagacggcagcatttcctttcataaagtctaggctaatgtttttcagatcgctaagttgtagtttgtctggaatttaggaagccatttcagtatttgtcacttggtgaacgaacattcaataccttcagatgtcttcgtgttgacttgtattcatcctaagaaatagtaaatatagtctcaagtgttatttatgttatactgctggtttattctctgcttaaattattgacataaatttctactttggaggcttttcgtttgaactaaggctgtgcggaatttattttacttttatatttaaatctttgaaaaatctctgattaaaaaaaaagtacccttaaaggtttgaggatgtcctttcacaccagacaaaatttggttaatttgcgcccaatattcattactttgacctaacctttgttctgaaggccgtgtacaaggacaaggccctgagattattgcaacagtaacttgaaaaactttcagaagtctattctgtaggattaaaggaatgctgagactattcaagtttgaagtcctgggggtggggaaaaataaaaaacctgtgctagaaagcttagtatagcatgtaactttagagtcctgtggagtcctgagtctcccacagaccagaacagtcatttaaaagttttcaggaaaaaccaacttaaaaaaaaataaggtggctaattaaaaaaaaatgaagcatttaacagtgttcaggtttcagagtatggaagaggggttttttaaactgttatctgattatttcttttaccaacatgatatagaaaagtgtatttccagtattaaaatttatcagactgagcttactgttcctgttaatgactggaataaaaattggcataaatgagggtctgtatgcttgttttaataacaccaccaccaagatagaaaacgaggaggcaagtttctccaagggtattttgaaatgtgttagcaaaactattgcagatactcgtttttgttatagggtgaggtggggagaggcgcatgctaagtattgttgaaactagggatgtagagaattaaaagtttgaatataattattttgtagttataagtagcagtgaaattaaatctcctgcaatagactatagaagtatatttagccaaatgaaacttcagtgttattgaaatgaaataatacatctgtcctgttacaagattatttttatttctcttgtggtttcctagcttctgataatcaataattgtagatgagtaggtggtaagttttaagtttgtactttgagcttagtcggaagcatgcttgactgccaacccggggcacaaaggatgaaggcttttagaactggacaaacttctaacaaaaggtatttgcaactcttttgtagtgtgtcatgttgatttgtgacattgtttttgaaaatatgtgttaacttagttttcttgtagccctctttttattggaactgtggtatctattgttgaaactgcttgactgagaacatttttataccataaaagtaaatagtaaacatagcccaggagcggcttctggtttgtccatcgtatgtagccattgcctccttgtactctcattgagaagatactgatttgcagattcagttgtccttctctaacagactatttatgtaatattgcagttgtgattgtgataggtaagtggaccagtcggttaaaataaatactcaggtttcacaaaaggaaaataatatgatttgtgttgatctaaatgagtataggagttaactcctatagtttttcatcacttaaactcaggggaaagttctttatttcctctgtttacttaagaatgctgcttttgtgtttcatgcaagactgagcttgactcagtttgaaacctaggctcatctgttgaggcctgaaccctgctgtccttgaagtatgcatataatttgcttccttcctaaggaaaaataagctcttgaaagataaagtcaatcacattaggaacccatttttagggtttagccacttttttttttttttttttttaactcatgggcatctcttctgttaagagacattccccactctccaagtttccctcaagcctgaagcagcagagtgagtagtgttggagcatgttttcattgcatgcttgggtcatgttgagtgccctccagtggatatagtataatgcttgtgatttttttttttttaattccaaacaagtttatgtgggatatatttaggaatagttctgatgagggagaatcaactaagaaacctttgatttctaaaataattaatatcattactgctaattaaaatacaggcttgagaaaatgtcttctcagccaatatttgcagtagaaaagtcgggaggttttttaaggtcactttgagtaggcagttctgcttaaatatatcataatgataaaccagaatctcagtatagtactttaggaggtaaaagatcataatattcagttatattgatgaattacagcaactgaaattctcagaaaaaaattaatgaaaatgtgaattgtcaatttgtctaaaatcattcacagagtaaaacataagtgctcaacttgattatattaggaaatagatagaaataaaggtaattgagccagtgtatgtgacctaaaatataatgcccttagtgaccatagggttggtctcatttgtacatagtggtgggccatgatgaactgtgttttgccctttgaatttttccttaaaaagctttctctaggctcctatgttcatggtttttctgttagtaatattattttctgaaaatccatgtttcaaatcagaatctaattagcaacaggaatgaagcttattctaaattagtttttggaagttaaacggtcagcatatggaaatttttcagggtttagatttttaaaaatttgtttttcagaatatgttgctggaatgaaaacgttagcgtagggacggaaaatgacacttaccagtgattgctttactttgcctgtggaattcagtgtaattttgtggaaacattggtatatgattttttactacttaagaaatgtattgctatagttagggtttttttttttttaaaggcaagaatgcctcaagtgctttatgtgaatgattatttcaggatggattaaatattcctccatcaaggaccatacttgtaaatcagtgatttccaagttggtgcttagtatttacagcatttactgtctataagcttctgttctgatttttcaagagttttctgagaaatgagagtaggcttaaaagttctttgaaaaattatgtacatacaacttactgaaaaaaattgctaccggggacttaatttgtctcttgaaatgggctacttgccttcattaatgtagcatactacaatttgatgttcaagatatgttactaagaataagatcgctttcagaagccttatataggattggtcttactacattgtagtgggaatggctactcaaatgtctccagggccagttaggtattgggtaaatgggaccatgcagactattaaaaattgaagtgcacatgaagcagccagtcataagcagctccagccactgtgtgggaatatagtttatgttgccagatcatctgatttctttcccctaagtgggaaatccagatcaatgtacatctcttgatttgcaagtgttggtgaacaaaattcatattttaagatgctgtattcagcacaaattaaatacacttatttgctgaatactgccagtttgtccctctgcagtagtaccatttgaagtacagtgttttcataatgattctgtgaaatgactggttctgtgaatgtacataatttagcagataacattgttaaattattaggtttgtatttatttaggcacttgggaaatgccttgtgtcaattgattatagattaggagcttaaaagcaagatttatattatcaacttatttgtgaagactgggaaacccacatttttaaagttaggaattaagatggccaggttcaaggaaaagggggagaagtaactttcttattactcaaccatcttaaatagagttctttaagtgtatttttaagaggtctcaaaacttaatctgaagggacgtcaaatgctggacaaattctgtgtatacaactcaagtcagcccccaattttactggtctttaaatcatgtcctttttaccagaagtttgcatttctaagctaaactattactgttagactagatccaaaacttaaaaacagtttaggtaattaaaaattaattgaatataaacgttttacttaaattaatggcaaatggctttttggccaatttaagtttatgtaggcagttaaatcgattttggttaaatcttttgctgctaacaaggtatttccagattttgaaaagtggggtggcctggtgcctgtagtaccagcactttgggaggctggggagggtggatcacctgaggtcaggagttcgagactagcctggccgacgtggtgaatacaaaaattagccaggcatggtggcaggtgcctgtgatcccagctgcttggaagtctgaagcatgagaattgcttgaacctgggaagcggaggttgcagtgagctgagatcacgccactgcactccagctggggcaacagagcgagactccatctcaagaaagaaaagtggggtgtttagtcttcaaactccgtgtttaagtgactggagtgaaaatgtaaatcataggccggtgttggtttaaaaagcatcatctgaaaataatgctgtagtctgcaattatttttattacgatacgatggtgtaaaatacaagcagatcagtgaaccattcatgaaacattaatcctaaaggcgtctcaccccaagtctatcccacaatctccatgagacttcgtggaaccactgtaaagtttcttgtgtaatatcccagaagtttcctacctctggtatcttttgaacttgttgaaaaggcttttccaccccctctttatgatggtttgaagagtgtgaacatctgaatgatgctggggtgaaactgcttcataacacttccattttctcccctatttatttccatatttttattttttcactaatatccccacggttttacttctgttttagtaattcacatgttgctggactaattctttttaactgacttgtaacagatatgttaaaccgtttaaaacttggggggtatttttaacctactttaagttagttcaagttaatcagtctacatggcatataaaccttatgattaataaatcttaaatgctggtagctgagttggaagccaaagacgtacaaaaaagctgaagtgttaggtttagtgtgataagcttctcttactaacagggttttgtaatagcagaaatagatatatgcatatatatgtgcatatatatagcataccttattggatgtccatataaaaatgtgtaagaagttaaatttactgcaaaatttcttgggagtgcaatttgaagatgatcttaagtggtgatagtagtttgctacactgggggatagttgttgcaaactgctcctaattttcctttactgtgaagtaaactgaacagctgtaatagggattaggaactgtactccctctctctcttttttaagtataattaagtggttttggggtaagggtgtagggagtgagtgtctttgaagttttgcatatactagatgaatgccacatgtataagggaggaacaagggattcttggaaatatttttcaatccaagtaactttggaggcttccaagtggagttcattcccctgtgtaggaaagtgctggggtagacccttaaattcctttctgagccattgaaagaatgtcctcaaacttcgcttatactttatagttcatttagatacaaaagttacaaactgaatgctatttaggaaacgtaatacactgacataccgctctttaaatagattataaatttagtatatcaattttctggcattttgctgaattttattgtttagttttcaagcccaactatcttgttactttgtatatcgtagttgtcccccgttgatcactgtttcctgcttaattgtgctgtcgtttttcctgggtcctgattcagagtgtcagcattctgttccccatagaataagaagaggctagaaagtttacagatgagatatctaggaatgccagaagatcaggggtcaccgttgaggcagagtaattaattatggttaaaatggtgttgctgataagtgggtgctgggaaataattaaaatttgattttttagaagaatacttctcatgcttgaagagcgccctcattatatgctaaagggcctcaggtttttccttattgccattatgctgcagattctattacatttgtctgaaaagatctaagacagaagggctgtttaataccttcccttttctcctgaacttcccctctcctctcccccatcaggagctaagtaggaaccccttcaccttgttaccatcagatttcatcaatggtctgtctttacaatgaaggaagtagtactgcattctgggcagaggccagtcctgaggcatgccttttcaaggacattgttactttagttacactggctcttctgttttaactcttatcccccagactctaatcctgttgctttttttggtccccatctcccacctttcatcatctgaaatccattcattgtaacttctggaactcagtcgttagaaaatcctttatattctcaatcttgtgaatgttcctttctttcttattccagctgtaacctagccttctccccaagaatgctacttcccttgcagctctctcaagtggtgaatttttcccttcttgcacaccttataacactgaactaggaggtgtgtggactaaatgtctgcttttgttccttattgtcacttcttgacctttattttccaaaacttcaagctttgactttcatgtgatcaaattataccacccactgcctgtctttatttcaagcacctgcaaaccttcctgggtcattcacatccttctttgttcacttcattagctcttggctcattgtcactgtctcttatttctgtcataattcttggtgacatcagtatctatgtagagcaatactagtgaagatgtggtctggtaactgttacctgtatgaattaagataaggagttatgccagaatataagtcacctgtgtcactaagtttactgtttagcttactttttttgtagcaagattttgatgaaggacgcaatatgttgatttacagtctggtacaaattttgatgtagaagatgcttccaatatcctggtctcttagttccttgatttcttctccagtgatcttattttctaccctaactcaactacatattcccattgtcatatcctagaatattttgtcttttatctgtaactctgctctcttcccccaatctcatttcaagcatcccactttctaattcctctagtaaatacgtcagttccaacagcccatcaatcccattgggacctacagtttatctatccaagcttttccctgttcctcaccctcacttctatacagctgaagtttcatactgaattataatcactttctcgtatacacgtttaacaatcttgtccctccctggcttcatgcccagtgatctcttgtatctatgaccatgtcctttatcttctcctctgtcactggatgaactgtagccttccaagataaggccactcagttcatttgtacagcagattccatcccctcttgctctcaagaatattactgtggtatctctcttttcttgtctctactggctctttccatgagcaaacatggtattatcccattacaaaaaaaattttttctccgtctctccttccactcaccacctcagtctctgcttctctttcccgcaaaataaccttgaaaaattgctttatgtactcccgttttcttttgaacccctgccagtgaccaccacgttataaatttgtagttgtcatctcacttaatctgttagtagtatttggcaccattgctacagttgcttgaaatgccttttcattggtttccaggccaccatgtctgttagcagcttttcctcttacttcactagcatttccttctttgttttttctgttatctttctgacctctgttggagtggctgaaggtttagtccttgaatctttttttgttgtgcatatttactccagtatcatagctttatacagatggtatttacatctgtttgctaacgatttccaaattggtatccttaaactggtatccagctattttttggtcagcattttggatgtctaagaagcttctcaaactaaactgacctcccggttttccccaaagctgcatcttagtcttttccgaaatgcaattctgtctttccagttacctagcttaaaagcttgcagttcttgactcatctttctctcataccacgtatctgaattctctctgcaaaaaattgtctgttctcccttcagaataaagtcacgtgtcattttatgatggggatacattcagaaatgcgtcattaggagataatcatggttgtgtgaacatcagagtatacatagacaaacctagatggtatagcctactacacatctaggctatatggtgtggccaattactatgatgaatactgtaggtaattgtaacataaaggtaggtatttttatctaaacgtattgaaacatagaaaaagtacagtaaaaaatatggtatcaaaaataaaaaatggtacaactgtataaggcagttgtgatgaatggagcttgcaggatatgttgctctgggtgagtcagcgagtgacgattgagggaacgtgaaagatgtgggacatcactgtacactactgtagactttataaacactgtacacttgggctacactacatttttgtaaggttttaaaagacttttttctataataaaccttaaattactgtcacttttttactttatgaattcttaattttttaaacgttttcactcttgtaataacacgtagcttaaaacatacattgtacagctgtacaaaaattttctttatatctttataagcttttttatatttttaaaattactttttaccttttagcttttttgttgaaaaactaagacatgggccaggcgcggtggctcacgcctgtaatcccagcactttgggaggctgaggcaggcggatcacgaggtcaggagataagagaccatcctggctaacatggtgaaaccccgtctctactaaaaatacaaaaaattagccgggcgtggtggcgggcacctgtagtccgagctacttgggaggctgaggcaggagaatggcgtgaacccaggaggcggagtttgcagtgagccgagatagcgccactgcactccagtctgggcgacagagcggaaactccgtctcaaaaaaaaacaaacaaaaaactaagacatgaacacattagcctaggcctacagagggtcaggatcatcagtatcactgtatttccatctccacatcttgtccttctggaatgtcttcagaggcagtaaacataaatggagctgccacctcctgtgataacagtgccttctggaatacctcttgaaggacctacctgtggctgttttatagttaactttttttttttaagaagtaacagaaggagtacactctaatgataaaaagtatagtaagtacataaacctgtaacaatcattatcattatcaagtgtcatgtactggacataactgtatatgctatactttttttttttgagatggcatctcactctgtcacccaggctggagtgcagtggtgcgaggatagctcactgtaacctcagactcctgggctcaagtgatcctcctacctcagcctcccaagtagctgggactacaccaggcaccccaccatgcctggctaattaaaaaaaattttttgtagagacagggtctcactctgttgccagggctggccttgaattcctggcatcaagtaatcctcccactttggcctcacaaagtgcgaggattacaggtaagagccaccatgtctggcccactgtacttttatacaactgaagcacagtaaacctactgtggtttcgtttacaccagcatcaccacaaacaccatgagtagaacattgtgctgcgacgttaacgatggctacaacatcactaggtgataggaatttttcagctccattataatcttatgagaccactgttgtatgtgcagttcatcatccactgaaatgtccttatgtgatgcatgtcttcatatccaaaaatattaatcatttctcactgaagccatgccatgccatgccatcttttgcctgtattattatttttcagcttttattttagattcagggtgtacatgtgcaggtttgttagaaagagtatatcgtatgatgctgaagtttgggatacagttgaaccagtcacccaggtagtgagcatagtactcaatagataacgttctaacattactcctccttccctccctgttcttgtctctgtctattgtatctttatgtccatgtgtaccaaatgtttagctcattcttgtgagaacatgtggcatttgattttgtttctgtgttaatttgcttacaataaatagtctccagctgcatccacattgctacaaaggacatgattttgttcttttttataggctgcatcatattccatggtgtataggtaccacattttcttgatccagtctaccgttcatgggcatttgggttgattgtatctttgctattatggatggcttttgcctatattattggaaaggccttctaactggtgtccctgcttacaccgttttcccccttaaatgtgttttcaacatggtagccagagtaaccctttttataacaataaatcgtgtaacttttttgttcagaaacttacagggcttaccatttcattcagtaaaagctcaagctcctgtatagtcagaccatatccttcatcacctgttacttttctcctctgactcttcagcctttttgtttttcctcaaactgatgaagccttcatggctgatgtcagatgttttgcccattgagatcttccttgttgactcagttgcacttggtcatatgattttcatttatttggggtatctaatcataatctgaaagttggctacttatttttacccctttgagggtccttgccctgtttttgtatccctgatagcgggacagccagatatctggaacttacaggtgttcaataaagttttgttgaatgaatattctggaatcacccaaccttttttttcccctccacttatttttcttctccctttcacggcctgaaagatgtcctatgtatatggttccacttatcactctcatcccagtttgtgatatactattccattatattactattattaatacaattccattgaacttgctcttgctgacttcaccactggacctacatgttggccaaatggatactttataattttagtcttgacccctgcctttggcacatttcttacctctagcacagcactgtccagtaatccacactttctgagacagtggaaatgttcagtatctgtgctgttcagttggtagcaaccagctacccatgcctattaaacatttgaaatgtggctgtgtgactagtggcaattatgttggagagtacagttttagaaactcctgtttttcttacatggcactacatttagtatcacaatctaattgtgcaagccagataggtaggagtcatctttattcctgttatttaatttttctcatctactatatccagttcatcacatcaacagcgcctgttgtttctacctcctaaatatttctttagtctaactactacttgtccctagtgccaccaccatctatcagctggaatattgctatagctgccttacaggtttcccttctttcctgttctcttctagttttttgaattttagtcagcacgagattttaaaaactcaaataagattgtgttattcacctgcttaaaacctttcatgactttcagtgtcacgtagaacagaaaacacttttcttaccaaaggctagagagctctacgtgatctggctatttttaacgtttcattgcactcacccttttcctctataatcaaactactctgatctcaagggttagttcttgaaagatgatcatgttctttaatgactttaggtttttgtgtgttattttctatttctgggatgtttattctctgttccttacatgctggcccttttgcatccttcttcaggtctcagcttacatgttaccttcaagaagcctttgaccactctaagtgggcccttccttccacttctgctgtgtaatcccactcccttctcccacttgttaattagttacatacttttttgtaattgtttatttggttgctgtctccctctcaagaatgcagggaccatgtctgcattctgcagtaatcactactgcacacccagaatctattacagatcctggcatgtagctgatgcataaatatttgttgaatgaaagtctgtacattgtatttatgctattggtattgctatgacctgaaactaaaaggagttgtggaaaagatttcttatggaacagaaatatcccttttgattaatatcacaatctcgtaaattgagaaaacaaaaaaatatatactactggagcattcatgtatagttggagattatgactcatttattggtgtgtttttggactcagaacaaagatgagggaatattccttaaagctctgtattgaaataacgaaaagcagtcacattttaataatagaagcttcctagcttactctttctgtaatcttcttttcctaaatgtaagagagcctcataattatgaggcttattactagagtaaggctgtcaaaggcagcaaaatgtctttctgtttggaagaataacataaacttgacatgtatggtgggggacagaaggtttcaaaagtttaagaatctgtgttgtcttaacaaatagatgcttctcaaggagcttacgctagtggttactctgtccagtcagggttttttcttctttaacttgggttcatttcctgatggcacacatgaagtttggatcatatggtttgactttagctatggtccttagctatggggagcagcatcagcgacctgtgacatgtaaattaaaaatacaatgccagggcccttccccagcccctctgatagagaacctcttggccatctgtatttttagatgttccaggttagtctgattaacacccttggttaagaaccattgggaggatctgattgccagtttaaggggaccttcaagcctgtaggtctttatagttaaaaaaaaaaaaagattttaaaaatcatgcatatgttgtggctgaattctggtttagcacatactgcttttaatggcctgaaatgtttttcccaaataaattgtcttgttatagctttcatgtgtgatttggtccagcttcttgttttgaagatacttacgggggggaacactttgtgatttctcttagtaacatattaacccacttaaaaaccctttctattacaggtcttcacatttaggcttaatgtgcttaattcaaatgtaaaaatacacctgcctttgttctcagtgaaagtatgtaataaataaatgaggggttggcaaactactgcccaccatctgtttttttatggcctatgaactaagaatcgttttggatagctaaaaaaaaaaatcaaaaggataattattttgtgacgtgaaaattatatgaaattcaaatttcagtttctgtgaatgaagttttaatggaacacagccatccatgcttatgtaagtgtgcatattctctggctgttttcactgcaatagcagagttgagtagttgtgacaaagagtttatggcccacaaaacctaaaatatttactttctgatgctttacagaaaaagtttcctgaaccttattctagctatatgttgttcataaatgaatctttcgtggttctgaaggcatttaagaatctcttaggttataaattggctgggcgcagtggctcacgcctgtaatcccagcactttgggaggccgaggctggtggatcacgagggcaggagttcaagatcagcctagccaagatggtgaaaccctgtctccattaaaaaaaaaaaaaaaaaaaaaaaaatagctggggttggtggtgggcagtaatcccagctactcgggaggctgaggcagagaattgcttaaacccaggaggcggaggatgcagtgagccaagatcgcgccactgcactccagcctgggcaacagagtgaaacaccatctcaaaaaaaaaaaaaaaaaaaaaacactcttaggttataaataattgttgttagctctccaagcctccatattacattttgtgtgttctcctgttcacattttgagcattttattttttattagcacattcagttcatcaggtatttaagagcttaatatatgccaaagcatatattaagcgagaagctgtttctaaatgtactgtctcagccctcacagagttcacttcattaggctctttaaaatttctttctttaaaaggtcagcgtgctggtatagtggggaagggaaactcttacaacacgtcgagtagaggaaggttatcattatgggatataatttggaagtcattgagtacctgccattaattctgcctgtagtctgaatgtagagattaacatgtagaaacttttttgaaataaaatcttcaatttctttggcatatctagtactgtctagctaggcatatagtcaaagtatggtgtatatttcaagtattaaaagtttttttgggctgtagtcactgttgaaaggatatagttctttactattacatgtgatacctttatataaaattggctaacccctgtctttcatttatctgcaacactgactgttaccagttgtctctaactttggtatggggggtggaaatatgattagattgaaagggtacatgactgagccacaagcagacctggatttgaattttaactgaacggtttattagctattcttacattaatactgctaatcagttttcttgtgatatgaggaatgatgtcttctttatgaggttgctaggaagattcaatgagataacatactaggctcagaactgaagttgctaggaatttaattatgctaccttgttaaagtatgtcaaaggcagaattcagtgtttagctgataccacaaggcagtatcctaaaattatgctgtaaaagatataaagatgctgtaagtgactcagaaacctagtgactttgtaatgcagttgattcttagaatactgtcactttaacagaataggagctaggaatgaagaaatagttattaaattactaaaatagaaaatttattgacacatgtaaagtgacatttgcttaaatattgaaaaatttgtagtactatttccttgctttagaaaacattggttaccactttttttatttatagcagtttgtttttgccttgaggcaagatggttgactgagtagttgccacatttcttttgtacaaagtccatttcataggccatctagcttttatgcttagaaacatttccttaacgttatatttcagtatttggctaacctatatagggttaaattatataggctaacttctcggacagatatttctaataatttatgtatttggttctgcaaatgtatgcaaaaatatatgtacaaaggtatgcagatgccttgcatacttgatatatgttaaattttttttaatgtagacctttttcgttctctttaatgactatatggtattccaccatcccccgctcacctggacaactacagtaacctcctaaatggtgtttctactttgctattgccccttattgtcttttttcccctttatagctgctggagtgaattttagaaagcctaagtcatacatcacattgcttcatgggcatcccagtacactttggattttattttacatccttactgatctgattctcatctctgtctcttcatggttctctgccttctagttacactggtgacctttcaaaacctttaccacattgagttcattccttacttttcactctttctctgcctggagtgttctgccccatctttacgtggccagctgctcctcctctgatgaaatgtctcttcctcacaggccttccctgaccacccactagagtagcacatcttctacctcataaacttgtttattagtatttcttactctaaattttcttttaaattgcttaattccctaacagtagaatataagcttcactgtatgtatgatcttgttgactctcttactcattgttattgtaataccagtaacaaagggtgtttaaaatttgttcagtgggtgaatatatgttccatttaatggataaattattttttattcagtctcctgttgatggacatttgaataatttccatctttttctctatgaatgcctcacttggcatgcttctgacagtattgccacagaatacatttctgttataaaaattgaatttttaagtcaaagggtagttacactttaatggatagtggcagcttactatcaaaagtttctgctagtttcaccatatccttattagcagtagatattatcaatcttttcaatctttgccaatctgataagcaaaaagtaaatgggtttaaacatcctttgtatatattcattgctcactttatgtttttcctttgaaatgttatttcttgttctttccctgcagtatgattctttctttttttgacttgttcccagttttttgtgtactatggatattagcctttaattatgttacggatgttctagtatgttattttttgaattacttcaaatgtgatttgttgctcagattttaaaaactacatacacaaattatctcatgtttccctttttggtttcaatttcgactcatgcttaatcagttcatcgattgggcatggttttattcttaatatatacccgtattttatctcattttatttttttacgtgtaaatatttggtgaatataggtttaattttaatgtaaaataaggatgaaaaatgatagttggaattacaagcccatttctcctaatacttttaatcaagtaatccactaattgaaatattaccttcttcatttatgaaattgccacattatatctgggtgtttttctgcctactacagtctcttacccatttctttcctaataatacaatacttgaattgctgtggttgttgatttataatgttatcttaatgataacattataaatgtgatggaactggttcctccttatagttcttcttaaatcaagaacaagacatatcttcccatttactctcgtatgtatctcattttactgttatgaatgaaatctgtcctatttgtgtataggaaaatagtttttgtatgtaattgtgatatggccagttttattaaaaatttggttaaactaagagttgttttctgttcagccttatcatactataaaatccacataaaatgggtataaaagtgtcgcaggacactgggctcagatgattctcccacctcagcttcccaagtagctgggactacagcggcatatgccaccacacccagccaatttttaaataagttttaaaaatagtatttttagtagagacagggtttcaccatgttgcccaggctggtcttgaactcctggactcagacaatccacctgccttggcttcccaaagtgttgggattacaggtgtgagccaccacaccttgccgaattgcagccatatttaatacttttttccatcctattccctttgctgcccccaggcctcctgtattgatagcccgctattaagaagctagtgtatattctttgcatacttttacttcataaactatatgaagcattgttctgttttttaacttaattggtataaaattatattttggaaattcagtatattctgtgaaaattatttagaaaatgtgcctctgagataaagcctattcaggatgtatcttaaaggagatagctgtgctttaacattatcagtctttttggctgcttatgttaatataagttggagaaaaacagtctgctttttgtgataatatgttcttggagatggagtgaaagattgtttaaaaacattgtcttttttttcccctgaagtaccagtatttattttaggattatgttactgatcaaagatgctgtgtggagttactcattggtgagactaacaataaatcacacatgcaaaggatgttaccataatctaattattttaaacagtaaaattatattctaagacatccagttggcctatatgtgctatatcaatgactatcaaggggctttttatgtatactgtatacatgtacttcacaaaaatataaaaggatgacatcaaaaatctggcaagccaaaagcctacattacatgtagcaaataaataagcatatgaacttattggaatttaaaaccctgtaggatgggcgggtgatggtatgtatgttagatgtgtggacatatctattaaaagttgtgtcagataacagctggtgctgacaagcccttggtaagatggcagcatgttcaatatgttctgtgaaaattatctcagtttatgatctgtcagtattgtggagctatgcatgaaaggacttaaaattcttacccttaaactcagtaacagtgtttctagaacttctggtgatatgggaaattaagagaattatttatatgcaaaggtgtttattgcagcattgttggaataatagacaaaatggggaagaacaagctcagaatggaggaggtagcttatagtatagacatacgatacaatccagatgataatattttataatagtcttcacaaggaattttatatttttatttttaaaaatacatagcagtgagtttaatataccaaacataccaaaatgtcatcatttactgtgtggtggactcatatgatggagatgataaataaaaatattaatttatttgaggcatatatttatggctgaggaaggaagacagttatgaagaacagctcattctggaaacatactaatttttcccagccataaagagatttcctatttcttttttttttccatttaccttctgtttcctacctgagaagatttcatacttctaataaccatttgtgtacctatttaaagacagtaccaaaggcatacattttagtgtttggaggaccaagggtcatttgatgtttgatgcttattgactattcgaggatgacaagacaccttgagaacacacacacccacacccacacccacaccctcacccacccaccccacccccctccccgaagaaagctgtgaaggaagaaagcagaaaagaacctggagtgagttgtaacttaaaatgttagtgttgcatgaagtgtgttaaaacaggaagatttgaggaaattgcatacattttctagatggcaaagtattactggtgacagttaatgaaaatgcatatgcatgtgtttttagatttacaaattttactaagaactttttaaaaatccctgaaggtgtatcaaaagtttatcatgcttatgaaatagagtagcactttctaactttaaaacggggaataattctttggatcttgattattggaaaagtgaattatgaattgctagtataaaactgtggttttaaaatatgtctgctttatatttttatgtagcagatttactcctagttaataatactcaaacttactgaaaactaaggtaattaagataattctgtcctgatgggaagaggaaaaataacttcagtgtgaaatctattatatattagttgtggcaagatttctcccattgactttgactggagacatttatagggttaaaatcggaaatagcacggtgaattttgaagtatccttgtagttggaaagagtattatgttcatattgccaaaaaaaagatgcatggatgcattagactggatggaaaatacatgagaagttggctagccccctctttgtcaaaacatcacttggtggtgataaagctgttggaaaacacagcattctaatgtagtctgtagtttaatgataatctgtgtcttgaaacatttagcgtagtacttatacaaacctagatggcatagtgtactgcatgcctagcctatatagtatagcctgttgcttctagggtgtaaagctgtatagcgtgttactataggcagttgaaacagtggtatttatgtatccttttttttttttttaaattcttttaagagacagggtcttgctctgttgcccaggctggatgcattggtgtgatcatagctcactataaccttgaactcctaagtgatcctctttgcctcagcctccccagtggctaggactacaggcacatactaccacacctggctaatttttaacatttttttgtagagatggaatttcgctgtgttgtccaggctggtcttggaactcttgtgctgcagcaatccacccgcctcccaaagtgttagaattacaagccacttcgcctggcttgtttacctaaacatagaaaagatccagtaaaaatacagaattaaaatcttgtggggccactgtagcatatgtagtccatcttgactgaaatgtccttatgcagtgcatgattgtacttcataatttttaagcactcctccctcttgattggtacttagtggattttatcatttttgtttcttcataattctttctgaaatgtctactggttggacctttgatctcctgaattgatcgtgatttcttctgttgtattttttgtctttgtcatttttttgtactctaggcagttttctcaattttagtttctattcaactttttgtttttatttattctctccagtatttatggagatactaaattgaagtgttctgtttctctctccaccctatccctagtttcaagttttatctcagtttctatggagtcagttttttcgttgctttaaaaaaaaattttcctgaagtgattggtaagttttggctaattgggagcactagaattgggcccttaatggttggcagggtgtggtggaggagagacagcccttagtccaaaggctcaggccagaaaaagaaagaggaaggctttccttttcctttccggagcagggttctgccctaggtcttgcttggcagtctatttgatttctttagcagttaatgctcagttttttggcatatgtggatctgcctccagagcaggtacaaggtgagtgagtctatgctgttacctaattagatccccatttctaccctttgtttttacttctctatctactgataggtttttaccctccttcacctcatagggttgcagtgaagagcaagatgaatttttatttatgttgcataaattttaaaagctaaaaaatatatatgtaatgttgggaagtcccagtgtacaaatggctattgtaaatttggaacatgaacttgcttttttccattgtaaaaatgaaatcattataaattgcggtcaagttactaggtcagcccacacagagtttacccagtaatatgcgtaaatgttttgcctttgcatcaacaacaaggaaaaacagtactataaaaaaatgttcctggaagccggatgtatcaaagcacttctgaaatagctatatagcctatagacatgaccagttggtttctgagtctgttgacattggccaaaggagaagctcagtgtagaacatgtttggagtctccttttgcagaaatacattggaggctggagtggggaaccaatttttcagaaaggtggtgaagtagttacatagccactcttttaaagacagtcaaaagatagaaactaaggccaggtgttggctcacatctgagataggaaaatcacttgaacctgggaggcggaggttgcagtgagcccagtatgcacctctgcactccagcctggtttggcaagagaccaaaactctgtctcaaaaaaaaacaaaacatagttcacacttaaatattttattccatatctttacatacccaatatgttaatttatagttcaagatgaacttgtttgggacagattttgtaataaaggaaatcgtgttattagaaatatctagaggccatgagcccttaaactgttctaatttgcaagtagttccctgtgtgatgcagtttttttcaatattgcacaataaaggcaaaatacggacaaattagatgataagatttatataaatttttaaaatattgatcaaaatatgtatccatattggtaatatttgtatttataataaatcattgctgtaaatttgaacttagaaaaattttactaataaaggtgcttttgtgttgcaaactttcatttgaaaagtaatttttctttgtaccaaaaaatctaaaattcgctattctagtcaccaaaatttgctttatgaaaaataatttttgatggcactatatcagaaaacaacttgttaaagaaaatgtggagtttttaaaatcccactgtacctctgttatccaaaggggatctgtgaatttttctgtgaaaggttaaaaaaggagagacctttaggaattcagagagcagctgatttttgaatagtgttttcccctccctggcttttattattacaactctgtgctttttcatcaccatcctgaatatctataattaatatttatactattaataaaaagacatttttggtaaggaggagttttcactgaagttcagcagtgatggagctgtggttgaggtgtctggaggagaccatgaggtctgcgtttcactaacctggtaaaagaggatatgggttttttttgtgggtgtaatagtgacatttaacaggtatcccagtgacttaggagtattaatcaagctaaatttaaatcctaatgacttttgattaactttttttagggtatttgaagtataccatacaactgttttgaaaatccagcgtggacaatggctactcaaggtttgtgtcattaaatctttagttactgaattggggctctgcttcgttgccattaagccagtctggctgagatccccctgctttcctctctccctgcttacttgtcaggctaccttttgctccattttctgctcactcctcctaatggcttggtgaaatagcaaacaagccaccagcaggaatctagtctggatgactgcttctggagcctggatgcagtaccattcttccactgattcagtgagtaactgttaggtggttccctaagggattaggtatttcatcactgagctaaccctggctatcattctgcttttcttggctgtctttcagatttgactttatttctaaaaatatttcaatgggtcatatcacagattctttttttttaaattaaagtaacatttccaatctactaatgctaatactgtttcgtatttatagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctggtaagagtattatttttcattgccttactgaaagtcagaatgcagttttgagaactaaaaagttagtgtataatagtttaaataaaatgttgtggtgaagaaaagagagtaatagcaatgtcacttttaccatttaggatagcaaatacttaggtaaatgctgaactgtggatagtgagtgttgaattaaccttttccagatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtaagcaatgacatagctagctttttagtctgctttgaagtaaatgctcaaggggagtagtttcagaatgtctacccaataccagtacttgaaaactaacgatgtttctgaattcctgtattacaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttgggtaagaaaacatgtcagaatgcttgaagctaaaaagtagaagagtatactcacaatatttctgatgaggcttttttcttcttcccagttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaaggtaagagaattattctttatgtggttttcatggagcattggacacctccagtgtcatgtcattccatgcagtgttcctaacctttttggcaccagggaccagtttcgtggaaaacagtttttccatgaatgggttgtgggaatggtttctggatgacaccattccacctcagataatcaggcattagattctcatagggagcgtgcagcctagatccctcgcatgtgcagtccacactagggtttctactcctatgagactctcatggtgcagttgatctgacaggaggtagagctcaagccaggtaatgctcgctcacctgccacttacctcctgctgtgcagcccagttcatttctgttcttttaaatttttgagtttccatatgtaaagcactatgcgaagtagtagggatatggtaggcaagcttctcttcacacttttgttcttaggtgggatgtagatgttgggaataataacctaatatttaatttgtgtagtgggaagaagtggggctatgagggcacataacacaagttgaaactgactctttttgagggttcaaggagacctcttggaggaagtgatagttgagttcagtgttcaaggatgagaagggattcactaggtgaaggttaggtgagaaaacaacatctttgaaacgaaggaaggagatggaaagttttgggaatttaagaaatactaatagtaaggaggaagaaaggtttgaggtgaggctattgagatagacttagcagatctcatagggctttgtagagcatgtttaaaagcacaatgggaaatttcagcagaagcctgaaatgatgaaatttgtttttagaaaattggggcagtgttgaaagggaagatatacagggaatgaaaggacaagcatgaatgatcattttatggtatctgtttttaaggtggatataattaggaaaattaaagggccaaatgatgaggagttaagtgccagttctggttcaaattttcagtgaatcagttttgatataactttcatcttagggcattactcttgcctaccaacatagtttctaaatttttttcttttggtgtgatcactgtgggaagaaggaaattgggcccaaactgatacattgtttggaggactgggatgtctgaatttgagtggaatgctttaaaaggacaagttggatagggccccagtatgggggtctgagtgatggggtccaggaatacatttaggtccaatggcaagctggctgaaattcttgtataataaaataggttggtaatatggctcttctcagacatgtgatcaagattccttgactaacaagatatatatatatatctttctagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtaagtatatgtatctattctgagtcttgtgtatagcatctgcagttctaattagattacttttcttaggaaaaggtggtagaactttaactactgaaaataaatggtcctattcagtttgcagccaagatttacattcagagtacctgtcatctggattgtagctaaatatttaaggctagtttaggtagagttcttattatccatcaaaaatgatggcatatgttttgcttaataaaatttgtttgtaatttcagttttgagtaaacctaagatttgctaacagagctgtgaatttataggagaaaagacaaattctaatatagtacagttttatgtaaagtgattgctttattagtagatgctcatgagcagtttttgttttgttttaacttttaggttccgggtaatgtgcaggcttgttatataggtaaattgcatgtcacaggggtttcgtgtgcagattattttgtcacccaggcagtaagtattgtacccaataggtagtttttcagttctttacctcccacccgtaagtaggccccagtgtctgttgttcccttctttgtgcccgtgtgtactcagtgtttacctcccacttataagtgagaacatgtggtatttggttttctattcctatgttagtttgcttaggataatggcctccagctccatccatgttgctgaggaagacatcttggtatttttttatggctgcttagtattccatagtatatatgtaccacattttctttatctagtctaccattgatgggcatttaggttaattccatatctttgctattgtgaataatgctgcagtgaacatatgcatgcatgtgtctttatggtaaaaagatttctttttctttgggcatatacctaataataggattgctggattgaatggtaattctgtcaggttttttgagaaatcaccaaattgctttccacaatggctgaactaatttactttcccaccagcagtgtataagcattctcttttctcagcaacctcaccagcatctgtcattttttgactttttattagtagccattctaactggtgtgagacggtatctcattgtggttttgatttgcatttctctaatgatcagtgatgtcgagcttttcttcatatgtttcttggccacttgtatgtcttcttttgaaaagtgtctgttcatgtcctttgcccactttttaatggggttgttcttttttgcttgttaatttaagtttattgtaaactctggatattagacctttgtcagatgcatagtttgccagtactttctcccatgccagtactttctcccattctgtaggttgtctgtttactctgttgatttcttttgctgcgcagaagctctttatactgtcccatttgtcagtttttgtttttgttgcaacttctcttggcatcttcgtcatgaaatctttgccaggtcttatgtccagaatggtatttcctaggttatcttgcagagtttttacagttttaagttttatatttaagtctttaatccattctgagttgatttttgtacatcatgtaaggatggggtgcagtttcaatcttggatgtggctagccagttatcccagcaccatttattgaatagggagtcctttccccattgcttgtttttgtttacttgttaggtgtgcggcctaacttctgggctttcttttctgttccattggtctctgtgtctgtttgtataccagtaccatgctgtgattgtaaccttgtattaacagtatagcttgaagttgggtaaagtgattcctccagttttgttctttttgcttaggattgccttggctattcaggctcttttttgggttcatatgaatttttaaatagtttttttttaattatgtgaagaatgccattggtagtttggtaggaatagcattgaatctgtgaattgctttgggctgtatggccattttaacaatattgattcttcctgccatgaaatagaatgttttttcatttgttggtgtcatctctgatttctttgagcagtgttttttgtaattctcattgtagagatctttcacctccctggttagttgtattcctaggtattttattctttttgtggctttggtaaatgggattgcattcttgatttggcttgcagcttggatgttgttggtgtctagaaatgcttctgacttttgtacattgatttttatatcctgaaactttgctgaagtttattggatcaaggagcttttgggcagagattatggggttttctaggtatagaatcatattgtttgcaaacagacttcctatttggatgcattttctttctcttgcctgattatgagcagtgttttgccctgatattctgtattctcagtgaatagatgtcgtctaagtatgagaaacaatttttttctattctgagtatttttaagaaggcaacttatatgtggtactttgtatattgtgtatgttggcaattggggaaaagaatagatggtttgtactagggcctcttgggttctgtgtgtgtgtgtgtgtgtgtgtgtgtgtgtcatgaaaacagttactttttagctaccaagcattttttctcctttcagtaacccacctaacaacatttactcagaatttcaaagcaagcttcaaatcagtattgaaagaaggaaaaatataaaggcatttaatggaagaaaatgttgggaataaagtatagggctggcaacacttacttttctcacttattgagagtaattttacttgggaatttatgagagagaaagacattatgattgctccaggtaactactggcagaggaaccatagtcttggggatagacaaatgtggctgagttcatatagaatgaggggatgggatgtaaattctgtcagctgttccagcagtaacctgtaatgtaggctaaaaatacagattttgagatttatttaatcagaatccctggagtgttaatttttatatcaagatctcatagtgttttatttgaagtgacagggaggtctgtagatagctggacatgtatgggactggaagcttaggaatctttaagttcttccaggttattcttatgttcatttgtttattctgaaaatagcatctaatgtattttaagaaatggaataggcacatagtatacattgggtaacacaacagatagggtccccgtgcttaattcttagtcttgtgaaggtgacaaaaatacttaaaaatatgtgatcctaaattagaatgagtgttatgggagaaatgacagcaaatagtgatgagaattaatggggaggggaattgtctagatgagagggaaaaggtctccttgaaaaggggatgttaagtgggactgcaggatgagagggaaccgtctcttgtctatatgagaagtgagggttaaacgttttccaggtagagaaaaggaacaccatgtgctatgtcttagaaccagggatatccagtcttttggcttccctgggccacattggaagaagaataattgtcttgggctacacaccaaatacactaatgatagctgatgagctaaaacaaaaaaaaattgcaaaagaatctcataatgtttaagaaagtttacgaatttgtgttgggctacattcagagctgtcctaggccatgtggcccatgggctgcaggttggacaagcttgccttagaaggaaagagattggtcaggcacggtggctcacgcctgtaattccagcactttgggaggctgaggtgggcggatcatgaggtcaggagatcgagaccagcctggctaacacagtgaaaccccatctctactaaaaatacaaaaagttagccgggcgtggtggcaggcgcctgtagtcccagctacttgggaggctgaggcaggagaatggtgtgaacccgggaggcggagcttgcagtgagctgagatagcgccactgcacttcagcctgggcgacagagtgagactctatctcaaaaaaaaaaaaaagggaaagagattgtggagatccaggtgctgaagagaaggtctgcataaacagaacttagtaatgaggtggatggcctggtatgaggttgaggttaggtaagcagagccataacatgcaggactttctaggttcctataagatagttactactcatggagtttattcatgctttattccagctttggagccatagatacagaatactttggtcagtttggaaggctaggtgggatccaaattctaaacggttcctcagggttatactaaagtatttctattatcttaaaaggatgctgagacactttcgatggttgtttatcaatagcaaagcatcacagtggtgtgtttaaaatattaataatagcattgtatagattaacagtttgaatgaccaaaagctagaagaccagactactgagatgttacaggcttttaggaatgaaatagtttgcttttagaactcaatagcaaagggcagatgtctgagatgcctgaaagaatcatagaatgtaataatataggagctaagggagcaaccaaaaacggtttgtggaggggacaacattggtaccatgaagataaatggaaccctcagaaggcatccttaatttttgaacataataatttaagaagctgacttaaagtgacttaaaaggtcagtaggtagctggaaatgtatgatactagaatgcaagagaggcaggctagagatttggaagtttccctcttagtatataggggtaagggcagcagggaaggggaggtagaggtgccacagagtcatctgtatgggacttttttttttaccctagaactgctgaatcagaatgtgtgtgttttaaagtctctgtaggccattctgatggacatctggggttaaaatccattctcttagagttaatagttatgtaaagggagggaatgaagtcttaaagaggggaaagaaggtagtcatttcacaaatactgagcatcctgatcatcagtcttacgcagatcattctattagtagctggagctactatgaaaaaggaacccaacagaggtgatctttgtcttgtagggaaagtggagtaacttacactatgaaggagaagtgcagggtaccataagaattacagcagatagacctcatctgaggaaataaaacagacccgaaagatgaaggagacaaggaaaagtatctcttactgcattcagaagtgatttaagttgaagatggatgagcgaagttaatctactatgtgggcattgggcttccatttatactcctttgccagagtaaatgtcccccatttaagggtcctaaaggatggaagattgtaaaccttggaacacatgttttgtagtcagtgaattgtataaagtccctgacagtaagtgttttcatgccgtctttctggattgttcttaccccaggaatttacctagcttctttaggtctttagtcagatgtcaccttcacagtgaggtgacctaattatctatttaaaatcgcagccccactccattatttttctccatagccctttaatatcatctgacatactgtatggttttagtttattgtatatttttctgcctcttccaactagatcataaattctgagggtaggaacttctgaatatttttgttcactggtctatctgcagctcagaacaggacctggtactgaataaatatttttgaaatgattgaatggatgaaaagaaatgagtaataagaatattacctaagggggacagtggagataacaaaggctttttcggcttaggaaaggaacagtagctatttgagagtttgtcactagtgaggtgaactggcaaagtgaaggaaactgagcaacattctagaaaatgagaggaaatcaaatacttaggtgaaaggaagtaaactctggaaatacagaaggacacctcctaaggctagaacagatatttaggattgataggcacttctagctaatgactagggccttatatcctttttaattttctaggtggaatgcaagctttaggacttcacctgacagatccaagtcaacgtcttgttcagaactgtctttggactctcaggaatctttcagatgctgcaactaaacaggtaaattctgagtaaactggtgccatgggaatagagtcaagatgagtatgtgcttgtactgaccatctgtttttatctccataggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggtaaattgtcaaagtagaatttacctttgttgcagaattgaaaatgaagcatctctagctgttggatggctgtctaagcatagtgatcaataagtaggaattgtattccttagtaagtaggaagtatggctgcgataggggtaagattctgaaatgtttgtgtagtcagaactacttttagttgataccaatagatttagtgtggtgggaattttagggtaagaaaatgattttgttgagttgtatgccagttcttccttctgtttttcaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggtaggtaaattcttacagtgatacctggctatctaaaaggaatgcataaatccaaaggatcctgaacttctttctttggtcattggttccccccatccgtcttcctgaagagctaatgacaaagtaaataaataaataattacacatttctatggctgcagagaaaataaggcatagtgtggccccagtgatatttccttggacacgtccttcacatggtcagtcttacaaaggttgggttaggtgtttcataaagtgttctcatttaatttacacaaaggcccacttccttaggaagaggtagagtcataatttgagatcaaatctgtgtaatttcagagcctcttacccttgcctcatcatgcattttgactataaatatttagcagtccgttttattatcttttctgtgagttaaacttttttcatggacctaagaatattcagaaataagtagtagcatttctgtactcttaaccacaaaaatctcaacctgaagctttgatacaaagtttgtgtcttaaaagtagcttcattaaaagtatagtctaatgacatttctgatttctcagactttaagaccttattaggttagtttagaaaacaaagatggagcctaccagaacagatgttaggaatctcattttgctggttgctttgtgtatgtactcatattggggctttggctttcttcatttattactgttggtattggcccatctccatgaggtgacttaatagaacgttgagggcaccttttattttaaatctcttttctaggaagaagagagtttttgtgtccttgtaagaatcaagttatttataaaagctgctaaatgtagcagaataataaccccttttaaaactcaaatccagaaacaggagaaacagatggtacttacatattgcaaaagctatcttccttctatacatgaggctgtcagctgaatagtcttggaagagtgaggagtgaatttttctgctggcaactcggttagttttagcagttggtgctaaaacttggcaaagttttcaccaaatacatggaagatatacaaaaatagagggggcatgtaaaagaaaaacgttgacatagtctgagcattactttctcatcttctctttttatataccttttacccagaatgattggtgcccttactgtaggaaagttgtctttgggattcagcgctgtatggaagctctgttgcactgtgtatgggggaggggtgctgctttgaattagtgctgccaggaggcctcttttcagtgacattcaagttaatggaatccttcttccttcctgaactaattgcaagttacggggaacttcgggtatataatgtaaataattacagtctaataattgttcctcaaactttacagaggagaatgccctgtttgttaaccatgtttcttttggcaggagggggtccgcatggaagaaatagttgaaggttgtaccggagcccttcacatcctagctcgggatgttcacaaccgaattgttatcagaggactaaataccattccattgtttgtgcaggtatgttttaagtgaagtgttctaggttttatgtccataaaatttccagattgtaatgactaataacatttcagaaaattagggaccataatagggttaccaacatttaattttatgaaaattccctacattttttggtcagtaagagaaacattgagacttgagaagagggaggagatttcacatttcacttttatgggtgcctagaggggagagctgacctgggctgccagaggcagggcatagacccccaaccaattctgggttttccaaatcttagatcagttagagctgcctctgaagaaagggtttatagctaaaaaatattatggaaatccagtgctccagagcattaaacaccccaagacataaaattcagagaatattatttactacagtgtgaatgcctcttgcactctgaattgggaatgtttgcaccacagtggggggcttgccatgttttagctttagatttaattaggttttgtttgtgttttctccttagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtgggtaagtaaaaaggaaccaaagcctttagcagatgtgtacattgaagtctcagtttttcctcaagggcctttttctccttgtctcttagcgacatatgcagctgctgttttgttccgaatgtctgaggacaagccacaagattacaagaaacggctttcagttgagctgaccagctctctcttcagaacagagccaatggcttggaatgaggtagggaaatgtgagcagttatttatctggtagtttcctagagcaggtatggcagcttgttctttcctctcaaaacacttagtacacattcatttgcattgatgtttccctggcttgagtatttcttctttatgctgtctagcaactgctctgaggaagaactataatacaagctttaaagagtctgttcagaatcattacaaataagttgtgttatttaaaattataattcataagggagaaagatgaaaaatgttaccagattaaagaagatttttcaaaaggatgtaaggaaagaggcagtgttaaacactgttaagaggacagtttatcagtattttttactaaactttaataaaacttttctatttgaatttctgctatgaatttttcttcagcatttgtcctcagtacaggtggttccttgaaacattgtttctaataaaactagaacatcctgatattttatccattctatagagatcattgatggtacacagacatacagtggattatgtttgttgagtgaatggaaagagagattgttaggtttacaacgatgcagctcttgagaccggagtttaagatcagcctgggcaacatagtgaaaccccatctttagctgggcatggagatggatgcctatagtcctagctactggggagacgggggcaggaggattgcttgaacccaggagttaacagactgcactcagtgacagagccagactccaacacaaaaaaaaaaaaaaaaaaaaagcaaattaccagtgagtagtgtgttacttgggtttttaataggcatcttattaacatgttccaacttgagcccttaactttctccacctacccccttccacaaacctgttttcactgtcttctctgtcttagttaatgtcagctttgtctgtccagctgctcaggctaaaacttttctttcatataacacatcctatcagcagctcctgtttgtgggtaggcattttgcctttttttttttttttttttttaaactgctatatctctagcatgtagaacagtgcctggcagcacataataggtgcttaatataatatttgttgaaagaacaagtcagtgagtatttttaatgtgaggtgcaaagagaaaaaaaaatgtatctttgaggtgtggagttttgaagaacttccattttctaagcatttgtgtaatgttggagttacttgttccttttgtaatctgaaagtatgctttaaaaaaaattagtgtacttttgagaattttcattttgctttctattcttccttgctttgtgcatgtttatctagactgctgatcttggacttgatattggtgcccagggagaaccccttggatatcgccaggatggtatgtgtctcatatttctcgattaactccagatcaagctaaagttctaaaacttttatcagaagagccggtttgctcatctgggaaaccagtgttggcagaaaagtagtggcttcaattaaaagcagttcttaaattccagtcagcaacagtatctttaatggagcacagggaattcagagccacacaatgagtagcagtaggattacaccaccaacaaatacatgctactgctaggcctctgcagtgcaggatgttacaatttacctggctttttattctctttttggccagaggactcataatacctttgtctacaagctacccaaggaagataggaaaactcctgtttctaggctcagatctcgggtgggtttttacatagttgcattatcatcagggttttcttgaaaagctaatttaaatctgggtaatgaacatggaggatggcatagaccactaacaattataactgtcttacatttataaccgcatctgcttctacctaattatgaaaccactaaagcgcagattcttactgtgagaaataacatgtcaaccctaagataaaatatgttgaggtttcatggaaatagtgcctttccttagtacttttgtgggtgtcacttggcctttttgtcaagatagattacacctgccagacctcattattgtcttaatcctccttcccatgacttctcactgcctaggtggtcacacagtagattcctgcttcttctcctcgggaaccccaagtctcttgacaggggtaaatgcagagtgttcagggttagactaatgatgtgactaggccctgctggtgtgcctgtctgatggaaatagatgttatttgtgtagtctcatgggtggcctggcactgagtaattacttggctaaagaaagctggaggttgaagaggctagaaagcgttgttttctgacaagtttgctgctgaactttggatgccctaacctcagtgttaacgtctatgtctgcttctctcctctctcttttgccttccttcttgcctattttgttgacaccctgactcttctagatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 16 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 17 - protein, 232 aa, Mus musculus
MERCPSLGVTLYALVVVLGLRAAPAGGQHYLHIRPAPSDNLPLVDLIEHPDPIFDPKEKDLNETLLRSLLGGHYDPGFMATSPPEDRPGGGGGPAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 18 - protein, 232 aa, Rattus norvegicus; identical to SEQ ID NO: 17

SEQ ID NO: 19 - DNA, 699 bp, Homo sapiens
atggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctag

SEQ ID NO: 20 - DNA, 1892 bp, Homo sapiens
aaaccggtgccaacgtgcgcggacgccgccgccgccgccgccgctggagtccgccgggcagagccggccgcggagcccggagcaggcggagggaagtgcccctagaaccagctcagccagcggcgcttgcacagagcggccggacgaagagcagcgagaggaggaggggagagcggctcgtccacgcgccctgcgccgccgccggcccgggaaggcagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcgatccccaccgacgccccctgcaccgcctccaaccagttccaccaccctctagcgagggttttcaatgaacttttttttttttttttttttttttttctgggctacagagacctagctttctggttcctgtaatgcactgtttaactgtgtaggaatgtatatgtgtgtgtatatacggtcccagttttaatttacttattaaaaggtcagtattatacgttaaaagttaccggcttctactgtatttttaaaaaaaagtaagcaaaagaaaaaaaaaagaacagagaaaagagagacttattctggttgttgctaataatgttaacctgctatttatattccagtgcccttcgcatggcgaagcaggggggaaaagttatttttttcttgaagtacaaagagacgggggaacttttgtagaggactttttaaaagctattttccattcttcggaaagtgttttggttttccttggacctcgaagaagctatagagttcaatgttattttacagttattgtaaatatagagaacaaatggaatgactaatcattgtaaattaagagtatctgctatttattctttataatatcccgtgtagtaaatgagaaagaagtgcagagcaggatt

SEQ ID NO: 21 - protein, 422 aa, Homo sapiens
MPRSFLVKSKKAHSYHQPRSPGPDYSLRLENVPAPSRADSTSNAGGAKAEPRDRLSPESQLTEAPDRASASPDSCEGSVCERSSEFEDFWRPPSPSASPASEKSMCPSLDEAQPFPLPFKPYSWSGLAGSDLRHLVQSYRPCGALERGAGLGLFCEPAPEPGHPAALYGPKRAAGGAGAGAPGSCSAGAGATAGPGLGLYGDFGSAAAGLYERPTAAAGLLYPERGHGLHADKGAGVKVESELLCTRLLLGGGSYKCIKCSKVFSTPHGLEVHVRRSHSGTRPFACEMCGKTFGHAVSLEQHKAVHSQERSFDCKICGKSFKRSSTLSTHLLIHSDTRPYPCQYCGKRFHQKSDMKKHTFIHTGEKPHKCQVCGKAFSQSSNLITHSRKHTGFKPFGCDLCGKGFQRKVDLRRHRETQHGLK

SEQ ID NO: 22 - protein, 423 aa, Mus musculus
MPRSFLVKSKKAHSYHQPRSPGPDYSLRLETVPAPGRAEGGAVSAGESKMEPRERLSPDSQLTEAPDRASASPNSCEGSVCDPCSEFEDFWRPPSPSVSPASEKSLCRSLDEAQPYTLPFKPYAWSGLAGSDLRHLVQSYRQCSALERSAGLSLFCERGSEPGRPAARYGPEQAAGGAGAGQPGRCGVAGGATSAAGLGLYGDFAPAAAGLYERPSTAAGRLYQDHGHELHADKSVGVKVESELLCTRLLLGGGSYKCIKCSKVFSTPHGLEVHVRRSHSGTRPFACEMCGKTFGHAVSLEQHKAVHSQERSFDCKICGKSFKRSSTLSTHLLIHSDTRPYPCQYCGKRFHQKSDMKKHTFIHTGEKPHKCQVCGKAFSQSSNLITHSRKHTGFKPFGCDLCGKGFQRKVDLRRHRETQHGLK

SEQ ID NO: 23 - protein, 423 aa, Rattus norvegicus
MPRSFLVKSKKAHSYHQPRSPGPDYSLRLETVPVPGRADGGAVSAGESKMEPRERLSPESQLTEAPDRASASPNSCEGSVCDPSSEFEDYWRPPSPSVSPASEKSLCRSLDEAQPYTLPFKPYAWSGLAGSDLRHLVQSYRQCSALERSAGLSLFCERGAESGRPAARYGSEQAAGGAGAGQPGSCGAASGATSAGGLGLYGDFAPAAAGLFERPSTAAGRLYQDRGHELHADKSVGVKVESELLCTRLLLGGGSYKCIKCSKVFSTPHGLEVHVRRSHSGTRPFACEMCGKTFGHAVSLEQHKAVHSQERSFDCKICGKSFKRSSTLSTHLLIHSDTRPYPCQYCGKRFHQKSDMKKHTFIHTGEKPHKCQVCGKAFSQSSNLITHSRKHTGFKPFGCDLCGKGFQRKVDLRRHRETQHGLK

SEQ ID NO: 24 - DNA, 1269 bp, Homo sapiens
atgccgcgctcatttctcgtcaaaagcaagaaggctcacagctaccaccagccgcgctccccaggaccagactattccctccgtttagagaatgtaccggcgcctagccgagcagacagcacttcaaatgcaggcggggcgaaggcggagccccgggaccgtttgtcccccgaatcgcagctgaccgaagccccagacagagcctccgcatccccagacagctgcgaaggcagcgtctgcgaacggagctcggagtttgaggacttctggaggcccccgtcaccctccgcgtctccagcctcggagaagtcaatgtgcccatcgctggacgaagcccagcccttccccctgcctttcaaaccgtactcatggagcggcctggcgggttctgacctgcggcacctggtgcagagctaccgaccgtgtggggccctggagcgtggcgctggcctgggcctcttctgcgaacccgccccggagcctggccacccggccgcgctgtacggcccgaagcgggctgccggcggcgcgggggccggggcgccagggagctgcagcgcaggggccggtgccaccgctggccctggcctagggctctacggcgacttcgggtctgcggcagccgggctgtatgagaggcccacggcagcggcgggcttgctgtaccccgagcgtggccacgggctgcacgcagacaagggcgctggcgtcaaggtggagtcggagctgctgtgcacccgcctgctgctgggcggcggctcctacaagtgcatcaagtgcagcaaggtgttctccacgccgcacgggctcgaggtgcacgtgcgcaggtcccacagcggtaccagaccctttgcctgcgagatgtgcggcaagaccttcgggcacgcggtgagcctggagcagcacaaagccgtgcactcgcaggaacggagctttgactgtaagatctgtgggaagagcttcaagaggtcatccacactgtccacacacctgcttatccactcagacactcggccctacccctgtcagtactgtggcaagaggttccaccagaagtcagacatgaagaaacacactttcatccacactggtgagaagcctcacaagtgccaggtgtgcggcaaggcattcagccagagctccaacctcatcacccacagccgcaaacacacaggcttcaagcccttcggctgcgacctctgtgggaagggtttccagaggaaggtggacctccgaaggcaccgggagacgcagcatgggctcaaatga

SEQ ID NO: 25 - DNA, 12116 bp, Homo sapiens
gagggtgcgcccaccggtcccgccgggcgcccgcgggacgcgccgccagggccctctccgccgggggctcggcgctcgcccacctcttccaaatttaaccattacctaaatccgaagggaaatgagcaaacctctcggattgggtgtcaaggtattttcagcctcgttgggcgtatttatccccaagtgtttccacaacaagctatttcggggcctgcggggcaggtttcgctctgcggacgccgtggccactcgccgggctccaggccggcggcaccgcgggccggtgattcacggtcccgacccgggggtggtgcagccctaggaggcggcggggtcgggggtggggggggcgggtgaccgaggcctgaggggcggggagggtcctcggagcggggcgccccccacccctctctcgccagtcaatctgtgtcctcaatctgtggcttctctcgctgcggaagtctccctggagccaagaatagttcattttctttcaagtcatttctagtgcctaagtgtccggacctccaatttcccccatcccctgccgacccacagggagagaactgggaggactactaaggggcgcgcgggacgggctggaaaggccaggccccccaccgcctggccacttgcgcaaaggagcgcgcccggccgcccgacgggggttgggagcaggtctgggagggctatgcgagcgactcagtaacgctcaggaagtgaagcttgtggttttgggggctgagctcggaaggagaattttttttttttttaagtcagagagatagagcggtctctcccgaaagcaagattccgtttgaaaactctcctagcgcggtgcccgcgccgtgactccgcaggtaggtccgccgagcctgttctgcgcctcctgccctggtgggggcggccgcggggactcgcagagcactggcactgcgggggcgatcagagggcgcgggcggtttcccacctgctgcggaccgccgtgcgggagcccagagagctccggcagctgggttagggcgcgacccgcgcagtgtgactggaaatctggagctgggggcgcgcagcaggcggtctggtggttcggcaggggagccaaatccaccagggaaggaaacatctggtggggaggcggcggcagctgcgctcgggaggacgcccccttagcgccctcggctccctccttcctgggcccggacggtgaggagaggcctgagcgcgcggaggggccgccccacctcccgcgccagctgcagcgcggggttgccttcccacgcgctcggcccaggccccggggcccctatctcctccaaactctgtcgctccccacaggaaccagcaaagcgggccggggtgcgagagaggcgtgggcttacagcccgggtggggaggcggcctccgcgtccgcctggtctctggtggcgccagcccagacccagctccggcgctgacagttaccccgcccccatctgtcccgctcccagccaacgtgggtccaagctgcagcgggaccctcgggacaacgccactccgtttttcttttcttccgagtttcgtggctgtttaaagaattgggtttggggtttgtggcgtctaattgtacggacgagaagtgcaggaagcgacaaagctctagccctaggagccaccccggagggaggcggatggcactctcacccctaggggcattctggcgcttgggtagcgggaaacttcgcgggagccccgcgacacgtcccaggcgtcttttctcccaggtctattcccattcctccggagaaggggacacaatggggctggggatctggagcagggggcctgcaccctacagggaccaaggcctgtaggactcgtttgagctgagagcgccaacggacagacgtagactgtgtggcctgcatcttgcctaggaagccgaggggctcctagtccggcagtggaaacagcgcgaagccggaggactgcaggtcctgccccggcccagagttcccagcaccctcgtttctgaaccagccgaggccacggagaactgctgtactgcagctcacgtgtgaacccggtcaccatcgccttcaccccgggaggaaggcagattcgtttactccagaccacctcgactgtggggtaccgcccccggagccggctggagcctagcggcaggcgcagccacgccctcccgctgcgctcagatttcgacctggtattaggtgaactgattgggggttaatgagagcgacgccccgggcagctagttccctcccgggcccgggcccgacccccgctctctgctaatgcagcctgcgcgctctggcgtcctgtcttttttgtctgctaaattgtcggtgcactaccgactcgggacacctagcatttcccagtcaacgttcgtggatcgggctccacctccctaggacaagattttttggtgagcagaacggaaagtgcttttttcccgggacctgattcccgaggttaggtctccatggtctgggatggctcgccgcagcctcgaccggtgcccgccgcagccgggagtccaagggcaaagtttctcctacgtggggcactagtgaggggcgagtgggatcacccagatgcgagtttctcctggggcgggggttggtcgtctgttgttcccctcactttcctgtccctttgctctcccacctcctttctctggcctctgctgtccccaatccctctgctgctgtcctcccgccgccccacagttttatcttgtgttctgttcccccctccccccggtcctttcactccagttggtagctggctcttggaggtcttggctccttgccccttccgggtcctcgaccactgggcatccccggcccctaaaccgatccgcgtgtccccgccctccctcgccagccgtaaagcacagccaggcaggcgatgagtagctgagttggggtaacccacccgatgggaactacagctctccagggagtttgattgccggagcgagcttcgctaggaaaggggaggagctggggggcgtgggcagggaggaggaaaggggcctgagacagggccccgggacaggttttaccgctgagctgtgtcagtggcggcggcggcaacgacggcgggttcgcgccacctgtccaagtgccacctggtaagcgcggcgcagcagggtcaagcccctcctcccgtgggccctctgcgcgcctccctggcccgcgctctccctccgcctgggtgcccagtccgccgcaccggagagtactgacccacgtctccacctggttttctttccctctcaggtctcctccgggctggggctgagcaagccctcggagtgaccgtgggtgacagcggctccagggactcttggggcgcagtggggaaagtgccggaccaccatgccgcgctcatttctcgtcaaaagcaagaaggctcacagctaccaccagccgcgctccccaggaccagactattccctccgtttagagaatgtaccggcgcctagccgagcaggtgcgaggcgcgcgcgggccaggcggggctgctcccccggatgcctactgcacctcggcacaccattagtccggagctgggaggggctgccccaacgtcccttttgctgctgtttttgtttcctactgtcctggttcctccgggtttgtctcctaggtgccatggcctctctgcgcctgccctcggatccgagagggttcccggccggggtctgggtggagaggggaagacgctcggctgccctggtcgggggattgggggagccttcagcaccctcagactcaaccggtcccagcctgagcccctcacctgcctcctctttgcagacagcacttcaaatgcaggcggggcgaaggcggagccccgggaccgtttgtcccccgaatcgcagctgaccgaagccccagacagagcctccgcatccccagacagctgcgaaggcagcgtctgcgaacggagctcggagtttgaggacttctggaggcccccgtcaccctccgcgtctccaggtaggaacccactgggaacctcttgggcgggagctgcagggacccggcagtgctgggggggaattggcgcgaccttgggcgtagaaatgctaacggggagttggagagtctttccgggagaagggagctgattcgtaggggaaggaggcatccggcttctctgggacttggacagcttgcccgctggggctgctgcctccatcccaggcggcaggaccctagctgcttgtcgcttagattcgtttgcgcggagctggccagtgacggaaaacaaaccagtcgtttcgactggcggcaacgctgacctttcattttctgaccagacctgactgttttataaagtgctaggatcctgcaatctagacccccaaacctcaaacagagaacagggcagaacgggccaggcagaggagctaggcgctgggcggcagggagggggcaggacgaaaatctcagcccgcggcttggtcttcacaggcgcagattgggggcctgtttcatttttcgttttgccgggttaacctagcctcaggggcctgctctctgggtttcatttccagcgagcaatccagcttcaggcaaactaagtgaccacacgttgggtgggggcgtctcgagtcccggccgggggaaggaatgagcagaccagccggattctgtcaagggccggttatatccagaatatgtttgctagttttagaagataccaccacccgtcccacaatcagtgagttgacttggcgaaaaccatagctccagcaagtgtgtctgggagccggcggcgggaggattcttcctgccagggcgtcaagtggccagacaaggattgggcgcgccccgaacccctccgaacgaaactccgggtacagcctctcactgaagtggccagcctgaactggagtgttgtgcgcacacacacacacacacatttgtaaatgccgtatgcactcacatgcgttggggtcactagttttagcaaaattcacgtgggtgggggcgtagcaggccgagaattcagagctgtctccttgcaggtggtggctaaaccttatgagttatagttattctctgagaaattcaggttccccgcctccatcaaactgtaacaggaatggggagtatttggctgtcaatttaagcccaaaagcccctttcctgctgctcctttgctacgtacactgggcacttaacttcgtgaaatcttaataccttcgggtttattcagacagcagcctttcgggtagttcggggccgcatttatggaccttctccctccttcctcttggattctgggaagaaaaagaattgaatgggaacatgtaggggctgggagagtgcctgcgctggtggctggacccttccgcccttgagtgctgtgaggggccgaacggccgccaccttctccttcttaacagctcaactacgggcatttatagatgcgcccttccctgtaggatctccaggtgcgcgggtccagccagaaaagatcctcggaacgccgagcgcctccgctgcactcgcacagaatttacgacctcctctcccgaggtcttttcaatgatctgtttactgttctgcctcctatagtggcctgcgaggccccagggcccgggccacgttttaccctggggcgagcctggcacctggcgcacgcagtgttctacaagcgctgggtgccccgcagtccgcgaacacgccacgctcgcagccgcagcccggcggcctccgctctgccgtctgaagcctgaccggacgctccccttgtgcctccacagcctcggagaagtcaatgtgcccatcgctggacgaagcccagcccttccccctgcctttcaaaccgtactcatggagcggcctggcgggttctgacctgcggcacctggtgcagagctaccgaccgtgtggggccctggagcgtggcgctggcctgggcctcttctgcgaacccgccccggagcctggccacccggccgcgctgtacggcccgaagcgggctgccggcggcgcgggggccggggcgccagggagctgcagcgcaggggccggtgccaccgctggccctggcctagggctctacggcgacttcgggtctgcggcagccgggctgtatgagaggcccacggcagcggcgggcttgctgtaccccgagcgtggccacgggctgcacgcagacaagggcgctggcgtcaaggtggagtcggagctgctgtgcacccgcctgctgctgggcggcggctcctacaagtgcatcaagtgcagcaaggtgaggctcccgagctcaccacctcgcctgccgtgcgcccgcttcccctacccgcgcctcgcctgcgccccgcggcccctctcagcggccttctctctggccccacccgccttaggtgttctccacgccgcacgggctcgaggtgcacgtgcgcaggtcccacagcggtaccagaccctttgcctgcgagatgtgcggcaagaccttcgggcacgcggtgagcctggagcagcacaaagccgtgcactcgcaggtaagcgcggggcgcaccgccgcgcgcggccctgctcggggatcttctgcatctcctcggtgcagcaccagccactctctgcctggaagttttctcctcgacttcccccagtttcctcccccaagccctccgctgcgtccccttgccctggtgcaggtgtgtagggaaaggaggattgtggccggctcaggccttgaggcagccctggattttggtgtcacaccactgtgagcctcgagagtgtgatcctcattgttactttgggcttgaggtaggtttgtatgcactgattcgtgctgctgatatatcagacttactagctctgtttctttgtgcctattcttttcaccaaatggttgtcacttaatttgcattgacccctctcgactgaaaaggcaggaatctcagctcatttagagcatctagtagcatattcaccccgctattcattctttccttccttcctttcttttcttttcttttttcttttcttttctttcagagtctggctctgttgcccaggctggagggcagttgcacaatctcagctcactgcaacctccccctgtcaggttaaagtgattctcgtgcctcagcctcctaagtagctgggattacaggcgcatgccactacagcacagctaatttttgtgttcttagtagagacgggatttcgccacgttagccaggctggtcacgaactcctggcctcaagtgatccaccagcctgggcctcccaaagtgctgggattacaggcgtgagccaccatgccccaccgccgctatttattcattcattcattaataaatatttgttggctaacttccaggtgccaagtacttaagaatcttataacacatcaggtccttgacagcatgcccacatgaagattatagtttagctgagagatggagagtagatgagcaagtaaatatgccaatagctatctcaggagaatgcctacttacgaaggctaaaaagagtattagcccatctcccccagcacccacactggctggggggaggtggcatctcaagtgactgaggtctaagcctcctgttgaggagggtggagaagtgtgtgctaatgggtgtcaaaaaaagcagggtgtggatatgtatttgccatggggtgtggaaggttgtgggtgaagaatgttttggtagaaaaagtgttgaagggccaggcacggtggctcacgcctgtaatcccaatactttgagaggccgaggtgggcgaatcacttgaggccaggtgtttgagaccagcctgaccaacatggtgaaaccccatctctactaaaaatacaaaaattagtcaggtgtgatggcgtgtgcctgtagtccctgctacttgggaggctgagacacgagaattgcttgaacctgggaggtggagggtgcagtgaactgagatcgttccactgcactcaagcctgggcgacagaggagactgtctcaaaaaaagaaagaaaaagtgttcaagggattttagggtcagctgaggggtgaggagagcagcagtctagttgactgcagtaggagttctgcatctctctctctctctctctctctctctctctctctctgcaggaacggagctttgactgtaagatctgtgggaagagcttcaagaggtcatccacactgtccacacacctgcttatccactcagacactcggccctacccctgtcagtactgtggcaagaggttccaccagaagtcagacatgaagaaacacactttcatccacactggtgagctaaaaaggcccttggcttgtaggaaacaccctgaggccaacattactcatcttctctgatttctggccccagtgagtggtggatgaggcctttctgatggagttattctctgctctgtgttaaagaaaacaaaggggtgggttctttggttcatttaccggcataattctccccagagccaccttgatttggggttgtgtctgaaaggccactcagcaggtcagctcacaggtactctatacttggaaagaacattttcctttaggttagcagctgcttcccctgctgcctgctctgggtgaaatatgaagctccagggtcctcttagagagttgctctaaagcttacctagagattgaggactttccctaaccacctggccttttgtgggagggactcgtgtggactctccggctgcattttcaggagtctgagagcttattctgattgaagaggaacaaataatggcaaatatgattaaactctctgctaagcattttttaaatgcattatttcattttatgctcacaacaactctgagaggtagcgactactccttctccccattttagagatgaaaaaaatgaggctaggtaatctgcccagggtcacactgctagcaaatgacagggccagagctcaaattcaggtctgacctctcaaatgttcactcttgaccactgtttattgtattttatgttcagagtcatgaggttggtagacagaaagcttctgttcacttattgcccttttcaaaatatctgcaagttaatgccataataatgataattccttacctattataatgctttataatttacaaagtactttcaaatctatcatttcatttgattcttattgccactcaagaaagtagaaggagctgctcttaccatcctgaaactcagaaagagtgaatgatttatcagaagtaagactgaatgaataatgtagccatgtaatgctctggcttttaatcctggactgtttgtctaacactttatgtgcgggtgggagttttaatgccaagaacactctaatagtcaaaagacatttacatgagacccagaatttctgaaaattttattgcagaatatgaatactgatttagaacaaatcacagtgtattctaaacacccacccttgatgtttataaatatacttgggtaatgtatatatttccattgaaaacccagaaaagtattctactttaatcattccctcttacctgaaatttccatgtaattcactccttataagtaaggtattcaggacacttatcaaaatgcaactaggatcttgactgaataaaacattaagcccttatcaaacatttacgttatacctagaatttgttttctcagatttgtttgaccctaaagggatagaatacattttgatgggtggtttcttatcaaggaaatctgaagcatgaaaacagaaaagagtttttagcaaggaggacagagggttcctcaaaaacaaacttcatctattttatactttttccaaggctgagccctgactataatgccatgctgggctattggaaattcatgccatttacccaacaacacatgagatggggaacaagacaaaaccttcttgtgttctcttatttattaatttgtggtgaagaattgctggtatataaagaatcatgtgattaaccccataaaattaaggaaaaatcaagacagtaaagtatcagctgccttaatcctttgtggcccaaatgtggatttttaaaataagatattgaaaaacgtatcctgcacatgtaccccggaacttagaaagaaagagagagagagagagaaataaagaaagaaagaaagtccatgttaagatgttttttcagatataatctgctgtccttcaagaacaagaaagaagacgggctcactgatccatacaaactaacacccacttggaaattcagatttgaaaacttcctctgaattagaacggagtcacacggttttaggacagcttccccctccccttcctgttgaacatctgctctgagtgttcatggcttataaagtcaggggagtcctcccggggtagattcagctggggagggcacgtggcctttgctctgtttccgtttagcaggaaaccgtttgaggcctttggctgggaaccccccttcagaaagtctccctttcacctggtgcccccatggtgcttccagggactcgcattgcaggctgggagtcagttcaggttgcaacacgtcaccctccaagttgcttgaaggccttagactgtggtgcaaccagctgctgccaagagcatgtgggtcacagtgggtcccctctagctttatcatagactcatactttctcccctccccctcccatccccacaggtgagaagcctcacaagtgccaggtgtgcggcaaggcattcagccagagctccaacctcatcacccacagccgcaaacacacaggcttcaagcccttcggctgcgacctctgtgggaagggtttccagaggaaggtggacctccgaaggcaccgggagacgcagcatgggctcaaatgagcaccctggctggctgcaagcagcagctacacaacactacagagggcagcctccctgcttgccaccactctgctccctgcttgcctccactcccttctgactttccagaccccaggtccagtctgcagatcctaccaggttgctcctccttcgccttacctcctggagctgccagaagaaatgaggtaccttttcaaagtgcagccgagagtgagaaccaagtgactctctaggcttcggacacaaataggctcctctacacctgaagacaaaggcaaagtcaaatggggaccagaataaatcttagaccccacagtccttcccatttccagccctaatctacagacaggaatgcccttcaggtttcttccctcccccctcttgacctaccccagatatttgtgtggaagaggaggaatcaccatttacaaggtggacaaatgctaatatttttatctagaaagaagagtgagtgttaacttttatttttttccttctggggggtctgttgactcctttcttttgggtgctgcctataaatcttggaggaatcatttctcctcctcaaaaactgattcagaaactgacttggggaaggaatttaatactttgaagtcatgagatgcaccatcgaggctacccccaagaagaagcagaagagaagttggtaatgagaggggattagaggtcctcccttcagtagggctgtgaaaacctcatcactggaggtaaaagcacaagcaatgcctgtggacaagatgtcattcattcactcagcaaatgttcatggatcaccggctaccaaggtaccaggcaccatgctaggtattggggaagagagactgaagtcacaacccctgactgctcctcaaaagctaacggttgcacctccaagtggctgggtctgttcttactcttggagggaattctgagaagacagcacagaattgtaaaccttcccttttgacccttttggattttatcaggtgtaaacaaaaagctgaacagttacttcaaagatatgtgtgtatattcagttttttattgttaagctgatattttaaagatttctgagctagcaggcatgtgggaaggaaggctctgtcttcaactctttgaccctccatgtgtaccatagaggggggaaaggtggtattttcactttgatgaggttggtaaatgtttttagatcttctggtaagcattatgtttgttaatacatatttattagagtgatgttttaagttaataaagtattaagagtatta

SEQ ID NO: 26 - protein, 270 aa, Homo sapiens
MVTFATILQVNKVMSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 27 - protein, 258 aa, Mus musculus
MSILFYVIFLAYLRGIQGNSMDQRSLPEDSLNSLIIKLIQADILKNKLSKQMVDVKENYQSTLPKAEAPREPEQGEATRSEFQPMIATDTELLRQQRRYNSPRVLLSDSTPLEPPPLYLMEDYVGNPVVANRTSPRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 28 - protein, 258 aa, Rattus norvegicus
MSILFYVIFLAYLRGIQGNNMDQRSLPEDSLNSLIIKLIQADILKNKLSKQMVDVKENYQSTLPKAEAPREPEQGEATRSEFQPMIATDTELLRQQRRYNSPRVLLSDSTPLEPPPLYLMEDYVGNPVVTNRTSPRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 29 - DNA, 813 bp, Homo sapiens
atggttacttttgccacgatcttacaggtgaacaaggtgatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatga

SEQ ID NO: 30 - DNA, 63186 bp, Homo sapiens
agttgaagctcctctcccttccgaacagctccgcgcaccgccccgcgacgcagcccggcgcaactactttcttctctctcctttctttcttcctctcctttttcccctgctgggtagtggctgcggcggggtgggggagactttgaatgaccgagctcgcgtccacctttctcttcatgtcgacgtccctggaaacggccacacggatgccatggttacttttgccacggtaaggggaggcggcgggcaccttgggtgggcaggtttggggatgggggtccacgtggggagggattttccagtggactggtgcggggggccccagatccgcatcccgccccacccccatcgcgccgcgctcactcactttcccgggcttgtgtcttccccaaagtttgcgctgggatctgctcaggccgaagcgcaaccgcagccaccccgctacacacacacacacacacacacacacacacacacacacacacacacacacagacacggacacccttctccacctcctcccctcttgtccctcggctgcccaagaagcttccctcaatctgggaaagtgatcaggtttaagggacctggattggaaagggtgggggcagaagaggggaaatggggacgacgaaagagcaggaaagagattcaacagaatcaaccacccaccactcccaaccgacccgcctgctcctccgagaaagctcctagcgcatcctataacaaaagggggtggcagacagaactccgggcggggaggtgccgcggcagctcccctgcacacgccctgcactctgccggccgctgagcctgattctcagctcgccccagcaccactctggcccgggcgtgggctggggggaggggacgcgcagctcaggacccggaacctcgcgttccagttttgggagttgggactcactgccacgcgccgcgtacctgcgttggagttccccgaaagggttttttcagaaaagacctcgcgccccgggctcctcttggccagcgcccacccggtggccaccccaccctgggcctttgcgcagatgttggagctccgtacgcagcccgcacatctgggacccctccggggagcggcgggcacccgggcccggccatcccaggggatctccttgcggtatcgtccagcctgttctcggactttgagcggtggcgtgggaggccgggagacctgggcacccgcgcagccagccaggtcggagtttaaaggtcccacgacggaccgaactgtcccattgccccagagctttactcagtggtggatgctcctgatgaaatttgggacgcttgggagttgaaggttagggacaggaggggcgagggccgagggcatgggatgggggagtaggattctgcttgttgctctccgcgggagtgggtgcgcgtccaggaggcgctgcttctttgcgggagtttggctgctgcgttcattcgtcgtctgcgcttcagatgcacggcactgagacccttgcgtccgacggtgtcggggctgtggactagaaaggatcccttttgctggaatcgaggctggggtgggattgccggtgggggaaacaccgaaaagatcgtctggcctcggcctctggcggcgggcggcaggttctgagtccgaatggaggttgctcccgggagcgccgggctcagagctagagagctcgggagactgtgcgcctgtggacttgtttatgtgtgtgaagaggcgggggcgagggcctgctgagaggggaggggagcctggaaggggtgggtgtttctcctggagcctgatgtttgtaactcagctgattatggagtgcactgagcgacctgctttttaaataaagaggtgcccgctcctaccccgcaaaacagcgaacgaggagaacatggaagcgctctgtcctaaacgtcaggatgggagaaagttgtaacatagaggagacttttccagaggtcctgttttcacaacactcagaagttctccagcgtactcagcctgcctcccgcctgccctcaattcctttttgacatgtcacacaaagaaagctgaaaggtagaatgtggaggataaactccaaccccctctgccttgggcgcaaacacacagacttaggtatcgtgttaggaggtaagggggttggaaaataatgcaggctccggtagacagtgttgaagggagatagaaagtctggggtatttcccctagggagagtgtgtgggattttgtgttggtgaggactggagtgtagctggacttagagggtttggttgtgtgtgaatgggatatgcttatctatggagtgagattgtgaccattgagtgagtggtcagggagggatggggatgtttttccaaagtgtgtgtgtgtgtgtgtgtgtgtgtgtgtgtgtaggggaaggtggcaaaagagctggagtcctatccctctttggtgcctgggaggtgtgtgtttctggggagtctgtcggatgggcatctgtgtgcttagcgggagacaagagcttgcaggaacataggtgtagctaatgcgaaatctgtgcctatgaagggtgtcagagcaggaggagaggtgcagcctgttgaagacagatggcttgtgcatgcctgcacaaacacatggagtgggtgtggtgtaggagggcggctgtgtgtgtgagggaaaggtggatgcatgtggaaggttgggcgcctgaggattaccaaagggatgtgtgtggggggtgggggaagattatctgaggagcacggggtttgaacttcatgagggtgcactgggaaatttggctggaatggcactggggcatgtagggggtagaaaagtgtgagctcagatcctctttatggaaagacaaagcatttgcagggaaggacaccgggttggtgttggtgtttgcctcccccctccttcctccccagagagaagagaattggaggaggcttagaatgagggatgtacatttaaggggaaaagtccttgtgcattggcactgggtgggaacaggaagagatgtgtgcataagactgaagtaagtagaggaatgcactatctgtgcctggtgtcagggatgcatgttgtataatgttttgtaatgggtttcttatgtaataggagaattcactatcaccagttcatattgataatcaattaaagctaatggagtgcctactggatgctcagcccattgctgggtactggggcatggcacactgccctggaaaaattccttctccaggctcagtaaaactgatgcaattttgccagaaatagacagagctcgacgcttgttgaggtttcttcaaggtctataattcagtttctagagatgtttatggatcttgggggccaaagggaagctgacttgaaatatatctctggttttgtaaaaagtggggcataaagagagggatgatgtagacttttgtgtattgggaagggagctggacggtgttgttgtttcttgtgtggggtgtgggctgggaattggagggtgctgcacttcaggtcagaatgcaggtccagatccctctaaggattgagtttctaagagtacacagagcctccttcatagctggaatgagacctgttatgtggagctggagaatggtgaagtcatgatcaaggaagagatggactgggagtgtttgttcatctttgatgttttctgggctacgagtgtaaagctaacctgagtgtgtgttgggggggtgggtatgcatgcaggtgcgtgcacatgcactttggcctggagtaacgtaactgggagaaaactagcgaggtggataggaggggtggatattctggctcttgtgaagggggtgccagtatgtgtttttgcaggaattctgtgtgtctggggaaggataaaaattgtcttggatactcagatgtgcatctttagcgaaaaaaggtgtacacaaaagaagggccgtagactttggggaatgagacaaaggtgtcgttgatcgggtccataccctgagatggctggtgaggcaactgggtaggaacagagtgaaggctgtgcttttgtgatgcacacatctgcctgtctggagcagacgatggggatccccatttgattaggttctgaccaccgttgggcatatgtttgggaggactttatttgactcatgacatttttgaattcatgaagttacagctgtgccattttaagggtctaccttatttttgagagggaaactcatctgtgtaagagatagtttatatagggcttgactattggtcagaagatccggggcagatatttctgagagcagggtggtgaagtgcaagactggattgggaatgtcttagttctagtccagttctaccacttgggaaaagtgtgactttggggacatttttaatctctataactcttctgtttctctttttgtaaaggggcaatataatgactcaaggagaaggaaagttcactcctcttttcccccatcaaaacttgatgtgtttagttgtatacagccatacaaagatgaaatgtaattaaatacaggtgctagcacgtatgtaggtcattgtgaaaataggatctggacccctttctctttacaatgcaacttaatcagtaatcatgatgcccacagcatctctactgctcactcattgtgtaaataaaactttttataactgcctcagtttactcatccagaagctggaggcagccaaacctagattatagatgatggaaatctttaagatctttgagctattcagaggaaaggtagtctaagaatcaatgttattaatgatcagagacagacatttctttattttttaacatcagctatgcagaccacagcaccaaaagaaatgtgacgtggagaacagcaaaaataataattgcccttgacataggcaccatttgaataaaagaagagcaaagtctagcctgcctggtaagaggaaagcctgggctttgggtctttgaaattctttgtgcccagtgcagcaagttgggtacagccactttccccagagatgaagatggagtcgtagaggggcatggaaacacactgaaacctccgattcctcccagaagtcagtacatgctgtcaactcctggctcagagtcaggctcccatccttgagttcagtggccataaatagtcaggagctaagctatttccaagaggcactaaactaattctgttgcttttcttggcgagattgggccaattaagttgattggaagaggtcactgctgatgggtctcacattccccaggcgggacaggccagcagagtttaattacataacatttcccatgtcttcacttccactcccagatgtatcagatgttcaggcctctcctctttccctacattcaggatgcctgacggagcaggcggcttctgctacaagcttctccagaccttttcctgagcagttacagcactcccaccccaactggagggaatgcaagtctgtgcattgcctggaagtgagagaggggatgcaggctgacacagaagatctttagagcccaccaccctgcctcaagcctcacccgcggctgttttactctttaacgaggcaggcagtttctggcgttggggatgttgaactaatgaccgttgctagggttttagatttagttttatttaaaaatgagtttgttggaaaaggaccaaatcttctactggagacaagcctaagccccgtgtggtgctgctgataaggtctgtggatgtactttgtgcatggacaggcatgtgtggagtaagaggagagcacaggatgtgcgtgcgtgcgtgctgggggacacgtgagcgtaggagatgccctgggtagatggggcatttgtagagcttatgttatgggtggcaggtatctggatgcttggctcagattggattgtcattggattgcttcttacaggtgggacagcctgagaaaaaagttggggacaccttccttcagatcatttttatatgggcatgttgaaccttgaagtatgaggccagtgtagatattacctcattcttgcctggatggatttctaacaagattgaggctggacaaaaaaaaaaaaagtctttcagaggactcattttagaacctgtgagaacacatgtagatggccccagagcgttaaagtattttttgttgttgttaatagttttccataactcagtcttttgttttacctaaaaatactgagaggctagaggttgcacacacccccacatggtcagaatgcatttgctgttcatgtccccatctgtgccatggtccttctcaaataggattaattgggaactacggagagcttgccgcagctttggttgtcctcacaggacaaacctatgactgaccgtgatgtcattgaaacagaacagattttgacgtgaaatttcgtgtggtctgttgtgattgctcactttttagtaacatctccagagaagacatttgttgagccccatttttttttgtgggtttgttcagtcagagatttgttggaagatttaaggttctccaacattcaaggccagagattttggaggaaacagtagctaacaagagcccaggccagggtgaccttcagtagcctgaggcagcaggtagctgcctgacttagaagtgaaggaccaggttcagtgactgggctctgtcactaattagctgctactgtgtgaccctggacattctctgtaggacatgactttgccatctgtaaatgaagggcttgaattagaagatctcccagtcccattccagccctgtcactgtaggattccacagtagccacatcctcctacctaatcagtgctaattacctcacttggagtgcaccctggcacgcccttaacgagctcctgtttttatctttggggaatggctgagtcaaaggcagaaaagcgtgcaaaaaatttgccagagtgtttccttttggccaagaatgctctaaacattggcttagcctacacagggtgtgcagagctgaaaggggtgggatttggctttctggtaggtctgatgctaatcattcagcttccacgtgaatgtctttcagtggcaatgtctctgactaaggtaagcatccatctttaagatgatggatgatgatgatgatgacagttaatattagagtacttactgggtgccaggcatttttacagctattacatggcattatctcagtactcaaaataatctcatttacagatgggggtactgaggttatgtgaggttaaggggtatgctgatgttcttggctttccagcctgaagtctgtggcatcccaagcccatcacctgctagtcagattctgttttttatgtctgtgatgtcatccagatgggagctgggtccatcttcttctgcaaagttggtcaccccatagggtaacctgctccctgcccccggaagcctccttgtcatgtgcaagacaaatgtttaggttttctagccacctgctggcaagcatgtgtcagatctgtctcagcctccattggccctctccctttacttcttgttctgttgacaagggaacctgcccttacagaatacggatattgatttaagccacaattgcctccttgtaaactctataaaattggagctggacatctggttttgatccacacgcccacgagaggtggtaggtcccagcattcttgagtcctggaattgcacagaatcaaagtgaaaaagaatatgaatggagagcatgaggtatctttagaatttggaagatggctttctttgataggctagggaactcacaccatttagactggggccaaagttaaggtaggtttcttgctgggaggttggggggcaggggtcactgctaaagactgaggtccccaggctgtgtttgatgcagagcagagcagctgtagagccctccagctgaggatttgccactgcacaccattctctgggcagtaccagaaatagagaagccaaacaggttcttggggtggatgttgccctgtttgaagattgtgactaaaggctcattccttcttcctccactgatttcctcaccatcactaccacatatacatcatcaagacagttaaggacaggtgactttgttctgttgcttaaacatatttgcatgtacaaataaatgttgaacactaacctaatgcctattagaccataggtactcaaaaattatgtatttactttgcttaaaataacctttcatgtgtagggtgataatttttttcttcatatggagtatatagccttttaaaaactgatagccaggttcttgcaatttacttcccccttccctctgtttctagatctaagaccccagcatttaaaaatatccttctctggctctaagtgatccctcagggagtttgtttcaagttctcaatctgagattagtttcatcccacatctagaggacctgcttctaatggttcataaggtacttccactgggttcaagaggaagacacagctctagaaatcacctgctttcccaacaggtgtgtcccatggtggggaagaagcccagtcctcctaacacccacctggctaccttaaatagcttgccacagtctccctgcagagagcccagactagctgtgtgactttaagcaaatcactttccctccgtgggcctcttttcctggaaaagggaggagttggactcactagagggtcacaaggaccctaccagtgtgacagatcacatcttccatgactccgctgctccctcggccccacactgtggcccatgcctgggattttcaggtaaccctgctggcctagaggacttacatagagtgatttggagggggtggaggaggaaattaagttatgtgatgccagataagtcattgaacatacggatgagtgcatgtttatgaagcactgaagaaacaagcttgccagattcgtggcggatcttggattggaagttgggagtatgcgagaggtttatgtgggattaaagttgtctgtgggtagctgaattcaataagctaaatcagagagatcccaaactctgcagagagatcagaccccgcctctaactccaacagaggctaattacaattttgggaagtcctcgaaccacactaggtctcacttcctcatctattaagagctactgactttgattatctctaaggtctttcttgacttatcattaattaaattgtgagaattgggagctagtagaaaaatctggacaaatgcgtggatggagaaggggatacctcaaatggcctaaatatagagggaaatgttctgtacaagtgatacgtttattcttgaatcggagatagcattggaggatatattctgtacaaatattgtgcttcaaatgcttacccctccatcatcatcaatgatattaaaatcattgcttaacctgggatatttgcttaacctgggaagcctccctgcatttccccagagagcctaaggctcccttcccagcctcctgatgccacttagcagcctgtgtttaatttgcattcctagaatgtgacttcacagagaaagggatctgttgttttcatttttgtgtcccccaccttgaatagcgacagccacaggaatgtgttgagtgaatgaatgcgtgaccagccattgtgagctcagaatgtgcagcagcgatttgacagtgccaggaacttggctcagagaggcagtgcaccttgccagggcttcaaaacagtgttacctttatcccccttttggcctgtcccaaggctaaggtcatgaatgctcagcgcttggtggccagaaaaggctgttcttactcccttttagagacctttcttccccacatcttagcctgcttagttctcctgcaaattggggctcttggtcacagggaatcgggtgtcattgcatacgcattaggataactcctcgttagacactcggaaagcaactcaaaacacacgtgtacagattattcattaagcagtctgttggtggtcacgtgctaaatgttggggaagatgcagagatgaatgtagtgctgctggccttggggtctggaaactggtgaaggaggcagtcaggttacaatcatagtgctccagggagaacaagggaagcaaggaagatggaatacagggttcccacttcaggtgttgttttttagtacaaaccatgattgagaatcctcttggttcaggagcaagctggagggtccaggaatggtctccgcttccacatgacagtggttttcttgtccctgaattcccagggctagggtgatggatgctcagagaccattgtgatgggagcccttggtccttgtggggagagtcgggggaacacgcatgttgtacaggtgttggggcttggggaggtgtaggtgtgcatgctcaccagggtacttctcagaagtatccctgctcgtggcatggtgggggtgagtccacctcaggcccctaagctgtgcttctacttcctttctaattatacagcatgtattgtctctgtgaccacttctgacacaggaacagactgccatctactgtccacttctgtcctgagtccctcctgctcaaggaggcatttattttttctcatcctgtttttaatagatccggacaccaggagaaacagacgtggagtcctatttagtaatttttttagaatttagaagggagccttcaaaaaacatagcacatctgtcagatggtttccgtatctgtttacaaagtattattgtaaggggccactctgctgccactgagtcttgctagccgctgtgcgaggtgccgtgtgggataccaagagatacaaagcaaggccttaccttcaacagccttgcagtctacttgcgaatacagtgtagatttgcagagaagcagactacaggatgtggcagtagatatttggtaagaggcatctgggtggtagggactacagaggttttgggagtttggaggaaggaggagcccttataggctgggggatttgggaaagaccaatatgggtctccttttagcaatcccagtgtttaggaagaggcttctaagagctgcgcgatgttgtgggacaggctatgaactggaaactcaggaacctggagggcctgaggtctagtccagcttctggcagtcctgccctgtgacccagggccttgggttccgcatcctcaaagcaaggctgttggtcttcttgtcatgtaagggcccccccatccccgaccctgcatcatgtaccactcttggatgcagtgggaggtggtttcctggtttccagcttgtcaagagcaatagagtcaattggatcccatgcaggaaggatacctggatgcagggccctgtgctgatgaccccacatgcagcaggagagagagaatttccccaggggagttttgggtgctgttaaaatacaaaggggggaaatgttccatgcaacctctgcatgccactcctctccagagctttcctggactttccttccaaaaatatatgtgcatggtgctttgcgtccaagatagctaccccaaaagaaattgtattagtattctagcattgttcatagctcaaatctaagattccttcacattagaattcattccatgattgtttttatggaggttttctttgagtatttgagacttttggtagctgtggattaaaatgaggaaaagattgttttctatagctcttttactccttgtctctttttctctgtctttcttagcatttaaaccaccaccaaggaaacctccagggtgtttattgcatattcttgccttttgatgtgtgtgtgtgcatgagagagagagaaaaaaaaagaaagggagagaggagaggcgagagagagtacaagagagaaagatattttagactgtggtctattagcatgtctaggaaatcaagttgaatttggattgggttacaggttgagagacctcagctcgtcactgtaatcctctgacatttcccttttactttttggctcttgtctgaatcaagaatacatcttcccgttccctcttccatgtttacatcttcttttggggcagctgtataaagttactgtctgtctacagcaagtctccttaattcttttggggactgctcgacagaggcacagtcaaggatagaaccattagagacggtcagcttttggacaaagacaaaaaaatatgttgaggaataggagaaatgttccaattgtcgattatgtgttttcctgtggtatgctgatgtctgtgactgttgtccacagaaagagagcgtgagcaggggctggggcagagataaaagggagattctccccaaaccaacaaatgtggagagagagcaggagtccccagcaacagatatggagccaaacaaccctgggaagtaactaggaaacttcctggtgacctgagatgtgttttgtaatcactggtgaaagtaggaggagatggggtagaggagagttacaggtagagaagttccgtgggaggagaccccaaggtgagagaaagcagggacttaaagaactgaaagaagcccagggaggggatgaacagagaacatcccatatatcccatatccagtatggtggagaggtgggcgggagccaactgggagcttactttttttttcccaggggcatgggaactatggaaaactctgagggcacaattaagcttttgtttttaaaaagatccctccggctgcagtgtggctctgaataaagagttgttcaatgaagaaaggaatggctgatggttggattgatggaagaattaatgaatgagcaaacgaatgcatgagatgcagagggaacgcagcaagcctgatttgatctctggttactttgaccagtactggctccaggggcgattatctcagcctgggaggccagggagtaatgcattgattagaatgtctctggacacatggattaaaatatctgatattttagggtggttgatagtggggaggacttctgaaactttttccctcttctatgcatttccatcctaatttgatttcattcaggatcaaaaaagaagatggcttttgaaattacatcccagaaaatctgaaactgtggcattgacttgctccagagagggctgcttgcatggaagaccttttcataggctcatcgtggaatagggacagatgataaagtttcttgggcatatgaaggggtcccagatttctggacgtcagatccacccatagatgattccttggattaaatgatgtgtgtgtgtatgtgtgtgtgtgtgtgtgtgtgtgtgtgtgtgtgtgtttaaagttttaaagtctcctgaaaattaccaagtgctactgaacattttttttgtcagtagtggatatctggataatttctttcaaggccacattgcttagcatgtataaggaaagtgtgtgcggcagagacccagatggacagccggcatgccatccagttgcttggggagtgtgaatactccttggcaaagccaaagggagccaaagaggacctctagtgtctttgctctcccatgtcctgactattccaatctcactttgcattttgagatcttttaacttttgctaacagtgattgctcaggatgtcattcaggccaattaaatttgaatgtctagggttgggacagaagctcacgaggtgaatgcaatgtgcagccacgttgagaaccactagctgacactgctaatctactctgctctcctccctgccttttggcggctttgccatgatatctgtcccatcctcacctcagtttgctgagaactcctcacttagttaaggaagttcttctaaggatgttcaactaatatgggctaaggcctcctatcccctaaaaatccagcatttgcctgagaaattggacgctaggaggataagacaggttcttagcagattctgaagcactcaccgtctctcatctgctgaagggtgtattgaggatgaatgatggggaacagcagggagaaagaccagcatccatagggcagtaacagggtgcagacacctatttatatgtgtcatcgtagttcccctgacagctcccagaaccagatattataccaggcagatataccaggcttacaggcacctatttatatgtgtcatctccttagttcccccaacaactcacagaggcagatattataccaggcttgagggcacaacaactcacagaggcagatattataccagacttgagggcacagtttgcataatgcattcataatgcaaagtggtttgtgatcttggaatgatcagacttcctacactggttctccccagtaatttgcatatcagccagataccctcctctagggttagctatgatcagggctgctgatttctggcactgatggctcaatgggaaggaaagcgcctatcccctctgttctgtgggattcagcaatcttgggctggtccttccaggaggcccaacctgaggacatgctttaaccaacaggcctttatattgagaaaatagtagttctcttccttgccatttccctgttaggaagccatggcttgcaggacagccagggagtagaggttcagagaaggaggctaaaaaaatcaagacctgaaaaagttaagtttccaaccatatattcccaaattgtagagtggaccatatggaatcattgcaatccctgggtttctcatttacaacttttgcaaaagatattactatgtatgctgtcaggcatgctggagccggatgtctagcacccattgctaagtgcatgcttcggtaagagctttggacttgggattctccagactgctgagtgacctacgtgtagtttagcaacataatgcttggaatgagccacgccgacctgatgcgagaaccggatggtagccgaagagagcaggccaggtagtgagacaactatgatctgcagaggtgggcagcggggtggagacaggcttcttctgcaccaagtactgctacctgaactgtacatcacagagacaagccctgccgagagcagtggaagcaggaaaacacaggcctatctctgtgtctgtcatgtaagtaagtctctcttggctgtgtgtacctgccaccatcaccccatcccaccccaaatatacagcatcaccttcagctatagcttttggtttttgattaaaggacagtaatatttccaggagggagaagagtgatgaacagaagcatttatgtatggacactgggcaatttattgtttttcaaagtctaatctagaacatgtttgcaagaaagcgtgaattgagtaaaaagtttactatcgtattaaggactgttaggtacaatgactgaaggaggagttaagagtgatccttcaataattcccctgtgggattatgcacatttaaggaaaaatgttcttcaggctatttctgccttagagctaggcatcattacagcgaaatagagaactaacctttaagcaagaggaaccaagttctagttctatttctgtcaatcactgtacgcacaacctctcaaagccttagtttcctctttgcaaagtgggataataaaccctacctacttaacacaatgtggggtttcaggtgagatgatgcataggatcgtgcttggcaagctgtaaatctgtaaattacaaatatatattatggtttcaactggtacattcctaagcgaatagcacattgctctgttgggaagacggctcttctccaagtcaggctgggataatgttccctgacaagacactgccatacctaggtgttccccaaacattgtctctgggaaccttgaggaagcaccataagacatgggaagaaatgttacagcgctggcttgaaagaataacaatgtatcagtctacttatttctgataatgtcatcttgggataagagactcagggtagcttagtgagggacatgggcatgcactgcacagtaaaaatggtgtccaggaaacctgggtttatttcagtatgggttgcccacacttctgccaacccagtcccctacttcgtccccagctgctcttgatgaactctctgcacacacttgcacctgtatctgtgaaacagagctcctcctcttacatgagaatggatctggttgcaaatctaatagattccgctaccacaatgtcccctgccttttttgtttacttcatttatgaaaatacccttgaaacatccatagtcccattttgtagacatggtgctttatgtctttgagattattaaatactcatgctcctttctgattgctgttttcacctcttctttaggcttgggcttttctattggtggaatttgctgttccttttcatggtgctggctttcctgagttgggtggttgctaattcttattgccagtttgtcttctgtgacagattctccgacatgcctcggatgtggcttccgtgcttggctttagctttttatctgggctctcgtgtcctgaatatttaccttctttcaggaatttctcacagcttctagccaatgaaagtcccccttcttattgtcaaacccagacagttataattttattttaaatgtactttttgttacattgttttgggagtagggctggaaggacttgctaatggatgagtggtggaaagtgagagaaagaaagatatgatggaagaaggagcaatcaaagccccagaaggaagctatcgcattgttcttggattcctaagcctgccagaaagagctgacttactttacagtttctgagagaactatgtgtgctattactagaagcacaagagaaagaaaaataggatgttcagcatgcttcatttatctaatgtgaaaaatgaactctgcccagtgacttaatgggcataaactctgtttctaaaaaagccactcattcggcaacgcacttctgagttcttgatatggtaaggtattgtgttctgtgctggacaggaatacaaaaatgcacggttcctcacctccaagaacttatagtacatgtagggaaataagacaacccctattgaatatcactcaagatggaaatgactagggccatggaatgtaacacacagagggtacctggagttcctaagacttctgatggatgattgaggagagccctggatcaggtaagcaatttgaagggatagggatagcacagacagcacaggggtggaaacaagtgtggagtgtcgagagcttgaccagtacgcctgaaagggagggagtgtacacagagcgttaataggagctctgtctcgaggcagcttccctcagcccctcccaggacatcgaggttttgggagaaagagcctattgctcactctcacggctcttctcctttttctctgctttcagtttgttctttgaactttttggaaacttcccctgttctttctttaacactgtgcttcatctcttggggttctacgttttgcaggttgtagtgcttgagatccagccttcccaaatgatttctctgaatttagtatttggtatgggttttgctattttgctgccatcccagccctagcaaagaaacgacttacccggagtatggacagggcttcagagaaaacccctaacattcctgactcccgactttacagagctctgccaaaccttgccttgcgggagtaagaaaagcgctaacaagccatcctctttggtgtcaagtgcagacaaatcacttagcccctctgaggtcctccaacagtaagctactggtttgtgaaaccccaggataatccatctgatttcagtcctgcatttagtcacttagaacattctcgcacatgcatttgctggctcatgtacatacgaatatacacatatccctctttgtgccctttcttagcctctgatgatttcttctcctccaggaggcaggaatccaaggcttataaaccatgacttctgggaagttttttcttctgcttaaccagggtatcattgttttttctgccttcccctggagaatcactggccactgccctagtggttggggcaaggatcagaggtagcttgcattctggggtttgtccccaaagcctcggtgggactctgcattggggtctgtagcctggatccactccagtaccttaactaatctcttgactcccagatggtccaaaatatgtggatttagaagagcaacagacagctgttcctctgggcctctccaagaacacggtttggtgtctagaccaccttagagaaacatggcagaggaaatcatggtggagcagcatggaaacaggtgaaacccagacttagtaccttgttaaattccatcctggagtggagataccagaggagcagatattacctttattaactgatagaaatgtttggggatttctctgacttcttgtagggttggataagccccaaagtgaagagaattttgctccttgttttagccattaggaaactcaagaccctgctacagtgctattggtttaatttttccctatcacattgcctctgcaacttctgaatggttgcagccatttcttaaaatttccctgcattgtcactcagacaacaagaatagatttggccttcttcatctcaaaataatggtcatgattaatagttattggactgggaacagtgctcagccctctgtacgtgatctcaggaatcctcacagtactcaatgaaatagcaattttattatctcatttttgcagacaaagcaacggaaacttccacacattttctacattgcacctaagatcatctgagaaactatgctgtacttgtttttctaatgtatgatctgatttttctattataatgttaattctatgaggacagggctttctgtggccttgcttcattgctgtatctccagcacctggactactgcatggcacctggtagttacttagtaaaggtttttcaaatgactgagtaactcatccaagattaaatgtctaggaagtggtggcaccaagcttaggacgactcttttctgattccagagtccagacagccctaaccactatcccacactaccttcttgtttatttttaaatcattttccttcccttcaatccctctccagtgccttacaccttcttgctgtaatttgaagcatggccacagtaagctacctcaagtttctcatctgtaaaatggggataatataatgaactaccttatgggattgtacccctctgcatggtagcctcatcctactgtgcctcctaaccacggcctttaaatcagcaggtatagttaatatatttagttcttttaatctaatctgaaacacaaagcatttgcttccttaattcaagatttttggctttgcctagactaagcttaaaaccaaagaagtactgcagaactgactgaggctgccagaagtaccacactcttgcacccagccagtgggaagtggaaagataacagctaagcctttggggatccttccagaagtagtgatgacgtacagcattctttctgattatgaagtaaatatctgttctaatgtatgttcaacatagagagttaagaaaatggggaaagaataaagagtaaaacaatgaccagaaataccttcaataccctttgacattctttctctgtgtgtgcatgtgtttgtgtgtctttgtttctgtgtctgtatatgtgtatttcctttatttttgtttttttactttaatgtaatttttagagacaaggtcttactttgtcttgtagactcgcgtgtggtggcgtggcactcatggttcactgcaacctccaattcctgggctcaagcgatcctcttacctcagcctcctgtgtagttaggactgcaggcatgcatcaccattcctggctaatttttcaatttttgtgaagacgggctctcactatgttactcaggatggtctcaaactcctggcccgaagcaatcctatcaccttggcctcccaaagtgctgggattacaggcgtgagcgaccatgcccagctcccttttataaataaggggctcaccatacaatataaccagtttttacctggcattttccagtcattattgcattgtacgtatctccccatgtctttttcttttctttttttttttttttttgttgacggagtctcactctcttgccgggctggaaggcagtggcgcaatctcagctcactgcaacctccgcctcccgggttcaagtgattctcctgcctcagcctcccgagtagctgggactacaggcgcccgccaccacgcccagctaatttttgtatttttagtagagacggggtttcaccatgtcagcccaggaaggtctcgatctcttgacctcgtgatccacccgcctcggcttcccaaagtgctggaattacaggcgtgagccaccgcgcctggccctctccatgtctttaattattcttgcataagatgacttttcactgcataatattccatcacataccactctttaaccattttgcttctggggcacattttccttttggtcacactttttatactacagttgccatccttttacatacatttgaatacacatatctggctattctctcagaatagatttccagacgttacctttccaagcttgaagatgttaacattttaaagaaagatggttattcttgaaagccctgacagctctgagtggggagccggggctgatggttaccacaggatagcggaaaggcacactggctggcctgtgtgtactcacgcatccccccacctagggcagccttgggaagagcactcaggattatgagaaagactgtcgcctcccctttgcttcattagctgatcctctaagcatatgtgctttcttggtctaattttcggattggtcttctcctatattctcttcctactccccaccccgaccttacagctaagtgcacatctcatgtagtgcagtgggaaagaaccgtaaggcagaagccgggctgacttggctgtgaatcccagctccatcacttgctggccaggtgactgagtaagatcgtttacacatccatcatcctcaagtttctcatctgtaaaatggggataataatgtaactgccttatggcattatataaggattgtatgactgaacacatgtagaatgcttagaacaatgcctggcatatatgaagcatttaatacatggtgtattaaattagttttgaaaagaataaattaataacaatgatgaacatttttgatacctattttcctattgttttgactctcaaagccagttgcaagcatatttagcactgtgatgtatgtgtgacttactgcaaagtcttttttccagtccctgataccagctctctcttcaccttcagtgtttcctacccctcctgcctccccttccctaagaatattgctgtttcacagagtgtaggctttcctctggcttccagatctgcccacatatgcacacttctctttcccatccctgttggactctttctccttatcagtttatttgttccagttgggaagaactggaacctggtcggcagcttttccagttggctttatctgtgcgctgcattgtaaaactgttctctcttgcttagaaatctctttgatccatgtttagctgtatttattcttccaacagatgttttgggtagtgagaggattttcttctcgcatttgcctagtctcatgctccttcatgcttcccacttgttcgggatctttttgccagctgaccacagacaggggccatctgtcgtgaaggtctccctggcccagcagaccaggaatggcccagcaaccaagactttctgaagggcttagtgaaggggaggagggaggaagatgttggagaactgtgtagggtagagtttgagtttcccagacacattccaggagctcttttgatccaaggtatacatgatttggcttgtgctctgtggcaggttaacaaaaacacaaccttccattgtctcctgtagacaacagagtgaggcccttgggcatggcaggtagcctaagactacccctgagagttgggaagtgtatgagtctcctggggctgccgtaacaaagcaccacaaattgggtggcttagcacaacagaaatgtattgcctcacagttctggaggccagaagtccaagatcaaggttgcggtcagggccgtgtttcatctgaaggcccagggaagcagctgccccacgccttctcctagcctctggtagcctctggcatttcttggcttatagatgcatctgtcaaatcctgtgtcttcatatggctttctcctttgtctcacactgtctttcctctgtgcatgtctgtgttcagatgtccctttttataaggatgtcaacccaattggattaagttctaccctaatgatctcattttaacttggttacctctgtaaagtccttatttccaaataaggtcatgtgctcaagtactaaggggttaggactccagcatatcttggtggtagacacaattcaacccataatgggaaggaaagatgttgggcacctgtaactcctccaaacacccacagagtgcagggtgagctgtgtgctaacacatagtcagttctctttggggtgaggaggcctaggggcagggcccccatgtggggtctctgtccacaccagcaacaataacaaccagggaggaaagcatctcattttccttggctcagttcagctttttatgtttttagcacaatgcctgctttgctcttccaacaatttgggaatctctgggagctgtgcatggaaagcaaggaggacagcggcgagaaaaagggggagtagatggagggtcttggaaagcagagggcctaggcaggcagagaggacaggaaagtatagcgagcagagcggcaaattggtggggaggtgcagaaggctgcttggcagccaggagttcttgccctggccctgccatgaggctgcatgtctgtggcctaggtatttaccttctccaggcctcagtttctctgtaggcaagattgggaggtggatgggtgctctctaggatcccttcctggccagaataacattctcagcaggagcctaacgtgtggagcaaatgggagcactgggctccggcctcctgcagtgagcacagcccctgttcttgtggaaacatcttccaatagggctgccctgcctacagggtcatgcggcatgcatctgctgcctgcctgcgctcttgaaacagcctccactgctcccctcccagctcctgtctctctgcacacgcaagcgtgctactccttttcatgatccccattagtattctttgacgatggcatacatctgtcttcgatcgttgtcagctctgggaggcttatgccaagcttcttgagcgtaacccatgactgcctgggttaggtgttgtgagctgtccaggaggcaggaggacgatgcatgcaagtcagggcttagggcagaagtgcctgggcctggcctccccttggactccaggagtcctgtcctaacagagcccacagccccctatccatctggcctctgtaacccctccccaacacacacacacacacacacacacacacacacacacacacacacacacacatagcccctgtgattgagggggccccaattcctgttcatatcctccaggatagcccacctgcaccctcgacagtgagagacaaagttctattccctgtttagatgggtgctggggacaatggaaaggaggtgtggctctgagaagttcatgtcttgctcagggcacacagcagctgatcgggaacatgttgctgactccaagatgctgccttgcaagaagctggctctatccttcttttggctgaagtgcctttcatggatggtgagggatgtgcagggagaagtgtcaggagtgagggtcagtggttagaatcaggcagtccacagagtctgagaaagcaagacattctctggcagtctgggggtcatgatcgcccaccccagcccagataaccctcacagctgtgcgggccactagagaaaaaggagggcatgtttggggcaggagaggcaaatgtttgcttatctgtgacttcttcctccaagcatgtccggacctccagtcaatggtgggctgtcagtcgtcagctgaggttgagctttccttagcaggagcactggtcacttgggctgggatggttcttagtggtacaggatgcactgcaagctttaaatgcgagtggcatcatccccttccggtcaccatggcaaccagaaacaccttgacacatttccaaatgccctttagtagggcagtgacagcccttttgagaatcacatagaatcgcattgattgatgagtgaaaaataaatggatggtagcctccttttgtgatttttgcagcggcctttagcttcctttactcaccccagaaatcagtgggaccctgggagctgtgtacccctcagacccagttggaacccagccaagagtacttaatccatccccacttgtggggccaacggcacctaaccacctcaggcacggtggacctggctcctcagagagctctagggacagaggagagaaagggtctgcattctgtttgcagccctgatcgtgagctctgggggtcctcttccacccccacccccacccccagcccctggagcaggtactcggggtcagagctctgctgagggtctggctctgggaggggaggtttgtgtaagattccctcccacggttcagcacagatgggatgacaaggaccaaattctgtttctgggctctgatatttgccaagatttttaccaggcttcctggaatagacagggaagcagagcaagctcccgtaggtcaagtgatttgggcccgagttgacccagagtccctaaatgactgctgtgtagctaccatgagtgtgctgagtggcccataggggcaggtatgagagaggtgctgagggaggcagggggcccgcagaacggcctcccatctccactgcccgtccccaggtccacaggctcacagagcagacacggtctgtgcctgggtttgctcacccacaagaggaagaacataacatctcgctccttttgctgcacaggataaaacgagaacagagaggaaacaggaagtgctttgcattccagaaagagcagcaactgtataaagtcatgcatattaggatttgaggtatgcatggtcagaagttagaaactaaccgaatcttgtcattgccaggaagtttcggggttctgtgactggtggccactgatgttcctgtgttcctccattccagctcctaccttgactgtgtcctcctcttcacacctaacttctttagtgaaggctccatttcctcatttcctgttcaatgcttaaaccccttgcaatctggcttctaccatcgccttatcacagacccttctctggctttgccttgcctggcccttccatgatgtcttctttcttgaaaccctctttccttggtaggataccacggcatcctggtttttgtcctacctctgtggctgtttctgcgtgctttccttagctgacttttgctcctttatctgacctgggctctcctctctccatacactctccatagcctattctaagtgtcccaggtctcttatatcttatctctcaaatgcacaattactttgtgttagctacagacccatatatccagcttccctatagataccccaaatgtctttgtaggctcctaaactcagtgtatgctaagctgaacaggggctccttttgtgccccaaactttcccaactccagtgagtcttctccattgtctttcaccttaataaatggaaccacctgcgactctagtgtgtggtccagagacttggaagtcacctcagcttgtctctctgtcatccgcaggatcgggcagcctccaagtcctcatcattctaactctcataatgcctctggagtttgtccagatctcctcgtcaccactgccgctacgctaatcaaaaccaccattgtctcttgccatcctccatactttgcaaagttaattgggtcatttttctacttgaaatcttataatggctctccagtgcctctgagtccttgttttttcaacactgttcacactctccccacctctctctcactcataccccatgcaccagccatcctgggttttgctgtttttgtttcccagaatgcacaatgcaccttctggcctctgagccacagcacctgggtatttgctcacactactgcagctctcctcttccctgccaccacgcctttcttgcctgactgttagtatgcagcagtggccacttgagcatgaccgcctctgggaggctgtccctagtcctctgtcgcattctggggctccctatcacacactcccattgcatgctgcagcatcctcagcacccagcattccttattgtagttcctgattcaacacctttctcaggagactctggacttcttgaaggcaggaacaattcccacttgttcctagtagcattccaaaccacacgtgacagtgtctggcttataataagcagccaataaaaagttgatgaatgaatgaataagtgaaaacagaaggtgtttgcctgcagaaatctggaataagatcaaagatcagagctgggattaaggaaaaaacttccttggggtggcactatgaattcccagaacaggtgactaaccctcattcactttggcaaatgtttatcccatgccacgcaaccaggcaaaaagttgaatgaggtttaatcccttcccacacggagcttattccttctttggaagtcctttaaacaagctctgaaatgattttggcaggtagacaaactggtcctcatttctctgtgaccagtaagtagggaaagcaagcacacatacacacacacacacacacgtgcacacgcacactgacagacaaccttgctcactcacatgggcatgcccaaacccttcttctattttataggatggtaactcactctttagtttagactcttgacgtgccatggaaaatcccactcgccctagaactgggggccgggcaggtttgactgtaacaacgaagcctggagcttactctttgctgattggctttcctttctgtctccatttttcccctggtgagcactgcagttgtgttcttcctcccaaaggtaatgcctggtttggctcactaaaacctgttctttctgtaccgagagctcatcttctcttcctcttctggattctcaaatgagatgacgtcagaggatggaggccaaccacacccttcctccttgaccctgataaagtttcttggaaaccctatactcagaggcagccaattcttgccagtggaagagtgaaaagagggcttgggaagctcaaggctcagtgtctgtccccaggtcccccagttaaagacacatctgtccttcactctcaaagatgttgccattgctcccctgctagagtgacacactgcactccctccttcccttcacaccccagcaagaggctatttcccaggggtcttataagcagatttcatcttctcttgtgctgttttcttatttcaattatcttcagggaggaacgtgcatattgcgtcattgcctggctgtgaaatttcatttccatttctttacacctgcagttgcaatacgagagagaaaaggccagagcttagcggatgtcctagacgcaggttatcaaggtgctgtggctgtggtttcccggaaaaggccttggtcccagagcacattttatcagcaggaccttcgaggggctgcgttccttcaattgttttctctttggggtctctggtctccagttctttcttctctagcatgtgagatctgtgcttttgattcatgcctttaagtctgacattgaaaaaatatctgatttgccattccagatgctcgtcctcatttgcaaattttcctaaagggccagattgtcctctggccttttcccttttcctggtcccacctcaccaccctcccactggggcttcacagaggcagagctagtctcctttcattttttaaaattaatagtcttcaatttttagaacagttttaagttcacagaaaaattaaccagctattacagagttaccgtataactcctccccctcactccccagttttctccattattagcatgttgcattagtgcagtacatttgttgcagttaataagcaaatattagcccatcattattaactcaagtctatagtttacattaaggtgtattctttgtgttttacagttttatgggttatgacaaatgcataatgttatgtatccaccattatagcatacagaatagtttcactgccctaaaactctcctgtgctccacctgcccatccctcctccctcctctgccaccaatccctggcagccaccagtcttttgactgcctagagtttcgcctttttcagaatatcatagtagttggaataatacagtgcgtagtcttttcagactggctcctttcacttagcaatatgcattttaagtttcttccatggaaactttgctttcatccttttatcaccacaaggccagtcatccaaggaatttctccatctctgtctgttcctttctagttctatgtgtgccactgcttggcatagaataggtatccatttaatgaacattccctttcaccacctgggacaccttcccagggataacaaaaataaaaccagctaggtcaatagcagagcccccatcccagttttaacctcattctcccctctttccacaataaactggatcagaaccagcagctctgtaagactgcatttctttcccttaataccaggccccagagagcatttgattccttggcagagaggtgtaggcttaattaatttttctccttttttctttgaacatcttggaacacacacacacacattcgcatttatgcacaattgggtgtatgagaattttaatggcaggtggtgttagcagttcttttcctcctgatacagatcagggtttttccatctgggccttttagcagggcctatgaatattgactttctaaccacttggatttgggtggagtgtgcagagtgggattgggggggaaggttcaagggagaactatacttatgtataaatcacatgtgaagggagttttgaagtcattattgcttcaggatgtgcgaaccataattattttttaaggtcttgatttgcccaaagagcatttcccagggttgctgctccaagcatgacgtctgtgctgtcaggaggtgcagcatagtctgattcgagtttaatcgctttaaaggaggccctgggtaggatctggtctctaggttctcagctgtggtcagtcctccatgcagcaaaacatccagatgacttagatgattaagacagcagacttaaagtgaagaagagattttttcccttattctttccttttattattagtttttaaatggttggctacatgggctgttggtcattctccatgttctctgtgctctcctcagctctctgctcaaaacaggctgcaccggcctgcctaaaccctgaaagcaacttctcagctgcctactttctgccttttgacccccaagccaatccccatctccttaccaccctcccgccatgtcctcatacacctgcctctccttgacttcattcttcatgctccatcagcaacagccctctgtcaataatgattgtcccagggaagtgtattcaagggtcacataaaatgtgccctctctatgtgttgagaaggttttctgtccccaaaggagctctctggataatgaggaaggttgaactggggcagcctacaggaagaagcccttagaagggaaacctgtggcataaaccatgctgatccacgactcttattttggaatagctatttaaaaagaaatatgaagaactcgtaagacttggaaaagaaaactagagaatgttgaaaatgtccaagggttatgtgtatgatgtgtatggggaaattttaaaagaatgtggtagaaaactgaatttgtggtaaaatgttgtcacaggacggcctgttctttcattgaattatgtcttagtgcagggatgacaaataaatacagcaactgtgctgccattctcacatcttttcctacagtaggcatcactaatcaattacaacattcttttccactgtcatgcctttgcctacagcagacatcactaatcaatcacagcactctctctcattgagtttcaaaggttttttaatcctccacataggtctctaagtagctatataccaattactttgatttagaacttggattaccaatttgccatctctagtttaatgggaagaacattaggattagagtcagaatacctgaattcaagttactgacctactacttaataataattaattttaataattctctaacctcagctttctcctctataataagaaataatgcttaccacagaggctcgaggtaaggattaagtaagataatatataacatgtaagataatttgtgtgatggttcctagaatagtacctggtgcattgtaagcacccagcaagtgatagccaagtatgaatttgcagcggcatggacacatcatgccacaccccagatatggaacaatagttaggattccattcacctttgcttcttttaacatcctcagtgaaggcagatggagaacagctggaccctctaattctacctgactttaacttcacattcttaagatgcttattaaatctctctttcctgaactaatataatgcttccttgttaccctggaaggacagagttaacacccatgtattatgacatgatggatgttctttttgcgcacactgcatgcatagccatgtgccaaggccaggcctgccttagctttttggttctccagagcagcttcactgtgactgaagagagccagggaagacatcttggtagagctctttatatgactcttttcagaatgtttctcactgatggagatgacagaaagctaggatgatttgcaggcaggaggacaggcttctttggaaaaggtttcaccatagattacctcaatccagggttggaagactggaaagtggtctgcagggcatcccaggggatgccttcttcaagacaatattagactagcattggaccctgcctccagactagtaaagggttattttcagagcagcaacaccagagaacatgttttgagcaaatcaaatccttaaaatcatgatttcatacatcctgagatactagtgacttcaaaatgcctttctcaaattatacatatagtgccttctgaatcaactgtcttttttctgtccaacagtataaacataatctctgccctcacacagcttagaaactgtcatgagaggtaaacaaatacacaaatgacaaggtaaaatagaagagctacaaaagagatcatgaaaatatccagggagttcgaaggtggcagataacaaaaggcttcgtggaggaggtagcctttgtggcatttgagatcagtcctgcagcagggttataggtggacaggcagagatggttgggggtgggcggtaggagggacagacagaaggaaccacaagaatgaggatatggagaaaaagaccactgaagggtgcaaattattaaggatggatttatgtaaaggatgtggcctaagaagaaggactcttctttatgatgaaattaggttttagaggcagaagtctggtgcttcccggtactaaaatgaccccagtatggtgtctgcagaatacttagataactgcatagatggttcagccttctttccattataccacactactgggttaccaacttgctgtgtgaccttagataagcaactacctctctctgggcctcagttttcatatctatgaaattaggaggttgaattatgtcttggcataataacgaaataaataagtgatttatttttatttctttgtcattcctctttgaaatgggagtgggaataaagtggtatttgtttccacatgaaaattaaagccagggggccaattctggacttggcttgagctgtgctttgtctgtttttctgcactggcccagtaccctttacactggtgtaactaactccctggaaagggatgcaggtagacagcgtgactgttccttcctatctgagaggccccaacagattctatattgcaggaccaccaacttggaatttcagctcagttcagcaaacacttttgactggttcgtgtgctagacattgtgctgggcttatgtggggtagagatgcctgctaatctaaggctaacaggaagacagggaagtggtttccagacctgatgcttatcagagtcactgtggaagaactttttagaatgcagacagcccccaacttaacagtggttggactttcaatttttcaccttataatggtgcaaaagtgatgggcattcagtatgcttctcaacatacaaagaggttgtgtgaaataaacccatcattaattgaagatactgtcaatcaaaaatgtacttttgacttacagtgttttcaacttaggaggggtcataggttgaggagcatctccacagattcctgagccccatcccttgatgatgatgatccagtgagcctgggtggggcctgagaatctgcatactaactgctgaaggtgagtgtgatgcagggccaagcttcagaacctctgatctagagaagattctgtgtcatcacagctcagggtgaccatgttctctcttacttgcacttacatgaattcatatgaattcagtattacatgagtaaattatgattatatgaatttattgagtatcctatgtgcttgatataggtgtttacatcccagggttggggggtagagatgaagaatataagccggactatgttaagatcttcacttctcagcacctgaacaactggcagcctcattaggaaggcaagtcattcacaggtgaaatgacatcatggtcgtcttcttcattcttctcttcagcatccatttaagactcacattttatcaccagagactgaaaagagccacctaaggcaggcaggtcaggtggtgttatctctttatttccagatgtggaggctgaggctcagagaggtgaatccatatgtccaagctcacatctcccgccctcagtccagggcttctccccacttcatgggagaagcatcctcctccccagagcagtaggttctggagctgggagaggccactgtgggctggattgttggggacagcttcagggagagcccgattcaaggcaatagagaactttggctgcaggccgttgcctagaatagggcagctgacacacctttgatctggaatgattcctgctgctgagaatgaggttttttatatctggattctcaggtagtaacaccacgacaacgtgtgtttgtgttatttcatctgcacagcattcacgtgtagcagagagaaggtagtttattccccagaagtttaccagtgggaaattgaggtcaaaggtaggactttcctaaatgaagagaactactatttattggatgcctcccatctgccaagcagtgtgcacagcaggcatatatcatgaaatgcctcatttattcctcataactgtagcctgattttacattcggcaaacctgaggcttacagaaatcatgtggctcgcactcagactgctgatggccacgcaaggctttcagcttctctgatttccaggcccccatccccaccacactgtgctgtccagctcacctggtggaactggatcctttgagttccaggccagggatcctactgagctcttccatcagggaaaccatagcagtagcagctccaccaaggacttggcatctatcttactgcagcatccgtgcctgtctaatggaaccatgtaccaggagtagctacccaagaaacatttccaccagaaacttctctttatagctccccatcgagcctcagagagctgataggaattgctcaagaccacccaacttgtaagtggtagagctgggactaaaaggcaagtcccccaactttcagcctcactcgtgcccaatgtgtctcagcctccctgaagaatatcagaccaagatggccagaaaaggaacctggaagggacgtgtgggcgtcatgcagcccttggcacagtcttcaggctgagctgcctccaccttgtcatctcatcagagctttacatccatccttgggagaggccagctccatgacctctcagtgtcatttaggatcttcttcctaaacggctaacccaagtctgtccgctgcatcctacagtgagatgcactgtggcgagagcagctattggcattctgcttacgtgctgtttccagaggtaaactcagtataaatggatctacagcctgtcccattttgtacagtcgagttctaaaaccagcctgcaaggataatgctataaaaatgtcctgccagccccaagggtgtcttctacaacaagttcttgttccctcataattcttctgacaaattcttcttttgatccagactttcctagccatcatttcattccagaagtggcatgtgtgtgcaaacactcatgcttgagggcgggacaagcaaagggataagaggggaaatgggaactcgaaatctgctcaaatgtggtaaagaaatatatccagaaagtactgcctactcaccaaaatactatttgctttttatattctttcctgagtagaatttcctgttcaaacttgaaaatgaaaattcctccacttcaaaatgaacaggcaagaaatgctgtaggctgggtttcccggggaggggggactgacagccatctgccctgagactgactgtcaaatctgaactctgtgtacttgttagtgttgtttatgggaggggtgagggaagggaggaagcaacagggacctgctaaccctatgaattctccctcatacccttaaaaagtcgggtgcttggccgggtgcagtggctcacgcctgtaattccaacacttgggaggccagggcggttggatcacaaggtcaggagttcaagaccagcctggccaagatggtgaaaccccgtctctactaaaaatacaaaaattagccaggcgtactggcaggcgccggtaatcccagctactcgggaggctgaggcagagaattgcttgaacccaggaagtgaaggttgcaatgagccgagatcatgccactgcactccaacctgtgcaacagagcgagactccgtctcaaaaaaaaaaaaaaaaaaaaaaaaaaaaaaaaaaagtcgggggcttgccaagctcgctttttggggcaatgggagggaaattttaagagctgatttctgtgatctttatcagacctcttcttctgtcctgcccacaggtcagtgatgggaatgatagtattgcattaaaagagaagtgattccccagatgtggacatctcagtgcctgggaggcagacgtctttagagtgcttggtgttcctccccatatctaacttacctgttcttaggacatattcccttgagtgtcttttattatttcgttgattgattaagggttacaaatttgttacaaaagcagcagaaattttgaaagataaaacaggtagaaactgttatttaattggaggcacaaaagccagttccagcttgagcattaactggctgtgtggcttgggcagctcccctgcacttttgggaccatggtttcttcagctctaaagtgaaagctgagagttcctctgaatgctaaaatcttgcttctctgtgattattgttttttaaatgccgccgtcatttcaaaacacatacatggggcagccctcctcagctgcctgtcctgttctcttccctccttccctttgtgccatcagcgtctcccctggatttatcgtgctgtactccttggacacccttgaaagtggagagagaataattctcattccctttccccaaactctgtcctgaagcaccactccccacctccctaccatgcctgcccctccaccccccacattttccctctgatgagatttcctctctgagggaatattttggtcctgttctctgttcccaaacaaattccccaggtcctttaaatctggtttcaatcaacataatcacagtctcttcgatgctatcaactctgccttgatttggttgtgtgattcatgctgtgtcctccacctcctcaggctgttttatatatacacacttaagtgctcatatatatgtgtatatattacacaggttgtatgtatatgatgtattgaaagagagagatgttttccaccgtgcctgtaattactttttatcttcctccaaatcagtatcttaatgcatatcacctaaactggattcattttgagaacactagggccttttttttacttttcaagaacagctatctaattctgggccaaaaactagttatcaaatgagggcaggtaatggagttgtatccagcgagggggcatttgcttctccatatgatacactcctcaccagtaccaaggcgttttctcttcagtcttcatgcctatgctgttacgagtcctttctcctatttgaaagaaaagatatgaggcagcccagggaaagctctgatggaggctataaagacaatatggatgtaaactaaaaaatggtagcttagagcttaatggtagcctctcaggacactcaccacacctgaattctaccagcctgacgccaatgctgctccgcttatgctgggatgtagtgacacaggacactctgctgggggatagggatgatctccccccaccaagtgggtcatcaacactcatcactcagaggggaggtgggataccaccttgaacagagaaagcggctgggcatggtggctcacacctgtagtcccagcaccttgggagcccgaggcaggtggatcgctttagcccaggagtaagagaccagcctgggcaacatggcaaaaccccatcttcattaaaaatagaaacattagcctggcttggggttgcacacctatagtcccagctactggggaggctgaggtgggaggatcgcttgagcctagggggtcgaggctgcagtgagccatgattgtgccattgaactcctgcctaggagacagagtgagatcctgtctcaaaaaaaaaaagaggaagaagttgctgaatccgtcttataaatctgtaacagaaaattatggcaagttgtggcttttaattgccagagctgggcacttaagagagaaaagggtttttgtgaattccaaaaatgtaattgtattcattgaacactgaagtcaggagaagtcagaccataatgacatgggagccattttcgcaaccagcagactagaaggggagggtttggaagggtggcggcagagcttagacacctgctgcgaagggagagaagtggggacagaaagatggcctgtgtagagtcccatgagaaagacagaactgcactggcaggcatctttaggggcccaggtcacaatcatggggccggtggacagtctccagggcactgtgattgtcacagtgcacagcctaatggggaaaattgcacagcttcacttaaatataggtgacatatggacgtaagaattcatgattaatctagaactaaccctgaccagcaaggccgaatgaagaaaaaatgtagaaacactgaccaaaccttcctcaaaggtcacagatcttagggaatgtgtctctctttcacatttcaaaataacaacaatttttgaaatatgtattttttaacatttaattgttttatttggggataattttagactcatggcatccactgtttttttagttacccacatatttgattcagtaagattccctgtattgatacaaaaaaagaagagggaccctgtttccctttgacactgttactttttcctgagctcattgtttgtttgtattttgttttgttttggtttggtttttgagatgtagtcttgctctgtcacccaggctagagtgcaatggcgcgatctcagctcactgcaagctccgcctcccgggttcactccattctcctacctcagcctcctgagtagctgggactacaggtgcccgccaccatgcctggctaattttttgtatttttagtagagatggggattcacggtgttagccatgatggtcttgatctcctgacctcgtgatccacccgcctcggcctcccaaagtgctgggattacaggcgtgagccactgcgctcggcctcctgagctcattttaagagagacttctggcctagaggtttgaatgagaagaagttatacagctgggattcttccctttctctgatatgaggacaggagttctctctcatctccgccaagagcaggaagctggagtaactgccacaagctccaggagggagtgtctagaacatccacgttttgcagcaggaaaacaccccctcacgctgaagtttgattcctgaatcctgtgtcgcagtctaaatgctgaggcagaaggggacatccgtgttcctggggcattccacttgcagtcctggctgtaacccgagtgagccatccgtgtagttcctgttgctaagtctcccctgccacctcttcttcccatggctgcagggcagggggccatgccctcctcttcatttcctgtccctgggtgagcgtgccccctgccttctcccagatctctgctgtggcagcttcacgtgggattcagcactgtgtctccttcccctctgctcggcctgcccatacctgtccagcagagctgtaagaccagaagacagagcattccccttatctatgaagtcaaatgcatgtgtggaacatgccacccagcctgcagtctctctactataaaatactgctcataagacaaatgtgtggcccagatgatttctgataaagtctattattttgaaatacatatgtatgtctctcagccactgatacacgcagaagctgcacatgttgtcaacacctgctttgagctcctttccttcccacccttccttttggcaatgcaagtttccattcattttctgcattactggtctcctctcttctcccctactactagatcttacaataaacatttgaaatagtttatttgtccacagtgtgatttctgttgagaaacatgggctcaccgacttttgggtctcttctaacactagaaattcctctggttttttagactatttcaagggctattaatgtggaacagacggcctttagaaacagcaatccacagggggcccggagacctggattcatatttctcctagcgtcaactagtgggtgaccttagaaatgtcattttccttgtcgggctttagttcccttatctgtcacacagaagcactgtgtgagtttgggaaaccaatactatgttgaaatgtacaaaataatcttaaagcacagatattctgttccctccaagaatacatcaaacaaaggaactgacattgcaagaagattttgaggagatggctggatgcactggagcagggattgctgagggaagccaggccctcacctggagcgtctcaggagaggcagcttcggtgctggctgtttattgcaggcatctctttctgtgtctgtgcgtttaggggctcttctttggagataagaaaagggttctggatggagggcagtgaagaacagtgagaacttaacatgaggatgtttgtatagaggggaagacttctggacagtggcttgactttgctcactgggcatttccttctggatctctgtagaagtcagggacagatctcctcgtgcatatctgtctcccagagacagatctcttcctagcagaaagtagaaagtgggcttcaggcatcctggaagttttctttcttggtgggtgataaaagggcttgcagagagaggagaatcaaatctcccacatgtgcatcatgcctgcgagtctcatgcagagatgtcttatgttcaacatagaaagcaagcctggcagccccaagaccttcctctgcacaccgtccatttttacttggtttcattttgataactgtgcggtctgaggtcctggccaagaaagcatcacctggcaagaagtgtatttggccaatggtaaggttaccatctctgtgtaattaggctccgtaaagctttgtttttaaatttattaatgggaatgatttgacattcctacacactgacattaccctcatggaatggataagaatctcaaggcttgttgggtgaaagaagggcagtgtttggtgtgacgggaagggaaagtataagcaggcagctcgtgcgcatgagcatttgggaaacagaacagaaatcatagaatggcaggcttaattctagctctgtcacctactggctgtttgtcattagaaaaattatttacccttcgtgagattcagtttccttacatttaaaataaagaaaatattcgtcctcatattgaaatgaattgggctatcatgatcaactttaaaatacaacgaacagtataaatgtcaggaattatatgacattcgggacctccactgccaccctcactttctccctccagtggtcacttactctctgtccctctttctgggtcagagcttctgttactccagcctgggcctgccttaagtggggacatgtgctgatccctcacaatgccgggtgacaaggagggttttcaaggctggcttgactgccactgctggtctctcttctcatttgcaactgtcttctccccgctgggctcagtgttcctgggagggtgatgctgagggagaaagctgtggcagagggacgtggcagggtcagagaccactgattcgcaggagctggcctcagagctagcctttttgcattgatctagggaaccagtgatcatagatatctatgttgacgcctgtgtcaatttatccctagagccattattcagtgaatttcctaaggggaaaacaattctccagcattatttttctttgggccaggagagcactttctctgagttttactggcaagctagatatattcttgaaaggctccagcagcagagttcccgtcttgtttagttcaaaaacagctcctggcccgtctctaaatggtcttgctaaaaacatctcctcccacccatagacctgaacttaagcctcagactgctatcctctctttctgccactgtgagagacctatgcctcttttctcattggcttttgcctgcccagccctcctcagatccttgtacatccctaggaagtacattctttccactccacacacataattgtgcaacttgtgctaggaccgcatgaggcactggagctgcagatagcaaggaaatgtgggccctgccctcaggaacttgaaatccggtagttacctagagctacactgagttccctgacgtggtaggaagccctcccagagccttgtccgtgcttagacgttgccttcacagaggtggctaaggggcattttgtccctgccctagtttttacaagtcccctggatgttaactcctacttgcttttatttgcaggttctccagtcttatgcattctctcttattcctaaaaatttccaatccagtgctgatagtatgttagagctgtagggccaggaaaccctgctgggggaatcatcgtctagtaggtggagtgtgagagagaggagactcaggccagaggggcttctgagttctgggcaagtcccttaccatcctagtgcagctatccttctccatgtcctactgagctgccttccttcttgcttctcattcccaaagagaggcaaccatgccattctgggcagtgggcaagggctgctggaattgagatttttatttttctctttggcatctgaatccttcctttgcagttgctgacatgcagttttgtgtgagatcacccatgtcatccatcatcttcaggacacccagaaactcctcctacctctctcagcctcacatgcgcatcttcagtccccttctgattcaccctagttggtctattgctcttggctctgaataaatacctttaaagggtacaggtatccatatggaaggccttcagagaagagagagcatggaatttattttccaaagtgggacactcgagagtgaaagggtgagctgacaataatgatggcaggacaaaagcatatttcaggcttcttaagagcttcatccctttgatccagtgactttatttctaggaatcttttatggagtacctaccatgagccaggcaccattgcaggtgctaaggatatcaaaacacatcaaaagagagcatacagttaagtggagcagacaggcaagaaaaaggatggttgtaaaagaatgcggtaagtgctgagaagtctttctcagagacgtgatgtatgagcaggctcagacagtcaaatagtagttagccagggaaggaaagggagaaattgccttcctggcacagggagttgtcaggggagccctggcagaggtgagagtatacatggtgtgtttggaaaacccaggtccctcgctgaggctgctgtgcacagggagaggagggctggcaagagatgaggctggcaagtgaggcacccaggagccagacgatgaagggcagacaatgctgaggaattttagcttgatcttggaggttatagagaagaacagaaatttctccagctgaggaatgacccaattccattccagtgcggggaagaaatgatagggaaggctgggagagggcaaaatagggtctcggaaaccatttaagagtctattgaagtaatccaggcgaaagaggtagcatggactgtagagaaaaggagggtggattttgagaaataggaggaggcagagtcaactggaattattaaagatcagttcaatgtgggggcagggaagaaaggaagaaagaggactcaaacatgatttctggtgctgccttttcccaagagagaaaatccgagaggctggaggaggagcagggtttgggggaggatggtgacttcagctttggatatagcacccttgagattcctctgcagttcaggtggagatgtctagtaggccatggatgtcccatggatgtctatgggtttggagtataggacggaggtctgtgcctggagatgtgagcatagacgtatctacacatcagcagggttgatgagattgcacagtcatatcctcagatgagaagggaaagcagaatgttggagaagaatatggaaagaatgcaagggaagtgctggtggtggtaggaggaagaccaagagaggtgccgtctggaggagggatcggagtgtccagtgattcagagagcatgagggaggaggggctgtggcacacctgcctgactttgaaatgaagaatggtattctaggtggggctgtttttttagggacctggtgcaggtggggatgatggacatagggaattctactctctgggagacagcatgtggctgggatcagatagttacaaagttatatgacaactattttatttcatttctctaatactggcaacatctgaacattatctacagaggaaggaaaacctttatggaggggtaggttgaaaatatgtctgggccaggcatggtggctcacacctgtaatcccagcactttgggaggctatggtgggaggattgcttgagcccaggagtttgagaccagcctgtgcaacatggcaaaaccccatctctacaaaaaatacaaaaaaatagctgggcttgtggtgcattcctgtagtcctagctacttggaaggctgaggtgggaggcatcacctgagcccaagaggtcggggctgcagtgagctgtgattatgcccctgcactctagactgagtgacagaatgagactctgcctcaaaaaagaaaaaagaagaaagagagagagagagagagagagaaggaaagaaagaaagaaaaagagaaagaaagaaagaaagagagaaagagagaaaaagaaagagaaaagagaagaaaagacaagacaagaaaagaaaagagaaaatacacctgggatggaggccctgatggaagaaggtttctgaggaaatgggtgtgggtcccagagtgtcagtatagggcctgttggagagcgagtatgataaggtcagccctgccactcagaagaaattctggttatactcacagtaatcacacttcctggttgacccagggcagtcctaccgtgcaaccattaccccaacaaaattattgcttacaccccttttcacttttgaaagtttaggtgataaagtatctggtctcctcagagtatacatctaaggagattcgtaggtggagttagacagctgagggagctcctacctgaaggactctgtgcagcaggaggcaggattctctgtatgatgttccaagaggcttgaggagataggagcagctgtgaggggagggaagacctgaaatgagaaaaagagcaaattgttgatctacagtaagggctgagcagagcagatccttgagaacatcaatttgctgggcaccaagtagttcataggccgcgtttccctgtctttagcagcctgcgtgtctgctgggagaatagatgtataaactgatctgaggttggagctgcacagggcagatgtggccagagggtaaggaagtggctggagtggtggatcatgagaatagcacacccgtagtaagctctgcacatggagatacttattgcagcactgtttatgggagcagaggattcaaaatagtaaatgtccaacaagggggtgtcatccggtaaattatggcaaattaacgtgatagaatattatgtggccatcaagattttgtttaggtagaattgtaatataaggagagaaagcctagattgtaatagtaagtgaaataagaaagttccagaatgatgtatattaaaccatcccgcctataatgcatagagatttttttatttatttttaaaaggttgggcacaaatattccataataatacctgtgggagctgatagatgatatatttttgtatttttaaatttttcttcaataagttcattatattacttttataataaggaaaaaacatctctctaggaaaaattatttaaggaaaaaataaacatgaaaaagggtgtctgtaatcacagtttaatttggccttctcagaaaaccattgtagggagtttttattctctagtttccccagggtggggaagatgagcagcatggccctagtgtttgagaacgtgggttttgatatcagacatgcctgggtttgaatcccagctccgctaagctgagctatagtattgccatggtgtgatctcaagcaggctatccttatttatgaagtggaataatacacatccccagtctcttctttataattccgaaacccaaaaagccctgaaaacccagagtcgtttcttaaagtgacagcaaattcatttggctataaaacctaacctgaattgagctagtctttatttcacttcagtgaatataattttgcagtggaaatattaacatgtttaatcattggatgttcgcccagatctcacggagggtatgagataatacgcagggttcaggtaagttgtgaactacaggatctgcgtcatagggttattgtagaatgaagtcaggttgtgattgcagggttcttagcacagttcttgccagagagtaagtactgacaaagtgagttatcatcactattgatgctataattattcgtgagccttcactaagtatggagtgcatgatttctgcttgcccctcttccctccatttccttctttattgggatgtttgagaagattaatgtatcacatataatgagaatttaggatggcattgtttaggacacaggactgcaaaaaagggaagttccagcacagatattttcctgcctcttttcttattccagatttctcactttctggggaattagctgtaggatataatgcacattaacaggacacccaaatacttgaagagctaatggagaagtaacccttactcccttggtgactgctttcgtctcaaagcacatgtgttcattataaaatgttaatgtatttacaccatagttgctgtacatttaaaggtttactgtgctttaagctaaaactagctggatatcaaatgtgcttttaattgaaaagtggttatttactgctttccataggaatccacaataagattttttcattgcaaaccctcctagtatctttaaaatgcaattcagttcacaaacattgatttgcattcaacgtttttcagaagcctaaatagagtgcaaaatgaggtccacctgcaagactacagttattacttcctcctttttcttggaagcatccatgcattgtaaattctgtttgtgtggcatcagccaactacattattaacatcaccaggaggaattaaaatgtatcatgttataggatcaaaagtttcacattctgaatcaatgccagtataaaaagaatgtccagacccttccgtctgactacagtcggcagggatccccgccccagcagccatgtgactcacttcgcattgtggtgagggtcactcatggctcatacgggcctgcggcccgataggcctccccatggggcagcatttagccttttcaaaatcagcttaggatcacagaccctcaggtcttgaaggaactttttggagtttcctctattaaacccagttttaatctggtcatttagactgcatgtctagaaacactacagcatttttagtagtaaactgaaataaatcttcaaataaacgactttaacattttgcacattggtccaggggcagaagagctcgtctgttgccttttaggagggctctaatctttttctgtttcagctcatcgggtcctcattgtcccctactgtatgtgcccagcacaatgtcagggcacatttctacacaatgttggattccagctgatcctccgtgttaatccattcgttagtgccaaattgcagggttccttgccaagcccgtcagcacaatcagagcttctgaagtcaggtacctcaattacattagttcagttgtctcattagcccccatgcatcatccaaactcatcatcaaacgtgttttccttcattattgttgtcattttcttaataccagtcttgttgaaaaaggattgttttagatacaattttgcctaacttgtttcatctccagaatacaatcctaaatagagccaagatttgccggagagaggagatagcggttgaggctggtgaagcttccctccggttagaattagagagaagtggatgcagaacttgggccagtaatggacccatccatctccctagtcaagaggacattcttgcagccagaggtggttccgctttggaaatgatctactttgtgtaaaccggtgcatcacagatactgttagagtatctcacagatactgatacagcatggtctgcattttgtagatatgagggctctcccttaggtttacagccggataaaggagctccacttacctggtgatcacaggcatgtggtccaccctgctgggctctaggactttgaacatagagaaggctatcttttaggaagactgcaggatcaggagtcgggaagcagggattctgcttgtagttttacatctagcagtaacaacttttagcccatccctcacctactctgtgccctggatttcttggctttaacatagcaaaagcctctctttatagttttcctaactccatctctccccaaaaagctggagatgtaagttttaaattccagtccttggcacatagtaggtgctcaatctctcatccttctgtccctcaagatagccgatggctcagccatatatgttccagacaggtctctctgtgtcttttgggaaagccttgttgctctcagactggcctctaggcctcatgttttcccacttgctcttgccttcagtgttaggtcatgggcttgcctcattacctgtttcaaggaggcctctatttgatccctggagcatccctgggagtctgaggcctttaaggactcctgtggtctcaaggaaaaagaactataagtagttctcaggctcccttgatcaattcaaagtcatcttcagctcttccagtgcagcccctccagagctgttgaacgccacctttttcctctttcgggaaacccacaaaaccttgcatggggttgtgctctgtatggttttccagcatcaaattcactcatcaaaagatgcatcaggagggtggagggggtggcgcctggggtgagggtcaaacaaggaaggccagaatgcctgtttgcattcgcaacgggaacattggaagttcgggtggaaaaacaatcccaccggcctttatttaccacagtgggtagcctcgggaggagggggagctgggtgggaaaggaagacaatagaatgctgtgttgccttgctgtaaaagcttgctctaagcaaataaagtggcaagctcagtcagagccctgctcaattagccccagtgcttaaaggagggtctccctgcagctgttggcggcgggcgggctggagcaaaggcaggcgcactctggggcactcgggaggcgaaccggcaggaatcttgcatgggagctgacccgggagggagaccaaaggaccctccaacctgatcccagtccctgcttcttgaacagagggctacagaaggggttggtggggccactctggggagaggcagtgtggggagaccacggaggaggtgaccagaaaagtgagtgcaaacgtttcatgccgagagtgaccgacatatggaacgtgttatccggaagggttacaggctggaggagctcctagagtttttcagggggtggggtgggggagggggaagccttcagaaactcaaggaggtttctaaacaaaaggaatttgagaagggttggagaaataatgagaaatgcaaacttgacccagagtttctccttccatctcacggcttctcccctgcctgtcctcacatgtttccagttcatggggacccaggaaggccactggagccctgtgcctgactccacgtgcacctcactgggggtgtgggtggcggggtagggaggacccgcagagctggctcgctgctctgtgctgaaagggacccagagagcgagagccctgcctggctttagacccctgtggactagcaggctgctagcgccgggatggtctctggatgatttactccggttcctgcccttgctggaggagcaatttgatgccggactgggagtgaaaaacagacttgcccagggactcacagtggccaaaggggaggctgggagtagacgcaggctcttgatcccttctgctttttggcatctccccttctcagcatcccttgcccagcacaccacgcacacatgtacatacacacgcagacacacacaccaaacagatgcacacagaataacaacaccacacctacatgcacacatacacagacatacatgcagcacagacacacacagaataacaccacacatacatgcacaccacagatacacagaataccacacacacatgcacacacacagacacattcacagaatacatatatgcacacacacacatacatacatgcaccacacagaataacaccacacacacatacagacacacagacatacacatacatgcacacatacagatatactcacagaatacacacatgcacacacatgtatagacatacaccacacaagcagaataacaccacacgcacacaccacacacacacacagaataacaccacacacatatgcacaccatacagagacacactcacacctagacatatacaccacactcatacagagacacatgcacacacatgcatactacacacatgcacatgcacacacaaaaacatacacagacatcacacatgcatacatatacacaacacacagacacacccacgggcttacacagacaccacacacacgtagatatgcacacagcacacacacacacagagaaacagacacactcgcaggcatacatgcatgcatacacacactacacacagacacactcgcaggcatacatgcatgcatacacacactacacacagacacatctgcagtcatacacaggcactacatacatagagacatacaatgcacactcacacacagacacacatagacacatgttgtgtgctcatgcgtacacgagcgcgcacacacacacactgcctgacttgtttcccacaagacgggtactggcctgtcgcttgtagccctcctgtcccagcctgtgttggccagggtgccaggcactgccacccctcttgggacaaggtacaggtggccagtgtgatcacggcctgttctgagagcctcttcctgagccaggaagcgctgtgtgatactgagtgcccgtgccttcgtctttcccatggtgctgggtcttggccacctgctgcattgatagcacccgcatgttcacttccctggcagtagaaagaaatgtaggttagtgcagggaggtcactggctttggagatggtggcagggtggagcagtctctaatgtgaatggaagtgcacatgccctctggacctgcagcaatgctggcttcagaggggccttctctggtacctttccaattttccccaaccaggtagaagagccactgcccagtgtcttgggctcaactgaaaccccctgtacaagaaagagacccccctttccactgtgtctctctcctcctccccactcactgtctctctgtctccctcactccgtgtctctctcctctcttctgtgtctctctgcctctccctctctctgtttctctctgtatcgctcttttttttcaatctctgtctctatctctcctcctttctctttatctctctgtctctccctctgtctttctctctgtgtgtgtttttttttctgtctttctgcatctctgtttctgtctccttctctcggttctgtatctttgtccccctctctccctatctctgtttctctgtctcagtccctttctgtctttatgtctctgtttatctctctcagtctctgtatctctgtctctttctctttttatcctttctctgtctctttgttctctatccctccatctctctctccctctccctcagtctctctctgtctcttcctttctctgtctcagcctctctctgtctctgtatctctatctctttctctctctcccagtctctctctttctatctctctctctctctctctctctggcactcactcacccacttacttgaagtctccatgagcagtgggtggcttcacctttctgtttcgccactttgcagtccgacccgtggggcttgcagaccctcttctggcgcacaccttcaggagaccaacggtgccagggcactcccgtgttcttcagggttccagccccgagtagttggtaaacatcagtaatcgtcctagagatccactgtagattcctcatccaggtactgaatgaggcccttctgagcaaatttaatggaatgaccttggtgacattacaagatgacggctcatctcctgtagtctattattgttgggcatttaggttgatactatgtcttcactattgtgagtagtgctgcagtgaacatatatgtgcatatgtgtctttatgaaagaatgatttatagtcctttgggtatatacccagtaatgggattgctgggttgagtggtatttctatttttaggtctttgaggaattgcttaggtctttgaggaatcgcatttgcgttttcaaacatcatggagaacacaggtcttgaggatgtgcaggaacatggagaccaaggaatgaaaaagccatcaggttggaagtgaagtcacccagagtgattgcaggtttatggacagaaaggaaaatgctctgattcctggtgtccatgaggaaggtggatgccaatagatggtgctacacagacacttgtgaaaggaagaagggaaagagtctgcctccatgggctgagtagggctgctggggcctcaggcttcacacatagtgtcagacatgctgtgttggtcaggttcatctgggacccctgtgcctcccccaggaaaccctattggtaggtgtgggcaggcttccctccatgtgtccttaaatacactgactgctctgtgtgtgactgtggatgagcagcgtagggctggtcatctggagggctgggcttggttccagctcttgtactgatggattgattggttttgggcaagtcacttccccccataacctttccattgtaccacaatcctaatagagttggatgccagtttctggcaccctgggcttcacagggacacagagaaacagagaggaccagaaaattaggctgataataatcatttcttctctatttggtctggagaagaaatgacttagggttggcacaggttaatatgtcaactagaaagaacctttgaaaatctttagttcaagaattctaaacctaaaatctgtgggacactaggatgatttttatggactctttgaatcaccctgaaattttttaaaatattttttctttatgggcacatgtgtggttttttagcagaaggattccttagcattgattacattgtaaaagggccaacaatcataaaacaattaattccccagcatcatgttatgtgattcataactgaggggtctccgcagatggcatttggaaacgtgcgggtcctttttttcagttgtcagcatgactggggtcacgatggatatttagtaggtgggggcctgcgttgcatgcgtgggggattaaacttaacaataaaaaactgtcctgtttgaaatcccagtagcacccctctggagaaacactgctcaggaactgagcccccaagatgcacagtgattctcccgtggccacaaagctcattagtggcagagttggtttttattaacagatcaaaacaggggatgtgccaagaacctacattattttgtttctcatcatcagctgcaaatgtgctgcaatctgtgaaaacaaaagaatcagaaatctgtcccaccttcaacgctccagccatttttaaaaatgagtctttgggttcttggttgttttttaccatcaaatgagaaatgaggaaaggaatattttacatttggagaaactaaaccataaacaattgatcccactagccccatatcacctagatgtgttcttcagctactgtgaattggtgacgcaagccttagactggaaatttcccctttatgtttcagaggatctgtcttgaatgtctcttactctacaaagaaagaaacattaatataccagcatgctgcttgctacctagtttactgcaatgaagtggcaggtgccttagactttggagtgaaattgagagatattccgcagtattagctaagagttaggcctgtgggatcacagagacaggggtttgttccctggctctgtctctcaattgatgtgtcttcttaagaaaaagtacttaatccttctgagcctcagtttccttggctgaaaagtggggataataatcgtatccacaaagattaattgagatcatccatgtgaagtgttccgcacagtctagcatatggggaggctcaataaatgtgagctgttattacaagcgattattatgacttgtgtctgtgattaaagacagcctgaggttcagagtgtttgtgccaaaattgctttcagaggacaagctctgggttggtttttcagtcttgcccagctgcccagagtcactagagtgtttggggctggagtcacagtgtttattaagtgcctaccacacactccagttttgatggaagattggttttcttcctgtgtgcacatccccgcatcactacatgagtgggtgagaagagactcagaaaagcaccagacacatgtttcttctgcctcagttccctagtctgtaaaagtactcaagagcatgatggcggccacagggaacagaggggagagacacctggaggagcaaaagacagtcccagctgtcagtcttcagacaccatggttttctggagaagggatccagaccagaccgccaatcaatatagccgcagaccgccagtcaatatacctcatccttgtgaaaggcggttgtctgtgtggcagcagggagacaggaggggccatatttgacacaagcctgctggccaagtctaaaggagtcagcctgccatctgaccacactttctgcagccaagtcctctgggggccaaatcagtatggtttgagtttatccccgacttgctgctatggttgatgcacacaaaaaaggtttggccattgccaagccagctgtggtcttggtttggaagaggcaaccaaagtaagaattgaagggaaggcatgctttgggatttcccttagccttcaaccctctagaggaagccaacttctttattctgccattttgagactcatgcttgtctgggtggataagataaatactttcatcattatcatcattattattaattataattattgaaaatcaattactcagcagcccctaccttgtaatggtatcacttacagttgtatagcattttgcagacaacgtatacactagaccataaactcagctcgaacataagcataaatttaaaacaaaaacattgacttgggcagctattatctgaggtcaaagttattctttaattccagctgtaccttcagctgcctatgtcgctgaagcaacttccccttgtgtgttttcacacttagagattggagtaatccagacacagaaaatgataccaaaattgaaatgtttctgaacgttaaatgattcaataatttgaaaatagtttcaaatttttcaaaattcaaaaatttgaaaatagttcgactagagcccatcctcacctcaggtagttacgtggtgtgtgtgtgtgtgtgtgtgtgtgtgtgtgtgtgtgtgtaggcacacaaacccatgcatgcacacatgtatgcacagtggtggatgagtgtgagttaccaaaacaaataccactaaatgcaggataacaccatgatggctagtagctggattgactcagattttctctgcagatccttcctagtccaccctccttccatgacatgggagtttgtgatatgcaaaggataaagtaaacctgagtttcctggggcttgtgtcggcataccaagctcctttaagatctctctttgccaagatggggctaagccagaggcttccttggcctggggaataactgtcctaccttcctccctggagcggctggcgcacactgttttgacgtgtgccatgccttccatcttttacggcctcggggccagaggagggtatgctttgaacaatgtgaaattcctctcctaacagctgtgcaaaggaaactcacagctttctatctccatctcggtccacttagctttcttttggggtataggtttcttgtctgttcaggggtccctgttcctcactttctggccttcagaaggaccagtttcagtacttcctttcttagggaggcaagtcagtcttctgaggttctctgttttattttattttatcctagagtatgccttcactgttgaccgtgtcttgcctcatttctgctaagaagccagcatcttctgaaaattgagtgtcttgtcctgtttcagatcttgggccgaggattgaccatgtatgttagtcagtttcagagccctgagttttaagtgctaagttttaaggagaggaacaacagtagaattagatatcttaaagaacaggataggaatccacttccctgccccttctctcacctggtttgacactacatagatacagacctgtcctgagttcagggattgtctaatgaggcctcaccaaataccaacagagagactagctgtttctcctctagctcactgactgcttctagttttccgtgactttagagcatgtgcgtcagcaggttttggggaccatctctggcctgctttctgatcttggctctgtccctcaatttatgtcttttttgtgctccataagacagggagtttcatggttgccactacccgagaggtgtgctaaagtgaatgaggtggaggacacagagctagcagggaagaaggtgccgtggaaacccacacagctttattcatgtgagctcatttctcatttactccttgtagcaatgccaagtcaccccctcccttcatccctttccaagcatttccctcccttgttggttttggatccattgtaaccccagtgagttaacctacatgttttagtttgaatctaatcatgctatttccaggttgccaacccccacgggtgcttcagcctaattggtctgtcctcactggtgtctgcagcaccttccctttgagcgtcatctgtgcattttaattaatctgccactttttcctccctcctgcttccggatcattaataaagagattaaccaaaaccagaccaatccccagcggaggctccaccaggatctctctctcccagctctcagctttgtcatgggtcagaacatcatcatgtggccccattgactaattctgaggcctcaagacctatgttcagagcaaagccagtgtgaattaatttttcaattaagatttggggaacaagcatagccagtcttttatatatggccttagtttaatatttccaccacattcctttcccttttgtaattctactttaaatctatcactttcccacctctctccctttccagtccctgaacaagacattagggttatctggcaggatgtctcctgagcctgtagcattagtcagagatcatgtcatagccgaagaaattctccaaagaatcaggcccttctggacacagctggccagcgtcctctttgaccccagccagagaatgcatttgcctccagagaatatggctacaccagaaataatgaaatctcttgaatttgcatattaaaaatttggcaaattaaaatttgctttcacctctgctctcatgactcctctctgaggaaagctaaaacagagcctttctgctttctttgtaagtcacccagatttccagcgctggtctcaaacccaggatgtgagatggtgcactccgtgttctgtccactacaacacagggcctctgcaggcacaggtgacctgcagatgggctgcactggagagcatcaggcccaggagggactagggtgagggaattggattgtgtttacttttgtgggggatgctttcatagggaatacagggaggaggacacaggattgatggcctcaaatctctgcaaactctgcttagagctgaggcttcttggggaaggacacagcctgctgacagcagccgtcctggctccaggcttggagcttgggcttcccctggaagtaaactctctagaatgtttgagagcaaggacagacggagaggcagccgagtgtcatcattaataacatgagctctagagccagaaggtgcaaatcctgactctgttgcttgttagctggttgtggaacccctctttgccttggtttccccattcaaaatgggaaatataatagtacgtacttcaggatggttgttactaggatttaagatttatgtaagccagtacctggcactttgcatttttattcttggtgatctcacccctaagcagtctaaaaatttacttcacacatgtgcagcaagaggcgtctttaggtacacggtcaggaggtgctcctgtgtagggaagctggtcctggagaggtgaattggaacctgaaggctgcagcccagtctctgcacgtgacagggcagacgcaggttgctgggggctcagattctgtgccactgtctacctcagcttccttttccatgtcagctgcttcttgaatacccccaaaccctctgtggatgctgtaaactgagccaaaaaggaggaatcagaaattgctctcttttgagtattttgagggatggtgaaaccttgtaggaatgttgctccagggactgagaaaacaccatatgtgggaaagcgtttctaagtgaggaaaatcgcacatgcacgtcggtcaggatcttggaaaactctgagcaggggaaatgggcactggggagagtgtagaaagggggttcactaaggtctttaacaggtctttttctaattatagccaagaagagcctggggaccatggccgacttcagagccagggcctgtattttctttgggaaaagaaggcagagttgattggcttccaaaaccaggcttggcaaaagcccatgcaggctctccagccagtaagcagtgcctggggtaggtctcgatcgtagcaggcgctcattcagtaaatagacgtcgaaagtccgcagcgtgtccagccctatgctgggagtcaaggatgtgtggatggaaaggacatggtccctgtcgtcaaagagaaggcacaccaacaataatagcacaatatgacaattgcttaaatgaagctctctgcagatgacagggacccctagagaaagagcgtagacattcccctaaatagcagaactcgctgctgtgccttgcctggatggagctggcactgccttcctgtttccattccccactccagggaactggcccactgggggactcccaccccggacatggagtgagaaatgaaggtattggtggccaaaaagtgaccagagatcagaatgcatctattcttgcccccagcaaattcagaaacaccacctctcccttgcccgtgtttgctttcgtcatttgtctgttctctgggcttcgagttcataatattcctatcttctcccacaaccagtgtaactggtctgagatccaagcagtcgggcaccatggcatctgggggaaagccttgtggggaccgtgttggtaaagattgctgtgagccagatgtagaggagggagctctccagactctgggtccctcgcccgtgtgcgtcaagggcaggtgtgcaccatctcccgtggccacaggccattcagcttcatgttctctaacatttctagtgtgctcactctgtacctgatgctatgctaggtgcttccatgtggatttcattgtttacccctcacgataactttcatggtgcacaagccatgtgatttttgttgcgcctacgaaccaaatgcccccgtcactctctgccattgtttagcagtctgaccttagacagctcacttcaccacaactgcaggcctccgttcccttatatgtataaatgaggaggttgtctagggtcctttccagctgcaaacctctgtgttttggggagttctgttagcaacattcttggacttgctctctacgagaaaagctagaagtcgattattaaaatggagtccacaggaagcctgcagggctttaagacagccttctaaagagtttagaaacactcaccagcctgcagatgacttactcccactacttattacaaagtatcatggcaagtaagaaagaaatggtcagccactgggagagcaggctgaggttgcaaggaagtggcaggtgctatcgttgggatcttgggggagcattgtagaaaggtcagctttggaaccaggcactgaaaaacgggtgaaatttcaacaggagaggcagtgcacatgggttgtgatttgagcagcagtgagtagtgaatggggttggagcaagaggcctgtgagcccagtcatcacatggaaaagggaggtgagggccagagtgcagagaacccctctcactttcctgtgggtctctcctcttctttttttttttttttttttttgaattggagtcttgctctgtcgcccaggctggagtgcagtggcgtgatgttggctcactgtaagctccgcctcccgggttcacaccattctcctgcctcagcctcccgagtagctgggactacaggctgccaccgtgccctgctaatttttgtatttttttttagacggggtttcaccttgttagccaggatggtctcgatctcctgacctcgtgatccacccacctcggcctcccaaagtgctgggattacaggcataagccaccgtacccggcctttctcctcttctttaaactgcatttaggttgtcctcatctataagatgaaggaataagattagatggcctctttatatggcttatctacttctaataaactttggttccatattccagccaccacccataaagactcttactttttcctcctcaatccatcagcatcagccagcattctccctctcagttcatcctggtcattacctcactaatctgtaccttgactcttattctacctgggacctcccctcactaaccatacttgatttatttccttaagagcggatatcctgtgtattcttagcttacagtggctgagcttttgggaggtttaacaaattgtttttcaatagactctcagtcctaaatgattttattgcagctgtattattctttttgaggggagtagagaatgtaatcctagttgtatgccatattttttgtattgttcaagttgcattttgttgggcctgtaatattgaagaaaatgtcacttgtatgcagaataggagggagattccttatgctgtagaggaaccattttcctaggatagtacaattccttagagtcatctctaagggtgagcagcaaagctagaatccctctcttggctttgaccttgaaggagtcagttttcccaaagactagttcccatggaaagaagatggtctccttatcacagcagcaagaggaggcagtatgagcagagtgcctcactgttgttgacctctagggacaagtgagccggcagtatttcagacaagctgggaaagaggctgtctgtgagtcctgggagcgagtgagcactggcaggctcagacataggtgctggttagcaggactgcttttctgtttcttgtgtcggctttgtttatttcctctctattttccccctggacttagtaaagtctttccgaaaataccaaaggtgaaccaggggaagagtttttattttccatgtttggacagaactttaaagaggaaatgatgtacccccctgggagccagtgaggtggcagcgatggtgattaaggagtgaatatctcaaggaggtggacgaattcggggatcactagctcagctgcccctctccacctggagcatctccttccagtgctaccctcagaacatctgggctttgctctagtgagggagagactagcaatgaaggtgtcttgagatcagcactgtaattccaccaggacgccaccggagtccggtgttaagcttctactatggcaacagaatgagagcgtggatgggttgaaatgccatttcaacaaggaaatagtagaattagaagaagcataagcactaaaaggaacattttgtagcagaaatgttaaaaatctaaaggaggcaagtgaatcaacaatgactcttctattctgggcaagtccagcccatttatgtaaggtggttattctgcatctctgtcttctgcaagtagtgctgtggcagagctgcgttttgtggagagcgtccccggggatggagcagatcagttggtgatgcgtatgtatcagaaagctcggcagagcaccctggaacgtaggccctctcgcggagtgggtagtggccctacatgttcatttccaagggcaggagaatagaccgttccagctgcggcctggccagggatgaccccacatctgacactgcaatatgggggcaactgaaccagtcctcagcctcagtgtgttccaggggctgcagctggggagcagtcgaactctttcttgagacaattacaaggccaccgctgctgctgctgaagggaagttactccatgtttacaattctcaggtttgaagttttcatgctttgccaaggtagagtgaaccatgcgtctttgcaggctcaagggatgtttaaagaagcggtaggacatcgtccacccacaagcagagaccgcaggataaagcagacatccaatgtaaatacaacccgtgcaaaaagcagagtcggcagacctggagtgcattcgcagtatctcccgggggtgggggaaagaaatcacctcttcagaatgtccagaggggagttgccttgcttacctggggggcggtaccctctctcgtgccctcacagggctactcagcctcaggtagctggtgccagaataacacagactcagctgccagagcctgctcttaacacctgtgtttccttttcagatcttacaggtgaacaaggtgatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatgaattggcatctctccccatatataaattattactttaaattatatgatatgcatgtagcatataaatgtttatattgtttttatatattataagttgacctttatttattaaacttcagcaaccctacagtatataagcttttttctcaataaaatcagtgtgcttgccttccctcaggcctctcccatctgttaaaacttgttttgtgatccggctctcaggagtcactctgtaaaatctgtgtacaccagtattttgcattcagtattgtcaaggccatgactgttgttttagtaaacttgttaaaatca

SEQ ID NO: 31 - protein, 255 aa, Homo sapiens
MFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 32 - protein, 249 aa, Mus musculus
MTILFLTMVISYFGCMKAAPMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 33 - protein, 249 aa, Rattus norvegicus
MTILFLTMVISYFGCMKAAPMKEANVHGQGNLAYPAVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 34 - DNA, 744 bp, Homo sapiens
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 35 - DNA, 67164 bp, Homo sapiens
attattaaagcggtagtctgccggcgctgataagcaacaagttccccagcggtcttcccgccctagcctgacaaggcgaaggttttcttacctggcgacagggaaatctcccgagccgaattcagcttcgccggagccccaggtgtgacctgcgtagtgggcaagggagcggtgtgcaggctgagtttttttttttacaggggtaccctgaaactcctcactttctctgggaactttcagtgccaggacccagtaacgggcggttagaaggcagccctaggaaacacctgctacatagcagggcagttgggcaatcattggtaacctcgctcattcattagaatcacgtaagaactcaaaaggaaacgtgtctctcggagtgagggcgtttgcgtaaatctataggtttttcgacatcgatgccagttgctttgtcttctgtagtcgccaaggtggttgagagtttaagcttgcggatattgcaaagggttattagattcataagtcacaccaagtggtgggcgatccactgagcaaagccgaacttctcacatgatgacttcaaacaagacacattaccttccagcatctgttggggagacgagattttaagacacttgagtctccaggacagcaaaggcacaatggtgagtagcaataaaacctgcattataattgaaaaatcttgacatgttgcttaacaacgggcatatcacggctcttcctagcacttcacacgccaaagaacagcagctactcaggccaggggaatcgggtttttacacagtgcaactttaattggaatcatttgagatttgacacagctatgtggaactgcgtggaacaaacttggagctgggtgggggggtgtgtgttatattggttgttcaaggctgatgcttgtctctcagcagtcttgcattctattcttttccttaatgtgtatggtgtatgatcatattctatgatttatatgtgggcatgtaattgacatttgcaagggggttaatttccatctaaaaacaataatgctgttagaggttggggttagggggtggagtgggggtaagggtggggtaaagactgggagtttaggtgtagatggggggtggggttggggggagagaaataagtcagaagtgcatatcaccggtaatgggtaatcctctcgtagaagaaaaggttctcatcaacatgtgatcaactattaacaggatggctttggcaaagccatccgcacgtgacaaaccgtaaggaagtggaagaaaccgtctagagcaatatcaagtatcacttaattagagatttttaagccttttcctcctgctgtgccgggtgtgtaatccgggcgataggagtccattcagcaccttggacagagccaacggatttgtccgaggtggcggtacccccaggtagtcttcttggccccgctgtaaagccaaccctgtgtcgcccttaaaaagcgtcttttctgaggttcggctcacactgagatcggggctggagagagagtcagattttggagcggagcgtttggaaagcgagccccagtttggtcccctcattgagctcgctgaagttggcttcctagcggtgtaggctggaatagactcttggcaagctccgggttggtatactgggttaactttgggaaatgcaagtgtttatctccaggatctagccaccggggtggtgtaagccgcaaagaaggtaagcaccagggcggggaccccttgcatccccaattcttgagctattttgatactgtcttccggagaggacgcgtggtggaggggaggaggtagagggagagcatgagagggggttgtttcttggtatttgcccagtttgaattgccctaggtgagaaccctggggcaaagggagaaagaaaaaaaagaaactcagtcttcctgcggatataatgagtttagttaacttggacctgcaaatgtctgattcaaatgtaagatttatctctctttttctcctcttcacctccctcttttccgttctctttgctggtgtgtgtgtgtgtgtgtacagtagattcattactaattatgaagcttttgcaaaacattcgaattcctaaaatttgactttgtagcatttagaatcaggcggtggaggtggtgtgcggtggggagaggaggtggaggttgggaagagggaaggaggtaaagctaaacctccaacacaaaaaaatgaatcaaggtaatttcagctcttctagtgagaaggattcattctctctgtatccctccctccctctctttccccctccctccctccttcccgccccccttcttccaccccgccccctcctccagcctccatccctccctcattctatctcttcctctccgtcgccctcgctcctcgctggatgcttctttctgggttttcttttttttttcccttctgtcctccctccccgcgagtttcgggcgctggcttagagggttcccgctttctcaagggaaggggagctgccgagaccgcgctccgctccccagccgggccggatgcctcactgagcccaggtccgagtcagtcggggtaactcagggaaaggggagcctccgcctgggagtagaaggtcctttccggaccgaagagccagagagcgggccgggcgagggggcctgggcggctggaggcggtggagaagaacacttttagctccgtgcggcggctggacagagccaccaatcagctggacgcgcagaccgccctgccagggcgaggttgcgtccggaggcgccggtggagggcggccggctagtcgctgagccgccgccgccacccgggtgggcaggggactggcggtgggtggaggtgaggggcttggcgggtgagatagaagcggcgcggagccgcccagacctgtgttctacctctcccgcccccgcctgcacccccgggggacagcgaactgccggaacgcgcggctgcgttatcctcttgccactcttcagggagctcagggacttaggcgcccctgggcgggggccaccaggctctccacactcctataaccctcacccccacccccttctcaggccttttgttccggccacagagccaagcccggtggcagttttcgccccagggtttggtcgttctggactcctcccccgatcagctcttcttaattaaaaggcaagagcttggggtaggggtgaggggaacgtaggaaaaatctgtttccgaaactcaagaccactgttttaacgaacgaaagaaagaatcccaactctgcgcaggtggattcataggcgaagcgaggatattgtggaaattcagaaggaaaagataaaaaacaggcgctaggatcagatgacggtgataggctgctcggcacacaaagggagcgtagggcagggtttacggagcaagcctgcagcgaatggggcacagattgttccgagatccagtcgttttctcagtcagatctacgcgaagggaggggaggggaggggcgggcaggggagcgtggcgggaggggctgagcttgggggcggggggatttctgatcagtctgatgcaattccaagcgtgctgcaaaggaactccaaggcgcccgcatcaccatcgccacccacccttcccagatggtgctgttttaaatacggatctgcagggctgaacgcagaactgggagatttattgcaaaatcccgggaggggcgggggggggtggtgtgcggaacggggaatggaggagcagaatttaaaggtgcaacgcttgctttttccaatcaggcggcaaccggccggaattattatttttttctttctgtctgcttgtctctggattctaattcaccaagaaagaggtgtaaatattgtgacattttgaggcagcttgatggatgggaaagaaatcatctgtcactctaaattgcagagttccctctccccgcgccatcccttgctagcgaatactcgctgctgcctaatacagttgctagggcttcaaatgaatgcatcgttaagggaatattatccttttagttgacttgccaatttagttgacagttgaatcgagaaaattgtagatttcgtgtctctgggaggaaaaatgcttaacagtctaagtcttgtaaccttgaggtctttaacaacttaaataaacctcaaaagtgtcacgtcatcctctacacacacacacacacacacacacacacacactcaacttgtaagatgacatggtttcacctaaactgttgtggaaatgaatagcactttaaaaatggtgcacctgatattcactgtttatgtgtatttacaaagagctcttcagcatgaaggcaagacatttcaattgtcctgtttggaatcagtcagaagactagaaggtgatggagagaagaagggaagaaagaggaaagagagagaattttaacctagatgctattaaattaacagtaacctagcctacttttataccccttggtcttgcatattaatatttctgtatgtgagattttagcttggtctagctcccccaatggagtataccagtattgattcagatgagaatatgagcatcctgccagtagctttttcagtgtcattgatagtaagacctacaacacagcaatttttggaggatagaagagaatatatataagggctttgcaaactgggaagcaggcactccataaatgggaggtatcattatgacattctctttgcacatatcattttcatttcactgaaccagagtactagttattttaaaacataatctaatgtatatgctcaaggtagtaagtggggattttaaaagcaagtgattagttggcttataaaatattatttttcaattgtctattaatgtacattggaaagaaggcttttaaagatctaaaatcaacataaataagcttcccctttcatttgccagactctttccctatcagatttctgatctaaattcttaataagaagagaagctggtgaatttagtttctttccttttcctggcctgtcctctaggggaagctttagtaagaaacaacattccaaaatcaggcagtgagcgagagagaaggcaagggactggatgaccacaaaatagataatcagccaagaaacagaaatgagggaaaaccagcattaaagcatgacttacaaagggtttttattttgtaattctgtaattttgggaccaggctcaaacttgctcaagtaacattcactcgatcatattgcttacaatctgtcagttaaaatgatgtctaactgagcatattttttattaaatatacttctctcaaaggccagtaaagctactctttggttttaattagacaaactagtctaaccacttaaataactctaatgaatatgaactgatatcatcagatttaaaagctctgctgaaaactaaatttattctgaaaagcactgacttgccagaaaaatatctatttttgcagctttcttttcactctatggataatttaatgagttgcttattttaattttacaactgctacctcagaagtatctcaaattatctttctttggctggtgtctttctctgctgatctgctactgctgtgtgtgtgtgtgtgtgtgtgtgtgtgtctgtgtgtgtgtgtgtatgggcgtgtgtgtctctgtgtgtgttttctagtgggaatttaacaagcagtgagtctcttaaatttacatgccataatctatgtcaagaacattgcgtactacttagcaataaaaataaacattagcatctagtgaaagcttaccatcattgagtgctatggaaatagaggtcttaaagaaagattaaatttttcaacaaaaaaatttttcccctttttggcttaaaggtgattataatttcaaaaatatgacatctttcctcttttactttggaatgtagagctgctgctttaacaagtgtcttttgagaaagatacacgtgtttcataaagattaatacccttaaaacactatggtgcagagagggaaggatgaattctttaaccctgcctctaatctcatttggcaatttttggagtattcattctgactttttaaaaattcaggtggattttttttctgctttctttccaacattataaaacaatcctataagagatttttctgctatagtgcagactttatttgtatttcctagtaataacactttagattcatatagtactttgacagctctctataggtttcatttgatttccttatcagtcatgtaaggtaggaatcaccaatcaccttttacagatgaggaaagtaaggtgcagaattatctaacactacactgccagtaagatgtaaagactaactcagagtctttcttcaaattttaaggaaatttgtgtttgttcctctctagaccatgctgccttaaactccactagggcatcagagggagctgtaggcattattttctcctattttgatttatttaattaaattaaaaacattttttacaaatagttttcaaaatttcaggcctaatggaaagttttaactagtcttcctaagacagtatttccctctcccacagttagacactcaaagaaagcaggactcttcctctagttgacataccatctaagtcatagttgctaattccccaaaaaacaaatacaaagaataagaagaaccaaagccaagatgtacattacctttacttgtgaatcatagaatctgggttctgggaaggttctcagaggtcacttagcccagctcatatctgatacatgaattcatgctggcttgttttgtttcaatcctctggaatttcatcccagtcttggacactgggtacttgtttatcttggtagatgttctgatacttatgtggatcatgagcttggctgatcatttcccattttgcccaaaaggtatactttctatagagactctagcattcataattttattttgtaagtattaattggcaacacataatttgccattatgtgccaggaactgtattacaatctggagattcaagaacaagcaagaccaaatggtccctatcttggtggatcttgtggcctagagacaaagactggtctctttatttgctccaatccttagaaggggacagacctctggactcataagattccttttttcaagagaacacagtgaataagattaaaagcctgagtttaggctttacttccaaccgcttgtcagtacccaaaaaagtcaatcagtcactcttgatctctgcttcctcacacataaaacaaaagagatgttctcagaataaactctaaattccacttccgttccaagtttgagtgatataaaagatatagtctataactatttcttgcagtgtaggggaaattaaggcctaagttaccctaacctttggtgatttacaattctgggtgggtaccgcaaatttttaacttgttaagaagtatatcatgaaaaaaatcaaagtaacatatttgaacccagaatagaaggaatctaggtgttgaacctgttcatttatggaatgatggctaggaaagttttaatttagagaatgatctcaaatttctgcggattatttttaaaagcaggtggtgcgatggaagacctgctatcaaatttactgcttattttctttgtgcaagcagaagttataatttttcaggtcatttccttttaaaatcaaaaatattacatcctgaaattgcctgggtctcatgaataatgcattatatacacatgataatagataattagatggacaagccaaaagaaacatgaaaggaagcaggtagcccaaggattgcagaaggtgtgtgggcattttgacatccaggaaatgctatagatctgtccttaactaactcagcctggtggagataattaagaaaaaaaatgtgggtgtagaaagactgcaagccattccctgggattggctagattgctgcagtagttcaaaaacaattggcacagccacccacactagacatgattgccctttgatgaggcagctattgacttttataaagatgtcatatttaaaataacttctgatgcactaggcataacagacatcattcttgaattctattttagacaaatggcaaaatgtattacaaagtattaatttaaaaataaaaaatctttaaagtctagtgtctaaaaaccagcagtttagtaacatgcaacctctggatttaagaattcagcctgaagctgggagaaagctgtagcttgtataggacattttgatccactctgggcatttcccagaccactacaggaagtaaaatgtactttgtcaaagtttttaacctttgagtgaatgttaaatccactccaaaatcttcgcaacctgggaaaggtgatccaacaattttcctaaatagcggcagaaaatgctctgagatctttgttcccagagtgaatgttataatgttatgctatctagaaatttccttgtagcaccatgctcatcagtaccaaaaggagttagaattgattcctcccgcttcaaggaaatatatcaaccacctcctgtctctaagtaacaaggttactgtggggaaaaaatacacaaattaggtgattgcagaaaggtgtcacaaacatccaaagcctttgggatagggcattgcagtgtgagtgaatagagaaaagaaagagaatgtgggaaaaaattgagaaataaaaagggaagtcacagtggagttctaattatacaggggctcttgaattgactgttctctaccttccatgctcattgttgttctggctactttagtaggaaacaatgatttcttctgctttcaccttcctcctccgctaaggacttcttacttgccaataacttccataatcaatgtttaagaattgctctgatgcccagtgtggtggctaacgcctgtaatcccaacactttgggaggccgaggtgagtggatcacttgaggtcaggagttccagaccagcctggccaacatggcaaaaccctgtctctactaaaaatacaaaaaattagtggttcatgcctgtaatctcagctacttgggaggctgaggtaggagactaacttgaaccatggagacagaggctgtagtaagccgagatcatgccattgcactccagcctggcaaaaaaaaaaaaaaaaaaaagaaagaaagaaagaaagaattgctcactgtaatgactttcatgccatggactcaactctcttggcagtctggtaaagcttatgtaaacccttctcataaaaatgtctaaatggggccaggcgcgacggctcacacctgtaatctcagcactttgggaggccaaggcgggtggatcacttgaggtcaggagttccagaccagcctggccaacatggcaaaaccctgtctttaccaaaaaaaaaaaattagctgggcgtggtggcatgtacctttaatcccagctacttggaaggctgaggcacgagaatcacttgaatctgggaggtggaggttgcagtgagccgagattagccactgcactccagcctggatgacacagtgagactttgtctcaaaaaaaaaaaaaaattctaagtgaatgaaataaatgtataagattacaaaggaagccagtggcattgatgtacagttataaaaacatttaaaataatatattgtgtgatatagtaatatatatgctttttaatacattaaataagatctaacagcaaggtaaatattataattttgaaataatgataagtatcaatgtattttgaaatatctataaaactgacgtgatatgaaggtgtctgtgatgtatactggtgagaaagcatgcaagtactactgtgtaacatttcccacacatatttaacaacagaacacttgagaagcacttattaacacagcatagattcagaaatattaatttagtaaatgtcaacattagccattgttgtttccttcctggcaaaaggaaatcagcattgggagaaaacttttaaaattcacatttgccattagacaagctgtcaagtggggaaaggaccaaatactgagaaggccagggtatggtaagcatgtttctattgactgagcttgctattactctaacgtttatctttagcatcaccagcacaaccccattaccctagcaatccatcactccattgaaaaagataaaaagttcagattctggtcattaactcagcattgcttaagatacctgttctgacctcactaacccaagagattactgaaactcttcctgtttgtcattactacaccatgggaaattataatgatgtgggatgacatttactctgcattcatccagtgctgttatttgttttgtatttggcatatattacttaactcttaaagtaactctcagagatagattaagaaaactagagctcagagaatttaagtaacttgcccaaactaacacagaaaatctgaagtggagaagctaaacttcaaacccaaagttttctggttccaaagtccattatgaagttgtgcctccccatcttatagctaccacccagatttaatctgggtctcccattatcagatggtttacatacacattttcttacaagatcttgaccacaactctttgagatggccatgagtctcacaattcatttccaggagtgctactttagaatcattttgatctttgctaaccgatgagagattttcaaatagctaattgtcacctaccctttttgaagcccagttttcataatcataaaatggaaacagtattacaatgttttgttaggatcatatacattaataataaaatctaactttgttgagctcactatggtgagcattctgcatttccttagttcattgaatcctcacaacaatctttctaggctaagacgattatttttcttttaaagataaggaaactgaggcatcagtaattaattaactatcttaaattagcagagccaataagtggcaaagctgggttcaaacctaggtctgtctaatgtcaaagccctttttttaatcactaatctgcaaatcactattcaatcttagcttttattattataattatcatcacacttaaaacactatcaagatacagaatgatccagacataagtatatagtcactgaagagattagaatctgaaacttttcacctgcatgttcttccttccactttagtttattaacccaatggatgatgtctgactcctttcttaacttgtttagggcagttccaagttagttgacttctgagagttattgagtaagaaatgttataaattgtttggattaggatttagtatgtttagaagctatttcataagtttgcctttgcgaactgttactggctataatactgcagatgctgtgatgaggaacaccctctccaaagacacacagtggatgacaaacctccaaagctaacatgttgtttacagatatggagaagaagaggatggacaagcacagtctaaaacgtaattacaaggcttatagtccctgttggggactagaatgtttattggctttccttgtgcaattcaatgctcttcctccaaaggatccactccaaacttggaactttcctgaaaatagcatctcatttgggagcatgccaggaattggtgtctgggtcctttgtgtctttgcaccaactcagaactctggatactagctctagaaactaagctgggatatattctgggtaagggagtagcatatctacttgggcatcttcctgatacatttatttcatccatcttcctcctagagagcacctcctagaaagatgtggttttaaatgagggattggatgcatactggtatgtcttagcacacaagtcagtggtctttgcagagctgccaaaggcatataagtaatcaaagatgcggaagtctatgaagagacttcatcccacctccactctgatttattcagggaaggaccccatgaacacataatggatttgatacgtcccagagctctgaaagcagcctagcaaaaaaggataatcttgaaggacattttgatgtatgaaaaagtccacctaaagctttgtcagagataactaagtaatatgatggctggtagactgtaagtccttaccttggctcaggaactgtatatcatttggtaaactaaacttgtcgttcaaatttagatagaaaaagtaccttacaaatgatctagttcactgattcccttcatgcattgaaatcacctaaatcatctcttctttctgagataaggtctgaatgtgttgccagctttagcaaactcagtttgtagcccactgacctcatttgattgattgggcaactgaggtgcacagtggtagatctctcaatttattcaataaacaattatatggcccttacgatatctatctgaacaatcttgggctagtgaagttgcttgcccaggttacatggccagaaactgacagttttaaattaggaccaaagttcttttgactactatctgggccttaaaataatatcatatgacaaagatatttcttctgtttcctaatagtcacatcaaaaggaaacaatggacagtttgtgcaagattttagttactttaatgttcaaaataaaattaaaaacagattattactaaaacataagcataacaacactttaatagcattctaatcagatattattaatttcaaaatggtaggacaaaactaattatactttatacttcttaaatatcctatagttactttatgactattgagacactagctaaaacttgaaacttcaagttttcattgattcctatattattacttatttcagagttacttcatttggttcttttatctgagattggacaacagctttatttgattttcagcgacaaaattcttttcactcctgatcctccaccccaagaaaacaacagctactaatatattttccctaaagtgatcaagaaataaaagaggaattctagccaggcgcggtggctcatgcctgtaatcccagctctttgggaggctgaggcgggtggatcacctgaggtcaggagttcgagaccagcctggccaacatggtgaaactccgtctctactaaaacttaaaaaatgagccaagtgtggtggcgcatgcctgtaatcccagctacttgggaggctgaggcaggagaattgcttgaacccaggaggcagaggttgcagtgagccaagattgcgccattgcactccagtctgggtgacagagtgagactctgtatcaaaaaaaaaaaaaaaaaaaaagaggaattctaaaattaattatatctattaatatccctactcttaaaacgttagaaaatgtttgctcatttaaaatttttatttttaaaaccaccttatattccaactaaatactctttggagcaatttctttgttcctcatataatatccatacatataattctgcttttgtgattaacttttattactactcttctaaaattgtgctcttataaacatcagttaattaagagtaaatctgatgttttataaattctttctagaaacagagagcaaaatcatataaataacaatatgaatttccaaaagtacaataataaaaaaaaattagaaaaaaattaatctaggaaatagtcaagaatatgtcaaacttgtacatacttttgagataaattggcatcatgtagattagcatgattcttctttatggaattcaacttatttttactcactttgctctaattagtttttgtgtgcggacaagatggaaggtaatggaaatttggcttgcaaagtagttctaacatgatctacatccacaatctggttataatgctataagaatattatgtgggaatagtagttcaaatcagtatttagtatgaacataaagggacaaacaatgcaaagctaacttaagttgtttacacttggaacttatttaaattaaaaaggccagtggatggtcatatgtttggctcattcttctcaaggccttcaggaaaacatgcctatgaaataaaagatcctcaatattaaacattttactgcatttgggggacacatgaaatctggtaataaaggaagtgttggtcttcatttttctaattcagcatggaaactatcttgaggaaaactgactatggtcttagtttgtgtctcagaaatatatttagtctgaatcatggcgtcgacatctgacttccaaaattggatatctagccgtatagtacctcacctcccacacacaccaccccccattcccaggtcatgactactgtccaagcagcaaaaaaagaagtaatttcccagagtacatacatggcagtgacaaccaaccaaacaaaaaacaattataggggctggaatttaaattaatggctgtactctcaccaattcattccccattccaccccatctctctgtcttcaacttttatgaaacattatatttgtcctattcttctgtatcagcatcagcctttcctatatccaactagacttataacttcttggtgcctctcactggctgactaaggtttcagaagtacctacttacagcaaacacttgcagcagtctctttttggttacaaagtccctggacaatttctcaaggcgatattatgaagaggaagtaaacattctcctctgctaccccatttctttttagagtgctaactttattctatatctggtttaatgtcttcttaggccaattggactgattttacagacaccatagaatatctcctgagtaatgggaacaatatttctgctgatcccatgatttggtctcattgggttgttaggccataatggagacatacttgatgaatttatgaagacttgattctaggtatcatgtaggttagcataattctcttttactgaattcaacttagttttattcactttattctaactggattttgtgtgcagaccaaatgaaaagaaatggttcaatttaggtgaaaggtaaagcttcaaaagtagtgtagtatttcatagaccttacctttgagagaaattatatcagtatataataagcacctgagaatatgaaagcacaaatccaatttaaatgtgaaaggtctacaacttgggattttaaatggagtacagaaaagccactgtttcttaaacaattttgttgagggggaaaacagtgaaagctaaatgttctattcaagagttgtttcttttgaaaataatgcttcatttaaaagctaaggacagaagacgtagctttgttatgaaggctcatctttttattaaacaaccactactttgtctccaagttgcaaagggaagatttgtcaatctgattgaatcttccctttagtttttcccaacagctgtgtccagataattcatgactcctgtgtttcctgagccctggataatttcacacacatgtctggtttggggctccacattttcagaaaaatatagaaatcttggaggaggtccagggtagaccaagggaaatgattaatgggttgaaagttggagtttatgaagaaaggttgtgagatctgatcttttgctacgagaaaagtctgagtggtgacttaataacataaggaggttagtaagcagctgttctccatcttcactaaggttgaatgaaatgaaataagatataaattgcaacaggaacaaaaatgcattacaagtgaggacttccaagcaccagcattgctggattctagatagctccccaaaagaaggatgtgtagtctacttccctggtctgcaatgacaggcctataaatagtgagaaagatgagataatctcttaagatcccttctggacctatcttttataggtctatctatcatatttagaaaaattatttgcctcaaacaaaaattatctgatttcctccctctcaccctatccactccttctcttttgtctaccttttgtaaaacactgctaaccgaaataactggggactgattaaccgtggtgggccctcccccgcctctaagtgccactccagctttgggagcaagtttcttgtccatcactaccaccccctggccactaggggcatgtttaccatcatctttctacacaccaaacctacggcaagggaaaataaaacaaaacaaaacttcctagacttaacaaatttgcaagtgtcaccatggattaaaatacaactcttatgtcctagaatatgagcatgtaaagggctaaaatgtattttatgcatctgcctgtatcagcccatagaatagcctcctgacagatagtagatactcagcaatctttcatcaactgaatgactgtaactatgaagtgaaaggcaactaaagttgagaaagtcaggagtttcggatgtttccaaatgattctgtatgccagactaatctaaagcctaacccattcttcacaaccatgcactattaaggatttcattctcaccatgcctgtgctatctggaggtagaaagagggccagttgcacatcctgctcaagtccttggtcaaaaagaccactaaagagtgctttgtagattcatgtatcagaatcacatgaaagtaggccaaattcttagtgtgtgtttttaaaataagactttaggaagttcacttatttttttctaaattatttttgcatattcttctttttcatttttttcatgaagaatttaaaatttggctgtagaaaatctctcactccaaacatcacacagcctaaataggtgagtctcaaaaataagctaatgttcatctttcatctgattcaatgtcctgaaaccctttggtttaaatttgttaattcttctcatggcttttctcctagcaaaaccaactaataccacagctatttattactgtcagctctaacttatgcccacaatctcacatcccttttgaccacgcttatagaactattacaacaagtaaaccaaatttattcttcattattaatttttaaatgttctcagcacaaatctggtaacttggagggctacaagttgatatttctcatatgtttgggggtttagtctcaacagtttcttaatggtttctatgccgtttttcttgatccaactaaatattattcccagatgggatcagcttttgaccctcttgttctactctcctagtcttggcccttctaaaagtttcttgctgtggttcctttcttttgtctgccactaatggctatgcctggttacataactcctgtaacaggtgttgaccaatttgaacacattttggtatggtattgagctattcttatggttcataaaaagcttagtgagaacgtaacatctcatgaatagggaaattacttctcccttaaggtttttctcaggacaggcctcatacaagaatttcaaggattgcgagtgacatagtttaacattggaccaggcctttcaaattatccaggatgagtttgaaaacacctgtgccactctgctcaacagcagagttttctgtttactaagtattttccctatgctaattacggaaagtttcaacagtttttttaggccaacttatttgatgctagactagacaacttattttttttttcttgcaaggaatactgaaggtaggagtaactaggaagcttaaataaacataaatataaaatgcttatagtgatagaattgacctcagccaattaaaattattaatagaaaaaacatgtcaatgtcaagcctactacctctgttctcacttgagtaatgaggattagtttatatttcccgacaagaatagatgggaattcaaatttcttcctgacctttgttccccctggaacattgggttaggatcatattagaacataaccaaaaagaaataaagattcagacgaattcacaattaatttttaagccccacaaaagtgaaataggtagcattattttttcaagctgtgaaactttccctcattttagtaatagagaaaatgttcagattataaacttggaaactttgctcctaacatatcaattatgccagaggccaatttttaagaagaagagaaatgcatgctctatattctcagcatcatccttgcccacaatagggaaataattttgtaaaatgtttgattttagacctccaaaattatctctatatgctacctgaattaagcaaataaaaaataatatttagaattccatgcaaggcactggtacaattttgtttatcttggcttcattgtttttgaatgtaagatgtacttttaaggcaaataagtacatgttttaagctggtcgcatacagtattggcaatgctataatcacaaatcagaaagtttggaaatgcttacaagtgttaagaggtgtgattcatcatggttatctgaattggcatctgatcttcttttctttctaaatatccctgacatttctgactcctctgtcttttcctcagtaaaactgcaccacacactggaaagcgaagatacacacatttatttatataatgtcaagggagagtaggaataagaagattggccatagacccacccaatcagagtctgggaaatgagaacactttttccttcagcagaaatgctgacgtgccaatgtgaatttagcagaaaaaagatttgccataacttctaagtgagcagccttcagaatgctagcttagattcctggcattaacttgccaggtattttttcaggaaggaaataaattacaattgagcttaaaaacctgagggtagaactcattttcaagcaaatgtgaagcatcagtttgaagttaacaaagttaaagtttggagtagggttcctccagtcctttataatgtagtacaagtattttttttaaatgtataacactagccttttaaattgtattgtgctactaaaagaaattgtgcctgcattcatcttacaacctgggaaccaacgcagagggtctgtggggtagcggtatccagcttcatgccctctgtcctttattgctttctggttagcctgcgtatttcacatacattaaatattccacaataaactctgccatctgtgctgtagggtagtttgtattggtcatgtgctctgtcaagttgacagaggtgcaaagctaaatgtgtgacactcgaagaatatgcatatatttgaataatttgactatttagtccaacaatttgcaaaggcgctctgaatgatcacacattctgataacacttccaaggaacagatagcttcacttagggggtgggggagatggaagcagggttatttctagcaggaattcttgagttcactgaagtcttgtccctggtacttcactgtgtgaacgtgggtaaattatttcctggcagaggatcggattcttcttttataaaacgggtaaataatttctgtcactagtctttagaagttctaaaatagctaatgttagtgaattcattttgctaactgtaaacccttaggtaaattgaactgagtatgtaataatattatatattcagttcaacagcacattcttggtaaccacaagagggtccaggaaaggaaactgtttataaatctttccctttagcaaaattaatgttggagtctttagggaaattcttacagcaatagtcttcgcaattattaggtcaaacccctttgagattacagaaaaacgcacacacacagaaagctgcctgcagaatttgggtgtgggcttggtgggagattcctctgatacccagtgttgtacccccaagagagtgtttctcaaagtgtgacttcagattgtctgcattcgaattgcttgtggtatttattaaaattataactcctgggccctgccccacccctactaaatcacaatttcaggaggagggaccttcattttaacactcacccaggtgatttttatgctccgaggaggtccagggactccaagttaagtacggtactgctgtcttattctttattctaaattttaaggtctgcacaaattggttgaactaatgagaagaaaattcagctttaaagcagaaacacaggtagacggttgacagagttcatcaaatggataattgaaaatgtcctctggaccctagccatataagttctcttcaagggtcttggctacaggcaaatgagaacccgaaaggctatttgctcttttgctgcgggcagtggtgggggtggagggcgggggaggattaactgagccagttctgcccccaccctcgaatcacctacccccactctggttaaagcagaagactttttatttatcttggctgccctggttcgttattaaaagggttagcttatacgtgtgtttgctggggctggaagtgaaaacatctgcaaaagcatgcaatgccctggaacggaactcttctaataaaagatgtatcattttaaatgcgctgaattttgattctggtaattcgtgcactagagtgtctatttcgaggcagcggaggtatcatatgacagcgcacgtcaaggcaccgtggagccctctcgtggactcccacccactttcccattcaccgcggagagggctgctctcgctgccgctccccccggcgaactagcatgaaatctccctgcctctgccgagatcaaatggagcttctcgctgatggggtgcgagtattacctccgccatgcaatttccactatcaataatttaacttctttgctgcagaacagaaggagtacataccgggcaccaaagactcgcgccccctcccccctttaattaagcgaagggaacgtgaaaaaataatagagtgtgggagttttggggccgaagtctttcccggagcagctgccttgatggttactttgacaagtagtgactgaaaaggtgggtttgttttctttctttctctttccgtttttctgtttggtcggctagaaagcgtgtggctttagcgaggtctgtcattgcctgggcttcctggctggaacaagtaacttggtgtaacgttatctgggggcgttcatcaataaaaaatgctgttattatcttgattgaattcctattaggcaaactctagagaggtcagtgcgcgaactctgtttaagccggcgtgtttaaggcagcagagtaaaccaatagcccccatgctctgtgcgatttcattgtgtgctcgcgttcgcaagctccgtagtgcaggaaggtgcgggaaggtgtgtctgtggcccgggaaacgcacgccctctcccagagaacttgggtgctgggatggggaggaaggggagagttgaaagctaggggagcgagacctcggggcgtgcgattctcactcgctccctcccgccccagcgcccacagccggggtttctgcagagggcgcgggacgcggggttccccggggctgaggctggggctggaacacccctcgaagccgcgggcgtcctgtccaaggcgccccaggagggcgcaggactcgcagggcgatgtcgcggggccctaggggaggaggtgaggacaggccccgggggagcggggagttccgggcgcccctcggttccccgcgcgaggaaaagacgcggcgttccctttaagcggccgcctcgaacgggtatcggtagcgcgggcgagcggggagcggggggcggggggcgggggggggggggcggcgccgtttgaccaatcgaagctcaaccgaagagctaaataatgtctgacccgggcgcaaggcgcagcctggagctccgggtccccgacgctgccgccgccgcgcccgggcgcacccgcccgctcgctgtcccgcgcaccccgtagcgcctcgggctcccgggccggacagaggagccagcccggtgcgcccctccacctcctgctcggggggctttaatgagacacccaccgctgctgtggggccggcggggagcagcaccgcgacggggaccggggctgggcgctggagccagaatcggaaccacgatgtgactccgccgccggggacccgtgaggtttgtgtggaccccgaggtaggcaagcgctgggaatggggcttggtgcaggagctgcccgtccgcgggagagagttgactgggggatcccccaccccaaagttgtgggacgaggccagtctccttctttcctcccctccggtagaagggacgatttggagttactcttggggagttttctcccccatcccacaacccagaaggtcagccggcaccaccagggaaaaagggacccggggaagtcacgaagtagaggagggaaggcctggaggagacccagagctgcgtgatgggagcaaagacggcgacccggggatccctcgcagccctcccccagcccaggagtagtcgagagagacttagggggccagagctgtcgagggtcctgactgaggggagggtgctggggctaggctaggaatccttccagggggtgggtggtccccgcgccgacttgcggggggagtgggagggaagcttgcgccttcagcccgcatcccttccccggagctgcacacggctacctgctccccaggaattgagactgaagtggacttacaagtccgaagccaatgtagcttggaaaacttgggaggcggaattcctaccgctgggaactgaaagggtctgcgacactctcgggcaggccgaacccacatctctacccatcctgcgcccctcttctgaagcgccctccagggaagttaagagttttgactttcggggagtggttgggatgtacgtgggggattcttgactcgggttagtctctggggatgcagagccgggaagaggaatgggtgagtgagttactcctggaaagaaatagctgaggattgggggctctgtgcctgacgggcaagaagaaggggagattacagactaggggcatccctaaggaagaagcctcggggctgcgagggtgaactggaggatgcagtgtttgtgtgttgggggtagagcggggatgagggaccggggtggaggggaggcgaggaggaggaggggacccagagaacgaagctagggaaggtagagggtgccctctgccggccatgctgccaagagcagctactgggggcgggaggctgggggtggggaagtggtaaaggaaggttttgcgggatcccttagagagctggtaggagggacttgttgaatggtgctgctgactccagctcggtggggcgtgcgactcgtcgtcggtggattttgactcctcgttcttgtttggcttctatgcaagttttcctcgcgctgggggagctttgataagcctcgattggcggtgtgttagggcttcttggatcttattttagggtcctctagttatcctgcacttactccttaatgtcagtagcaaccaaagaacattttccgacaagcacgcaggaatgttcttggccagaagcaaagaaaggcatatttctgagtgtttattaatcctcctagtaatcttttaaagcaaagtaatatgtaattgggaacgttgattttctaactgcatataaaaggcgacatgatattaaatgagacccctccctactgactcaatatcctgcaaaatctctctctcccctttattattatggaaaaatctatttttatatgagtttgttgtaaggtcaaaagccattttggtcttacaatttgatatgtctttacattttaacttattgaggcataattacagatttaatttgtatgaacgtgtgtgccttcaatgcttatctcatgcaacataatttttaggttggagatttctgatgttatggcatgtagcgtttcaaggcattacacataataggtaacatagcatgttgaaattacaccacaaagttttgaccctgggaacagcaccttttaaaaacaatcactaaactcctgttcctgttttctgattttgcaaatgccttgcttaagacttttttttttttttttttttttttttttttgggaaatttacctctgggttagcaggagaggtaaaaaaaaggaaagagacacttgttgaaatgtaaccataacctttactggaatttaaaacatgttggtcaccattactggaattccagggccataaagtcgttgtcttttttttcttctacttcattttgtaaaatgtgataaatgttggtaaatatagaccagtagtaagtattatgacactaaaagcattatgtatgtggaactattttaagttattacagaacattttctatttataaatgatataagcagaaagaaatgatttccagataaacaaggcttacgtacatgttttgaagcattagaacattgcagacactcttagacatcacattttttaaagcaaaataacagtaatttttcacatacctttggagcctttcatagcccattcagagctgagttagtagctggaagtttcctttattttaaggtgatattttaaaaccatttaacatgtatagtaggtcaacattggtgcatccagaaaatgaagcatttaggaaatctgtttcagtgtcttttcaatgtgtgtaacttttacttgcaaaccaatggaaccaagaaagtcatcatttgcctaaaatgcagtcatcacctcaaatgattcatttatactatgtgagttaattgccttcatctcattaatggccaaggagggaagggaggtcctggggtatttcttgttcattttgactcaccaggagggaaaatcctgtaaaaaaagaaatgcaaatttctaaaatcctggctcaaagtccgtgggtttcctgtttaaaaggggcgccatgaaaatgtaagctattccctttttcctggaatctttaagagtcccagcttttcaatagtcaaaatgtagatgattgatatcatttcttatatgaatagcactggtttgtagttcagcacgcacagtgagctgggcacgcccacctgatagtatagcagagaacttgtttacattctttttacattcatcttctaaaacctggggtgctctctctctctctctctctctctctctctctgtgtgtgtgtgtgtgtgtgtgcgtgcacgtgcgcgtgtgtgtagagggggagagagagagagagagaactgtgaactgtgaaatataacacagccagcagctttgggtctcaatcgtagacttactcttaaggaaatttacagaatggaaaggtcatgttcaagtagtttattaacattttgagatgtaggaaattaatcccggagtacagaagaacaatttcagacttcctgaataaaaacagacagcatagagagtggatgatagctaaactctgaatatcttttgagaagaaaggcactcccatttcaggtgcccataatatggatttgattttagtgattaaaacattaattttcaacttgcatctccctgtgtggaagagttcaatttgtgtgaggggtctcgcctatccaacaaaagtgaatatgtcccttttatagggtaattgctaacttgtctcaacttgttttcaaacaattgttatagagcactcagtttccactaattgcaaaattgttgcttaattgaaggactctcagccatctagtgcagccattcagccactggcaggctctgtgatctcaaactgtgaattgcattttaaagaggaatcgaggagagaattctgtggaattctaggttttaagtgctggctgttgttcaatggaagaggaaatcatttgaacaagaatcgcatcaagttgtgttgtgataaattttctttattaggatgaataacatgcacagatgagcttcaaaagtgaatgagcaaacttactggttacactctgcatccatttactctgtttagtatggagtaatgttaggcaataaatgatgctggcaaatgaaatccgtatgttatttgcatgtggtatttaaacctaggaaacatagagtggctttggtatttgtaggcttagtcatgtgtgtcctaaacgtcctcttaaacttctacttaaggcatagaattatttaatcctaaataattttatacttaagtgcctcactggatttccagaatatttacactgtaaagatttagaaaggtcatgaacccaattattgactatatggaatcattattgatggcagatgcaaaatggagctcactaatgtactgacattgaaaaccttttgcaggggagaggagggggagtggtaaatgtgtgtgttctttaagtggaacaggaaggtattctcttttctgtagaaaaatttgagtatctggtcagataagtgtggaagctttcatttaaattaagtatttaagttcaagtagaagctctagggcacttatcctcttgatgagacaaatcttatcaaatatactagatgctaagaagtggctcattgccctgatgtctcatttatagattgatgtttgaggatgggttgcattaagtgagttagggggctgagtgtgggacaggagaacgattggaaggaagcaaagtaaatttacaagctttagtgacagccataataaagtaaaagtttatttccagagagcctagagagtaaggaacgttatatagttttccccaaaggttcacttgaaagaacttttcattggttgtcatggtagtaatgtcctgattttgaaatctcccagaacctagtagctcttaaacatgctttcatcttggttcctttggtctgacggaaactttatgacgaccctctgtgtttttgacatgcctctgcatttttggagagaggaggtcaggcaagggaggatttcttaaaactaagacagtatagtaaggaaacataaaattatatgataaaaaatcactgaacttcaaattgacttactgaaataaaacctagaaggcaacctgtcgtttaattacaactagcttgtataaaattaaaatttataaaatgggaattcaaagaaaataaacgggcagttccaagtaatttaagcaactcaccaaaaattgaagtaatagtgccacctagagaacaaaatcaccagctttactagccaaatggcttatttccatatgaaccatttttccaacgctacagttactaggatttccttgttaccatattcagatcttgtgagtgtgtatgggggtgggggttgcatgtggaattacagatgaaattttaaaacaagcagatccacaatttgatatatgcactaaatccttttaacgttgtaatgtagccaaatgtagaatagcatgccaggaatcaacggctagcatcctttttaacatttattattttcatggatatgtaccaaaccgaaccattgagtataaaggttctgattttatttatttgctacaggcaattcattatactttctgagatacaataacaccaaataatttgagtagagagacctttaagaatgttttcgatttatgatctacctttaactttaatgtactcagaagatgtgagaataaaataaagtcaaatataagcaagattttaaacacacacacaaaaaacaaacaaacaagaaaaaggaagaaaattataaggattgccttaaccttagaatagatgaaggtatacatctgagccagcaccaaaaaaaaaaaaaaaaaaagttatggaaccaggaaccaataattacaaattgacttaaaattcttggatgacaaaaatctatatttagttcatttttgcatgcgcccacaacagcatccaaaacagttctggggaggcactttgataaatgttgctgaatgcactaatagattgattaatggctgcttcagattatcactagtgatgtagacagaaacttcatgaaaatggtttgtcttgctggaagaaaggcagaaattggaggaaaaggtttaataatatttttccccagtacctattataaaagtcatttagttggcttagttctataatttcttatgtgtaatttgattcacttatgaaattgtgaatatatgaaatgttaaagttgatttagacagcaactataagcttgtggattttcttttaaatgtcttcaaatttttaaatgccagtggagatgccagcgactgtgcttcagggagtagaatatagtatatcttaaatttgtgccaatttctggtaagcagagaaaaaattgcatgataaccaaagaaagtcatattgtttgtgctttgtgttattcatggaagcaatcaggtgcagaaaactttctttttcagaaaaaaaaaattactaaaataaaggtgcgtgtgtgtgtatgcacatatatctaaagggagagagggagaaggaaacttactaaataaaatttttgccacatgggatttagtctaatcagtcttggttttggagttgctatcatcagtagttccattttgtgattctttctttctgccttcatgtgcctttgaaaactgaaactatgcccaaattaaaacaagtttttctgtcttttcacatgttcacttatttcttgaatgtgtttttaaacacagacaaacttcttttacatcatgtagaatctgaaggtcgagaaatttgcagtcattttgctggagagagatgcttggcggagtcccaggccacattcctaggccaaactctcgaaggtattcctcttatgcaacattgggaaaatacatccagcaccgacatgttggctgataatgtgtctgaaggcacagacgatatgcttatcatatgaaacataaagccagcagatattgcagacattctgttgaatgatagaatctggatcatttacatttacttaaatgtaaaatactatgattaagtacaaaaaaatcaatttaggagagaatagagagttgcgggcacggttttaggggatgacttatcagcagattgtagaaaggaagcttgaatgttttaaattaactgcaagttcagtataagccagtggtgtgacaagaggctgttatcatagctactgaaattttgggctgcactgctagaaatataatactgaaatggagaagctaataattcttcactttttaaatagactgtatctagaatattatcatcagttcaaggaaatgaaataagttgttttaggtacatcatcgataaattagtgtacattcaaatcactgtgaccaggatgcatagggaatttgaaagcattgcatgtgagcaatggttgaggggacttggaatgcatgacttagggacaagaaaacttaggctggaatggcaagtggtttttgaatgttgggttgagaagaattctaaaactgtgaaggattagtaaaaataacattcagattgctaatgcctactgtggctgggagattagagtgtcaacatgtgtgatgtatttttgacatccttattttgaggatgggcttcaaagatttgacgaactgtcataagtgtaatttgtgttgcttcagacagcagttctagaaccaatgatgtaaatttagatactctacatggtagttagaaaactttccattaatttaatttagcaaatattgaatgctcactgcatacagagcactttattagaggaatatataataaagaaaaaagaggtctggtgtggtggctcatgcctgtaatcccagcactctgggaggctgaggtgggaggatcacttgagcccaggagatcattacgagtctggacaacatagcaagaccccatctttacaaaagacaaaaaaaattatccaagcctggtgacaggcacttgtagtcccagctacttgggaggctgaggcagtaggatcgtttgagcccaggaggttggggctgcagtgagccgtgattgtcccactgctcttcagcctgggtgacagagtgagaccccgttggagggaaggaagggaaggaaggaagaaaggtaggaaggctgaaatgaaacccttttagaaatgacactaaaatgggaggttggagtaaggtattttctgaagtgcctttgtacttgtttttttctaatgcattggccataagtctgctccttatttatagtccataaacaatcctaatgagaacagttatatatttctgcctttgaatcatctacttgaagtgtttagcatcatgaattgagtatcagaaatccctcccatttctttgcaaagcgctgtattttacttttccttatttgtatacagattctcaaaattggctatttttcctttgggttagacagaacagaatgtctggaaaaaaaagttcttatcaaattcaggtgcccaaattgcttaagaaattaacttttgaggttatatttttttagggttcagtagctaaactaagaaaacttctcaccgttcaccttcacttttggaaaccacaaaatcttcagatattacagttttccaaagagtttctctttttaaatataaactaaaaggaattgactcctcccccaactccctcaggcctcagcatggatagagttactttttttctttaataatttatttataacttattttgctcttctgtagaacagctggagattaagcaacatggccatgacataaaatgcaagttagaccataagatgagcagcccactccaagtatgaatgagtacttattctttgtgatctctcatactgcttttaggtattaatagtgtcagtcagcaaagcaaacagtttaatatttacatctcctttaggatatcatatagtttatagtttgtatgtgttcttgcgtgtatgttttcttctgtttcaaattcttttttcttaaagtaagaatgttatatgtagcaaatggttctttcattaattcatttgttaattcaccgtgcattaattgagtgcccagtgagtgtcaggcactgggctacttggatttcttttccctgtattgatccatatatcttcaggtgctccttgatatggctctccattgactctctcatataaggctttcattacctattcacatactccctcccaaagaggactggtccaaaagtaaaatcttgagcaagttctctgagttatttcagaacttcttgcccccaaactgtatttttaattattgactggtagcattttggaataacttacctctcttttttaaagattgaagttctttatcctctctgattttcaggtgtcagtttgtgtacaaattgagacaataaaaatgtttgttagacattttcttaaagcattttgctatgtgagaatctttcatgaagaactctttttaacaatgactctatagcagaagccacagtagagggagaactactgaatcaaagatggtgtttgagtctatgattttatgatggattttttttttttttacatacaaggatatgcatgggtcttttagtattcaggaatctgttcttcacttgacagtatttataaattgtgtgtttccccctaaaaaaacttaaattgtgagaatgcttccatttactagaagttggttaatgattatgccaaataaggaaaataagacagaaaaatcagtttagtgaatactattttgcctttaaatttagtaatttagtaacagtatctctttggggtttactagaaaccactttttaatccaataggtctctttcattgtgaagtcaggaggtgattttgcttaaatgtgtagtataggaatctatatgtggtgttcaaggatcatgtaaatatgctgatataatcggagcacagtttggcatcattaactcagaaatatttaaactcttgctatacaacatggaagcaaacttgtgcatagtttgtgtgtgtgtgtgtgtgtgaatctcaaaaaaagaaaaaaatcacaggatcaggaagtcggaataggtcccacttttcttctagtaccaaacctacagccatgttcctagccttctcttttactcccaagcaagacagacaggcaaatgaccatcctgctgcccatttctgtgtatattcacttgcattgagagttgtattcacctgcttgttgagagtattcacaaatggtacctgataaagtagatacttctttaaacatgtgaatttttttgcattgtataatgtttagaaataatcatgtataaatggttgaatattaatacaggattgccttatcaagtattttattaatcattaaaatgtggtgtcattaatacaatttattttaagtgcttttcctaaaataccagattatttttctgattttcacatccctgacaatgactttcttaaacttggtagccaggaacagaaaacctaacactgcatgttctcactcataagtgggagctgaacagagagaacacatggactcagggaggggaaccacacacactggggcctggagcagggggcaggggaagggagagagtgcgtcaggacaaacagacaaatagctaatgcatgcgagccttaatacctaggtgatgggttgataggtgcagcaaaccaccatgacacatatttacctatgtaacaaacctgcacattctgcacatgtatcccggaacttaaagtaaaataaaaaataataataaaataaaataaacttagtagcatctattgttccagagcctgtaattgctcttcaggcagtctcacataaaaacctaggagaaccttcactgtcactgttccatgaggtgttaggaaaacttgctctactgcagtgccccagtaggcattggtactgagaccaaaattcagctggtttgttgttactacgattcctacgtgatttcacttgtcatgtagacaagattgcacacttcaataataatcttgtccaaatgtgtggtattccatacatttttaaaatgcattcacatatctcattccatttgatcctacaaataactctataaaaaagattggcagacattatttctatataacagaggaggaaactggagcttagagaagctaaatagcaatccaaaatgcacagctgtagaaccagagcaaggatgatagcccagtgacttcacctaacctagtccccttaccaccactccagctgtctataaccaaaacctgcagtattcaagtaagaaaccatatcttgcccttgatgcattaatgtgagacctggagcaggaacaggctgatattgtcaccctggcctactgtccacctttgtctccagcagagactggtacccttctgtgtgccaaggaataaagtggtaatgggaagattaaaaatgttttttccaaggagttttttaatttaatttttttaaaaaagaaaaaactcttagagggaaaaatgaatatatgacttttgatgtattgttccttagtaacttagttataattttacttaaacctgagactcttgctaagtgaatgattagaaatattaggtggctggccagatggcaaataggaacagctgcagtctgcagctcccagagagatcaatgcagaaggtgggtgatttctgcatttccaactgaggtaccgggctcatctcattgggactggttagacagtgggtgcagcccatggagggtgagccaaagcagggtggagcattgcctcactcaggaagcgcaaggggtcaggggaactccctcccctagccaagggaagccctgagggactgtgccatgagggacagtgctatctggcccagatactacacatttcctacagtctttgcagccggcagaccaggagattcccttgggtgcctacaccaccagggccctgggtttcaagtacaaaactgggtggccatttgggcagacacccagttagctgcaggagttttttctcataccccagtggcacctgaaattccagtgagacagaaccattcactcccccggaaaggggctgaaggccaggcagccaagtgatctagctcagcagatcccacccccatggagcacggcaaggtaagatctgctggtttgaaattctcactgccagcacagctgcctgaagtcaacctgggatgctccagcttggtcgggggaggggcatccgccattactgaggcttgagtaggctgttttcctctcacaatgtaaacaaagccactgggaagtttgaactgggtggagcctaccacagctcagcaaagcccctgtagccagattgcctctctagattctccctctctgggcagggcatctgggaaagaaaggcagcagccccagtcaggggcttatagataaaactcccatctcatgggacagagcacctgggagagggggtggctgtgggcccagcttcagcagacttaaatgttctttgcctgttggctgtgaagagagcagtggatctcccagcacagcacttgagctctgctaagggacagactgccttcttaagcaggtccctgaccctcgtgattcctgagtgggagacacctcccagcaggggtcgacagacacttcatacaggagagctctggctggcatctggtgggtgcccctctgggacaaaccttccagaggaaggaacaggcagcagtctttgctgttctgcagcttctgctggtgatacccaggcaaacagggtctggagtggacctccaccaaattccagcagacctgcagcagaggggcctgactgttagaaggaaaactaacaaacaggaatagcatcaacatcaacaaaaaggatgtccacacgaaaaccccgtacaaaggtcgccaacatcaaagatcaaacatagataaatccacaaggatgaggaaaatccagcacaaaaaggctgaaaattccaaaaaccagaatgcctcttctcctccaagggagcacaactctttgccagcaagggaacaaaactggatggagaatgagtttgatgaattgacagaagtatgcttcagaaagtgggtaaaaacagactcctccaagctaaaggagcatgctctaacccaatgcaaagaagctaagaaccttgaaaaaaggttggaggaattgctaactagaataactagtttagagaagaacataaatgacctaatggagctgaaaaacacagcacgagaactctgtgaagcatacacaagcttcaataactgaatcgataaagcagaggaaaggatatcagagattgaagatcaatttaatgaaataaagcatgaagacaagattagagaaaaaaagaatgaaaaggaaggaacaaagcctccaagaaatgtgggactatgtgaaaagaccaaacctacatttcattggtgtacctgaaagtggcggggacaatggaaccaagttggaaaacactccttaggatattatccaggagaacttccccaaactagcaagacaagccaacattcaaattcaggaaatacagagaacaccacaaagatacccctcaagaagagcaaacccaagacatgtaattgtcagattcaccaaggttgaaatgcaggaaaaaaagttaagggcagcgagagagaaaggtcgggttacccaaaaagggaagcccatcagactaacagtggatctctcagcagaaaccctacaagcctacaagccagaagagagtgggggccaatattcaacattcttaaagaaaagaattttcaacccagaatttcatatccagccaactaagcttcagaagtgaagtagaaataaaatcctttacagacgagcaaatgctgagagattttgtcaccaccaggcatgccttacaagagctcctgaaggaagtactaaataaggaaaggaaaaaccggtaccagccactgcagaaacataccaaattgtaaagaccattgaaactatgaagaaactgcatcaactaatgggcaaaataaccagctaacatcataatgacaggatcaaattcacacataacaatattaaccttaaatataaatgggctaaatgccccaattaaaagaccacagactggcaaattggataaagagtcaagacccatcagtgtgctgtgttctggagacccatctcacatgcaaagacacacataggctgaaaataaagggatggaggaagatctaccaagcaaatggaaagcaaaaaaaaagcaggggttgcaatcctagtctctgataaaacagactttaaaccaacaaagatcaaaagagacaaagaaggccattacataatgataaagggatcaattcaacaagaagagctaactatcctaaacatatatgcacccaatacaggagcacccagattcataaagcaagttcttagagacccacaaagagaccaagactcccacacaataatagtgtgagactttaacaccccaatgtcaatattaggtcaacgagacagaaaattaacaagcatattcaggatttgaactcagctctggacccagtggaactaatagacatctacagaactctccaccccatatcaacagaatatacattcttctcagcaccacatcacacttattctaaaattgaccacataattggaagtaaaacactcctcagcaaatgcaaaagaatggaaatcataacaaacagtctctcagaccaaagtgcaattaaattagaactcaggattaagaaactaactcaaaaccatacaactacagtggaaactgaacaacctgctcctgaatgactactgagtaaataacaaaaagaaggcagaaataaatacattatttgagaccaatgagaataaagatacaacataccagaatctctgggacacagctaaaacagtgtttaggggaaattcatagcaataaatgcccacaggagaaagcaggaaagagctaaaatcaacactctaacatcacaattaaaggaactagagaagcaagagcaaacacattcaaaagctagcagaagacaagaaataactaagatcagagcagaactgaaggagattagagacacaaaaaacccttcaaaaaaatcagtgaatccagaagctggttttttgaaaagattaacaaaatagatagaatgctagccagattgataaagaagaaaagagagaagaatcaaatagacgcaataaaagatgataaagaggatatcaccactgatcccacaaaaatacaatctaccatcagagaacactataaacacctctatgcaaataaactagaaaatctagaagaaatgaataaattcctggacacatacaccctcctaagactaaaggaagaagtcaaattcctgaatagaccaataataagttctgaaatcgaggcagtaattaacagcctaccaaccaaaaaaagcccaggaccagacggattcacagctgaattctaccagaagtacaaagaagagctggtaccattccttctgaaactattccaatcaatagaaaaggagggaatcctccctaactcattttatgagtccggcatcatcctgatacaaaaacctggcagagacacagcaaaaaaataaaattgtaggccaatatccctgatgaacattgatgcaaaaatcttcaataaaaaactggtaaactgaatccagcagcacatcaaaaagcttatctaccatgataatttggcttcatccctgggatgcaaggctggttcaacatatgcaaatcaataaagataatccatcacataaagagaaccaatgacaaaaaccacatgattatttcaatagatgcagaaaaggcctttcataaaattcaacagcccttcatgctaaaaactctcaataaactaggtattgatggaacatatctcaaaataataagagctatttatgagaaacccacagccaatatcatactgaatgggcaaaagctggaagcattcatttgaaaaccggcacaaaacaaggatgccctctgtcaccactcctattcaacatagtattggacgttctagccagggcaatcaggcaatagaaagaaataaagcatattcaaataggaagagaggaagtcaaattgtctctgtttgcagatgacatgattgtatatttagaaaaccccatcatctcagcccaaaatctccttaagctgataagcaacttcagcaaagtctcaggatacaaaatcaatgtgcaaaaatcacgagcattcctatacaccaataatgacaaacagccaagtcatgagtgaactcccattcacaattgctacaaagagaataaaatgcctaggaatacaacttacaagggatgtgaaggacctctttaaagagaactacaaaccactgctcaatgaaataagagaggacacaaacaaatggaagaacattccattctcatggataggaagaatcaatatcgtgaaaatggccatactgcccaaagtaatttatagatccaatgctatccccatcaagctaccattgactttcttcatagaattagaaaaaactactttaaatttcatatggaaccaaaaaacagcccgtatagccaagacaatcctaagcaaaatgaacaagctggaggcatcatgctacctgacttcaaactatactacaaggctacagtaaccaaaacatcatggtactggtacataaacagatagatagaccaatggaacagaacagaggcctcagaaataacgccacacatctacaaccatctgatctttgacaaacatgacaaaaacaagcaatgcagaaaggattccctatttaataaatggtgtcgggaaaactggctagccatttgcagaaaactgaaactggaccccttccttacacgttatacaaaaattaactcaagatggattaaagacttaaacataaaacataaaaccataaaaaccctagaagaaaacctaggcaataccattcaggacataggcatggcaaagacttcatgactaaaataccaaaagcaatggcaacaaaagccaaaattgacaaatgggatctaattaaactaaagagcttctgcacagcaaaagaaactaacatcagagtgaacaggcaaccgacagaatgggtgaaattttttgcaacgtatccatctgacaaaaggctaatatccagaatctacaaggaacctaaacaagtttacaagaaaaaaaacaaccccatcaaaaagtgggcgaagggtatgaacagatgcttctcaaaagaagaaatttatgctgccaacaaacatacgaagaaaagctcatcatcactggtcattagagaaatgcaaatcaaaaccacagtgagataccatcttatgccagttagaatggcgatcattaaaaagtcaggaaacaacagatgcaggagaggatgtagagaaataggaacacttttacactgttggtgggagtgtaaattagttcaaccattgtggaagacagtgtggtgattcctcaaggatctagaaccagaaatatcttttgacccagccatcccattactgggtatatactcaaaggattataaatcatgctactataaagacacatgcacatgtatgtttattgtggcactattcacaatagcaaagacttggaaccaatccgaatgcccatcaatgatagactggataaagaaaatgtgacacacatacaccatggaatactatgcagccataaaaaaggatgagttcatgtcctttgcagggacatggatgaagctggaaaccatcattcttggcaaggtaacacaggaacagaaaaccaaacaccacatgttctcactcataagtgggagttgaacagtgagaacacatggacactgggaggagaacatcacacactggggcctgtcagggtgtaggaggctaggggagggatagcattaggagaaatacctaatgtagatgacaagttgatgagtgcagcaaaccaccatggcatctgtatacctaggtaacaaacctgcacgttctgcacatgtaccccagaacttaaaagtattattattattattataataataataataaaagaaatacaataaaatagaatgcagcatacagcagtgattctcaaacacattcagcatcagaattacccttgaatctttaaaatatatatacatatgagatcttagtctccaagatttgtaagtttggtattgggtccctgggcctatgttgggtttagaaacttctacagatggtttggatgtatgggacagtttaagaatcgctgaactaaaatcaaataaactgaatatcctgtgatttagagagacttatcgtttatttcactatccaagtacttgcattagagcgtggctagaagggatttgcagccttgtaaataatcagaaattcagacattttgagatgagagaactgctgaagattttattctgacttgaaataaattttctaattagaaacttccaggtgagagcaaaggcctggaacaatattcctgagccagaggaggatcgagtttgactccaggcctaacacttactaggtctatgaccttgggtcagtaatttaaattctctgtatctcaacctctcaacagggtattggtagggattaaatgtgttagtgtctgtgaagtgcttagagcagtgcttggcatagtaaatgcttaatgaatttcagccactgtttttatttttagtactttccagctcccccaaaaagatactttttttagacttgtattaagacaataaaaagtttaatcagcatgcttcatacctaaatatgcttcactttatagcaaagtttacaagactaaaactgttttgttgtaattctctgagtctcatgtgtttattaatgattttttctgctgtttattcatctgaattctactcattcttcaagacctagctggaatcctgtttctagaaagactcttgcccataataataaacctgccctatctgagttcctaggtggtctgtacctcataatttggtaattaattgtatatgcacttatataacaaaacattattgtgtgtctttgctgtatcagattctaggctggaagttgtagatatgatgtttttgtctagaaaaatgttctagaatgtcctactcaggacagtctgttgactttaaagacacatttcctaaacagacacttcatgaggcagccccagcctgtacctgtgttcctggacctgatgatcaagtttgatttaagcctcaccacttactagctctgtgattttgggcaagttacttgaattctctgtgtgtagatagaacaatgttgagggaaatccctttcccccatccttgtgtttccacaagggaacttgcttcctaataagtaacactttcaggggaatattctaggcccttctcttatccccattacttgttctttctgtgaaaagaggagaggttaatctgatggatgaaatccttaatctttcatcttctggactgtagagcctgtgaaccaaagcaatggaccacttgcactgaaattgaggctgaccctgtattttgattcttatttggcaacttatttctattctgttcccaattcaaaatcccaaggggagaaggaagataattgattaccagaagtatgtaatggtggtaggaagttgaataaatggtaactttttaaaagttgcatgagatatagtccttatcccagagaagctaagtttgcttttctttcctctcatgtattttagtattatttctacaattagattgtaaaccctttaaaagcaagaatatttctacattttcttactcctgatagcacacagtagactgctgggcacatacatagtaggtggctctgtaggtacttgctaaatgattcaacatgtttttccctcatggaaaagaaagatttcagtattgttcttatcagctaggaaggcactctgaataggaaatcagttctaggcaggtatccataaatgggttatgatttccaacttacttgccccagaggctcgctaatgttgaactcttcatgggtactttgtcttgcttcatgagctatacatgctaaggggttagcagatcatataatcttttgatctacaaaatatgatctttattgaacaaaaacttgggccaaaggcctttctcctttgccaccttcctccctcttttcattctcttttttgggaatgccctttgtgcatgttagttacagcatgtaccacattgcactgtattgttggtttttgggtctaacccacccttaacactgcagtccccaagggcagaaattcagtctcattcattttgatgtcctcagtgcctgtgctcagagaatatctattatttgaaaaaatagtgcaaaagtaaattttaggagactacatcacactcatctaaactgcaagtttgacaagttgacatccaaaagaaaggctctcctaaataacctcgccacagaaatttgggtgacctttgtagctctggagaaagcagaggcaaaaatgaaacctaaaaattatttgtgggtttttaaaaaatgttttctcatggagtaaaggtctacagctgagttcttttcatatgagggaatgacagaaacacagctggttctgactttcagcttcaactgagcgaccagagctctgctggtgaaacaggaacttgtattgtgcccctgacgtgcaccttgaaggtgtcagctcattgtccctttgttcacataaatagttttttaagaattgtttttgatcttgtgagcctctaactaaatgattaaccatgcaaagttggccatttggggtaatactgaagcacttctcttgagggctattgacaggtgggaatgtgcccacctccttgggtctctggttttcatgtcatacttgcaaatcagtgacagtttaaacttggggcaatcacttagcaagtctattgagttaccaagttaattattcccactttgcatgaagcaaccttgaaaatgattttcctaaagcaaagtacatccaaactcagtaccttcttaataacctttgctgaatgaataaatgactaattcataaaaaatgtaacatatctttaattcttacttacgggcagtttaagcctcttgtgtaagaggaggcctcggcttgagataacataggatagtaagcctcctagagaaatttctatatggaaacatggtctgctatgaagctagaagtgagaggacattatatttgaccattatatttggcttcagagcttctcaacatggggcccaaagtcaaggtcccttgtttcattaagaggaggtccaggagtgcatgacacccatcagactactgagacccagctggaactaggcaccttgcacaggggccttgcctaatcaaaatagttcttattttttctgagttccaagtaactagtttcctaacccagtgtctggatagtagtgccaagtgggagtaccttcaatgaacttcctcatgaggttatttctagcctattggaatgtttcgttttaggagggtgaggaagggaagtcttgaatttttgtgcttagtttaatgttgtgatacagctttgaccatccgtttaatgggagatctgttttccagatgactatacatgtggaaaggagaagttttttgagtgttttttttaaccccttttaaagaatggtttttcatttagtctctacatttgggggtaaaaggtcctctagggagacttttcaaaagtatttgaagtttgcatctgatttcagaggtgagttggaggcctatctgtgtatgacagacacatgtctccaacaactatatgttcacaaggactaagagccatccttttgggtccatcattcaacattgatctcacattcgtgttcgtatcagtatctttacagtgcgctcccagttacatctccctaatttcccttagtaggcttcacagaatttgcagtgtatgcaatggcagatgaccacatgtggagtcatttaaccacatcttccactgcaagtcagcccgctcttgatgtctgtttatgtttagattccatcttttggaagatttcattcctctgcactatctcagtatctcagatgcttttgagactgggtccttttcccctcctatgtttggccatggccaccccctcagggttgtgttgtgtttcacagctgctgtttgtagggttgacctttacaatgtacaaagctctttcccatatgttgacaatccctggtgtgatgctgtgagttaggcagggtgtgtatacgtgtcctcatcatattacagtggtaaggcaacagggtttttgaatttgatcacccatgaatttgtctaatttgttggtaaaaaatggtcatgtatcagccgtttcacagggtcagcttaatagaaagtgggagttaggcaggaccagaattcaggacttcagcccccggtcccagggactattctctatacccaattgtcccaccttgaatcagtttcttctagggaaatatctccaaaactgagatggcacccacaggacttcttaattgtagtcattaccaggaaaaacaagcaaaggaactggtgtaaatctctgtttttggtgattggtggagatttggagattgtcttgtgtcaaaagtaaagccactagattaaatgttttgttaataaattggttatttttaatttaattatttgacagttaatttacattattcaaaaatcaaaataaaatttaaaagaagtttacactgaaaagtcttgccccacttataccctgctcacctcagtatcccccaatacataccatctataaggtgatcatttgtattagtttcttgtgaatccttgatagtgtgttttatatagatacaggtaaatatgagtatgtactattatttcccccccaccccaccctgttttttttttgagacggagtctcgctctgtcgcccaggctggagtgcagtggcacgatctcggctcaccgcaagctccaccattttcccccatttttaaaacaaaaggtagtagccatatatacactattttacaccttgttttatcacttactaatatataccagagagctttccatcattttgtacatatgcacctatatctgtcaattattcccagaagtggaattgctgggtcagcaggaaaaatcatgtataattttgataggtattgcctaattgtcctgcacagggcttgaattgtttgtactcccacctttagtgtatgagaagacctgtttctccatagcctcatcaaacagagtgtgtgagattagatgagaaataggaggtgagcagtcttttaccccatccgtagtttgcagtgggaacactgcacagttgcaagagctggtgcaggtatcagattagttccagtggaaacgctgcctcaccatggccatgggcttgcgccagctctagtgacacacacggaatggacccacgttgccacttgcagaatttcctgtagcagaaagttgaacatgcattcattattcatctaactagccatgctggatctaaagagcacaacagtgttttttagaaccaaaaagaaaattgtttcactacaacacactgtgtataaggctttcaatgctcttttctcagctattaacattattttcaggactgagttcaagagatgtatcccaaatcacagggatgtcttgctaagcttggaactttcatactcaagggatgcttttttgaggaatgattttacacttactcaacatttgtaattaaataattagtactttataagataaatttaaactgtccaagtacaatataaacattgaactatgatgcattattgctagactttttccttaaagttgccaagtggtttcctgcattaggcaaataggggatcatataaaaatgccatgatttacggcctagataacatctccaccatttgagcagcatatattccaggtcatccccacataactccttaccattctcattagaaaggttgattcttagtcttatttttctctgaggacagcaaaaaaaaaaatccccttcagttccactgcatagaaaagtgtggtaaatggagccgggcacagtggttatttaatttaaatggacaatattttttatagaattttgacagggccactgtataggggaaagtcactcctcttcccctttatagaagagttgcacctggacagttgcattgatgactgtatccagtctacacaagaggtcattcctgggcataagaatggactgccaaaatctagctgaaacaccattgacaaatagacattttcttttgttaataatacctgtgaaggctttcataacagacatttccagttttgttctcaggctccttgcagctgctcctctaaaagtgtgctctcttccaagagctgacaatggccagaagcaaggtgttctgtcttttgtgccatcatcatctaacttgccacacacatttgggatgtcagcctaggtataggttttgtatccactcagtatggcttgtgggtctggttgcctttgttattcatgctgagggcctctgggcatcagtttggtgtgagagaacccattccatgaccctccttcctttggctgttttgactcgatggctcttgttggcacagtctgtgagtgtctgatgctctatccatgccggaccatctgttctgctgtctctgtggtctgaagtcgttttctgaactattccttgataataaatttgagatgatcttgttctacctttcttttcaagtcacatcttagccccttagccacattcccgaagaacatgacaaatggatgggtcacaagtcacgtagcatagggtgtcagaccacgaggctttgaagggattctgttgggtgctaaaaagaaagattttgtgtcaccacgattttttttaaaggcatgttgacacttaggccttaattgaaagcgttcttactcaagtagagttgacagaggagtatttggtagtcgcggttgctggtctgaagagcatgtggttctgtttcaatgcccaatgagatcttctcacgggaaaatgttctgacatctcaaacaaatgaccttcatgcatagttttgacaaaataccctattaagtatgcatatatggttggtaccttgtggtaataattcaatactggaaacagagtagcaacaaagaaacattagggttatatttaacctctgtggaattagtgtgtaaacaaactgcttatcagaaatgctcatatggggctttgtttaaataaataagaaactggcatatagggtctgcaggatatttctgccaagtagacctccctcacattataagacaccacatctatgtctgaccccatatggaaagaggcatagcaagccagcactggttcatattccctctccaccacataatgggtatgtgatcttagggaatccaccgaaactctctgggcctcagtttcctcagctataaatggtggataatcaaattatttacctcaccattaataaatgttagctattattttttatcaagtttaatacaaagagaaacattttacttatttttccagctatccagagcatcttccaaaatcctatcaccaacaaatactgtattgtatttattatagcaactatgtaaaaatggagtccctgtcctatgcttagatgaaatatgttggtatttgagtttgcatgtcttctataggaatcagtgtttagtgaaaacgggtggagataaacagatgttttcacagtcctgttgttcacagtaccgccaaattgaatgtttccatataggtgcattctaatggcttaaatgatgcagatattttctggccagccatatggatcttttgtcatctaagatgttaatattttccttatattttatagtagttctggagtacagccagtttcttgaatagggtccacatggctcattatgcacagggcctggaaactgccttactcgtgctgttgaaatgaaccgtgacacttcagaagagctgggagctggggtagagcagtggctaggagaacatattcaattatatttcctcctgcattaagctacaagtaatgagcactttcctgtgctttacagttaagtaattaaaagaaattatagagtgggatgcaaaaataacccgaaggacaactggatgtgtggagccaccagttttctccatgagtgcacaaggttaatccttgttactactcagaatgctgagtttctacagaaagggttgcaggtccacacatgttttggcgtctacccacacgcttctgtatggcatgactgtgcatcccagaagaagggctgtgctgtgtacctccacgtttcagtggaatttaacaaactgatccctgaaaatggtttcataaaggtgagtaacagagagctaatagccttctcttgctaattttatctttcccccaagatttcttgataatagtttgaaaaggagtgttattctttggtctctagaggcaacttacctttccagtttcttccatcacctgttttcatctctcttgtttttttaaatttaatgctgtatgtatttcagaggataggatctaatctagtgcggtcccttcatcaggtgagaattattcatctcattttcattttagcccttctgaattaatgacattgaagcccggcagtttggtcctaagatgggtttaattatgtacagatactctttctataatggaaattgctcagataactaattaaccacaagaatacactgtctatggaaaatttcaggagcaccgtctgtggaaaaactgggaagggcatgctgtcaccacagctctggggtctattaaaagtgtggttatgcagcactggtgtctagtggggtgttggctctcaactgccagaattcccatagcatttcatggcagaaagtcaaggtgtccagcaatactctgaaagtgacctgttgattaaagtcgtcaattctgaagaaagagactgaaataagacaaatgggtcttaactttttttctctttctctctcttgtaaaaatgtgtgattgttctggcatgttcccaatccccacataatgccaacatcttttcttaaagggggattccctttatccttggatctgagaattattgcatgttctccctttagggacaatgaatgcagttgcatcacccttgcttttttttttttttttgtacacagcatgcttattcttggatgcagggacttgaaagacaaagccccacctggctttcacaacatctcctattagtaggtgtgccttgtgtgtaatttgaaggaggcggtcccttagctgtgtttacactgtacttttaaatgtggggctgaaggtagaatcaaccatacttaagatgccacctgggaaaatagggttctgtgtcatctcagccccacccatttgcaaatgacttaacagcagcactattagggttcctagtgtgagtcatttgcatttggactggtgaacttggtgacttcttggtgtttggaaacaaacaacctttgcagtctttcgtaaaaagcctgaacagtggaccagtctccagttctacttgcaaagctgcccccatcaaatccctcataatgttcaacttaaaaaatgttacacttttctctggaaatctaaccttttttccttttttaaaagccattttaagtacttcagtcttgaatcaaatgatcccaaatattggacaccaacctagaaattgggttacctcctgggaactttatcgaagaagagagattttggttggagagggggttttgatgtttgatacttatatttactattttaatatttcattgttgttgttgctgctgctgctgtattattttgcgagtttcgtttgtttaaatttcatggtatttggtaggagagagctggatctgttggtttcaggacaagtctagaaataagaaatctgccttgagtgagtgagttggttccctctgttgctatttcaccattaaggacgaaaggaactcacaaggaccagagacatctggctgaaagcaatactagtgtgactggacatctactacctgccatagttggtcatatcgtttccagtatgattctgattgagtgagtgatattaggctatgttcagggatcagggaggctaattatgcttatattgccttgtagcattttggtaagaattaatgattgtgtagatgtccagatttaggtcagcaatattctaaaagttctcattgaactaatcatgtttataagtagcctgtactttctatcataataacaatagtggaaaagctagttgacataaaaggagcccagattttacttaagtaaaaacacaaaagcaaagatattttcccacataaattacaaaagcaaagatattttcccacataaatgtccccataaaacaagttgaaccaaagaggaaagatgacaggtaaccgtatgacacgctaagaaagtatcataatacttaagttaacttcaaccttttatttccttatcctaagcagcctcttttctctttatcatttagtcctgtgcttctcaactttgataagtaaaaaagttattgcactaaataaatcttattgaaatgcaggatctgattgagtgggtggggtaggtggaatgagggtggggaagttgagattctgcatttcttagaagtttctactttatgttaaaatggctaatccatctcaacattgagaagtaaggtttcacttaatttcagcctgtgtaagtttatcccatatgtacatttcctaaaactctaatctcaggccccaggaatttctcctttagttaaaatatttttaggaataaatttgaattgcattaatacacaatttataaatttaacacaaaaaattatttgaagtttgagactttaggttgcatgaaatcaatttcatacttgaaaattttctataaattcaaaagtctgtgtatttaaatacaatttaaatacctgtgttacagtgacatttgtttttctgtctctctctccaccatttccagagtcatcatccctgtacagaaaaatttttcccacatgatttcaccataaattcattaaatatgatgcttacttgataatttctccaggttctttttttttttaattatactttaagttctagggtacatgtgcacaacctgcaggtttgttacatatgtatacatgtgccatgttggtgtgctgcacccattaactcgtcatttacattaggtatatctcctaatgctatccctcccccctacccctactccatgacaggtcccagtgtgtgatgttccccaccctgtgtccaagtgttctcattgttcagttcccacctatgagtgagaacatgcggtgtttggttttctgtccttgcgatagtttgctcagaatgatgtccttgctcactgatggacatttggttggctccaagtatttgctattgtaaatagtgccgcaataaacatacgtgtgcatgtgtctttatagtagcatgatttataatcctctgggtatatacccagtaatgggatggctggctcaaatggtatttctagttctagatcctagaggaatcgccacactgtcttccacaatgtttgaactagtttacagtcccatcaacagtgtaaaagtgttcctatttctctacatcctttccagcacctgttgtttccggactttaatgatcgccattctaactggtgtgagatggtatctcattgtggttttgatttgcatttctctgatggccagtgatgatgagcattttttcatgtgtcttttggctacataaatgtcttcttttgagaagtgtctgttcatatccttcacccactttttgatggggtcatttgattttttcttgtaaatttgtttaagttcttttagattctggatattagccctttgtcagatgggtagattgtaaaaattttctcccattccgtaggtttcctattcactctgatggtagtttcttttgctgtgcagaagctctttagtttaattagatcccatttgtcaattttggcttttgttgccattgcttttggtgttttagtcatgaagtccttgtccatgcctatgtcctgaatggtattgcctaggttttcttctagggtttttatggttttaggtctaacgtgtaagtctttaattcatcttgaattaatttttgtataaggtgtaaagaagggatccagtttcagctttctacatatggctagccagttttcccagcaccatttattaaatagagaatcctttccccatttcttgtttttgtcaggtttgtcaaagatcagatggttgtagatgtgtggtattgtttctgagggctctgttctgttccattggtctatatctctgttttggtaccagtaccatgctgttttggttactgtagccttgtaatatagtttgaagtcaggtagcgtgatgcctccagctttgttcttttggtttaggattgtcttggcgatgcgggctcttttttggttccatatgaactttaaagtagtttttttccaattctgtggagaaagtcattggtagcttgatggggatggcattgaatctataaattaccttgggtagtatggccattttcatgatattgattcttcctacccatgagaatggaatgttcttccatttgtttgcgtcctcttttatttccttgagcagtggtttgtagttctccttgaagaggtcttccacatcccttgtaagttggattcctaagtattttattctctttgaaacaattgtgaatgggagttcactcatgatttggctctctgtttgtctgttattggtgtataggaatgcttgtgatttttgcacattgattttgtatcctgagactttgctgaagttgcttatcagcttaaggagattttgggctgagatgatggggttttctaaatatacaatcatgtcatctgcaaacagagacaatttgacttcctctcttcctatttgaatatcctttatttctttctattgcctgattgccctggctagaacgtccaatactatgttgaataggagtggtgacagaggacatccttgttttgtgccagttttcaaagggaatgcttccagcttttgcccattcagtatgacattggctgtgggtttgtcgtgaatagctcttattattttgagatatgtcccatcaatacctagtttatttagagtttttagcacaaaggctgttgaattttgtcaaaggccttttctgcatctattgagataatcatggtttttgtctttgattctgtttatatgatggattatatttattgatttgcatatgttgaaccagccttgcatcccagggatgaagccaacttgatcatggtggataagctttttgatgttctgctggattcggtttgccagtattttactgaggatttttccatcgatcttcatcagggatattggcctgaaattctctttttttgttgtgtctctgtcaggctgtggtatcaggatgatgctggcctcataaaatgagttagggaggattccctctttttctattgattagaatagtttcagaatggtaccagctcctccttatacctctggtagaattcagctgtgaatccatctggtcctgatggatttttttggttggtaggctattaattattgcctcaatttcagagcctgttattggtctattaagagattcaacttcttcctggtttagtcctgggagggtgtgtgtgtccaggaatttataaatttcttttaggttttctagtttatttgcatagaagtgtttatagtgttctctgatggtagtttgtatttctgtgggattggtggtgatatcccctttatcaccttttattgcatctatttgattcttttctcttttcttctttattagtcttgctagtgatctatcaattttgttgatctttttaaaaaaccagctcctgggttcattgattttttgaaggagtttttctgtctctatctccttcagttctactctgatcttagttatttcttgtcttctgctagcttttgaatgtgtttgctcttgcttctctaaattgtgatgttagggtgtcaattttagatctttcctgctttctcttgtgggcatttagtgctataaatttccctctacacactgctttaaatgtgtcccagagattctggtatgttgtgtctttgttctcattggtttcaaagaacatctttatttctgccttcacttcgttaagtacccagtagtcactcaggagcaggttgctcagtttccatgtagttgagtggttctgagtgagtttcttaatcctgagttctagtttgaaagcactgtagtctgagaggcagtttgttataatttctgttcttttacatttgctgaggagtgctttacttccaactatgtagtcaatttttggaataagtgtgatgtggtgccgagaagaatgtatattctgttgatttggagtggagagttctgtagatgtctattaggtccgcttggtgcagagctgagttcaatttctggatatctttgttaattttctgtcttgttgatctgtctaatattgaccgtggggtgataaagtctcccattattattgtgtgggagtctaagtctctttgtaggtctctaaggacttgctttgtgaatctggtgctcctgtattaggtgcatatatttttaggatagttagctcttcttgttgaattgatccctttatcattatgtaatggccttctttgtctcttttgatctttgttggtttaaagtctgttttatcagagactaggattgcaactcctgcttttttttgctttccatttccttggtagatcttcctccatccctttattttgagcctatgtgcgtctctgcacatgagatgggtctgctgaatacagcacactgatgggtcttgactctttatccaatttgccagtccatgtcttttaactggagcatttagcccatttacatttaaggttaatattgttatgtgtgaatttgatcctgtcattatgatgttagctggttattttgctcgttagttgatgcagtttcttcctagcctcaatgatctttacaatttggcatgtttttgcagtggctggtactggttgttcctttccatgtttagtgcttccttcaggagctcttgtaaggcaggcctggtggtgacaaaatctctcagcatttgcttgtctgtaaaggattttatttctccttcacttatgaagcttagtttggctggatatgaaattctgggttgaaaattcttttctttaagaatgttgaatattggcccccactctcttctggcttgtagagtttctgccgaaagatgctgttagtctgatggacttccctttgtgggtaacctgccctttctctctcgctgcacttaatgttttttccttcatttcaactttggtgaatctgacaattatgtgtctttgagttactcttcttgaggagtatctttgcggcattctctgtatttcctgaatttgaatgctggcctgcctcactagattggggaagttctcctggataatatcctgcagagcgttttccaacttggttccattctccccatcactttcaggtacaccaatcagatgtagatttggtcttttcacatagtcccatatttcttggaggctttgttcatttctttttactcttttttctctaaacttctcttcttgcttcatttcattcatttgatcttcaatccctttcttccacttgattgaatcagctactgaagcttgtgcatgtgtcacatagttctcgtgccatggttttcagctccatcaggtcatttaaggtcttctctatgctgttttttctagttagccattcgtctaatgttttttcaaggtttttagcttctttgctaaaaggttcaaacatcctcctttagctcggaggagtttgttattactgatcatctgaagccttcttctctcaacttgtgaaagtcattctctgtccagctttgttccattgctggcgaggagctgcattcctttggaggagaagacgtgctctgatttttagaattttcagcttctctgctctggtttctccccatcttattggttttatctacctttggtctttgatgatggtgacgtacagatggggttttggtgtggatgttctttctctttgttagttttccttctaacagtcaggaccctcagctgcaggtctgttggagtttgctggaggtccactccagaccctgtttgcttgggtatcaccagcagaggctgcagaacagcaaatattgcagaacggcaaatgttgctccctgattgttcctctggaagcttcgtctcagaggggcacctggccgtatgaggtgtcagtcggcccctactgggaggtgcctcccagttaggctactcaggggtcaggaacccacttgaagaggcagactgtccattctcagatatcatattccatgctgggaggacccctactcttttcaaagctgtcagacagggacatttaagtctgcagaagtttctgctgtcttttgttcagctgtgccctgcccctagaggtggagtctacagaggcaggcaggcctccttgagctgcggtgggctccacccatttcgagcttcctggctgctttgtttacctactcaagtctcagcaatggtggacacccctcccccagcctcgctgctgctttgcagttcgatctcagactgctgtgctagcagtgagccaggctccgtgggcatgggaccctccgagccaggcctgggacataatctcctggtgtgccgtttgctaagaccattggaaaagcacagtattagggtggggagtgtcctgattttccaggtaccgtcagtcatggcttcccttggctaggaaagggaattccccaaccccttgtgcttcctgggtgaggtgatgccccaccctgctttggctcatgctccgtgggttgtacccactgtctgacaagccccagtgagatgaacccggtacctcagttggaaatgcagaaatcacccgtcttctgcatcactcacgctgggggctgtagactggagctgttcatatttggccatcttggaacctccctttccaagttctttattacagagtgggtcactgaaacttcatggaacaaattggaaattatcttcttaattaatgtcactgtctaccatgtatgggaatttggtaaatattatatggtttcaataacatagtagatagaacattgtcaaatctaaacttcagtgaattgtaacagatcccacctgaaattctaaagaaaacagaattctaattgaagaggttaaacttttacagggaatgtcaactgccatttgggtcctgtaaacaaaaaactgttttttaaaaaagtaaactttaaaagtattttcagatgacctcatttgctatccaagtggcttgagtatgcttgatgctaagacttctttgttacagactggagatgtgtgctactggggcagtgttgctctgtgacaaggaggcagaggatgagggcaaggttcgatgtgactgtgaattctgggtggctctggctatcgggagccttcattgattacagcaaaacagttgctttcctagggcaatagtgtctctgtcacccaggctggagttcagtggcatgatcaatcgctcactgtagcctcaacttcttagactcaagtaatcctcccacctcagcctcccaagtagctgaaactacaggtgtgcaccaccacacctaatttttttaatttttaagtttttgtagagacatggtctcactgtgttgcccaggttgatctcgaattcctgggctctagtgatcctcccgcctcggcctcccaaagtgttgggattacaaatgtgagccactgcacctggccctttgcaaccttcttgacaatgcattcctttattccctaactggaagtaacttctttctctttataaaattgtatctgtaccttttctgggtcatttctacctttatattctagttacgtatgtcctacctccctcctagggagggaggtaagtaagactggaaagtagacttcatgtgtgatgaatgaatgaacaaaaggaagtctaacatatggatatagtcaactggatgcaaattaaaaatttttaaatattgatttgcaagatttcattaaggtcaactcttaatagtttgtatcatatatgttaggaaccaaatattaataacttcttcagcattaccattatctttataggactgtctaaaatgagcagccatatctttaaactgtgttttctctgattacacgctcacaggtaaaacccaaaggggctgggaacaaacaagacttttttttttttctgtatgcctgaattatctgtactgttgcttgttttcccacctttggccatagaaacttagttctaacatgctacaatttttgcagttctttctcttagaaaaagaccacattgtctgaaatttcatccatttaagtaatcaagccttaaagttgaaggatcttggtcatgattaatctagacctacaaagtagtatcttaatggcactccttttagaaagttaggttccaggacacacatagctgcagtgtccacattttgtaagctccttcgttgtcacagccactctcttctctgtggctgatattctaaaactggcaacacatcctgatggtaaaagcttggttcaggagacaggtgacctactagctttatggcatttgacaggttacctaacctctctgacgcataattgcctcatctatataatggggataataatacccatcctgtctccttgtaaaaatcaaattagatgacgcctgtgaatgttctatagtctcttagacaaatgtaagttatgactacagcaagagtaaaagagcatgttgttatggacattctttcagtgaaatgtctaagacttgtgagtcacacttaaagctaaacttgatatctacttcattgattttctttttagttctatgtactatattgaatttcctgacagtggggctatgaaagccttcctagcattttatagatgtggttgaattaatggctgtaagccttaaagcagaattagacagcatcaatgaatttattaagtataaataaatatataatctgcttagcaatattacacagcctctttatcttatgtgtgataaagagtcatccgaaggttgaaaatgaagaattgtcctggaagctcttacttaatcttttattatttcctaatacagtatataaaattactcattgaaagcttagcagaataagaaacaagaagttaaaaggctgaaaactacaaattttgctattattattgttattacttcccaagtctcttattgatctgttagaaatagagctacacaggaaattgtaggacagttagtatgtggtagtgttatctgctttttaattattcaagtaaggttttattccattagaggaactcaagaagttggtcatggctgataattgctatctgtcaaattccttagagcagggatccgcaacacccaggccatggattgttaccagtccctggcctgttaggaaccaggctgcacagtaggaagtgagcggcgggtgagcaaacattgccatctgagctctgtctcctttcagatcagcagcagcattagattctcatagaagcatgagccctgttgtgaactgcacatgcaagggatctaggttgtgtgctccttatgagaatccaattccttatgataatttaactgatgatctaaggtggaaccatttcatcccaaaaccatccaccctgctactcccagaccgtggaaaaattgtcttccacaaaactggttcctgctgccaaaaaggttgggaccactgccttagagtttataatttggggttagcacagcctatatttacctgagaatttcaatgggttcactgatctttccaaatgaaaaggcttcttacgaaaattatatccaaactgtcttttctcttagtttaataaacctatcagtaagtttttactgagtactgctattacatttttctctgttaagcattatgggggctcagacatgatccattccctcaaagaacttacctttcagctgaagactgactagaatgagcaaatacggttaacaattaacaagtgagtaggccagctcggccaacatggtgaaaccctgtctctactaaaaatacaaaaattagccgggcatggtggtgggcgcctgtaatcccagctacctcctgctgaggcaggagaattgcttgaacccaggaggtggagattgcagtgagccgagattgcaccattgcactccagcctgggcgacagagcaagactctgtcttgggaaaaaaacaaaacaaaacaagtgagaagggaatcaagtactacgtaagatgtaatgtggaattttagggaaggaaggcagtgtatgctggagtaattagagaaaggggcatgcatgatttgatgcttgaactggatcatgaaggataagcaagattttggcagcaagtgagagggagagaggagtttgtcagaggaatggaacaagtcaggaggcagtaatgtgtacggcactccaaggactctgtcctgatcggagcagaagtgatgaagcattgagtagtttgagaaagttagctaagaagggtgagggcagatgttggagagagttgagtatcagagagaagacattagatttgagcagatagaacaaaaatgccattgccagtttttgtgtaagagaatagtattagggtggttacccaggaagtggtcatcagagttgaatggagcagaaagagtgtctgatacagcatcgggacgttgtggtcacaaaatgaggtggtgagggctgagccaagatggtggcagtgggatggataaaaagggatggccaggacaaatattttaaaggaaaaattaacaggacatctttactgactggattggaaggctatgcaggaaatatattgtcaaacttgattccaggatttctatcctatgcctgggttgcccaaaatatcagggaaccattgttagaaaaggtaggagataccactgttccaacaaaaagtattgagtttggtgttgcacccacttaacttcaaggccttacaagtgagtagacagttagttagaattgcagaagtgccactcagagagcagggcttgcaatgtggggttggactttgtcaccattgtgttaattcctaattctatgcagatgctcagcttgaggaatacccatgtttgggcttcagaatgaaagccaagtaatatttactcagatgccaattttccctctgaaatatttgctcatggaactgagagaacaatatataaagcattaattatttttctcataaagttattaataaaaagataagatcagtgaaaggcagagtaaactagaagccaagtatagaaaatggtatcattcaaagactcattactgtagtggtgaaaacaaaacaattttccaacagcttaagatgcctcagtattttggaccatttttaagtagttagtgtgggcacttagtaaatatgtattaaactatagttcattaattcttttttttttttttttgagatggagtttcactctggtcacccaggctgcatttttgctttcttagtgatatataaaatgtcgagtttcacaatgatggtatcttagatttgattaaatatggtattaaaaaatagctgatcacagaaagtctctaccagtgtgatgtagatggctaaagtattccacatttgcaaacttttattgacctaaataagaggtgccccttgggttgtttttatttggactgggaatattaggagaaagctttttcattcagtgtgtaagtacaatctaccagaaatagaaacccccatggacgatctatttctttgatggtacaggactcagaacattcacaaagatttagttgttagcggaatagacatctgtattttattcaaaccaattttcccttcctaatctgagaacattgtgcaatctaagcagttctaagcatgtttgctattcgtgcaaagtgagagtaaatctaaaagaaatttttttgtgtgtttagggatggtaataaagtctcttagtggttgaaaatgttatttcttacaaaagtggagaacatttgcttttcaataccagagttttcagccatttctgcattctgacctattgactggaggtaggttgcctttgaattcagtaaaacttcatgggcagaaacacagttccttttcctacttatttggatatcatgatggccattgcatgtatgtgtctttttgtaagtccatgcctcagaactgagaagtaggaataaaattagggtcagggctggggatgctactctttgctgctgagaaacacaatgcttcaggtaagtgattctgaagtccttcaccacctgacggtaaccttgggttggtccataggtatgttttcattttgcttgttcatccattttaattggcttcctagagcatgcttgtagatgtagagccaaatttagagtagagcaaccctctggcaaacaggaagagattaattttgtggtatgcttttaagggacttcccaggaaacttcaaaagcagaaaaagaagcactagctgcctattccaaaatgtgtaaaacaccactcagctttttaaaagtaggataaactcagagcgcgcgcacacgcgcgcgcgcacacacacacacacacacagagagaacatctctagtaaaaagaaaagttgagctttcttagctagatgtgtgtattagccagaaaaagccaaggagtgaagggttttagagaactggaggagataaagtggagtctgcatatgggaggcatttgaaatggacttaaatgtctttttaatgctgactttttcagttttctccttaccagacacattgttttcatgacattagccccaggcatagacacatcattaaaatgaacatgtcaaaaaatgatttctgtttagaaataagcaaaacattttcagttgtgaccacccaggtgtagaataaagaacagtggaattgggagccctgagttctaacataaactttcttcatgacataaggcaagtcttctatggcctttggtttccttacctgtaaaacaggatggctcaatgaaattatctttcttctttgctataatagagtatctctgtgggaagaggaaaaaaaaagtcaatttaaaggctccttatagttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggtttatatgctagatcttgggggaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaa

SEQ ID NO: 36 - protein, 280 aa, Homo sapiens
MPADIMEKNSSSPVAATPASVNTTPDKPKTASEHRKSSKPIMEKRRRARINESLSQLKTLILDALKKDSSRHSKLEKADILEMTVKHLRNLQRAQMTAALSTDPSVLGKYRAGFSECMNEVTRFLSTCEGVNTEVRTRLLGHLANCMTQINAMTYPGQPHPALQAPPPPPPGPGGPQHAPFAPPPPLVPIPGGAAPPPGGAPCKLGSQAGEAAKVFGGFQVVPAPDGQFAFLIPNGAFAHSGPVIPVYTSNSGTSVGPNAVSPSSGPSLTADSMWRPWRN

SEQ ID NO: 37 - DNA, 843 bp, Homo sapiens
atgccagctgatataatggagaaaaattcctcgtccccggtggctgctaccccagccagtgtcaacacgacaccggataaaccaaagacagcatctgagcacagaaagtcatcaaagcctattatggagaaaagacgaagagcaagaataaatgaaagtctgagccagctgaaaacactgattttggatgctctgaagaaagatagctcgcggcattccaagctggagaaggcggacattctggaaatgacagtgaagcacctccggaacctgcagcgggcgcagatgacggctgcgctgagcacagacccaagtgtgctggggaagtaccgagccggcttcagcgagtgcatgaacgaggtgacccgcttcctgtccacgtgcgagggcgttaataccgaggtgcgcactcggctgctcggccacctggccaactgcatgacccagatcaatgccatgacctaccccgggcagccgcaccccgccttgcaggcgccgccaccgcccccaccgggacccggcggcccccagcacgcgccgttcgcgccgccgccgccactcgtgcccatccccgggggcgcggcgccccctcccggcggcgccccctgcaagctgggcagccaggctggagaggcggctaaggtgtttggaggcttccaggtggtaccggctcccgatggccagtttgctttcctcattcccaacggggccttcgcgcacagcggccctgtcatccccgtctacaccagcaacagcggcacctccgtgggccccaacgcagtgtcaccttccagcggcccctcgcttacggcggactccatgtggaggccgtggcggaactga

SEQ ID NO: 38 - DNA, 1475 bp, Homo sapiens
gggatcacacaggatccggagctggtgctgataacagcggaatcccccgtctacctctctccttggtcctggaacagcgctactgatcaccaagtagccacaaaatataataaaccctcagcacttgctcagtagttttgtgaaagtctcaagtaaaagagacacaaacaaaaaattctttttcgtgaagaactccaaaaataaaattctctagagataaaaaaaaaaaaaaaaggaaaatgccagctgatataatggagaaaaattcctcgtccccggtggctgctaccccagccagtgtcaacacgacaccggataaaccaaagacagcatctgagcacagaaagtcatcaaagcctattatggagaaaagacgaagagcaagaataaatgaaagtctgagccagctgaaaacactgattttggatgctctgaagaaagatagctcgcggcattccaagctggagaaggcggacattctggaaatgacagtgaagcacctccggaacctgcagcgggcgcagatgacggctgcgctgagcacagacccaagtgtgctggggaagtaccgagccggcttcagcgagtgcatgaacgaggtgacccgcttcctgtccacgtgcgagggcgttaataccgaggtgcgcactcggctgctcggccacctggccaactgcatgacccagatcaatgccatgacctaccccgggcagccgcaccccgccttgcaggcgccgccaccgcccccaccgggacccggcggcccccagcacgcgccgttcgcgccgccgccgccactcgtgcccatccccgggggcgcggcgccccctcccggcggcgccccctgcaagctgggcagccaggctggagaggcggctaaggtgtttggaggcttccaggtggtaccggctcccgatggccagtttgctttcctcattcccaacggggccttcgcgcacagcggccctgtcatccccgtctacaccagcaacagcggcacctccgtgggccccaacgcagtgtcaccttccagcggcccctcgcttacggcggactccatgtggaggccgtggcggaactgagggggctcaggccacccctcctcctaaactccccaacccacctctcttccctccggactctaaacaggaacttgaatactgggagagaagaggacttttttgattaagtggttactttgtgtttttttaatttctaagaagttactttttgtagagagagctgtattaagtgactgaccatgcactatatttgtatatattttatatgttcatattggattgcgcctttgtattataaaagctcagatgacatttcgttttttacacgagatttcttttttatgtgatgccaaagatgtttgaaaatgctcttaaaatatcttcctttggggaagtttatttgagaaaatataataaaagaaaaaagtaaaggcttttaaaaaaaaaaaaaaa

SEQ ID NO: 39 - protein, 166 aa, Homo sapiens
MAPSTVAVELLSPKEKNRLRKPVVEKMRRDRINSSIEQLKLLLEQEFARHQPNSKLEKADILEMAVSYLKHSKAFVAAAGPKSLHQDYSEGYSWCLQEAVQFLTLHAASDTQMKLLYHFQRPPAAPAAPAKEPKAPGAAPPPALSAKATAAAAAAHQPACGLWRPW

SEQ ID NO: 40 - DNA, 501 bp, Homo sapiens
atggcccccagcactgtggccgtggagctgctcagccccaaagagaaaaaccgactgcggaagccggtggtggagaagatgcgccgcgaccgcatcaacagcagcatcgagcagctgaagctgctgctggagcaggagttcgcgcggcaccagcccaactccaagctggagaaggccgacatcctggagatggctgtcagctacctgaagcacagcaaagccttcgtcgccgccgccggccccaagagcctgcaccaggactacagcgaaggctactcgtggtgcctgcaggaggccgtgcagttcctgacgctccacgccgccagcgacacgcagatgaagctgctgtaccacttccagcggcccccggccgcgcccgccgcgcccgccaaggagcccaaggcgccgggcgccgcgcccccgcccgcgctctccgccaaggccaccgccgccgccgccgccgcgcaccagcccgcctgcggcctctggcggccctggtga

SEQ ID NO: 41 - DNA, 1319 bp, Homo sapiens
cgcgcttggccttgcccgcgcccgctcgcctcgtctcgcccggcctccccgcgtcgcctcgtcgcctgttccgcgccaggcatggcccccagcactgtggccgtggagctgctcagccccaaagagaaaaaccgactgcggaagccggtggtggagaagatgcgccgcgaccgcatcaacagcagcatcgagcagctgaagctgctgctggagcaggagttcgcgcggcaccagcccaactccaagctggagaaggccgacatcctggagatggctgtcagctacctgaagcacagcaaagccttcgtcgccgccgccggccccaagagcctgcaccaggactacagcgaaggctactcgtggtgcctgcaggaggccgtgcagttcctgacgctccacgccgccagcgacacgcagatgaagctgctgtaccacttccagcggcccccggccgcgcccgccgcgcccgccaaggagcccaaggcgccgggcgccgcgcccccgcccgcgctctccgccaaggccaccgccgccgccgccgccgcgcaccagcccgcctgcggcctctggcggccctggtgacccggcgggacctgcgggcgcgcggcccgacgaccagagggcgagcctgctcctctcgcctgtagggaagcgccttcccgccgtcgtccgccccgggcttggacgcgcccttctccggaaggctctggccccaagctggccggcccgcaggagccccattctcagagaatgtgtgtgcagagtccctgccgttttaggacaatcagggcccatcttctgccaagtgtctgaccccatggggttgttctgtgtttgcatttaagcaagtgacttctgggaagtccccggccgcccggggttctatgatatttgtagtgccggggctcgcacactgctgcccccagcctgtagaggactttcttcagggcccgtagctgctgggcgtacccctggcaggcgggctgtgccgcgggcacatttgccttttgtgaaggccgaactcgagctgtatcctcataggaaacagtgatcaccccggacgggcgtccaggaccctgagggccatggccaaaaggctcctgagtgtgcctggtggtctggctggggctcacggtgggctgtctggggagggtgggtgcctccactatgatccttaaaggattcctctgtgtgggtggatgcgtgtgggcacgactttgtactcagaaattgaactctcagtcacgtggaagccacgggactgctccgaagccgccataataaaatctgattgttcagcccccaaaaaaaaaaaaaaa

SEQ ID NO: 42 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 43 - DNA, 597 bp, Homo sapiens
atgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaa

SEQ ID NO: 44 - DNA, 5114 bp, Homo sapiens
ttaaggccgcgctcgccagcctcggcggggcggctcccgccgccgcaaccaatggatctcctcctctgtttaaatagactcgccgtgtcaatcattttcttcttcgtcagcctcccttccaccgccatattgggccactaaaaaaagggggctcgtcttttcggggtgtttttctccccctcccctgtccccgcttgctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggctgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacggtaatgaccctttcccaaccatagaatgtgtttggggccccgctttgcctgctggagggtgttaaccttagcttgcttttcggcgtattctgatttagctttgggagagctaactttattggtcttaggtgttcagtgctacctggcccactgcttgtctgtttgtgacttttaagtcagaaactggagatggtaagatccgataatttccctaacttaatacatcgcggtccctctcactagcaactcctaggtatgtgacaaagttgggatgtttatcaacggtccgcctcctggctagggaaagagctctggggcggagaatgcactttctgttttttgaaaacaacctcattttgtgcccttaaaagccactggggatgacggatccaggattgtgggtggaggtagtgggtttttcatcccctgactatggggccaacttctgccagccattgttttttctaataaagattgtgtgttctttttaaaaatttcccctgcgcttagattcttctactcaaaacaaaagagccaacagaacagaagaaaatgtttcagacggttccccaaatgccggttctgtggagcagacgcccaagaagcctggcctcagaagacgtcaaacgtaaacagctcggtgggttgatcactaaaggagcacgcactggaacccggggccttcagacctcacgatacctgatcttactggttgctggcaaattaaaagcttatggggttttgttttgtttatacttcgtgaggtcaaaaaagtagcaatggggaaggctggggatacggtaattcctcagagtttctatgcccagagatactttctcttcaaactgttgaccagagcagctacttgtaacccaggccccatcgggtaggaaggtcgtttccctgtgagtcccactaaaacgtgttgggagcaataggttctttgcccatccgaacaagaactagggtactccctcagtccgaattaatgagaattaatttcctagaggttcagcttgagtcggtaacagattttgagccatacatggaaaaatggcaaatacatgattaagtttcaattttgagggggaatgtttggtagaaattgctcatctttggttatgcaagggattagagatgtgaataggatggtatgttgtgttctttgacattttaataaactgtcactttccctgttgtctcctaagtttggagagagaaggaaccagtatttgcaaaaaccaaatggaaagataaaaaagttactaaagtttctacagaatttctggtaacactgaagttgcaaagcagaagttaaattaactcttgtcagtaagcaatccaggaacacgtcagccagtgtatgctaattgtgccgtaacagggtgatttggatatttgtaggggaaatgggtagtaaatatcaagactggtgaccgtaggtcagcccagcacaaaggaagtggagatttttccatgcacaagaatctgatcactgtaaatagctaatttgaataattcagtccccagataaccaacatgggttggttattcataataaactacatattttaatagtttattagcttcctttagaccaagactgtgacctctttattttctaaagcacacacgtagtttagcatatgaggcgataaaatattgatgttaactttttaaatccccagttataaaaattttaaaataacagggattaaggtgagattcaggtttgttgtgtctttaaattgtatatgtgacttcacatatctttttcagcgcttatacaaaacggcactatagaacctccattttacagcaccatatgaagtgggaaaattaggtgaaaattttcctgaagcaaccttaacatgcgcagcccttgttggtttgtgacttgtggcctagctcatcagatgagccacgagaatcagacctggattttgatctggccctgttctgacatgcaatgaggcatttgtaggatttagtaatattgctagttcaaagaatactagaaatattagtaagaacctattcaaaagtattcatgagtattttctgcatatgaatcaggaattagaatattttgaaaatgatgttaataaaattttcctctggaaggcctttataatttttattcccaatcatttttcaaatttagaaagtttaatctgtcacaggagaaaaaaaattaaaaattttcaaaaatttagaaaatttttacccgtaagtattacagtttcctaattatcctatttatttcccacttgcctttgacctagattatttagagtagggtttctcagcctctgcactaatgacattttgggccgaataattctgttgtaggaggctgtcctgtgtgttttagattgtttggaattatccctggcctctcacactagatgccagcagtatcctcctcccccagtgtgacaacctaaaatgtctccagacattgccaaatgttccctgtggggcgggggcaacattgcctactgttaagaactactgctctagaccaaagaacacagcacagaggaaaggaaaaaaaaatccagttaagagaatgttaggtggagatgactatagtcatcaaacttttttccccatcaagtatttccaagctaacatagtgacaaaataattcctgtactctactggtaacgttaatctagtgctcttcctttaattcttccgttttgttttcttttgcagaattaagaatatgtttccttgtttatcagatacatcactgcttgatgaagcaaggaagatatacatgaaaattttaaaaatacatatcgctgacttcatggaatggacatcctgtataagcactgaaaaacaacaacacaataacactaaaattttaggcactcttaaatgatctgcctctaaaagcgttggatgtagcattatgcaattaggtttttccttatttgcttcattgtactacctgtgtatatagtttttaccttttatgtagcacataaactttggggaagggagggcagggtggggctgaggaactgacgtggagcggggtatgaagagcttgctttgatttacagcaagtagataaatatttgacttgcatgaagagaagcaattttggggaagggtttgaattgttttctttaaagatgtaatgtccctttcagagacagctgatacttcatttaaaaaaatcacaaaaatttgaacactggctaaagataattgctatttatttttacaagaagtttattctcatttgggagatctggtgatctcccaagctatctaaagtttgttagatagctgcatgtggcttttttaaaaaagcaacagaaacctatcctcactgccctccccagtctctcttaaagttggaatttaccagttaattactcagcagaatggtgatcactccaggtagtttggggcaaaaatccgaggtgcttgggagttttgaatgttaagaattgaccatctgcttttattaaatttgttgacaaaattttctcattttcttttcacttcgggctgtgtaaacacagtcaaaataattctaaatccctcgatatttttaaagatctgtaagtaacttcacattaaaaaatgaaatattttttaatttaaagcttactctgtccatttatccacaggaaagtgttatttttcaaggaaggttcatgtagagaaaagcacacttgtaggataagtgaaatggatactacatctttaaacagtatttcattgcctgtgtatggaaaaaccatttgaagtgtacctgtgtacataactctgtaaaaacactgaaaaattatactaacttatttatgttaaaagattttttttaatctagacaatatacaagccaaagtggcatgttttgtgcatttgtaaatgctgtgttgggtagaataggttttcccctcttttgttaaataatatggctatgcttaaaaggttgcatactgagccaagtataattttttgtaatgtgtgaaaaagatgccaattattgttacacattaagtaatcaataaagaaaacttccatagctattcattgagtcaaa

SEQ ID NO: 45 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 46 - DNA, 954 bp, Homo sapiens
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 47 - DNA, 2513 bp, Homo sapiens
ggatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaa

SEQ ID NO: 48 - protein, 159 aa, Escherichia coli
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 49 - protein, 112 aa, Arabidopsis thaliana
MGVEKQVIRPGNGPKPAPGQTVTVHCTGFGKDGDLSQKFWSTKDEGQKPFSFQIGKGAVIKGWDEGVIGMQIGEVARLRCSSDYAYGAGGFPAWGIQPNSVLDFEIEVLSVQ

SEQ ID NO: 50 - DNA, 1805 bp, Artificial Sequence
  Description of Artificial Sequence Synthetic polynucleotide
  pITR-CMV-mScarlet
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatgcctaagaagaagcggaaagtcggctccggcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - DNA, 130 bp, Artificial Sequence
  Description of Artificial Sequence Synthetic polynucleotide
  5′ITR cDNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 52 - DNA, 328 bp, Artificial Sequence
  Description of Artificial Sequence Synthetic polynucleotide
  CMV enhancer
ctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 53 - DNA, 237 bp, Artificial Sequence
  Description of Artificial Sequence Synthetic polynucleotide
  CMV promoter
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgc

SEQ ID NO: 54 - DNA, 33 bp, Artificial Sequence
  Description of Artificial Sequence Synthetic oligonucleotide
  SV40-NLS
atgcctaagaagaagcggaaagtcggctccggc

SEQ ID NO: 55 - DNA, 696 bp, Artificial Sequence
  Description of Artificial Sequence Synthetic polynucleotide
  mScarlet
gtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaa

SEQ ID NO: 56 - DNA, 240 bp, Artificial Sequence
  Description of Unknown Bovidae bGHpA sequence
gctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 57 - DNA, 141 bp, Artificial Sequence
  Description of Artificial Sequence Synthetic polynucleotide
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 58 - DNA, 2285 bp, Artificial Sequence
  Description of Artificial Sequence Synthetic polynucleotide
  pITR-CMV-mScarlet-DD
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatgcctaagaagaagcggaaagtcggctccggcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 59 - DNA, 483 bp, Escherichia coli
ggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataa

SEQ ID NO: 60 - DNA, 3440 bp, Artificial Sequence
  Description of Artificial Sequence Synthetic polynucleotide
  pITR-CMV-hPou4F3-T2A-mScarlet-DD
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatgatggccatgaactccaagcagcctttcggcatgcacccggtgctgcaagaacccaaattctccagtctgcactctggctccgaggccatgcgccgagtctgtctcccagccccgcagctgcagggtaatatatttggaagctttgatgagagcctgctggcacgcgccgaagctctggcggcggtggatatcgtctcccacggcaagaaccatccgttcaagcccgacgccacctaccataccatgagcagcgtgccctgcacgtccacttcgtccaccgtgcccatctcccacccagctgcgctcacctcacaccctcaccacgccgtgcaccagggcctcgaaggcgacctgctggagcacatctcgcccacgctgagtgtgagcggcctgggcgctccggaacactcggtgatgcccgcacagatccatccacaccacctgggcgccatgggccacctgcaccaggccatgggcatgagtcacccgcacaccgtggcccctcatagcgccatgcctgcatgcctcagcgacgtggagtcagacccgcgcgagctggaagccttcgccgagcgcttcaagcagcggcgcatcaagctgggggtgacccaggcggacgtgggcgcggctctggctaatctcaagatccccggcgtgggctcgctgagccaaagcaccatctgcaggttcgagtctctcactctctcgcacaacaacatgatcgctctcaagccggtgctccaggcctggttggaggaggccgaggccgcctaccgagagaagaacagcaagccagagctcttcaacggcagcgaacggaagcgcaaacgcacgtccatcgcggcgccggagaagcgttcactcgaggcctatttcgctatccagccacgtccttcatctgagaagatcgcggccatcgctgagaaactggaccttaaaaagaacgtggtgagagtctggttctgcaaccagagacagaaacagaaacgaatgaagtattcggctgtccacgttaacgattacaaggatgacgacgataaggactataaggacgatgatgacaaggactacaaagatgatgacgataaaggatccggcgagggcagaggaagtctgctaacatgcggtgacgtcgaggagaatcctggcccaatgcctaagaagaagcggaaagtcggctccggcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 61 - DNA, 1020 bp, Homo sapiens
atgatggccatgaactccaagcagcctttcggcatgcacccggtgctgcaagaacccaaattctccagtctgcactctggctccgaggccatgcgccgagtctgtctcccagccccgcagctgcagggtaatatatttggaagctttgatgagagcctgctggcacgcgccgaagctctggcggcggtggatatcgtctcccacggcaagaaccatccgttcaagcccgacgccacctaccataccatgagcagcgtgccctgcacgtccacttcgtccaccgtgcccatctcccacccagctgcgctcacctcacaccctcaccacgccgtgcaccagggcctcgaaggcgacctgctggagcacatctcgcccacgctgagtgtgagcggcctgggcgctccggaacactcggtgatgcccgcacagatccatccacaccacctgggcgccatgggccacctgcaccaggccatgggcatgagtcacccgcacaccgtggcccctcatagcgccatgcctgcatgcctcagcgacgtggagtcagacccgcgcgagctggaagccttcgccgagcgcttcaagcagcggcgcatcaagctgggggtgacccaggcggacgtgggcgcggctctggctaatctcaagatccccggcgtgggctcgctgagccaaagcaccatctgcaggttcgagtctctcactctctcgcacaacaacatgatcgctctcaagccggtgctccaggcctggttggaggaggccgaggccgcctaccgagagaagaacagcaagccagagctcttcaacggcagcgaacggaagcgcaaacgcacgtccatcgcggcgccggagaagcgttcactcgaggcctatttcgctatccagccacgtccttcatctgagaagatcgcggccatcgctgagaaactggaccttaaaaagaacgtggtgagagtctggttctgcaaccagagacagaaacagaaacgaatgaagtattcggctgtccacgttaac

SEQ ID NO: 62 - DNA, 81 bp, Artificial Sequence
  Description of Artificial Sequence Synthetic oligonucleotide
  3x FLAG
gattacaaggatgacgacgataaggactataaggacgatgatgacaaggactacaaagatgatgacgataaaggatccggc

SEQ ID NO: 63 - DNA, 54 bp, Artificial Sequence
  Description of Artificial Sequence Synthetic oligonucleotide
  T2A
gagggcagaggaagtctgctaacatgcggtgacgtcgaggagaatcctggccca

SEQ ID NO: 64 - DNA, 3692 bp, Artificial Sequence
  Description of Artificial Sequence Synthetic polynucleotide
  pITR-CMV-hGF11-T2A-mScarlet-DD
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatgccgcgctcatttctcgtcaaaagcaagaaggctcacagctaccaccagccgcgctccccaggaccagactattccctccgtttagagaatgtaccggcgcctagccgagcagacagcacttcaaatgcaggcggggcgaaggcggagccccgggaccgtttgtcccccgaatcgcagctgaccgaagccccagacagagcctccgcatccccagacagctgcgaaggcagcgtctgcgaacggagctcggagtttgaggacttctggaggcccccgtcaccctccgcgtctccagcctcggagaagtcaatgtgcccatcgctggacgaagcccagcccttccccctgcctttcaaaccgtactcatggagcggcctggcgggttctgacctgcggcacctggtgcagagctaccgaccgtgtggggccctggagcgtggcgctggcctgggcctcttctgcgaacccgccccggagcctggccacccggccgcgctgtacggcccgaagcgggctgccggcggcgcgggggccggggcgccagggagctgcagcgcaggggccggtgccaccgctggccctggcctagggctctacggcgacttcgggtctgcggcagccgggctgtatgagaggcccacggcagcggcgggcttgctgtaccccgagcgtggccacgggctgcacgcagacaagggcgctggcgtcaaggtggagtcggagctgctgtgcacccgcctgctgctgggcggcggctcctacaagtgcatcaagtgcagcaaggtgttctccacgccgcacgggctcgaggtgcacgtgcgcaggtcccacagcggcaccagaccctttgcctgcgagatgtgcggcaagaccttcgggcacgcggtgagcctggagcagcacaaagccgtgcactcgcaggaacggagctttgactgtaagatctgtgggaagagcttcaagaggtcatccacactgtccacacacctgcttatccactcagacactcggccctacccctgtcagtactgtggcaagaggttccaccagaagtcagacatgaagaaacacactttcatccacactggtgagaagcctcacaagtgccaggtgtgcggcaaggcattcagccagagctccaacctcatcacccacagccgcaaacacacaggcttcaagcccttcggctgcgacctctgtgggaagggtttccagaggaaggtggacctccgaaggcaccgggagacgcagcatgggctcaaagttaacgattacaaggatgacgacgataaggactataaggacgatgatgacaaggactacaaagatgatgacgataaaggatccggcgagggcagaggaagtctgctaacatgcggtgacgtcgaggagaatcctggcccaatgcctaagaagaagcggaaagtcggctccggcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 65 - DNA, 1272 bp, Homo sapiens
atgccgcgctcatttctcgtcaaaagcaagaaggctcacagctaccaccagccgcgctccccaggaccagactattccctccgtttagagaatgtaccggcgcctagccgagcagacagcacttcaaatgcaggcggggcgaaggcggagccccgggaccgtttgtcccccgaatcgcagctgaccgaagccccagacagagcctccgcatccccagacagctgcgaaggcagcgtctgcgaacggagctcggagtttgaggacttctggaggcccccgtcaccctccgcgtctccagcctcggagaagtcaatgtgcccatcgctggacgaagcccagcccttccccctgcctttcaaaccgtactcatggagcggcctggcgggttctgacctgcggcacctggtgcagagctaccgaccgtgtggggccctggagcgtggcgctggcctgggcctcttctgcgaacccgccccggagcctggccacccggccgcgctgtacggcccgaagcgggctgccggcggcgcgggggccggggcgccagggagctgcagcgcaggggccggtgccaccgctggccctggcctagggctctacggcgacttcgggtctgcggcagccgggctgtatgagaggcccacggcagcggcgggcttgctgtaccccgagcgtggccacgggctgcacgcagacaagggcgctggcgtcaaggtggagtcggagctgctgtgcacccgcctgctgctgggcggcggctcctacaagtgcatcaagtgcagcaaggtgttctccacgccgcacgggctcgaggtgcacgtgcgcaggtcccacagcggcaccagaccctttgcctgcgagatgtgcggcaagaccttcgggcacgcggtgagcctggagcagcacaaagccgtgcactcgcaggaacggagctttgactgtaagatctgtgggaagagcttcaagaggtcatccacactgtccacacacctgcttatccactcagacactcggccctacccctgtcagtactgtggcaagaggttccaccagaagtcagacatgaagaaacacactttcatccacactggtgagaagcctcacaagtgccaggtgtgcggcaaggcattcagccagagctccaacctcatcacccacagccgcaaacacacaggcttcaagcccttcggctgcgacctctgtgggaagggtttccagaggaaggtggacctccgaaggcaccgggagacgcagcatgggctcaaagttaac

SEQ ID NO: 66 - DNA, 3488 bp, Artificial Sequence
  Description of Artificial Sequence Synthetic polynucleotide
  pITR-CMV-hATOH1-T2A-mScarlet-DD
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagtgttaacgattacaaggatgacgacgataaggactataaggacgatgatgacaaggactacaaagatgatgacgataaaggatccggcgagggcagaggaagtctgctaacatgcggtgacgtcgaggagaatcctggcccaatgcctaagaagaagcggaaagtcggctccggcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 67 - DNA, 1068 bp, Homo sapiens
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagtgttaac

SEQ ID NO: 68 - DNA, 3690 bp, Artificial Sequence
  Description of Artificial Sequence Synthetic polynucleotide
  pITR-CMV-Luc2-T2A-mScarlet-U6-Hes1-S3
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacacaggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggctccggagagggcagaggaagtctgctaacatgcggtgacgtcgaggagaatcctggcccaatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaagctgatcagcctcgataagatacattgatgagtttggacaaaccacaactagaatgcagtgaaaaaaatgctttatttgtgaaatttgtgatgctattgctttatttgtaaccattataagctgcaataaacaagttaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaagtcatcaaagcctatcgaaataggctttgatgactttcttttttaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgc

SEQ ID NO: 69 - DNA, 1659 bp, Artificial Sequence
  Description of Artificial Sequence Synthetic polynucleotide
  luciferase sequence
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacacaggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggctccgga

SEQ ID NO: 70 - DNA, 137 bp, Artificial Sequence
  Description of Artificial Sequence Synthetic polynucleotide
  SV40pA
gctgatcagcctcgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 71 - DNA, 264 bp, Artificial Sequence
  Description of Artificial Sequence Synthetic polynucleotide
  U6
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 72 - DNA, 50 bp, Artificial Sequence
  Description of Artificial Sequence Synthetic oligonucleotide
  siRNA DNA sequence
  modified_base (1)..(20): a, c, g, or t
  modified_base (25)..(44): a, c, g, or t
nnnnnnnnnnnnnnnnnnnncgaannnnnnnnnnnnnnnnnnnntttttt

SEQ ID NO: 73 - DNA, 19 bp, Artificial Sequence
  Description of Artificial Sequence Synthetic oligonucleotide
  S3 shRNA
gaaagtcatcaaagcctat

SEQ ID NO: 74 - DNA, 19 bp, Artificial Sequence
  Description of Artificial Sequence Synthetic oligonucleotide
  S5 shRNA
actgcatgacccagatcaa

SEQ ID NO: 75 - DNA, 19 bp, Artificial Sequence
  Description of Artificial Sequence Synthetic oligonucleotide
  Kop shRNA
actgcatgacccagatcaa

SEQ ID NO: 76 - DNA, 1444 bp, Artificial Sequence
  Description of Artificial Sequence Synthetic polynucleotide
  pITR-CMV-mScarlet-bGHpA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 77 - DNA, 1918 bp, Artificial Sequence
  Description of Artificial Sequence Synthetic polynucleotide
  pITR-CMV-mScarlet-DD-bGHpA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaaatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 78 - DNA, 3690 bp, Artificial Sequence
  Description of Artificial Sequence Synthetic polynucleotide
  pITR-CMV-Luc2-T2A-GFP-U6-Hes1-S5
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacacaggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggctccggagagggcagaggaagtctgctaacatgcggtgacgtcgaggagaatcctggcccaatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaagctgatcagcctcgataagatacattgatgagtttggacaaaccacaactagaatgcagtgaaaaaaatgctttatttgtgaaatttgtgatgctattgctttatttgtaaccattataagctgcaataaacaagttaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccactgcatgacccagatcaacgaattgatctgggtcatgcagtttttttaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgc

SEQ ID NO: 79 - DNA, 3691 bp, Artificial Sequence
  Description of Artificial Sequence Synthetic polynucleotide
  pITR-CMV-Luc2-T2A-GFP-U6-Hes1-KOP
cctgcaggcagctgcgcgctcgctcgctcactgaggccgcccgggcgtcgggcgacctttggtcgcccggcctcagtgagcgagcgagcgcgcagagagggagtggccaactccatcactaggggttcctctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacacaggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggctccggagagggcagaggaagtctgctaacatgcggtgacgtcgaggagaatcctggcccaatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaagctgatcagcctcgataagatacattgatgagtttggacaaaccacaactagaatgcagtgaaaaaaatgctttatttgtgaaatttgtgatgctattgctttatttgtaaccattataagctgcaataaacaagttaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagctgatataatggagaacgaattctccattatatcagctgttttttaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgc

SEQ ID NO: 80 - DNA, 20 bp, Artificial Sequence
  Description of Artificial Sequence Synthetic oligonucleotide
  Hes1-KOP
gcagctgatataatggagaa

SEQ ID NO: 81 - DNA, 4221 bp, Artificial Sequence
  Description of Artificial Sequence Synthetic polynucleotide
  pITR-CMV-mScarlet
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcaccggtgccaccatgcctaagaagaagcggaaagtcggctccggcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 82 - DNA, 4701 bp, Artificial Sequence
  Description of Artificial Sequence Synthetic polynucleotide
  pITR-CMV-mScarlet-DD
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcaccggtgccaccatgcctaagaagaagcggaaagtcggctccggcgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 83 - DNA, 4218 bp, Artificial Sequence
  Description of Artificial Sequence Synthetic polynucleotide
  pITR-U6-shHES1-S5-CMV-3xFLAG-hATOH1-DD-T2A- hPOU4F3-U6-shHES1-S3
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcaaaaaaactgcatgacccagatcaattcgttgatctgggtcatgcagtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccacgcgtctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctaccggtgccaccatggattacaaggatgacgacgataaggactataaggacgatgatgacaaggactacaaagatgatgacgataaagttaactcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagtgttaacatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgaggatccggcgagggcagaggaagtctgctaacatgcggtgacgtcgaggagaatcctggcccaatgatggccatgaactccaagcagcctttcggcatgcacccggtgctgcaagaacccaaattctccagtctgcactctggctccgaggccatgcgccgagtctgtctcccagccccgcagctgcagggtaatatatttggaagctttgatgagagcctgctggcacgcgccgaagctctggcggcggtggatatcgtctcccacggcaagaaccatccgttcaagcccgacgccacctaccataccatgagcagcgtgccctgcacgtccacttcgtccaccgtgcccatctcccacccagctgcgctcacctcacaccctcaccacgccgtgcaccagggcctcgaaggcgacctgctggagcacatctcgcccacgctgagtgtgagcggcctgggcgctccggaacactcggtgatgcccgcacagatccatccacaccacctgggcgccatgggccacctgcaccaggccatgggcatgagtcacccgcacaccgtggcccctcatagcgccatgcctgcatgcctcagcgacgtggagtcagacccgcgcgagctggaagccttcgccgagcgcttcaagcagcggcgcatcaagctgggggtgacccaggcggacgtgggcgcggctctggctaatctcaagatccccggcgtgggctcgctgagccaaagcaccatctgcaggttcgagtctctcactctctcgcacaacaacatgatcgctctcaagccggtgctccaggcctggttggaggaggccgaggccgcctaccgagagaagaacagcaagccagagctcttcaacggcagcgaacggaagcgcaaacgcacgtccatcgcggcgccggagaagcgttcactcgaggcctatttcgctatccagccacgtccttcatctgagaagatcgcggccatcgctgagaaactggaccttaaaaagaacgtggtgagagtctggttctgcaaccagagacagaaacagaaacgaatgaagtattcggctgtccactaaataataaaatatctttattttcattacatctgtgtgttggttttttgtgtgttaattaaaaaaaagaaagtcatcaaagcctatttcgataggctttgatgactttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacccggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 84 - DNA, 263 bp, Artificial Sequence
  Description of Artificial Sequence Synthetic polynucleotide
  U6
cggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacc

SEQ ID NO: 85 - DNA, 263 bp, Artificial Sequence; identical to SEQ ID NO: 84
  Description of Artificial Sequence Synthetic polynucleotide
  shHES1-1

SEQ ID NO: 86 - DNA, 75 bp, Artificial Sequence
  Description of Artificial Sequence Synthetic oligonucleotide
  3x FLAG
atggattacaaggatgacgacgataaggactataaggacgatgatgacaaggactacaaagatgatgacgataaa

SEQ ID NO: 87 - DNA, 1065 bp, Homo sapiens
gttaactcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagt

SEQ ID NO: 88 - DNA, 474 bp, Artificial Sequence
  Description of Artificial Sequence Synthetic polynucleotide
  Destabilizing Domain
atcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcga

SEQ ID NO: 89 - DNA, 56 bp, Artificial Sequence
  Description of Artificial Sequence Synthetic oligonucleotide
  T2A
gcgagggcagaggaagtctgctaacatgcggtgacgtcgaggagaatcctggccca

SEQ ID NO: 90 - DNA, 51 bp, Artificial Sequence
  Description of Unknown Bovidae bGH PolyA sequence
ataataaaatatctttattttcattacatctgtgtgttggttttttgtgtg

SEQ ID NO: 91 - DNA, 262 bp, Artificial Sequence
  Description of Artificial Sequence Synthetic polynucleotide
  U6
ggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacc

SEQ ID NO: 92 - DNA, 262 bp, Artificial Sequence; identical to SEQ ID NO: 91
  Description of Artificial Sequence Synthetic polynucleotide
  shHES1-2

SEQ ID NO: 93 - DNA, 4930 bp, Artificial Sequence
  Description of Artificial Sequence Synthetic polynucleotide
  pITR-U6-shHES1-S5, hATOHessps-3xFLAG-hATOH1-T2A-hPOU4F3-US-shHES1-S3
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcaaaaaaactgcatgacccagatcaattcgttgatctgggtcatgcagtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccacgcgtctatggagtttgcataacaaacgtttggcagctcgctctcttacactccattaacaagctgtaacatatagctgcaggttgctataatctcattaatattttggaaacttgaatattgagtatttctgagtgctcattccccatatgccagccacttctgccatgctgactggttcctttctctccattattagcaattagcttcttaccttccaaagtcagatccaaggtatccaagatactagcaaaggaatcaactatgtgtgcaagttaagcatgcttaatatcacccaaacaaacaaagaggcagcatttcttaaagtaatgaagatagataaatcgggttagtcctttgcgacactgctggtgctttctagagttttatatattttaagcagcttgctttatattctgtctttgcctcccaccccaccagcacttttatttgtggagggttttggctcgccacactttgggaaacttatttgatttcacggagagctgaaggaagatcatttttggcaacagacaagtttaaacacgatttctatgggacattgctaactggggcccctaaggagaaaggggaaactgagcggagaatgggttaaatccttggaagcaggggagaggcaggggaggagagaagtcggaggagtataaagaaaaggacaggaaccaagaagcgtgggggtggtttgccgtaatgtgagtgtttcttaattagagaacggttgacaatagagggtctggcagaggctcctggccgcggtgcggagcgtctggagcggagcacgcgctgtcagctggtgagcgcactctcctttcaggcagctccccggggagctgtgcggccacatttaacaccatcatcacccctccccggcctcctcaacctcggcctcctcctcgtcgacagccttccttggcccccaccagcagagctcacagtagcgagcgtctctcgccgtctcccgcactcggccggggcctctctcctcccccagctgcgcagcgggagccgccactgcccactgcacctcccagcaaccagcccagcacgcaaagaagctgcgcaaagttaaagccaagcaatgccaaggggaggggaagctggaggcgggctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaacatatgaatctgacgaatttaaaagaaggaaaaaaaaaaaaaaacctgagcaggcttgggagtcctctgcacacaagaacttttctcggggtgtaaaaactctttgattggctgctcgcacgcgcctgcccgcgccctccattggctgagaagacacgcgaccggcgcgaggagggggttgggagaggagcggggggagactgagtggcgcgtgccgctttttaaaggggcgcagcgccttcagcaaccggagaagcatagttgcacgcgacctggtgtgtgatctccgagtgggtgggggagggtcgaggagggaaaaaaaaataagacgttgcagaagagacccggaaagggccttttttttggttgagctggtgtcccagtgctgcctccgatcctgagcctccgagcctttgcagtgcaaccggtgccaccatggattacaaggatgacgacgataaggactataaggacgatgatgacaaggactacaaagatgatgacgataaagttaacatgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagtgttaacgagggcagaggaagtctgctaacatgcggtgacgtcgaggagaatcctggcccaatgatggccatgaactccaagcagcctttcggcatgcacccggtgctgcaagaacccaaattctccagtctgcactctggctccgaggccatgcgccgagtctgtctcccagccccgcagctgcagggtaatatatttggaagctttgatgagagcctgctggcacgcgccgaagctctggcggcggtggatatcgtctcccacggcaagaaccatccgttcaagcccgacgccacctaccataccatgagcagcgtgccctgcacgtccacttcgtccaccgtgcccatctcccacccagctgcgctcacctcacaccctcaccacgccgtgcaccagggcctcgaaggcgacctgctggagcacatctcgcccacgctgagtgtgagcggcctgggcgctccggaacactcggtgatgcccgcacagatccatccacaccacctgggcgccatgggccacctgcaccaggccatgggcatgagtcacccgcacaccgtggcccctcatagcgccatgcctgcatgcctcagcgacgtggagtcagacccgcgcgagctggaagccttcgccgagcgcttcaagcagcggcgcatcaagctgggggtgacccaggcggacgtgggcgcggctctggctaatctcaagatccccggcgtgggctcgctgagccaaagcaccatctgcaggttcgagtctctcactctctcgcacaacaacatgatcgctctcaagccggtgctccaggcctggttggaggaggccgaggccgcctaccgagagaagaacagcaagccagagctcttcaacggcagcgaacggaagcgcaaacgcacgtccatcgcggcgccggagaagcgttcactcgaggcctatttcgctatccagccacgtccttcatctgagaagatcgcggccatcgctgagaaactggaccttaaaaagaacgtggtgagagtctggttctgcaaccagagacagaaacagaaacgaatgaagtattcggctgtccactaaataataaaatatctttattttcattacatctgtgtgttggttttttgtgtgttaattaaaaaaaagaaagtcatcaaagcctatttcgataggctttgatgactttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacccggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 94 - DNA, 1725 bp, Homo sapiens
ctatggagtttgcataacaaacgtttggcagctcgctctcttacactccattaacaagctgtaacatatagctgcaggttgctataatctcattaatattttggaaacttgaatattgagtatttctgagtgctcattccccatatgccagccacttctgccatgctgactggttcctttctctccattattagcaattagcttcttaccttccaaagtcagatccaaggtatccaagatactagcaaaggaatcaactatgtgtgcaagttaagcatgcttaatatcacccaaacaaacaaagaggcagcatttcttaaagtaatgaagatagataaatcgggttagtcctttgcgacactgctggtgctttctagagttttatatattttaagcagcttgctttatattctgtctttgcctcccaccccaccagcacttttatttgtggagggttttggctcgccacactttgggaaacttatttgatttcacggagagctgaaggaagatcatttttggcaacagacaagtttaaacacgatttctatgggacattgctaactggggcccctaaggagaaaggggaaactgagcggagaatgggttaaatccttggaagcaggggagaggcaggggaggagagaagtcggaggagtataaagaaaaggacaggaaccaagaagcgtgggggtggtttgccgtaatgtgagtgtttcttaattagagaacggttgacaatagagggtctggcagaggctcctggccgcggtgcggagcgtctggagcggagcacgcgctgtcagctggtgagcgcactctcctttcaggcagctccccggggagctgtgcggccacatttaacaccatcatcacccctccccggcctcctcaacctcggcctcctcctcgtcgacagccttccttggcccccaccagcagagctcacagtagcgagcgtctctcgccgtctcccgcactcggccggggcctctctcctcccccagctgcgcagcgggagccgccactgcccactgcacctcccagcaaccagcccagcacgcaaagaagctgcgcaaagttaaagccaagcaatgccaaggggaggggaagctggaggcgggctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaacatatgaatctgacgaatttaaaagaaggaaaaaaaaaaaaaaacctgagcaggcttgggagtcctctgcacacaagaacttttctcggggtgtaaaaactctttgattggctgctcgcacgcgcctgcccgcgccctccattggctgagaagacacgcgaccggcgcgaggagggggttgggagaggagcggggggagactgagtggcgcgtgccgctttttaaaggggcgcagcgccttcagcaaccggagaagcatagttgcacgcgacctggtgtgtgatctccgagtgggtgggggagggtcgaggagggaaaaaaaaataagacgttgcagaagagacccggaaagggccttttttttggttgagctggtgtcccagtgctgcctccgatcctgagcctccgagcctttgcagtgcaa

SEQ ID NO: 95 - DNA, 1017 bp, Homo sapiens
atgatggccatgaactccaagcagcctttcggcatgcacccggtgctgcaagaacccaaattctccagtctgcactctggctccgaggccatgcgccgagtctgtctcccagccccgcagctgcagggtaatatatttggaagctttgatgagagcctgctggcacgcgccgaagctctggcggcggtggatatcgtctcccacggcaagaaccatccgttcaagcccgacgccacctaccataccatgagcagcgtgccctgcacgtccacttcgtccaccgtgcccatctcccacccagctgcgctcacctcacaccctcaccacgccgtgcaccagggcctcgaaggcgacctgctggagcacatctcgcccacgctgagtgtgagcggcctgggcgctccggaacactcggtgatgcccgcacagatccatccacaccacctgggcgccatgggccacctgcaccaggccatgggcatgagtcacccgcacaccgtggcccctcatagcgccatgcctgcatgcctcagcgacgtggagtcagacccgcgcgagctggaagccttcgccgagcgcttcaagcagcggcgcatcaagctgggggtgacccaggcggacgtgggcgcggctctggctaatctcaagatccccggcgtgggctcgctgagccaaagcaccatctgcaggttcgagtctctcactctctcgcacaacaacatgatcgctctcaagccggtgctccaggcctggttggaggaggccgaggccgcctaccgagagaagaacagcaagccagagctcttcaacggcagcgaacggaagcgcaaacgcacgtccatcgcggcgccggagaagcgttcactcgaggcctatttcgctatccagccacgtccttcatctgagaagatcgcggccatcgctgagaaactggaccttaaaaagaacgtggtgagagtctggttctgcaaccagagacagaaacagaaacgaatgaagtattcggctgtccactaa

SEQ ID NO: 96 - DNA, 508 bp, Artificial Sequence
  Description of Artificial Sequence Synthetic polynucleotide
  CMV enhancer-promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 97 - RNA, 5000 nt, Artificial Sequence
  Description of Artificial Sequence Synthetic polynucleotide
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 98 - DNA, 17 bp, Artificial Sequence
  Description of Unknown soluble neuropilin-1 (sNRP) sequence
aaataaaatacgaaatg

(19) United States
(12) Patent Application Publication
(10) Pub. No.: US 2022/0395582 A1
(43) Pub. Date: Dec. 15, 2022

(54) COMPOSITIONS AND METHODS OF INDUCING DIFFERENTIATION OF A HAIR CELL

(71) Applicant: Akouos, Inc., Boston, MA (US)

(72) Inventors: Emmanuel John Simons, Brookline, MA (US); Robert Ng, Newton, MA (US); Danielle R. Lenz, Brookline, MA (US); Michelle Valero, Revere, MA (US)

(21) Appl. No.: 17/291,906

(22) PCT Filed: Nov. 7, 2019
(86) PCT No.: PCT/US19/60324

§ 371 Date: May 6, 2021

Related U.S. Application Data
(60) Provisional application No. 62/756,910, filed on Nov. 7, 2018.
(60) Provisional application No. 62/888,105, filed on Aug. 16, 2019.

(51) Int. Cl. A61K 48/00; C12N 15/86; A61K 38/18; A61K 38/17; A61K 9/00; A61P 27/16

(52) U.S. Cl. CPC A61K 48/005; C12N 15/86; A61K 48/0075; A61K 38/185; A61K 38/1709; A61K 38/1841; A61K 38/177; A61K 9/0019; A61P 27/16; C12N 2750/14123; C12N 2750/14142

(57) ABSTRACT

Provided herein are compositions that include one or more adeno-associated virus (AAVs) vectors and methods of inducing differentiation of a hair cell using these vector(s).

### CROSS-REFERENCE TO RELATED APPLICATIONS

This application claims priority to U.S. Provisional Patent Application Ser. No. 62/756,910, filed Nov. 7, 2018 and U.S. Provisional Patent Application Ser. No. 62/888,105, filed Aug. 16, 2019; the entire contents of which are herein incorporated by reference.

### TECHNICAL FIELD

The present disclosure relates to the fields of molecular biology, and more specifically, to the use of nucleic acids for treating hearing loss in a primate.

### BACKGROUND OF THE INVENTION

Hearing loss can be conductive (arising from the ear canal or middle ear), sensorineural (arising from the inner ear or auditory nerve), or mixed. Most forms of non-syndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

### SUMMARY

The present invention is based on the discovery that administration of an AAV vector that includes a nucleic acid encoding a gene, to the inner ear of a primate, can result in the successful expression of a protein encoded by the gene in a supporting cell or hair cell in the inner ear of the primate. In view of this discovery, provided here are AAV vector(s) and methods of using these vectors to induce expression and/or activity of a hair cell differentiation protein in a supporting cell or hair cell in the inner ear of a primate or decreasing the expression and/or activity of a hair cell differentiation suppressing gene in a supporting cell or hair cell in the inner ear of a primate.

Provided herein are compositions that include at least two different nucleic acid vectors, where: each of the at least two different adeno-associated virus (AAV) vectors includes a coding sequence that encodes a different portion of a hair cell differentiation protein, each of the encoded portions being at least 30 amino acid residues in length, where the amino acid sequence of each of the encoded portions may optionally partially overlap with the amino acid sequence of a different one of the encoded portions; no single vector of the at least two different vectors encodes the full-length hair cell differentiation protein; at least one of the coding sequences includes a nucleotide sequence spanning two neighboring exons of hair cell differentiation genomic DNA, and lacks an intronic sequence between the two neighboring exons; and when introduced into a primate cell the at least two different vectors undergo concatamerization or homologous recombination with each other, thereby forming a recombined nucleic acid that encodes a full-length hair cell differentiation protein that is expressed in the primate cell.

In some embodiments of any of the compositions described herein, the amino acid sequence of none of the encoded portions overlaps with the amino acid sequence of a different one of the encoded portions. In some embodiments of any of the compositions described herein, the amino acid sequence of each of the encoded portions partially overlaps with the amino acid sequence of a different one of the encoded portions. In some embodiments of any of the compositions described herein, the overlapping amino acid sequence is between 30 amino acid residues to about 390 amino acid residues in length.

In some embodiments of any of the compositions described herein, the vectors include two different vectors, each of which includes a different segment of an intron, where the intron includes the nucleotide sequence of an intron that is present in a hair cell differentiation genomic DNA, and where the two different segments overlap in sequence by at least 100 nucleotides. In some embodiments of any of the compositions described herein, the two different intron segments overlap in sequence by about 100 nucleotides to about 800 nucleotides.

In some embodiments of any of the compositions described herein, the entire nucleotide sequence of each of the at least two different vectors is between about 500 nucleotides to about 10,000 nucleotides in length. In some embodiments of any of the compositions described herein, the entire nucleotide sequence of each of the at least two different vectors is between about 500 nucleotides to about 5,000 nucleotides in length.

In some embodiments of any of the compositions described herein, the number of different vectors in the composition is two. In some embodiments of any of the compositions described herein, a first of the two different vectors includes a coding sequence that encodes an N-terminal portion of the hair cell differentiation protein. In some embodiments of any of the compositions described herein, the N-terminal portion of the hair cell differentiation protein is between about 30 amino acids to about 750 amino acids in length. In some embodiments of any of the compositions described herein, the N-terminal portion of the hair cell differentiation protein is between about 30 amino acids to about 320 amino acids in length.

In some embodiments of any of the compositions described herein, the first vector further includes one or both of a promoter and a Kozak sequence. In some embodiments of any of the compositions described herein, the first vector includes a promoter that is an inducible promoter, a constitutive promoter, or a tissue-specific promoter.

In some embodiments of any of the compositions described herein, the second of the two different vectors includes a coding sequence that encodes a C-terminal portion of the hair cell differentiation protein. In some embodiments of any of the compositions described herein, the C-terminal portion of the hair cell differentiation protein is between about 30 amino acids to about 750 amino acids in length. In some embodiments of any of the compositions described herein, the C-terminal portion of the hair cell differentiation portion is between about 30 amino acids to about 320 amino acids in length.

In some embodiments of any of the compositions described herein, the second vector further includes a poly(dA) sequence. In some embodiments of any of the compositions described herein, the second vector further includes a destabilizing sequence. In some embodiments of any of the compositions described herein, the second vector further includes a FKB12 destabilizing sequence.

Also provided herein are compositions that include two different nucleic acid vectors, where: a first nucleic acid vector of the two different nucleic acid vectors includes a promoter, a first coding sequence that encodes an N-terminal portion of a hair cell differentiation protein positioned 3′ of the promoter, and a splicing donor signal sequence positioned at the 3′ end of the first coding sequence; and a second nucleic acid vector of the two different nucleic acid vectors includes a splicing acceptor signal sequence, a second coding sequence that encodes a C-terminal portion of a hair cell differentiation protein positioned at the 3′ end of the splicing acceptor signal sequence, and a polyadenylation sequence at the 3′ end of the second coding sequence; where each of the encoded portions is at least 30 amino acid residues in length, where the amino acid sequences of the encoded portions do not overlap, where no single vector of the two different vectors encodes the full-length hair cell differentiation protein, and, when the coding sequences are transcribed in a primate cell, to produce RNA transcripts, splicing occurs between the splicing donor signal sequence on one transcript and the splicing acceptor signal sequence on the other transcript, thereby forming a recombined RNA molecule that encodes a full-length hair cell differentiation protein.

In some embodiments of any of the compositions described herein, at least one of the coding sequences includes a nucleotide sequence spanning two neighboring exons of a hair cell differentiation genomic DNA, and lacks an intronic sequence between the two neighboring exons.

Also provided herein are compositions that include: a first nucleic acid vector including a promoter, a first coding sequence that encodes an N-terminal portion of a hair cell differentiation protein positioned 3′ of the promoter, a splicing donor signal sequence positioned at the 3′ end of the first coding sequence, and a first detectable marker gene positioned 3′ of the splicing donor signal sequence; and a second nucleic acid vector, different from the first nucleic acid vector, including a second detectable marker gene, a splicing acceptor signal sequence positioned 3′ of the second detectable marker gene, a second coding sequence that encodes a C-terminal portion of a hair cell differentiation protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell differentiation protein, and, when the coding sequences are transcribed in a primate cell to produce RNA transcripts, splicing occurs between the splicing donor signal on one transcript and the splicing acceptor signal on the other transcript, thereby forming a recombined RNA molecule that encodes a full-length hair cell differentiation protein.

In some embodiments of any of the compositions described herein, at least one of the coding sequences includes a nucleotide sequence spanning two neighboring exons of a hair cell differentiation genomic DNA, and lacks an intronic sequence between the neighboring exons. In some embodiments of any of the compositions described herein, the first or second detectable marker gene is alkaline phosphatase. In some embodiments of any of the compositions described herein, the first and second detectable marker genes are the same.

Also provided herein are compositions that include: a first nucleic acid vector including a promoter, a first coding sequence that encodes an N-terminal portion of a hair cell differentiation protein positioned 3′ to the promoter, a splicing donor signal sequence positioned at the 3′ end of the first coding sequence, and a F1 phage recombinogenic region positioned 3′ to the splicing donor signal sequence; and a second nucleic acid vector, different from the first nucleic acid vector, including a second F1 phage recombinogenic region, a splicing acceptor signal sequence positioned 3′ of the second F1 phage recombinogenic region, a second coding sequence that encodes a C-terminal portion of a hair cell differentiation protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell differentiation protein, and, when the coding sequences are transcribed in a primate cell to produce RNA transcripts, splicing occurs between the splicing donor signal one transcript and the splicing acceptor signal on the other transcript, thereby forming a recombined RNA molecule that encodes a full-length hair cell differentiation protein.

In some embodiments of any of the compositions described herein, at least one of the coding sequences includes a nucleotide sequence spanning two neighboring exons of a hair cell differentiation genomic DNA, and lacks an intronic sequence between the two neighboring exons.

Also provided herein are compositions that include a single adeno-associated virus (AAV) vector, where the single AAV vector includes a nucleic acid sequence that encodes a hair cell differentiation protein; and when introduced into a mammalian cell (e.g., primate cell (e.g., a hair cell or a supporting cell of the inner ear), a nucleic acid encoding the hair cell differentiation protein is generated at the locus of the hair cell differentiation gene and the primate cell expresses the hair cell differentiation protein.

In some embodiments of any of the compositions described herein, the hair cell differentiation gene is selected from the group of: atonal bHLH transcription factor 1 (ATOH1), POU Class 4 Homeobox 3 (POU4F3), catenin beta 1 (CTNNB1), Noggin (NOG), growth factor independent 1 transcriptional repressor (GFI-1), neurotrophin 3 (NTF3), and brain-derived neurotrophic factor (BDNF).

Also provided herein are compositions including two different nucleic acid vectors, wherein a first nucleic acid vector includes a first nucleic acid sequence that encodes a first hair cell differentiation protein (e.g., any of the hair cell differentiation proteins described herein); and a second nucleic acid vector includes a second nucleic acid sequence that encodes a second hair cell differentiation protein (e.g., any of the hair cell differentiation proteins described herein), and when introduced into a primate cell, the first nucleic acid and the second nucleic acid encoding the first hair cell differentiation protein and the second hair cell differentiation protein are generated at the locus of the hair cell differentiation gene and the primate cell expresses the first hair cell differentiation protein and the second hair cell differentiation protein.

In some embodiments, the first and the second hair cell differentiation proteins are selected from the group consisting of: atonal bHLH transcription factor 1 (ATOH1), POU Class 4 Homeobox 3 (POU4F3), catenin beta 1 (CTNNB1), Noggin (NOG), growth factor independent 1 transcriptional repressor (GFI-1), neurotrophin 3 (NTF3) and brain-derived neurotrophic factor (BDNF).

In some embodiments of any of the compositions described herein, the second nucleic acid vector further includes a destabilizing sequence.

In some embodiments, the second nucleic acid vector further includes a FKB12 destabilizing sequence.

Provided herein are compositions that include at least one adeno-associated virus (AAV) vector that encodes an inhibitory nucleic acid that decreases the expression of a hair cell differentiation-suppressing protein in a primate cell.

In some embodiments of any of the compositions described herein, the inhibitory nucleic acid is a short interfering RNA (siRNA), a short hairpin RNA (shRNA), an antisense oligonucleotide, or a ribozyme.

In some embodiments of any of the compositions described herein, the hair cell differentiation-suppressing gene is 1-IES1, HES5, sex determining region Y-box 2 (SOX2), and p27kip (CDKN1B). In some embodiments of any of the compositions described herein, the composition further includes a pharmaceutically acceptable excipient. Also provided herein are kits including any of the compositions described herein. In some embodiments of any of the kits described herein, the kit further includes a pre-loaded syringe containing the composition.

Also provided herein are methods of promoting differentiation of a supporting cell of an inner ear of a primate into a hair cell that include: administering to the inner ear of the primate a therapeutically effective amount of any of the compositions described herein, where the administering promotes differentiation of the supporting cell of the inner ear of the primate into a hair cell.

Also provided herein are methods of increasing the expression level of a hair cell differentiation protein in a supporting cell or hair cell of an inner ear of a primate that include: administering to the inner ear of the primate a therapeutically effective amount of any of the compositions described herein, where the administering results in an increase in the expression level of the hair cell differentiation protein in the supporting cell or hair cell of the inner ear of the primate. In some embodiments of any of the methods described herein, the hair cell differentiation protein is selected from the group of: Atoh1, Pou4f3, β-Catenin, Noggin, GFI-1, NTF3, and BDNF. In some embodiments of the methods described herein, the primate has previously been determined to have a defective hair cell differentiation gene.

Also provided herein are methods of decreasing the expression level of a hair cell differentiation-suppressing protein in a supporting cell or hair cell of an inner ear of a primate that include: administering to the inner ear of the primate a therapeutically effective amount of any of the compositions described herein, where the administering results in a decrease in the expression level of the hair cell differentiation-suppressing protein in the supporting cell or hair cell of the inner ear of the primate.

Also provided herein are methods of increasing the number of functional hair cells in a primate in need thereof that include: administering to the inner ear of the primate a therapeutically effective amount of any of the compositions described herein.

Also provided herein are methods of improving hearing in a primate in need thereof that include: administering to the inner ear of the primate a therapeutically effective amount of any of the compositions described herein.

In some embodiments of any of the methods described herein, the method further includes prior to the administering step, determining that the primate has a defective hair cell differentiation gene.

Also provided herein are methods of repairing a hair cell toxicity-inducing mutation in an endogenous hair cell differentiation gene locus in a supporting cell or hair cell of an inner ear of a primate, that include: administering to the inner ear of the primate a therapeutically effective amount of any of the compositions described herein, where the administering results in repair of the hair cell toxicity-inducing mutation in the endogenous hair cell differentiation gene locus in the supporting cell or hair cell of the inner ear of the primate.

Also provided herein are methods of decreasing the risk of hearing loss due to hair cell loss or dysfunction in a primate in need thereof that include: administering to the inner ear of the primate a therapeutically effective amount of any of the compositions described herein.

In some embodiments of any of the methods described herein, the primate has been previously identified as having a defective hair cell differentiation gene.

The term “a” and “an” refers to one or to more than one (i.e., at least one) of the grammatical object of the article.

The term “conservative mutation” refers to a mutation that does not change the amino acid encoded at the site of the mutation (due to codon degeneracy).

Modifications can be introduced into a nucleotide sequence by standard techniques known in the art, such as site-directed mutagenesis and PCR-mediated mutagenesis.

Conservative amino acid substitutions are ones in which the amino acid residue in a protein is replaced with an amino acid residue having a chemically-similar side chain. Families of amino acid residues having similar side chains have been defined in the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, cysteine, and tryptophan), nonpolar side chains (e.g., alanine, valine, leucine, isoleucine, proline, phenylalanine, and methionine), beta-branched side chains (e.g., threonine, valine, and isoleucine), and aromatic side chains (e.g., tyrosine, phenylalanine, tryptophan, and histidine).

Unless otherwise specified, a “nucleotide sequence encoding an amino acid sequence” includes all nucleotide sequences that are degenerate versions of each other and thus encode the same amino acid sequence.

The term “endogenous” refers to any material originating from within an organism, cell, or tissue.

The term “exogenous” refers to any material introduced from or originating from outside an organism, cell, or tissue that is not produced or does not originate from the same organism, cell, or tissue in which it is being introduced.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “transfected,” “transformed,” or “transduced” refers to a process by which exogenous nucleic acid is transferred or introduced into a cell. A “transfected,” “transformed,” or “transduced” primate cell is one that has been transfected, transformed, or transduced with exogenous nucleic acid.

The term “expression” refers to the transcription and/or translation of a particular nucleotide sequence encoding a protein.

The term “transient expression” refers to the expression of a non-integrated coding sequence for a short period of time (e.g., hours or days). The coding sequence that is transiently expressed in a cell (e.g., a primate cell) is lost upon multiple rounds of cell division.

The term “primate” is intended to include any primate (e.g., a human, a non-human primate (e.g., simian (e.g., a monkey (e.g., a marmoset, a baboon, a macaque), or an ape (e.g., a gorilla, a gibbon, an orangutan, or a chimpanzee). In some embodiments, the primate has or is at risk of having hearing loss. In some embodiments, the primate has been previously identified as having a mutation in a hair cell differentiation gene and/or a hair cell differentiation-suppressing gene. In some embodiments, the primate has been previously identified as having a mutation in a hair cell differentiation gene. In some embodiments, the primate has been previously identified as having a mutation in a hair cell differentiation-suppressing gene. In some embodiments, the primate has been identified as having a mutation in hair cell differentiation gene and/or a hair cell differentiation-suppressing gene and has been diagnosed with hearing loss. In some embodiments, the primate has been identified as having hearing loss.

A treatment is “therapeutically effective” when it results in a reduction in one or more of the number, severity, and frequency of one or more symptoms of a disease state (e.g., non-syndromic sensorineural hearing loss or syndromic sensorineural hearing loss) in a primate. In some embodiments, a therapeutically effective amount of a composition can result in an increase in the expression level of an active hair cell differentiation protein (e.g., a wildtype, full-length hair cell differentiation protein, or an active variant of a hair cell differentiation protein) (e.g., as compared to the expression level prior to treatment with the composition). In some embodiments, a therapeutically effective amount of a composition can result in an increase in the expression level of an active hair cell differentiation protein (e.g., a wildtype, full-length hair cell differentiation protein or active variant) in a target cell (e.g., a supporting cell of the inner ear or a hair cell (e.g., an outer hair cell or an inner hair cell) of the inner ear). In some embodiments, a therapeutically effective amount of a composition can result in an increase in the expression level of an active hair cell differentiation protein (e.g., a wildtype, full-length hair cell differentiation protein or active variant), and/or an increase in one or more activities of a hair cell differentiation protein in a target cell (e.g., as compared to a reference level, such as the level(s) in a primate cell prior to treatment, the level(s) in a primate cell having a mutation in a hair cell differentiation gene, or the level(s) in a primate cell or a population of primate cells from a subject having non-syndromic sensorineural hearing loss, or the level(s) in a primate cell or a population of primate cells from a subject having syndromic sensorineural hearing loss).

The term “nucleic acid” or “polynucleotide” refers to deoxyribonucleic acid (DNA) or ribonucleic acid (RNA), or a combination thereof, in either single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

The term “hair cell toxicity-inducing mutation” refers to a mutation in a hair cell differentiation gene that encodes a protein that when expressed (e.g., by a supporting cell or a hair cell) induces toxicity in a hair cell (e.g., in a primate).

The term “active hair cell differentiation protein” means a protein encoded by DNA that, if substituted for both wildtype alleles encoding full-length hair cell differentiation protein in supporting cells of the inner ear of what is otherwise a wildtype primate, and if expressed in the supporting cells of that primate, results in that primate's having a level of hearing approximating the normal level of hearing of a similar primate that is entirely wildtype. Non-limiting examples of active hair cell differentiation proteins are full-length hair cell differentiation proteins (e.g., any of the full-length hair cell differentiation proteins described herein).

The term “inhibitory nucleic acid” refers to a nucleic acid sequence that hybridizes specifically to a target gene or a target mRNA (e.g., a hair cell differentiation-suppressing gene or a hair cell differentiation-suppressing mRNA) and thereby inhibits the expression and/or activity of the target gene or the target mRNA (e.g., a hair cell differentiation-suppressing gene or a hair cell differentiation-suppressing mRNA). In some embodiments, the inhibitory nucleic acid is a short interfering RNA (siRNA), a short hairpin RNA (shRNA), an antisense oligonucleotide, or a ribozyme. In some embodiments, the inhibitory nucleic acid is between about 10 nucleotides to about 30 nucleotides in length (e.g., about 10 nucleotides to about 28 nucleotides, about 10 nucleotides to about 26 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 30 nucleotides, about 12 nucleotides to about 28 nucleotides, about 12 nucleotides to about 26 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 16 nucleotides to about 30 nucleotides, about 16 nucleotides to about 28 nucleotides, about 16 nucleotides to about 26 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 30 nucleotides, about 18 nucleotides to about 28 nucleotides, about 18 nucleotides to about 26 nucleotides, about 18 nucleotides to about 24 nucleotides, about 18 nucleotides to about 22 nucleotides, about 18 nucleotides to about 20 nucleotides, about 20 nucleotides to about 30 nucleotides, about 20 nucleotides to about 28 nucleotides, about 20 nucleotides to about 26 nucleotides, about 20 nucleotides to about 24 nucleotides, about 20 nucleotides to about 22 nucleotides, about 22 nucleotides to about 30 nucleotides, about 22 nucleotides to about 28 nucleotides, about 22 nucleotides to about 26 nucleotides, about 22 nucleotides to about 24 nucleotides, about 24 nucleotides to about 30 nucleotides, about 24 nucleotides to about 28 nucleotides, about 24 nucleotides to about 26 nucleotides, about 26 nucleotides to about 30 nucleotides, about 26 nucleotides to about 28 nucleotides, about 28 nucleotides to about 30 nucleotides, or 11, 12, 13, 14, 15, 16, 17, 18, 19, 20, 21, 22, 23, 24, 25, 26, 27, 28, 29 or 30 nucleotides).

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF DRAWINGS

FIG. 1A is a representative image of Myo7a/Iba-1 immunofluorescent staining of cochlear tissue of a cynomolgus macaque (non-human primate) following administration of a single Anc80-GFP AAV vector directly into the inner ear through the round window.

FIG. 1B is a representative image of Anc80-GFP immunofluorescent staining of the same cochlear tissue of the cynomolgus macaque as in FIG. 1A.

FIG. 1C is a representative image of a merged immunofluorescent staining of Myo7a/Iba-1 and Anc80-GFP of the same cochlear tissue of the cynomolgus macaque as in FIG. 1A.

FIG. 2A is a representative image of Anc80-GFP immunofluorescent staining of a NHP cochlear tissue showing the stria vascularis, the spiral ligament and the lateral wall.

FIG. 2B is a representative image of Anc80-GFP immunofluorescent staining of the same NHP cochlear tissue as in FIG. 2A showing the spiral limbus, the inner sulcus, inner hair cells (IHC) and outer hair cells (OHC).

FIG. 3 is a simplified schematic diagram showing the proteins that play a role during the development of supporting cells and hair cells in the cochlea.

FIG. 4A is an exemplary nucleic acid vector (SEQ ID NO: 66), that includes an ITR sequence (SEQ ID NO: 51), a CMV enhancer sequence (SEQ ID NO: 52), a CMV promoter sequence (SEQ ID NO: 53), a human ATOH1 gene sequence (SEQ ID NO: 67), a 3×Flag sequence (SEQ ID NO: 62), a T2A sequence (SEQ ID NO: 63), a SV40-NLS sequence (SEQ ID NO: 54), a mScarlet gene sequence (SEQ ID NO: 55), a destabilizing domain (DD) sequence (SEQ ID NO: 59), a bGHpA sequence (SEQ ID NO: 56), and an ITR sequence (SEQ ID NO: 57).

FIG. 4B is an exemplary nucleic acid vector (SEQ ID NO: 64), that includes an ITR sequence (SEQ ID NO: 51), a CMV enhancer sequence (SEQ ID NO: 52), a CMV promoter sequence (SEQ ID NO: 53), a human GFI1 gene sequence (SEQ ID NO: 65), a 3×Flag sequence (SEQ ID NO: 62), a T2A sequence (SEQ ID NO: 63), a SV40-NLS sequence (SEQ ID NO: 54), a mScarlet sequence (SEQ ID NO: 55), a destabilizing domain (DD) sequence (SEQ ID NO: 59), a bGHpA sequence (SEQ ID NO: 56), and an ITR sequence (SEQ ID NO: 57).

FIG. 4C is an exemplary nucleic acid vector (SEQ ID NO: 60), that includes an ITR sequence (SEQ ID NO: 51), a CMV enhancer sequence (SEQ ID NO: 52), a CMV promoter sequence (SEQ ID NO: 53), a human POU4F3 gene sequence (SEQ ID NO: 61), a 3×Flag sequence (SEQ ID NO: 62), a T2A sequence (SEQ ID NO: 63), a SV40-NLS sequence (SEQ ID NO: 54), a mScarlet sequence (SEQ ID NO: 55), a destabilizing domain (DD) sequence (SEQ ID NO: 59), a bGHpA sequence (SEQ ID NO: 56), and an ITR sequence (SEQ ID NO: 57).

FIG. 4D is an exemplary nucleic acid vector (SEQ ID NO: 68), that includes an ITR sequence (SEQ ID NO: 51), a CMV enhancer sequence (SEQ ID NO: 52), a CMV promoter sequence (SEQ ID NO: 53), a luciferase (Fluc) gene sequence (SEQ ID NO: 69), a T2A sequence (SEQ ID NO: 63), an mScarlet gene sequence (SEQ ID NO: 55), a SV40 pA sequence (SEQ ID NO: 70), a U6 sequence (SEQ ID NO: 71), a short hairpin RNA (shRNA) sequence (SEQ ID NO: 72), and an ITR sequence (SEQ ID NO: 57).

FIG. 5A is bar graph showing the relative quantification of Hes-1 RNA in HEK293FT cells transfected with combinations of dual and triple shRNA constructs (S3 (GAAAGTCATCAAAGCCTAT; SEQ ID NO: 73), S5 (ACTGCATGACCCAGATCAA; SEQ ID NO: 74), Kop (ACTGCATGACCCAGATCAA; SEQ ID NO: 75), S3 plus S5, S3 plus Kop, and S5 plus Kop) as determined by real time quantitative polymerase chain reaction (RTqPCR).

FIG. 5B is bar graph showing the relative quantification of Hes-1 protein in HEK293FT cells transfected with combinations of dual and triple shRNA constructs (S3, S5, Kop, S3 plus S5, S3 plus Kop, and S5 plus Kop) as determined by Western blotting.

FIG. 6A is a bar graph showing the relative quantification of ATOH1, POU4F3, and GFI1 (APG) RN in HEK293FT cells transfected with the individual plasmids of FIGS. 4A-C.

FIG. 6B is an image of a Western blot showing the relative quantification of ATOH1, POU4F3 and GFI1 protein expression in HEK293FT cells transfected with the individual plasmids of FIGS. 4A-C.

FIG. 7A is an exemplary nucleic acid vector (SEQ ID NO: 76), that includes an ITR sequence (SEQ ID NO: 51), a CMV promoter sequence (SEQ ID NO: 53), a mScarlet sequence (SEQ ID NO: 55), a bGHpA sequence (SEQ ID NO: 56) and an ITR sequence (SEQ ID NO: 57).

FIG. 7B is an exemplary nucleic acid vector (SEQ ID NO: 77), that includes an ITR sequence (SEQ ID NO: 51), a CMV promoter sequence (SEQ ID NO: 53), a mScarlet sequence (SEQ ID NO: 55), a destabilizing domain (DD) sequence (SEQ ID NO: 59), a bGHpA sequence (SEQ ID NO: 56) and an ITR sequence (SEQ ID NO: 57).

FIG. 8A is a dose response curve showing the functionality and reversibility of the destabilizing domain (DD) using fluorescence microscopy. Serial dilutions of TMP (0.1 μM, 1 μM, 10 μM, 20 μM and 100 μM) were tested in the mScarlet and mScarlet-DD transfected HEK293FT cells.

FIG. 8B is a graph showing the functionality and reversibility of the destabilizing domain (DD) by flow cytometry (Attune flow cytometer).

FIG. 9A is an image showing mScarlet positive cells in a P1-P3 mouse cochlea explant transfected with AAVanc80 vector at various MOIs. 10 μM TMP was added at a later time point.

FIG. 9B is an image showing mScarlet positive HEK293FT cells transfected with AAVanc80 vector at various MOIs. 10 μM TMP was added at a later time point.

FIG. 10 is an image showing mScarlet positive hair cells and supporting cells in a cochlear explants infected with AAVanc80 with and without 10 uM TMP that was added at a later time point.

FIG. 11A is an exemplary nucleic acid vector (SEQ ID NO: 83), that includes an ITR sequence (SEQ ID NO: 51), a U6 sequence (SEQ ID NO: 84), a short hairpin HES1 RNA (shHES1) sequence (SEQ ID NO: 85), a CMV enhancer sequence (SEQ ID NO: 52), a CMV promoter sequence (SEQ ID NO: 53), a 3×Flag sequence (SEQ ID NO: 86), a human ATOH1 gene sequence (SEQ ID NO: 87), a destabilizing domain (DD) sequence (SEQ ID NO: 88), a T2A sequence (SEQ ID NO: 89), a human POU4F3 gene sequence (SEQ ID NO: 61), a bGHpA sequence (SEQ ID NO: 90), a U6 sequence (SEQ ID NO: 91), a short hairpin HES1 RNA (shHES1-2) sequence (SEQ ID NO: 92) and an ITR sequence (SEQ ID NO: 57).

FIG. 11B is an exemplary nucleic acid vector (SEQ ID NO: 93), that includes an ITR sequence (SEQ ID NO: 51), a U6 sequence (SEQ ID NO: 84), a short hairpin HES1 RNA (shHES-1) sequence (SEQ ID NO: 85), an ATOH1 enhancer-promoter sequence (SEQ ID NO: 94), a 3×Flag sequence (SEQ ID NO: 86), a human ATOH1 gene sequence (SEQ ID NO: 67), a T2A sequence (SEQ ID NO: 63), a human POU4F3 gene sequence (SEQ ID NO: 95), a bGHpA sequence (SEQ ID NO: 90), a U6 sequence (SEQ ID NO: 84), a short hairpin HES1 RNA (shHES1-2) sequence (SEQ ID NO: 92) and an ITR sequence (SEQ ID NO: 57).

FIG. 12A is a bar graph showing the relative quantification of ATOH1, POU4F3, and HES1 in HEK293FT cells transfected with the combined plasmids of FIGS. 11A-B.

FIG. 12B is an image of a Western blot showing the relative quantification of 3×Flag-ATOH1 and HES1 protein expression in HEK293FT cells transfected with the combined plasmids of FIGS. 11A-B.

### DETAILED DESCRIPTION

Provided herein are compositions including at least two different nucleic acid vectors, where: each of the at least two different adeno-associated virus (AAV) vectors comprises a coding sequence that encodes a different portion of a hair cell differentiation protein, each of the encoded portions being at least 30 amino acid residues in length, where the amino acid sequence of each of the encoded portions may optionally partially overlap with the amino acid sequence of a different one of the encoded portions; no single vector of the at least two different vectors encodes the full-length hair cell differentiation protein; at least one of the coding sequences includes a nucleotide sequence spanning two neighboring exons of hair cell differentiation genomic DNA, and lacks an intronic sequence between the two neighboring exons; and when introduced into a primate cell (e.g., a hair cell or a supporting cell of the inner ear) the at least two different vectors undergo concatamerization or homologous recombination with each other, thereby forming a recombined nucleic acid that encodes a full-length hair cell differentiation protein that is expressed in the primate cell.

Also provided herein are compositions including two different nucleic acid vectors, where: a first nucleic acid vector of the two different nucleic acid vectors includes a promoter, a first coding sequence that encodes an N-terminal portion of a hair cell differentiation protein positioned 3′ of the promoter, and a splicing donor signal sequence positioned at the 3′ end of the first coding sequence; and a second nucleic acid vector of the two different nucleic acid vectors includes a splicing acceptor signal sequence, a second coding sequence that encodes a C-terminal portion of a hair cell differentiation protein positioned at the 3′ end of the splicing acceptor signal sequence, and a polyadenylation sequence at the 3′ end of the second coding sequence; where each of the encoded portions is at least 30 amino acid residues in length, where the amino acid sequences of the encoded portions do not overlap, where no single vector of the two different vectors encodes the full-length hair cell differentiation protein, and, when the coding sequences are transcribed in a primate cell (e.g., a hair cell or a supporting cell of the inner ear), to produce RNA transcripts, splicing occurs between the splicing donor signal sequence on one transcript and the splicing acceptor signal sequence on the other transcript, thereby forming a recombined RNA molecule that encodes a full-length hair cell differentiation protein.

Also provided herein are compositions including: a first nucleic acid vector including a promoter, a first coding sequence that encodes an N-terminal portion of a hair cell differentiation protein positioned 3′ of the promoter, a splicing donor signal sequence positioned at the 3′ end of the first coding sequence, and a first detectable marker gene positioned 3′ of the splicing donor signal sequence; and a second nucleic acid vector, different from the first nucleic acid vector, including a second detectable marker gene, a splicing acceptor signal sequence positioned 3′ of the second detectable marker gene, a second coding sequence that encodes a C-terminal portion of a hair cell differentiation protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell differentiation protein, and, when the coding sequences are transcribed in a primate cell (e.g., a hair cell or a supporting cell of the inner ear) to produce RNA transcripts, splicing occurs between the splicing donor signal on one transcript and the splicing acceptor signal on the other transcript, thereby forming a recombined RNA molecule that encodes a full-length hair cell differentiation protein.

Also provided herein are compositions including: a first nucleic acid vector including a promoter, a first coding sequence that encodes an N-terminal portion of a hair cell differentiation protein positioned 3′ to the promoter, a splicing donor signal sequence positioned at the 3′ end of the first coding sequence, and a F1 phage recombinogenic region positioned 3′ to the splicing donor signal sequence; and a second nucleic acid vector, different from the first nucleic acid vector, including a second F1 phage recombinogenic region, a splicing acceptor signal sequence positioned 3′ of the second F1 phage recombinogenic region, a second coding sequence that encodes a C-terminal portion of a hair cell differentiation protein positioned at the 3′ end of the splicing acceptor signal sequence, and a polyadenylation sequence positioned at the 3′ end of the second coding sequence; where each of the encoded portions is at least 30 amino acid residues in length, where the respective amino acid sequences of the encoded portions do not overlap with each other, where no single vector of the two different vectors encodes the full-length hair cell differentiation protein, and, when the coding sequences are transcribed in a primate cell (e.g., a hair cell or a supporting cell of the inner ear) to produce RNA transcripts, splicing occurs between the splicing donor signal one transcript and the splicing acceptor signal on the other transcript, thereby forming a recombined RNA molecule that encodes a full-length hair cell differentiation protein.

Also provided herein are compositions including a single adeno-associated virus (AAV) vector, where the single AAV vector comprises a nucleic acid sequence that encodes a hair cell differentiation protein; and when introduced into a primate cell (e.g., a hair cell or a supporting cell of the inner ear), a nucleic acid encoding the hair cell differentiation protein is generated at the locus of the hair cell differentiation gene and the primate cell expresses the hair cell differentiation protein. Also provided herein are compositions including a single adeno-associated virus (AAV) vector that encodes an inhibitory nucleic acid that decreases the expression of a hair cell differentiation-suppressing protein in a primate cell (e.g., a hair cell or a supporting cell of the inner ear).

Also provided herein are methods of promoting differentiation of a supporting cell of an inner ear of a primate into a hair cell that include: administering to the inner ear of the primate a therapeutically effective amount of any of the compositions described herein, where the administering promotes differentiation of the supporting cell of the inner ear of the primate into a hair cell. Also provided herein are methods of increasing the expression level of a hair cell differentiation protein in a supporting cell of an inner ear of a primate that include: administering to the inner ear of the primate a therapeutically effective amount of any of the compositions described herein, where the administering results in an increase in the expression level of the hair cell differentiation protein in the supporting cell of the inner ear of the primate.

Also provided herein are methods of decreasing the expression level of a hair cell differentiation-suppressing protein in a supporting cell or a hair cell of an inner ear of a primate that include: administering to the inner ear of the primate a therapeutically effective amount of any of the compositions described herein, where the administering results in a decrease in the expression level of the hair cell differentiation-suppressing protein in the supporting cell or the hair cell of the inner ear of the primate.

Also provided herein are methods of increasing the number of functional hair cells in a primate in need thereof that include: administering to the inner ear of the primate a therapeutically effective amount of any of the compositions described herein. Also provided herein are methods of improving hearing in a primate in need thereof, the method comprising administering to the inner ear of the primate a therapeutically effective amount of any of the compositions described herein.

Also provided herein are methods of repairing a hair cell toxicity-inducing mutation in an endogenous hair cell differentiation gene locus in a supporting cell or a hair cell of an inner ear of a primate that include: administering to the inner ear of the primate a therapeutically effective amount of any of the compositions described herein, where the administering results in repair of the hair cell toxicity-inducing mutation in the endogenous hair cell differentiation gene locus in the supporting cell or the hair cell of the inner ear of the primate.

Also provided herein are methods of decreasing the risk of hearing loss due to hair cell loss or dysfunction in a primate in need thereof that include: administering to the inner ear of the primate a therapeutically effective amount of any of the compositions described herein. Also provided herein are methods that include introducing into a cochlea of a mammal a therapeutically effective amount of any of the compositions described herein.

Also provided are kits that include any of the compositions described herein.

Additional non-limiting aspects of the compositions, kits, and methods are described herein and can be used in any combination without limitation.

### Hair Cell Differentiation Genes

The term “hair cell differentiation gene” refers to a gene encoding a protein (e.g., a transcription factor) that positively contributes, either directly or indirectly, to hair cell differentiation and viability in a primate (e.g., a human). Non-limiting examples of hair cell differentiation genes include: ATOH1, POU4F3, CTNNB1, NOG, GFI-1, NTF3, and BDNF.

The term “mutation in a hair cell differentiation gene” refers to a modification in a wildtype hair cell differentiation gene that results in the production of a hair cell differentiation protein having one or more of: a deletion in one or more amino acids, one or more amino acid substitutions, and one or more amino acid insertions as compared to the wildtype hair cell differentiation protein, and/or results in a decrease in the expressed level of the encoded hair cell differentiation protein in a primate cell as compared to the expressed level of the encoded hair cell differentiation protein in a primate cell not having a mutation. In some embodiments, a mutation can result in the production of a hair cell differentiation protein having a deletion in one or more amino acids (e.g., 2, 3, 4, 5, 6, 7, 8, 9, 10, 11, 12, 13, 14, 15 16, 17, 18, 19, or 20 amino acids). In some embodiments, the mutation can result in a frameshift in the hair cell differentiation gene. The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., results in a premature stop codon in an exon of the gene). A nonsense mutation can result in the production of a truncated protein (as compared to a corresponding wildtype protein) that may or may not be functional. In some embodiments, the mutation can result in the loss (or a decrease in the level) of expression of hair cell differentiation mRNA or hair cell differentiation protein, or both the mRNA and protein. In some embodiments, the mutation can result in the production of an altered hair cell differentiation protein having a loss or decrease in one or more biological activities (functions) as compared to a wildtype hair cell differentiation protein.

In some embodiments, the mutation is an insertion of one or more nucleotides into a hair cell differentiation gene. In some embodiments, the mutation is in a regulatory sequence of the hair cell differentiation gene, i.e., a portion of the gene that is not coding sequence. In some embodiments, a mutation in a regulatory sequence may be in a promoter or enhancer region and prevent or reduce the proper transcription of the hair cell differentiation gene.

For example, an active hair cell differentiation protein can include a sequence of a wildtype, full-length hair cell differentiation protein (e.g., a wildtype, human, full-length hair cell differentiation protein) including 1 amino acid substitution to about 160 amino acid substitutions, 1 amino acid substitution to about 155 amino acid substitutions, 1 amino acid substitution to about 150 amino acid substitutions, 1 amino acid substitution to about 145 amino acid substitutions, 1 amino acid substitution to about 140 amino acid substitutions, 1 amino acid substitution to about 135 amino acid substitutions, 1 amino acid substitution to about 130 amino acid substitutions, 1 amino acid substitution to about 125 amino acid substitutions, 1 amino acid substitution to about 120 amino acid substitutions, 1 amino acid substitution to about 115 amino acid substitutions, 1 amino acid substitution to about 110 amino acid substitutions, 1 amino acid substitution to about 105 amino acid substitutions, 1 amino acid substitution to about 100 amino acid substitutions, 1 amino acid substitution to about 95 amino acid substitutions, 1 amino acid substitution to about 90 amino acid substitutions, 1 amino acid substitution to about 85 amino acid substitutions, 1 amino acid substitution to about 80 amino acid substitutions, 1 amino acid substitution to about 75 amino acid substitutions, 1 amino acid substitution to about 70 amino acid substitutions, 1 amino acid substitution to about 65 amino acid substitutions, 1 amino acid substitution to about 60 amino acid substitutions, 1 amino acid substitution to about 55 amino acid substitutions, 1 amino acid substitution to about 50 amino acid substitutions, 1 amino acid substitution to about 45 amino acid substitutions, 1 amino acid substitution to about 40 amino acid substitutions, 1 amino acid substitution to about 35 amino acid substitutions, 1 amino acid substitution to about 30 amino acid substitutions, 1 amino acid substitution to about 25 amino acid substitutions, 1 amino acid substitution to about 20 amino acid substitutions, 1 amino acid substitution to about 15 amino acid substitutions, 1 amino acid substitution to about 10 amino acid substitutions, 1 amino acid substitution to about 9 amino acid substitutions, 1 amino acid substitution to about 8 amino acid substitutions, 1 amino acid substitution to about 7 amino acid substitutions, 1 amino acid substitution to about 6 amino acid substitutions, 1 amino acid substitution to about 5 amino acid substitutions, 1 amino acid substitution to about 4 amino acid substitutions, 1 amino acid substitution to about 3 amino acid substitutions, between about 2 amino acid substitutions to about 160 amino acid substitutions, about 2 amino acid substitutions to about 155 amino acid substitutions, about 2 amino acid substitutions to about 150 amino acid substitutions, about 2 amino acid substitutions to about 145 amino acid substitutions, about 2 amino acid substitutions to about 140 amino acid substitutions, about 2 amino acid substitutions to about 135 amino acid substitutions, about 2 amino acid substitutions to about 130 amino acid substitutions, about 2 amino acid substitutions to about 125 amino acid substitutions, about 2 amino acid substitutions to about 120 amino acid substitutions, about 2 amino acid substitutions to about 115 amino acid substitutions, about 2 amino acid substitutions to about 110 amino acid substitutions, about 2 amino acid substitutions to about 105 amino acid substitutions, about 2 amino acid substitutions to about 100 amino acid substitutions, about 2 amino acid substitutions to about 95 amino acid substitutions, about 2 amino acid substitutions to about 90 amino acid substitutions, about 2 amino acid substitutions to about 85 amino acid substitutions, about 2 amino acid substitutions to about 80 amino acid substitutions, about 2 amino acid substitutions to about 75 amino acid substitutions, about 2 amino acid substitutions to about 70 amino acid substitutions, about 2 amino acid substitutions to about 65 amino acid substitutions, about 2 amino acid substitutions to about 60 amino acid substitutions, about 2 amino acid substitutions to about 55 amino acid substitutions, about 2 amino acid substitutions to about 50 amino acid substitutions, about 2 amino acid substitutions to about 45 amino acid substitutions, about 2 amino acid substitutions to about 40 amino acid substitutions, about 2 amino acid substitutions to about 35 amino acid substitutions, about 2 amino acid substitutions to about 30 amino acid substitutions, about 2 amino acid substitutions to about 25 amino acid substitutions, about 2 amino acid substitutions to about 20 amino acid substitutions, about 2 amino acid substitutions to about 15 amino acid substitutions, about 2 amino acid substitutions to about 10 amino acid substitutions, about 2 amino acid substitutions to about 9 amino acid substitutions, about 2 amino acid substitutions to about 8 amino acid substitutions, about 2 amino acid substitutions to about 7 amino acid substitutions, about 2 amino acid substitutions to about 6 amino acid substitutions, about 2 amino acid substitutions to about 5 amino acid substitutions, about 2 amino acid substitutions to about 4 amino acid substitutions, between about 3 amino acid substitutions to about 160 amino acid substitutions, about 3 amino acid substitutions to about 155 amino acid substitutions, about 3 amino acid substitutions to about 150 amino acid substitutions, about 3 amino acid substitutions to about 145 amino acid substitutions, about 3 amino acid substitutions to about 140 amino acid substitutions, about 3 amino acid substitutions to about 135 amino acid substitutions, about 3 amino acid substitutions to about 130 amino acid substitutions, about 3 amino acid substitutions to about 125 amino acid substitutions, about 3 amino acid substitutions to about 120 amino acid substitutions, about 3 amino acid substitutions to about 115 amino acid substitutions, about 3 amino acid substitutions to about 110 amino acid substitutions, about 3 amino acid substitutions to about 105 amino acid substitutions, about 3 amino acid substitutions to about 100 amino acid substitutions, about 3 amino acid substitutions to about 95 amino acid substitutions, about 3 amino acid substitutions to about 90 amino acid substitutions, about 3 amino acid substitutions to about 85 amino acid substitutions, about 3 amino acid substitutions to about 80 amino acid substitutions, about 3 amino acid substitutions to about 75 amino acid substitutions, about 3 amino acid substitutions to about 70 amino acid substitutions, about 3 amino acid substitutions to about 65 amino acid substitutions, about 3 amino acid substitutions to about 60 amino acid substitutions, about 3 amino acid substitutions to about 55 amino acid substitutions, about 3 amino acid substitutions to about 50 amino acid substitutions, about 3 amino acid substitutions to about 45 amino acid substitutions, about 3 amino acid substitutions to about 40 amino acid substitutions, about 3 amino acid substitutions to about 35 amino acid substitutions, about 3 amino acid substitutions to about 30 amino acid substitutions, about 3 amino acid substitutions to about 25 amino acid substitutions, about 3 amino acid substitutions to about 20 amino acid substitutions, about 3 amino acid substitutions to about 15 amino acid substitutions, about 3 amino acid substitutions to about 10 amino acid substitutions, about 3 amino acid substitutions to about 9 amino acid substitutions, about 3 amino acid substitutions to about 8 amino acid substitutions, about 3 amino acid substitutions to about 7 amino acid substitutions, about 3 amino acid substitutions to about 6 amino acid substitutions, about 3 amino acid substitutions to about 5 amino acid substitutions, between about 4 amino acid substitutions to about 160 amino acid substitutions, about 4 amino acid substitutions to about 155 amino acid substitutions, about 4 amino acid substitutions to about 150 amino acid substitutions, about 4 amino acid substitutions to about 145 amino acid substitutions, about 4 amino acid substitutions to about 140 amino acid substitutions, about 4 amino acid substitutions to about 135 amino acid substitutions, about 4 amino acid substitutions to about 130 amino acid substitutions, about 4 amino acid substitutions to about 125 amino acid substitutions, about 4 amino acid substitutions to about 120 amino acid substitutions, about 4 amino acid substitutions to about 115 amino acid substitutions, about 4 amino acid substitutions to about 110 amino acid substitutions, about 4 amino acid substitutions to about 105 amino acid substitutions, about 4 amino acid substitutions to about 100 amino acid substitutions, about 4 amino acid substitutions to about 95 amino acid substitutions, about 4 amino acid substitutions to about 90 amino acid substitutions, about 4 amino acid substitutions to about 85 amino acid substitutions, about 4 amino acid substitutions to about 80 amino acid substitutions, about 4 amino acid substitutions to about 75 amino acid substitutions, about 4 amino acid substitutions to about 70 amino acid substitutions, about 4 amino acid substitutions to about 65 amino acid substitutions, about 4 amino acid substitutions to about 60 amino acid substitutions, about 4 amino acid substitutions to about 55 amino acid substitutions, about 4 amino acid substitutions to about 50 amino acid substitutions, about 4 amino acid substitutions to about 45 amino acid substitutions, about 4 amino acid substitutions to about 40 amino acid substitutions, about 4 amino acid substitutions to about 35 amino acid substitutions, about 4 amino acid substitutions to about 30 amino acid substitutions, about 4 amino acid substitutions to about 25 amino acid substitutions, about 4 amino acid substitutions to about 20 amino acid substitutions, about 4 amino acid substitutions to about 15 amino acid substitutions, about 4 amino acid substitutions to about 10 amino acid substitutions, about 4 amino acid substitutions to about 9 amino acid substitutions, about 4 amino acid substitutions to about 8 amino acid substitutions, about 4 amino acid substitutions to about 7 amino acid substitutions, about 4 amino acid substitutions to about 6 amino acid substitutions, between about 5 amino acid substitutions to about 160 amino acid substitutions, about 5 amino acid substitutions to about 155 amino acid substitutions, about 5 amino acid substitutions to about 150 amino acid substitutions, about 5 amino acid substitutions to about 145 amino acid substitutions, about 5 amino acid substitutions to about 140 amino acid substitutions, about 5 amino acid substitutions to about 135 amino acid substitutions, about 5 amino acid substitutions to about 130 amino acid substitutions, about 5 amino acid substitutions to about 125 amino acid substitutions, about 5 amino acid substitutions to about 120 amino acid substitutions, about 5 amino acid substitutions to about 115 amino acid substitutions, about 5 amino acid substitutions to about 110 amino acid substitutions, about 5 amino acid substitutions to about 105 amino acid substitutions, about 5 amino acid substitutions to about 100 amino acid substitutions, about 5 amino acid substitutions to about 95 amino acid substitutions, about 5 amino acid substitutions to about 90 amino acid substitutions, about 5 amino acid substitutions to about 85 amino acid substitutions, about 5 amino acid substitutions to about 80 amino acid substitutions, about 5 amino acid substitutions to about 75 amino acid substitutions, about 5 amino acid substitutions to about 70 amino acid substitutions, about 5 amino acid substitutions to about 65 amino acid substitutions, about 5 amino acid substitutions to about 60 amino acid substitutions, about 5 amino acid substitutions to about 55 amino acid substitutions, about 5 amino acid substitutions to about 50 amino acid substitutions, about 5 amino acid substitutions to about 45 amino acid substitutions, about 5 amino acid substitutions to about 40 amino acid substitutions, about 5 amino acid substitutions to about 35 amino acid substitutions, about 5 amino acid substitutions to about 30 amino acid substitutions, about 5 amino acid substitutions to about 25 amino acid substitutions, about 5 amino acid substitutions to about 20 amino acid substitutions, about 5 amino acid substitutions to about 15 amino acid substitutions, about 5 amino acid substitutions to about 10 amino acid substitutions, about 5 amino acid substitutions to about 9 amino acid substitutions, about 5 amino acid substitutions to about 8 amino acid substitutions, about 5 amino acid substitutions to about 7 amino acid substitutions, between about 6 amino acid substitutions to about 160 amino acid substitutions, about 6 amino acid substitutions to about 155 amino acid substitutions, about 6 amino acid substitutions to about 150 amino acid substitutions, about 6 amino acid substitutions to about 145 amino acid substitutions, about 6 amino acid substitutions to about 140 amino acid substitutions, about 6 amino acid substitutions to about 135 amino acid substitutions, about 6 amino acid substitutions to about 130 amino acid substitutions, about 6 amino acid substitutions to about 125 amino acid substitutions, about 6 amino acid substitutions to about 120 amino acid substitutions, about 6 amino acid substitutions to about 115 amino acid substitutions, about 6 amino acid substitutions to about 110 amino acid substitutions, about 6 amino acid substitutions to about 105 amino acid substitutions, about 6 amino acid substitutions to about 100 amino acid substitutions, about 6 amino acid substitutions to about 95 amino acid substitutions, about 6 amino acid substitutions to about 90 amino acid substitutions, about 6 amino acid substitutions to about 85 amino acid substitutions, about 6 amino acid substitutions to about 80 amino acid substitutions, about 6 amino acid substitutions to about 75 amino acid substitutions, about 6 amino acid substitutions to about 70 amino acid substitutions, about 6 amino acid substitutions to about 65 amino acid substitutions, about 6 amino acid substitutions to about 60 amino acid substitutions, about 6 amino acid substitutions to about 55 amino acid substitutions, about 6 amino acid substitutions to about 50 amino acid substitutions, about 6 amino acid substitutions to about 45 amino acid substitutions, about 6 amino acid substitutions to about 40 amino acid substitutions, about 6 amino acid substitutions to about 35 amino acid substitutions, about 6 amino acid substitutions to about 30 amino acid substitutions, about 6 amino acid substitutions to about 25 amino acid substitutions, about 6 amino acid substitutions to about 20 amino acid substitutions, about 6 amino acid substitutions to about 15 amino acid substitutions, about 6 amino acid substitutions to about 10 amino acid substitutions, about 6 amino acid substitutions to about 9 amino acid substitutions, about 6 amino acid substitutions to about 8 amino acid substitutions, between about 7 amino acid substitutions to about 160 amino acid substitutions, about 7 amino acid substitutions to about 155 amino acid substitutions, about 7 amino acid substitutions to about 150 amino acid substitutions, about 7 amino acid substitutions to about 145 amino acid substitutions, about 7 amino acid substitutions to about 140 amino acid substitutions, about 7 amino acid substitutions to about 135 amino acid substitutions, about 7 amino acid substitutions to about 130 amino acid substitutions, about 7 amino acid substitutions to about 125 amino acid substitutions, about 7 amino acid substitutions to about 120 amino acid substitutions, about 7 amino acid substitutions to about 115 amino acid substitutions, about 7 amino acid substitutions to about 110 amino acid substitutions, about 7 amino acid substitutions to about 105 amino acid substitutions, about 7 amino acid substitutions to about 100 amino acid substitutions, about 7 amino acid substitutions to about 95 amino acid substitutions, about 7 amino acid substitutions to about 90 amino acid substitutions, about 7 amino acid substitutions to about 85 amino acid substitutions, about 7 amino acid substitutions to about 80 amino acid substitutions, about 7 amino acid substitutions to about 75 amino acid substitutions, about 7 amino acid substitutions to about 70 amino acid substitutions, about 7 amino acid substitutions to about 65 amino acid substitutions, about 7 amino acid substitutions to about 60 amino acid substitutions, about 7 amino acid substitutions to about 55 amino acid substitutions, about 7 amino acid substitutions to about 50 amino acid substitutions, about 7 amino acid substitutions to about 45 amino acid substitutions, about 7 amino acid substitutions to about 40 amino acid substitutions, about 7 amino acid substitutions to about 35 amino acid substitutions, about 7 amino acid substitutions to about 30 amino acid substitutions, about 7 amino acid substitutions to about 25 amino acid substitutions, about 7 amino acid substitutions to about 20 amino acid substitutions, about 7 amino acid substitutions to about 15 amino acid substitutions, about 7 amino acid substitutions to about 10 amino acid substitutions, about 7 amino acid substitutions to about 9 amino acid substitutions, between about 8 amino acid substitutions to about 160 amino acid substitutions, about 8 amino acid substitutions to about 155 amino acid substitutions, about 8 amino acid substitutions to about 150 amino acid substitutions, about 8 amino acid substitutions to about 145 amino acid substitutions, about 8 amino acid substitutions to about 140 amino acid substitutions, about 8 amino acid substitutions to about 135 amino acid substitutions, about 8 amino acid substitutions to about 130 amino acid substitutions, about 8 amino acid substitutions to about 125 amino acid substitutions, about 8 amino acid substitutions to about 120 amino acid substitutions, about 8 amino acid substitutions to about 115 amino acid substitutions, about 8 amino acid substitutions to about 110 amino acid substitutions, about 8 amino acid substitutions to about 105 amino acid substitutions, about 8 amino acid substitutions to about 100 amino acid substitutions, about 8 amino acid substitutions to about 95 amino acid substitutions, about 8 amino acid substitutions to about 90 amino acid substitutions, about 8 amino acid substitutions to about 85 amino acid substitutions, about 8 amino acid substitutions to about 80 amino acid substitutions, about 8 amino acid substitutions to about 75 amino acid substitutions, about 8 amino acid substitutions to about 70 amino acid substitutions, about 8 amino acid substitutions to about 65 amino acid substitutions, about 8 amino acid substitutions to about 60 amino acid substitutions, about 8 amino acid substitutions to about 55 amino acid substitutions, about 8 amino acid substitutions to about 50 amino acid substitutions, about 8 amino acid substitutions to about 45 amino acid substitutions, about 8 amino acid substitutions to about 40 amino acid substitutions, about 8 amino acid substitutions to about 35 amino acid substitutions, about 8 amino acid substitutions to about 30 amino acid substitutions, about 8 amino acid substitutions to about 25 amino acid substitutions, about 8 amino acid substitutions to about 20 amino acid substitutions, about 8 amino acid substitutions to about 15 amino acid substitutions, about 8 amino acid substitutions to about 10 amino acid substitutions, between about 10 amino acid substitutions to about 160 amino acid substitutions, about 10 amino acid substitutions to about 155 amino acid substitutions, about 10 amino acid substitutions to about 150 amino acid substitutions, about 10 amino acid substitutions to about 145 amino acid substitutions, about 10 amino acid substitutions to about 140 amino acid substitutions, about 10 amino acid substitutions to about 135 amino acid substitutions, about 10 amino acid substitutions to about 130 amino acid substitutions, about 10 amino acid substitutions to about 125 amino acid substitutions, about 10 amino acid substitutions to about 120 amino acid substitutions, about 10 amino acid substitutions to about 115 amino acid substitutions, about 10 amino acid substitutions to about 110 amino acid substitutions, about 10 amino acid substitutions to about 105 amino acid substitutions, about 10 amino acid substitutions to about 100 amino acid substitutions, about 10 amino acid substitutions to about 95 amino acid substitutions, about 10 amino acid substitutions to about 90 amino acid substitutions, about 10 amino acid substitutions to about 85 amino acid substitutions, about 10 amino acid substitutions to about 80 amino acid substitutions, about 10 amino acid substitutions to about 75 amino acid substitutions, about 10 amino acid substitutions to about 70 amino acid substitutions, about 10 amino acid substitutions to about 65 amino acid substitutions, about 10 amino acid substitutions to about 60 amino acid substitutions, about 10 amino acid substitutions to about 55 amino acid substitutions, about 10 amino acid substitutions to about 50 amino acid substitutions, about 10 amino acid substitutions to about 45 amino acid substitutions, about 10 amino acid substitutions to about 40 amino acid substitutions, about 10 amino acid substitutions to about 35 amino acid substitutions, about 10 amino acid substitutions to about 30 amino acid substitutions, about 10 amino acid substitutions to about 25 amino acid substitutions, about 10 amino acid substitutions to about 20 amino acid substitutions, about 10 amino acid substitutions to about 15 amino acid substitutions, between about 15 amino acid substitutions to about 160 amino acid substitutions, about 15 amino acid substitutions to about 155 amino acid substitutions, about 15 amino acid substitutions to about 150 amino acid substitutions, about 15 amino acid substitutions to about 145 amino acid substitutions, about 15 amino acid substitutions to about 140 amino acid substitutions, about 15 amino acid substitutions to about 135 amino acid substitutions, about 15 amino acid substitutions to about 130 amino acid substitutions, about 15 amino acid substitutions to about 125 amino acid substitutions, about 15 amino acid substitutions to about 120 amino acid substitutions, about 15 amino acid substitutions to about 115 amino acid substitutions, about 15 amino acid substitutions to about 110 amino acid substitutions, about 15 amino acid substitutions to about 105 amino acid substitutions, about 15 amino acid substitutions to about 100 amino acid substitutions, about 15 amino acid substitutions to about 95 amino acid substitutions, about 15 amino acid substitutions to about 90 amino acid substitutions, about 15 amino acid substitutions to about 85 amino acid substitutions, about 15 amino acid substitutions to about 80 amino acid substitutions, about 15 amino acid substitutions to about 75 amino acid substitutions, about 15 amino acid substitutions to about 70 amino acid substitutions, about 15 amino acid substitutions to about 65 amino acid substitutions, about 15 amino acid substitutions to about 60 amino acid substitutions, about 15 amino acid substitutions to about 55 amino acid substitutions, about 15 amino acid substitutions to about 50 amino acid substitutions, about 15 amino acid substitutions to about 45 amino acid substitutions, about 15 amino acid substitutions to about 40 amino acid substitutions, about 15 amino acid substitutions to about 35 amino acid substitutions, about 15 amino acid substitutions to about 30 amino acid substitutions, about 15 amino acid substitutions to about 25 amino acid substitutions, about 15 amino acid substitutions to about 20 amino acid substitutions, between about 20 amino acid substitutions to about 160 amino acid substitutions, about 20 amino acid substitutions to about 155 amino acid substitutions, about 20 amino acid substitutions to about 150 amino acid substitutions, about 20 amino acid substitutions to about 145 amino acid substitutions, about 20 amino acid substitutions to about 140 amino acid substitutions, about 20 amino acid substitutions to about 135 amino acid substitutions, about 20 amino acid substitutions to about 130 amino acid substitutions, about 20 amino acid substitutions to about 125 amino acid substitutions, about 20 amino acid substitutions to about 120 amino acid substitutions, about 20 amino acid substitutions to about 115 amino acid substitutions, about 20 amino acid substitutions to about 110 amino acid substitutions, about 20 amino acid substitutions to about 105 amino acid substitutions, about 20 amino acid substitutions to about 100 amino acid substitutions, about 20 amino acid substitutions to about 95 amino acid substitutions, about 20 amino acid substitutions to about 90 amino acid substitutions, about 20 amino acid substitutions to about 85 amino acid substitutions, about 20 amino acid substitutions to about 80 amino acid substitutions, about 20 amino acid substitutions to about 75 amino acid substitutions, about 20 amino acid substitutions to about 70 amino acid substitutions, about 20 amino acid substitutions to about 65 amino acid substitutions, about 20 amino acid substitutions to about 60 amino acid substitutions, about 20 amino acid substitutions to about 55 amino acid substitutions, about 20 amino acid substitutions to about 50 amino acid substitutions, about 20 amino acid substitutions to about 45 amino acid substitutions, about 20 amino acid substitutions to about 40 amino acid substitutions, about 20 amino acid substitutions to about 35 amino acid substitutions, about 20 amino acid substitutions to about 30 amino acid substitutions, about 20 amino acid substitutions to about 25 amino acid substitutions, between about 25 amino acid substitutions to about 160 amino acid substitutions, about 25 amino acid substitutions to about 155 amino acid substitutions, about 25 amino acid substitutions to about 150 amino acid substitutions, about 25 amino acid substitutions to about 145 amino acid substitutions, about 25 amino acid substitutions to about 140 amino acid substitutions, about 25 amino acid substitutions to about 135 amino acid substitutions, about 25 amino acid substitutions to about 130 amino acid substitutions, about 25 amino acid substitutions to about 125 amino acid substitutions, about 25 amino acid substitutions to about 120 amino acid substitutions, about 25 amino acid substitutions to about 115 amino acid substitutions, about 25 amino acid substitutions to about 110 amino acid substitutions, about 25 amino acid substitutions to about 105 amino acid substitutions, about 25 amino acid substitutions to about 100 amino acid substitutions, about 25 amino acid substitutions to about 95 amino acid substitutions, about 25 amino acid substitutions to about 90 amino acid substitutions, about 25 amino acid substitutions to about 85 amino acid substitutions, about 25 amino acid substitutions to about 80 amino acid substitutions, about 25 amino acid substitutions to about 75 amino acid substitutions, about 25 amino acid substitutions to about 70 amino acid substitutions, about 25 amino acid substitutions to about 65 amino acid substitutions, about 25 amino acid substitutions to about 60 amino acid substitutions, about 25 amino acid substitutions to about 55 amino acid substitutions, about 25 amino acid substitutions to about 50 amino acid substitutions, about 25 amino acid substitutions to about 45 amino acid substitutions, about 25 amino acid substitutions to about 40 amino acid substitutions, about 25 amino acid substitutions to about 35 amino acid substitutions, about 25 amino acid substitutions to about 30 amino acid substitutions, between about 30 amino acid substitutions to about 160 amino acid substitutions, about 30 amino acid substitutions to about 155 amino acid substitutions, about 30 amino acid substitutions to about 150 amino acid substitutions, about 30 amino acid substitutions to about 145 amino acid substitutions, about 30 amino acid substitutions to about 140 amino acid substitutions, about 30 amino acid substitutions to about 135 amino acid substitutions, about 30 amino acid substitutions to about 130 amino acid substitutions, about 30 amino acid substitutions to about 125 amino acid substitutions, about 30 amino acid substitutions to about 120 amino acid substitutions, about 30 amino acid substitutions to about 115 amino acid substitutions, about 30 amino acid substitutions to about 110 amino acid substitutions, about 30 amino acid substitutions to about 105 amino acid substitutions, about 30 amino acid substitutions to about 100 amino acid substitutions, about 30 amino acid substitutions to about 95 amino acid substitutions, about 30 amino acid substitutions to about 90 amino acid substitutions, about 30 amino acid substitutions to about 85 amino acid substitutions, about 30 amino acid substitutions to about 80 amino acid substitutions, about 30 amino acid substitutions to about 75 amino acid substitutions, about 30 amino acid substitutions to about 70 amino acid substitutions, about 30 amino acid substitutions to about 65 amino acid substitutions, about 30 amino acid substitutions to about 60 amino acid substitutions, about 30 amino acid substitutions to about 55 amino acid substitutions, about 30 amino acid substitutions to about 50 amino acid substitutions, about 30 amino acid substitutions to about 45 amino acid substitutions, about 30 amino acid substitutions to about 40 amino acid substitutions, about 30 amino acid substitutions to about 35 amino acid substitutions, between about 35 amino acid substitutions to about 160 amino acid substitutions, about 35 amino acid substitutions to about 155 amino acid substitutions, about 35 amino acid substitutions to about 150 amino acid substitutions, about 35 amino acid substitutions to about 145 amino acid substitutions, about 35 amino acid substitutions to about 140 amino acid substitutions, about 35 amino acid substitutions to about 135 amino acid substitutions, about 35 amino acid substitutions to about 130 amino acid substitutions, about 35 amino acid substitutions to about 125 amino acid substitutions, about 35 amino acid substitutions to about 120 amino acid substitutions, about 35 amino acid substitutions to about 115 amino acid substitutions, about 35 amino acid substitutions to about 110 amino acid substitutions, about 35 amino acid substitutions to about 105 amino acid substitutions, about 35 amino acid substitutions to about 100 amino acid substitutions, about 35 amino acid substitutions to about 95 amino acid substitutions, about 35 amino acid substitutions to about 90 amino acid substitutions, about 35 amino acid substitutions to about 85 amino acid substitutions, about 35 amino acid substitutions to about 80 amino acid substitutions, about 35 amino acid substitutions to about 75 amino acid substitutions, about 35 amino acid substitutions to about 70 amino acid substitutions, about 35 amino acid substitutions to about 65 amino acid substitutions, about 35 amino acid substitutions to about 60 amino acid substitutions, about 35 amino acid substitutions to about 55 amino acid substitutions, about 35 amino acid substitutions to about 50 amino acid substitutions, about 35 amino acid substitutions to about 45 amino acid substitutions, about 35 amino acid substitutions to about 40 amino acid substitutions, between about 40 amino acid substitutions to about 160 amino acid substitutions, about 40 amino acid substitutions to about 155 amino acid substitutions, about 40 amino acid substitutions to about 150 amino acid substitutions, about 40 amino acid substitutions to about 145 amino acid substitutions, about 40 amino acid substitutions to about 140 amino acid substitutions, about 40 amino acid substitutions to about 135 amino acid substitutions, about 40 amino acid substitutions to about 130 amino acid substitutions, about 40 amino acid substitutions to about 125 amino acid substitutions, about 40 amino acid substitutions to about 120 amino acid substitutions, about 40 amino acid substitutions to about 115 amino acid substitutions, about 40 amino acid substitutions to about 110 amino acid substitutions, about 40 amino acid substitutions to about 105 amino acid substitutions, about 40 amino acid substitutions to about 100 amino acid substitutions, about 40 amino acid substitutions to about 95 amino acid substitutions, about 40 amino acid substitutions to about 90 amino acid substitutions, about 40 amino acid substitutions to about 85 amino acid substitutions, about 40 amino acid substitutions to about 80 amino acid substitutions, about 40 amino acid substitutions to about 75 amino acid substitutions, about 40 amino acid substitutions to about 70 amino acid substitutions, about 40 amino acid substitutions to about 65 amino acid substitutions, about 40 amino acid substitutions to about 60 amino acid substitutions, about 40 amino acid substitutions to about 55 amino acid substitutions, about 40 amino acid substitutions to about 50 amino acid substitutions, about 40 amino acid substitutions to about 45 amino acid substitutions, between about 45 amino acid substitutions to about 160 amino acid substitutions, about 45 amino acid substitutions to about 155 amino acid substitutions, about 45 amino acid substitutions to about 150 amino acid substitutions, about 45 amino acid substitutions to about 145 amino acid substitutions, about 45 amino acid substitutions to about 140 amino acid substitutions, about 45 amino acid substitutions to about 135 amino acid substitutions, about 45 amino acid substitutions to about 130 amino acid substitutions, about 45 amino acid substitutions to about 125 amino acid substitutions, about 45 amino acid substitutions to about 120 amino acid substitutions, about 45 amino acid substitutions to about 115 amino acid substitutions, about 45 amino acid substitutions to about 110 amino acid substitutions, about 45 amino acid substitutions to about 105 amino acid substitutions, about 45 amino acid substitutions to about 100 amino acid substitutions, about 45 amino acid substitutions to about 95 amino acid substitutions, about 45 amino acid substitutions to about 90 amino acid substitutions, about 45 amino acid substitutions to about 85 amino acid substitutions, about 45 amino acid substitutions to about 80 amino acid substitutions, about 45 amino acid substitutions to about 75 amino acid substitutions, about 45 amino acid substitutions to about 70 amino acid substitutions, about 45 amino acid substitutions to about 65 amino acid substitutions, about 45 amino acid substitutions to about 60 amino acid substitutions, about 45 amino acid substitutions to about 55 amino acid substitutions, about 45 amino acid substitutions to about 50 amino acid substitutions, between about 50 amino acid substitutions to about 160 amino acid substitutions, about 50 amino acid substitutions to about 155 amino acid substitutions, about 50 amino acid substitutions to about 150 amino acid substitutions, about 50 amino acid substitutions to about 145 amino acid substitutions, about 50 amino acid substitutions to about 140 amino acid substitutions, about 50 amino acid substitutions to about 135 amino acid substitutions, about 50 amino acid substitutions to about 130 amino acid substitutions, about 50 amino acid substitutions to about 125 amino acid substitutions, about 50 amino acid substitutions to about 120 amino acid substitutions, about 50 amino acid substitutions to about 115 amino acid substitutions, about 50 amino acid substitutions to about 110 amino acid substitutions, about 50 amino acid substitutions to about 105 amino acid substitutions, about 50 amino acid substitutions to about 100 amino acid substitutions, about 50 amino acid substitutions to about 95 amino acid substitutions, about 50 amino acid substitutions to about 90 amino acid substitutions, about 50 amino acid substitutions to about 85 amino acid substitutions, about 50 amino acid substitutions to about 80 amino acid substitutions, about 50 amino acid substitutions to about 75 amino acid substitutions, about 50 amino acid substitutions to about 70 amino acid substitutions, about 50 amino acid substitutions to about 65 amino acid substitutions, about 50 amino acid substitutions to about 60 amino acid substitutions, about 50 amino acid substitutions to about 55 amino acid substitutions, between about 60 amino acid substitutions to about 160 amino acid substitutions, about 60 amino acid substitutions to about 155 amino acid substitutions, about 60 amino acid substitutions to about 150 amino acid substitutions, about 60 amino acid substitutions to about 145 amino acid substitutions, about 60 amino acid substitutions to about 140 amino acid substitutions, about 60 amino acid substitutions to about 135 amino acid substitutions, about 60 amino acid substitutions to about 130 amino acid substitutions, about 60 amino acid substitutions to about 125 amino acid substitutions, about 60 amino acid substitutions to about 120 amino acid substitutions, about 60 amino acid substitutions to about 115 amino acid substitutions, about 60 amino acid substitutions to about 110 amino acid substitutions, about 60 amino acid substitutions to about 105 amino acid substitutions, about 60 amino acid substitutions to about 100 amino acid substitutions, about 60 amino acid substitutions to about 95 amino acid substitutions, about 60 amino acid substitutions to about 90 amino acid substitutions, about 60 amino acid substitutions to about 85 amino acid substitutions, about 60 amino acid substitutions to about 80 amino acid substitutions, about 60 amino acid substitutions to about 75 amino acid substitutions, about 60 amino acid substitutions to about 70 amino acid substitutions, about 60 amino acid substitutions to about 65 amino acid substitutions, between about 70 amino acid substitutions to about 160 amino acid substitutions, about 70 amino acid substitutions to about 155 amino acid substitutions, about 70 amino acid substitutions to about 150 amino acid substitutions, about 70 amino acid substitutions to about 145 amino acid substitutions, about 70 amino acid substitutions to about 140 amino acid substitutions, about 70 amino acid substitutions to about 135 amino acid substitutions, about 70 amino acid substitutions to about 130 amino acid substitutions, about 70 amino acid substitutions to about 125 amino acid substitutions, about 70 amino acid substitutions to about 120 amino acid substitutions, about 70 amino acid substitutions to about 115 amino acid substitutions, about 70 amino acid substitutions to about 110 amino acid substitutions, about 70 amino acid substitutions to about 105 amino acid substitutions, about 70 amino acid substitutions to about 100 amino acid substitutions, about 70 amino acid substitutions to about 95 amino acid substitutions, about 70 amino acid substitutions to about 90 amino acid substitutions, about 70 amino acid substitutions to about 85 amino acid substitutions, about 70 amino acid substitutions to about 80 amino acid substitutions, about 70 amino acid substitutions to about 75 amino acid substitutions, between about 80 amino acid substitutions to about 160 amino acid substitutions, about 80 amino acid substitutions to about 155 amino acid substitutions, about 80 amino acid substitutions to about 150 amino acid substitutions, about 80 amino acid substitutions to about 145 amino acid substitutions, about 80 amino acid substitutions to about 140 amino acid substitutions, about 80 amino acid substitutions to about 135 amino acid substitutions, about 80 amino acid substitutions to about 130 amino acid substitutions, about 80 amino acid substitutions to about 125 amino acid substitutions, about 80 amino acid substitutions to about 120 amino acid substitutions, about 80 amino acid substitutions to about 115 amino acid substitutions, about 80 amino acid substitutions to about 110 amino acid substitutions, about 80 amino acid substitutions to about 105 amino acid substitutions, about 80 amino acid substitutions to about 100 amino acid substitutions, about 80 amino acid substitutions to about 95 amino acid substitutions, about 80 amino acid substitutions to about 90 amino acid substitutions, about 80 amino acid substitutions to about 85 amino acid substitutions, between about 90 amino acid substitutions to about 160 amino acid substitutions, about 90 amino acid substitutions to about 155 amino acid substitutions, about 90 amino acid substitutions to about 150 amino acid substitutions, about 90 amino acid substitutions to about 145 amino acid substitutions, about 90 amino acid substitutions to about 140 amino acid substitutions, about 90 amino acid substitutions to about 135 amino acid substitutions, about 90 amino acid substitutions to about 130 amino acid substitutions, about 90 amino acid substitutions to about 125 amino acid substitutions, about 90 amino acid substitutions to about 120 amino acid substitutions, about 90 amino acid substitutions to about 115 amino acid substitutions, about 90 amino acid substitutions to about 110 amino acid substitutions, about 90 amino acid substitutions to about 105 amino acid substitutions, about 90 amino acid substitutions to about 100 amino acid substitutions, about 90 amino acid substitutions to about 95 amino acid substitutions, between about 100 amino acid substitutions to about 160 amino acid substitutions, about 100 amino acid substitutions to about 155 amino acid substitutions, about 100 amino acid substitutions to about 150 amino acid substitutions, about 100 amino acid substitutions to about 145 amino acid substitutions, about 100 amino acid substitutions to about 140 amino acid substitutions, about 100 amino acid substitutions to about 135 amino acid substitutions, about 100 amino acid substitutions to about 130 amino acid substitutions, about 100 amino acid substitutions to about 125 amino acid substitutions, about 100 amino acid substitutions to about 120 amino acid substitutions, about 100 amino acid substitutions to about 115 amino acid substitutions, about 100 amino acid substitutions to about 110 amino acid substitutions, about 100 amino acid substitutions to about 105 amino acid substitutions, between about 110 amino acid substitutions to about 160 amino acid substitutions, about 110 amino acid substitutions to about 155 amino acid substitutions, about 110 amino acid substitutions to about 150 amino acid substitutions, about 110 amino acid substitutions to about 145 amino acid substitutions, about 110 amino acid substitutions to about 140 amino acid substitutions, about 110 amino acid substitutions to about 135 amino acid substitutions, about 110 amino acid substitutions to about 130 amino acid substitutions, about 110 amino acid substitutions to about 125 amino acid substitutions, about 110 amino acid substitutions to about 120 amino acid substitutions, about 110 amino acid substitutions to about 115 amino acid substitutions, between about 120 amino acid substitutions to about 160 amino acid substitutions, about 120 amino acid substitutions to about 155 amino acid substitutions, about 120 amino acid substitutions to about 150 amino acid substitutions, about 120 amino acid substitutions to about 145 amino acid substitutions, about 120 amino acid substitutions to about 140 amino acid substitutions, about 120 amino acid substitutions to about 135 amino acid substitutions, about 120 amino acid substitutions to about 130 amino acid substitutions, about 120 amino acid substitutions to about 125 amino acid substitutions, between about 130 amino acid substitutions to about 160 amino acid substitutions, about 130 amino acid substitutions to about 155 amino acid substitutions, about 130 amino acid substitutions to about 150 amino acid substitutions, about 130 amino acid substitutions to about 145 amino acid substitutions, about 130 amino acid substitutions to about 140 amino acid substitutions, about 130 amino acid substitutions to about 135 amino acid substitutions, between about 140 amino acid substitutions to about 160 amino acid substitutions, about 140 amino acid substitutions to about 155 amino acid substitutions, about 140 amino acid substitutions to about 150 amino acid substitutions, about 140 amino acid substitutions to about 145 amino acid substitutions, between about 150 amino acid substitutions to about 160 amino acid substitutions, or about 150 amino acid substitutions to about 155 amino acid substitutions. One skilled in the art would appreciate that amino acids that are not conserved between wildtype hair cell differentiation proteins from different species can be mutated without losing activity, while those amino acids that are conserved between wildtype hair cell differentiation proteins from different species should not be mutated as they are more likely (than amino acids that are not conserved between different species) to be involved in activity.

An active hair cell differentiation protein can include, e.g., a sequence of a wildtype, full-length hair cell differentiation protein (e.g., a wildtype, human, full-length hair cell differentiation protein) that has 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 9 amino acids, 1 amino acid to about 8 amino acids, 1 amino acid to about 7 amino acids, 1 amino acid to about 6 amino acids, 1 amino acid to about 5 amino acids, 1 amino acid to about 4 amino acids, 1 amino acid to about 3 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 50 amino acids, about 8 amino acids to about 45 amino acids, about 8 amino acids to about 40 amino acids, about 8 amino acids to about 35 amino acids, about 8 amino acids to about amino acids to about 20 amino acids, about 8 amino acids to about 15 amino acids, about 8 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 35 amino acids to about 50 amino acids, about 35 amino acids to about 45 amino acids, about 35 amino acids to about 40 amino acids, about 40 amino acids to about 50 amino acids, about 40 amino acids to about 45 amino acids, about 45 amino acids to about 50 amino acids, deleted. In some embodiments where two or more amino acids are deleted from the sequence of a wildtype, full-length hair cell differentiation protein, the two or more deleted amino acids can be contiguous in the sequence of the wildtype, full-length protein. In other examples where two or more amino acids are deleted from the sequence of a wildtype, full-length hair cell differentiation protein, the two or more deleted amino acids are not contiguous in the sequence of the wildtype, full-length protein. One skilled in the art would appreciate that amino acids that are not conserved between wildtype, full-length hair cell differentiation proteins from different species can be deleted without losing activity, while those amino acids that are conserved between wildtype, full-length hair cell differentiation proteins from different species should not be deleted as they are more likely (than amino acids that are not conserved between different species) to be involved in activity.

In some examples, an active hair cell differentiation protein can, e.g., include a sequence of a wildtype, full-length hair cell differentiation protein that has between 1 amino acid to about 100 amino acids, 1 amino acid to about 95 amino acids, 1 amino acid to about 90 amino acids, 1 amino acid to about 85 amino acids, 1 amino acid to about 80 amino acids, 1 amino acid to about 75 amino acids, 1 amino acid to about 70 amino acids, 1 amino acid to about 65 amino acids, 1 amino acid to about 60 amino acids, 1 amino acid to about 55 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 9 amino acids, 1 amino acid to about 8 amino acids, 1 amino acid to about 7 amino acids, 1 amino acid to about 6 amino acids, 1 amino acid to about 5 amino acids, 1 amino acid to about 4 amino acids, 1 amino acid to about 3 amino acids, about 2 amino acids to about 100 amino acids, about 2 amino acid to about 95 amino acids, about 2 amino acids to about 90 amino acids, about 2 amino acids to about 85 amino acids, about 2 amino acids to about 80 amino acids, about 2 amino acids to about 75 amino acids, about 2 amino acids to about 70 amino acids, about 2 amino acids to about 65 amino acids, about 2 amino acids to about 60 amino acids, about 2 amino acids to about 55 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids, to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 100 amino acids, about 3 amino acid to about 95 amino acids, about 3 amino acids to about 90 amino acids, about 3 amino acids to about 85 amino acids, about 3 amino acids to about 80 amino acids, about 3 amino acids to about 75 amino acids, about 3 amino acids to about 70 amino acids, about 3 amino acids to about 65 amino acids, about 3 amino acids to about 60 amino acids, about 3 amino acids to about 55 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 100 amino acids, about 4 amino acid to about 95 amino acids, about 4 amino acids to about 90 amino acids, about 4 amino acids to about 85 amino acids, about 4 amino acids to about 80 amino acids, about 4 amino acids to about 75 amino acids, about 4 amino acids to about 70 amino acids, about 4 amino acids to about 65 amino acids, about 4 amino acids to about 60 amino acids, about 4 amino acids to about 55 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 100 amino acids, about 5 amino acid to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 100 amino acids, about 6 amino acid to about 95 amino acids, about 6 amino acids to about 90 amino acids, about 6 amino acids to about 85 amino acids, about 6 amino acids to about 80 amino acids, about 6 amino acids to about 75 amino acids, about 6 amino acids to about 70 amino acids, about 6 amino acids to about 65 amino acids, about 6 amino acids to about 60 amino acids, about 6 amino acids to about 55 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 100 amino acids, about 7 amino acid to about 95 amino acids, about 7 amino acids to about 90 amino acids, about 7 amino acids to about 85 amino acids, about 7 amino acids to about 80 amino acids, about 7 amino acids to about 75 amino acids, about 7 amino acids to about 70 amino acids, about 7 amino acids to about 65 amino acids, about 7 amino acids to about 60 amino acids, about 7 amino acids to about 55 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 100 amino acids, about 8 amino acid to about 95 amino acids, about 8 amino acids to about 90 amino acids, about 8 amino acids to about 85 amino acids, about 8 amino acids to about 80 amino acids, about 8 amino acids to about 75 amino acids, about 8 amino acids to about 70 amino acids, about 8 amino acids to about 65 amino acids, about 8 amino acids to about 60 amino acids, about 8 amino acids to about 55 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 100 amino acids, about 10 amino acid to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 20 amino acids to about 100 amino acids, about 20 amino acid to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 30 amino acids to about 100 amino acids, about 30 amino acid to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 40 amino acids to about 100 amino acids, about 40 amino acid to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 50 amino acids to about 100 amino acids, about 50 amino acid to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 60 amino acids to about 100 amino acids, about 60 amino acid to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 70 amino acids to about 100 amino acids, about 70 amino acid to about 95 amino acids, about 70 amino acids to about 90 amino acids, about 70 amino acids to about 85 amino acids, about 70 amino acids to about 80 amino acids, about 70 amino acids to about 75 amino acids, about 80 amino acids to about 100 amino acids, about 80 amino acid to about 95 amino acids, about 80 amino acids to about 90 amino acids, about 80 amino acids to about 85 amino acids, about 90 amino acids to about 100 amino acids, about 90 amino acids to about 95 amino acids, or about 95 amino acids to about 100 amino acids, removed from its N-terminus and/or 1 amino acid to 100 amino acids (or any of the subranges of this range described herein) removed from its C-terminus.

In some embodiments, an active hair cell differentiation protein can, e.g., include the sequence of a wildtype, full-length hair cell differentiation protein where 1 amino acid to 50 amino acids, 1 amino acid to 45 amino acids, 1 amino acid to 40 amino acids, 1 amino acid to 35 amino acids, 1 amino acid to 30 amino acids, 1 amino acid to 25 amino acids, 1 amino acid to 20 amino acids, 1 amino acid to 15 amino acids, 1 amino acid to 10 amino acids, 1 amino acid to 9 amino acids, 1 amino acid to 8 amino acids, 1 amino acid to 7 amino acids, 1 amino acid to 6 amino acids, 1 amino acid to 5 amino acids, 1 amino acid to 4 amino acids, 1 amino acid to 3 amino acids, about 2 amino acids to 50 amino acids, about 2 amino acids to 45 amino acids, about 2 amino acids to 40 amino acids, about 2 amino acids to 35 amino acids, about 2 amino acids to 30 amino acids, about 2 amino acids to 25 amino acids, about 2 amino acids to 20 amino acids, about 2 amino acids to 15 amino acids, about 2 amino acids to 10 amino acids, about 2 amino acids to 9 amino acids, about 2 amino acids to 8 amino acids, about 2 amino acids to 7 amino acids, about 2 amino acids to 6 amino acids, about 2 amino acids to 5 amino acids, about 2 amino acids to 4 amino acids, about 3 amino acids to 50 amino acids, about 3 amino acids to 45 amino acids, about 3 amino acids to 40 amino acids, about 3 amino acids to 35 amino acids, about 3 amino acids to 30 amino acids, about 3 amino acids to 25 amino acids, about 3 amino acids to 20 amino acids, about 3 amino acids to 15 amino acids, about 3 amino acids to 10 amino acids, about 3 amino acids to 9 amino acids, about 3 amino acids to 8 amino acids, about 3 amino acids to 7 amino acids, about 3 amino acids to 6 amino acids, about 3 amino acids to 5 amino acids, about 4 amino acids to 50 amino acids, about 4 amino acids to 45 amino acids, about 4 amino acids to 40 amino acids, about 4 amino acids to 35 amino acids, about 4 amino acids to 30 amino acids, about 4 amino acids to 25 amino acids, about 4 amino acids to 20 amino acids, about 4 amino acids to 15 amino acids, about 4 amino acids to 10 amino acids, about 4 amino acids to 9 amino acids, about 4 amino acids to 8 amino acids, about 4 amino acids to 7 amino acids, about 4 amino acids to 6 amino acids, about 5 amino acids to 50 amino acids, about 5 amino acids to 45 amino acids, about 5 amino acids to 40 amino acids, about 5 amino acids to 35 amino acids, about 5 amino acids to 30 amino acids, about 5 amino acids to 25 amino acids, about 5 amino acids to 20 amino acids, about 5 amino acids to 15 amino acids, about 5 amino acids to 10 amino acids, about 5 amino acids to 9 amino acids, about 5 amino acids to 8 amino acids, about 5 amino acids to 7 amino acids, about 6 amino acids to 50 amino acids, about 6 amino acids to 45 amino acids, about 6 amino acids to 40 amino acids, about 6 amino acids to 35 amino acids, about 6 amino acids to 30 amino acids, about 6 amino acids to 25 amino acids, about 6 amino acids to 20 amino acids, about 6 amino acids to 15 amino acids, about 6 amino acids to 10 amino acids, about 6 amino acids to 9 amino acids, about 6 amino acids to 8 amino acids, about 7 amino acids to 50 amino acids, about 7 amino acids to 45 amino acids, about 7 amino acids to 40 amino acids, about 7 amino acids to 35 amino acids, about 7 amino acids to 30 amino acids, about 7 amino acids to 25 amino acids, about 7 amino acids to 20 amino acids, about 7 amino acids to 15 amino acids, about 7 amino acids to 10 amino acids, about 7 amino acids to 9 amino acids, about 8 amino acids to 50 amino acids, about 8 amino acids to 45 amino acids, about 8 amino acids to 40 amino acids, about 8 amino acids to 35 amino acids, about 8 amino acids to 30 amino acids, about 8 amino acids to 25 amino acids, about 8 amino acids to 20 amino acids, about 8 amino acids to 15 amino acids, about 8 amino acids to 10 amino acids, about 10 amino acids to 50 amino acids, about 10 amino acids to 45 amino acids, about 10 amino acids to 40 amino acids, about 10 amino acids to 35 amino acids, about 10 amino acids to 30 amino acids, about 10 amino acids to 25 amino acids, about 10 amino acids to 20 amino acids, about 10 amino acids to 15 amino acids, about 15 amino acids to 50 amino acids, about 15 amino acids to 45 amino acids, about 15 amino acids to 40 amino acids, about 15 amino acids to 35 amino acids, about 15 amino acids to 30 amino acids, about 15 amino acids to 25 amino acids, about 15 amino acids to 20 amino acids, about 20 amino acids to 50 amino acids, about 20 amino acids to 45 amino acids, about 20 amino acids to 40 amino acids, about 20 amino acids to 35 amino acids, about 20 amino acids to 30 amino acids, about 20 amino acids to 25 amino acids, about 25 amino acids to 50 amino acids, about 25 amino acids to 45 amino acids, about 25 amino acids to 40 amino acids, about 25 amino acids to 35 amino acids, about 25 amino acids to 30 amino acids, about 30 amino acids to 50 amino acids, about 30 amino acids to 45 amino acids, about 30 amino acids to 40 amino acids, about 30 amino acids to 35 amino acids, about 35 amino acids to 50 amino acids, about 35 amino acids to 45 amino acids, about 35 amino acids to 40 amino acids, about 40 amino acids to 50 amino acids, about 40 amino acids to 45 amino acids, or about 45 amino acids to about 50 amino acids, are inserted. In some examples, the 1 amino acid to 50 amino acids (or any subrange thereof) can be inserted as a contiguous sequence into the sequence of a wildtype, full-length protein. In some examples, the 1 amino acid to 50 amino acids (or any subrange thereof) are not inserted as a contiguous sequence into the sequence of a wildtype, full-length protein. As can be appreciated in the art, the 1 amino acid to 50 amino acids can be inserted into a portion of the sequence of a wildtype, full-length protein that is not well-conserved between species.

Atonal Basic Helix-Loop-Helix Transcription Factor 1 (Atoh1)

The ATOH1 gene encodes atonal basic helix-loop-helix (bHLH) transcription factor 1. ATOH1 is a primary regulator of hair cell differentiation (Kawamoto et al., J. Neurosci. (2003) 23(11):4395-4400; Izumikawa et al. (2005) Nat. Med. 11(3): 271-276; Minoda et al. (2007) Hear Res. 232(1-2): 44-51; Atkinson et al. (2014) PLoS One 9(7): e102077; Kuo et al. (2015) J. Neurosci 35(30): 10786-10798; Walters et al. (2017) Cell Rep 19(2): 307-320).

The human ATOH1 gene is located on chromosome 4q22. It contains 1 exon encompassing ˜2 kilobases (kb) (NCBI Accession No. NM_005172.1). The full-length wildtype ATOH1 protein expressed from the human ATOH1 gene is 354 amino acids in length.

Non-limiting examples of detecting techniques include: real-time polymerase chain reaction (RT-PCR), PCR, sequencing, Southern blotting, and Northern blotting.

An exemplary human wildtype ATOH1 protein is or includes the sequence of SEQ ID NO: 1. Non-limiting examples of a nucleic acid encoding a wildtype ATOH1 protein is or includes SEQ ID NO: 4. As can be appreciated in the art, at least some or all of the codons in SEQ ID NO: 4 can be codon-optimized to allow for optimal expression in a non-human primate.

Human Full-length Wildtype ATOH1 Protein
(SEQ ID NO: 1)
MSRLLHAEEWAEVKELGDHHRQPQPHHLPQPPPPPQPPATLQAREHPVYP
PELSLLDSTDPRAWLAPTLQGICTARAAQYLLHSPELGASEAAAPRDEVD
GRGELVRRSSGGASSSKSPGPVKVREQLCKLKGGVVVDELGCSRQRAPSS
KQVNGVQKQRRLAANARERRRMHGLNHAFDQLRNVIPSFNNDKKLSKYET
LQMAQIYINALSELLQTPSGGEQPPPPPASCKSDHHHLRTAASYEGGAGN
ATAAGAQQASGGSQRPTPPGSCRTRFSAPASAGGYSVQLDALHFSTFEDS
ALTAMMAQKNLSPSLPGSILQPVQEENSKTSPRSHRSDGEFSPHSHYSDS
DEAS
Mouse Full-Length Wildtype ATOH1 Protein
(SEQ ID NO: 2)
MSRLLHAEEWAEVKELGDHHRHPQPHHVPPLTPQPPATLQARDLPVYPAE
LSLLDSTDPRAWLTPTLQGLCTARAAQYLLHSPELGASEAAAPRDEADSQ
GELVRRSGCGGLSKSPGPVKVREQLCKLKGGVVVDELGCSRQRAPSSKQV
NGVQKQRRLAANARERRRMHGLNHAFDQLRNVIPSFNNDKKLSKYETLQM
AQIYINALSELLQTPNVGEQPPPPTASCKNDHHHLRTASSYEGGAGASAV
AGAQPAPGGGPRPTPPGPCRTRFSGPASSGGYSVQLDALHFPAFEDRALT
AMMAQKDLSPSLPGGILQPVQEDNSKTSPRSHRSDGEFSPHSHYSDSDEA
S
Rat Full-Length Wildtype ATOH1 Protein
(SEQ ID NO: 3)
MSRLLHAEEWAEVKELGDHHRHPQPHHIPQLTPQPPATLQARDHPVYPAE
LSLLDSTDPRAWLTPTLQGLCTARAAQYLLHSPELGASEAAAPGDEADGQ
GELVRRSGCGGLSKSPGPVKVREQLCKLKGGVVVDELGCSRQRAPSSKQV
NGVQKQRRLAANARERRRMHGLNHAFDQLRNVIPSFNNDKKLSKYETLQM
AQIYINALSELLQTPSVGEQPPPPAASCKNDHHHLRAAASYEGGAGASAV
AGAQPAPGGGPRPTPPGACRTRFSTPASSGGYSVQLDALHFPAFEDRALT
AMMAQKDLSPSLPGGILQPVPEDSSKTSPRSHRSDGEFSPHSHYSDSDEA
S
Human Wildtype ATOH1 cDNA
(SEQ ID NO: 4)
atgtcccgcctgctgcatgcagaagagtgggctgaagtgaaggagttggg
agaccaccatcgccagccccagccgcatcatctcccgcaaccgccgccgc
cgccgcagccacctgcaactttgcaggcgagagagcatcccgtctacccg
cctgagctgtccctcctggacagcaccgacccacgcgcctggctggctcc
cactttgcagggcatctgcacggcacgcgccgcccagtatttgctacatt
ccccggagctgggtgcctcagaggccgctgcgccccgggacgaggtggac
ggccggggggagctggtaaggaggagcagcggcggtgccagcagcagcaa
gagccccgggccggtgaaagtgcgggaacagctgtgcaagctgaaaggcg
gggtggtggtagacgagctgggctgcagccgccaacgggccccttccagc
aaacaggtgaatggggtgcagaagcagagacggctagcagccaacgccag
ggagcggcgcaggatgcatgggctgaaccacgccttcgaccagctgcgca
atgttatcccgtcgttcaacaacgacaagaagctgtccaaatatgagacc
ctgcagatggcccaaatctacatcaacgccttgtccgagctgctacaaac
gcccagcggaggggaacagccaccgccgcctccagcctcctgcaaaagcg
accaccaccaccttcgcaccgcggcctcctatgaagggggcgcgggcaac
gcgaccgcagctggggctcagcaggcttccggagggagccagcggccgac
cccgcccgggagttgccggactcgcttctcagccccagcttctgcgggag
ggtactcggtgcagctggacgctctgcacttctcgactttcgaggacagc
gccctgacagcgatgatggcgcaaaagaatttgtctccttctctccccgg
gagcatcttgcagccagtgcaggaggaaaacagcaaaacttcgcctcggt
cccacagaagcgacggggaattttccccccattcccattacagtgactcg
gatgaggcaagttag

A non-limiting example of a human wildtype ATOH1 genomic DNA sequence is SEQ ID NO: 5. The exon in SEQ ID NO: 5 is: nucleotide positions 1-1065 (exon 1).

Human Wildtype ATOH1 Gene
(SEQ ID NO: 5)

| | |
|---|---|
| 1 | atgtcccgcc tgctgcatgc agaagagtgg gctgaagtga |
| | aggagttggg agaccaccat |
| 61 | cgccagcccc agccgcatca tctcccgcaa ccgccgccgc |
| | cgccgcagcc acctgcaact |
| 121 | ttgcaggcga gagagcatcc cgtctacccg cctgagctgt |
| | ccctcctgga cagcaccgac |
| 181 | ccacgcgcct ggctggctcc cactttgcag ggcatctgca |
| | cggcacgcgc cgcccagtat |
| 241 | ttgctacatt ccccggagct gggtgcctca gaggccgctg |
| | cgccccggga cgaggtggac |
| 301 | ggccgggggg agctggtaag gaggagcagc ggcggtgcca |
| | gcagcagcaa gagccccggg |
| 361 | ccggtgaaag tgcgggaaca gctgtgcaag ctgaaaggcg |
| | gggtggtggt agacgagctg |
| 421 | ggctgcagcc gccaacgggc cccttccagc aaacaggtga |
| | atggggtgca gaagcagaga |
| 481 | cggctagcag ccaacgccag ggagcggcgc aggatgcatg |
| | ggctgaacca cgccttcgac |
| 541 | cagctgcgca atgttatccc gtcgttcaac aacgacaaga |
| | agctgtccaa atatgagacc |
| 601 | ctgcagatgg cccaaatcta catcaacgcc ttgtccgagc |
| | tgctacaaac gcccagcgga |
| 661 | ggggaacagc caccgccgcc tccagcctcc tgcaaaagcg |
| | accaccacca ccttcgcacc |
| 721 | gcggcctcct atgaaggggg cgcgggcaac gcgaccgcag |
| | ctggggctca gcaggcttcc |
| 781 | ggagggagcc agcggccgac cccgcccggg agttgccgga |
| | ctcgcttctc agccccagct |
| 841 | tctgcgggag ggtactcggt gcagctggac gctctgcact |
| | tctcgacttt cgaggacagc |
| 901 | gccctgacag cgatgatggc gcaaaagaat ttgtctcctt |
| | ctctccccgg gagcatcttg |
| 961 | cagccagtgc aggaggaaaa cagcaaaact tcgcctcggt |
| | cccacagaag cgacggggaa |
| 1021 | ttttcccccc attcccatta cagtgactcg gatgaggcaa |
| | gttag |

POU Class 4 Homeobox 3 (Pou4f3)

The POU4F3 gene encodes POU class 4 homeobox 3, and acts as a transcriptional activator. POU4F3 activates ATOH1 transcription in early development and is later further activated by ATOH1 and required for hair cell survival after birth. POU4F3 activates NT3 and BDNF. Mutations in POU4F3 have been associated with hearing loss (Lee et al. (2010) Biochem Biophys Res Commun 396(3):626-630; Clough et al. (2004) Biochem Biophys Res Commun 324(1):372-381; Costa et al. (2015) Development 142(11):1948-1959; and Walters et al. (2017) Cell Rep 19(2):307-320).

The human POU4F3 gene is located on chromosome 5q32. It contains 2 exons encompassing ˜15 kilobases (kb) (NCBI Accession No. NG_011885.1). The full-length wildtype POU4F3 protein expressed from the human POU4F3 gene is 338 amino acids in length.

Various mutations in the POU4F3 gene have been associated with hearing loss due to hair cell degeneration. For example, a nonsense mutation c.337C>T in POU4F3 was identified to cause autosomal dominant hearing loss (Zhang et al. (2016) Neural Plast doi:10.1155/2016/1512831).

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype POU4F3 protein is or includes the sequence of SEQ ID NO: 6. Non-limiting examples of nucleic acid encoding a wildtype POU4F3 protein are or include SEQ ID NO: 9. As can be appreciated in the art, at least some or all of the codons in SEQ ID NO: 9 can be codon-optimized to allow for optimal expression in a non-human primate.

Human Full-length Wildtype POU4F3 Protein
(SEQ ID NO: 6)
MMAMNSKQPFGMHPVLQEPKFSSLHSGSEAMRRVCLPAPQLQGNIFGSFD
ESLLARAEALAAVDIVSHGKNHPFKPDATYHTMSSVPCTSTSSTVPISHP
AALTSHPHHAVHQGLEGDLLEHISPTLSVSGLGAPEHSVMPAQIHPHHLG
AMGHLHQAMGMSHPHTVAPHSAMPACLSDVESDPRELEAFAERFKQRRIK
LGVTQADVGAALANLKIPGVGSLSQSTICRFESLTLSHNNMIALKPVLQA
WLEEAEAAYREKNSKPELFNGSERKRKRTSIAAPEKRSLEAYFAIQPRPS
SEKIAAIAEKLDLKKNVVRVWFCNQRQKQKRMKYSAVH
Mouse Full-Length Wildtype POU4F3 Protein
(SEQ ID NO: 7)
MMAMNAKQPFGMHPVLQEPKFSSLHSGSEAMRRVCLPAPQLQGNIFGSFD
ESLLARAEALAAVDIVSHGKNHPFKPDATYHTMSSVPCTSTSPTVPISHP
AALTSHPHHAVHQGLEGDLLEHISPTLSVSGLGAPEHSVMPAQIHPHHLG
AMGHLHQAMGMSHPHAVAPHSAMPACLSDVESDPRELEAFAERFKQRRIK
LGVTQADVGAALANLKIPGVGSLSQSTICRFESLTLSHNNMIALKPVLQA
WLEEAEAAYREKNSKPELFNGSERKRKRTSIAAPEKRSLEAYFAIQPRPS
SEKIAAIAEKLDLKKNVVRVWFCNQRQKQKRMKYSAVH
Rat Full-Length Wildtype POU4F3 Protein
(SEQ ID NO: 8)
MMAMNAKQPFGMHPVLQEPKFSSLHSGSEAMRRVCLPAPQLQGNIFGSFD
ESLLARAEALAAVDIVSHGKNHPFKPDATYHTMSSVPCTSTSPTVPISHP
AALTSHPHHPVHQGLEGDLLEHISPTLSVSGLGAPEHSVMPAQIHPHHLG
AMGHLHQAMGMSHPHAVAPHSAMPACLSDVESDPRELEAFAERFKQRRIK
LGVTQADVGAALANLKIPGVGSLSQSTICRFESLTLSHNNMIALKPVLQA
WLEEAEAAYREKNSKPELFNGSERKRKRTSIAAPEKRSLEAYFAIQPRPS
SEKIAAIAEKLDLKKNVVRVWFCNQRQKQKRMKYSAVH
Human Wildtype POU4F3 cDNA
(SEQ ID NO: 9)
atgatggccatgaactccaagcagcctttcggcatgcacccggtgctgca
agaacccaaattctccagtctgcactctggctccgaggccatgcgccgag
tctgtctcccagccccgcagctgcagggtaatatatttggaagctttgat
gagagcctgctggcacgcgccgaagctctggcggcggtggatatcgtctc
ccacggcaagaaccatccgttcaagcccgacgccacctaccataccatga
gcagcgtgccctgcacgtccacttcgtccaccgtgcccatctcccaccca
gctgcgctcacctcacaccctcaccacgccgtgcaccagggcctcgaagg
cgacctgctggagcacatctcgcccacgctgagtgtgagcggcctgggcg
ctccggaacactcggtgatgcccgcacagatccatccacaccacctgggc
gccatgggccacctgcaccaggccatgggcatgagtcacccgcacaccgt
ggcccctcatagcgccatgcctgcatgcctcagcgacgtggagtcagacc
cgcgcgagctggaagccttcgccgagcgcttcaagcagcggcgcatcaag
ctgggggtgacccaggcggacgtgggcgcggctctggctaatctcaagat
ccccggcgtgggctcgctgagccaaagcaccatctgcaggttcgagtctc
tcactctctcgcacaacaacatgatcgctctcaagccggtgctccaggcc
tggttggaggaggccgaggccgcctaccgagagaag aacagcaagccag
agctcttcaacggcagcgaacggaagcgcaaacgcacgtccatcgcggcg
ccggagaagcgttcactcgaggcctatttcgctatccagccacgtccttc
atctgagaagatcgcggccatcgctgagaaactggaccttaaaaagaacg
tggtgagagtctggttctgcaaccagagacagaaacagaaacgaatgaag
tattcggctgtccactga

A non-limiting example of a human wildtype POU4F3 genomic DNA sequence is SEQ ID NO: 10. The exons in SEQ ID NO: 10 are: nucleotide positions 1-209 (exon 1) and nucleotide positions 525-1497 (exon 2). The intron in SEQ ID NO: 10 is: nucleotide positions 210-524 (intron 1).

| Human Wildtype POU4F3 Gene | |
|---|---|
| (SEQ ID NO: 10) | |
| 1 | cgctgagcag cgctcacttg gagagcggca agcaagctag acaagcctga ttccatgtca |
| 61 | cccgctgcca ccctgccagg agcgcgaaga tgatggccat caactccaag cagcctttcg |
| 121 | gcatgcaccc ggtgctgcaa gaacccaaat tctccagtct gcactctggc tccgaggcca |
| 181 | tgcgccgagt ctgtctccca gccccgcagg tacgtagtgg agcataatta ccgctctaag |
| 241 | gcacattttt tgacaggcac tagcttcatg tttttttcat gtcgcccaga acaatcgccg |
| 301 | ctgtctgaac ccctctcctt gtctcccccg cgttctctcc cggcgcgctc tctctctcat |
| 361 | tcatgtctct gatccacacg tctgttccag cagagccgct gcctccgtat taatttttat |
| 421 | gacctgggct ttgaggagag gcatctcggt tgcttgaaaa tgtgttttaa tcctgtgttg |
| 481 | acagtattcc ctactgaccg tgctgtgcgc cttctcgctt gcagctgcag ggtaatatat |
| 541 | ttggaagctt tgatgagagc ctgctggcac gcgccgaagc tctggcggcg gtggatatcg |
| 601 | tctcccacgg caagaaccat ccgttcaagc ccgacgccac ctaccatacc atgagcagcg |
| 661 | tgccctgcac gtccacttcg tccaccgtgc ccatctccca cccagctgcg ctcacctcac |
| 721 | accctcacca cgccgtgcac cagggcctcg aaggcgacct gctggagcac atctcgccca |
| 781 | cgctgagtgt gagcggcctg ggcgctccgg aacactcggt gatgcccgca cagatccatc |
| 841 | cacaccacct gggcgccatg ggccacctgc accaggccat gggcatgagt cacccgcaca |
| 901 | ccgtggcccc tcatagcgcc atgcctgcat gcctcagcga cgtggagtca gacccgcgcg |
| 961 | agctggaagc cttcgccgag cgcttcaagc agcggcgcat caagctgggg gtgacccagg |
| 1021 | cggacgtggg cgcggctctg gctaatctca agatccccgg cgtgggctcc ctgagccaaa |
| 1081 | gcaccatctc caggttcgag tctctcactc tctcgcacaa caacatgatc gctctcaagc |
| 1141 | cggtgctcca ggcctggttg gaggaggccg aggccgccta ccgagagaag aacagcaagc |
| 1201 | cagagctctt caacggcagc gaacggaagc gcaaacgcac gtccatcgcg gcgccggaga |
| 1261 | agcgttcact cgaggcctat ttcgctatcc agccacgtcc ttcatctgag aagatcgcgg |
| 1321 | ccatcgctga gaaactggac cttaaaaaga acgtggtgag agtctggttc tgcaaccaga |
| 1381 | gacagaaaca gaaacgaatg aagtattcgg ctgtccactg attgcggcag ggcgcagcgt |
| 1441 | cgggagccgg gagagcctag tgctcatccc tcccgggttc gggggatggt tatcggg |

Catenin Beta 1 (CTNNB1)

The CTNNB1 gene encodes catenin beta 1 (β-Catenin), a protein involved both in transcriptional activation and in adherens junctions. CTNNB1 is required for hair cell development and differentiation. β-Catenin activates ATOH1 through binding to its enhancer. Overexpression or stabilization of CTNNB1 results in supporting cell proliferation and differentiation into hair cells (Shi et al. (2013) Proc Nad Acad Sci USA. 110(34):13851-13856; Kuo et al. (2015) J. Neurosci. 35(30):10786-10798). Knock-out of CTNNB1 in early development prevents hair cell differentiation (Shi et al. (2013) J. Neurosci. 34(19):6470-6479. Overexpression of CTNNB1 induces ectopic hair cells.

The human CTNNB1 gene is located on chromosome 3p22. It contains 15 exons encompassing ˜41 kilobases (kb) (NCBI Accession No. NG_013302.2). The full-length wildtype CTNNB1 protein expressed from the human CTNNB1 gene is 781 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype CTNNB1 protein is or includes the sequence of SEQ ID NO: 11. Non-limiting examples of a nucleic acid encoding a wildtype POU4F3 protein is or includes SEQ ID NO: 14. As can be appreciated in the art, at least some or all of the codons in SEQ ID NO: 14 can be codon-optimized to allow for optimal expression in a non-human primate.

Human Full-length Wildtype CTNNB1 Protein
(SEQ ID NO: 11)
MATQADLMELDMAMEPDRKAAVSHWQQQSYLDSGIHSGATTTAPSLSGKGNPEEEDVDTSQVLYEWEQGFSQSFTQE
QVADIDGQYAMTRAQRVRAAMFPETLDEGMQIPSTQFDAAHPTNVQRLAEPSQMLKHAVVNLINYQDDAELATRAIP
ELTKLLNDEDQVVVNKAAVMVHQLSKKEASRHAIMRSPQMVSAIVRTMQNTNDVETARCTAGTLHNLSHHREGLLAI
FKSGGIPALVKMLGSPVDSVLFYAITTLHNLLLHQEGAKMAVRLAGGLQKMVALLNKTNVKFLAITTDCLQILAYGN
QESKLIILASGGPQALVNIMRTYTYEKLLWTTSRVLKVLSVCSSNKPAIVEAGGMQALGLHLTDPSQRLVQNCLWTL
RNLSDAATKQEGMEGLLGTLVQLLGSDDINVVTCAAGILSNLTCNNYKNKMMVCQVGGIEALVRTVLRAGDREDITE
PAICALRHLTSRHQEAEMAQNAVRLHYGLPVVVKLLHPPSHWPLIKATVGLIRNLALCPANHAPLREQGAIPRLVQL
LVRAHQDTQRRTSMGGTQQQFVEGVRMEEIVEGCTGALHILARDVHNRIVIRGLNTIPLFVQLLYSPIENIQRVAAG
VLCELAQDKEAAEAIEAEGATAPLTELLHSRNEGVATYAAAVLFRMSEDKPQDYKKRLSVELTSSLFRTEPMAWNET
ADLGLDIGAQGEPLGYRQDDPSYRSFHSGGYGQDALGMDPMMEHEMGGHHPGADYPVDGLPDLGHAQDLMDGLPPGD
SNQLAWFDTDL
Mouse Full-length Wildtype CTNNB1 Protein
(SEQ ID NO: 12)
MATQADLMELDMAMEPDRKAAVSHWQQQSYLDSGIHSGATTTAPSLSGKGNPEEEDVDTSQVLYEWEQGFSQSFTQE
QVADIDGQYAMTRAQRVRAAMFPETLDEGMQIPSTQFDAAHPTNVQRLAEPSQMLKHAVVNLINYQDDAELATRAIP
ELTKLLNDEDQVVVNKAAVMVHQLSKKEASRHAIMRSPQMVSAIVRTMQNTNDVETARCTAGTLHNLSHHREGLLAI
FKSGGIPALVKMLGSPVDSVLFYAITTLHNLLLHQEGAKMAVRLAGGLQKMVALLNKTNVKFLAITTDCLQILAYGN
QESKLIILASGGPQALVNIMRTYTYEKLLWTTSRVLKVLSVCSSNKPAIVEAGGMQALGLHLTDPSQRLVQNCLWTL
RNLSDAATKQEGMEGLLGTLVQLLGSDDINVVTCAAGILSNLTCNNYKNKMMVCQVGGIEALVRTVLRAGDREDITE
PAICALRHLTSRHQEAEMAQNAVRLHYGLPVVVKLLHPPSHWPLIKATVGLIRNLALCPANHAPLREQGAIPRLVQL
LVRAHQDTQRRTSMGGTQQQFVEGVRMEEIVEGCTGALHILARDVHNRIVIRGLNTIPLFVQLLYSPIENIQRVAAG
VLCELAQDKEAAEAIEAEGATAPLTELLHSRNEGVATYAAAVLFRMSEDKPQDYKKRLSVELTSSLFRTEPMAWNET
ADLGLDIGAQGEALGYRQDDPSYRSFHSGGYGQDALGMDPMMEHEMGGHHPGADYPVDGLPDLGHAQDLMDGLPPGD
SNQLAWFDTDL
Rat Full-length Wildtype CTNNB1 Protein
(SEQ ID NO: 13)
MATQADLMELDMAMEPDRKAAVSHWQQQSYLDSGIHSGATTTAPSLSGKGNPEEEDVDTSQVLYEWEQGFSQSFTQE
QVADIDGQYAMTRAQRVRAAMFPETLDEGMQIPSTQFDAAHPTNVQRLAEPSQMLKHAVVNLINYQDDAELATRAIP
ELTKLLNDEDQVVVNKAAVMVHQLSKKEASRHAIMRSPQMVSAIVRTMQNTNDVETARCTAGTLHNLSHHREGLLAI
FKSGGIPALVKMLGSPVDSVLFYAITTLHNLLLHQEGAKMAVRLAGGLQKMVALLNKTNVKFLAITTDCLQILAYGN
QESKLIILASGGPQALVNIMRTYTYEKLLWTTSRVLKVLSVCSSNKPAIVEAGGMQALGPHLTDPSQRLVQNCLWTL
RNLSDAATKQEGMEGLLGTLVQLLGSDDINVVTCAAGILSNLTCNNYKNKMMVCQVGGIEALVRTVLRAGDREDITE
PAICALRHLTSRHQEAEMAQNAVRLHYGLPVVVKLLHPPSHWPLIKATVGLIRNLALCPANHAPLREQGAIPRLVQL
LVRAHQDTQRRTSMGGTQQQFVEGVRMEEIVEGCTGALHILARDVHNRIVIRGLNTIPLFVQLLYSPIENIQRVAAG
VLCELAQDKEAAEAIEAEGATAPLTELLHSRNEGVATYAAAVLFRMSEDKPQDYKKRLSVELTSSLFRTEPMAWNET
ADLGLDIGAQGEALGYRQDDPSYRSFHSGGYGQDALGMDPMMEHEMGGHHPGADYPVDGLPDLGHAQDLMDGLPPGD
SNQLAWFDTDL
Human Wildtype CTNNB1 cDNA
(SEQ ID NO: 14)
atggctactcaagctgatttgatggagttggacatggccatggaaccagacagaaaagcggctgttagtcactggca
gcaacagtcttacctggactctggaatccattctggtgccactaccacagctccttctctgagtggtaaaggcaatc
ctgaggaagaggatgtggatacctcccaagtcctgtatgagtgggaacagggattttctcagtccttcactcaagaa
caagtagctgatattgatggacagtatgcaatgactcgagctcagagggtacgagctgctatgttccctgagacatt
agatgagggcatgcagatcccatctacacagtttgatgctgctcatcccactaatgtccagcgtttggctgaaccat
cacagatgctgaaacatgcagttgtaaacttgattaactatcaagatgatgcagaacttgccacacgtgcaatccct
gaactgacaaaactgctaaatgacgaggaccaggtggtggttaataaggctgcagttatggtccatcagctttctaa
aaaggaagcttccagacacgctatcatgcgttctcctcagatggtgtctgctattgtacgtaccatgcagaatacaa
atgatgtagaaacagctcgttgtaccgctgggaccttgcataacctttcccatcatcgtgagggcttactggccatc
tttaagtctggaggcattcctgccctggtgaaaatgcttggttcaccagtggattctgtgttgttttatgccattac
aactctccacaaccttttattacatcaagaaggagctaaaatggcagtgcgtttagctggtgggctgcagaaaatgg
ttgccttgctcaacaaaacaaatgttaaattcttggctattacgacagactgccttcaaattttagcttatggcaac
caagaaagcaagctcatcatactggctagtggtggaccccaagctttagtaaatataatgaggacctatacttacga
aaaactactgtggaccacaagcagagtgctgaaggtgctatctgtctgctctagtaataagccggctattgtagaag
ctggtggaatgcaagctttaggacttcacctgacagatccaagtcaacgtcttgttcagaactgtctttggactctc
aggaatctttcagatgctgcaactaaacaggaagggatggaaggtctccttgggactcttgttcagcttctgggttc
agatgatataaatgtggtcacctgtgcagctggaattctttctaacctcacttgcaataattataagaacaagatga
tggtctgccaagtgggtggtatagaggctcttgtgcgtactgtccttcgggctggtgacagggaagacatcactgag
cctgccatctgtgctcttcgtcatctgaccagccgacaccaagaagcagagatggcccagaatgcagttcgccttca
ctatggactaccagttgtggttaagctcttacacccaccatcccactggcctctgataaaggctactgttggattga
ttcgaaatcttgccctttgtcccgcaaatcatgcacctttgcgtgagcagggtgccattccacgactagttcagttg
cttgttcgtgcacatcaggatacccagcgccgtacgtccatgggtgggacacagcagcaatttgtggagggggtccg
catggaagaaatagttgaaggttgtaccggagcccttcacatcctagctcgggatgttcacaaccgaattgttatca
gaggactaaataccattccattgtttgtgcagctgctttattctcccattgaaaacatccaaagagtagctgcaggg
gtcctctgtgaacttgctcaggacaaggaagctgcagaagctattgaagctgagggagccacagctcctctgacaga
gttacttcactctaggaatgaaggtgtggcgacatatgcagctgctgttttgttccgaatgtctgaggacaagccac
aagattacaagaaacggctttcagttgagctgaccagctctctcttcagaacagagccaatggcttggaatgagact
gctgatcttggacttgatattggtgcccagggagaaccccttggatatcgccaggatgatcctagctatcgttcttt
tcactctggtggatatggccaggatgccttgggtatggaccccatgatggaacatgagatgggtggccaccaccctg
gtgctgactatccagttgatgggctgccagatctggggcatgcccaggacctcatggatgggctgcctccaggtgac
agcaatcagctggcctggtttgatactgacctgtaa

A non-limiting example of a human wildtype CTNNB1 genomic DNA sequence is SEQ ID NO: 15. The exons in SEQ ID NO: 15 are: nucleotide positions 1-220 (exon 1), nucleotide positions 24571-24631 (exon 2), nucleotide positions 25076-25303 (exon 3), nucleotide positions 25504-25757 (exon 4), nucleotide positions 25884-26122 (exon 5), nucleotide positions 26210-26411 (exon 6), nucleotide positions 27758-27902 (exon 7), nucleotide positions 33891-33994 (exon 8), nucleotide positions 34079-34417 (exon 9), nucleotide positions 34689-34847 (exon 10), nucleotide positions 36274-36393 (exon 11), nucleotide positions 36899-37049 (exon 12), nucleotide positions 37138-37259 (exon 13), nucleotide positions 38566-38626 (exon 14), and nucleotide positions 39684-40998 (exon 15). The introns in SEQ ID NO: 15 are: nucleotide positions 221-24570 (intron 1), nucleotide positions 24632-25075 (intron 2), nucleotide positions 25304-25503 (intron 3), nucleotide positions 25758-24883 (intron 4), nucleotide positions 26123-26209 (intron 5), nucleotide positions 26412-27757 (intron 6), nucleotide positions 27903-33890 (intron 7), nucleotide positions 33995-34078 (intron 8), nucleotide positions 34418-34688 (intron 9), nucleotide positions 34848-36273 (intron 10), nucleotide positions 36394-36898 (intron 11), nucleotide positions 37050-37137 (intron 12), nucleotide position 37260-38565 (intron 13), and nucleotide position 38627-39683 (intron 14).

| Human Wildtype CTNNB1 Gene | |
|---|---|
| (SEQ ID NO: 15) | |
| 1 | aggatacagc ggcttctgcg cgacttataa gagctccttg tgcggcgcca ttttaagcct |
| 61 | ctcggtctgt ggcagcagcg ttggcccggc cccgggagcg gagagcgagg ggaggcggag |
| 121 | acggaggaag gtctgaggag cagcttcagt ccccgccgag ccgccaccgc aggtcgagga |
| 181 | cggtcggact cccgcggcgg gaggagcctg ttcccctgag gtgcttgggc gctcctttcc |
| 241 | ttatccttcc ggggctgctc ccgcttcctc tcggagccaa acttcgtagc aggcgcgcgg |
| 301 | tccgggcggc gggctgggcg cagccgggag gcctggggtt gggagcgggg agctcaggtg |
| 361 | ggggacggtg agggtgggcc gcgcccgggg cgcggagggc ggcggccggg cccgggttcc |
| 421 | ggtcgcgctg cctctctggg gccctggggg catcgcttgc ggggaggggg cgccgcgggg |
| 481 | gcgcgtacag gagcccggat ggcaggcggg gtgggggtgg gggtgggggt ctgtggtttc |
| 541 | cgtccggggc tctggccttg gccgagtttg ggggagggac ccggtgcctc gggatgcgcc |
| 601 | gggccctggg tggggggcgg ggtggggacg gggggctccg ccttctcagc tcttgcggcg |
| 661 | agttggggtt cgggcgctga ggcagagacg ccaccctaag tcccatcagt cctggggatc |
| 721 | ggaccagtgg actttctctt aagatttcct ctttcattct taagaataga agtgttatta |
| 781 | ttttttttaa tgccctggct atgtgagttt gaatcgaagc aactttaaac cttagagcaa |
| 841 | ctaaactcta agtgcagcgg gtgcgatgcg tcagtagggt gagcacataa aaaatccatg |
| 901 | tcttgcacct gtattttagc gtactatgca ggtgagtgaa agcagtggat aatgtactgg |
| 961 | gagtcttatg gatttatggt agtgggtatg agaccctggt gaaataaggg ggtggaggaa |
| 1021 | ggcgaaggtg atggcttact gtttcttacc aagtgaactg caggattcag cctctgactc |
| 1081 | agaccgcttc gagaattttg ttcgtagaaa taatttaaat ttattcaaat agtttgatgg |
| 1141 | cagctaaaat tgaattatag agcacgtttt cttttcagcg gagtgaattt ttccttcgct |
| 1201 | ccaaagctgg ccaaatggaa ttcaagcatt gcaacttctt tcagtgtttt gtctggagag |
| 1261 | aggactttga accgagactt ttcgaagtta agttcctata gcctgcttct gaatctgcca |
| 1321 | agcttgaaag ctttggcagt tgggtgtatg tagttgttgc cttcgttctc ttcccttttg |
| 1381 | gagggagcgt tgtctcctac tttgtatctt ccagacatct gtggtcttcc ccccacccct |
| 1441 | cgagtttgtg agtggtgaat gaagaaagac taggctgctg gtatgcagag gtcggcaaaa |
| 1501 | ggaaatcgag gagtggtttt agtgaaatga gagctttgta tcatgaataa tggtggctta |
| 1561 | ggctagacat caacttgaag agacggcagc atttcctttc ataaagtcta ggctaatgtt |
| 1621 | tttcagatcg ctaagttgta gtttgtctgg aatttaggaa gccatttcag tatttgtcac |
| 1681 | ttggtgaacg aacattcaat accttcagat gtcttcgtgt tgacttgtat tcatcctaag |
| 1741 | aaatagtaaa tatagtctca agtgttattt atgttatact gctggtttat tctctgctta |
| 1801 | aattattgac ataaatttct actttggagg cttttcgttt gaactaaggc tgtgcggaat |
| 1861 | ttattttact tttatattta aatctttgaa aaatctctga ttaaaaaaaa agtaccctta |
| 1921 | aaggtttgag gatgtccttt cacaccagac aaaatttggt taatttgcgc ccaatattca |
| 1981 | ttactttgac ctaacctttg ttctgaaggc cgtgtacaag gacaaggccc tgagattatt |
| 2041 | gcaacagtaa cttgaaaaac tttcagaagt ctattctgta ggattaaagg aatgctgaga |
| 2101 | ctattcaagt ttgaagtcct gggggtgggg aaaaataaaa aacctgtgct agaaagctta |
| 2161 | gtatagcatg taactttaga gtcctgtgga gtcctgagtc tcccacagac cagaacagtc |
| 2221 | atttaaaagt tttcaggaaa aaccaactta aaaaaaaata aggtggctaa ttaaaaaaaa |
| 2281 | atgaagcatt taacagtgtt caggtttcag agtatggaag aggggttttt taaactgtta |
| 2341 | tctgattatt tcttttacca acatgatata gaaaagtgta tttccagtat taaaatttat |
| 2401 | cagactgagc ttactgttcc tgttaatgac tggaataaaa attggcataa atgagggtct |
| 2461 | gtatgcttgt tttaataaca ccaccaccaa gatagaaaac gaggaggcaa gtttctccaa |
| 2521 | gggtattttg aaatgtgtta gcaaaactat tgcagatact cgtttttgtt atagggtgag |
| 2581 | gtggggagag gcgcatgcta agtattgttg aaactaggga tgtagagaat taaaagtttg |
| 2641 | aatataatta ttttgtagtt ataagtagca gtgaaattaa atctcctgca atagactata |
| 2701 | gaagtatatt tagccaaatg aaacttcagt gttattgaaa tgaaataata catctgtcct |
| 2761 | gttacaagat tatttttatt tctcttgtgg tttcctagct tctgataatc aataattgta |
| 2821 | gatgagtagg tggtaagttt taagtttgta ctttgagctt agtcggaagc atgcttgact |
| 2881 | gccaacccgg ggcacaaagg atgaaggctt ttagaactgg acaaacttct aacaaaaggt |
| 2941 | atttgcaact cttttgtagt gtgtcatgtt gatttgtgac attgtttttg aaaatatgtg |
| 3001 | ttaacttagt tttcttgtag ccctcttttt attggaactg tggtatctat tgttgaaact |
| 3061 | gcttgactga gaacattttt ataccataaa agtaaatagt aaacatagcc caggagcggc |
| 3121 | ttctggtttg tccatcgtat gtagccattg cctccttgta ctctcattga gaagatactg |
| 3181 | atttgcagat tcagttgtcc ttctctaaca gactatttat gtaatattgc agttgtgatt |
| 3241 | gtgataggta agtggaccag tcggttaaaa taaatactca ggtttcacaa aaggaaaata |
| 3301 | atatgatttg tgttgatcta aatgagtata ggagttaact cctatagttt ttcatcactt |
| 3361 | aaactcaggg gaaagttctt tatttcctct gtttacttaa gaatgctgct tttgtgtttc |
| 3421 | atgcaagact gagcttgact cagtttgaaa cctaggctca tctgttgagg cctgaaccct |
| 3481 | gctgtccttg aagtatgcat ataatttgct tccttcctaa ggaaaaataa gctcttgaaa |
| 3541 | gataaagtca atcacattag gaacccattt ttagggttta gccacttttt tttttttttt |
| 3601 | tttttaactc atgggcatct cttctgttaa gagacattcc ccactctcca agtttccctc |
| 3661 | aagcctgaag cagcagagtg agtagtgttg gagcatgttt tcattgcatg cttgggtcat |
| 3721 | gttgagtgcc ctccagtgga tatagtataa tgcttgtgat tttttttttt ttaattccaa |
| 3781 | acaagtttat gtgggatata tttaggaata gttctgatga gggagaatca actaagaaac |
| 3841 | ctttgatttc taaaataatt aatatcatta ctgctaatta aaatacaggc ttgagaaaat |
| 3901 | gtcttctcag ccaatatttg cagtagaaaa gtcgggaggt tttttaaggt cactttgagt |
| 3961 | aggcagttct gcttaaatat atcataatga taaaccagaa tctcagtata gtactttagg |
| 4021 | aggtaaaaga tcataatatt cagttatatt gatgaattac agcaactgaa attctcagaa |
| 4081 | aaaaattaat gaaaatgtga attgtcaatt tgtctaaaat cattcacaga gtaaaacata |
| 4141 | agtgctcaac ttgattatat taggaaatag atagaaataa aggtaattga gccagtgtat |
| 4201 | gtgacctaaa atataatgcc cttagtgacc atagggttgg tctcatttgt acatagtggt |
| 4261 | gggccatgat gaactgtgtt ttgccctttg aatttttcct taaaaagctt tctctaggct |
| 4321 | cctatgttca tggtttttct gttagtaata ttattttctg aaaatccatg tttcaaatca |
| 4381 | gaatctaatt agcaacagga atgaagctta ttctaaatta gtttttggaa gttaaacggt |
| 4441 | cagcatatgg aaatttttca gggtttagat ttttaaaaat ttgtttttca gaatatgttg |
| 4501 | ctggaatgaa aacgttagcg tagggacgga aaatgacact taccagtgat tgctttactt |
| 4561 | tgcctgtgga attcagtgta attttgtgga aacattggta tatgattttt tactacttaa |
| 4621 | gaaatgtatt gctatagtta gggttttttt ttttttaaag gcaagaatgc ctcaagtgct |
| 4681 | ttatgtgaat gattatttca ggatggatta aatattcctc catcaaggac catacttgta |
| 4741 | aatcagtgat ttccaagttg gtgcttagta tttacagcat ttactgtcta taagcttctg |
| 4801 | ttctgatttt tcaagagttt tctgagaaat gagagtaggc ttaaaagttc tttgaaaaat |
| 4861 | tatgtacata caacttactg aaaaaaattg ctaccgggga cttaatttgt ctcttgaaat |
| 4921 | gggctacttg ccttcattaa tgtagcatac tacaatttga tgttcaagat atgttactaa |
| 4981 | gaataagatc gctttcagaa gccttatata ggattggtct tactacattg tagtgggaat |
| 5041 | ggctactcaa atgtctccag ggccagttag gtattgggta aatgggacca tgcagactat |
| 5101 | taaaaattga agtgcacatg aagcagccag tcataagcag ctccagccac tgtgtgggaa |
| 5161 | tatagtttat gttgccagat catctgattt ctttccccta agtgggaaat ccagatcaat |
| 5221 | gtacatctct tgatttgcaa gtgttggtga acaaaattca tattttaaga tgctgtattc |
| 5281 | agcacaaatt aaatacactt atttgctgaa tactgccagt ttgtccctct gcagtagtac |
| 5341 | catttgaagt acagtgtttt cataatgatt ctgtgaaatg actggttctg tgaatgtaca |
| 5401 | taatttagca gataacattg ttaaattatt aggtttgtat ttatttaggc acttgggaaa |
| 5461 | tgccttgtgt caattgatta tagattagga gcttaaaagc aagatttata ttatcaactt |
| 5521 | atttgtgaag actgggaaac ccacattttt aaagttagga attaagatgg ccaggttcaa |
| 5581 | ggaaaagggg gagaagtaac tttcttatta ctcaaccatc ttaaatagag ttctttaagt |
| 5641 | gtatttttaa gaggtctcaa aacttaatct gaagggacgt caaatgctgg acaaattctg |
| 5701 | tgtatacaac tcaagtcagc ccccaatttt actggtcttt aaatcatgtc ctttttacca |
| 5761 | gaagtttgca tttctaagct aaactattac tgttagacta gatccaaaac ttaaaaacag |
| 5821 | tttaggtaat taaaaattaa ttgaatataa acgttttact taaattaatg gcaaatggct |
| 5881 | ttttggccaa tttaagttta tgtaggcagt taaatcgatt ttggttaaat cttttgctgc |
| 5941 | taacaaggta tttccagatt ttgaaaagtg gggtggcctg gtgcctgtag taccagcact |
| 6001 | ttgggaggct ggggagggtg gatcacctga ggtcaggagt tcgagactag cctggccgac |
| 6061 | gtggtgaata caaaaattag ccaggcatgg tggcaggtgc ctgtgatccc agctgcttgg |
| 6121 | aagtctgaag catgagaatt gcttgaacct gggaagcgga ggttgcagtg agctgagatc |
| 6181 | acgccactgc actccagctg gggcaacaga gcgagactcc atctcaagaa agaaaagtgg |
| 6241 | ggtgtttagt cttcaaactc cgtgtttaag tgactggagt gaaaatgtaa atcataggcc |
| 6301 | ggtgttggtt taaaaagcat catctgaaaa taatgctgta gtctgcaatt atttttatta |
| 6361 | cgatacgatg gtgtaaaata caagcagatc agtgaaccat tcatgaaaca ttaatcctaa |
| 6421 | aggcgtctca ccccaagtct atcccacaat ctccatgaga cttcgtggaa ccactgtaaa |
| 6481 | gtttcttgtg taatatccca gaagtttcct acctctggta tcttttgaac ttgttgaaaa |
| 6541 | ggcttttcca ccccctcttt atgatggttt gaagagtgtg aacatctgaa tgatgctggg |
| 6601 | gtgaaactgc ttcataacac ttccattttc tcccctattt atttccatat ttttattttt |
| 6661 | tcactaatat ccccacggtt ttacttctgt tttagtaatt cacatgttgc tggactaatt |
| 6721 | ctttttaact gacttgtaac agatatgtta aaccgtttaa aacttggggg gtatttttaa |
| 6781 | cctactttaa gttagttcaa gttaatcagt ctacatggca tataaacctt atgattaata |
| 6841 | aatcttaaat gctggtagct gagttggaag ccaaagacgt acaaaaaagc tgaagtgtta |
| 6901 | ggtttagtgt gataagcttc tcttactaac agggttttgt aatagcagaa atagatatat |
| 6961 | gcatatatat gtgcatatat atagcatacc ttattggatg tccatataaa aatgtgtaag |
| 7021 | aagttaaatt tactgcaaaa tttcttggga gtgcaatttg aagatgatct taagtggtga |
| 7081 | tagtagtttg ctacactggg ggatagttgt tgcaaactgc tcctaatttt cctttactgt |
| 7141 | gaagtaaact gaacagctgt aatagggatt aggaactgta ctccctctct ctctttttta |
| 7201 | agtataatta agtggttttg gggtaagggt gtagggagtg agtgtctttg aagttttgca |
| 7261 | tatactagat gaatgccaca tgtataaggg aggaacaagg gattcttgga aatatttttc |
| 7321 | aatccaagta actttggagg cttccaagtg gagttcattc ccctgtgtag gaaagtgctg |
| 7381 | gggtagaccc ttaaattcct ttctgagcca ttgaaagaat gtcctcaaac ttcgcttata |
| 7441 | ctttatagtt catttagata caaaagttac aaactgaatg ctatttagga aacgtaatac |
| 7501 | actgacatac cgctctttaa atagattata aatttagtat atcaattttc tggcattttg |
| 7561 | ctgaatttta ttgtttagtt ttcaagccca actatcttgt tactttgtat atcgtagttg |
| 7621 | tcccccgttg atcactgttt cctgcttaat tgtgctgtcg tttttcctgg gtcctgattc |
| 7681 | agagtgtcag cattctgttc cccatagaat aagaagaggc tagaaagttt acagatgaga |
| 7741 | tatctaggaa tgccagaaga tcaggggtca ccgttgaggc agagtaatta attatggtta |
| 7801 | aaatggtgtt gctgataagt gggtgctggg aaataattaa aatttgattt tttagaagaa |
| 7861 | tacttctcat gcttgaagag cgccctcatt atatgctaaa gggcctcagg tttttcctta |
| 7921 | ttgccattat gctgcagatt ctattacatt tgtctgaaaa gatctaagac agaagggctg |
| 7981 | tttaatacct tcccttttct cctgaacttc ccctctcctc tcccccatca ggagctaagt |
| 8041 | aggaacccct tcaccttgtt accatcagat ttcatcaatg gtctgtcttt acaatgaagg |
| 8101 | aagtagtact gcattctggg cagaggccag tcctgaggca tgccttttca aggacattgt |
| 8161 | tactttagtt acactggctc ttctgtttta actcttatcc cccagactct aatcctgttg |
| 8221 | ctttttttgg tccccatctc ccacctttca tcatctgaaa tccattcatt gtaacttctg |
| 8281 | gaactcagtc gttagaaaat cctttatatt ctcaatcttg tgaatgttcc tttctttctt |
| 8341 | attccagctg taacctagcc ttctccccaa gaatgctact tcccttgcag ctctctcaag |
| 8401 | tggtgaattt ttcccttctt gcacacctta taacactgaa ctaggaggtg tgtggactaa |
| 8461 | atgtctgctt ttgttcctta ttgtcacttc ttgaccttta ttttccaaaa cttcaagctt |
| 8521 | tgactttcat gtgatcaaat tataccaccc actgcctgtc tttatttcaa gcacctgcaa |
| 8581 | accttcctgg gtcattcaca tccttctttg ttcacttcat tagctcttgg ctcattgtca |
| 8641 | ctgtctctta tttctgtcat aattcttggt gacatcagta tctatgtaga gcaatactag |
| 8701 | tgaagatgtg gtctggtaac tgttacctgt atgaattaag ataaggagtt atgccagaat |
| 8761 | ataagtcacc tgtgtcacta agtttactgt ttagcttact ttttttgtag caagattttg |
| 8821 | atgaaggacg caatatgttg atttacagtc tggtacaaat tttgatgtag aagatgcttc |
| 8881 | caatatcctg gtctcttagt tccttgattt cttctccagt gatcttattt tctaccctaa |
| 8941 | ctcaactaca tattcccatt gtcatatcct agaatatttt gtcttttatc tgtaactctg |
| 9001 | ctctcttccc ccaatctcat ttcaagcatc ccactttcta attcctctag taaatacgtc |
| 9061 | agttccaaca gcccatcaat cccattggga cctacagttt atctatccaa gcttttccct |
| 9121 | gttcctcacc ctcacttcta tacagctgaa gtttcatact gaattataat cactttctcg |
| 9181 | tatacacgtt taacaatctt gtccctccct ggcttcatgc ccagtgatct cttgtatcta |
| 9241 | tgaccatgtc ctttatcttc tcctctgtca ctggatgaac tgtagccttc caagataagg |
| 9301 | ccactcagtt catttgtaca gcagattcca tcccctcttg ctctcaagaa tattactgtg |
| 9361 | gtatctctct tttcttgtct ctactggctc tttccatgag caaacatggt attatcccat |
| 9421 | tacaaaaaaa attttttctc cgtctctcct tccactcacc acctcagtct ctgcttctct |
| 9481 | ttcccgcaaa ataaccttga aaaattgctt tatgtactcc cgttttcttt tgaacccctg |
| 9541 | ccagtgacca ccacgttata aatttgtagt tgtcatctca cttaatctgt tagtagtatt |
| 9601 | tggcaccatt gctacagttg cttgaaatgc cttttcattg gtttccaggc caccatgtct |
| 9661 | gttagcagct tttcctctta cttcactagc atttccttct ttgttttttc tgttatcttt |
| 9721 | ctgacctctg ttggagtggc tgaaggttta gtccttgaat ctttttttgt tgtgcatatt |
| 9781 | tactccagta tcatagcttt atacagatgg tatttacatc tgtttgctaa cgatttccaa |
| 9841 | attggtatcc ttaaactggt atccagctat tttttggtca gcattttgga tgtctaagaa |
| 9901 | gcttctcaaa ctaaactgac ctcccggttt tccccaaagc tgcatcttag tcttttccga |
| 9961 | aatgcaattc tgtctttcca gttacctagc ttaaaagctt gcagttcttg actcatcttt |
| 10021 | ctctcatacc acgtatctga attctctctg caaaaaattg tctgttctcc cttcagaata |
| 10081 | aagtcacgtg tcattttatg atggggatac attcagaaat gcgtcattag gagataatca |
| 10141 | tggttgtgtg aacatcagag tatacataga caaacctaga tggtatagcc tactacacat |
| 10201 | ctaggctata tggtgtggcc aattactatg atgaatactg taggtaattg taacataaag |
| 10261 | gtaggtattt ttatctaaac gtattgaaac atagaaaaag tacagtaaaa aatatggtat |
| 10321 | caaaaataaa aaatggtaca actgtataag gcagttgtga tgaatggagc ttgcaggata |
| 10381 | tgttgctctg ggtgagtcag cgagtgacga ttgagggaac gtgaaagatg tgggacatca |
| 10441 | ctgtacacta ctgtagactt tataaacact gtacacttgg gctacactac atttttgtaa |
| 10501 | ggttttaaaa gacttttttc tataataaac cttaaattac tgtcactttt ttactttatg |
| 10561 | aattcttaat tttttaaacg ttttcactct tgtaataaca cgtagcttaa aacatacatt |
| 10621 | gtacagctgt acaaaaattt tctttatatc tttataagct tttttatatt tttaaaatta |
| 10681 | ctttttacct tttagctttt ttgttgaaaa actaagacat gggccaggcg cggtggctca |
| 10741 | cgcctgtaat cccagcactt tgggaggctg aggcaggcgg atcacgaggt caggagataa |
| 10801 | gagaccatcc tggctaacat ggtgaaaccc cgtctctact aaaaatacaa aaaattagcc |
| 10861 | gggcgtggtg gcgggcacct gtagtccgag ctacttggga ggctgaggca ggagaatggc |
| 10921 | gtgaacccag gaggcggagt ttgcagtgag ccgagatagc gccactgcac tccagtctgg |
| 10981 | gcgacagagc ggaaactccg tctcaaaaaa aaacaaacaa aaaactaaga catgaacaca |
| 11041 | ttagcctagg cctacagagg gtcaggatca tcagtatcac tgtatttcca tctccacatc |
| 11101 | ttgtccttct ggaatgtctt cagaggcagt aaacataaat ggagctgcca cctcctgtga |
| 11161 | taacagtgcc ttctggaata cctcttgaag gacctacctg tggctgtttt atagttaact |
| 11221 | tttttttttt aagaagtaac agaaggagta cactctaatg ataaaaagta tagtaagtac |
| 11281 | ataaacctgt aacaatcatt atcattatca agtgtcatgt actggacata actgtatatg |
| 11341 | ctatactttt tttttttgag atggcatctc actctgtcac ccaggctgga gtgcagtggt |
| 11401 | gcgaggatag ctcactgtaa cctcagactc ctgggctcaa gtgatcctcc tacctcagcc |
| 11461 | tcccaagtag ctgggactac accaggcacc ccaccatgcc tggctaatta aaaaaaattt |
| 11521 | tttgtagaga cagggtctca ctctgttgcc agggctggcc ttgaattcct ggcatcaagt |
| 11581 | aatcctccca ctttggcctc acaaagtgcg aggattacag gtaagagcca ccatgtctgg |
| 11641 | cccactgtac ttttatacaa ctgaagcaca gtaaacctac tgtggtttcg tttacaccag |
| 11701 | catcaccaca aacaccatga gtagaacatt gtgctgcgac gttaacgatg gctacaacat |
| 11761 | cactaggtga taggaatttt tcagctccat tataatctta tgagaccact gttgtatgtg |
| 11821 | cagttcatca tccactgaaa tgtccttatg tgatgcatgt cttcatatcc aaaaatatta |
| 11881 | atcatttctc actgaagcca tgccatgcca tgccatcttt tgcctgtatt attatttttc |
| 11941 | agcttttatt ttagattcag ggtgtacatg tgcaggtttg ttagaaagag tatatcgtat |
| 12001 | gatgctgaag tttgggatac agttgaacca gtcacccagg tagtgagcat agtactcaat |
| 12061 | agataacgtt ctaacattac tcctccttcc ctccctgttc ttgtctctgt ctattgtatc |
| 12121 | tttatgtcca tgtgtaccaa atgtttagct cattcttgtg agaacatgtg gcatttgatt |
| 12181 | ttgtttctgt gttaatttgc ttacaataaa tagtctccag ctgcatccac attgctacaa |
| 12241 | aggacatgat tttgttcttt tttataggct gcatcatatt ccatggtgta taggtaccac |
| 12301 | attttcttga tccagtctac cgttcatggg catttgggtt gattgtatct ttgctattat |
| 12361 | ggatggcttt tgcctatatt attggaaagg ccttctaact ggtgtccctg cttacaccgt |
| 12421 | tttccccctt aaatgtgttt tcaacatggt agccagagta acccttttta taacaataaa |
| 12481 | tcgtgtaact tttttgttca gaaacttaca gggcttacca tttcattcag taaaagctca |
| 12541 | agctcctgta tagtcagacc atatccttca tcacctgtta cttttctcct ctgactcttc |
| 12601 | agcctttttg tttttcctca aactgatgaa gccttcatgg ctgatgtcag atgttttgcc |
| 12661 | cattgagatc ttccttgttg actcagttgc acttggtcat atgattttca tttatttggg |
| 12721 | gtatctaatc ataatctgaa agttggctac ttatttttac ccctttgagg gtccttgccc |
| 12781 | tgtttttgta tccctgatag cgggacagcc agatatctgg aacttacagg tgttcaataa |
| 12841 | agttttgttg aatgaatatt ctggaatcac ccaacctttt ttttcccctc cacttatttt |
| 12901 | tcttctccct ttcacggcct gaaagatgtc ctatgtatat ggttccactt atcactctca |
| 12961 | tcccagtttg tgatatacta ttccattata ttactattat taatacaatt ccattgaact |
| 13021 | tgctcttgct gacttcacca ctggacctac atgttggcca aatggatact ttataatttt |
| 13081 | agtcttgacc cctgcctttg gcacatttct tacctctagc acagcactgt ccagtaatcc |
| 13141 | acactttctg agacagtgga aatgttcagt atctgtgctg ttcagttggt agcaaccagc |
| 13201 | tacccatgcc tattaaacat ttgaaatgtg gctgtgtgac tagtggcaat tatgttggag |
| 13261 | agtacagttt tagaaactcc tgtttttctt acatggcact acatttagta tcacaatcta |
| 13321 | attgtgcaag ccagataggt aggagtcatc tttattcctg ttatttaatt tttctcatct |
| 13381 | actatatcca gttcatcaca tcaacagcgc ctgttgtttc tacctcctaa atatttcttt |
| 13441 | agtctaacta ctacttgtcc ctagtgccac caccatctat cagctggaat attgctatag |
| 13501 | ctgccttaca ggtttccctt ctttcctgtt ctcttctagt tttttgaatt ttagtcagca |
| 13561 | cgagatttta aaaactcaaa taagattgtg ttattcacct gcttaaaacc tttcatgact |
| 13621 | ttcagtgtca cgtagaacag aaaacacttt tcttaccaaa ggctagagag ctctacgtga |
| 13681 | tctggctatt tttaacgttt cattgcactc acccttttcc tctataatca aactactctg |
| 13741 | atctcaaggg ttagttcttg aaagatgatc atgttcttta atgactttag gtttttgtgt |
| 13801 | gttattttct atttctggga tgtttattct ctgttcctta catgctggcc cttttgcatc |
| 13861 | cttcttcagg tctcagctta catgttacct tcaagaagcc tttgaccact ctaagtgggc |
| 13921 | ccttccttcc acttctgctg tgtaatccca ctcccttctc ccacttgtta attagttaca |
| 13981 | tacttttttg taattgttta tttggttgct gtctccctct caagaatgca gggaccatgt |
| 14041 | ctgcattctg cagtaatcac tactgcacac ccagaatcta ttacagatcc tggcatgtag |
| 14101 | ctgatgcata aatatttgtt gaatgaaagt ctgtacattg tatttatgct attggtattg |
| 14161 | ctatgacctg aaactaaaag gagttgtgga aaagatttct tatggaacag aaatatccct |
| 14221 | tttgattaat atcacaatct cgtaaattga gaaaacaaaa aaatatatac tactggagca |
| 14281 | ttcatgtata gttggagatt atgactcatt tattggtgtg tttttggact cagaacaaag |
| 14341 | atgagggaat attccttaaa gctctgtatt gaaataacga aaagcagtca cattttaata |
| 14401 | atagaagctt cctagcttac tctttctgta atcttctttt cctaaatgta agagagcctc |
| 14461 | ataattatga ggcttattac tagagtaagg ctgtcaaagg cagcaaaatg tctttctgtt |
| 14521 | tggaagaata acataaactt gacatgtatg gtgggggaca gaaggtttca aaagtttaag |
| 14581 | aatctgtgtt gtcttaacaa atagatgctt ctcaaggagc ttacgctagt ggttactctg |
| 14641 | tccagtcagg gttttttctt ctttaacttg ggttcatttc ctgatggcac acatgaagtt |
| 14701 | tggatcatat ggtttgactt tagctatggt ccttagctat ggggagcagc atcagcgacc |
| 14761 | tgtgacatgt aaattaaaaa tacaatgcca gggcccttcc ccagcccctc tgatagagaa |
| 14821 | cctcttggcc atctgtattt ttagatgttc caggttagtc tgattaacac ccttggttaa |
| 14881 | gaaccattgg gaggatctga ttgccagttt aaggggacct tcaagcctgt aggtctttat |
| 14941 | agttaaaaaa aaaaaaagat tttaaaaatc atgcatatgt tgtggctgaa ttctggttta |
| 15001 | gcacatactg cttttaatgg cctgaaatgt ttttcccaaa taaattgtct tgttatagct |
| 15061 | ttcatgtgtg atttggtcca gcttcttgtt ttgaagatac ttacgggggg gaacactttg |
| 15121 | tgatttctct tagtaacata ttaacccact taaaaaccct ttctattaca ggtcttcaca |
| 15181 | tttaggctta atgtgcttaa ttcaaatgta aaaatacacc tgcctttgtt ctcagtgaaa |
| 15241 | gtatgtaata aataaatgag gggttggcaa actactgccc accatctgtt tttttatggc |
| 15301 | ctatgaacta agaatcgttt tggatagcta aaaaaaaaaa tcaaaaggat aattattttg |
| 15361 | tgacgtgaaa attatatgaa attcaaattt cagtttctgt gaatgaagtt ttaatggaac |
| 15421 | acagccatcc atgcttatgt aagtgtgcat attctctggc tgttttcact gcaatagcag |
| 15481 | agttgagtag ttgtgacaaa gagtttatgg cccacaaaac ctaaaatatt tactttctga |
| 15541 | tgctttacag aaaaagtttc ctgaacctta ttctagctat atgttgttca taaatgaatc |
| 15601 | tttcgtggtt ctgaaggcat ttaagaatct cttaggttat aaattggctg ggcgcagtgg |
| 15661 | ctcacgcctg taatcccagc actttgggag gccgaggctg gtggatcacg agggcaggag |
| 15721 | ttcaagatca gcctagccaa gatggtgaaa ccctgtctcc attaaaaaaa aaaaaaaaaa |
| 15781 | aaaaaaaata gctggggttg gtggtgggca gtaatcccag ctactcggga ggctgaggca |
| 15841 | gagaattgct taaacccagg aggcggagga tgcagtgagc caagatcgcg ccactgcact |
| 15901 | ccagcctggg caacagagtg aaacaccatc tcaaaaaaaa aaaaaaaaaa aaaacactct |
| 15961 | taggttataa ataattgttg ttagctctcc aagcctccat attacatttt gtgtgttctc |
| 16021 | ctgttcacat tttgagcatt ttatttttta ttagcacatt cagttcatca ggtatttaag |
| 16081 | agcttaatat atgccaaagc atatattaag cgagaagctg tttctaaatg tactgtctca |
| 16141 | gccctcacag agttcacttc attaggctct ttaaaatttc tttctttaaa aggtcagcgt |
| 16201 | gctggtatag tggggaaggg aaactcttac aacacgtcga gtagaggaag gttatcatta |
| 16261 | tgggatataa tttggaagtc attgagtacc tgccattaat tctgcctgta gtctgaatgt |
| 16321 | agagattaac atgtagaaac ttttttgaaa taaaatcttc aatttctttg gcatatctag |
| 16381 | tactgtctag ctaggcatat agtcaaagta tggtgtatat ttcaagtatt aaaagttttt |
| 16441 | ttgggctgta gtcactgttg aaaggatata gttctttact attacatgtg atacctttat |
| 16501 | ataaaattgg ctaacccctg tctttcattt atctgcaaca ctgactgtta ccagttgtct |
| 16561 | ctaactttgg tatggggggt ggaaatatga ttagattgaa agggtacatg actgagccac |
| 16621 | aagcagacct ggatttgaat tttaactgaa cggtttatta gctattctta cattaatact |
| 16681 | gctaatcagt tttcttgtga tatgaggaat gatgtcttct ttatgaggtt gctaggaaga |
| 16741 | ttcaatgaga taacatacta ggctcagaac tgaagttgct aggaatttaa ttatgctacc |
| 16801 | ttgttaaagt atgtcaaagg cagaattcag tgtttagctg ataccacaag gcagtatcct |
| 16861 | aaaattatgc tgtaaaagat ataaagatgc tgtaagtgac tcagaaacct agtgactttg |
| 16921 | taatgcagtt gattcttaga atactgtcac tttaacagaa taggagctag gaatgaagaa |
| 16981 | atagttatta aattactaaa atagaaaatt tattgacaca tgtaaagtga catttgctta |
| 17041 | aatattgaaa aatttgtagt actatttcct tgctttagaa aacattggtt accacttttt |
| 17101 | ttatttatag cagtttgttt ttgccttgag gcaagatggt tgactgagta gttgccacat |
| 17161 | ttcttttgta caaagtccat ttcataggcc atctagcttt tatgcttaga aacatttcct |
| 17221 | taacgttata tttcagtatt tggctaacct atatagggtt aaattatata ggctaacttc |
| 17281 | tcggacagat atttctaata atttatgtat ttggttctgc aaatgtatgc aaaaatatat |
| 17341 | gtacaaaggt atgcagatgc cttgcatact tgatatatgt taaatttttt ttaatgtaga |
| 17401 | cctttttcgt tctctttaat gactatatgg tattccacca tcccccgctc acctggacaa |
| 17461 | ctacagtaac ctcctaaatg gtgtttctac tttgctattg ccccttattg tcttttttcc |
| 17521 | cctttatagc tgctggagtg aattttagaa agcctaagtc atacatcaca ttgcttcatg |
| 17581 | ggcatcccag tacactttgg attttatttt acatccttac tgatctgatt ctcatctctg |
| 17641 | tctcttcatg gttctctgcc ttctagttac actggtgacc tttcaaaacc tttaccacat |
| 17701 | tgagttcatt ccttactttt cactctttct ctgcctggag tgttctgccc catctttacg |
| 17761 | tggccagctg ctcctcctct gatgaaatgt ctcttcctca caggccttcc ctgaccaccc |
| 17821 | actagagtag cacatcttct acctcataaa cttgtttatt agtatttctt actctaaatt |
| 17881 | ttcttttaaa ttgcttaatt ccctaacagt agaatataag cttcactgta tgtatgatct |
| 17941 | tgttgactct cttactcatt gttattgtaa taccagtaac aaagggtgtt taaaatttgt |
| 18001 | tcagtgggtg aatatatgtt ccatttaatg gataaattat tttttattca gtctcctgtt |
| 18061 | gatggacatt tgaataattt ccatcttttt ctctatgaat gcctcacttg gcatgcttct |
| 18121 | gacagtattg ccacagaata catttctgtt ataaaaattg aatttttaag tcaaagggta |
| 18181 | gttacacttt aatggatagt ggcagcttac tatcaaaagt ttctgctagt ttcaccatat |
| 18241 | ccttattagc agtagatatt atcaatcttt tcaatctttg ccaatctgat aagcaaaaag |
| 18301 | taaatgggtt taaacatcct ttgtatatat tcattgctca ctttatgttt ttcctttgaa |
| 18361 | atgttatttc ttgttctttc cctgcagtat gattctttct ttttttgact tgttcccagt |
| 18421 | tttttgtgta ctatggatat tagcctttaa ttatgttacg gatgttctag tatgttattt |
| 18481 | tttgaattac ttcaaatgtg atttgttgct cagattttaa aaactacata cacaaattat |
| 18541 | ctcatgtttc cctttttggt ttcaatttcg actcatgctt aatcagttca tcgattgggc |
| 18601 | atggttttat tcttaatata tacccgtatt ttatctcatt ttattttttt acgtgtaaat |
| 18661 | atttggtgaa tataggttta attttaatgt aaaataagga tgaaaaatga tagttggaat |
| 18721 | tacaagccca tttctcctaa tacttttaat caagtaatcc actaattgaa atattacctt |
| 18781 | cttcatttat gaaattgcca cattatatct gggtgttttt ctgcctacta cagtctctta |
| 18841 | cccatttctt tcctaataat acaatacttg aattgctgtg gttgttgatt tataatgtta |
| 18901 | tcttaatgat aacattataa atgtgatgga actggttcct ccttatagtt cttcttaaat |
| 18961 | caagaacaag acatatcttc ccatttactc tcgtatgtat ctcattttac tgttatgaat |
| 19021 | gaaatctgtc ctatttgtgt ataggaaaat agtttttgta tgtaattgtg atatggccag |
| 19081 | ttttattaaa aatttggtta aactaagagt tgttttctgt tcagccttat catactataa |
| 19141 | aatccacata aaatgggtat aaaagtgtcg caggacactg ggctcagatg attctcccac |
| 19201 | ctcagcttcc caagtagctg ggactacagc ggcatatgcc accacaccca gccaattttt |
| 19261 | aaataagttt taaaaatagt atttttagta gagacagggt ttcaccatgt tgcccaggct |
| 19321 | ggtcttgaac tcctggactc agacaatcca cctgccttgg cttcccaaag tgttgggatt |
| 19381 | acaggtgtga gccaccacac cttgccgaat tgcagccata tttaatactt ttttccatcc |
| 19441 | tattcccttt gctgccccca ggcctcctgt attgatagcc cgctattaag aagctagtgt |
| 19501 | atattctttg catactttta cttcataaac tatatgaagc attgttctgt tttttaactt |
| 19561 | aattggtata aaattatatt ttggaaattc agtatattct gtgaaaatta tttagaaaat |
| 19621 | gtgcctctga gataaagcct attcaggatg tatcttaaag gagatagctg tgctttaaca |
| 19681 | ttatcagtct ttttggctgc ttatgttaat ataagttgga gaaaaacagt ctgctttttg |
| 19741 | tgataatatg ttcttggaga tggagtgaaa gattgtttaa aaacattgtc ttttttttcc |
| 19801 | cctgaagtac cagtatttat tttaggatta tgttactgat caaagatgct gtgtggagtt |
| 19861 | actcattggt gagactaaca ataaatcaca catgcaaagg atgttaccat aatctaatta |
| 19921 | ttttaaacag taaaattata ttctaagaca tccagttggc ctatatgtgc tatatcaatg |
| 19981 | actatcaagg ggctttttat gtatactgta tacatgtact tcacaaaaat ataaaaggat |
| 20041 | gacatcaaaa atctggcaag ccaaaagcct acattacatg tagcaaataa ataagcatat |
| 20101 | gaacttattg gaatttaaaa ccctgtagga tgggcgggtg atggtatgta tgttagatgt |
| 20161 | gtggacatat ctattaaaag ttgtgtcaga taacagctgg tgctgacaag cccttggtaa |
| 20221 | gatggcagca tgttcaatat gttctgtgaa aattatctca gtttatgatc tgtcagtatt |
| 20281 | gtggagctat gcatgaaagg acttaaaatt cttaccctta aactcagtaa cagtgtttct |
| 20341 | agaacttctg gtgatatggg aaattaagag aattatttat atgcaaaggt gtttattgca |
| 20401 | gcattgttgg aataatagac aaaatgggga agaacaagct cagaatggag gaggtagctt |
| 20461 | atagtataga catacgatac aatccagatg ataatatttt ataatagtct tcacaaggaa |
| 20521 | ttttatattt ttatttttaa aaatacatag cagtgagttt aatataccaa acataccaaa |
| 20581 | atgtcatcat ttactgtgtg gtggactcat atgatggaga tgataaataa aaatattaat |
| 20641 | ttatttgagg catatattta tggctgagga aggaagacag ttatgaagaa cagctcattc |
| 20701 | tggaaacata ctaatttttc ccagccataa agagatttcc tatttctttt ttttttccat |
| 20761 | ttaccttctg tttcctacct gagaagattt catacttcta ataaccattt gtgtacctat |
| 20821 | ttaaagacag taccaaaggc atacatttta gtgtttggag gaccaagggt catttgatgt |
| 20881 | ttgatgctta ttgactattc gaggatgaca agacaccttg agaacacaca cacccacacc |
| 20941 | cacacccaca ccctcaccca cccaccccac ccccctcccc gaagaaagct gtgaaggaag |
| 21001 | aaagcagaaa agaacctgga gtgagttgta acttaaaatg ttagtgttgc atgaagtgtg |
| 21061 | ttaaaacagg aagatttgag gaaattgcat acattttcta gatggcaaag tattactggt |
| 21121 | gacagttaat gaaaatgcat atgcatgtgt ttttagattt acaaatttta ctaagaactt |
| 21181 | tttaaaaatc cctgaaggtg tatcaaaagt ttatcatgct tatgaaatag agtagcactt |
| 21241 | tctaacttta aaacggggaa taattctttg gatcttgatt attggaaaag tgaattatga |
| 21301 | attgctagta taaaactgtg gttttaaaat atgtctgctt tatattttta tgtagcagat |
| 21361 | ttactcctag ttaataatac tcaaacttac tgaaaactaa ggtaattaag ataattctgt |
| 21421 | cctgatggga agaggaaaaa taacttcagt gtgaaatcta ttatatatta gttgtggcaa |
| 21481 | gatttctccc attgactttg actggagaca tttatagggt taaaatcgga aatagcacgg |
| 21541 | tgaattttga agtatccttg tagttggaaa gagtattatg ttcatattgc caaaaaaaag |
| 21601 | atgcatggat gcattagact ggatggaaaa tacatgagaa gttggctagc cccctctttg |
| 21661 | tcaaaacatc acttggtggt gataaagctg ttggaaaaca cagcattcta atgtagtctg |
| 21721 | tagtttaatg ataatctgtg tcttgaaaca tttagcgtag tacttataca aacctagatg |
| 21781 | gcatagtgta ctgcatgcct agcctatata gtatagcctg ttgcttctag ggtgtaaagc |
| 21841 | tgtatagcgt gttactatag gcagttgaaa cagtggtatt tatgtatcct tttttttttt |
| 21901 | tttaaattct tttaagagac agggtcttgc tctgttgccc aggctggatg cattggtgtg |
| 21961 | atcatagctc actataacct tgaactccta agtgatcctc tttgcctcag cctccccagt |
| 22021 | ggctaggact acaggcacat actaccacac ctggctaatt tttaacattt ttttgtagag |
| 22081 | atggaatttc gctgtgttgt ccaggctggt cttggaactc ttgtgctgca gcaatccacc |
| 22141 | cgcctcccaa agtgttagaa ttacaagcca cttcgcctgg cttgtttacc taaacataga |
| 22201 | aaagatccag taaaaataca gaattaaaat cttgtggggc cactgtagca tatgtagtcc |
| 22261 | atcttgactg aaatgtcctt atgcagtgca tgattgtact tcataatttt taagcactcc |
| 22321 | tccctcttga ttggtactta gtggatttta tcatttttgt ttcttcataa ttctttctga |
| 22381 | aatgtctact ggttggacct ttgatctcct gaattgatcg tgatttcttc tgttgtattt |
| 22441 | tttgtctttg tcattttttt gtactctagg cagttttctc aattttagtt tctattcaac |
| 22501 | tttttgtttt tatttattct ctccagtatt tatggagata ctaaattgaa gtgttctgtt |
| 22561 | tctctctcca ccctatccct agtttcaagt tttatctcag tttctatgga gtcagttttt |
| 22621 | tcgttgcttt aaaaaaaaat tttcctgaag tgattggtaa gttttggcta attgggagca |
| 22681 | ctagaattgg gcccttaatg gttggcaggg tgtggtggag gagagacagc ccttagtcca |
| 22741 | aaggctcagg ccagaaaaag aaagaggaag gctttccttt tcctttccgg agcagggttc |
| 22801 | tgccctaggt cttgcttggc agtctatttg atttctttag cagttaatgc tcagtttttt |
| 22861 | ggcatatgtg gatctgcctc cagagcaggt acaaggtgag tgagtctatg ctgttaccta |
| 22921 | attagatccc catttctacc ctttgttttt acttctctat ctactgatag gtttttaccc |
| 22981 | tccttcacct catagggttg cagtgaagag caagatgaat ttttatttat gttgcataaa |
| 23041 | ttttaaaagc taaaaaatat atatgtaatg ttgggaagtc ccagtgtaca aatggctatt |
| 23101 | gtaaatttgg aacatgaact tgcttttttc cattgtaaaa atgaaatcat tataaattgc |
| 23161 | ggtcaagtta ctaggtcagc ccacacagag tttacccagt aatatgcgta aatgttttgc |
| 23221 | ctttgcatca acaacaagga aaaacagtac tataaaaaaa tgttcctgga agccggatgt |
| 23281 | atcaaagcac ttctgaaata gctatatagc ctatagacat gaccagttgg tttctgagtc |
| 23341 | tgttgacatt ggccaaagga gaagctcagt gtagaacatg tttggagtct ccttttgcag |
| 23401 | aaatacattg gaggctggag tggggaacca atttttcaga aaggtggtga agtagttaca |
| 23461 | tagccactct tttaaagaca gtcaaaagat agaaactaag gccaggtgtt ggctcacatc |
| 23521 | tgagatagga aaatcacttg aacctgggag gcggaggttg cagtgagccc agtatgcacc |
| 23581 | tctgcactcc agcctggttt ggcaagagac caaaactctg tctcaaaaaa aaacaaaaca |
| 23641 | tagttcacac ttaaatattt tattccatat ctttacatac ccaatatgtt aatttatagt |
| 23701 | tcaagatgaa cttgtttggg acagattttg taataaagga aatcgtgtta ttagaaatat |
| 23761 | ctagaggcca tgagccctta aactgttcta atttgcaagt agttccctgt gtgatgcagt |
| 23821 | ttttttcaat attgcacaat aaaggcaaaa tacggacaaa ttagatgata agatttatat |
| 23881 | aaatttttaa aatattgatc aaaatatgta tccatattgg taatatttgt atttataata |
| 23941 | aatcattgct gtaaatttga acttagaaaa attttactaa taaaggtgct tttgtgttgc |
| 24001 | aaactttcat ttgaaaagta atttttcttt gtaccaaaaa atctaaaatt cgctattcta |
| 24061 | gtcaccaaaa tttgctttat gaaaaataat ttttgatggc actatatcag aaaacaactt |
| 24121 | gttaaagaaa atgtggagtt tttaaaatcc cactgtacct ctgttatcca aaggggatct |
| 24181 | gtgaattttt ctgtgaaagg ttaaaaaagg agagaccttt aggaattcag agagcagctg |
| 24241 | atttttgaat agtgttttcc cctccctggc ttttattatt acaactctgt gctttttcat |
| 24301 | caccatcctg aatatctata attaatattt atactattaa taaaaagaca tttttggtaa |
| 24361 | ggaggagttt tcactgaagt tcagcagtga tggagctgtg gttgaggtgt ctggaggaga |
| 24421 | ccatgaggtc tgcgtttcac taacctggta aaagaggata tgggtttttt ttgtgggtgt |
| 24481 | aatagtgaca tttaacaggt atcccagtga cttaggagta ttaatcaagc taaatttaaa |
| 24541 | tcctaatgac ttttgattaa ctttttttag ggtatttgaa gtataccata caactgtttt |
| 24601 | gaaaatccag cgtggacaat ggctactcaa ggtttgtgtc attaaatctt tagttactga |
| 24661 | attggggctc tgcttcgttg ccattaagcc agtctggctg agatccccct gctttcctct |
| 24721 | ctccctgctt acttgtcagg ctaccttttg ctccattttc tgctcactcc tcctaatggc |
| 24781 | ttggtgaaat agcaaacaag ccaccagcag gaatctagtc tggatgactg cttctggagc |
| 24841 | ctggatgcag taccattctt ccactgattc agtgagtaac tgttaggtgg ttccctaagg |
| 24901 | gattaggtat ttcatcactg agctaaccct ggctatcatt ctgcttttct tggctgtctt |
| 24961 | tcagatttga ctttatttct aaaaatattt caatgggtca tatcacagat tctttttttt |
| 25021 | taaattaaag taacatttcc aatctactaa tgctaatact gtttcgtatt tatagctgat |
| 25081 | ttgatggagt tggacatggc catggaacca gacagaaaag cggctgttag tcactggcag |
| 25141 | caacagtctt acctggactc tggaatccat tctggtgcca ctaccacagc tccttctctg |
| 25201 | agtggtaaag gcaatcctga ggaagaggat gtggatacct cccaagtcct gtatgagtgg |
| 25261 | gaacagggat tttctcagtc cttcactcaa gaacaagtag ctggtaagag tattattttt |
| 25321 | cattgcctta ctgaaagtca gaatgcagtt ttgagaacta aaaagttagt gtataatagt |
| 25381 | ttaaataaaa tgttgtggtg aagaaaagag agtaatagca atgtcacttt taccatttag |
| 25441 | gatagcaaat acttaggtaa atgctgaact gtggatagtg agtgttgaat taaccttttc |
| 25501 | cagatattga tggacagtat gcaatgactc gagctcagag ggtacgagct gctatgttcc |
| 25561 | ctgagacatt agatgagggc atgcagatcc catctacaca gtttgatgct gctcatccca |
| 25621 | ctaatgtcca gcgtttggct gaaccatcac agatgctgaa acatgcagtt gtaaacttga |
| 25681 | ttaactatca agatgatgca gaacttgcca cacgtgcaat ccctgaactg acaaaactgc |
| 25741 | taaatgacga ggaccaggta agcaatgaca tagctagctt tttagtctgc tttgaagtaa |
| 25801 | atgctcaagg ggagtagttt cagaatgtct acccaatacc agtacttgaa aactaacgat |
| 25861 | gtttctgaat tcctgtatta caggtggtgg ttaataaggc tgcagttatg gtccatcagc |
| 25921 | tttctaaaaa ggaagcttcc agacacgcta tcatgcgttc tcctcagatg gtgtctgcta |
| 25981 | ttgtacgtac catgcagaat acaaatgatg tagaaacagc tcgttgtacc gctgggacct |
| 26041 | tgcataacct ttcccatcat cgtgagggct tactggccat ctttaagtct ggaggcattc |
| 26101 | ctgccctggt gaaaatgctt gggtaagaaa acatgtcaga atgcttgaag ctaaaaagta |
| 26161 | gaagagtata ctcacaatat ttctgatgag gcttttttct tcttcccagt tcaccagtgg |
| 26221 | attctgtgtt gttttatgcc attacaactc tccacaacct tttattacat caagaaggag |
| 26281 | ctaaaatggc agtgcgttta gctggtgggc tgcagaaaat ggttgccttg ctcaacaaaa |
| 26341 | caaatgttaa attcttggct attacgacag actgccttca aattttagct tatggcaacc |
| 26401 | aagaaagcaa ggtaagagaa ttattcttta tgtggttttc atggagcatt ggacacctcc |
| 26461 | agtgtcatgt cattccatgc agtgttccta acctttttgg caccagggac cagtttcgtg |
| 26521 | gaaaacagtt tttccatgaa tgggttgtgg gaatggtttc tggatgacac cattccacct |
| 26581 | cagataatca ggcattagat tctcataggg agcgtgcagc ctagatccct cgcatgtgca |
| 26641 | gtccacacta gggtttctac tcctatgaga ctctcatggt gcagttgatc tgacaggagg |
| 26701 | tagagctcaa gccaggtaat gctcgctcac ctgccactta cctcctgctg tgcagcccag |
| 26761 | ttcatttctg ttcttttaaa tttttgagtt tccatatgta aagcactatg cgaagtagta |
| 26821 | gggatatggt aggcaagctt ctcttcacac ttttgttctt aggtgggatg tagatgttgg |
| 26881 | gaataataac ctaatattta atttgtgtag tgggaagaag tggggctatg agggcacata |
| 26941 | acacaagttg aaactgactc tttttgaggg ttcaaggaga cctcttggag gaagtgatag |
| 27001 | ttgagttcag tgttcaagga tgagaaggga ttcactaggt gaaggttagg tgagaaaaca |
| 27061 | acatctttga aacgaaggaa ggagatggaa agttttggga atttaagaaa tactaatagt |
| 27121 | aaggaggaag aaaggtttga ggtgaggcta ttgagataga cttagcagat ctcatagggc |
| 27181 | tttgtagagc atgtttaaaa gcacaatggg aaatttcagc agaagcctga aatgatgaaa |
| 27241 | tttgttttta gaaaattggg gcagtgttga aagggaagat atacagggaa tgaaaggaca |
| 27301 | agcatgaatg atcattttat ggtatctgtt tttaaggtgg atataattag gaaaattaaa |
| 27361 | gggccaaatg atgaggagtt aagtgccagt tctggttcaa attttcagtg aatcagtttt |
| 27421 | gatataactt tcatcttagg gcattactct tgcctaccaa catagtttct aaattttttt |
| 27481 | cttttggtgt gatcactgtg ggaagaagga aattgggccc aaactgatac attgtttgga |
| 27541 | ggactgggat gtctgaattt gagtggaatg ctttaaaagg acaagttgga tagggcccca |
| 27601 | gtatgggggt ctgagtgatg gggtccagga atacatttag gtccaatggc aagctggctg |
| 27661 | aaattcttgt ataataaaat aggttggtaa tatggctctt ctcagacatg tgatcaagat |
| 27721 | tccttgacta acaagatata tatatatatc tttctagctc atcatactgg ctagtggtgg |
| 27781 | accccaagct ttagtaaata taatgaggac ctatacttac gaaaaactac tgtggaccac |
| 27841 | aagcagagtg ctgaaggtgc tatctgtctg ctctagtaat aagccggcta ttgtagaagc |
| 27901 | tggtaagtat atgtatctat tctgagtctt gtgtatagca tctgcagttc taattagatt |
| 27961 | acttttctta ggaaaaggtg gtagaacttt aactactgaa aataaatggt cctattcagt |
| 28021 | ttgcagccaa gatttacatt cagagtacct gtcatctgga ttgtagctaa atatttaagg |
| 28081 | ctagtttagg tagagttctt attatccatc aaaaatgatg gcatatgttt tgcttaataa |
| 28141 | aatttgtttg taatttcagt tttgagtaaa cctaagattt gctaacagag ctgtgaattt |
| 28201 | ataggagaaa agacaaattc taatatagta cagttttatg taaagtgatt gctttattag |
| 28261 | tagatgctca tgagcagttt ttgttttgtt ttaactttta ggttccgggt aatgtgcagg |
| 28321 | cttgttatat aggtaaattg catgtcacag gggtttcgtg tgcagattat tttgtcaccc |
| 28381 | aggcagtaag tattgtaccc aataggtagt ttttcagttc tttacctccc acccgtaagt |
| 28441 | aggccccagt gtctgttgtt cccttctttg tgcccgtgtg tactcagtgt ttacctccca |
| 28501 | cttataagtg agaacatgtg gtatttggtt ttctattcct atgttagttt gcttaggata |
| 28561 | atggcctcca gctccatcca tgttgctgag gaagacatct tggtattttt ttatggctgc |
| 28621 | ttagtattcc atagtatata tgtaccacat tttctttatc tagtctacca ttgatgggca |
| 28681 | tttaggttaa ttccatatct ttgctattgt gaataatgct gcagtgaaca tatgcatgca |
| 28741 | tgtgtcttta tggtaaaaag atttcttttt ctttgggcat atacctaata ataggattgc |
| 28801 | tggattgaat ggtaattctg tcaggttttt tgagaaatca ccaaattgct ttccacaatg |
| 28861 | gctgaactaa tttactttcc caccagcagt gtataagcat tctcttttct cagcaacctc |
| 28921 | accagcatct gtcatttttt gactttttat tagtagccat tctaactggt gtgagacggt |
| 28981 | atctcattgt ggttttgatt tgcatttctc taatgatcag tgatgtcgag cttttcttca |
| 29041 | tatgtttctt ggccacttgt atgtcttctt ttgaaaagtg tctgttcatg tcctttgccc |
| 29101 | actttttaat ggggttgttc ttttttgctt gttaatttaa gtttattgta aactctggat |
| 29161 | attagacctt tgtcagatgc atagtttgcc agtactttct cccatgccag tactttctcc |
| 29221 | cattctgtag gttgtctgtt tactctgttg atttcttttg ctgcgcagaa gctctttata |
| 29281 | ctgtcccatt tgtcagtttt tgtttttgtt gcaacttctc ttggcatctt cgtcatgaaa |
| 29341 | tctttgccag gtcttatgtc cagaatggta tttcctaggt tatcttgcag agtttttaca |
| 29401 | gttttaagtt ttatatttaa gtctttaatc cattctgagt tgatttttgt acatcatgta |
| 29461 | aggatggggt gcagtttcaa tcttggatgt ggctagccag ttatcccagc accatttatt |
| 29521 | gaatagggag tcctttcccc attgcttgtt tttgtttact tgttaggtgt gcggcctaac |
| 29581 | ttctgggctt tcttttctgt tccattggtc tctgtgtctg tttgtatacc agtaccatgc |
| 29641 | tgtgattgta accttgtatt aacagtatag cttgaagttg ggtaaagtga ttcctccagt |
| 29701 | tttgttcttt ttgcttagga ttgccttggc tattcaggct cttttttggg ttcatatgaa |
| 29761 | tttttaaata gttttttttt aattatgtga agaatgccat tggtagtttg gtaggaatag |
| 29821 | cattgaatct gtgaattgct ttgggctgta tggccatttt aacaatattg attcttcctg |
| 29881 | ccatgaaata gaatgttttt tcatttgttg gtgtcatctc tgatttcttt gagcagtgtt |
| 29941 | ttttgtaatt ctcattgtag agatctttca cctccctggt tagttgtatt cctaggtatt |
| 30001 | ttattctttt tgtggctttg gtaaatggga ttgcattctt gatttggctt gcagcttgga |
| 30061 | tgttgttggt gtctagaaat gcttctgact tttgtacatt gatttttata tcctgaaact |
| 30121 | ttgctgaagt ttattggatc aaggagcttt tgggcagaga ttatggggtt ttctaggtat |
| 30181 | agaatcatat tgtttgcaaa cagacttcct atttggatgc attttctttc tcttgcctga |
| 30241 | ttatgagcag tgttttgccc tgatattctg tattctcagt gaatagatgt cgtctaagta |
| 30301 | tgagaaacaa tttttttcta ttctgagtat ttttaagaag gcaacttata tgtggtactt |
| 30361 | tgtatattgt gtatgttggc aattggggaa aagaatagat ggtttgtact agggcctctt |
| 30421 | gggttctgtg tgtgtgtgtg tgtgtgtgtg tgtgtgtcat gaaaacagtt actttttagc |
| 30481 | taccaagcat tttttctcct ttcagtaacc cacctaacaa catttactca gaatttcaaa |
| 30541 | gcaagcttca aatcagtatt gaaagaagga aaaatataaa ggcatttaat ggaagaaaat |
| 30601 | gttgggaata aagtataggg ctggcaacac ttacttttct cacttattga gagtaatttt |
| 30661 | acttgggaat ttatgagaga gaaagacatt atgattgctc caggtaacta ctggcagagg |
| 30721 | aaccatagtc ttggggatag acaaatgtgg ctgagttcat atagaatgag gggatgggat |
| 30781 | gtaaattctg tcagctgttc cagcagtaac ctgtaatgta ggctaaaaat acagattttg |
| 30841 | agatttattt aatcagaatc cctggagtgt taatttttat atcaagatct catagtgttt |
| 30901 | tatttgaagt gacagggagg tctgtagata gctggacatg tatgggactg gaagcttagg |
| 30961 | aatctttaag ttcttccagg ttattcttat gttcatttgt ttattctgaa aatagcatct |
| 31021 | aatgtatttt aagaaatgga ataggcacat agtatacatt gggtaacaca acagataggg |
| 31081 | tccccgtgct taattcttag tcttgtgaag gtgacaaaaa tacttaaaaa tatgtgatcc |
| 31141 | taaattagaa tgagtgttat gggagaaatg acagcaaata gtgatgagaa ttaatgggga |
| 31201 | ggggaattgt ctagatgaga gggaaaaggt ctccttgaaa aggggatgtt aagtgggact |
| 31261 | gcaggatgag agggaaccgt ctcttgtcta tatgagaagt gagggttaaa cgttttccag |
| 31321 | gtagagaaaa ggaacaccat gtgctatgtc ttagaaccag ggatatccag tcttttggct |
| 31381 | tccctgggcc acattggaag aagaataatt gtcttgggct acacaccaaa tacactaatg |
| 31441 | atagctgatg agctaaaaca aaaaaaaatt gcaaaagaat ctcataatgt ttaagaaagt |
| 31501 | ttacgaattt gtgttgggct acattcagag ctgtcctagg ccatgtggcc catgggctgc |
| 31561 | aggttggaca agcttgcctt agaaggaaag agattggtca ggcacggtgg ctcacgcctg |
| 31621 | taattccagc actttgggag gctgaggtgg gcggatcatg aggtcaggag atcgagacca |
| 31681 | gcctggctaa cacagtgaaa ccccatctct actaaaaata caaaaagtta gccgggcgtg |
| 31741 | gtggcaggcg cctgtagtcc cagctacttg ggaggctgag gcaggagaat ggtgtgaacc |
| 31801 | cgggaggcgg agcttgcagt gagctgagat agcgccactg cacttcagcc tgggcgacag |
| 31861 | agtgagactc tatctcaaaa aaaaaaaaaa gggaaagaga ttgtggagat ccaggtgctg |
| 31921 | aagagaaggt ctgcataaac agaacttagt aatgaggtgg atggcctggt atgaggttga |
| 31981 | ggttaggtaa gcagagccat aacatgcagg actttctagg ttcctataag atagttacta |
| 32041 | ctcatggagt ttattcatgc tttattccag ctttggagcc atagatacag aatactttgg |
| 32101 | tcagtttgga aggctaggtg ggatccaaat tctaaacggt tcctcagggt tatactaaag |
| 32161 | tatttctatt atcttaaaag gatgctgaga cactttcgat ggttgtttat caatagcaaa |
| 32221 | gcatcacagt ggtgtgttta aaatattaat aatagcattg tatagattaa cagtttgaat |
| 32281 | gaccaaaagc tagaagacca gactactgag atgttacagg cttttaggaa tgaaatagtt |
| 32341 | tgcttttaga actcaatagc aaagggcaga tgtctgagat gcctgaaaga atcatagaat |
| 32401 | gtaataatat aggagctaag ggagcaacca aaaacggttt gtggagggga caacattggt |
| 32461 | accatgaaga taaatggaac cctcagaagg catccttaat ttttgaacat aataatttaa |
| 32521 | gaagctgact taaagtgact taaaaggtca gtaggtagct ggaaatgtat gatactagaa |
| 32581 | tgcaagagag gcaggctaga gatttggaag tttccctctt agtatatagg ggtaagggca |
| 32641 | gcagggaagg ggaggtagag gtgccacaga gtcatctgta tgggactttt ttttttaccc |
| 32701 | tagaactgct gaatcagaat gtgtgtgttt taaagtctct gtaggccatt ctgatggaca |
| 32761 | tctggggtta aaatccattc tcttagagtt aatagttatg taaagggagg gaatgaagtc |
| 32821 | ttaaagaggg gaaagaaggt agtcatttca caaatactga gcatcctgat catcagtctt |
| 32881 | acgcagatca ttctattagt agctggagct actatgaaaa aggaacccaa cagaggtgat |
| 32941 | ctttgtcttg tagggaaagt ggagtaactt acactatgaa ggagaagtgc agggtaccat |
| 33001 | aagaattaca gcagatagac ctcatctgag gaaataaaac agacccgaaa gatgaaggag |
| 33061 | acaaggaaaa gtatctctta ctgcattcag aagtgattta agttgaagat ggatgagcga |
| 33121 | agttaatcta ctatgtgggc attgggcttc catttatact cctttgccag agtaaatgtc |
| 33181 | ccccatttaa gggtcctaaa ggatggaaga ttgtaaacct tggaacacat gttttgtagt |
| 33241 | cagtgaattg tataaagtcc ctgacagtaa gtgttttcat gccgtctttc tggattgttc |
| 33301 | ttaccccagg aatttaccta gcttctttag gtctttagtc agatgtcacc ttcacagtga |
| 33361 | ggtgacctaa ttatctattt aaaatcgcag ccccactcca ttatttttct ccatagccct |
| 33421 | ttaatatcat ctgacatact gtatggtttt agtttattgt atatttttct gcctcttcca |
| 33481 | actagatcat aaattctgag ggtaggaact tctgaatatt tttgttcact ggtctatctg |
| 33541 | cagctcagaa caggacctgg tactgaataa atatttttga aatgattgaa tggatgaaaa |
| 33601 | gaaatgagta ataagaatat tacctaaggg ggacagtgga gataacaaag gctttttcgg |
| 33661 | cttaggaaag gaacagtagc tatttgagag tttgtcacta gtgaggtgaa ctggcaaagt |
| 33721 | gaaggaaact gagcaacatt ctagaaaatg agaggaaatc aaatacttag gtgaaaggaa |
| 33781 | gtaaactctg gaaatacaga aggacacctc ctaaggctag aacagatatt taggattgat |
| 33841 | aggcacttct agctaatgac tagggcctta tatccttttt aattttctag gtggaatgca |
| 33901 | agctttagga cttcacctga cagatccaag tcaacgtctt gttcagaact gtctttggac |
| 33961 | tctcaggaat ctttcagatg ctgcaactaa acaggtaaat tctgagtaaa ctggtgccat |
| 34021 | gggaatagag tcaagatgag tatgtgcttg tactgaccat ctgtttttat ctccatagga |
| 34081 | agggatggaa ggtctccttg ggactcttgt tcagcttctg ggttcagatg atataaatgt |
| 34141 | ggtcacctgt gcagctggaa ttctttctaa cctcacttgc aataattata agaacaagat |
| 34201 | gatggtctgc caagtgggtg gtatagaggc tcttgtgcgt actgtccttc gggctggtga |
| 34261 | cagggaagac atcactgagc ctgccatctg tgctcttcgt catctgacca gccgacacca |
| 34321 | agaagcagag atggcccaga atgcagttcg ccttcactat ggactaccag ttgtggttaa |
| 34381 | gctcttacac ccaccatccc actggcctct gataaaggta aattgtcaaa gtagaattta |
| 34441 | cctttgttgc agaattgaaa atgaagcatc tctagctgtt ggatggctgt ctaagcatag |
| 34501 | tgatcaataa gtaggaattg tattccttag taagtaggaa gtatggctgc gataggggta |
| 34561 | agattctgaa atgtttgtgt agtcagaact acttttagtt gataccaata gatttagtgt |
| 34621 | ggtgggaatt ttagggtaag aaaatgattt tgttgagttg tatgccagtt cttccttctg |
| 34681 | tttttcaggc tactgttgga ttgattcgaa atcttgccct ttgtcccgca aatcatgcac |
| 34741 | ctttgcgtga gcagggtgcc attccacgac tagttcagtt gcttgttcgt gcacatcagg |
| 34801 | atacccagcg ccgtacgtcc atgggtggga cacagcagca atttgtggta ggtaaattct |
| 34861 | tacagtgata cctggctatc taaaaggaat gcataaatcc aaaggatcct gaacttcttt |
| 34921 | ctttggtcat tggttccccc catccgtctt cctgaagagc taatgacaaa gtaaataaat |
| 34981 | aaataattac acatttctat ggctgcagag aaaataaggc atagtgtggc cccagtgata |
| 35041 | tttccttgga cacgtccttc acatggtcag tcttacaaag gttgggttag gtgtttcata |
| 35101 | aagtgttctc atttaattta cacaaaggcc cacttcctta ggaagaggta gagtcataat |
| 35161 | ttgagatcaa atctgtgtaa tttcagagcc tcttaccctt gcctcatcat gcattttgac |
| 35221 | tataaatatt tagcagtccg ttttattatc ttttctgtga gttaaacttt tttcatggac |
| 35281 | ctaagaatat tcagaaataa gtagtagcat ttctgtactc ttaaccacaa aaatctcaac |
| 35341 | ctgaagcttt gatacaaagt ttgtgtctta aaagtagctt cattaaaagt atagtctaat |
| 35401 | gacatttctg atttctcaga ctttaagacc ttattaggtt agtttagaaa acaaagatgg |
| 35461 | agcctaccag aacagatgtt aggaatctca ttttgctggt tgctttgtgt atgtactcat |
| 35521 | attggggctt tggctttctt catttattac tgttggtatt ggcccatctc catgaggtga |
| 35581 | cttaatagaa cgttgagggc accttttatt ttaaatctct tttctaggaa gaagagagtt |
| 35641 | tttgtgtcct tgtaagaatc aagttattta taaaagctgc taaatgtagc agaataataa |
| 35701 | ccccttttaa aactcaaatc cagaaacagg agaaacagat ggtacttaca tattgcaaaa |
| 35761 | gctatcttcc ttctatacat gaggctgtca gctgaatagt cttggaagag tgaggagtga |
| 35821 | atttttctgc tggcaactcg gttagtttta gcagttggtg ctaaaacttg gcaaagtttt |
| 35881 | caccaaatac atggaagata tacaaaaata gagggggcat gtaaaagaaa aacgttgaca |
| 35941 | tagtctgagc attactttct catcttctct ttttatatac cttttaccca gaatgattgg |
| 36001 | tgcccttact gtaggaaagt tgtctttggg attcagcgct gtatggaagc tctgttgcac |
| 36061 | tgtgtatggg ggaggggtgc tgctttgaat tagtgctgcc aggaggcctc ttttcagtga |
| 36121 | cattcaagtt aatggaatcc ttcttccttc ctgaactaat tgcaagttac ggggaacttc |
| 36181 | gggtatataa tgtaaataat tacagtctaa taattgttcc tcaaacttta cagaggagaa |
| 36241 | tgccctgttt gttaaccatg tttcttttgg caggaggggg tccgcatgga agaaatagtt |
| 36301 | gaaggttgta ccggagccct tcacatccta gctcgggatg ttcacaaccg aattgttatc |
| 36361 | agaggactaa ataccattcc attgtttgtg caggtatgtt ttaagtgaag tgttctaggt |
| 36421 | tttatgtcca taaaatttcc agattgtaat gactaataac atttcagaaa attagggacc |
| 36481 | ataatagggt taccaacatt taattttatg aaaattccct acattttttg gtcagtaaga |
| 36541 | gaaacattga gacttgagaa gagggaggag atttcacatt tcacttttat gggtgcctag |
| 36601 | aggggagagc tgacctgggc tgccagaggc agggcataga cccccaacca attctgggtt |
| 36661 | ttccaaatct tagatcagtt agagctgcct ctgaagaaag ggtttatagc taaaaaatat |
| 36721 | tatggaaatc cagtgctcca gagcattaaa caccccaaga cataaaattc agagaatatt |
| 36781 | atttactaca gtgtgaatgc ctcttgcact ctgaattggg aatgtttgca ccacagtggg |
| 36841 | gggcttgcca tgttttagct ttagatttaa ttaggttttg tttgtgtttt ctccttagct |
| 36901 | gctttattct cccattgaaa acatccaaag agtagctgca ggggtcctct gtgaacttgc |
| 36961 | tcaggacaag gaagctgcag aagctattga agctgaggga gccacagctc ctctgacaga |
| 37021 | gttacttcac tctaggaatg aaggtgtggg taagtaaaaa ggaaccaaag cctttagcag |
| 37081 | atgtgtacat tgaagtctca gtttttcctc aagggccttt ttctccttgt ctcttagcga |
| 37141 | catatgcagc tgctgttttg ttccgaatgt ctgaggacaa gccacaagat tacaagaaac |
| 37201 | ggctttcagt tgagctgacc agctctctct tcagaacaga gccaatggct tggaatgagg |
| 37261 | tagggaaatg tgagcagtta tttatctggt agtttcctag agcaggtatg gcagcttgtt |
| 37321 | ctttcctctc aaaacactta gtacacattc atttgcattg atgtttccct ggcttgagta |
| 37381 | tttcttcttt atgctgtcta gcaactgctc tgaggaagaa ctataataca agctttaaag |
| 37441 | agtctgttca gaatcattac aaataagttg tgttatttaa aattataatt cataagggag |
| 37501 | aaagatgaaa aatgttacca gattaaagaa gatttttcaa aaggatgtaa ggaaagaggc |
| 37561 | agtgttaaac actgttaaga ggacagttta tcagtatttt ttactaaact ttaataaaac |
| 37621 | ttttctattt gaatttctgc tatgaatttt tcttcagcat ttgtcctcag tacaggtggt |
| 37681 | tccttgaaac attgtttcta ataaaactag aacatcctga tattttatcc attctataga |
| 37741 | gatcattgat ggtacacaga catacagtgg attatgtttg ttgagtgaat ggaaagagag |
| 37801 | attgttaggt ttacaacgat gcagctcttg agaccggagt ttaagatcag cctgggcaac |
| 37861 | atagtgaaac cccatcttta gctgggcatg gagatggatg cctatagtcc tagctactgg |
| 37921 | ggagacgggg gcaggaggat tgcttgaacc caggagttaa cagactgcac tcagtgacag |
| 37981 | agccagactc caacacaaaa aaaaaaaaaa aaaaaaagca aattaccagt gagtagtgtg |
| 38041 | ttacttgggt ttttaatagg catcttatta acatgttcca acttgagccc ttaactttct |
| 38101 | ccacctaccc ccttccacaa acctgttttc actgtcttct ctgtcttagt taatgtcagc |
| 38161 | tttgtctgtc cagctgctca ggctaaaact tttctttcat ataacacatc ctatcagcag |
| 38221 | ctcctgtttg tgggtaggca ttttgccttt tttttttttt ttttttttaa actgctatat |
| 38281 | ctctagcatg tagaacagtg cctggcagca cataataggt gcttaatata atatttgttg |
| 38341 | aaagaacaag tcagtgagta tttttaatgt gaggtgcaaa gagaaaaaaa aatgtatctt |
| 38401 | tgaggtgtgg agttttgaag aacttccatt ttctaagcat ttgtgtaatg ttggagttac |
| 38461 | ttgttccttt tgtaatctga aagtatgctt taaaaaaaat tagtgtactt ttgagaattt |
| 38521 | tcattttgct ttctattctt ccttgctttg tgcatgttta tctagactgc tgatcttgga |
| 38581 | cttgatattg gtgcccaggg agaacccctt ggatatcgcc aggatggtat gtgtctcata |
| 38641 | tttctcgatt aactccagat caagctaaag ttctaaaact tttatcagaa gagccggttt |
| 38701 | gctcatctgg gaaaccagtg ttggcagaaa agtagtggct tcaattaaaa gcagttctta |
| 38761 | aattccagtc agcaacagta tctttaatgg agcacaggga attcagagcc acacaatgag |
| 38821 | tagcagtagg attacaccac caacaaatac atgctactgc taggcctctg cagtgcagga |
| 38881 | tgttacaatt tacctggctt tttattctct ttttggccag aggactcata atacctttgt |
| 38941 | ctacaagcta cccaaggaag ataggaaaac tcctgtttct aggctcagat ctcgggtggg |
| 39001 | tttttacata gttgcattat catcagggtt ttcttgaaaa gctaatttaa atctgggtaa |
| 39061 | tgaacatgga ggatggcata gaccactaac aattataact gtcttacatt tataaccgca |
| 39121 | tctgcttcta cctaattatg aaaccactaa agcgcagatt cttactgtga gaaataacat |
| 39181 | gtcaacccta agataaaata tgttgaggtt tcatggaaat agtgcctttc cttagtactt |
| 39241 | ttgtgggtgt cacttggcct ttttgtcaag atagattaca cctgccagac ctcattattg |
| 39301 | tcttaatcct ccttcccatg acttctcact gcctaggtgg tcacacagta gattcctgct |
| 39361 | tcttctcctc gggaacccca agtctcttga caggggtaaa tgcagagtgt tcagggttag |
| 39421 | actaatgatg tgactaggcc ctgctggtgt gcctgtctga tggaaataga tgttatttgt |
| 39481 | gtagtctcat gggtggcctg gcactgagta attacttggc taaagaaagc tggaggttga |
| 39541 | agaggctaga aagcgttgtt ttctgacaag tttgctgctg aactttggat gccctaacct |
| 39601 | cagtgttaac gtctatgtct gcttctctcc tctctctttt gccttccttc ttgcctattt |
| 39661 | tgttgacacc ctgactcttc tagatcctag ctatcgttct tttcactctg gtggatatgg |
| 39721 | ccaggatgcc ttgggtatgg accccatgat ggaacatgag atgggtggcc accaccctgg |
| 39781 | tgctgactat ccagttgatg ggctgccaga tctggggcat gcccaggacc tcatggatgg |
| 39841 | gctgcctcca ggtgacagca atcagctggc ctggtttgat actgacctgt aaatcatcct |
| 39901 | ttaggtaaga agttttaaaa agccagtttg ggtaaaatac ttttactctg cctacagaac |
| 39961 | ttcagaaaga cttggttggt agggtgggag tggtttaggc tatttgtaaa tctgccacaa |
| 40021 | aaacaggtat atactttgaa aggagatgtc ttggaacatt ggaatgttct cagatttctg |
| 40081 | gttgttatgt gatcatgtgt ggaagttatt aactttaatg ttttttgcca cagcttttgc |
| 40141 | aacttaatac tcaaatgagt aacatttgct gttttaaaca ttaatagcag cctttctctc |
| 40201 | tttatacagc tgtattgtct gaacttgcat tgtgattggc ctgtagagtt gctgagaggg |
| 40261 | ctcgaggggt gggctggtat ctcagaaagt gcctgacaca ctaaccaagc tgagtttcct |
| 40321 | atgggaacaa ttgaagtaaa ctttttgttc tggtcctttt tggtcgagga gtaacaatac |
| 40381 | aaatggattt tgggagtgac tcaagaagtg aagaatgcac aagaatggat cacaagatgg |
| 40441 | aatttatcaa accctagcct tgcttgttaa attttttttt tttttttttt aagaatatct |
| 40501 | gtaatggtac tgactttgct tgctttgaag tagctctttt tttttttttt tttttttttt |
| 40561 | tgcagtaact gttttttaag tctctcgtag tgttaagtta tagtgaatac tgctacagca |
| 40621 | atttctaatt tttaagaatt gagtaatggt gtagaacact aattcataat cactctaatt |
| 40681 | aattgtaatc tgaataaagt gtaacaattg tgtagccttt ttgtataaaa tagacaaata |
| 40741 | gaaaatggtc caattagttt cctttttaat atgcttaaaa taagcaggtg gatctatttc |
| 40801 | atgtttttga tcaaaaacta tttgggatat gtatgggtag ggtaaatcag taagaggtgt |
| 40861 | tatttggaac cttgttttgg acagtttacc agttgccttt tatcccaaag ttgttgtaac |
| 40921 | ctgctgtgat acgatgcttc aagagaaaat gcggttataa aaaatggttc agaattaaac |
| 40981 | ttttaattca ttcgattg |

Noggin (Nog)

The NOG gene encodes the noggin protein, and is a bone morphogenetic protein 4 (BMP4) inhibitor. Activation of NOG in supporting cells inhibits BMP4 and induces hair cell regeneration (Lewis et al. (2018) Hear Res. 364:1-11).

The human NOG gene is located on chromosome 17q22. It contains 1 exon encompassing ˜2 kilobases (kb) (NCB1 Accession No. NG_011958.1). The full-length wildtype NOG protein expressed from the human NOG gene is 232 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype NOG protein is or includes the sequence of SEQ ID NO: 16. Non-limiting examples of a nucleic acid encoding a wildtype NOG protein is or includes SEQ ID NO: 19. As can be appreciated in the art, at least some or all of the codons in SEQ ID NO: 19 can be codon-optimized to allow for optimal expression in a non-human primate.

Human Full-length Wildtype NOG Protein
(SEQ ID NO: 16)
MERCPSLGVTLYALVVVLGLRATPAGGQHYLHIRPAPSDNLPLVDLIEH
PDPIFDPKEKDLNETLLRSLLGGHYDPGFMATSPPEDRPGGGGGAAGGA
EDLAELDQLLRQRPSGAMPSEIKGLEFSEGLAQGKKQRLSKKLRRKLQM
WLWSQTFCPVLYAWNDLGSRFWPRYVKVGSCFSKRSCSVPEGMVCKPSK
SVHLTVLRWRCQRRGGQRCGWIPIQYPIISECKCSC
Mouse Full-length Wildtype NOG Protein
(SEQ ID NO: 17)
MERCPSLGVTLYALVVVLGLRAAPAGGQHYLHIRPAPSDNLPLVDLIEH
PDPIFDPKEKDLNETLLRSLLGGHYDPGFMATSPPEDRPGGGGGPAGGA
EDLAELDQLLRQRPSGAMPSEIKGLEFSEGLAQGKKQRLSKKLRRKLQM
WLWSQTFCPVLYAWNDLGSRFWPRYVKVGSCFSKRSCSVPEGMVCKPSK
SVHLTVLRWRCQRRGGQRCGWIPIQYPIISECKCSC
Rat Full-length Wildtype NOG Protein
(SEQ ID NO: 18)
MERCPSLGVTLYALVVVLGLRAAPAGGQHYLHIRPAPSDNLPLVDLIEH
PDPIFDPKEKDLNETLLRSLLGGHYDPGFMATSPPEDRPGGGGGPAGGA
EDLAELDQLLRQRPSGAMPSEIKGLEFSEGLAQGKKQRLSKKLRRKLQM
WLWSQTFCPVLYAWNDLGSRFWPRYVKVGSCFSKRSCSVPEGMVCKPSK
SVHLTVLRWRCQRRGGQRCGWIPIQYPIISECKCSC
Human Wildtype NOG cDNA
(SEQ ID NO: 19)
atggagcgctgccccagcctaggggtcaccctctacgccctggtggtgg
tcctggggctgcgggcgacaccggccggcggccagcactatctccacat
ccgcccggcacccagcgacaacctgcccctggtggacctcatcgaacac
ccagaccctatctttgaccccaaggaaaaggatctgaacgagacgctgc
tgcgctcgctgctcgggggccactacgacccaggcttcatggccacctc
gccccccgaggaccggcccggcgggggcgggggtgcagctgggggcgcg
gaggacctggcggagctggaccagctgctgcggcagcggccgtcggggg
ccatgccgagcgagatcaaagggctagagttctccgagggcttggccca
gggcaagaagcagcgcctaagcaagaagctgcggaggaagttacagatg
tggctgtggtcgcagacattctgccccgtgctgtacgcgtggaacgacc
tgggcagccgcttttggccgcgctacgtgaaggtgggcagctgcttcag
taagcgctcgtgctccgtgcccgagggcatggtgtgcaagccgtccaag
tccgtgcacctcacggtgctgcggtggcgctgtcagcggcgcgggggcc
agcgctgcggctggattcccatccagtaccccatcatttccgagtgcaa
gtgctcgtgctag

A non-limiting example of a human wildtype NOG genomic DNA sequence is SEQ ID NO: 20. The exons in SEQ ID NO: 20 are: nucleotide positions 1-1892 (exon 1).

| Human Wildtype NOG Gene | |
|---|---|
| (SEQ ID NO: 20) | |
| 1 | aaaccggtgc caacgtgcgc ggacgccgcc gccgccgccg ccgctggagt ccgccgggca |
| 61 | gagccggccg cggagcccgg agcaggcgga gggaagtgcc cctagaacca gctcagccag |
| 121 | cggcgcttgc acagagcggc cggacgaaga gcagcgagag gaggagggga gagcggctcg |
| 181 | tccacgcgcc ctgcgccgcc gccggcccgg gaaggcagcg aggagccggc gcctcccgcg |
| 241 | ccccgcggtc gccctggagt aatttcggat gcccagccgc ggccgccttc cccagtagac |
| 301 | ccgggagagg agttgcggcc aacttgtgtg cctttcttcc gccccggtgg gagccggcgc |
| 361 | tgcgcgaagg gctctcccgg cggctcatgc tgccggccct gcgcctgccc agcctcgggt |
| 421 | gagccgcctc cggagagacg ggggagcgcg gcggcgccgc gggctcggcg tgctctcctc |
| 481 | cggggacgcg ggacgaagca gcagccccgg gcgcgcgcca gaggcatgga gcgctgcccc |
| 541 | agcctagggg tcaccctcta cgccctggtg gtggtcctgg ggctgcgggc gacaccggcc |
| 601 | ggcggccagc actatctcca catccgcccg gcacccagcg acaacctgcc cctggtggac |
| 661 | ctcatcgaac acccagaccc tatctttgac cccaaggaaa aggatctgaa cgagacgctg |
| 721 | ctgcgctcgc tgctcggggg ccactacgac ccaggcttca tggccacctc gccccccgag |
| 781 | gaccggcccg gcgggggcgg gggtgcagct gggggcgcgg aggacctggc ggagctggac |
| 841 | cagctgctgc ggcagcggcc gtcgggggcc atgccgagcg agatcaaagg gctagagttc |
| 901 | tccgagggct tggcccaggg caagaagcag cgcctaagca agaagctgcg gaggaagtta |
| 961 | cagatgtggc tgtggtcgca gacattctgc cccgtgctgt acgcgtggaa cgacctgggc |
| 1021 | agccgctttt ggccgcgcta cgtgaaggtg ggcagctgct tcagtaagcg ctcgtgctcc |
| 1081 | gtgcccgagg gcatggtgtg caagccgtcc aagtccgtgc acctcacggt gctgcggtgg |
| 1141 | cgctgtcagc ggcgcggggg ccagcgctgc ggctggattc ccatccagta ccccatcatt |
| 1201 | tccgagtgca agtgctcgtg ctagaactcg ggggccccct gcccgcaccc ggacacttga |
| 1261 | tcgatcccca ccgacgcccc ctgcaccgcc tccaaccagt tccaccaccc tctagcgagg |
| 1321 | gttttcaatg aacttttttt tttttttttt tttttttttc tgggctacag agacctagct |
| 1381 | ttctggttcc tgtaatgcac tgtttaactg tgtaggaatg tatatgtgtg tgtatatacg |
| 1441 | gtcccagttt taatttactt attaaaaggt cagtattata cgttaaaagt taccggcttc |
| 1501 | tactgtattt ttaaaaaaaa gtaagcaaaa gaaaaaaaaa agaacagaga aaagagagac |
| 1561 | ttattctggt tgttgctaat aatgttaacc tgctatttat attccagtgc ccttcgcatg |
| 1621 | gcgaagcagg ggggaaaagt tatttttttc ttgaagtaca aagagacggg ggaacttttg |
| 1681 | tagaggactt tttaaaagct attttccatt cttcggaaag tgttttggtt ttccttggac |
| 1741 | ctcgaagaag ctatagagtt caatgttatt ttacagttat tgtaaatata gagaacaaat |
| 1801 | ggaatgacta atcattgtaa attaagagta tctgctattt attctttata atatcccgtg |
| 1861 | tagtaaatga gaaagaagtg cagagcagga tt |

Growth Factor Independent 1 Transcriptional Repressor (GFI-1)

The GFI-1 gene encodes a nuclear zinc finger protein, and acts as a transcriptional repressor. GFI-1 is activated by Atoh1 and Pou4f3 in early development and is required for hair cell survival after birth (Hertzano et al. (2004) Hum. Mol. Genet. 13(18):2143-2153; Costa et al. (2015) Genom Data 6:77-80).

The human GFI-1 gene is located on chromosome 1p22. It contains 7 exons encompassing ˜12 kilobases (kb) (NCBI Accession No. NG_007874.1). The full-length wildtype GFI-1 protein expressed from the human GFI-1 gene is 422 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype GFI-1 protein is or includes the sequence of SEQ ID NO: 21. Non-limiting examples of a nucleic acid encoding a wildtype GFI-1 protein is or includes SEQ ID NO: 24. As can be appreciated in the art, at least some or all of the codons in SEQ ID NO: 24 can be codon-optimized to allow for optimal expression in a non-human primate.

Human Full-length Wildtype GFI-1 Protein
(SEQ ID NO: 21)
MPRSFLVKSKKAHSYHQPRSPGPDYSLRLENVPAPSRADSTSNAGGAKA
EPRDRLSPESQLTEAPDRASASPDSCEGSVCERSSEFEDFWRPPSPSAS
PASEKSMCPSLDEAQPFPLPFKPYSWSGLAGSDLRHLVQSYRPCGALER
GAGLGLFCEPAPEPGHPAALYGPKRAAGGAGAGAPGSCSAGAGATAGPG
LGLYGDFGSAAAGLYERPTAAAGLLYPERGHGLHADKGAGVKVESELLC
TRLLLGGGSYKCIKCSKVFSTPHGLEVHVRRSHSGTRPFACEMCGKTFG
HAVSLEQHKAVHSQERSFDCKICGKSFKRSSTLSTHLLIHSDTRPYPCQ
YCGKRFHQKSDMKKHTFIHTGEKPHKCQVCGKAFSQSSNLITHSRKHTG
FKPFGCDLCGKGFQRKVDLRRHRETQHGLK
Mouse Full-length Wildtype GFI-1 Protein
(SEQ ID NO: 22)
MPRSFLVKSKKAHSYHQPRSPGPDYSLRLETVPAPGRAEGGAVSAGESK
MEPRERLSPDSQLTEAPDRASASPNSCEGSVCDPCSEFEDFWRPPSPSV
SPASEKSLCRSLDEAQPYTLPFKPYAWSGLAGSDLRHLVQSYRQCSALE
RSAGLSLFCERGSEPGRPAARYGPEQAAGGAGAGQPGRCGVAGGATSAA
GLGLYGDFAPAAAGLYERPSTAAGRLYQDHGHELHADKSVGVKVESELL
CTRLLLGGGSYKCIKCSKVFSTPHGLEVHVRRSHSGTRPFACEMCGKTF
GHAVSLEQHKAVHSQERSFDCKICGKSFKRSSTLSTHLLIHSDTRPYPC
QYCGKRFHQKSDMKKHTFIHTGEKPHKCQVCGKAFSQSSNLITHSRKHT
GFKPFGCDLCGKGFQRKVDLRRHRETQHGLK
Rat Full-length Wildtype GFI-1 Protein
(SEQ ID NO: 23)
MPRSFLVKSKKAHSYHQPRSPGPDYSLRLETVPVPGRADGGAVSAGESK
MEPRERLSPESQLTEAPDRASASPNSCEGSVCDPSSEFEDYWRPPSPSV
SPASEKSLCRSLDEAQPYTLPFKPYAWSGLAGSDLRHLVQSYRQCSALE
RSAGLSLFCERGAESGRPAARYGSEQAAGGAGAGQPGSCGAASGATSAG
GLGLYGDFAPAAAGLFERPSTAAGRLYQDRGHELHADKSVGVKVESELL
CTRLLLGGGSYKCIKCSKVFSTPHGLEVHVRRSHSGTRPFACEMCGKTF
GHAVSLEQHKAVHSQERSFDCKICGKSFKRSSTLSTHLLIHSDTRPYPC
QYCGKRFHQKSDMKKHTFIHTGEKPHKCQVCGKAFSQSSNLITHSRKHT
GFKPFGCDLCGKGFQRKVDLRRHRETQHGLK
Human Wildtype GFI-1 cDNA
(SEQ ID NO: 24)
atgccgcgctcatttctcgtcaaaagcaagaaggctcacagctaccacc
agccgcgctccccaggaccagactattccctccgtttagagaatgtacc
ggcgcctagccgagcagacagcacttcaaatgcaggcggggcgaaggcg
gagccccgggaccgtttgtcccccgaatcgcagctgaccgaagccccag
acagagcctccgcatccccagacagctgcgaaggcagcgtctgcgaacg
gagctcggagtttgaggacttctggaggcccccgtcaccctccgcgtct
ccagcctcggagaagtcaatgtgcccatcgctggacgaagcccagccct
tccccctgcctttcaaaccgtactcatggagcggcctggcgggttctga
cctgcggcacctggtgcagagctaccgaccgtgtggggccctggagcgt
ggcgctggcctgggcctcttctgcgaacccgccccggagcctggccacc
cggccgcgctgtacggcccgaagcgggctgccggcggcgcgggggccgg
ggcgccagggagctgcagcgcaggggccggtgccaccgctggccctggc
ctagggctctacggcgacttcgggtctgcggcagccgggctgtatgaga
ggcccacggcagcggcgggcttgctgtaccccgagcgtggccacgggct
gcacgcagacaagggcgctggcgtcaaggtggagtcggagctgctgtgc
acccgcctgctgctgggcggcggctcctacaagtgcatcaagtgcagca
aggtgttctccacgccgcacgggctcgaggtgcacgtgcgcaggtccca
cagcggtaccagaccctttgcctgcgagatgtgcggcaagaccttcggg
cacgcggtgagcctggagcagcacaaagccgtgcactcgcaggaacgga
gctttgactgtaagatctgtgggaagagcttcaagaggtcatccacact
gtccacacacctgcttatccactcagacactcggccctacceptgtcag
tactgtggcaagaggttccaccagaagtcagacatgaagaaacacactt
tcatccacactggtgagaagcctcacaagtgccaggtgtgcggcaaggc
attcagccagagctccaacctcatcacccacagccgcaaacacacaggc
ttcaagcccttcggctgcgacctctgtgggaagggtttccagaggaagg
tggacctccgaaggcaccgggagacgcagcatgggctcaaatga

A non-limiting example of a human wildtype GFI-1 genomic DNA sequence is SEQ ID NO: 25. The exons in SEQ ID NO: 25 are: nucleotide positions 1-151 (exon 1), nucleotide positions 3291-3504 (exon 2), nucleotide positions 3831-4013 (exon 3), nucleotide positions 5789-6276 (exon 4), nucleotide positions 6392-6529 (exon 5), nucleotide positions 8124-8289 (exon 6), and nucleotide positions 10670-12116 (exon 7). The introns in SEQ ID NO: 25 are: nucleotide positions 152-3290 (intron 1), nucleotide positions 3505-3830 (intron 2), nucleotide positions 4014-5788 (intron 3), nucleotide positions 6277-6391 (intron 4), nucleotide positions 6530-8123 (intron 5), and nucleotide position 8290-10669 (intron 6).

| Human Wildtype GFI-1 Gene | |
|---|---|
| (SEQ ID NO: 25) | |
| 1 | gagggtgcgc ccaccggtcc cgccgggcgc ccgcgggacg cgccgccagg gccctctccg |
| 61 | ccgggggctc ggcgctcgcc cacctcttcc aaatttaacc attacctaaa tccgaaggga |
| 121 | aatgagcaaa cctctcggat tgggtgtcaa ggtattttca gcctcgttgg gcgtatttat |
| 181 | ccccaagtgt ttccacaaca agctatttcg gggcctgcgg ggcaggtttc gctctgcgga |
| 241 | cgccgtggcc actcgccggg ctccaggccg gcggcaccgc gggccggtga ttcacggtcc |
| 301 | cgacccgggg gtggtgcagc cctaggaggc ggcggggtcg ggggtggggg gggcgggtga |
| 361 | ccgaggcctg aggggcgggg agggtcctcg gagcggggcg ccccccaccc ctctctcgcc |
| 421 | agtcaatctg tgtcctcaat ctgtggcttc tctcgctgcg gaagtctccc tggagccaag |
| 481 | aatagttcat tttctttcaa gtcatttcta gtgcctaagt gtccggacct ccaatttccc |
| 541 | ccatcccctg ccgacccaca gggagagaac tgggaggact actaaggggc gcgcgggacg |
| 601 | ggctggaaag gccaggcccc ccaccgcctg gccacttgcg caaaggagcg cgcccggccg |
| 661 | cccgacgggg gttgggagca ggtctgggag ggctatgcga gcgactcagt aacgctcagg |
| 721 | aagtgaagct tgtggttttg ggggctgagc tcggaaggag aatttttttt ttttttaagt |
| 781 | cagagagata gagcggtctc tcccgaaagc aagattccgt ttgaaaactc tcctagcgcg |
| 841 | gtgcccgcgc cgtgactccg caggtaggtc cgccgagcct gttctgcgcc tcctgccctg |
| 901 | gtgggggcgg ccgcggggac tcgcagagca ctggcactgc gggggcgatc agagggcgcg |
| 961 | ggcggtttcc cacctgctgc ggaccgccgt gcgggagccc agagagctcc ggcagctggg |
| 1021 | ttagggcgcg acccgcgcag tgtgactgga aatctggagc tgggggcgcg cagcaggcgg |
| 1081 | tctggtggtt cggcagggga gccaaatcca ccagggaagg aaacatctgg tggggaggcg |
| 1141 | gcggcagctg cgctcgggag gacgccccct tagcgccctc ggctccctcc ttcctgggcc |
| 1201 | cggacggtga ggagaggcct gagcgcgcgg aggggccgcc ccacctcccg cgccagctgc |
| 1261 | agcgcggggt tgccttccca cgcgctcggc ccaggccccg gggcccctat ctcctccaaa |
| 1321 | ctctgtcgct ccccacagga accagcaaag cgggccgggg tgcgagagag gcgtgggctt |
| 1381 | acagcccggg tggggaggcg gcctccgcgt ccgcctggtc tctggtggcg ccagcccaga |
| 1441 | cccagctccg gcgctgacag ttaccccgcc cccatctgtc ccgctcccag ccaacgtggg |
| 1501 | tccaagctgc agcgggaccc tcgggacaac gccactccgt ttttcttttc ttccgagttt |
| 1561 | cgtggctgtt taaagaattg ggtttggggt ttgtggcgtc taattgtacg gacgagaagt |
| 1621 | gcaggaagcg acaaagctct agccctagga gccaccccgg agggaggcgg atggcactct |
| 1681 | cacccctagg ggcattctgg cgcttgggta gcgggaaact tcgcgggagc cccgcgacac |
| 1741 | gtcccaggcg tcttttctcc caggtctatt cccattcctc cggagaaggg gacacaatgg |
| 1801 | ggctggggat ctggagcagg gggcctgcac cctacaggga ccaaggcctg taggactcgt |
| 1861 | ttgagctgag agcgccaacg gacagacgta gactgtgtgg cctgcatctt gcctaggaag |
| 1921 | ccgaggggct cctagtccgg cagtggaaac agcgcgaagc cggaggactg caggtcctgc |
| 1981 | cccggcccag agttcccagc accctcgttt ctgaaccagc cgaggccacg gagaactgct |
| 2041 | gtactgcagc tcacgtgtga acccggtcac catcgccttc accccgggag gaaggcagat |
| 2101 | tcgtttactc cagaccacct cgactgtggg gtaccgcccc cggagccggc tggagcctag |
| 2161 | cggcaggcgc agccacgccc tcccgctgcg ctcagatttc gacctggtat taggtgaact |
| 2221 | gattgggggt taatgagagc gacgccccgg gcagctagtt ccctcccggg cccgggcccg |
| 2281 | acccccgctc tctgctaatg cagcctgcgc gctctggcgt cctgtctttt ttgtctgcta |
| 2341 | aattgtcggt gcactaccga ctcgggacac ctagcatttc ccagtcaacg ttcgtggatc |
| 2401 | gggctccacc tccctaggac aagatttttt ggtgagcaga acggaaagtg cttttttccc |
| 2461 | gggacctgat tcccgaggtt aggtctccat ggtctgggat ggctcgccgc agcctcgacc |
| 2521 | ggtgcccgcc gcagccggga gtccaagggc aaagtttctc ctacgtgggg cactagtgag |
| 2581 | gggcgagtgg gatcacccag atgcgagttt ctcctggggc gggggttggt cgtctgttgt |
| 2641 | tcccctcact ttcctgtccc tttgctctcc cacctccttt ctctggcctc tgctgtcccc |
| 2701 | aatccctctg ctgctgtcct cccgccgccc cacagtttta tcttgtgttc tgttcccccc |
| 2761 | tccccccggt cctttcactc cagttggtag ctggctcttg gaggtcttgg ctccttgccc |
| 2821 | cttccgggtc ctcgaccact gggcatcccc ggcccctaaa ccgatccgcg tgtccccgcc |
| 2881 | ctccctcgcc agccgtaaag cacagccagg caggcgatga gtagctgagt tggggtaacc |
| 2941 | cacccgatgg gaactacagc tctccaggga gtttgattgc cggagcgagc ttcgctagga |
| 3001 | aaggggagga gctggggggc gtgggcaggg aggaggaaag gggcctgaga cagggccccg |
| 3061 | ggacaggttt taccgctgag ctgtgtcagt ggcggcggcg gcaacgacgg cgggttcgcg |
| 3121 | ccacctgtcc aagtgccacc tggtaagcgc ggcgcagcag ggtcaagccc ctcctcccgt |
| 3181 | gggccctctg cgcgcctccc tggcccgcgc tctccctccg cctgggtgcc cagtccgccg |
| 3241 | caccggagag tactgaccca cgtctccacc tggttttctt tccctctcag gtctcctccg |
| 3301 | ggctggggct gagcaagccc tcggagtgac cgtgggtgac agcggctcca gggactcttg |
| 3361 | gggcgcagtg gggaaagtgc cggaccacca tgccgcgctc atttctcgtc aaaagcaaga |
| 3421 | aggctcacag ctaccaccag ccgcgctccc caggaccaga ctattccctc cgtttagaga |
| 3481 | atgtaccggc gcctagccga gcaggtgcga ggcgcgcgcg ggccaggcgg ggctgctccc |
| 3541 | ccggatgcct actgcacctc ggcacaccat tagtccggag ctgggagggg ctgccccaac |
| 3601 | gtcccttttg ctgctgtttt tgtttcctac tgtcctggtt cctccgggtt tgtctcctag |
| 3661 | gtgccatggc ctctctgcgc ctgccctcgg atccgagagg gttcccggcc ggggtctggg |
| 3721 | tggagagggg aagacgctcg gctgccctgg tcgggggatt gggggagcct tcagcaccct |
| 3781 | cagactcaac cggtcccagc ctgagcccct cacctgcctc ctctttgcag acagcacttc |
| 3841 | aaatgcaggc ggggcgaagg cggagccccg ggaccgtttg tcccccgaat cgcagctgac |
| 3901 | cgaagcccca gacagagcct ccgcatcccc agacagctgc gaaggcagcg tctgcgaacg |
| 3961 | gagctcggag tttgaggact tctggaggcc cccgtcaccc tccgcgtctc caggtaggaa |
| 4021 | cccactggga acctcttggg cgggagctgc agggacccgg cagtgctggg ggggaattgg |
| 4081 | cgcgaccttg ggcgtagaaa tgctaacggg gagttggaga gtctttccgg gagaagggag |
| 4141 | ctgattcgta ggggaaggag gcatccggct tctctgggac ttggacagct tgcccgctgg |
| 4201 | ggctgctgcc tccatcccag gcggcaggac cctagctgct tgtcgcttag attcgtttgc |
| 4261 | gcggagctgg ccagtgacgg aaaacaaacc agtcgtttcg actggcggca acgctgacct |
| 4321 | ttcattttct gaccagacct gactgtttta taaagtgcta ggatcctgca atctagaccc |
| 4381 | ccaaacctca aacagagaac agggcagaac gggccaggca gaggagctag gcgctgggcg |
| 4441 | gcagggaggg ggcaggacga aaatctcagc ccgcggcttg gtcttcacag gcgcagattg |
| 4501 | ggggcctgtt tcatttttcg ttttgccggg ttaacctagc ctcaggggcc tgctctctgg |
| 4561 | gtttcatttc cagcgagcaa tccagcttca ggcaaactaa gtgaccacac gttgggtggg |
| 4621 | ggcgtctcga gtcccggccg ggggaaggaa tgagcagacc agccggattc tgtcaagggc |
| 4681 | cggttatatc cagaatatgt ttgctagttt tagaagatac caccacccgt cccacaatca |
| 4741 | gtgagttgac ttggcgaaaa ccatagctcc agcaagtgtg tctgggagcc ggcggcggga |
| 4801 | ggattcttcc tgccagggcg tcaagtggcc agacaaggat tgggcgcgcc ccgaacccct |
| 4861 | ccgaacgaaa ctccgggtac agcctctcac tgaagtggcc agcctgaact ggagtgttgt |
| 4921 | gcgcacacac acacacacac atttgtaaat gccgtatgca ctcacatgcg ttggggtcac |
| 4981 | tagttttagc aaaattcacg tgggtggggg cgtagcaggc cgagaattca gagctgtctc |
| 5041 | cttgcaggtg gtggctaaac cttatgagtt atagttattc tctgagaaat tcaggttccc |
| 5101 | cgcctccatc aaactgtaac aggaatgggg agtatttggc tgtcaattta agcccaaaag |
| 5161 | cccctttcct gctgctcctt tgctacgtac actgggcact taacttcgtg aaatcttaat |
| 5221 | accttcgggt ttattcagac agcagccttt cgggtagttc ggggccgcat ttatggacct |
| 5281 | tctccctcct tcctcttgga ttctgggaag aaaaagaatt gaatgggaac atgtaggggc |
| 5341 | tgggagagtg cctgcgctgg tggctggacc cttccgccct tgagtgctgt gaggggccga |
| 5401 | acggccgcca ccttctcctt cttaacagct caactacggg catttataga tgcgcccttc |
| 5461 | cctgtaggat ctccaggtgc gcgggtccag ccagaaaaga tcctcggaac gccgagcgcc |
| 5521 | tccgctgcac tcgcacagaa tttacgacct cctctcccga ggtcttttca atgatctgtt |
| 5581 | tactgttctg cctcctatag tggcctgcga ggccccaggg cccgggccac gttttaccct |
| 5641 | ggggcgagcc tggcacctgg cgcacgcagt gttctacaag cgctgggtgc cccgcagtcc |
| 5701 | gcgaacacgc cacgctcgca gccgcagccc ggcggcctcc gctctgccgt ctgaagcctg |
| 5761 | accggacgct ccccttgtgc ctccacagcc tcggagaagt caatgtgccc atcgctggac |
| 5821 | gaagcccagc ccttccccct gcctttcaaa ccgtactcat ggagcggcct ggcgggttct |
| 5881 | gacctgcggc acctggtgca gagctaccga ccgtgtgggg ccctggagcg tggcgctggc |
| 5941 | ctgggcctct tctgcgaacc cgccccggag cctggccacc cggccgcgct gtacggcccg |
| 6001 | aagcgggctg ccggcggcgc gggggccggg gcgccaggga gctgcagcgc aggggccggt |
| 6061 | gccaccgctg gccctggcct agggctctac ggcgacttcg ggtctgcggc agccgggctg |
| 6121 | tatgagaggc ccacggcagc ggcgggcttg ctgtaccccg agcgtggcca cgggctgcac |
| 6181 | gcagacaagg gcgctggcgt caaggtggag tcggagctgc tgtgcacccg cctgctgctg |
| 6241 | ggcggcggct cctacaagtg catcaagtgc agcaaggtga ggctcccgag ctcaccacct |
| 6301 | cgcctgccgt gcgcccgctt cccctacccg cgcctcgcct gcgccccgcg gcccctctca |
| 6361 | gcggccttct ctctggcccc acccgcctta ggtgttctcc acgccgcacg ggctcgaggt |
| 6421 | gcacgtgcgc aggtcccaca gcggtaccag accctttgcc tgcgagatgt gcggcaagac |
| 6481 | cttcgggcac gcggtgagcc tggagcagca caaagccgtg cactcgcagg taagcgcggg |
| 6541 | gcgcaccgcc gcgcgcggcc ctgctcgggg atcttctgca tctcctcggt gcagcaccag |
| 6601 | ccactctctg cctggaagtt ttctcctcga cttcccccag tttcctcccc caagccctcc |
| 6661 | gctgcgtccc cttgccctgg tgcaggtgtg tagggaaagg aggattgtgg ccggctcagg |
| 6721 | ccttgaggca gccctggatt ttggtgtcac accactgtga gcctcgagag tgtgatcctc |
| 6781 | attgttactt tgggcttgag gtaggtttgt atgcactgat tcgtgctgct gatatatcag |
| 6841 | acttactagc tctgtttctt tgtgcctatt cttttcacca aatggttgtc acttaatttg |
| 6901 | cattgacccc tctcgactga aaaggcagga atctcagctc atttagagca tctagtagca |
| 6961 | tattcacccc gctattcatt ctttccttcc ttcctttctt ttcttttctt ttttcttttc |
| 7021 | ttttctttca gagtctggct ctgttgccca ggctggaggg cagttgcaca atctcagctc |
| 7081 | actgcaacct ccccctgtca ggttaaagtg attctcgtgc ctcagcctcc taagtagctg |
| 7141 | ggattacagg cgcatgccac tacagcacag ctaatttttg tgttcttagt agagacggga |
| 7201 | tttcgccacg ttagccaggc tggtcacgaa ctcctggcct caagtgatcc accagcctgg |
| 7261 | gcctcccaaa gtgctgggat tacaggcgtg agccaccatg ccccaccgcc gctatttatt |
| 7321 | cattcattca ttaataaata tttgttggct aacttccagg tgccaagtac ttaagaatct |
| 7381 | tataacacat caggtccttg acagcatgcc cacatgaaga ttatagttta gctgagagat |
| 7441 | ggagagtaga tgagcaagta aatatgccaa tagctatctc aggagaatgc ctacttacga |
| 7501 | aggctaaaaa gagtattagc ccatctcccc cagcacccac actggctggg gggaggtggc |
| 7561 | atctcaagtg actgaggtct aagcctcctg ttgaggaggg tggagaagtg tgtgctaatg |
| 7621 | ggtgtcaaaa aaagcagggt gtggatatgt atttgccatg gggtgtggaa ggttgtgggt |
| 7681 | gaagaatgtt ttggtagaaa aagtgttgaa gggccaggca cggtggctca cgcctgtaat |
| 7741 | cccaatactt tgagaggccg aggtgggcga atcacttgag gccaggtgtt tgagaccagc |
| 7801 | ctgaccaaca tggtgaaacc ccatctctac taaaaataca aaaattagtc aggtgtgatg |
| 7861 | gcgtgtgcct gtagtccctg ctacttggga ggctgagaca cgagaattgc ttgaacctgg |
| 7921 | gaggtggagg gtgcagtgaa ctgagatcgt tccactgcac tcaagcctgg gcgacagagg |
| 7981 | agactgtctc aaaaaaagaa agaaaaagtg ttcaagggat tttagggtca gctgaggggt |
| 8041 | gaggagagca gcagtctagt tgactgcagt aggagttctg catctctctc tctctctctc |
| 8101 | tctctctctc tctctctctg caggaacgga gctttgactg taagatctgt gggaagagct |
| 8161 | tcaagaggtc atccacactg tccacacacc tgcttatcca ctcagacact cggccctacc |
| 8221 | cctgtcagta ctgtggcaag aggttccacc agaagtcaga catgaagaaa cacactttca |
| 8281 | tccacactgg tgagctaaaa aggcccttgg cttgtaggaa acaccctgag gccaacatta |
| 8341 | ctcatcttct ctgatttctg gccccagtga gtggtggatg aggcctttct gatggagtta |
| 8401 | ttctctgctc tgtgttaaag aaaacaaagg ggtgggttct ttggttcatt taccggcata |
| 8461 | attctcccca gagccacctt gatttggggt tgtgtctgaa aggccactca gcaggtcagc |
| 8521 | tcacaggtac tctatacttg gaaagaacat tttcctttag gttagcagct gcttcccctg |
| 8581 | ctgcctgctc tgggtgaaat atgaagctcc agggtcctct tagagagttg ctctaaagct |
| 8641 | tacctagaga ttgaggactt tccctaacca cctggccttt tgtgggaggg actcgtgtgg |
| 8701 | actctccggc tgcattttca ggagtctgag agcttattct gattgaagag gaacaaataa |
| 8761 | tggcaaatat gattaaactc tctgctaagc attttttaaa tgcattattt cattttatgc |
| 8821 | tcacaacaac tctgagaggt agcgactact ccttctcccc attttagaga tgaaaaaaat |
| 8881 | gaggctaggt aatctgccca gggtcacact gctagcaaat gacagggcca gagctcaaat |
| 8941 | tcaggtctga cctctcaaat gttcactctt gaccactgtt tattgtattt tatgttcaga |
| 9001 | gtcatgaggt tggtagacag aaagcttctg ttcacttatt gcccttttca aaatatctgc |
| 9061 | aagttaatgc cataataatg ataattcctt acctattata atgctttata atttacaaag |
| 9121 | tactttcaaa tctatcattt catttgattc ttattgccac tcaagaaagt agaaggagct |
| 9181 | gctcttacca tcctgaaact cagaaagagt gaatgattta tcagaagtaa gactgaatga |
| 9241 | ataatgtagc catgtaatgc tctggctttt aatcctggac tgtttgtcta acactttatg |
| 9301 | tgcgggtggg agttttaatg ccaagaacac tctaatagtc aaaagacatt tacatgagac |
| 9361 | ccagaatttc tgaaaatttt attgcagaat atgaatactg atttagaaca aatcacagtg |
| 9421 | tattctaaac acccaccctt gatgtttata aatatacttg ggtaatgtat atatttccat |
| 9481 | tgaaaaccca gaaaagtatt ctactttaat cattccctct tacctgaaat ttccatgtaa |
| 9541 | ttcactcctt ataagtaagg tattcaggac acttatcaaa atgcaactag gatcttgact |
| 9601 | gaataaaaca ttaagccctt atcaaacatt tacgttatac ctagaatttg ttttctcaga |
| 9661 | tttgtttgac cctaaaggga tagaatacat tttgatgggt ggtttcttat caaggaaatc |
| 9721 | tgaagcatga aaacagaaaa gagtttttag caaggaggac agagggttcc tcaaaaacaa |
| 9781 | acttcatcta ttttatactt tttccaaggc tgagccctga ctataatgcc atgctgggct |
| 9841 | attggaaatt catgccattt acccaacaac acatgagatg gggaacaaga caaaaccttc |
| 9901 | ttgtgttctc ttatttatta atttgtggtg aagaattgct ggtatataaa gaatcatgtg |
| 9961 | attaacccca taaaattaag gaaaaatcaa gacagtaaag tatcagctgc cttaatcctt |
| 10021 | tgtggcccaa atgtggattt ttaaaataag atattgaaaa acgtatcctg cacatgtacc |
| 10081 | ccggaactta gaaagaaaga gagagagaga gagaaataaa gaaagaaaga aagtccatgt |
| 10141 | taagatgttt tttcagatat aatctgctgt ccttcaagaa caagaaagaa gacgggctca |
| 10201 | ctgatccata caaactaaca cccacttgga aattcagatt tgaaaacttc ctctgaatta |
| 10261 | gaacggagtc acacggtttt aggacagctt ccccctcccc ttcctgttga acatctgctc |
| 10321 | tgagtgttca tggcttataa agtcagggga gtcctcccgg ggtagattca gctggggagg |
| 10381 | gcacgtggcc tttgctctgt ttccgtttag caggaaaccg tttgaggcct ttggctggga |
| 10441 | accccccttc agaaagtctc cctttcacct ggtgccccca tggtgcttcc agggactcgc |
| 10501 | attgcaggct gggagtcagt tcaggttgca acacgtcacc ctccaagttg cttgaaggcc |
| 10561 | ttagactgtg gtgcaaccag ctgctgccaa gagcatgtgg gtcacagtgg gtcccctcta |
| 10621 | gctttatcat agactcatac tttctcccct ccccctccca tccccacagg tgagaagcct |
| 10681 | cacaagtgcc aggtgtgcgg caaggcattc agccagagct ccaacctcat cacccacagc |
| 10741 | cgcaaacaca caggcttcaa gcccttcggc tgcgacctct gtgggaaggg tttccagagg |
| 10801 | aaggtggacc tccgaaggca ccgggagacg cagcatgggc tcaaatgagc accctggctg |
| 10861 | gctgcaagca gcagctacac aacactacag agggcagcct ccctgcttgc caccactctg |
| 10921 | ctccctgctt gcctccactc ccttctgact ttccagaccc caggtccagt ctgcagatcc |
| 10981 | taccaggttg ctcctccttc gccttacctc ctggagctgc cagaagaaat gaggtacctt |
| 11041 | ttcaaagtgc agccgagagt gagaaccaag tgactctcta ggcttcggac acaaataggc |
| 11101 | tcctctacac ctgaagacaa aggcaaagtc aaatggggac cagaataaat cttagacccc |
| 11161 | acagtccttc ccatttccag ccctaatcta cagacaggaa tgcccttcag gtttcttccc |
| 11221 | tcccccctct tgacctaccc cagatatttg tgtggaagag gaggaatcac catttacaag |
| 11281 | gtggacaaat gctaatattt ttatctagaa agaagagtga gtgttaactt ttattttttt |
| 11341 | ccttctgggg ggtctgttga ctcctttctt ttgggtgctg cctataaatc ttggaggaat |
| 11401 | catttctcct cctcaaaaac tgattcagaa actgacttgg ggaaggaatt taatactttg |
| 11461 | aagtcatgag atgcaccatc gaggctaccc ccaagaagaa gcagaagaga agttggtaat |
| 11521 | gagaggggat tagaggtcct cccttcagta gggctgtgaa aacctcatca ctggaggtaa |
| 11581 | aagcacaagc aatgcctgtg gacaagatgt cattcattca ctcagcaaat gttcatggat |
| 11641 | caccggctac caaggtacca ggcaccatgc taggtattgg ggaagagaga ctgaagtcac |
| 11701 | aacccctgac tgctcctcaa aagctaacgg ttgcacctcc aagtggctgg gtctgttctt |
| 11761 | actcttggag ggaattctga gaagacagca cagaattgta aaccttccct tttgaccctt |
| 11821 | ttggatttta tcaggtgtaa acaaaaagct gaacagttac ttcaaagata tgtgtgtata |
| 11881 | ttcagttttt tattgttaag ctgatatttt aaagatttct gagctagcag gcatgtggga |
| 11941 | aggaaggctc tgtcttcaac tctttgaccc tccatgtgta ccatagaggg gggaaaggtg |
| 12001 | gtattttcac tttgatgagg ttggtaaatg tttttagatc ttctggtaag cattatgttt |
| 12061 | gttaatacat atttattaga gtgatgtttt aagttaataa agtattaaga gtatta |

Neurotrophin 3 (NTF3)

The NTF3 gene encodes the neurotrophin 3 protein, and has homology to sulfate transporters. NTF3 is expressed in inner hair cells and in surrounding supporting cells in the adult cochlea. NTF3 supports connectivity to spiral ganglia-like neurons (SGN). NTF3 induces synapse regeneration and SGN protection after damage (Wan et al. (2014) Elife 3; Budenz et al. (2015) Sci Rep 5:8619; Suzuki et al. (2016) Sci Rep 6:24907).

The human NTF3 gene is located on chromosome 12p13. It contains 2 exons encompassing ˜63 kilobases (kb) (NCBI Accession No. NG_050629.1). The full-length wildtype NTF3 protein expressed from the human NTF3 gene is 270 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype NTF3 protein is or includes the sequence of SEQ ID NO: 26. Non-limiting examples of a nucleic acid encoding a wildtype NTF3 protein is or includes SEQ ID NO: 29. As can be appreciated in the art, at least some or all of the codons in SEQ ID NO: 29 can be codon-optimized to allow for optimal expression in a non-human primate.

Human Full-length Wildtype NTF3 Protein
(SEQ ID NO: 26)
MVTFATILQVNKVMSILFYVIFLAYLRGIQGNNMDQRSLPEDSLNSLIIK
LIQADILKNKLSKQMVDVKENYQSTLPKAEAPREPERGGPAKSAFQPVIA
MDTELLRQQRRYNSPRVLLSDSTPLEPPPLYLMEDYVGSPVVANRTSRRK
RYAEHKSHRGEYSVCDSESLWVTDKSSAIDIRGHQVTVLGEIKTGNSPVK
QYFYETRCKEARPVKNGCRGIDDKHWNSQCKTSQTYVRALTSENNKLVGW
RWIRIDTSCVCALSRKIGRT
Mouse Full-length Wildtype NTF3 Protein
(SEQ ID NO: 27)
MSILFYVIFLAYLRGIQGNSMDQRSLPEDSLNSLIIKLIQADILKNKLSK
QMVDVKENYQSTLPKAEAPREPEQGEATRSEFQPMIATDTELLRQQRRYN
SPRVLLSDSTPLEPPPLYLMEDYVGNPVVANRTSPRRKRYAEHKSHRGEY
SVCDSESLWVTDKSSAIDIRGHQVTVLGEIKTGNSPVKQYFYETRCKEAR
PVKNGCRGIDDKHWNSQCKTSQTYVRALTSENNKLVGWRWIRIDTSCVCA
LSRKIGRT
Rat Full-length Wildtype NTF3 Protein
(SEQ ID NO: 28)
MSILFYVIFLAYLRGIQGNNMDQRSLPEDSLNSLIIKLIQADILKNKLSK
QMVDVKENYQSTLPKAEAPREPEQGEATRSEFQPMIATDTELLRQQRRYN
SPRVLLSDSTPLEPPPLYLMEDYVGNPVVTNRTSPRRKRYAEHKSHRGEY
SVCDSESLWVTDKSSAIDIRGHQVTVLGEIKTGNSPVKQYFYETRCKEAR
PVKNGCRGIDDKHWNSQCKTSQTYVRALTSENNKLVGWRWIRIDTSCVCA
LSRKIGRT
Human Wildtype NTF3 cDNA
(SEQ ID NO: 29)
atggttacttttgccacgatcttacaggtgaacaaggtgatgtccatctt
gttttatgtgatatttctcgcttatctccgtggcatccaaggtaacaaca
tggatcaaaggagtttgccagaagactcgctcaattccctcattattaag
ctgatccaggcagatattttgaaaaacaagctctccaagcagatggtgga
cgttaaggaaaattaccagagcaccctgcccaaagctgaggctccccgag
agccggagcggggagggcccgccaagtcagcattccagccggtgattgca
atggacaccgaactgctgcgacaacagagacgctacaactcaccgcgggt
cctgctgagcgacagcacccccttggagcccccgcccttgtatctcatgg
aggattacgtgggcagccccgtggtggcgaacagaacatcacggcggaaa
cggtacgcggagcataagagtcaccgaggggagtactcggtatgtgacag
tgagagtctgtgggtgaccgacaagtcatcggccatcgacattcggggac
accaggtcacggtgctgggggagatcaaaacgggcaactctcccgtcaaa
caatatttttatgaaacgcgatgtaaggaagccaggccggtcaaaaacgg
ttgcaggggtattgatgataaacactggaactctcagtgcaaaacatccc
aaacctacgtccgagcactgacttcagagaacaataaactcgtgggctgg
cggtggatacggatagacacgtcctgtgtgtgtgccttgtcgagaaaaat
cggaagaacatga

A non-limiting example of a human wildtype NTF3 genomic DNA sequence is SEQ ID NO: 30. The exons in SEQ ID NO: 30 are: nucleotide positions 1-229 (exon 1) and nucleotide positions 62081-63186 (exon 2). The intron in SEQ ID NO: 30 is nucleotide positions 230-62080 (intron 1).

| Human Wildtype NTF3 Gene | |
|---|---|
| (SEQ ID NO: 30) | |
| 1 | agttgaagct cctctccctt ccgaacagct ccgcgcaccg ccccgcgacg cagcccggcg |
| 61 | caactacttt cttctctctc ctttctttct tcctctcctt tttcccctgc tgggtagtgg |
| 121 | ctgcggcggg gtgggggaga ctttgaatga ccgagctcgc gtccaccttt ctcttcatgt |
| 181 | cgacgtccct ggaaacggcc acacggatgc catggttact tttgccacgg taaggggagg |
| 241 | cggcgggcac cttgggtggg caggtttggg gatgggggtc cacgtgggga gggattttcc |
| 301 | agtggactgg tgcggggggc cccagatccg catcccgccc cacccccatc gcgccgcgct |
| 361 | cactcacttt cccgggcttg tgtcttcccc aaagtttgcg ctgggatctg ctcaggccga |
| 421 | agcgcaaccg cagccacccc gctacacaca cacacacaca cacacacaca cacacacaca |
| 481 | cacacacaca cacagacacg gacacccttc tccacctcct cccctcttgt ccctcggctg |
| 541 | cccaagaagc ttccctcaat ctgggaaagt gatcaggttt aagggacctg gattggaaag |
| 601 | ggtgggggca gaagagggga aatggggacg acgaaagagc aggaaagaga ttcaacagaa |
| 661 | tcaaccaccc accactccca accgacccgc ctgctcctcc gagaaagctc ctagcgcatc |
| 721 | ctataacaaa agggggtggc agacagaact ccgggcgggg aggtgccgcg gcagctcccc |
| 781 | tgcacacgcc ctgcactctg ccggccgctg agcctgattc tcagctcgcc ccagcaccac |
| 841 | tctggcccgg gcgtgggctg gggggagggg acgcgcagct caggacccgg aacctcgcgt |
| 901 | tccagttttg ggagttggga ctcactgcca cgcgccgcgt acctgcgttg gagttccccg |
| 961 | aaagggtttt ttcagaaaag acctcgcgcc ccgggctcct cttggccagc gcccacccgg |
| 1021 | tggccacccc accctgggcc tttgcgcaga tgttggagct ccgtacgcag cccgcacatc |
| 1081 | tgggacccct ccggggagcg gcgggcaccc gggcccggcc atcccagggg atctccttgc |
| 1141 | ggtatcgtcc agcctgttct cggactttga gcggtggcgt gggaggccgg gagacctggg |
| 1201 | cacccgcgca gccagccagg tcggagttta aaggtcccac gacggaccga actgtcccat |
| 1261 | tgccccagag ctttactcag tggtggatgc tcctgatgaa atttgggacg cttgggagtt |
| 1321 | gaaggttagg gacaggaggg gcgagggccg agggcatggg atgggggagt aggattctgc |
| 1381 | ttgttgctct ccgcgggagt gggtgcgcgt ccaggaggcg ctgcttcttt gcgggagttt |
| 1441 | ggctgctgcg ttcattcgtc gtctgcgctt cagatgcacg gcactgagac ccttgcgtcc |
| 1501 | gacggtgtcg gggctgtgga ctagaaagga tcccttttgc tggaatcgag gctggggtgg |
| 1561 | gattgccggt gggggaaaca ccgaaaagat cgtctggcct cggcctctgg cggcgggcgg |
| 1621 | caggttctga gtccgaatgg aggttgctcc cgggagcgcc gggctcagag ctagagagct |
| 1681 | cgggagactg tgcgcctgtg gacttgttta tgtgtgtgaa gaggcggggg cgagggcctg |
| 1741 | ctgagagggg aggggagcct ggaaggggtg ggtgtttctc ctggagcctg atgtttgtaa |
| 1801 | ctcagctgat tatggagtgc actgagcgac ctgcttttta aataaagagg tgcccgctcc |
| 1861 | taccccgcaa aacagcgaac gaggagaaca tggaagcgct ctgtcctaaa cgtcaggatg |
| 1921 | ggagaaagtt gtaacataga ggagactttt ccagaggtcc tgttttcaca acactcagaa |
| 1981 | gttctccagc gtactcagcc tgcctcccgc ctgccctcaa ttcctttttg acatgtcaca |
| 2041 | caaagaaagc tgaaaggtag aatgtggagg ataaactcca accccctctg ccttgggcgc |
| 2101 | aaacacacag acttaggtat cgtgttagga ggtaaggggg ttggaaaata atgcaggctc |
| 2161 | cggtagacag tgttgaaggg agatagaaag tctggggtat ttcccctagg gagagtgtgt |
| 2221 | gggattttgt gttggtgagg actggagtgt agctggactt agagggtttg gttgtgtgtg |
| 2281 | aatgggatat gcttatctat ggagtgagat tgtgaccatt gagtgagtgg tcagggaggg |
| 2341 | atggggatgt ttttccaaag tgtgtgtgtg tgtgtgtgtg tgtgtgtgtg tgtaggggaa |
| 2401 | ggtggcaaaa gagctggagt cctatccctc tttggtgcct gggaggtgtg tgtttctggg |
| 2461 | gagtctgtcg gatgggcatc tgtgtgctta gcgggagaca agagcttgca ggaacatagg |
| 2521 | tgtagctaat gcgaaatctg tgcctatgaa gggtgtcaga gcaggaggag aggtgcagcc |
| 2581 | tgttgaagac agatggcttg tgcatgcctg cacaaacaca tggagtgggt gtggtgtagg |
| 2641 | agggcggctg tgtgtgtgag ggaaaggtgg atgcatgtgg aaggttgggc gcctgaggat |
| 2701 | taccaaaggg atgtgtgtgg ggggtggggg aagattatct gaggagcacg gggtttgaac |
| 2761 | ttcatgaggg tgcactggga aatttggctg gaatggcact ggggcatgta gggggtagaa |
| 2821 | aagtgtgagc tcagatcctc tttatggaaa gacaaagcat ttgcagggaa ggacaccggg |
| 2881 | ttggtgttgg tgtttgcctc ccccctcctt cctccccaga gagaagagaa ttggaggagg |
| 2941 | ettagaatga gggatgtaca tttaagggga aaagtccttg tgcattggca ctgggtggga |
| 3001 | acaggaagag atgtgtgcat aagactgaag taagtagagg aatgcactat ctgtgcctgg |
| 3061 | tgtcagggat gcatgttgta taatgttttg taatgggttt cttatgtaat aggagaattc |
| 3121 | actatcacca gttcatattg ataatcaatt aaagctaatg gagtgcctac tggatgctca |
| 3181 | gcccattgct gggtactggg gcatggcaca ctgccctgga aaaattcctt ctccaggctc |
| 3241 | agtaaaactg atgcaatttt gccagaaata gacagagctc gacgcttgtt gaggtttctt |
| 3301 | caaggtctat aattcagttt ctagagatgt ttatggatct tgggggccaa agggaagctg |
| 3361 | acttgaaata tatctctggt tttgtaaaaa gtggggcata aagagaggga tgatgtagac |
| 3421 | ttttgtgtat tgggaaggga gctggacggt gttgttgttt cttgtgtggg gtgtgggctg |
| 3481 | ggaattggag ggtgctgcac ttcaggtcag aatgcaggtc cagatccctc taaggattga |
| 3541 | gtttctaaga gtacacagag cctccttcat agctggaatg agacctgtta tgtggagctg |
| 3601 | gagaatggtg aagtcatgat caaggaagag atggactggg agtgtttgtt catctttgat |
| 3661 | gttttctggg ctacgagtgt aaagctaacc tgagtgtgtg ttgggggggt gggtatgcat |
| 3721 | gcaggtgcgt gcacatgcac tttggcctgg agtaacgtaa ctgggagaaa actagcgagg |
| 3781 | tggataggag gggtggatat tctggctctt gtgaaggggg tgccagtatg tgtttttgca |
| 3841 | ggaattctgt gtgtctgggg aaggataaaa attgtcttgg atactcagat gtgcatcttt |
| 3901 | agcgaaaaaa ggtgtacaca aaagaagggc cgtagacttt ggggaatgag acaaaggtgt |
| 3961 | cgttgatcgg gtccataccc tgagatggct ggtgaggcaa ctgggtagga acagagtgaa |
| 4021 | ggctgtgctt ttgtgatgca cacatctgcc tgtctggagc agacgatggg gatccccatt |
| 4081 | tgattaggtt ctgaccaccg ttgggcatat gtttgggagg actttatttg actcatgaca |
| 4141 | tttttgaatt catgaagtta cagctgtgcc attttaaggg tctaccttat ttttgagagg |
| 4201 | gaaactcatc tgtgtaagag atagtttata tagggcttga ctattggtca gaagatccgg |
| 4261 | ggcagatatt tctgagagca gggtggtgaa gtgcaagact ggattgggaa tgtcttagtt |
| 4321 | ctagtccagt tctaccactt gggaaaagtg tgactttggg gacattttta atctctataa |
| 4381 | ctcttctgtt tctctttttg taaaggggca atataatgac tcaaggagaa ggaaagttca |
| 4441 | ctcctctttt cccccatcaa aacttgatgt gtttagttgt atacagccat acaaagatga |
| 4501 | aatgtaatta aatacaggtg ctagcacgta tgtaggtcat tgtgaaaata ggatctggac |
| 4561 | ccctttctct ttacaatgca acttaatcag taatcatgat gcccacagca tctctactgc |
| 4621 | tcactcattg tgtaaataaa actttttata actgcctcag tttactcatc cagaagctgg |
| 4681 | aggcagccaa acctagatta tagatgatgg aaatctttaa gatctttgag ctattcagag |
| 4741 | gaaaggtagt ctaagaatca atgttattaa tgatcagaga cagacatttc tttatttttt |
| 4801 | aacatcagct atgcagacca cagcaccaaa agaaatgtga cgtggagaac agcaaaaata |
| 4861 | ataattgccc ttgacatagg caccatttga ataaaagaag agcaaagtct agcctgcctg |
| 4921 | gtaagaggaa agcctgggct ttgggtcttt gaaattcttt gtgcccagtg cagcaagttg |
| 4981 | ggtacagcca ctttccccag agatgaagat ggagtcgtag aggggcatgg aaacacactg |
| 5041 | aaacctccga ttcctcccag aagtcagtac atgctgtcaa ctcctggctc agagtcaggc |
| 5101 | tcccatcctt gagttcagtg gccataaata gtcaggagct aagctatttc caagaggcac |
| 5161 | taaactaatt ctgttgcttt tcttggcgag attgggccaa ttaagttgat tggaagaggt |
| 5221 | cactgctgat gggtctcaca ttccccaggc gggacaggcc agcagagttt aattacataa |
| 5281 | catttcccat gtcttcactt ccactcccag atgtatcaga tgttcaggcc tctcctcttt |
| 5341 | ccctacattc aggatgcctg acggagcagg cggcttctgc tacaagcttc tccagacctt |
| 5401 | ttcctgagca gttacagcac tcccacccca actggaggga atgcaagtct gtgcattgcc |
| 5461 | tggaagtgag agaggggatg caggctgaca cagaagatct ttagagccca ccaccctgcc |
| 5521 | tcaagcctca cccgcggctg ttttactctt taacgaggca ggcagtttct ggcgttgggg |
| 5581 | atgttgaact aatgaccgtt gctagggttt tagatttagt tttatttaaa aatgagtttg |
| 5641 | ttggaaaagg accaaatctt ctactggaga caagcctaag ccccgtgtgg tgctgctgat |
| 5701 | aaggtctgtg gatgtacttt gtgcatggac aggcatgtgt ggagtaagag gagagcacag |
| 5761 | gatgtgcgtg cgtgcgtgct gggggacacg tgagcgtagg agatgccctg ggtagatggg |
| 5821 | gcatttgtag agcttatgtt atgggtggca ggtatctgga tgcttggctc agattggatt |
| 5881 | gtcattggat tgcttcttac aggtgggaca gcctgagaaa aaagttgggg acaccttcct |
| 5941 | tcagatcatt tttatatggg catgttgaac cttgaagtat gaggccagtg tagatattac |
| 6001 | ctcattcttg cctggatgga tttctaacaa gattgaggct ggacaaaaaa aaaaaaagtc |
| 6061 | tttcagagga ctcattttag aacctgtgag aacacatgta gatggcccca gagcgttaaa |
| 6121 | gtattttttg ttgttgttaa tagttttcca taactcagtc ttttgtttta cctaaaaata |
| 6181 | ctgagaggct agaggttgca cacaccccca catggtcaga atgcatttgc tgttcatgtc |
| 6241 | cccatctgtg ccatggtcct tctcaaatag gattaattgg gaactacgga gagcttgccg |
| 6301 | cagctttggt tgtcctcaca ggacaaacct atgactgacc gtgatgtcat tgaaacagaa |
| 6361 | cagattttga cgtgaaattt cgtgtggtct gttgtgattg ctcacttttt agtaacatct |
| 6421 | ccagagaaga catttgttga gccccatttt tttttgtggg tttgttcagt cagagatttg |
| 6481 | ttggaagatt taaggttctc caacattcaa ggccagagat tttggaggaa acagtagcta |
| 6541 | acaagagccc aggccagggt gaccttcagt agcctgaggc agcaggtagc tgcctgactt |
| 6601 | agaagtgaag gaccaggttc agtgactggg ctctgtcact aattagctgc tactgtgtga |
| 6661 | ccctggacat tctctgtagg acatgacttt gccatctgta aatgaagggc ttgaattaga |
| 6721 | agatctccca gtcccattcc agccctgtca ctgtaggatt ccacagtagc cacatcctcc |
| 6781 | tacctaatca gtgctaatta cctcacttgg agtgcaccct ggcacgccct taacgagctc |
| 6841 | ctgtttttat ctttggggaa tggctgagtc aaaggcagaa aagcgtgcaa aaaatttgcc |
| 6901 | agagtgtttc cttttggcca agaatgctct aaacattggc ttagcctaca cagggtgtgc |
| 6961 | agagctgaaa ggggtgggat ttggctttct ggtaggtctg atgctaatca ttcagcttcc |
| 7021 | acgtgaatgt ctttcagtgg caatgtctct gactaaggta agcatccatc tttaagatga |
| 7081 | tggatgatga tgatgatgac agttaatatt agagtactta ctgggtgcca ggcattttta |
| 7141 | cagctattac atggcattat ctcagtactc aaaataatct catttacaga tgggggtact |
| 7201 | gaggttatgt gaggttaagg ggtatgctga tgttcttggc tttccagcct gaagtctgtg |
| 7261 | gcatcccaag cccatcacct gctagtcaga ttctgttttt tatgtctgtg atgtcatcca |
| 7321 | gatgggagct gggtccatct tcttctgcaa agttggtcac cccatagggt aacctgctcc |
| 7381 | ctgcccccgg aagcctcctt gtcatgtgca agacaaatgt ttaggttttc tagccacctg |
| 7441 | ctggcaagca tgtgtcagat ctgtctcagc ctccattggc cctctccctt tacttcttgt |
| 7501 | tctgttgaca agggaacctg cccttacaga atacggatat tgatttaagc cacaattgcc |
| 7561 | tccttgtaaa ctctataaaa ttggagctgg acatctggtt ttgatccaca cgcccacgag |
| 7621 | aggtggtagg tcccagcatt cttgagtcct ggaattgcac agaatcaaag tgaaaaagaa |
| 7681 | tatgaatgga gagcatgagg tatctttaga atttggaaga tggctttctt tgataggcta |
| 7741 | gggaactcac accatttaga ctggggccaa agttaaggta ggtttcttgc tgggaggttg |
| 7801 | gggggcaggg gtcactgcta aagactgagg tccccaggct gtgtttgatg cagagcagag |
| 7861 | cagctgtaga gccctccagc tgaggatttg ccactgcaca ccattctctg ggcagtacca |
| 7921 | gaaatagaga agccaaacag gttcttgggg tggatgttgc cctgtttgaa gattgtgact |
| 7981 | aaaggctcat tccttcttcc tccactgatt tcctcaccat cactaccaca tatacatcat |
| 8041 | caagacagtt aaggacaggt gactttgttc tgttgcttaa acatatttgc atgtacaaat |
| 8101 | aaatgttgaa cactaaccta atgcctatta gaccataggt actcaaaaat tatgtattta |
| 8161 | ctttgcttaa aataaccttt catgtgtagg gtgataattt ttttcttcat atggagtata |
| 8221 | tagcctttta aaaactgata gccaggttct tgcaatttac ttcccccttc cctctgtttc |
| 8281 | tagatctaag accccagcat ttaaaaatat ccttctctgg ctctaagtga tccctcaggg |
| 8341 | agtttgtttc aagttctcaa tctgagatta gtttcatccc acatctagag gacctgcttc |
| 8401 | taatggttca taaggtactt ccactgggtt caagaggaag acacagctct agaaatcacc |
| 8461 | tgctttccca acaggtgtgt cccatggtgg ggaagaagcc cagtcctcct aacacccacc |
| 8521 | tggctacctt aaatagcttg ccacagtctc cctgcagaga gcccagacta gctgtgtgac |
| 8581 | tttaagcaaa tcactttccc tccgtgggcc tcttttcctg gaaaagggag gagttggact |
| 8641 | cactagaggg tcacaaggac cctaccagtg tgacagatca catcttccat gactccgctg |
| 8701 | ctccctcggc cccacactgt ggcccatgcc tgggattttc aggtaaccct gctggcctag |
| 8761 | aggacttaca tagagtgatt tggagggggt ggaggaggaa attaagttat gtgatgccag |
| 8821 | ataagtcatt gaacatacgg atgagtgcat gtttatgaag cactgaagaa acaagcttgc |
| 8881 | cagattcgtg gcggatcttg gattggaagt tgggagtatg cgagaggttt atgtgggatt |
| 8941 | aaagttgtct gtgggtagct gaattcaata agctaaatca gagagatccc aaactctgca |
| 9001 | gagagatcag accccgcctc taactccaac agaggctaat tacaattttg ggaagtcctc |
| 9061 | gaaccacact aggtctcact tcctcatcta ttaagagcta ctgactttga ttatctctaa |
| 9121 | ggtctttctt gacttatcat taattaaatt gtgagaattg ggagctagta gaaaaatctg |
| 9181 | gacaaatgcg tggatggaga aggggatacc tcaaatggcc taaatataga gggaaatgtt |
| 9241 | ctgtacaagt gatacgttta ttcttgaatc ggagatagca ttggaggata tattctgtac |
| 9301 | aaatattgtg cttcaaatgc ttacccctcc atcatcatca atgatattaa aatcattgct |
| 9361 | taacctggga tatttgctta acctgggaag cctccctgca tttccccaga gagcctaagg |
| 9421 | ctcccttccc agcctcctga tgccacttag cagcctgtgt ttaatttgca ttcctagaat |
| 9481 | gtgacttcac agagaaaggg atctgttgtt ttcatttttg tgtcccccac cttgaatagc |
| 9541 | gacagccaca ggaatgtgtt gagtgaatga atgcgtgacc agccattgtg agctcagaat |
| 9601 | gtgcagcagc gatttgacag tgccaggaac ttggctcaga gaggcagtgc accttgccag |
| 9661 | ggcttcaaaa cagtgttacc tttatccccc ttttggcctg tcccaaggct aaggtcatga |
| 9721 | atgctcagcg cttggtggcc agaaaaggct gttcttactc ccttttagag acctttcttc |
| 9781 | cccacatctt agcctgctta gttctcctgc aaattggggc tcttggtcac agggaatcgg |
| 9841 | gtgtcattgc atacgcatta ggataactcc tcgttagaca ctcggaaagc aactcaaaac |
| 9901 | acacgtgtac agattattca ttaagcagtc tgttggtggt cacgtgctaa atgttgggga |
| 9961 | agatgcagag atgaatgtag tgctgctggc cttggggtct ggaaactggt gaaggaggca |
| 10021 | gtcaggttac aatcatagtg ctccagggag aacaagggaa gcaaggaaga tggaatacag |
| 10081 | ggttcccact tcaggtgttg ttttttagta caaaccatga ttgagaatcc tcttggttca |
| 10141 | ggagcaagct ggagggtcca ggaatggtct ccgcttccac atgacagtgg ttttcttgtc |
| 10201 | cctgaattcc cagggctagg gtgatggatg ctcagagacc attgtgatgg gagcccttgg |
| 10261 | tccttgtggg gagagtcggg ggaacacgca tgttgtacag gtgttggggc ttggggaggt |
| 10321 | gtaggtgtgc atgctcacca gggtacttct cagaagtatc cctgctcgtg gcatggtggg |
| 10381 | ggtgagtcca cctcaggccc ctaagctgtg cttctacttc ctttctaatt atacagcatg |
| 10441 | tattgtctct gtgaccactt ctgacacagg aacagactgc catctactgt ccacttctgt |
| 10501 | cctgagtccc tcctgctcaa ggaggcattt attttttctc atcctgtttt taatagatcc |
| 10561 | ggacaccagg agaaacagac gtggagtcct atttagtaat ttttttagaa tttagaaggg |
| 10621 | agccttcaaa aaacatagca catctgtcag atggtttccg tatctgttta caaagtatta |
| 10681 | ttgtaagggg ccactctgct gccactgagt cttgctagcc gctgtgcgag gtgccgtgtg |
| 10741 | ggataccaag agatacaaag caaggcctta ccttcaacag ccttgcagtc tacttgcgaa |
| 10801 | tacagtgtag atttgcagag aagcagacta caggatgtgg cagtagatat ttggtaagag |
| 10861 | gcatctgggt ggtagggact acagaggttt tgggagtttg gaggaaggag gagcccttat |
| 10921 | aggctggggg atttgggaaa gaccaatatg ggtctccttt tagcaatccc agtgtttagg |
| 10981 | aagaggcttc taagagctgc gcgatgttgt gggacaggct atgaactgga aactcaggaa |
| 11041 | cctggagggc ctgaggtcta gtccagcttc tggcagtcct gccctgtgac ccagggcctt |
| 11101 | gggttccgca tcctcaaagc aaggctgttg gtcttcttgt catgtaaggg cccccccatc |
| 11161 | cccgaccctg catcatgtac cactcttgga tgcagtggga ggtggtttcc tggtttccag |
| 11221 | cttgtcaaga gcaatagagt caattggatc ccatgcagga aggatacctg gatgcagggc |
| 11281 | cctgtgctga tgaccccaca tgcagcagga gagagagaat ttccccaggg gagttttggg |
| 11341 | tgctgttaaa atacaaaggg gggaaatgtt ccatgcaacc tctgcatgcc actcctctcc |
| 11401 | agagctttcc tggactttcc ttccaaaaat atatgtgcat ggtgctttgc gtccaagata |
| 11461 | gctaccccaa aagaaattgt attagtattc tagcattgtt catagctcaa atctaagatt |
| 11521 | ccttcacatt agaattcatt ccatgattgt ttttatggag gttttctttg agtatttgag |
| 11581 | acttttggta gctgtggatt aaaatgagga aaagattgtt ttctatagct cttttactcc |
| 11641 | ttgtctcttt ttctctgtct ttcttagcat ttaaaccacc accaaggaaa cctccagggt |
| 11701 | gtttattgca tattcttgcc ttttgatgtg tgtgtgtgca tgagagagag agaaaaaaaa |
| 11761 | agaaagggag agaggagagg cgagagagag tacaagagag aaagatattt tagactgtgg |
| 11821 | tctattagca tgtctaggaa atcaagttga atttggattg ggttacaggt tgagagacct |
| 11881 | cagctcgtca ctgtaatcct ctgacatttc ccttttactt tttggctctt gtctgaatca |
| 11941 | agaatacatc ttcccgttcc ctcttccatg tttacatctt cttttggggc agctgtataa |
| 12001 | agttactgtc tgtctacagc aagtctcctt aattcttttg gggactgctc gacagaggca |
| 12061 | cagtcaagga tagaaccatt agagacggtc agcttttgga caaagacaaa aaaatatgtt |
| 12121 | gaggaatagg agaaatgttc caattgtcga ttatgtgttt tcctgtggta tgctgatgtc |
| 12181 | tgtgactgtt gtccacagaa agagagcgtg agcaggggct ggggcagaga taaaagggag |
| 12241 | attctcccca aaccaacaaa tgtggagaga gagcaggagt ccccagcaac agatatggag |
| 12301 | ccaaacaacc ctgggaagta actaggaaac ttcctggtga cctgagatgt gttttgtaat |
| 12361 | cactggtgaa agtaggagga gatggggtag aggagagtta caggtagaga agttccgtgg |
| 12421 | gaggagaccc caaggtgaga gaaagcaggg acttaaagaa ctgaaagaag cccagggagg |
| 12481 | ggatgaacag agaacatccc atatatccca tatccagtat ggtggagagg tgggcgggag |
| 12541 | ccaactggga gcttactttt tttttcccag gggcatggga actatggaaa actctgaggg |
| 12601 | cacaattaag cttttgtttt taaaaagatc cctccggctg cagtgtggct ctgaataaag |
| 12661 | agttgttcaa tgaagaaagg aatggctgat ggttggattg atggaagaat taatgaatga |
| 12721 | gcaaacgaat gcatgagatg cagagggaac gcagcaagcc tgatttgatc tctggttact |
| 12781 | ttgaccagta ctggctccag gggcgattat ctcagcctgg gaggccaggg agtaatgcat |
| 12841 | tgattagaat gtctctggac acatggatta aaatatctga tattttaggg tggttgatag |
| 12901 | tggggaggac ttctgaaact ttttccctct tctatgcatt tccatcctaa tttgatttca |
| 12961 | ttcaggatca aaaaagaaga tggcttttga aattacatcc cagaaaatct gaaactgtgg |
| 13021 | cattgacttg ctccagagag ggctgcttgc atggaagacc ttttcatagg ctcatcgtgg |
| 13081 | aatagggaca gatgataaag tttcttgggc atatgaaggg gtcccagatt tctggacgtc |
| 13141 | agatccaccc atagatgatt ccttggatta aatgatgtgt gtgtgtatgt gtgtgtgtgt |
| 13201 | gtgtgtgtgt gtgtgtgtgt gtgtttaaag ttttaaagtc tcctgaaaat taccaagtgc |
| 13261 | tactgaacat tttttttgtc agtagtggat atctggataa tttctttcaa ggccacattg |
| 13321 | cttagcatgt ataaggaaag tgtgtgcggc agagacccag atggacagcc ggcatgccat |
| 13381 | ccagttgctt ggggagtgtg aatactcctt ggcaaagcca aagggagcca aagaggacct |
| 13441 | ctagtgtctt tgctctccca tgtcctgact attccaatct cactttgcat tttgagatct |
| 13501 | tttaactttt gctaacagtg attgctcagg atgtcattca ggccaattaa atttgaatgt |
| 13561 | ctagggttgg gacagaagct cacgaggtga atgcaatgtg cagccacgtt gagaaccact |
| 13621 | agctgacact gctaatctac tctgctctcc tccctgcctt ttggcggctt tgccatgata |
| 13681 | tctgtcccat cctcacctca gtttgctgag aactcctcac ttagttaagg aagttcttct |
| 13741 | aaggatgttc aactaatatg ggctaaggcc tcctatcccc taaaaatcca gcatttgcct |
| 13801 | gagaaattgg acgctaggag gataagacag gttcttagca gattctgaag cactcaccgt |
| 13861 | ctctcatctg ctgaagggtg tattgaggat gaatgatggg gaacagcagg gagaaagacc |
| 13921 | agcatccata gggcagtaac agggtgcaga cacctattta tatgtgtcat cgtagttccc |
| 13981 | ctgacagctc ccagaaccag atattatacc aggcagatat accaggctta caggcaccta |
| 14041 | tttatatgtg tcatctcctt agttccccca acaactcaca gaggcagata ttataccagg |
| 14101 | cttgagggca caacaactca cagaggcaga tattatacca gacttgaggg cacagtttgc |
| 14161 | ataatgcatt cataatgcaa agtggtttgt gatcttggaa tgatcagact tcctacactg |
| 14221 | gttctcccca gtaatttgca tatcagccag ataccctcct ctagggttag ctatgatcag |
| 14281 | ggctgctgat ttctggcact gatggctcaa tgggaaggaa agcgcctatc ccctctgttc |
| 14341 | tgtgggattc agcaatcttg ggctggtcct tccaggaggc ccaacctgag gacatgcttt |
| 14401 | aaccaacagg cctttatatt gagaaaatag tagttctctt ccttgccatt tccctgttag |
| 14461 | gaagccatgg cttgcaggac agccagggag tagaggttca gagaaggagg ctaaaaaaat |
| 14521 | caagacctga aaaagttaag tttccaacca tatattccca aattgtagag tggaccatat |
| 14581 | ggaatcattg caatccctgg gtttctcatt tacaactttt gcaaaagata ttactatgta |
| 14641 | tgctgtcagg catgctggag ccggatgtct agcacccatt gctaagtgca tgcttcggta |
| 14701 | agagctttgg acttgggatt ctccagactg ctgagtgacc tacgtgtagt ttagcaacat |
| 14761 | aatgcttgga atgagccacg ccgacctgat gcgagaaccg gatggtagcc gaagagagca |
| 14821 | ggccaggtag tgagacaact atgatctgca gaggtgggca gcggggtgga gacaggcttc |
| 14881 | ttctgcacca agtactgcta cctgaactgt acatcacaga gacaagccct gccgagagca |
| 14941 | gtggaagcag gaaaacacag gcctatctct gtgtctgtca tgtaagtaag tctctcttgg |
| 15001 | ctgtgtgtac ctgccaccat caccccatcc caccccaaat atacagcatc accttcagct |
| 15061 | atagcttttg gtttttgatt aaaggacagt aatatttcca ggagggagaa gagtgatgaa |
| 15121 | cagaagcatt tatgtatgga cactgggcaa tttattgttt ttcaaagtct aatctagaac |
| 15181 | atgtttgcaa gaaagcgtga attgagtaaa aagtttacta tcgtattaag gactgttagg |
| 15241 | tacaatgact gaaggaggag ttaagagtga tccttcaata attcccctgt gggattatgc |
| 15301 | acatttaagg aaaaatgttc ttcaggctat ttctgcctta gagctaggca tcattacagc |
| 15361 | gaaatagaga actaaccttt aagcaagagg aaccaagttc tagttctatt tctgtcaatc |
| 15421 | actgtacgca caacctctca aagccttagt ttcctctttg caaagtggga taataaaccc |
| 15481 | tacctactta acacaatgtg gggtttcagg tgagatgatg cataggatcg tgcttggcaa |
| 15541 | gctgtaaatc tgtaaattac aaatatatat tatggtttca actggtacat tcctaagcga |
| 15601 | atagcacatt gctctgttgg gaagacggct cttctccaag tcaggctggg ataatgttcc |
| 15661 | ctgacaagac actgccatac ctaggtgttc cccaaacatt gtctctggga accttgagga |
| 15721 | agcaccataa gacatgggaa gaaatgttac agcgctggct tgaaagaata acaatgtatc |
| 15781 | agtctactta tttctgataa tgtcatcttg ggataagaga ctcagggtag cttagtgagg |
| 15841 | gacatgggca tgcactgcac agtaaaaatg gtgtccagga aacctgggtt tatttcagta |
| 15901 | tgggttgccc acacttctgc caacccagtc ccctacttcg tccccagctg ctcttgatga |
| 15961 | actctctgca cacacttgca cctgtatctg tgaaacagag ctcctcctct tacatgagaa |
| 16021 | tggatctggt tgcaaatcta atagattccg ctaccacaat gtcccctgcc ttttttgttt |
| 16081 | acttcattta tgaaaatacc cttgaaacat ccatagtccc attttgtaga catggtgctt |
| 16141 | tatgtctttg agattattaa atactcatgc tcctttctga ttgctgtttt cacctcttct |
| 16201 | ttaggcttgg gcttttctat tggtggaatt tgctgttcct tttcatggtg ctggctttcc |
| 16261 | tgagttgggt ggttgctaat tcttattgcc agtttgtctt ctgtgacaga ttctccgaca |
| 16321 | tgcctcggat gtggcttccg tgcttggctt tagcttttta tctgggctct cgtgtcctga |
| 16381 | atatttacct tctttcagga atttctcaca gcttctagcc aatgaaagtc ccccttctta |
| 16441 | ttgtcaaacc cagacagtta taattttatt ttaaatgtac tttttgttac attgttttgg |
| 16501 | gagtagggct ggaaggactt gctaatggat gagtggtgga aagtgagaga aagaaagata |
| 16561 | tgatggaaga aggagcaatc aaagccccag aaggaagcta tcgcattgtt cttggattcc |
| 16621 | taagcctgcc agaaagagct gacttacttt acagtttctg agagaactat gtgtgctatt |
| 16681 | actagaagca caagagaaag aaaaatagga tgttcagcat gcttcattta tctaatgtga |
| 16741 | aaaatgaact ctgcccagtg acttaatggg cataaactct gtttctaaaa aagccactca |
| 16801 | ttcggcaacg cacttctgag ttcttgatat ggtaaggtat tgtgttctgt gctggacagg |
| 16861 | aatacaaaaa tgcacggttc ctcacctcca agaacttata gtacatgtag ggaaataaga |
| 16921 | caacccctat tgaatatcac tcaagatgga aatgactagg gccatggaat gtaacacaca |
| 16981 | gagggtacct ggagttccta agacttctga tggatgattg aggagagccc tggatcaggt |
| 17041 | aagcaatttg aagggatagg gatagcacag acagcacagg ggtggaaaca agtgtggagt |
| 17101 | gtcgagagct tgaccagtac gcctgaaagg gagggagtgt acacagagcg ttaataggag |
| 17161 | ctctgtctcg aggcagcttc cctcagcccc tcccaggaca tcgaggtttt gggagaaaga |
| 17221 | gcctattgct cactctcacg gctcttctcc tttttctctg ctttcagttt gttctttgaa |
| 17281 | ctttttggaa acttcccctg ttctttcttt aacactgtgc ttcatctctt ggggttctac |
| 17341 | gttttgcagg ttgtagtgct tgagatccag ccttcccaaa tgatttctct gaatttagta |
| 17401 | tttggtatgg gttttgctat tttgctgcca tcccagccct agcaaagaaa cgacttaccc |
| 17461 | ggagtatgga cagggcttca gagaaaaccc ctaacattcc tgactcccga ctttacagag |
| 17521 | ctctgccaaa ccttgccttg cgggagtaag aaaagcgcta acaagccatc ctctttggtg |
| 17581 | tcaagtgcag acaaatcact tagcccctct gaggtcctcc aacagtaagc tactggtttg |
| 17641 | tgaaacccca ggataatcca tctgatttca gtcctgcatt tagtcactta gaacattctc |
| 17701 | gcacatgcat ttgctggctc atgtacatac gaatatacac atatccctct ttgtgccctt |
| 17761 | tcttagcctc tgatgatttc ttctcctcca ggaggcagga atccaaggct tataaaccat |
| 17821 | gacttctggg aagttttttc ttctgcttaa ccagggtatc attgtttttt ctgccttccc |
| 17881 | ctggagaatc actggccact gccctagtgg ttggggcaag gatcagaggt agcttgcatt |
| 17941 | ctggggtttg tccccaaagc ctcggtggga ctctgcattg gggtctgtag cctggatcca |
| 18001 | ctccagtacc ttaactaatc tcttgactcc cagatggtcc aaaatatgtg gatttagaag |
| 18061 | agcaacagac agctgttcct ctgggcctct ccaagaacac ggtttggtgt ctagaccacc |
| 18121 | ttagagaaac atggcagagg aaatcatggt ggagcagcat ggaaacaggt gaaacccaga |
| 18181 | cttagtacct tgttaaattc catcctggag tggagatacc agaggagcag atattacctt |
| 18241 | tattaactga tagaaatgtt tggggatttc tctgacttct tgtagggttg gataagcccc |
| 18301 | aaagtgaaga gaattttgct ccttgtttta gccattagga aactcaagac cctgctacag |
| 18361 | tgctattggt ttaatttttc cctatcacat tgcctctgca acttctgaat ggttgcagcc |
| 18421 | atttcttaaa atttccctgc attgtcactc agacaacaag aatagatttg gccttcttca |
| 18481 | tctcaaaata atggtcatga ttaatagtta ttggactggg aacagtgctc agccctctgt |
| 18541 | acgtgatctc aggaatcctc acagtactca atgaaatagc aattttatta tctcattttt |
| 18601 | gcagacaaag caacggaaac ttccacacat tttctacatt gcacctaaga tcatctgaga |
| 18661 | aactatgctg tacttgtttt tctaatgtat gatctgattt ttctattata atgttaattc |
| 18721 | tatgaggaca gggctttctg tggccttgct tcattgctgt atctccagca cctggactac |
| 18781 | tgcatggcac ctggtagtta cttagtaaag gtttttcaaa tgactgagta actcatccaa |
| 18841 | gattaaatgt ctaggaagtg gtggcaccaa gcttaggacg actcttttct gattccagag |
| 18901 | tccagacagc cctaaccact atcccacact accttcttgt ttatttttaa atcattttcc |
| 18961 | ttcccttcaa tccctctcca gtgccttaca ccttcttgct gtaatttgaa gcatggccac |
| 19021 | agtaagctac ctcaagtttc tcatctgtaa aatggggata atataatgaa ctaccttatg |
| 19081 | ggattgtacc cctctgcatg gtagcctcat cctactgtgc ctcctaacca cggcctttaa |
| 19141 | atcagcaggt atagttaata tatttagttc ttttaatcta atctgaaaca caaagcattt |
| 19201 | gcttccttaa ttcaagattt ttggctttgc ctagactaag cttaaaacca aagaagtact |
| 19261 | gcagaactga ctgaggctgc cagaagtacc acactcttgc acccagccag tgggaagtgg |
| 19321 | aaagataaca gctaagcctt tggggatcct tccagaagta gtgatgacgt acagcattct |
| 19381 | ttctgattat gaagtaaata tctgttctaa tgtatgttca acatagagag ttaagaaaat |
| 19441 | ggggaaagaa taaagagtaa aacaatgacc agaaatacct tcaataccct ttgacattct |
| 19501 | ttctctgtgt gtgcatgtgt ttgtgtgtct ttgtttctgt gtctgtatat gtgtatttcc |
| 19561 | tttatttttg tttttttact ttaatgtaat ttttagagac aaggtcttac tttgtcttgt |
| 19621 | agactcgcgt gtggtggcgt ggcactcatg gttcactgca acctccaatt cctgggctca |
| 19681 | agcgatcctc ttacctcagc ctcctgtgta gttaggactg caggcatgca tcaccattcc |
| 19741 | tggctaattt ttcaattttt gtgaagacgg gctctcacta tgttactcag gatggtctca |
| 19801 | aactcctggc ccgaagcaat cctatcacct tggcctccca aagtgctggg attacaggcg |
| 19861 | tgagcgacca tgcccagctc ccttttataa ataaggggct caccatacaa tataaccagt |
| 19921 | ttttacctgg cattttccag tcattattgc attgtacgta tctccccatg tctttttctt |
| 19981 | ttcttttttt tttttttttt gttgacggag tctcactctc ttgccgggct ggaaggcagt |
| 20041 | ggcgcaatct cagctcactg caacctccgc ctcccgggtt caagtgattc tcctgcctca |
| 20101 | gcctcccgag tagctgggac tacaggcgcc cgccaccacg cccagctaat ttttgtattt |
| 20161 | ttagtagaga cggggtttca ccatgtcagc ccaggaaggt ctcgatctct tgacctcgtg |
| 20221 | atccacccgc ctcggcttcc caaagtgctg gaattacagg cgtgagccac cgcgcctggc |
| 20281 | cctctccatg tctttaatta ttcttgcata agatgacttt tcactgcata atattccatc |
| 20341 | acataccact ctttaaccat tttgcttctg gggcacattt tccttttggt cacacttttt |
| 20401 | atactacagt tgccatcctt ttacatacat ttgaatacac atatctggct attctctcag |
| 20461 | aatagatttc cagacgttac ctttccaagc ttgaagatgt taacatttta aagaaagatg |
| 20521 | gttattcttg aaagccctga cagctctgag tggggagccg gggctgatgg ttaccacagg |
| 20581 | atagcggaaa ggcacactgg ctggcctgtg tgtactcacg catcccccca cctagggcag |
| 20641 | ccttgggaag agcactcagg attatgagaa agactgtcgc ctcccctttg cttcattagc |
| 20701 | tgatcctcta agcatatgtg ctttcttggt ctaattttcg gattggtctt ctcctatatt |
| 20761 | ctcttcctac tccccacccc gaccttacag ctaagtgcac atctcatgta gtgcagtggg |
| 20821 | aaagaaccgt aaggcagaag ccgggctgac ttggctgtga atcccagctc catcacttgc |
| 20881 | tggccaggtg actgagtaag atcgtttaca catccatcat cctcaagttt ctcatctgta |
| 20941 | aaatggggat aataatgtaa ctgccttatg gcattatata aggattgtat gactgaacac |
| 21001 | atgtagaatg cttagaacaa tgcctggcat atatgaagca tttaatacat ggtgtattaa |
| 21061 | attagttttg aaaagaataa attaataaca atgatgaaca tttttgatac ctattttcct |
| 21121 | attgttttga ctctcaaagc cagttgcaag catatttagc actgtgatgt atgtgtgact |
| 21181 | tactgcaaag tcttttttcc agtccctgat accagctctc tcttcacctt cagtgtttcc |
| 21241 | tacccctcct gcctcccctt ccctaagaat attgctgttt cacagagtgt aggctttcct |
| 21301 | ctggcttcca gatctgccca catatgcaca cttctctttc ccatccctgt tggactcttt |
| 21361 | ctccttatca gtttatttgt tccagttggg aagaactgga acctggtcgg cagcttttcc |
| 21421 | agttggcttt atctgtgcgc tgcattgtaa aactgttctc tcttgcttag aaatctcttt |
| 21481 | gatccatgtt tagctgtatt tattcttcca acagatgttt tgggtagtga gaggattttc |
| 21541 | ttctcgcatt tgcctagtct catgctcctt catgcttccc acttgttcgg gatctttttg |
| 21601 | ccagctgacc acagacaggg gccatctgtc gtgaaggtct ccctggccca gcagaccagg |
| 21661 | aatggcccag caaccaagac tttctgaagg gcttagtgaa ggggaggagg gaggaagatg |
| 21721 | ttggagaact gtgtagggta gagtttgagt ttcccagaca cattccagga gctcttttga |
| 21781 | tccaaggtat acatgatttg gcttgtgctc tgtggcaggt taacaaaaac acaaccttcc |
| 21841 | attgtctcct gtagacaaca gagtgaggcc cttgggcatg gcaggtagcc taagactacc |
| 21901 | cctgagagtt gggaagtgta tgagtctcct ggggctgccg taacaaagca ccacaaattg |
| 21961 | ggtggcttag cacaacagaa atgtattgcc tcacagttct ggaggccaga agtccaagat |
| 22021 | caaggttgcg gtcagggccg tgtttcatct gaaggcccag ggaagcagct gccccacgcc |
| 22081 | ttctcctagc ctctggtagc ctctggcatt tcttggctta tagatgcatc tgtcaaatcc |
| 22141 | tgtgtcttca tatggctttc tcctttgtct cacactgtct ttcctctgtg catgtctgtg |
| 22201 | ttcagatgtc cctttttata aggatgtcaa cccaattgga ttaagttcta ccctaatgat |
| 22261 | ctcattttaa cttggttacc tctgtaaagt ccttatttcc aaataaggtc atgtgctcaa |
| 22321 | gtactaaggg gttaggactc cagcatatct tggtggtaga cacaattcaa cccataatgg |
| 22381 | gaaggaaaga tgttgggcac ctgtaactcc tccaaacacc cacagagtgc agggtgagct |
| 22441 | gtgtgctaac acatagtcag ttctctttgg ggtgaggagg cctaggggca gggcccccat |
| 22501 | gtggggtctc tgtccacacc agcaacaata acaaccaggg aggaaagcat ctcattttcc |
| 22561 | ttggctcagt tcagcttttt atgtttttag cacaatgcct gctttgctct tccaacaatt |
| 22621 | tgggaatctc tgggagctgt gcatggaaag caaggaggac agcggcgaga aaaaggggga |
| 22681 | gtagatggag ggtcttggaa agcagagggc ctaggcaggc agagaggaca ggaaagtata |
| 22741 | gcgagcagag cggcaaattg gtggggaggt gcagaaggct gcttggcagc caggagttct |
| 22801 | tgccctggcc ctgccatgag gctgcatgtc tgtggcctag gtatttacct tctccaggcc |
| 22861 | tcagtttctc tgtaggcaag attgggaggt ggatgggtgc tctctaggat cccttcctgg |
| 22921 | ccagaataac attctcagca ggagcctaac gtgtggagca aatgggagca ctgggctccg |
| 22981 | gcctcctgca gtgagcacag cccctgttct tgtggaaaca tcttccaata gggctgccct |
| 23041 | gcctacaggg tcatgcggca tgcatctgct gcctgcctgc gctcttgaaa cagcctccac |
| 23101 | tgctcccctc ccagctcctg tctctctgca cacgcaagcg tgctactcct tttcatgatc |
| 23161 | cccattagta ttctttgacg atggcataca tctgtcttcg atcgttgtca gctctgggag |
| 23221 | gcttatgcca agcttcttga gcgtaaccca tgactgcctg ggttaggtgt tgtgagctgt |
| 23281 | ccaggaggca ggaggacgat gcatgcaagt cagggcttag ggcagaagtg cctgggcctg |
| 23341 | gcctcccctt ggactccagg agtcctgtcc taacagagcc cacagccccc tatccatctg |
| 23401 | gcctctgtaa cccctcccca acacacacac acacacacac acacacacac acacacacac |
| 23461 | acacacacac atagcccctg tgattgaggg ggccccaatt cctgttcata tcctccagga |
| 23521 | tagcccacct gcaccctcga cagtgagaga caaagttcta ttccctgttt agatgggtgc |
| 23581 | tggggacaat ggaaaggagg tgtggctctg agaagttcat gtcttgctca gggcacacag |
| 23641 | cagctgatcg ggaacatgtt gctgactcca agatgctgcc ttgcaagaag ctggctctat |
| 23701 | ccttcttttg gctgaagtgc ctttcatgga tggtgaggga tgtgcaggga gaagtgtcag |
| 23761 | gagtgagggt cagtggttag aatcaggcag tccacagagt ctgagaaagc aagacattct |
| 23821 | ctggcagtct gggggtcatg atcgcccacc ccagcccaga taaccctcac agctgtgcgg |
| 23881 | gccactagag aaaaaggagg gcatgtttgg ggcaggagag gcaaatgttt gcttatctgt |
| 23941 | gacttcttcc tccaagcatg tccggacctc cagtcaatgg tgggctgtca gtcgtcagct |
| 24001 | gaggttgagc tttccttagc aggagcactg gtcacttggg ctgggatggt tcttagtggt |
| 24061 | acaggatgca ctgcaagctt taaatgcgag tggcatcatc cccttccggt caccatggca |
| 24121 | accagaaaca ccttgacaca tttccaaatg ccctttagta gggcagtgac agcccttttg |
| 24181 | agaatcacat agaatcgcat tgattgatga gtgaaaaata aatggatggt agcctccttt |
| 24241 | tgtgattttt gcagcggcct ttagcttcct ttactcaccc cagaaatcag tgggaccctg |
| 24301 | ggagctgtgt acccctcaga cccagttgga acccagccaa gagtacttaa tccatcccca |
| 24361 | cttgtggggc caacggcacc taaccacctc aggcacggtg gacctggctc ctcagagagc |
| 24421 | tctagggaca gaggagagaa agggtctgca ttctgtttgc agccctgatc gtgagctctg |
| 24481 | ggggtcctct tccaccccca cccccacccc cagcccctgg agcaggtact cggggtcaga |
| 24541 | gctctgctga gggtctggct ctgggagggg aggtttgtgt aagattccct cccacggttc |
| 24601 | agcacagatg ggatgacaag gaccaaattc tgtttctggg ctctgatatt tgccaagatt |
| 24661 | tttaccaggc ttcctggaat agacagggaa gcagagcaag ctcccgtagg tcaagtgatt |
| 24721 | tgggcccgag ttgacccaga gtccctaaat gactgctgtg tagctaccat gagtgtgctg |
| 24781 | agtggcccat aggggcaggt atgagagagg tgctgaggga ggcagggggc ccgcagaacg |
| 24841 | gcctcccatc tccactgccc gtccccaggt ccacaggctc acagagcaga cacggtctgt |
| 24901 | gcctgggttt gctcacccac aagaggaaga acataacatc tcgctccttt tgctgcacag |
| 24961 | gataaaacga gaacagagag gaaacaggaa gtgctttgca ttccagaaag agcagcaact |
| 25021 | gtataaagtc atgcatatta ggatttgagg tatgcatggt cagaagttag aaactaaccg |
| 25081 | aatcttgtca ttgccaggaa gtttcggggt tctgtgactg gtggccactg atgttcctgt |
| 25141 | gttcctccat tccagctcct accttgactg tgtcctcctc ttcacaccta acttctttag |
| 25201 | tgaaggctcc atttcctcat ttcctgttca atgcttaaac cccttgcaat ctggcttcta |
| 25261 | ccatcgcctt atcacagacc cttctctggc tttgccttgc ctggcccttc catgatgtct |
| 25321 | tctttcttga aaccctcttt ccttggtagg ataccacggc atcctggttt ttgtcctacc |
| 25381 | tctgtggctg tttctgcgtg ctttccttag ctgacttttg ctcctttatc tgacctgggc |
| 25441 | tctcctctct ccatacactc tccatagcct attctaagtg tcccaggtct cttatatctt |
| 25501 | atctctcaaa tgcacaatta ctttgtgtta gctacagacc catatatcca gcttccctat |
| 25561 | agatacccca aatgtctttg taggctccta aactcagtgt atgctaagct gaacaggggc |
| 25621 | tccttttgtg ccccaaactt tcccaactcc agtgagtctt ctccattgtc tttcacctta |
| 25681 | ataaatggaa ccacctgcga ctctagtgtg tggtccagag acttggaagt cacctcagct |
| 25741 | tgtctctctg tcatccgcag gatcgggcag cctccaagtc ctcatcattc taactctcat |
| 25801 | aatgcctctg gagtttgtcc agatctcctc gtcaccactg ccgctacgct aatcaaaacc |
| 25861 | accattgtct cttgccatcc tccatacttt gcaaagttaa ttgggtcatt tttctacttg |
| 25921 | aaatcttata atggctctcc agtgcctctg agtccttgtt ttttcaacac tgttcacact |
| 25981 | ctccccacct ctctctcact cataccccat gcaccagcca tcctgggttt tgctgttttt |
| 26041 | gtttcccaga atgcacaatg caccttctgg cctctgagcc acagcacctg ggtatttgct |
| 26101 | cacactactg cagctctcct cttccctgcc accacgcctt tcttgcctga ctgttagtat |
| 26161 | gcagcagtgg ccacttgagc atgaccgcct ctgggaggct gtccctagtc ctctgtcgca |
| 26221 | ttctggggct ccctatcaca cactcccatt gcatgctgca gcatcctcag cacccagcat |
| 26281 | tccttattgt agttcctgat tcaacacctt tctcaggaga ctctggactt cttgaaggca |
| 26341 | ggaacaattc ccacttgttc ctagtagcat tccaaaccac acgtgacagt gtctggctta |
| 26401 | taataagcag ccaataaaaa gttgatgaat gaatgaataa gtgaaaacag aaggtgtttg |
| 26461 | cctgcagaaa tctggaataa gatcaaagat cagagctggg attaaggaaa aaacttcctt |
| 26521 | ggggtggcac tatgaattcc cagaacaggt gactaaccct cattcacttt ggcaaatgtt |
| 26581 | tatcccatgc cacgcaacca ggcaaaaagt tgaatgaggt ttaatccctt cccacacgga |
| 26641 | gcttattcct tctttggaag tcctttaaac aagctctgaa atgattttgg caggtagaca |
| 26701 | aactggtcct catttctctg tgaccagtaa gtagggaaag caagcacaca tacacacaca |
| 26761 | cacacacacg tgcacacgca cactgacaga caaccttgct cactcacatg ggcatgccca |
| 26821 | aacccttctt ctattttata ggatggtaac tcactcttta gtttagactc ttgacgtgcc |
| 26881 | atggaaaatc ccactcgccc tagaactggg ggccgggcag gtttgactgt aacaacgaag |
| 26941 | cctggagctt actctttgct gattggcttt cctttctgtc tccatttttc ccctggtgag |
| 27001 | cactgcagtt gtgttcttcc tcccaaaggt aatgcctggt ttggctcact aaaacctgtt |
| 27061 | ctttctgtac cgagagctca tcttctcttc ctcttctgga ttctcaaatg agatgacgtc |
| 27121 | agaggatgga ggccaaccac acccttcctc cttgaccctg ataaagtttc ttggaaaccc |
| 27181 | tatactcaga ggcagccaat tcttgccagt ggaagagtga aaagagggct tgggaagctc |
| 27241 | aaggctcagt gtctgtcccc aggtccccca gttaaagaca catctgtcct tcactctcaa |
| 27301 | agatgttgcc attgctcccc tgctagagtg acacactgca ctccctcctt cccttcacac |
| 27361 | cccagcaaga ggctatttcc caggggtctt ataagcagat ttcatcttct cttgtgctgt |
| 27421 | tttcttattt caattatctt cagggaggaa cgtgcatatt gcgtcattgc ctggctgtga |
| 27481 | aatttcattt ccatttcttt acacctgcag ttgcaatacg agagagaaaa ggccagagct |
| 27541 | tagcggatgt cctagacgca ggttatcaag gtgctgtggc tgtggtttcc cggaaaaggc |
| 27601 | cttggtccca gagcacattt tatcagcagg accttcgagg ggctgcgttc cttcaattgt |
| 27661 | tttctctttg gggtctctgg tctccagttc tttcttctct agcatgtgag atctgtgctt |
| 27721 | ttgattcatg cctttaagtc tgacattgaa aaaatatctg atttgccatt ccagatgctc |
| 27781 | gtcctcattt gcaaattttc ctaaagggcc agattgtcct ctggcctttt cccttttcct |
| 27841 | ggtcccacct caccaccctc ccactggggc ttcacagagg cagagctagt ctcctttcat |
| 27901 | tttttaaaat taatagtctt caatttttag aacagtttta agttcacaga aaaattaacc |
| 27961 | agctattaca gagttaccgt ataactcctc cccctcactc cccagttttc tccattatta |
| 28021 | gcatgttgca ttagtgcagt acatttgttg cagttaataa gcaaatatta gcccatcatt |
| 28081 | attaactcaa gtctatagtt tacattaagg tgtattcttt gtgttttaca gttttatggg |
| 28141 | ttatgacaaa tgcataatgt tatgtatcca ccattatagc atacagaata gtttcactgc |
| 28201 | cctaaaactc tcctgtgctc cacctgccca tccctcctcc ctcctctgcc accaatccct |
| 28261 | ggcagccacc agtcttttga ctgcctagag tttcgccttt ttcagaatat catagtagtt |
| 28321 | ggaataatac agtgcgtagt cttttcagac tggctccttt cacttagcaa tatgcatttt |
| 28381 | aagtttcttc catggaaact ttgctttcat ccttttatca ccacaaggcc agtcatccaa |
| 28441 | ggaatttctc catctctgtc tgttcctttc tagttctatg tgtgccactg cttggcatag |
| 28501 | aataggtatc catttaatga acattccctt tcaccacctg ggacaccttc ccagggataa |
| 28561 | caaaaataaa accagctagg tcaatagcag agcccccatc ccagttttaa cctcattctc |
| 28621 | ccctctttcc acaataaact ggatcagaac cagcagctct gtaagactgc atttctttcc |
| 28681 | cttaatacca ggccccagag agcatttgat tccttggcag agaggtgtag gcttaattaa |
| 28741 | tttttctcct tttttctttg aacatcttgg aacacacaca cacacattcg catttatgca |
| 28801 | caattgggtg tatgagaatt ttaatggcag gtggtgttag cagttctttt cctcctgata |
| 28861 | cagatcaggg tttttccatc tgggcctttt agcagggcct atgaatattg actttctaac |
| 28921 | cacttggatt tgggtggagt gtgcagagtg ggattggggg ggaaggttca agggagaact |
| 28981 | atacttatgt ataaatcaca tgtgaaggga gttttgaagt cattattgct tcaggatgtg |
| 29041 | cgaaccataa ttatttttta aggtcttgat ttgcccaaag agcatttccc agggttgctg |
| 29101 | ctccaagcat gacgtctgtg ctgtcaggag gtgcagcata gtctgattcg agtttaatcg |
| 29161 | ctttaaagga ggccctgggt aggatctggt ctctaggttc tcagctgtgg tcagtcctcc |
| 29221 | atgcagcaaa acatccagat gacttagatg attaagacag cagacttaaa gtgaagaaga |
| 29281 | gattttttcc cttattcttt ccttttatta ttagttttta aatggttggc tacatgggct |
| 29341 | gttggtcatt ctccatgttc tctgtgctct cctcagctct ctgctcaaaa caggctgcac |
| 29401 | cggcctgcct aaaccctgaa agcaacttct cagctgccta ctttctgcct tttgaccccc |
| 29461 | aagccaatcc ccatctcctt accaccctcc cgccatgtcc tcatacacct gcctctcctt |
| 29521 | gacttcattc ttcatgctcc atcagcaaca gccctctgtc aataatgatt gtcccaggga |
| 29581 | agtgtattca agggtcacat aaaatgtgcc ctctctatgt gttgagaagg ttttctgtcc |
| 29641 | ccaaaggagc tctctggata atgaggaagg ttgaactggg gcagcctaca ggaagaagcc |
| 29701 | cttagaaggg aaacctgtgg cataaaccat gctgatccac gactcttatt ttggaatagc |
| 29761 | tatttaaaaa gaaatatgaa gaactcgtaa gacttggaaa agaaaactag agaatgttga |
| 29821 | aaatgtccaa gggttatgtg tatgatgtgt atggggaaat tttaaaagaa tgtggtagaa |
| 29881 | aactgaattt gtggtaaaat gttgtcacag gacggcctgt tctttcattg aattatgtct |
| 29941 | tagtgcaggg atgacaaata aatacagcaa ctgtgctgcc attctcacat cttttcctac |
| 30001 | agtaggcatc actaatcaat tacaacattc ttttccactg tcatgccttt gcctacagca |
| 30061 | gacatcacta atcaatcaca gcactctctc tcattgagtt tcaaaggttt tttaatcctc |
| 30121 | cacataggtc tctaagtagc tatataccaa ttactttgat ttagaacttg gattaccaat |
| 30181 | ttgccatctc tagtttaatg ggaagaacat taggattaga gtcagaatac ctgaattcaa |
| 30241 | gttactgacc tactacttaa taataattaa ttttaataat tctctaacct cagctttctc |
| 30301 | ctctataata agaaataatg cttaccacag aggctcgagg taaggattaa gtaagataat |
| 30361 | atataacatg taagataatt tgtgtgatgg ttcctagaat agtacctggt gcattgtaag |
| 30421 | cacccagcaa gtgatagcca agtatgaatt tgcagcggca tggacacatc atgccacacc |
| 30481 | ccagatatgg aacaatagtt aggattccat tcacctttgc ttcttttaac atcctcagtg |
| 30541 | aaggcagatg gagaacagct ggaccctcta attctacctg actttaactt cacattctta |
| 30601 | agatgcttat taaatctctc tttcctgaac taatataatg cttccttgtt accctggaag |
| 30661 | gacagagtta acacccatgt attatgacat gatggatgtt ctttttgcgc acactgcatg |
| 30721 | catagccatg tgccaaggcc aggcctgcct tagctttttg gttctccaga gcagcttcac |
| 30781 | tgtgactgaa gagagccagg gaagacatct tggtagagct ctttatatga ctcttttcag |
| 30841 | aatgtttctc actgatggag atgacagaaa gctaggatga tttgcaggca ggaggacagg |
| 30901 | cttctttgga aaaggtttca ccatagatta cctcaatcca gggttggaag actggaaagt |
| 30961 | ggtctgcagg gcatcccagg ggatgccttc ttcaagacaa tattagacta gcattggacc |
| 31021 | ctgcctccag actagtaaag ggttattttc agagcagcaa caccagagaa catgttttga |
| 31081 | gcaaatcaaa tccttaaaat catgatttca tacatcctga gatactagtg acttcaaaat |
| 31141 | gcctttctca aattatacat atagtgcctt ctgaatcaac tgtctttttt ctgtccaaca |
| 31201 | gtataaacat aatctctgcc ctcacacagc ttagaaactg tcatgagagg taaacaaata |
| 31261 | cacaaatgac aaggtaaaat agaagagcta caaaagagat catgaaaata tccagggagt |
| 31321 | tcgaaggtgg cagataacaa aaggcttcgt ggaggaggta gcctttgtgg catttgagat |
| 31381 | cagtcctgca gcagggttat aggtggacag gcagagatgg ttgggggtgg gcggtaggag |
| 31441 | ggacagacag aaggaaccac aagaatgagg atatggagaa aaagaccact gaagggtgca |
| 31501 | aattattaag gatggattta tgtaaaggat gtggcctaag aagaaggact cttctttatg |
| 31561 | atgaaattag gttttagagg cagaagtctg gtgcttcccg gtactaaaat gaccccagta |
| 31621 | tggtgtctgc agaatactta gataactgca tagatggttc agccttcttt ccattatacc |
| 31681 | acactactgg gttaccaact tgctgtgtga ccttagataa gcaactacct ctctctgggc |
| 31741 | ctcagttttc atatctatga aattaggagg ttgaattatg tcttggcata ataacgaaat |
| 31801 | aaataagtga tttattttta tttctttgtc attcctcttt gaaatgggag tgggaataaa |
| 31861 | gtggtatttg tttccacatg aaaattaaag ccagggggcc aattctggac ttggcttgag |
| 31921 | ctgtgctttg tctgtttttc tgcactggcc cagtaccctt tacactggtg taactaactc |
| 31981 | cctggaaagg gatgcaggta gacagcgtga ctgttccttc ctatctgaga ggccccaaca |
| 32041 | gattctatat tgcaggacca ccaacttgga atttcagctc agttcagcaa acacttttga |
| 32101 | ctggttcgtg tgctagacat tgtgctgggc ttatgtgggg tagagatgcc tgctaatcta |
| 32161 | aggctaacag gaagacaggg aagtggtttc cagacctgat gcttatcaga gtcactgtgg |
| 32221 | aagaactttt tagaatgcag acagccccca acttaacagt ggttggactt tcaatttttc |
| 32281 | accttataat ggtgcaaaag tgatgggcat tcagtatgct tctcaacata caaagaggtt |
| 32341 | gtgtgaaata aacccatcat taattgaaga tactgtcaat caaaaatgta cttttgactt |
| 32401 | acagtgtttt caacttagga ggggtcatag gttgaggagc atctccacag attcctgagc |
| 32461 | cccatccctt gatgatgatg atccagtgag cctgggtggg gcctgagaat ctgcatacta |
| 32521 | actgctgaag gtgagtgtga tgcagggcca agcttcagaa cctctgatct agagaagatt |
| 32581 | ctgtgtcatc acagctcagg gtgaccatgt tctctcttac ttgcacttac atgaattcat |
| 32641 | atgaattcag tattacatga gtaaattatg attatatgaa tttattgagt atcctatgtg |
| 32701 | cttgatatag gtgtttacat cccagggttg gggggtagag atgaagaata taagccggac |
| 32761 | tatgttaaga tcttcacttc tcagcacctg aacaactggc agcctcatta ggaaggcaag |
| 32821 | tcattcacag gtgaaatgac atcatggtcg tcttcttcat tcttctcttc agcatccatt |
| 32881 | taagactcac attttatcac cagagactga aaagagccac ctaaggcagg caggtcaggt |
| 32941 | ggtgttatct ctttatttcc agatgtggag gctgaggctc agagaggtga atccatatgt |
| 33001 | ccaagctcac atctcccgcc ctcagtccag ggcttctccc cacttcatgg gagaagcatc |
| 33061 | ctcctcccca gagcagtagg ttctggagct gggagaggcc actgtgggct ggattgttgg |
| 33121 | ggacagcttc agggagagcc cgattcaagg caatagagaa ctttggctgc aggccgttgc |
| 33181 | ctagaatagg gcagctgaca cacctttgat ctggaatgat tcctgctgct gagaatgagg |
| 33241 | ttttttatat ctggattctc aggtagtaac accacgacaa cgtgtgtttg tgttatttca |
| 33301 | tctgcacagc attcacgtgt agcagagaga aggtagttta ttccccagaa gtttaccagt |
| 33361 | gggaaattga ggtcaaaggt aggactttcc taaatgaaga gaactactat ttattggatg |
| 33421 | cctcccatct gccaagcagt gtgcacagca ggcatatatc atgaaatgcc tcatttattc |
| 33481 | ctcataactg tagcctgatt ttacattcgg caaacctgag gcttacagaa atcatgtggc |
| 33541 | tcgcactcag actgctgatg gccacgcaag gctttcagct tctctgattt ccaggccccc |
| 33601 | atccccacca cactgtgctg tccagctcac ctggtggaac tggatccttt gagttccagg |
| 33661 | ccagggatcc tactgagctc ttccatcagg gaaaccatag cagtagcagc tccaccaagg |
| 33721 | acttggcatc tatcttactg cagcatccgt gcctgtctaa tggaaccatg taccaggagt |
| 33781 | agctacccaa gaaacatttc caccagaaac ttctctttat agctccccat cgagcctcag |
| 33841 | agagctgata ggaattgctc aagaccaccc aacttgtaag tggtagagct gggactaaaa |
| 33901 | ggcaagtccc ccaactttca gcctcactcg tgcccaatgt gtctcagcct ccctgaagaa |
| 33961 | tatcagacca agatggccag aaaaggaacc tggaagggac gtgtgggcgt catgcagccc |
| 34021 | ttggcacagt cttcaggctg agctgcctcc accttgtcat ctcatcagag ctttacatcc |
| 34081 | atccttggga gaggccagct ccatgacctc tcagtgtcat ttaggatctt cttcctaaac |
| 34141 | ggctaaccca agtctgtccg ctgcatccta cagtgagatg cactgtggcg agagcagcta |
| 34201 | ttggcattct gcttacgtgc tgtttccaga ggtaaactca gtataaatgg atctacagcc |
| 34261 | tgtcccattt tgtacagtcg agttctaaaa ccagcctgca aggataatgc tataaaaatg |
| 34321 | tcctgccagc cccaagggtg tcttctacaa caagttcttg ttccctcata attcttctga |
| 34381 | caaattcttc ttttgatcca gactttccta gccatcattt cattccagaa gtggcatgtg |
| 34441 | tgtgcaaaca ctcatgcttg agggcgggac aagcaaaggg ataagagggg aaatgggaac |
| 34501 | tcgaaatctg ctcaaatgtg gtaaagaaat atatccagaa agtactgcct actcaccaaa |
| 34561 | atactatttg ctttttatat tctttcctga gtagaatttc ctgttcaaac ttgaaaatga |
| 34621 | aaattcctcc acttcaaaat gaacaggcaa gaaatgctgt aggctgggtt tcccggggag |
| 34681 | gggggactga cagccatctg ccctgagact gactgtcaaa tctgaactct gtgtacttgt |
| 34741 | tagtgttgtt tatgggaggg gtgagggaag ggaggaagca acagggacct gctaacccta |
| 34801 | tgaattctcc ctcataccct taaaaagtcg ggtgcttggc cgggtgcagt ggctcacgcc |
| 34861 | tgtaattcca acacttggga ggccagggcg gttggatcac aaggtcagga gttcaagacc |
| 34921 | agcctggcca agatggtgaa accccgtctc tactaaaaat acaaaaatta gccaggcgta |
| 34981 | ctggcaggcg ccggtaatcc cagctactcg ggaggctgag gcagagaatt gcttgaaccc |
| 35041 | aggaagtgaa ggttgcaatg agccgagatc atgccactgc actccaacct gtgcaacaga |
| 35101 | gcgagactcc gtctcaaaaa aaaaaaaaaa aaaaaaaaaa aaaaaaaaaa gtcgggggct |
| 35161 | tgccaagctc gctttttggg gcaatgggag ggaaatttta agagctgatt tctgtgatct |
| 35221 | ttatcagacc tcttcttctg tcctgcccac aggtcagtga tgggaatgat agtattgcat |
| 35281 | taaaagagaa gtgattcccc agatgtggac atctcagtgc ctgggaggca gacgtcttta |
| 35341 | gagtgcttgg tgttcctccc catatctaac ttacctgttc ttaggacata ttcccttgag |
| 35401 | tgtcttttat tatttcgttg attgattaag ggttacaaat ttgttacaaa agcagcagaa |
| 35461 | attttgaaag ataaaacagg tagaaactgt tatttaattg gaggcacaaa agccagttcc |
| 35521 | agcttgagca ttaactggct gtgtggcttg ggcagctccc ctgcactttt gggaccatgg |
| 35581 | tttcttcagc tctaaagtga aagctgagag ttcctctgaa tgctaaaatc ttgcttctct |
| 35641 | gtgattattg ttttttaaat gccgccgtca tttcaaaaca catacatggg gcagccctcc |
| 35701 | tcagctgcct gtcctgttct cttccctcct tccctttgtg ccatcagcgt ctcccctgga |
| 35761 | tttatcgtgc tgtactcctt ggacaccctt gaaagtggag agagaataat tctcattccc |
| 35821 | tttccccaaa ctctgtcctg aagcaccact ccccacctcc ctaccatgcc tgcccctcca |
| 35881 | ccccccacat tttccctctg atgagatttc ctctctgagg gaatattttg gtcctgttct |
| 35941 | ctgttcccaa acaaattccc caggtccttt aaatctggtt tcaatcaaca taatcacagt |
| 36001 | ctcttcgatg ctatcaactc tgccttgatt tggttgtgtg attcatgctg tgtcctccac |
| 36061 | ctcctcaggc tgttttatat atacacactt aagtgctcat atatatgtgt atatattaca |
| 36121 | caggttgtat gtatatgatg tattgaaaga gagagatgtt ttccaccgtg cctgtaatta |
| 36181 | ctttttatct tcctccaaat cagtatctta atgcatatca cctaaactgg attcattttg |
| 36241 | agaacactag ggcctttttt ttacttttca agaacagcta tctaattctg ggccaaaaac |
| 36301 | tagttatcaa atgagggcag gtaatggagt tgtatccagc gagggggcat ttgcttctcc |
| 36361 | atatgataca ctcctcacca gtaccaaggc gttttctctt cagtcttcat gcctatgctg |
| 36421 | ttacgagtcc tttctcctat ttgaaagaaa agatatgagg cagcccaggg aaagctctga |
| 36481 | tggaggctat aaagacaata tggatgtaaa ctaaaaaatg gtagcttaga gcttaatggt |
| 36541 | agcctctcag gacactcacc acacctgaat tctaccagcc tgacgccaat gctgctccgc |
| 36601 | ttatgctggg atgtagtgac acaggacact ctgctggggg atagggatga tctcccccca |
| 36661 | ccaagtgggt catcaacact catcactcag aggggaggtg ggataccacc ttgaacagag |
| 36721 | aaagcggctg ggcatggtgg ctcacacctg tagtcccagc accttgggag cccgaggcag |
| 36781 | gtggatcgct ttagcccagg agtaagagac cagcctgggc aacatggcaa aaccccatct |
| 36841 | tcattaaaaa tagaaacatt agcctggctt ggggttgcac acctatagtc ccagctactg |
| 36901 | gggaggctga ggtgggagga tcgcttgagc ctagggggtc gaggctgcag tgagccatga |
| 36961 | ttgtgccatt gaactcctgc ctaggagaca gagtgagatc ctgtctcaaa aaaaaaaaga |
| 37021 | ggaagaagtt gctgaatccg tcttataaat ctgtaacaga aaattatggc aagttgtggc |
| 37081 | ttttaattgc cagagctggg cacttaagag agaaaagggt ttttgtgaat tccaaaaatg |
| 37141 | taattgtatt cattgaacac tgaagtcagg agaagtcaga ccataatgac atgggagcca |
| 37201 | ttttcgcaac cagcagacta gaaggggagg gtttggaagg gtggcggcag agcttagaca |
| 37261 | cctgctgcga agggagagaa gtggggacag aaagatggcc tgtgtagagt cccatgagaa |
| 37321 | agacagaact gcactggcag gcatctttag gggcccaggt cacaatcatg gggccggtgg |
| 37381 | acagtctcca gggcactgtg attgtcacag tgcacagcct aatggggaaa attgcacagc |
| 37441 | ttcacttaaa tataggtgac atatggacgt aagaattcat gattaatcta gaactaaccc |
| 37501 | tgaccagcaa ggccgaatga agaaaaaatg tagaaacact gaccaaacct tcctcaaagg |
| 37561 | tcacagatct tagggaatgt gtctctcttt cacatttcaa aataacaaca atttttgaaa |
| 37621 | tatgtatttt ttaacattta attgttttat ttggggataa ttttagactc atggcatcca |
| 37681 | ctgttttttt agttacccac atatttgatt cagtaagatt ccctgtattg atacaaaaaa |
| 37741 | agaagaggga ccctgtttcc ctttgacact gttacttttt cctgagctca ttgtttgttt |
| 37801 | gtattttgtt ttgttttggt ttggtttttg agatgtagtc ttgctctgtc acccaggcta |
| 37861 | gagtgcaatg gcgcgatctc agctcactgc aagctccgcc tcccgggttc actccattct |
| 37921 | cctacctcag cctcctgagt agctgggact acaggtgccc gccaccatgc ctggctaatt |
| 37981 | ttttgtattt ttagtagaga tggggattca cggtgttagc catgatggtc ttgatctcct |
| 38041 | gacctcgtga tccacccgcc tcggcctccc aaagtgctgg gattacaggc gtgagccact |
| 38101 | gcgctcggcc tcctgagctc attttaagag agacttctgg cctagaggtt tgaatgagaa |
| 38161 | gaagttatac agctgggatt cttccctttc tctgatatga ggacaggagt tctctctcat |
| 38221 | ctccgccaag agcaggaagc tggagtaact gccacaagct ccaggaggga gtgtctagaa |
| 38281 | catccacgtt ttgcagcagg aaaacacccc ctcacgctga agtttgattc ctgaatcctg |
| 38341 | tgtcgcagtc taaatgctga ggcagaaggg gacatccgtg ttcctggggc attccacttg |
| 38401 | cagtcctggc tgtaacccga gtgagccatc cgtgtagttc ctgttgctaa gtctcccctg |
| 38461 | ccacctcttc ttcccatggc tgcagggcag ggggccatgc cctcctcttc atttcctgtc |
| 38521 | cctgggtgag cgtgccccct gccttctccc agatctctgc tgtggcagct tcacgtggga |
| 38581 | ttcagcactg tgtctccttc ccctctgctc ggcctgccca tacctgtcca gcagagctgt |
| 38641 | aagaccagaa gacagagcat tccccttatc tatgaagtca aatgcatgtg tggaacatgc |
| 38701 | cacccagcct gcagtctctc tactataaaa tactgctcat aagacaaatg tgtggcccag |
| 38761 | atgatttctg ataaagtcta ttattttgaa atacatatgt atgtctctca gccactgata |
| 38821 | cacgcagaag ctgcacatgt tgtcaacacc tgctttgagc tcctttcctt cccacccttc |
| 38881 | cttttggcaa tgcaagtttc cattcatttt ctgcattact ggtctcctct cttctcccct |
| 38941 | actactagat cttacaataa acatttgaaa tagtttattt gtccacagtg tgatttctgt |
| 39001 | tgagaaacat gggctcaccg acttttgggt ctcttctaac actagaaatt cctctggttt |
| 39061 | tttagactat ttcaagggct attaatgtgg aacagacggc ctttagaaac agcaatccac |
| 39121 | agggggcccg gagacctgga ttcatatttc tcctagcgtc aactagtggg tgaccttaga |
| 39181 | aatgtcattt tccttgtcgg gctttagttc ccttatctgt cacacagaag cactgtgtga |
| 39241 | gtttgggaaa ccaatactat gttgaaatgt acaaaataat cttaaagcac agatattctg |
| 39301 | ttccctccaa gaatacatca aacaaaggaa ctgacattgc aagaagattt tgaggagatg |
| 39361 | gctggatgca ctggagcagg gattgctgag ggaagccagg ccctcacctg gagcgtctca |
| 39421 | ggagaggcag cttcggtgct ggctgtttat tgcaggcatc tctttctgtg tctgtgcgtt |
| 39481 | taggggctct tctttggaga taagaaaagg gttctggatg gagggcagtg aagaacagtg |
| 39541 | agaacttaac atgaggatgt ttgtatagag gggaagactt ctggacagtg gcttgacttt |
| 39601 | gctcactggg catttccttc tggatctctg tagaagtcag ggacagatct cctcgtgcat |
| 39661 | atctgtctcc cagagacaga tctcttccta gcagaaagta gaaagtgggc ttcaggcatc |
| 39721 | ctggaagttt tctttcttgg tgggtgataa aagggcttgc agagagagga gaatcaaatc |
| 39781 | tcccacatgt gcatcatgcc tgcgagtctc atgcagagat gtcttatgtt caacatagaa |
| 39841 | agcaagcctg gcagccccaa gaccttcctc tgcacaccgt ccatttttac ttggtttcat |
| 39901 | tttgataact gtgcggtctg aggtcctggc caagaaagca tcacctggca agaagtgtat |
| 39961 | ttggccaatg gtaaggttac catctctgtg taattaggct ccgtaaagct ttgtttttaa |
| 40021 | atttattaat gggaatgatt tgacattcct acacactgac attaccctca tggaatggat |
| 40081 | aagaatctca aggcttgttg ggtgaaagaa gggcagtgtt tggtgtgacg ggaagggaaa |
| 40141 | gtataagcag gcagctcgtg cgcatgagca tttgggaaac agaacagaaa tcatagaatg |
| 40201 | gcaggcttaa ttctagctct gtcacctact ggctgtttgt cattagaaaa attatttacc |
| 40261 | cttcgtgaga ttcagtttcc ttacatttaa aataaagaaa atattcgtcc tcatattgaa |
| 40321 | atgaattggg ctatcatgat caactttaaa atacaacgaa cagtataaat gtcaggaatt |
| 40381 | atatgacatt cgggacctcc actgccaccc tcactttctc cctccagtgg tcacttactc |
| 40441 | tctgtccctc tttctgggtc agagcttctg ttactccagc ctgggcctgc cttaagtggg |
| 40501 | gacatgtgct gatccctcac aatgccgggt gacaaggagg gttttcaagg ctggcttgac |
| 40561 | tgccactgct ggtctctctt ctcatttgca actgtcttct ccccgctggg ctcagtgttc |
| 40621 | ctgggagggt gatgctgagg gagaaagctg tggcagaggg acgtggcagg gtcagagacc |
| 40681 | actgattcgc aggagctggc ctcagagcta gcctttttgc attgatctag ggaaccagtg |
| 40741 | atcatagata tctatgttga cgcctgtgtc aatttatccc tagagccatt attcagtgaa |
| 40801 | tttcctaagg ggaaaacaat tctccagcat tatttttctt tgggccagga gagcactttc |
| 40861 | tctgagtttt actggcaagc tagatatatt cttgaaaggc tccagcagca gagttcccgt |
| 40921 | cttgtttagt tcaaaaacag ctcctggccc gtctctaaat ggtcttgcta aaaacatctc |
| 40981 | ctcccaccca tagacctgaa cttaagcctc agactgctat cctctctttc tgccactgtg |
| 41041 | agagacctat gcctcttttc tcattggctt ttgcctgccc agccctcctc agatccttgt |
| 41101 | acatccctag gaagtacatt ctttccactc cacacacata attgtgcaac ttgtgctagg |
| 41161 | accgcatgag gcactggagc tgcagatagc aaggaaatgt gggccctgcc ctcaggaact |
| 41221 | tgaaatccgg tagttaccta gagctacact gagttccctg acgtggtagg aagccctccc |
| 41281 | agagccttgt ccgtgcttag acgttgcctt cacagaggtg gctaaggggc attttgtccc |
| 41341 | tgccctagtt tttacaagtc ccctggatgt taactcctac ttgcttttat ttgcaggttc |
| 41401 | tccagtctta tgcattctct cttattccta aaaatttcca atccagtgct gatagtatgt |
| 41461 | tagagctgta gggccaggaa accctgctgg gggaatcatc gtctagtagg tggagtgtga |
| 41521 | gagagaggag actcaggcca gaggggcttc tgagttctgg gcaagtccct taccatccta |
| 41581 | gtgcagctat ccttctccat gtcctactga gctgccttcc ttcttgcttc tcattcccaa |
| 41641 | agagaggcaa ccatgccatt ctgggcagtg ggcaagggct gctggaattg agatttttat |
| 41701 | ttttctcttt ggcatctgaa tccttccttt gcagttgctg acatgcagtt ttgtgtgaga |
| 41761 | tcacccatgt catccatcat cttcaggaca cccagaaact cctcctacct ctctcagcct |
| 41821 | cacatgcgca tcttcagtcc ccttctgatt caccctagtt ggtctattgc tcttggctct |
| 41881 | gaataaatac ctttaaaggg tacaggtatc catatggaag gccttcagag aagagagagc |
| 41941 | atggaattta ttttccaaag tgggacactc gagagtgaaa gggtgagctg acaataatga |
| 42001 | tggcaggaca aaagcatatt tcaggcttct taagagcttc atccctttga tccagtgact |
| 42061 | ttatttctag gaatctttta tggagtacct accatgagcc aggcaccatt gcaggtgcta |
| 42121 | aggatatcaa aacacatcaa aagagagcat acagttaagt ggagcagaca ggcaagaaaa |
| 42181 | aggatggttg taaaagaatg cggtaagtgc tgagaagtct ttctcagaga cgtgatgtat |
| 42241 | gagcaggctc agacagtcaa atagtagtta gccagggaag gaaagggaga aattgccttc |
| 42301 | ctggcacagg gagttgtcag gggagccctg gcagaggtga gagtatacat ggtgtgtttg |
| 42361 | gaaaacccag gtccctcgct gaggctgctg tgcacaggga gaggagggct ggcaagagat |
| 42421 | gaggctggca agtgaggcac ccaggagcca gacgatgaag ggcagacaat gctgaggaat |
| 42481 | tttagcttga tcttggaggt tatagagaag aacagaaatt tctccagctg aggaatgacc |
| 42541 | caattccatt ccagtgcggg gaagaaatga tagggaaggc tgggagaggg caaaataggg |
| 42601 | tctcggaaac catttaagag tctattgaag taatccaggc gaaagaggta gcatggactg |
| 42661 | tagagaaaag gagggtggat tttgagaaat aggaggaggc agagtcaact ggaattatta |
| 42721 | aagatcagtt caatgtgggg gcagggaaga aaggaagaaa gaggactcaa acatgatttc |
| 42781 | tggtgctgcc ttttcccaag agagaaaatc cgagaggctg gaggaggagc agggtttggg |
| 42841 | ggaggatggt gacttcagct ttggatatag cacccttgag attcctctgc agttcaggtg |
| 42901 | gagatgtcta gtaggccatg gatgtcccat ggatgtctat gggtttggag tataggacgg |
| 42961 | aggtctgtgc ctggagatgt gagcatagac gtatctacac atcagcaggg ttgatgagat |
| 43021 | tgcacagtca tatcctcaga tgagaaggga aagcagaatg ttggagaaga atatggaaag |
| 43081 | aatgcaaggg aagtgctggt ggtggtagga ggaagaccaa gagaggtgcc gtctggagga |
| 43141 | gggatcggag tgtccagtga ttcagagagc atgagggagg aggggctgtg gcacacctgc |
| 43201 | ctgactttga aatgaagaat ggtattctag gtggggctgt ttttttaggg acctggtgca |
| 43261 | ggtggggatg atggacatag ggaattctac tctctgggag acagcatgtg gctgggatca |
| 43321 | gatagttaca aagttatatg acaactattt tatttcattt ctctaatact ggcaacatct |
| 43381 | gaacattatc tacagaggaa ggaaaacctt tatggagggg taggttgaaa atatgtctgg |
| 43441 | gccaggcatg gtggctcaca cctgtaatcc cagcactttg ggaggctatg gtgggaggat |
| 43501 | tgcttgagcc caggagtttg agaccagcct gtgcaacatg gcaaaacccc atctctacaa |
| 43561 | aaaatacaaa aaaatagctg ggcttgtggt gcattcctgt agtcctagct acttggaagg |
| 43621 | ctgaggtggg aggcatcacc tgagcccaag aggtcggggc tgcagtgagc tgtgattatg |
| 43681 | cccctgcact ctagactgag tgacagaatg agactctgcc tcaaaaaaga aaaaagaaga |
| 43741 | aagagagaga gagagagaga gagaaggaaa gaaagaaaga aaaagagaaa gaaagaaaga |
| 43801 | aagagagaaa gagagaaaaa gaaagagaaa agagaagaaa agacaagaca agaaaagaaa |
| 43861 | agagaaaata cacctgggat ggaggccctg atggaagaag gtttctgagg aaatgggtgt |
| 43921 | gggtcccaga gtgtcagtat agggcctgtt ggagagcgag tatgataagg tcagccctgc |
| 43981 | cactcagaag aaattctggt tatactcaca gtaatcacac ttcctggttg acccagggca |
| 44041 | gtcctaccgt gcaaccatta ccccaacaaa attattgctt acaccccttt tcacttttga |
| 44101 | aagtttaggt gataaagtat ctggtctcct cagagtatac atctaaggag attcgtaggt |
| 44161 | ggagttagac agctgaggga gctcctacct gaaggactct gtgcagcagg aggcaggatt |
| 44221 | ctctgtatga tgttccaaga ggcttgagga gataggagca gctgtgaggg gagggaagac |
| 44281 | ctgaaatgag aaaaagagca aattgttgat ctacagtaag ggctgagcag agcagatcct |
| 44341 | tgagaacatc aatttgctgg gcaccaagta gttcataggc cgcgtttccc tgtctttagc |
| 44401 | agcctgcgtg tctgctggga gaatagatgt ataaactgat ctgaggttgg agctgcacag |
| 44461 | ggcagatgtg gccagagggt aaggaagtgg ctggagtggt ggatcatgag aatagcacac |
| 44521 | ccgtagtaag ctctgcacat ggagatactt attgcagcac tgtttatggg agcagaggat |
| 44581 | tcaaaatagt aaatgtccaa caagggggtg tcatccggta aattatggca aattaacgtg |
| 44641 | atagaatatt atgtggccat caagattttg tttaggtaga attgtaatat aaggagagaa |
| 44701 | agcctagatt gtaatagtaa gtgaaataag aaagttccag aatgatgtat attaaaccat |
| 44761 | cccgcctata atgcatagag atttttttat ttatttttaa aaggttgggc acaaatattc |
| 44821 | cataataata cctgtgggag ctgatagatg atatattttt gtatttttaa atttttcttc |
| 44881 | aataagttca ttatattact tttataataa ggaaaaaaca tctctctagg aaaaattatt |
| 44941 | taaggaaaaa ataaacatga aaaagggtgt ctgtaatcac agtttaattt ggccttctca |
| 45001 | gaaaaccatt gtagggagtt tttattctct agtttcccca gggtggggaa gatgagcagc |
| 45061 | atggccctag tgtttgagaa cgtgggtttt gatatcagac atgcctgggt ttgaatccca |
| 45121 | gctccgctaa gctgagctat agtattgcca tggtgtgatc tcaagcaggc tatccttatt |
| 45181 | tatgaagtgg aataatacac atccccagtc tcttctttat aattccgaaa cccaaaaagc |
| 45241 | cctgaaaacc cagagtcgtt tcttaaagtg acagcaaatt catttggcta taaaacctaa |
| 45301 | cctgaattga gctagtcttt atttcacttc agtgaatata attttgcagt ggaaatatta |
| 45361 | acatgtttaa tcattggatg ttcgcccaga tctcacggag ggtatgagat aatacgcagg |
| 45421 | gttcaggtaa gttgtgaact acaggatctg cgtcataggg ttattgtaga atgaagtcag |
| 45481 | gttgtgattg cagggttctt agcacagttc ttgccagaga gtaagtactg acaaagtgag |
| 45541 | ttatcatcac tattgatgct ataattattc gtgagccttc actaagtatg gagtgcatga |
| 45601 | tttctgcttg cccctcttcc ctccatttcc ttctttattg ggatgtttga gaagattaat |
| 45661 | gtatcacata taatgagaat ttaggatggc attgtttagg acacaggact gcaaaaaagg |
| 45721 | gaagttccag cacagatatt ttcctgcctc ttttcttatt ccagatttct cactttctgg |
| 45781 | ggaattagct gtaggatata atgcacatta acaggacacc caaatacttg aagagctaat |
| 45841 | ggagaagtaa cccttactcc cttggtgact gctttcgtct caaagcacat gtgttcatta |
| 45901 | taaaatgtta atgtatttac accatagttg ctgtacattt aaaggtttac tgtgctttaa |
| 45961 | gctaaaacta gctggatatc aaatgtgctt ttaattgaaa agtggttatt tactgctttc |
| 46021 | cataggaatc cacaataaga ttttttcatt gcaaaccctc ctagtatctt taaaatgcaa |
| 46081 | ttcagttcac aaacattgat ttgcattcaa cgtttttcag aagcctaaat agagtgcaaa |
| 46141 | atgaggtcca cctgcaagac tacagttatt acttcctcct ttttcttgga agcatccatg |
| 46201 | cattgtaaat tctgtttgtg tggcatcagc caactacatt attaacatca ccaggaggaa |
| 46261 | ttaaaatgta tcatgttata ggatcaaaag tttcacattc tgaatcaatg ccagtataaa |
| 46321 | aagaatgtcc agacccttcc gtctgactac agtcggcagg gatccccgcc ccagcagcca |
| 46381 | tgtgactcac ttcgcattgt ggtgagggtc actcatggct catacgggcc tgcggcccga |
| 46441 | taggcctccc catggggcag catttagcct tttcaaaatc agcttaggat cacagaccct |
| 46501 | caggtcttga aggaactttt tggagtttcc tctattaaac ccagttttaa tctggtcatt |
| 46561 | tagactgcat gtctagaaac actacagcat ttttagtagt aaactgaaat aaatcttcaa |
| 46621 | ataaacgact ttaacatttt gcacattggt ccaggggcag aagagctcgt ctgttgcctt |
| 46681 | ttaggagggc tctaatcttt ttctgtttca gctcatcggg tcctcattgt cccctactgt |
| 46741 | atgtgcccag cacaatgtca gggcacattt ctacacaatg ttggattcca gctgatcctc |
| 46801 | cgtgttaatc cattcgttag tgccaaattg cagggttcct tgccaagccc gtcagcacaa |
| 46861 | tcagagcttc tgaagtcagg tacctcaatt acattagttc agttgtctca ttagccccca |
| 46921 | tgcatcatcc aaactcatca tcaaacgtgt tttccttcat tattgttgtc attttcttaa |
| 46981 | taccagtctt gttgaaaaag gattgtttta gatacaattt tgcctaactt gtttcatctc |
| 47041 | cagaatacaa tcctaaatag agccaagatt tgccggagag aggagatagc ggttgaggct |
| 47101 | ggtgaagctt ccctccggtt agaattagag agaagtggat gcagaacttg ggccagtaat |
| 47161 | ggacccatcc atctccctag tcaagaggac attcttgcag ccagaggtgg ttccgctttg |
| 47221 | gaaatgatct actttgtgta aaccggtgca tcacagatac tgttagagta tctcacagat |
| 47281 | actgatacag catggtctgc attttgtaga tatgagggct ctcccttagg tttacagccg |
| 47341 | gataaaggag ctccacttac ctggtgatca caggcatgtg gtccaccctg ctgggctcta |
| 47401 | ggactttgaa catagagaag gctatctttt aggaagactg caggatcagg agtcgggaag |
| 47461 | cagggattct gcttgtagtt ttacatctag cagtaacaac ttttagccca tccctcacct |
| 47521 | actctgtgcc ctggatttct tggctttaac atagcaaaag cctctcttta tagttttcct |
| 47581 | aactccatct ctccccaaaa agctggagat gtaagtttta aattccagtc cttggcacat |
| 47641 | agtaggtgct caatctctca tccttctgtc cctcaagata gccgatggct cagccatata |
| 47701 | tgttccagac aggtctctct gtgtcttttg ggaaagcctt gttgctctca gactggcctc |
| 47761 | taggcctcat gttttcccac ttgctcttgc cttcagtgtt aggtcatggg cttgcctcat |
| 47821 | tacctgtttc aaggaggcct ctatttgatc cctggagcat ccctgggagt ctgaggcctt |
| 47881 | taaggactcc tgtggtctca aggaaaaaga actataagta gttctcaggc tcccttgatc |
| 47941 | aattcaaagt catcttcagc tcttccagtg cagcccctcc agagctgttg aacgccacct |
| 48001 | ttttcctctt tcgggaaacc cacaaaacct tgcatggggt tgtgctctgt atggttttcc |
| 48061 | agcatcaaat tcactcatca aaagatgcat caggagggtg gagggggtgg cgcctggggt |
| 48121 | gagggtcaaa caaggaaggc cagaatgcct gtttgcattc gcaacgggaa cattggaagt |
| 48181 | tcgggtggaa aaacaatccc accggccttt atttaccaca gtgggtagcc tcgggaggag |
| 48241 | ggggagctgg gtgggaaagg aagacaatag aatgctgtgt tgccttgctg taaaagcttg |
| 48301 | ctctaagcaa ataaagtggc aagctcagtc agagccctgc tcaattagcc ccagtgctta |
| 48361 | aaggagggtc tccctgcagc tgttggcggc gggcgggctg gagcaaaggc aggcgcactc |
| 48421 | tggggcactc gggaggcgaa ccggcaggaa tcttgcatgg gagctgaccc gggagggaga |
| 48481 | ccaaaggacc ctccaacctg atcccagtcc ctgcttcttg aacagagggc tacagaaggg |
| 48541 | gttggtgggg ccactctggg gagaggcagt gtggggagac cacggaggag gtgaccagaa |
| 48601 | aagtgagtgc aaacgtttca tgccgagagt gaccgacata tggaacgtgt tatccggaag |
| 48661 | ggttacaggc tggaggagct cctagagttt ttcagggggt ggggtggggg agggggaagc |
| 48721 | cttcagaaac tcaaggaggt ttctaaacaa aaggaatttg agaagggttg gagaaataat |
| 48781 | gagaaatgca aacttgaccc agagtttctc cttccatctc acggcttctc ccctgcctgt |
| 48841 | cctcacatgt ttccagttca tggggaccca ggaaggccac tggagccctg tgcctgactc |
| 48901 | cacgtgcacc tcactggggg tgtgggtggc ggggtaggga ggacccgcag agctggctcg |
| 48961 | ctgctctgtg ctgaaaggga cccagagagc gagagccctg cctggcttta gacccctgtg |
| 49021 | gactagcagg ctgctagcgc cgggatggtc tctggatgat ttactccggt tcctgccctt |
| 49081 | gctggaggag caatttgatg ccggactggg agtgaaaaac agacttgccc agggactcac |
| 49141 | agtggccaaa ggggaggctg ggagtagacg caggctcttg atcccttctg ctttttggca |
| 49201 | tctccccttc tcagcatccc ttgcccagca caccacgcac acatgtacat acacacgcag |
| 49261 | acacacacac caaacagatg cacacagaat aacaacacca cacctacatg cacacataca |
| 49321 | cagacataca tgcagcacag acacacacag aataacacca cacatacatg cacaccacag |
| 49381 | atacacagaa taccacacac acatgcacac acacagacac attcacagaa tacatatatg |
| 49441 | cacacacaca catacataca tgcaccacac agaataacac cacacacaca tacagacaca |
| 49501 | cagacataca catacatgca cacatacaga tatactcaca gaatacacac atgcacacac |
| 49561 | atgtatagac atacaccaca caagcagaat aacaccacac gcacacacca cacacacaca |
| 49621 | cagaataaca ccacacacat atgcacacca tacagagaca cactcacacc tagacatata |
| 49681 | caccacactc atacagagac acatgcacac acatgcatac tacacacatg cacatgcaca |
| 49741 | cacaaaaaca tacacagaca tcacacatgc atacatatac acaacacaca gacacaccca |
| 49801 | cgggcttaca cagacaccac acacacgtag atatgcacac agcacacaca cacacagaga |
| 49861 | aacagacaca ctcgcaggca tacatgcatg catacacaca ctacacacag acacactcgc |
| 49921 | aggcatacat gcatgcatac acacactaca cacagacaca tctgcagtca tacacaggca |
| 49981 | ctacatacat agagacatac aatgcacact cacacacaga cacacataga cacatgttgt |
| 50041 | gtgctcatgc gtacacgagc gcgcacacac acacactgcc tgacttgttt cccacaagac |
| 50101 | gggtactggc ctgtcgcttg tagccctcct gtcccagcct gtgttggcca gggtgccagg |
| 50161 | cactgccacc cctcttggga caaggtacag gtggccagtg tgatcacggc ctgttctgag |
| 50221 | agcctcttcc tgagccagga agcgctgtgt gatactgagt gcccgtgcct tcgtctttcc |
| 50281 | catggtgctg ggtcttggcc acctgctgca ttgatagcac ccgcatgttc acttccctgg |
| 50341 | cagtagaaag aaatgtaggt tagtgcaggg aggtcactgg ctttggagat ggtggcaggg |
| 50401 | tggagcagtc tctaatgtga atggaagtgc acatgccctc tggacctgca gcaatgctgg |
| 50461 | cttcagaggg gccttctctg gtacctttcc aattttcccc aaccaggtag aagagccact |
| 50521 | gcccagtgtc ttgggctcaa ctgaaacccc ctgtacaaga aagagacccc cctttccact |
| 50581 | gtgtctctct cctcctcccc actcactgtc tctctgtctc cctcactccg tgtctctctc |
| 50641 | ctctcttctg tgtctctctg cctctccctc tctctgtttc tctctgtatc gctctttttt |
| 50701 | ttcaatctct gtctctatct ctcctccttt ctctttatct ctctgtctct ccctctgtct |
| 50761 | ttctctctgt gtgtgttttt ttttctgtct ttctgcatct ctgtttctgt ctccttctct |
| 50821 | cggttctgta tctttgtccc cctctctccc tatctctgtt tctctgtctc agtccctttc |
| 50881 | tgtctttatg tctctgttta tctctctcag tctctgtatc tctgtctctt tctcttttta |
| 50941 | tcctttctct gtctctttgt tctctatccc tccatctctc tctccctctc cctcagtctc |
| 51001 | tctctgtctc ttcctttctc tgtctcagcc tctctctgtc tctgtatctc tatctctttc |
| 51061 | tctctctccc agtctctctc tttctatctc tctctctctc tctctctctg gcactcactc |
| 51121 | acccacttac ttgaagtctc catgagcagt gggtggcttc acctttctgt ttcgccactt |
| 51181 | tgcagtccga cccgtggggc ttgcagaccc tcttctggcg cacaccttca ggagaccaac |
| 51241 | ggtgccaggg cactcccgtg ttcttcaggg ttccagcccc gagtagttgg taaacatcag |
| 51301 | taatcgtcct agagatccac tgtagattcc tcatccaggt actgaatgag gcccttctga |
| 51361 | gcaaatttaa tggaatgacc ttggtgacat tacaagatga cggctcatct cctgtagtct |
| 51421 | attattgttg ggcatttagg ttgatactat gtcttcacta ttgtgagtag tgctgcagtg |
| 51481 | aacatatatg tgcatatgtg tctttatgaa agaatgattt atagtccttt gggtatatac |
| 51541 | ccagtaatgg gattgctggg ttgagtggta tttctatttt taggtctttg aggaattgct |
| 51601 | taggtctttg aggaatcgca tttgcgtttt caaacatcat ggagaacaca ggtcttgagg |
| 51661 | atgtgcagga acatggagac caaggaatga aaaagccatc aggttggaag tgaagtcacc |
| 51721 | cagagtgatt gcaggtttat ggacagaaag gaaaatgctc tgattcctgg tgtccatgag |
| 51781 | gaaggtggat gccaatagat ggtgctacac agacacttgt gaaaggaaga agggaaagag |
| 51841 | tctgcctcca tgggctgagt agggctgctg gggcctcagg cttcacacat agtgtcagac |
| 51901 | atgctgtgtt ggtcaggttc atctgggacc cctgtgcctc ccccaggaaa ccctattggt |
| 51961 | aggtgtgggc aggcttccct ccatgtgtcc ttaaatacac tgactgctct gtgtgtgact |
| 52021 | gtggatgagc agcgtagggc tggtcatctg gagggctggg cttggttcca gctcttgtac |
| 52081 | tgatggattg attggttttg ggcaagtcac ttccccccat aacctttcca ttgtaccaca |
| 52141 | atcctaatag agttggatgc cagtttctgg caccctgggc ttcacaggga cacagagaaa |
| 52201 | cagagaggac cagaaaatta ggctgataat aatcatttct tctctatttg gtctggagaa |
| 52261 | gaaatgactt agggttggca caggttaata tgtcaactag aaagaacctt tgaaaatctt |
| 52321 | tagttcaaga attctaaacc taaaatctgt gggacactag gatgattttt atggactctt |
| 52381 | tgaatcaccc tgaaattttt taaaatattt tttctttatg ggcacatgtg tggtttttta |
| 52441 | gcagaaggat tccttagcat tgattacatt gtaaaagggc caacaatcat aaaacaatta |
| 52501 | attccccagc atcatgttat gtgattcata actgaggggt ctccgcagat ggcatttgga |
| 52561 | aacgtgcggg tccttttttt cagttgtcag catgactggg gtcacgatgg atatttagta |
| 52621 | ggtgggggcc tgcgttgcat gcgtggggga ttaaacttaa caataaaaaa ctgtcctgtt |
| 52681 | tgaaatccca gtagcacccc tctggagaaa cactgctcag gaactgagcc cccaagatgc |
| 52741 | acagtgattc tcccgtggcc acaaagctca ttagtggcag agttggtttt tattaacaga |
| 52801 | tcaaaacagg ggatgtgcca agaacctaca ttattttgtt tctcatcatc agctgcaaat |
| 52861 | gtgctgcaat ctgtgaaaac aaaagaatca gaaatctgtc ccaccttcaa cgctccagcc |
| 52921 | atttttaaaa atgagtcttt gggttcttgg ttgtttttta ccatcaaatg agaaatgagg |
| 52981 | aaaggaatat tttacatttg gagaaactaa accataaaca attgatccca ctagccccat |
| 53041 | atcacctaga tgtgttcttc agctactgtg aattggtgac gcaagcctta gactggaaat |
| 53101 | ttccccttta tgtttcagag gatctgtctt gaatgtctct tactctacaa agaaagaaac |
| 53161 | attaatatac cagcatgctg cttgctacct agtttactgc aatgaagtgg caggtgcctt |
| 53221 | agactttgga gtgaaattga gagatattcc gcagtattag ctaagagtta ggcctgtggg |
| 53281 | atcacagaga caggggtttg ttccctggct ctgtctctca attgatgtgt cttcttaaga |
| 53341 | aaaagtactt aatccttctg agcctcagtt tccttggctg aaaagtgggg ataataatcg |
| 53401 | tatccacaaa gattaattga gatcatccat gtgaagtgtt ccgcacagtc tagcatatgg |
| 53461 | ggaggctcaa taaatgtgag ctgttattac aagcgattat tatgacttgt gtctgtgatt |
| 53521 | aaagacagcc tgaggttcag agtgtttgtg ccaaaattgc tttcagagga caagctctgg |
| 53581 | gttggttttt cagtcttgcc cagctgccca gagtcactag agtgtttggg gctggagtca |
| 53641 | cagtgtttat taagtgccta ccacacactc cagttttgat ggaagattgg ttttcttcct |
| 53701 | gtgtgcacat ccccgcatca ctacatgagt gggtgagaag agactcagaa aagcaccaga |
| 53761 | cacatgtttc ttctgcctca gttccctagt etgtaaaagt actcaagagc atgatggegg |
| 53821 | ccacagggaa cagaggggag agacacctgg aggagcaaaa gacagtccca getgteagte |
| 53881 | ttcagacacc atggttttct ggagaaggga tccagaccag accgccaatc aatatageeg |
| 53941 | cagaccgcca gtcaatatac ctcatccttg tgaaaggegg ttgtctgtgt ggcagcaggg |
| 54001 | agacaggagg ggccatattt gacacaagcc tgctggccaa gtetaaagga gtcagcctgc |
| 54061 | catctgacca cactttctgc agccaagtcc tctgggggcc aaatcagtat ggtttgagtt |
| 54121 | tatccccgac ttgctgctat ggttgatgca cacaaaaaag gtttggccat tgccaagcca |
| 54181 | gctgtggtct tggtttggaa gaggcaacca aagtaagaat tgaagggaag gcatgctttg |
| 54241 | ggatttccct tagccttcaa ccctctagag gaagccaact tetttattet gccattttga |
| 54301 | gactcatgct tgtctgggtg gataagataa atactttcat cattatcatc attattatta |
| 54361 | attataatta ttgaaaatca attactcage agcccctacc ttgtaatggt atcacttaca |
| 54421 | gttgtatagc attttgcaga caacgtatac actagaccat aaactcagct cgaacataag |
| 54481 | cataaattta aaacaaaaac attgaettgg geagetatta tetgaggtea aagttattet |
| 54541 | ttaattccag ctgtaccttc agetgeetat gtcgctgaag caacttcccc ttgtgtgttt |
| 54601 | tcacacttag agattggagt aatccagaca cagaaaatga taccaaaatt gaaatgtttc |
| 54661 | tgaacgttaa atgattcaat aatttgaaaa tagtttcaaa tttttcaaaa ttcaaaaatt |
| 54721 | tgaaaatagt tcgactagag cccatcctca cctcaggtag ttacgtggtg tgtgtgtgtg |
| 54781 | tgtgtgtgtg tgtgtgtgtg tgtgtgtagg cacacaaacc eatgeatgea cacatgtatg |
| 54841 | cacagtggtg gatgagtgtg agttaccaaa acaaatacca ctaaatgcag gataacacca |
| 54901 | tgatggctag tagctggatt gactcagatt ttctctgcag atccttccta gtccaccctc |
| 54961 | cttccatgac atgggagttt gtgatatgea aaggataaag taaacctgag tttcctgggg |
| 55021 | cttgtgtcgg cataccaagc teetttaaga tctctctttg ccaagatggg gctaagccag |
| 55081 | aggcttcctt ggcctgggga ataactgtcc taccttcctc cctggagcgg ctggcgcaca |
| 55141 | ctgttttgac gtgtgccatg ccttccatct tttacggcct cggggccaga ggagggtatg |
| 55201 | ctttgaacaa tgtgaaattc ctctcctaac agetgtgeaa aggaaactca cagctttcta |
| 55261 | tctccatctc ggtccactta gctttctttt ggggtatagg tttcttgtct gttcaggggt |
| 55321 | ccctgttcct cactttctgg cettcagaag gaccagtttc agtacttcct ttcttaggga |
| 55381 | ggcaagtcag tcttctgagg ttetetgttt tattttattt tatcctagag tatgeettea |
| 55441 | ctgttgaccg tgtcttgcct catttctgct aagaagccag eatettetga aaattgagtg |
| 55501 | tcttgtcctg tttcagatct tgggccgagg attgaccatg tatgttagtc agtttcagag |
| 55561 | ccctgagttt taagtgctaa gttttaagga gaggaacaac agtagaatta gatatettaa |
| 55621 | agaacaggat aggaatccac ttccetgccc cttctctcac ctggtttgac actacataga |
| 55681 | tacagacctg tcctgagttc agggattgtc taatgaggee tcaccaaata ccaacagaga |
| 55741 | gactagctgt ttctcctcta gctcactgac tgettetagt tttccgtgac tttagagcat |
| 55801 | gtgcgtcagc aggttttggg gaccatctct ggcctgcttt etgatettgg ctctgtccct |
| 55861 | caatttatgt cttttttgtg ctccataaga cagggagttt catggttgcc actacccgag |
| 55921 | aggtgtgcta aagtgaatga ggtggaggac acagagctag cagggaagaa ggtgccgtgg |
| 55981 | aaacccacac agctttattc atgtgagctc atttctcatt tactccttgt agcaatgcca |
| 56041 | agtcaccccc tcccttcatc cctttccaag catttccctc ccttgttggt tttggatcca |
| 56101 | ttgtaacccc agtgagttaa cctacatgtt ttagtttgaa tctaatcatg ctatttccag |
| 56161 | gttgccaacc cccacgggtg cttcagccta attggtctgt cctcactggt gtetgeagea |
| 56221 | ccttcccttt gagcgtcatc tgtgcatttt aattaatctg ccactttttc ctccctcctg |
| 56281 | cttccggatc attaataaag agattaacca aaaccagacc aatccccagc ggaggctcca |
| 56341 | ccaggatctc tctctcccag ctctcagctt tgtcatgggt cagaacatca tcatgtggcc |
| 56401 | ecattgacta attctgaggc ctcaagacct atgttcagag caaagccagt gtgaattaat |
| 56461 | ttttcaatta agatttgggg aacaagcata gccagtcttt tatatatggc cttagtttaa |
| 56521 | tatttccacc acattccttt cccttttgta attetaettt aaatetatea ctttcccacc |
| 56581 | tctctccctt tccagtccct gaacaagaca ttagggttat ctggcaggat gtctcctgag |
| 56641 | cctgtagcat tagteagaga teatgteata geegaagaaa ttctccaaag aatcaggccc |
| 56701 | ttctggacac agctggccag cgtcctcttt gaccccagcc agagaatgea tttgcctcca |
| 56761 | gagaatatgg ctacaccaga aataatgaaa tetettgaat ttgeatatta aaaatttggc |
| 56821 | aaattaaaat ttgctttcac ctctgctctc atgactcctc tetgaggaaa gctaaaacag |
| 56881 | agcctttctg ctttctttgt aagtcaccca gatttccagc getggtetea aacccaggat |
| 56941 | gtgagatggt gcactccgtg ttctgtccac tacaacacag ggeetetgea ggcacaggtg |
| 57001 | acctgcagat gggctgcact ggagagcatc aggcccagga gggactaggg tgagggaatt |
| 57061 | ggattgtgtt tacttttgtg ggggatgctt tcatagggaa tacagggagg aggacacagg |
| 57121 | attgatggcc tcaaatctct gcaaactctg cttagagctg aggcttcttg gggaaggaca |
| 57181 | cagcctgctg acagcagccg tcctggctcc aggcttggag cttgggcttc ccctggaagt |
| 57241 | aaactctcta gaatgtttga gagcaaggac agacggagag gcagccgagt gtcatcatta |
| 57301 | ataacatgag ctctagagcc agaaggtgca aatcctgact ctgttgcttg ttagctggtt |
| 57361 | gtggaacccc tctttgcctt ggtttcccca ttcaaaatgg gaaatataat agtacgtact |
| 57421 | tcaggatggt tgttactagg atttaagatt tatgtaagcc agtacctggc actttgcatt |
| 57481 | tttattcttg gtgatctcac ccctaagcag tctaaaaatt tacttcacac atgtgcagca |
| 57541 | agaggcgtct ttaggtacac ggtcaggagg tgctcctgtg tagggaagct ggtcctggag |
| 57601 | aggtgaattg gaacctgaag gctgcagccc agtctctgca cgtgacaggg cagacgcagg |
| 57661 | ttgctggggg ctcagattct gtgccactgt ctacctcagc ttccttttcc atgtcagctg |
| 57721 | cttcttgaat acccccaaac cctctgtgga tgctgtaaac tgagccaaaa aggaggaatc |
| 57781 | agaaattgct ctcttttgag tattttgagg gatggtgaaa ccttgtagga atgttgctcc |
| 57841 | agggactgag aaaacaccat atgtgggaaa gcgtttctaa gtgaggaaaa tcgcacatgc |
| 57901 | acgtcggtca ggatcttgga aaactctgag caggggaaat gggcactggg gagagtgtag |
| 57961 | aaagggggtt cactaaggtc tttaacaggt ctttttctaa ttatagccaa gaagagcctg |
| 58021 | gggaccatgg ccgacttcag agccagggcc tgtattttct ttgggaaaag aaggcagagt |
| 58081 | tgattggctt ccaaaaccag gcttggcaaa agcccatgca ggctctccag ccagtaagca |
| 58141 | gtgcctgggg taggtctcga tcgtagcagg cgctcattca gtaaatagac gtcgaaagtc |
| 58201 | cgcagcgtgt ccagccctat gctgggagtc aaggatgtgt ggatggaaag gacatggtcc |
| 58261 | ctgtcgtcaa agagaaggca caccaacaat aatagcacaa tatgacaatt gcttaaatga |
| 58321 | agctctctgc agatgacagg gacccctaga gaaagagcgt agacattccc ctaaatagca |
| 58381 | gaactcgctg ctgtgccttg cctggatgga gctggcactg ccttcctgtt tccattcccc |
| 58441 | actccaggga actggcccac tgggggactc ccaccccgga catggagtga gaaatgaagg |
| 58501 | tattggtggc caaaaagtga ccagagatca gaatgcatct attcttgccc ccagcaaatt |
| 58561 | cagaaacacc acctctccct tgcccgtgtt tgctttcgtc atttgtctgt tctctgggct |
| 58621 | tcgagttcat aatattccta tcttctccca caaccagtgt aactggtctg agatccaagc |
| 58681 | agtcgggcac catggcatct gggggaaagc cttgtgggga ccgtgttggt aaagattgct |
| 58741 | gtgagccaga tgtagaggag ggagctctcc agactctggg tccctcgccc gtgtgcgtca |
| 58801 | agggcaggtg tgcaccatct cccgtggcca caggccattc agcttcatgt tctctaacat |
| 58861 | ttctagtgtg ctcactctgt acctgatgct atgctaggtg cttccatgtg gatttcattg |
| 58921 | tttacccctc acgataactt tcatggtgca caagccatgt gatttttgtt gcgcctacga |
| 58981 | accaaatgcc cccgtcactc tctgccattg tttagcagtc tgaccttaga cagctcactt |
| 59041 | caccacaact gcaggcctcc gttcccttat atgtataaat gaggaggttg tctagggtcc |
| 59101 | tttccagctg caaacctctg tgttttgggg agttctgtta gcaacattct tggacttgct |
| 59161 | ctctacgaga aaagctagaa gtcgattatt aaaatggagt ccacaggaag cctgcagggc |
| 59221 | tttaagacag ccttctaaag agtttagaaa cactcaccag cctgcagatg acttactccc |
| 59281 | actacttatt acaaagtatc atggcaagta agaaagaaat ggtcagccac tgggagagca |
| 59341 | ggctgaggtt gcaaggaagt ggcaggtgct atcgttggga tcttggggga gcattgtaga |
| 59401 | aaggtcagct ttggaaccag gcactgaaaa acgggtgaaa tttcaacagg agaggcagtg |
| 59461 | cacatgggtt gtgatttgag cagcagtgag tagtgaatgg ggttggagca agaggcctgt |
| 59521 | gagcccagtc atcacatgga aaagggaggt gagggccaga gtgcagagaa cccctctcac |
| 59581 | tttcctgtgg gtctctcctc ttcttttttt tttttttttt ttttgaattg gagtcttgct |
| 59641 | ctgtcgccca ggctggagtg cagtggcgtg atgttggctc actgtaagct ccgcctcccg |
| 59701 | ggttcacacc attctcctgc ctcagcctcc cgagtagctg ggactacagg ctgccaccgt |
| 59761 | gccctgctaa tttttgtatt tttttttaga cggggtttca ccttgttagc caggatggtc |
| 59821 | tcgatctcct gacctcgtga tccacccacc tcggcctccc aaagtgctgg gattacaggc |
| 59881 | ataagccacc gtacccggcc tttctcctct tctttaaact gcatttaggt tgtcctcatc |
| 59941 | tataagatga aggaataaga ttagatggcc tctttatatg gcttatctac ttctaataaa |
| 60001 | ctttggttcc atattccagc caccacccat aaagactctt actttttcct cctcaatcca |
| 60061 | tcagcatcag ccagcattct ccctctcagt tcatcctggt cattacctca ctaatctgta |
| 60121 | ccttgactct tattctacct gggacctccc ctcactaacc atacttgatt tatttcctta |
| 60181 | agageggata tcctgtgtat tcttagctta cagtggctga gcttttggga ggtttaacaa |
| 60241 | attgtttttc aatagactct cagtcctaaa tgattttatt gcagctgtat tattcttttt |
| 60301 | gaggggagta gagaatgtaa tcctagttgt atgccatatt ttttgtattg ttcaagttgc |
| 60361 | attttgttgg gcctgtaata ttgaagaaaa tgtcacttgt atgcagaata ggagggagat |
| 60421 | tccttatgct gtagaggaac cattttccta ggatagtaca attccttaga gtcatctcta |
| 60481 | agggtgagca gcaaagctag aatccctctc ttggctttga ccttgaagga gtcagttttc |
| 60541 | ccaaagacta gttcccatgg aaagaagatg gtctccttat cacagcagca agaggaggca |
| 60601 | gtatgagcag agtgcctcac tgttgttgac ctctagggac aagtgagccg gcagtatttc |
| 60661 | agacaagctg ggaaagaggc tgtctgtgag tcctgggagc gagtgagcac tggcaggctc |
| 60721 | agacataggt gctggttagc aggactgctt ttctgtttct tgtgtcggct ttgtttattt |
| 60781 | cctctctatt ttccccctgg acttagtaaa gtctttccga aaataccaaa ggtgaaccag |
| 60841 | gggaagagtt tttattttcc atgtttggac agaactttaa agaggaaatg atgtaccccc |
| 60901 | ctgggagcca gtgaggtggc agcgatggtg attaaggagt gaatatctca aggaggtgga |
| 60961 | cgaattcggg gatcactagc tcagctgccc ctctccacct ggagcatctc cttccagtgc |
| 61021 | taccctcaga acatctgggc tttgctctag tgagggagag actagcaatg aaggtgtctt |
| 61081 | gagatcagca ctgtaattcc accaggacgc caccggagtc cggtgttaag cttctactat |
| 61141 | ggcaacagaa tgagagcgtg gatgggttga aatgccattt caacaaggaa atagtagaat |
| 61201 | tagaagaagc ataagcacta aaaggaacat tttgtagcag aaatgttaaa aatctaaagg |
| 61261 | aggcaagtga atcaacaatg actcttctat tctgggcaag tccagcccat ttatgtaagg |
| 61321 | tggttattct gcatctctgt cttctgcaag tagtgctgtg gcagagctgc gttttgtgga |
| 61381 | gagcgtcccc ggggatggag cagatcagtt ggtgatgcgt atgtatcaga aagctcggca |
| 61441 | gagcaccctg gaacgtaggc cctctcgcgg agtgggtagt ggccctacat gttcatttcc |
| 61501 | aagggcagga gaatagaccg ttccagctgc ggcctggcca gggatgaccc cacatctgac |
| 61561 | actgcaatat gggggcaact gaaccagtcc tcagcctcag tgtgttccag gggctgcagc |
| 61621 | tggggagcag tcgaactctt tcttgagaca attacaaggc caccgctgct gctgctgaag |
| 61681 | ggaagttact ccatgtttac aattctcagg tttgaagttt tcatgctttg ccaaggtaga |
| 61741 | gtgaaccatg cgtctttgca ggctcaaggg atgtttaaag aagcggtagg acatcgtcca |
| 61801 | cccacaagca gagaccgcag gataaagcag acatccaatg taaatacaac ccgtgcaaaa |
| 61861 | agcagagtcg gcagacctgg agtgcattcg cagtatctcc cgggggtggg ggaaagaaat |
| 61921 | cacctcttca gaatgtccag aggggagttg ccttgcttac ctggggggcg gtaccctctc |
| 61981 | tcgtgccctc acagggctac tcagcctcag gtagctggtg ccagaataac acagactcag |
| 62041 | ctgccagagc ctgctcttaa cacctgtgtt tccttttcag atcttacagg tgaacaaggt |
| 62101 | gatgtccatc ttgttttatg tgatatttct cgcttatctc cgtggcatcc aaggtaacaa |
| 62161 | catggatcaa aggagtttgc cagaagactc gctcaattcc ctcattatta agctgatcca |
| 62221 | ggcagatatt ttgaaaaaca agctctccaa gcagatggtg gacgttaagg aaaattacca |
| 62281 | gagcaccctg cccaaagctg aggctccccg agagccggag cggggagggc ccgccaagtc |
| 62341 | agcattccag ccggtgattg caatggacac cgaactgctg cgacaacaga gacgctacaa |
| 62401 | ctcaccgcgg gtcctgctga gcgacagcac ccccttggag cccccgccct tgtatctcat |
| 62461 | ggaggattac gtgggcagcc ccgtggtggc gaacagaaca tcacggcgga aacggtacgc |
| 62521 | ggagcataag agtcaccgag gggagtactc ggtatgtgac agtgagagtc tgtgggtgac |
| 62581 | cgacaagtca tcggccatcg acattcgggg acaccaggtc acggtgctgg gggagatcaa |
| 62641 | aacgggcaac tctcccgtca aacaatattt ttatgaaacg cgatgtaagg aagccaggcc |
| 62701 | ggtcaaaaac ggttgcaggg gtattgatga taaacactgg aactctcagt gcaaaacatc |
| 62761 | ccaaacctac gtccgagcac tgacttcaga gaacaataaa ctcgtgggct ggcggtggat |
| 62821 | acggatagac acgtcctgtg tgtgtgcctt gtcgagaaaa atcggaagaa catgaattgg |
| 62881 | catctctccc catatataaa ttattacttt aaattatatg atatgcatgt agcatataaa |
| 62941 | tgtttatatt gtttttatat attataagtt gacctttatt tattaaactt cagcaaccct |
| 63001 | acagtatata agcttttttc tcaataaaat cagtgtgctt gccttccctc aggcctctcc |
| 63061 | catctgttaa aacttgtttt gtgatccggc tctcaggagt cactctgtaa aatctgtgta |
| 63121 | caccagtatt ttgcattcag tattgtcaag gccatgactg ttgttttagt aaacttgtta |
| 63181 | aaatca |

Brain-Derived Neurotrophic Factor (BDNF)

The BDNF gene encodes the brain-derived neurotrophic factor protein. BDNF is expressed only in inner hair cells and outer hair cells during the neonatal stage. BDNF supports connectivity to SGN. BDNF induces synapse regeneration and SGN protection after damage (Takada et al. (2014) Hear Res 309:124-135; Budenz et al. (2015) Sci Rep. 5:8619).

The human BDNF gene is located on chromosome 11p14. It contains 2 exons encompassing ˜67 kilobases (kb) (NCBI Accession No. NG_011794.1). The full-length wildtype BDNF protein expressed from the human BDNF gene is 255 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype BDNF protein is or includes the sequence of SEQ ID NO: 31. Non-limiting examples of a nucleic acid encoding a wildtype BDNF protein is or includes SEQ ID NO: 34. As can be appreciated in the art, at least some or all of the codons in SEQ ID NO: 34 can be codon-optimized to allow for optimal expression in a non-human primate.

Human Full-length Wildtype BDNF Protein
(SEQ ID NO: 31)
MFHQVRRVMTILFLTMVISYFGCMKAAPMKEANIRGQGGLAYPGVRTHGT
LESVNGPKAGSRGLTSLADTFEHVIEELLDEDQKVRPNEENNKDADLYTS
RVMLSSQVPLEPPLLFLLEEYKNYLDAANMSMRVRRHSDPARRGELSVCD
SISEWVTAADKKTAVDMSGGTVTVLEKVPVSKGQLKQYFYETKCNPMGYT
KEGCRGIDKRHWNSQCRTTQSYVRALTMDSKKRIGWRFIRIDTSCVCTLT
IKRGR
Mouse Full-length Wildtype BDNF Protein
(SEQ ID NO: 32)
MTILFLTMVISYFGCMKAAPMKEVNVHGQGNLAYPGVRTHGTLESVNGPR
AGSRGLTTTSLADTFEHVIEELLDEDQKVRPNEENHKDADLYTSRVMLSS
QVPLEPPLLFLLEEYKNYLDAANMSMRVRRHSDPARRGELSVCDSISEWV
TAADKKTAVDMSGGTVTVLEKVPVSKGQLKQYFYETKCNPMGYTKEGCRG
IDKRHWNSQCRTTQSYVRALTMDSKKRIGWRFIRIDTSCVCTLTIKRGR
Rat Full-length Wildtype BDNF Protein
(SEQ ID NO: 33)
MTILFLTMVISYFGCMKAAPMKEANVHGQGNLAYPAVRTHGTLESVNGPR
AGSRGLTTTSLADTFEHVIEELLDEDQKVRPNEENHKDADLYTSRVMLSS
QVPLEPPLLFLLEEYKNYLDAANMSMRVRRHSDPARRGELSVCDSISEWV
TAADKKTAVDMSGGTVTVLEKVPVSKGQLKQYFYETKCNPMGYTKEGCRG
IDKRHWNSQCRTTQSYVRALTMDSKKRIGWRFIRIDTSCVCTLTIKRGR
Human Wildtype BDNF cDNA
(SEQ ID NO: 34)
atgaccatccttttccttactatggttatttcatactttggttgcatgaa
ggctgcccccatgaaagaagcaaacatccgaggacaaggtggcttggcct
acccaggtgtgcggacccatgggactctggagagcgtgaatgggcccaag
gcaggttcaagaggcttgacatcattggctgacactttcgaacacgtgat
agaagagctgttggatgaggaccagaaagttcggcccaatgaagaaaaca
ataaggacgcagacttgtacacgtccagggtgatgctcagtagtcaagtg
cctttggagcctcctcttctctttctgctggaggaatacaaaaattacct
agatgctgcaaacatgtccatgagggtccggcgccactctgaccctgccc
gccgaggggagctgagcgtgtgtgacagtattagtgagtgggtaacggcg
gcagacaaaaagactgcagtggacatgtcgggcgggacggtcacagtcct
tgaaaaggtccctgtatcaaaaggccaactgaagcaatacttctacgaga
ccaagtgcaatcccatgggttacacaaaagaaggctgcaggggcatagac
aaaaggcattggaactcccagtgccgaactacccagtcgtacgtgcgggc
ccttaccatggatagcaaaaagagaattggctggcgattcataaggatag
acacttcttgtgtatgtacattgaccattaaaaggggaagatag

A non-limiting example of a human wildtype BDNF genomic DNA sequence is SEQ ID NO: 35. The exons in SEQ ID NO: 35 are: nucleotide positions 1-647 (exon 1) and nucleotide positions 63474-64238 (exon 2). The intron in SEQ ID NO: 35 is: nucleotide positions 648-63473 (intron 1).

| Human Wildtype BDNF Gene | |
|---|---|
| (SEQ ID NO: 35) | |
| 1 | attattaaag cggtagtctg ccggcgctga taagcaacaa gttccccagc ggtcttcccg |
| 61 | ccctagcctg acaaggcgaa ggttttctta cctggcgaca gggaaatctc ccgagccgaa |
| 121 | ttcagcttcg ccggagcccc aggtgtgacc tgcgtagtgg gcaagggagc ggtgtgcagg |
| 181 | ctgagttttt ttttttacag gggtaccctg aaactcctca ctttctctgg gaactttcag |
| 241 | tgccaggacc cagtaacggg cggttagaag gcagccctag gaaacacctg ctacatagca |
| 301 | gggcagttgg gcaatcattg gtaacctcgc tcattcatta gaatcacgta agaactcaaa |
| 361 | aggaaacgtg tctctcggag tgagggcgtt tgcgtaaatc tataggtttt tcgacatcga |
| 421 | tgccagttgc tttgtcttct gtagtcgcca aggtggttga gagtttaagc ttgcggatat |
| 481 | tgcaaagggt tattagattc ataagtcaca ccaagtggtg ggcgatccac tgagcaaagc |
| 541 | cgaacttctc acatgatgac ttcaaacaag acacattacc ttccagcatc tgttggggag |
| 601 | acgagatttt aagacacttg agtctccagg acagcaaagg cacaatggtg agtagcaata |
| 661 | aaacctgcat tataattgaa aaatcttgac atgttgctta acaacgggca tatcacggct |
| 721 | cttcctagca cttcacacgc caaagaacag cagctactca ggccagggga atcgggtttt |
| 781 | tacacagtgc aactttaatt ggaatcattt gagatttgac acagctatgt ggaactgcgt |
| 841 | ggaacaaact tggagctggg tgggggggtg tgtgttatat tggttgttca aggctgatgc |
| 901 | ttgtctctca gcagtcttgc attctattct tttccttaat gtgtatggtg tatgatcata |
| 961 | ttctatgatt tatatgtggg catgtaattg acatttgcaa gggggttaat ttccatctaa |
| 1021 | aaacaataat gctgttagag gttggggtta gggggtggag tgggggtaag ggtggggtaa |
| 1081 | agactgggag tttaggtgta gatggggggt ggggttgggg ggagagaaat aagtcagaag |
| 1141 | tgcatatcac cggtaatggg taatcctctc gtagaagaaa aggttctcat caacatgtga |
| 1201 | tcaactatta acaggatggc tttggcaaag ccatccgcac gtgacaaacc gtaaggaagt |
| 1261 | ggaagaaacc gtctagagca atatcaagta tcacttaatt agagattttt aagccttttc |
| 1321 | ctcctgctgt gccgggtgtg taatccgggc gataggagtc cattcagcac cttggacaga |
| 1381 | gccaacggat ttgtccgagg tggcggtacc cccaggtagt cttcttggcc ccgctgtaaa |
| 1441 | gccaaccctg tgtcgccctt aaaaagcgtc ttttctgagg ttcggctcac actgagatcg |
| 1501 | gggctggaga gagagtcaga ttttggagcg gagcgtttgg aaagcgagcc ccagtttggt |
| 1561 | cccctcattg agctcgctga agttggcttc ctagcggtgt aggctggaat agactcttgg |
| 1621 | caagctccgg gttggtatac tgggttaact ttgggaaatg caagtgttta tctccaggat |
| 1681 | ctagccaccg gggtggtgta agccgcaaag aaggtaagca ccagggcggg gaccccttgc |
| 1741 | atccccaatt cttgagctat tttgatactg tcttccggag aggacgcgtg gtggagggga |
| 1801 | ggaggtagag ggagagcatg agagggggtt gtttcttggt atttgcccag tttgaattgc |
| 1861 | cctaggtgag aaccctgggg caaagggaga aagaaaaaaa agaaactcag tcttcctgcg |
| 1921 | gatataatga gtttagttaa cttggacctg caaatgtctg attcaaatgt aagatttatc |
| 1981 | tctctttttc tcctcttcac ctccctcttt tccgttctct ttgctggtgt gtgtgtgtgt |
| 2041 | gtgtacagta gattcattac taattatgaa gcttttgcaa aacattcgaa ttcctaaaat |
| 2101 | ttgactttgt agcatttaga atcaggcggt ggaggtggtg tgcggtgggg agaggaggtg |
| 2161 | gaggttggga agagggaagg aggtaaagct aaacctccaa cacaaaaaaa tgaatcaagg |
| 2221 | taatttcagc tcttctagtg agaaggattc attctctctg tatccctccc tccctctctt |
| 2281 | tccccctccc tccctccttc ccgcccccct tcttccaccc cgccccctcc tccagcctcc |
| 2341 | atccctccct cattctatct cttcctctcc gtcgccctcg ctcctcgctg gatgcttctt |
| 2401 | tctgggtttt cttttttttt tcccttctgt cctccctccc cgcgagtttc gggcgctggc |
| 2461 | ttagagggtt cccgctttct caagggaagg ggagctgccg agaccgcgct ccgctcccca |
| 2521 | gccgggccgg atgcctcact gagcccaggt ccgagtcagt cggggtaact cagggaaagg |
| 2581 | ggagcctccg cctgggagta gaaggtcctt tccggaccga agagccagag agcgggccgg |
| 2641 | gcgagggggc ctgggcggct ggaggcggtg gagaagaaca cttttagctc cgtgcggcgg |
| 2701 | ctggacagag ccaccaatca gctggacgcg cagaccgccc tgccagggcg aggttgcgtc |
| 2761 | cggaggcgcc ggtggagggc ggccggctag tcgctgagcc gccgccgcca cccgggtggg |
| 2821 | caggggactg gcggtgggtg gaggtgaggg gcttggcggg tgagatagaa gcggcgcgga |
| 2881 | gccgcccaga cctgtgttct acctctcccg cccccgcctg cacccccggg ggacagcgaa |
| 2941 | ctgccggaac gcgcggctgc gttatcctct tgccactctt cagggagctc agggacttag |
| 3001 | gcgcccctgg gcgggggcca ccaggctctc cacactccta taaccctcac ccccaccccc |
| 3061 | ttctcaggcc ttttgttccg gccacagagc caagcccggt ggcagttttc gccccagggt |
| 3181 | gtaggggtga ggggaacgta ggaaaaatct gtttccgaaa ctcaagacca ctgttttaac |
| 3241 | gaacgaaaga aagaatccca actctgcgca ggtggattca taggcgaagc gaggatattg |
| 3301 | tggaaattca gaaggaaaag ataaaaaaca ggcgctagga tcagatgacg gtgataggct |
| 3361 | gctcggcaca caaagggagc gtagggcagg gtttacggag caagcctgca gcgaatgggg |
| 3421 | cacagattgt tccgagatcc agtcgttttc tcagtcagat ctacgcgaag ggaggggagg |
| 3481 | ggaggggcgg gcaggggagc gtggcgggag gggctgagct tgggggcggg gggatttctg |
| 3541 | atcagtctga tgcaattcca agcgtgctgc aaaggaactc caaggcgccc gcatcaccat |
| 3601 | cgccacccac ccttcccaga tggtgctgtt ttaaatacgg atctgcaggg ctgaacgcag |
| 3661 | aactgggaga tttattgcaa aatcccggga ggggcggggg ggggtggtgt gcggaacggg |
| 3721 | gaatggagga gcagaattta aaggtgcaac gcttgctttt tccaatcagg cggcaaccgg |
| 3781 | ccggaattat tatttttttc tttctgtctg cttgtctctg gattctaatt caccaagaaa |
| 3841 | gaggtgtaaa tattgtgaca ttttgaggca gcttgatgga tgggaaagaa atcatctgtc |
| 3901 | actctaaatt gcagagttcc ctctccccgc gccatccctt gctagcgaat actcgctgct |
| 3961 | gcctaataca gttgctaggg cttcaaatga atgcatcgtt aagggaatat tatcctttta |
| 4021 | gttgacttgc caatttagtt gacagttgaa tcgagaaaat tgtagatttc gtgtctctgg |
| 4081 | gaggaaaaat gcttaacagt ctaagtcttg taaccttgag gtctttaaca acttaaataa |
| 4141 | acctcaaaag tgtcacgtca tcctctacac acacacacac acacacacac acacacactc |
| 4201 | aacttgtaag atgacatggt ttcacctaaa ctgttgtgga aatgaatagc actttaaaaa |
| 4261 | tggtgcacct gatattcact gtttatgtgt atttacaaag agctcttcag catgaaggca |
| 4321 | agacatttca attgtcctgt ttggaatcag tcagaagact agaaggtgat ggagagaaga |
| 4381 | agggaagaaa gaggaaagag agagaatttt aacctagatg ctattaaatt aacagtaacc |
| 4441 | tagcctactt ttatacccct tggtcttgca tattaatatt tctgtatgtg agattttagc |
| 4501 | ttggtctagc tcccccaatg gagtatacca gtattgattc agatgagaat atgagcatcc |
| 4561 | tgccagtagc tttttcagtg tcattgatag taagacctac aacacagcaa tttttggagg |
| 4621 | atagaagaga atatatataa gggctttgca aactgggaag caggcactcc ataaatggga |
| 4681 | ggtatcatta tgacattctc tttgcacata tcattttcat ttcactgaac cagagtacta |
| 4741 | gttattttaa aacataatct aatgtatatg ctcaaggtag taagtgggga ttttaaaagc |
| 4801 | aagtgattag ttggcttata aaatattatt tttcaattgt ctattaatgt acattggaaa |
| 4861 | gaaggctttt aaagatctaa aatcaacata aataagcttc ccctttcatt tgccagactc |
| 4921 | tttccctatc agatttctga tctaaattct taataagaag agaagctggt gaatttagtt |
| 4981 | tctttccttt tcctggcctg tcctctaggg gaagctttag taagaaacaa cattccaaaa |
| 5041 | tcaggcagtg agcgagagag aaggcaaggg actggatgac cacaaaatag ataatcagcc |
| 5101 | aagaaacaga aatgagggaa aaccagcatt aaagcatgac ttacaaaggg tttttatttt |
| 5161 | gtaattctgt aattttggga ccaggctcaa acttgctcaa gtaacattca ctcgatcata |
| 5221 | ttgcttacaa tctgtcagtt aaaatgatgt ctaactgagc atatttttta ttaaatatac |
| 5281 | ttctctcaaa ggccagtaaa gctactcttt ggttttaatt agacaaacta gtctaaccac |
| 5341 | ttaaataact ctaatgaata tgaactgata tcatcagatt taaaagctct gctgaaaact |
| 5401 | aaatttattc tgaaaagcac tgacttgcca gaaaaatatc tatttttgca gctttctttt |
| 5461 | cactctatgg ataatttaat gagttgctta ttttaatttt acaactgcta cctcagaagt |
| 5521 | atctcaaatt atctttcttt ggctggtgtc tttctctgct gatctgctac tgctgtgtgt |
| 5581 | gtgtgtgtgt gtgtgtgtgt gtctgtgtgt gtgtgtgtat gggcgtgtgt gtctctgtgt |
| 5641 | gtgttttcta gtgggaattt aacaagcagt gagtctctta aatttacatg ccataatcta |
| 5701 | tgtcaagaac attgcgtact acttagcaat aaaaataaac attagcatct agtgaaagct |
| 5761 | taccatcatt gagtgctatg gaaatagagg tcttaaagaa agattaaatt tttcaacaaa |
| 5821 | aaaatttttc ccctttttgg cttaaaggtg attataattt caaaaatatg acatctttcc |
| 5881 | tcttttactt tggaatgtag agctgctgct ttaacaagtg tcttttgaga aagatacacg |
| 5941 | tgtttcataa agattaatac ccttaaaaca ctatggtgca gagagggaag gatgaattct |
| 6001 | ttaaccctgc ctctaatctc atttggcaat ttttggagta ttcattctga ctttttaaaa |
| 6061 | attcaggtgg attttttttc tgctttcttt ccaacattat aaaacaatcc tataagagat |
| 6121 | ttttctgcta tagtgcagac tttatttgta tttcctagta ataacacttt agattcatat |
| 6181 | agtactttga cagctctcta taggtttcat ttgatttcct tatcagtcat gtaaggtagg |
| 6241 | aatcaccaat caccttttac agatgaggaa agtaaggtgc agaattatct aacactacac |
| 6301 | tgccagtaag atgtaaagac taactcagag tctttcttca aattttaagg aaatttgtgt |
| 6361 | ttgttcctct ctagaccatg ctgccttaaa ctccactagg gcatcagagg gagctgtagg |
| 6421 | cattattttc tcctattttg atttatttaa ttaaattaaa aacatttttt acaaatagtt |
| 6481 | ttcaaaattt caggcctaat ggaaagtttt aactagtctt cctaagacag tatttccctc |
| 6541 | tcccacagtt agacactcaa agaaagcagg actcttcctc tagttgacat accatctaag |
| 6601 | tcatagttgc taattcccca aaaaacaaat acaaagaata agaagaacca aagccaagat |
| 6661 | gtacattacc tttacttgtg aatcatagaa tctgggttct gggaaggttc tcagaggtca |
| 6721 | cttagcccag ctcatatctg atacatgaat tcatgctggc ttgttttgtt tcaatcctct |
| 6781 | ggaatttcat cccagtcttg gacactgggt acttgtttat ettggtagat gttctgatac |
| 6841 | ttatgtggat catgagcttg gctgatcatt tcccattttg cccaaaaggt atactttcta |
| 6901 | tagagactct agcattcata attttatttt gtaagtatta attggcaaca cataatttgc |
| 6961 | cattatgtgc caggaactgt attacaatct ggagattcaa gaacaagcaa gaccaaatgg |
| 7021 | tccctatctt ggtggatctt gtggcctaga gacaaagact ggtctcttta tttgctccaa |
| 7081 | tccttagaag gggacagacc tctggactca taagattcct tttttcaaga gaacacagtg |
| 7141 | aataagatta aaagcctgag tttaggcttt acttccaacc gcttgtcagt acccaaaaaa |
| 7201 | gtcaatcagt cactcttgat ctctgcttcc tcacacataa aacaaaagag atgttctcag |
| 7261 | aataaactct aaattccact tccgttccaa gtttgagtga tataaaagat atagtctata |
| 7321 | actatttctt gcagtgtagg ggaaattaag gcctaagtta ccctaacctt tggtgattta |
| 7381 | caattctggg tgggtaccgc aaatttttaa cttgttaaga agtatatcat gaaaaaaatc |
| 7441 | aaagtaacat atttgaaccc agaatagaag gaatctaggt gttgaacctg ttcatttatg |
| 7501 | gaatgatggc taggaaagtt ttaatttaga gaatgatctc aaatttctgc ggattatttt |
| 7561 | taaaagcagg tggtgcgatg gaagacctgc tatcaaattt actgcttatt ttctttgtgc |
| 7621 | aagcagaagt tataattttt caggtcattt ccttttaaaa tcaaaaatat tacatcctga |
| 7681 | aattgcctgg gtctcatgaa taatgcatta tatacacatg ataatagata attagatgga |
| 7741 | caagccaaaa gaaacatgaa aggaagcagg tagcccaagg attgcagaag gtgtgtgggc |
| 7801 | attttgacat ccaggaaatg ctatagatct gtccttaact aactcagcct ggtggagata |
| 7861 | attaagaaaa aaaatgtggg tgtagaaaga ctgcaagcca ttccctggga ttggctagat |
| 7921 | tgctgcagta gttcaaaaac aattggcaca gccacccaca ctagacatga ttgccctttg |
| 7981 | atgaggcagc tattgacttt tataaagatg tcatatttaa aataacttct gatgcactag |
| 8041 | gcataacaga catcattctt gaattctatt ttagacaaat ggcaaaatgt attacaaagt |
| 8101 | attaatttaa aaataaaaaa tctttaaagt ctagtgtcta aaaaccagca gtttagtaac |
| 8161 | atgcaacctc tggatttaag aattcagcct gaagctggga gaaagctgta gcttgtatag |
| 8221 | gacattttga tccactctgg gcatttccca gaccactaca ggaagtaaaa tgtactttgt |
| 8281 | caaagttttt aacctttgag tgaatgttaa atccactcca aaatcttcgc aacctgggaa |
| 8341 | aggtgatcca acaattttcc taaatagcgg cagaaaatgc tctgagatct ttgttcccag |
| 8401 | agtgaatgtt ataatgttat gctatctaga aatttccttg tagcaccatg ctcatcagta |
| 8461 | ccaaaaggag ttagaattga ttcctcccgc ttcaaggaaa tatatcaacc acctcctgtc |
| 8521 | tctaagtaac aaggttactg tggggaaaaa atacacaaat taggtgattg cagaaaggtg |
| 8581 | tcacaaacat ccaaagcctt tgggataggg cattgcagtg tgagtgaata gagaaaagaa |
| 8641 | agagaatgtg ggaaaaaatt gagaaataaa aagggaagtc acagtggagt tctaattata |
| 8701 | caggggctct tgaattgact gttctctacc ttccatgctc attgttgttc tggctacttt |
| 8761 | agtaggaaac aatgatttct tctgctttca ccttcctcct ccgctaagga cttcttactt |
| 8821 | gccaataact tccataatca atgtttaaga attgctctga tgcccagtgt ggtggctaac |
| 8881 | gcctgtaatc ccaacacttt gggaggccga ggtgagtgga tcacttgagg tcaggagttc |
| 8941 | cagaccagcc tggccaacat ggcaaaaccc tgtctctact aaaaatacaa aaaattagtg |
| 9001 | gttcatgcct gtaatctcag ctacttggga ggctgaggta ggagactaac ttgaaccatg |
| 9061 | gagacagagg ctgtagtaag ccgagatcat gccattgcac tccagcctgg caaaaaaaaa |
| 9121 | aaaaaaaaaa agaaagaaag aaagaaagaa ttgctcactg taatgacttt catgccatgg |
| 9181 | actcaactct cttggcagtc tggtaaagct tatgtaaacc cttctcataa aaatgtctaa |
| 9241 | atggggccag gcgcgacggc tcacacctgt aatctcagca ctttgggagg ccaaggcggg |
| 9301 | tggatcactt gaggtcagga gttccagacc agcctggcca acatggcaaa accctgtctt |
| 9361 | taccaaaaaa aaaaaattag ctgggcgtgg tggcatgtac ctttaatccc agctacttgg |
| 9421 | aaggctgagg cacgagaatc acttgaatct gggaggtgga ggttgcagtg agccgagatt |
| 9481 | agccactgca ctccagcctg gatgacacag tgagactttg tctcaaaaaa aaaaaaaaat |
| 9541 | tctaagtgaa tgaaataaat gtataagatt acaaaggaag ccagtggcat tgatgtacag |
| 9601 | ttataaaaac atttaaaata atatattgtg tgatatagta atatatatgc tttttaatac |
| 9661 | attaaataag atctaacagc aaggtaaata ttataatttt gaaataatga taagtatcaa |
| 9721 | tgtattttga aatatctata aaactgacgt gatatgaagg tgtctgtgat gtatactggt |
| 9781 | gagaaagcat gcaagtacta ctgtgtaaca tttcccacac atatttaaca acagaacact |
| 9841 | tgagaagcac ttattaacac agcatagatt cagaaatatt aatttagtaa atgtcaacat |
| 9901 | tagccattgt tgtttccttc ctggcaaaag gaaatcagca ttgggagaaa acttttaaaa |
| 9961 | ttcacatttg ccattagaca agctgtcaag tggggaaagg accaaatact gagaaggcca |
| 10021 | gggtatggta agcatgtttc tattgactga gcttgctatt actctaacgt ttatctttag |
| 10081 | catcaccagc acaaccccat taccctagca atccatcact ccattgaaaa agataaaaag |
| 10141 | ttcagattct ggtcattaac tcagcattgc ttaagatacc tgttctgacc tcactaaccc |
| 10201 | aagagattac tgaaactctt cctgtttgtc attactacac catgggaaat tataatgatg |
| 10261 | tgggatgaca tttactctgc attcatccag tgctgttatt tgttttgtat ttggcatata |
| 10321 | ttacttaact cttaaagtaa ctctcagaga tagattaaga aaactagagc tcagagaatt |
| 10381 | taagtaactt gcccaaacta acacagaaaa tctgaagtgg agaagctaaa cttcaaaccc |
| 10441 | aaagttttct ggttccaaag tccattatga agttgtgcct ccccatctta tagctaccac |
| 10501 | ccagatttaa tctgggtctc ccattatcag atggtttaca tacacatttt cttacaagat |
| 10561 | cttgaccaca actctttgag atggccatga gtctcacaat tcatttccag gagtgctact |
| 10621 | ttagaatcat tttgatcttt gctaaccgat gagagatttt caaatagcta attgtcacct |
| 10681 | accctttttg aagcccagtt ttcataatca taaaatggaa acagtattac aatgttttgt |
| 10741 | taggatcata tacattaata ataaaatcta actttgttga gctcactatg gtgagcattc |
| 10801 | tgcatttcct tagttcattg aatcctcaca acaatctttc taggctaaga cgattatttt |
| 10861 | tcttttaaag ataaggaaac tgaggcatca gtaattaatt aactatctta aattagcaga |
| 10921 | gccaataagt ggcaaagctg ggttcaaacc taggtctgtc taatgtcaaa gccctttttt |
| 10981 | taatcactaa tctgcaaatc actattcaat cttagctttt attattataa ttatcatcac |
| 11041 | acttaaaaca ctatcaagat acagaatgat ccagacataa gtatatagtc actgaagaga |
| 11101 | ttagaatctg aaacttttca cctgcatgtt cttccttcca ctttagttta ttaacccaat |
| 11161 | ggatgatgtc tgactccttt cttaacttgt ttagggcagt tccaagttag ttgacttctg |
| 11221 | agagttattg agtaagaaat gttataaatt gtttggatta ggatttagta tgtttagaag |
| 11281 | ctatttcata agtttgcctt tgcgaactgt tactggctat aatactgcag atgctgtgat |
| 11341 | gaggaacacc ctctccaaag acacacagtg gatgacaaac ctccaaagct aacatgttgt |
| 11401 | ttacagatat ggagaagaag aggatggaca agcacagtct aaaacgtaat tacaaggctt |
| 11461 | atagtccctg ttggggacta gaatgtttat tggctttcct tgtgcaattc aatgctcttc |
| 11521 | ctccaaagga tccactccaa acttggaact ttcctgaaaa tagcatctca tttgggagca |
| 11581 | tgccaggaat tggtgtctgg gtcctttgtg tctttgcacc aactcagaac tctggatact |
| 11641 | agctctagaa actaagctgg gatatattct gggtaaggga gtagcatatc tacttgggca |
| 11701 | tcttcctgat acatttattt catccatctt cctcctagag agcacctcct agaaagatgt |
| 11761 | ggttttaaat gagggattgg atgcatactg gtatgtctta gcacacaagt cagtggtctt |
| 11821 | tgcagagctg ccaaaggcat ataagtaatc aaagatgcgg aagtctatga agagacttca |
| 11881 | tcccacctcc actctgattt attcagggaa ggaccccatg aacacataat ggatttgata |
| 11941 | cgtcccagag ctctgaaagc agcctagcaa aaaaggataa tcttgaagga cattttgatg |
| 12001 | tatgaaaaag tccacctaaa gctttgtcag agataactaa gtaatatgat ggctggtaga |
| 12061 | ctgtaagtcc ttaccttggc tcaggaactg tatatcattt ggtaaactaa acttgtcgtt |
| 12121 | caaatttaga tagaaaaagt accttacaaa tgatctagtt cactgattcc cttcatgcat |
| 12181 | tgaaatcacc taaatcatct cttctttctg agataaggtc tgaatgtgtt gccagcttta |
| 12241 | gcaaactcag tttgtagccc actgacctca tttgattgat tgggcaactg aggtgcacag |
| 12301 | tggtagatct ctcaatttat tcaataaaca attatatggc ccttacgata tctatctgaa |
| 12361 | caatcttggg ctagtgaagt tgcttgccca ggttacatgg ccagaaactg acagttttaa |
| 12421 | attaggacca aagttctttt gactactatc tgggccttaa aataatatca tatgacaaag |
| 12481 | atatttcttc tgtttcctaa tagtcacatc aaaaggaaac aatggacagt ttgtgcaaga |
| 12541 | ttttagttac tttaatgttc aaaataaaat taaaaacaga ttattactaa aacataagca |
| 12601 | taacaacact ttaatagcat tctaatcaga tattattaat ttcaaaatgg taggacaaaa |
| 12661 | ctaattatac tttatacttc ttaaatatcc tatagttact ttatgactat tgagacacta |
| 12721 | gctaaaactt gaaacttcaa gttttcattg attcctatat tattacttat ttcagagtta |
| 12781 | cttcatttgg ttcttttatc tgagattgga caacagcttt atttgatttt cagcgacaaa |
| 12841 | attcttttca ctcctgatcc tccaccccaa gaaaacaaca gctactaata tattttccct |
| 12901 | aaagtgatca agaaataaaa gaggaattct agccaggcgc ggtggctcat gcctgtaatc |
| 12961 | ccagctcttt gggaggctga ggcgggtgga tcacctgagg tcaggagttc gagaccagcc |
| 13021 | tggccaacat ggtgaaactc cgtctctact aaaacttaaa aaatgagcca agtgtggtgg |
| 13081 | cgcatgcctg taatcccagc tacttgggag gctgaggcag gagaattgct tgaacccagg |
| 13141 | aggcagaggt tgcagtgagc caagattgcg ccattgcact ccagtctggg tgacagagtg |
| 13201 | agactctgta tcaaaaaaaa aaaaaaaaaa aaagaggaat tctaaaatta attatatcta |
| 13261 | ttaatatccc tactcttaaa acgttagaaa atgtttgctc atttaaaatt tttattttta |
| 13321 | aaaccacctt atattccaac taaatactct ttggagcaat ttctttgttc ctcatataat |
| 13381 | atccatacat ataattctgc ttttgtgatt aacttttatt actactcttc taaaattgtg |
| 13441 | ctcttataaa catcagttaa ttaagagtaa atctgatgtt ttataaattc tttctagaaa |
| 13501 | cagagagcaa aatcatataa ataacaatat gaatttccaa aagtacaata ataaaaaaaa |
| 13561 | attagaaaaa aattaatcta ggaaatagtc aagaatatgt caaacttgta catacttttg |
| 13621 | agataaattg gcatcatgta gattagcatg attcttcttt atggaattca acttattttt |
| 13681 | actcactttg ctctaattag tttttgtgtg cggacaagat ggaaggtaat ggaaatttgg |
| 13741 | cttgcaaagt agttctaaca tgatctacat ccacaatctg gttataatgc tataagaata |
| 13801 | ttatgtggga atagtagttc aaatcagtat ttagtatgaa cataaaggga caaacaatgc |
| 13861 | aaagctaact taagttgttt acacttggaa cttatttaaa ttaaaaaggc cagtggatgg |
| 13921 | tcatatgttt ggctcattct tctcaaggcc ttcaggaaaa catgcctatg aaataaaaga |
| 13981 | tcctcaatat taaacatttt actgcatttg ggggacacat gaaatctggt aataaaggaa |
| 14041 | gtgttggtct tcatttttct aattcagcat ggaaactatc ttgaggaaaa ctgactatgg |
| 14101 | tcttagtttg tgtctcagaa atatatttag tctgaatcat ggcgtcgaca tctgacttcc |
| 14161 | aaaattggat atctagccgt atagtacctc acctcccaca cacaccaccc cccattccca |
| 14221 | ggtcatgact actgtccaag cagcaaaaaa agaagtaatt tcccagagta catacatggc |
| 14281 | agtgacaacc aaccaaacaa aaaacaatta taggggctgg aatttaaatt aatggctgta |
| 14341 | ctctcaccaa ttcattcccc attccacccc atctctctgt cttcaacttt tatgaaacat |
| 14401 | tatatttgtc ctattcttct gtatcagcat cagcctttcc tatatccaac tagaettata |
| 14461 | acttcttggt gcctctcact ggctgactaa ggtttcagaa gtacctactt acagcaaaca |
| 14521 | cttgcagcag tctctttttg gttacaaagt ccctggacaa tttctcaagg cgatattatg |
| 14581 | aagaggaagt aaacattctc ctctgctacc ccatttcttt ttagagtgct aactttattc |
| 14641 | tatatctggt ttaatgtctt cttaggccaa ttggactgat tttacagaca ccatagaata |
| 14701 | tctcctgagt aatgggaaca atatttctgc tgatcccatg atttggtctc attgggttgt |
| 14761 | taggccataa tggagacata cttgatgaat ttatgaagac ttgattctag gtatcatgta |
| 14821 | ggttagcata attctctttt actgaattca acttagtttt attcacttta ttctaactgg |
| 14881 | attttgtgtg cagaccaaat gaaaagaaat ggttcaattt aggtgaaagg taaagcttca |
| 14941 | aaagtagtgt agtatttcat agaccttacc tttgagagaa attatatcag tatataataa |
| 15001 | gcacctgaga atatgaaagc acaaatccaa tttaaatgtg aaaggtctac aacttgggat |
| 15061 | tttaaatgga gtacagaaaa gccactgttt cttaaacaat tttgttgagg gggaaaacag |
| 15121 | tgaaagctaa atgttctatt caagagttgt ttcttttgaa aataatgctt catttaaaag |
| 15181 | ctaaggacag aagacgtagc tttgttatga aggctcatct ttttattaaa caaccactac |
| 15241 | tttgtctcca agttgcaaag ggaagatttg tcaatctgat tgaatcttcc ctttagtttt |
| 15301 | tcccaacagc tgtgtccaga taattcatga ctcctgtgtt tcctgagccc tggataattt |
| 15361 | cacacacatg tctggtttgg ggctccacat tttcagaaaa atatagaaat ettggaggag |
| 15421 | gtccagggta gaccaaggga aatgattaat gggttgaaag ttggagttta tgaagaaagg |
| 15481 | ttgtgagatc tgatcttttg ctacgagaaa agtctgagtg gtgacttaat aacataagga |
| 15541 | ggttagtaag cagctgttct ccatcttcac taaggttgaa tgaaatgaaa taagatataa |
| 15601 | attgcaacag gaacaaaaat gcattacaag tgaggacttc caagcaccag cattgctgga |
| 15661 | ttctagatag ctccccaaaa gaaggatgtg tagtctactt ccctggtctg caatgacagg |
| 15721 | cctataaata gtgagaaaga tgagataatc tcttaagatc ccttctggac ctatctttta |
| 15781 | taggtctatc tatcatattt agaaaaatta tttgcctcaa acaaaaatta tctgatttcc |
| 15841 | tccctctcac ccfatccact ccttctcttt tgtctacctt ttgtaaaaca ctgctaaccg |
| 15901 | aaataactgg ggactgatta accgtggtgg gccctccccc gcctctaagt gccactccag |
| 15961 | ctttgggagc aagtttcttg tccatcacta ccaccccctg gccactaggg gcatgtttac |
| 16021 | catcatcttt ctacacacca aacctacggc aagggaaaat aaaacaaaac aaaacttcct |
| 16081 | agacttaaca aatttgcaag tgtcaccatg gattaaaata caactcttat gtcctagaat |
| 16141 | atgagcatgt aaagggctaa aatgtatttt atgcatctgc ctgtatcagc ccatagaata |
| 16201 | gcctcctgac agatagtaga tactcagcaa tctttcatca actgaatgac tgtaactatg |
| 16261 | aagtgaaagg caactaaagt tgagaaagtc aggagtttcg gatgtttcca aatgattctg |
| 16321 | tatgccagac taatctaaag cctaacccat tcttcacaac catgcactat taaggatttc |
| 16381 | attctcacca tgcctgtgct atctggaggt agaaagaggg ccagttgcac atcctgctca |
| 16441 | agtccttggt caaaaagacc actaaagagt gctttgtaga ttcatgtatc agaatcacat |
| 16501 | gaaagtaggc caaattctta gtgtgtgttt ttaaaataag actttaggaa gttcacttat |
| 16561 | ttttttctaa attatttttg catattcttc tttttcattt ttttcatgaa gaatttaaaa |
| 16621 | tttggctgta gaaaatctct cactccaaac atcacacagc ctaaataggt gagtctcaaa |
| 16681 | aataagctaa tgttcatctt tcatctgatt caatgtcctg aaaccctttg gtttaaattt |
| 16741 | gttaattctt ctcatggctt ttctcctagc aaaaccaact aataccacag ctatttatta |
| 16801 | ctgtcagctc taacttatgc ccacaatctc acatcccttt tgaccacgct tatagaacta |
| 16861 | ttacaacaag taaaccaaat ttattcttca ttattaattt ttaaatgttc tcagcacaaa |
| 16921 | tctggtaact tggagggcta caagttgata tttctcatat gtttgggggt ttagtctcaa |
| 16981 | cagtttctta atggtttcta tgccgttttt cttgatccaa ctaaatatta ttcccagatg |
| 17041 | ggatcagctt ttgaccctct tgttctactc tcctagtctt ggcccttcta aaagtttctt |
| 17101 | gctgtggttc ctttcttttg tctgccacta atggctatgc ctggttacat aactcctgta |
| 17161 | acaggtgttg accaatttga acacattttg gtatggtatt gagctattct tatggttcat |
| 17221 | aaaaagctta gtgagaacgt aacatctcat gaatagggaa attacttctc ccttaaggtt |
| 17281 | tttctcagga caggcctcat acaagaattt caaggattgc gagtgacata gtttaacatt |
| 17341 | ggaccaggcc tttcaaatta tccaggatga gtttgaaaac acctgtgcca ctctgctcaa |
| 17401 | cagcagagtt ttctgtttac taagtatttt ccctatgcta attacggaaa gtttcaacag |
| 17461 | tttttttagg ccaacttatt tgatgctaga ctagacaact tatttttttt ttcttgcaag |
| 17521 | gaatactgaa ggtaggagta actaggaagc ttaaataaac ataaatataa aatgcttata |
| 17581 | gtgatagaat tgacctcagc caattaaaat tattaataga aaaaacatgt caatgtcaag |
| 17641 | cctactacct ctgttctcac ttgagtaatg aggattagtt tatatttccc gacaagaata |
| 17701 | gatgggaatt caaatttctt cctgaccttt gttccccctg gaacattggg ttaggatcat |
| 17761 | attagaacat aaccaaaaag aaataaagat tcagacgaat tcacaattaa tttttaagcc |
| 17821 | ccacaaaagt gaaataggta gcattatttt ttcaagctgt gaaactttcc ctcattttag |
| 17881 | taatagagaa aatgttcaga ttataaactt ggaaactttg ctcctaacat atcaattatg |
| 17941 | ccagaggcca atttttaaga agaagagaaa tgcatgctct atattctcag catcatcctt |
| 18001 | gcccacaata gggaaataat tttgtaaaat gtttgatttt agacctccaa aattatctct |
| 18061 | atatgctacc tgaattaagc aaataaaaaa taatatttag aattccatgc aaggcactgg |
| 18121 | tacaattttg tttatcttgg cttcattgtt tttgaatgta agatgtactt ttaaggcaaa |
| 18181 | taagtacatg ttttaagctg gtcgcataca gtattggcaa tgctataatc acaaatcaga |
| 18241 | aagtttggaa atgcttacaa gtgttaagag gtgtgattca tcatggttat ctgaattggc |
| 18301 | atctgatctt cttttctttc taaatatccc tgacatttct gactcctctg tcttttcctc |
| 18361 | agtaaaactg caccacacac tggaaagcga agatacacac atttatttat ataatgtcaa |
| 18421 | gggagagtag gaataagaag attggccata gacccaccca atcagagtct gggaaatgag |
| 18481 | aacacttttt ccttcagcag aaatgctgac gtgccaatgt gaatttagca gaaaaaagat |
| 18541 | ttgccataac ttctaagtga gcagccttca gaatgctagc ttagattcct ggcattaact |
| 18601 | tgccaggtat tttttcagga aggaaataaa ttacaattga gcttaaaaac ctgagggtag |
| 18661 | aactcatttt caagcaaatg tgaagcatca gtttgaagtt aacaaagtta aagtttggag |
| 18721 | tagggttcct ccagtccttt ataatgtagt acaagtattt tttttaaatg tataacacta |
| 18781 | gccttttaaa ttgtattgtg ctactaaaag aaattgtgcc tgcattcatc ttacaacctg |
| 18841 | ggaaccaacg cagagggtct gtggggtagc ggtatccagc ttcatgccct ctgtccttta |
| 18901 | ttgctttctg gttagcctgc gtatttcaca tacattaaat attccacaat aaactctgcc |
| 18961 | atctgtgctg tagggtagtt tgtattggtc atgtgctctg tcaagttgac agaggtgcaa |
| 19021 | agctaaatgt gtgacactcg aagaatatgc atatatttga ataatttgac tatttagtcc |
| 19081 | aacaatttgc aaaggcgctc tgaatgatca cacattctga taacacttcc aaggaacaga |
| 19141 | tagcttcact tagggggtgg gggagatgga agcagggtta tttctagcag gaattcttga |
| 19201 | gttcactgaa gtcttgtccc tggtacttca ctgtgtgaac gtgggtaaat tatttcctgg |
| 19261 | cagaggatcg gattcttctt ttataaaacg ggtaaataat ttctgtcact agtctttaga |
| 19321 | agttctaaaa tagctaatgt tagtgaattc attttgctaa ctgtaaaccc ttaggtaaat |
| 19381 | tgaactgagt atgtaataat attatatatt cagttcaaca gcacattctt ggtaaccaca |
| 19441 | agagggtcca ggaaaggaaa ctgtttataa atctttccct ttagcaaaat taatgttgga |
| 19501 | gtctttaggg aaattcttac agcaatagtc ttcgcaatta ttaggtcaaa cccctttgag |
| 19561 | attacagaaa aacgcacaca cacagaaagc tgcctgcaga atttgggtgt gggcttggtg |
| 19621 | ggagattcct ctgataccca gtgttgtacc cccaagagag tgtttctcaa agtgtgactt |
| 19681 | cagattgtct gcattcgaat tgcttgtggt atttattaaa attataactc ctgggccctg |
| 19741 | ccccacccct actaaatcac aatttcagga ggagggacct tcattttaac actcacccag |
| 19801 | gtgattttta tgctccgagg aggtccaggg actccaagtt aagtacggta ctgctgtctt |
| 19861 | attctttatt ctaaatttta aggtctgcac aaattggttg aactaatgag aagaaaattc |
| 19921 | agctttaaag cagaaacaca ggtagacggt tgacagagtt catcaaatgg ataattgaaa |
| 19981 | atgtcctctg gaccctagcc atataagttc tcttcaaggg tcttggctac aggcaaatga |
| 20041 | gaacccgaaa ggctatttgc tcttttgctg cgggcagtgg tgggggtgga gggcggggga |
| 20101 | ggattaactg agccagttct gcccccaccc tcgaatcacc tacccccact ctggttaaag |
| 20161 | cagaagactt tttatttatc ttggctgccc tggttcgtta ttaaaagggt tagcttatac |
| 20221 | gtgtgtttgc tggggctgga agtgaaaaca tctgcaaaag catgcaatgc cctggaacgg |
| 20281 | aactcttcta ataaaagatg tatcatttta aatgcgctga attttgattc tggtaattcg |
| 20341 | tgcactagag tgtctatttc gaggcagcgg aggtatcata tgacagcgca cgtcaaggca |
| 20401 | ccgtggagcc ctctcgtgga ctcccaccca ctttcccatt caccgcggag agggctgctc |
| 20461 | tcgctgccgc tccccccggc gaactagcat gaaatctccc tgcctctgcc gagatcaaat |
| 20521 | ggagcttctc gctgatgggg tgcgagtatt acctccgcca tgcaatttcc actatcaata |
| 20581 | atttaacttc tttgctgcag aacagaagga gtacataccg ggcaccaaag actcgcgccc |
| 20641 | cctcccccct ttaattaagc gaagggaacg tgaaaaaata atagagtgtg ggagttttgg |
| 20701 | ggccgaagtc tttcccggag cagctgcctt gatggttact ttgacaagta gtgactgaaa |
| 20761 | aggtgggttt gttttctttc tttctctttc cgtttttctg tttggtcggc tagaaagcgt |
| 20821 | gtggctttag cgaggtctgt cattgcctgg gcttcctggc tggaacaagt aacttggtgt |
| 20881 | aacgttatct gggggcgttc atcaataaaa aatgctgtta ttatcttgat tgaattccta |
| 20941 | ttaggcaaac tctagagagg tcagtgcgcg aactctgttt aagccggcgt gtttaaggca |
| 21001 | gcagagtaaa ccaatagccc ccatgctctg tgcgatttca ttgtgtgctc gcgttcgcaa |
| 21061 | gctccgtagt gcaggaaggt gcgggaaggt gtgtctgtgg cccgggaaac gcacgccctc |
| 21121 | tcccagagaa cttgggtgct gggatgggga ggaaggggag agttgaaagc taggggagcg |
| 21181 | agacctcggg gcgtgcgatt ctcactcgct ccctcccgcc ccagcgccca cagccggggt |
| 21241 | ttctgcagag ggcgcgggac gcggggttcc ccggggctga ggctggggct ggaacacccc |
| 21301 | tcgaagccgc gggcgtcctg tccaaggcgc cccaggaggg cgcaggactc gcagggcgat |
| 21361 | gtcgcggggc cctaggggag gaggtgagga caggccccgg gggagcgggg agttccgggc |
| 21421 | gcccctcggt tccccgcgcg aggaaaagac gcggcgttcc ctttaagcgg ccgcctcgaa |
| 21481 | cgggtatcgg tagcgcgggc gagcggggag cggggggcgg ggggcggggg ggggggggcg |
| 21541 | gcgccgtttg accaatcgaa gctcaaccga agagctaaat aatgtctgac ccgggcgcaa |
| 21601 | ggcgcagcct ggagctccgg gtccccgacg ctgccgccgc cgcgcccggg cgcacccgcc |
| 21661 | cgctcgctgt cccgcgcacc ccgtagcgcc tcgggctccc gggccggaca gaggagccag |
| 21721 | cccggtgcgc ccctccacct cctgctcggg gggctttaat gagacaccca ccgctgctgt |
| 21781 | ggggccggcg gggagcagca ccgcgacggg gaccggggct gggcgctgga gccagaatcg |
| 21841 | gaaccacgat gtgactccgc cgccggggac ccgtgaggtt tgtgtggacc ccgaggtagg |
| 21901 | caagcgctgg gaatggggct tggtgcagga gctgcccgtc cgcgggagag agttgactgg |
| 21961 | gggatccccc accccaaagt tgtgggacga ggccagtctc cttctttcct cccctccggt |
| 22021 | agaagggacg atttggagtt actcttgggg agttttctcc cccatcccac aacccagaag |
| 22081 | gtcagccggc accaccaggg aaaaagggac ccggggaagt cacgaagtag aggagggaag |
| 22141 | gcctggagga gacccagagc tgcgtgatgg gagcaaagac ggcgacccgg ggatccctcg |
| 22201 | cagccctccc ccagcccagg agtagtcgag agagacttag ggggccagag ctgtcgaggg |
| 22261 | tcctgactga ggggagggtg ctggggctag gctaggaatc cttccagggg gtgggtggtc |
| 22321 | cccgcgccga cttgcggggg gagtgggagg gaagcttgcg ccttcagccc gcatcccttc |
| 22381 | cccggagctg cacacggcta cctgctcccc aggaattgag actgaagtgg acttacaagt |
| 22441 | ccgaagccaa tgtagcttgg aaaacttggg aggcggaatt cctaccgctg ggaactgaaa |
| 22501 | gggtctgcga cactctcggg caggccgaac ccacatctct acccatcctg cgcccctctt |
| 22561 | ctgaagcgcc ctccagggaa gttaagagtt ttgactttcg gggagtggtt gggatgtacg |
| 22621 | tgggggattc ttgactcggg ttagtctctg gggatgcaga gccgggaaga ggaatgggtg |
| 22681 | agtgagttac tcctggaaag aaatagctga ggattggggg ctctgtgcct gacgggcaag |
| 22741 | aagaagggga gattacagac taggggcatc cctaaggaag aagcctcggg gctgcgaggg |
| 22801 | tgaactggag gatgcagtgt ttgtgtgttg ggggtagagc ggggatgagg gaccggggtg |
| 22861 | gaggggaggc gaggaggagg aggggaccca gagaacgaag ctagggaagg tagagggtgc |
| 22921 | cctctgccgg ccatgctgcc aagagcagct actgggggcg ggaggctggg ggtggggaag |
| 22981 | tggtaaagga aggttttgcg ggatccctta gagagctggt aggagggact tgttgaatgg |
| 23041 | tgctgctgac tccagctcgg tggggcgtgc gactcgtcgt cggtggattt tgactcctcg |
| 23101 | ttcttgtttg gcttctatgc aagttttcct cgcgctgggg gagctttgat aagcctcgat |
| 23161 | tggcggtgtg ttagggcttc ttggatctta ttttagggtc ctctagttat cctgcactta |
| 23221 | ctccttaatg tcagtagcaa ccaaagaaca ttttccgaca agcacgcagg aatgttcttg |
| 23281 | gccagaagca aagaaaggca tatttctgag tgtttattaa tcctcctagt aatcttttaa |
| 23341 | agcaaagtaa tatgtaattg ggaacgttga ttttctaact gcatataaaa ggcgacatga |
| 23401 | tattaaatga gacccctccc tactgactca atatcctgca aaatctctct ctccccttta |
| 23461 | ttattatgga aaaatctatt tttatatgag tttgttgtaa ggtcaaaagc cattttggtc |
| 23521 | ttacaatttg atatgtcttt acattttaac ttattgaggc ataattacag atttaatttg |
| 23581 | tatgaacgtg tgtgccttca atgcttatct catgcaacat aatttttagg ttggagattt |
| 23641 | ctgatgttat ggcatgtagc gtttcaaggc attacacata ataggtaaca tagcatgttg |
| 23701 | aaattacacc acaaagtttt gaccctggga acagcacctt ttaaaaacaa tcactaaact |
| 23761 | cctgttcctg ttttctgatt ttgcaaatgc cttgcttaag actttttttt tttttttttt |
| 23821 | tttttttttt ttgggaaatt tacctctggg ttagcaggag aggtaaaaaa aaggaaagag |
| 23881 | acacttgttg aaatgtaacc ataaccttta ctggaattta aaacatgttg gtcaccatta |
| 23941 | ctggaattcc agggccataa agtcgttgtc ttttttttct tctacttcat tttgtaaaat |
| 24001 | gtgataaatg ttggtaaata tagaccagta gtaagtatta tgacactaaa agcattatgt |
| 24061 | atgtggaact attttaagtt attacagaac attttctatt tataaatgat ataagcagaa |
| 24121 | agaaatgatt tccagataaa caaggcttac gtacatgttt tgaagcatta gaacattgca |
| 24181 | gacactctta gacatcacat tttttaaagc aaaataacag taatttttca catacctttg |
| 24241 | gagcctttca tagcccattc agagctgagt tagtagctgg aagtttcctt tattttaagg |
| 24301 | tgatatttta aaaccattta acatgtatag taggtcaaca ttggtgcatc cagaaaatga |
| 24361 | agcatttagg aaatctgttt cagtgtcttt tcaatgtgtg taacttttac ttgcaaacca |
| 24421 | atggaaccaa gaaagtcatc atttgcctaa aatgcagtca tcacctcaaa tgattcattt |
| 24481 | atactatgtg agttaattgc cttcatctca ttaatggcca aggagggaag ggaggtcctg |
| 24541 | gggtatttct tgttcatttt gactcaccag gagggaaaat cctgtaaaaa aagaaatgca |
| 24601 | aatttctaaa atcctggctc aaagtccgtg ggtttcctgt ttaaaagggg cgccatgaaa |
| 24661 | atgtaagcta ttcccttttt cctggaatct ttaagagtcc cagcttttca atagtcaaaa |
| 24721 | tgtagatgat tgatatcatt tcttatatga atagcactgg tttgtagttc agcacgcaca |
| 24781 | gtgagctggg cacgcccacc tgatagtata gcagagaact tgtttacatt ctttttacat |
| 24841 | tcatcttcta aaacctgggg tgctctctct ctctctctct ctctctctct ctctgtgtgt |
| 24901 | gtgtgtgtgt gtgtgcgtgc acgtgcgcgt gtgtgtagag ggggagagag agagagagag |
| 24961 | aactgtgaac tgtgaaatat aacacagcca gcagctttgg gtctcaatcg tagacttact |
| 25021 | cttaaggaaa tttacagaat ggaaaggtca tgttcaagta gtttattaac attttgagat |
| 25081 | gtaggaaatt aatcccggag tacagaagaa caatttcaga cttcctgaat aaaaacagac |
| 25141 | agcatagaga gtggatgata gctaaactct gaatatcttt tgagaagaaa ggcactccca |
| 25201 | tttcaggtgc ccataatatg gatttgattt tagtgattaa aacattaatt ttcaacttgc |
| 25261 | atctccctgt gtggaagagt tcaatttgtg tgaggggtct cgcctatcca acaaaagtga |
| 25321 | atatgtccct tttatagggt aattgctaac ttgtctcaac ttgttttcaa acaattgtta |
| 25381 | tagagcactc agtttccact aattgcaaaa ttgttgctta attgaaggac tctcagccat |
| 25441 | ctagtgcagc cattcagcca ctggcaggct ctgtgatctc aaactgtgaa ttgcatttta |
| 25501 | aagaggaatc gaggagagaa ttctgtggaa ttctaggttt taagtgctgg ctgttgttca |
| 25561 | atggaagagg aaatcatttg aacaagaatc gcatcaagtt gtgttgtgat aaattttctt |
| 25621 | tattaggatg aataacatgc acagatgagc ttcaaaagtg aatgagcaaa cttactggtt |
| 25681 | acactctgca tccatttact ctgtttagta tggagtaatg ttaggcaata aatgatgctg |
| 25741 | gcaaatgaaa tccgtatgtt atttgcatgt ggtatttaaa cctaggaaac atagagtggc |
| 25801 | tttggtattt gtaggcttag tcatgtgtgt cctaaacgtc ctcttaaact tctacttaag |
| 25861 | gcatagaatt atttaatcct aaataatttt atacttaagt gcctcactgg atttccagaa |
| 25921 | tatttacact gtaaagattt agaaaggtca tgaacccaat tattgactat atggaatcat |
| 25981 | tattgatggc agatgcaaaa tggagctcac taatgtactg acattgaaaa ccttttgcag |
| 26041 | gggagaggag ggggagtggt aaatgtgtgt gttctttaag tggaacagga aggtattctc |
| 26101 | ttttctgtag aaaaatttga gtatctggtc agataagtgt ggaagctttc atttaaatta |
| 26161 | agtatttaag ttcaagtaga agctctaggg cacttatcct cttgatgaga caaatcttat |
| 26221 | caaatatact agatgctaag aagtggctca ttgccctgat gtctcattta tagattgatg |
| 26281 | tttgaggatg ggttgcatta agtgagttag ggggctgagt gtgggacagg agaacgattg |
| 26341 | gaaggaagca aagtaaattt acaagcttta gtgacagcca taataaagta aaagtttatt |
| 26401 | tccagagagc ctagagagta aggaacgtta tatagttttc cccaaaggtt cacttgaaag |
| 26461 | aacttttcat tggttgtcat ggtagtaatg tcctgatttt gaaatctccc agaacctagt |
| 26521 | agctcttaaa catgctttca tcttggttcc tttggtctga cggaaacttt atgacgaccc |
| 26581 | tctgtgtttt tgacatgcct ctgcattttt ggagagagga ggtcaggcaa gggaggattt |
| 26641 | cttaaaacta agacagtata gtaaggaaac ataaaattat atgataaaaa atcactgaac |
| 26701 | ttcaaattga cttactgaaa taaaacctag aaggcaacct gtcgtttaat tacaactagc |
| 26761 | ttgtataaaa ttaaaattta taaaatggga attcaaagaa aataaacggg cagttccaag |
| 26821 | taatttaagc aactcaccaa aaattgaagt aatagtgcca cctagagaac aaaatcacca |
| 26881 | gctttactag ccaaatggct tatttccata tgaaccattt ttccaacgct acagttacta |
| 26941 | ggatttcctt gttaccatat tcagatcttg tgagtgtgta tgggggtggg ggttgcatgt |
| 27001 | ggaattacag atgaaatttt aaaacaagca gatccacaat ttgatatatg cactaaatcc |
| 27061 | ttttaacgtt gtaatgtagc caaatgtaga atagcatgcc aggaatcaac ggctagcatc |
| 27121 | ctttttaaca tttattattt tcatggatat gtaccaaacc gaaccattga gtataaaggt |
| 27181 | tctgatttta tttatttgct acaggcaatt cattatactt tctgagatac aataacacca |
| 27241 | aataatttga gtagagagac ctttaagaat gttttcgatt tatgatctac ctttaacttt |
| 27301 | aatgtactca gaagatgtga gaataaaata aagtcaaata taagcaagat tttaaacaca |
| 27361 | cacacaaaaa acaaacaaac aagaaaaagg aagaaaatta taaggattgc cttaacctta |
| 27421 | gaatagatga aggtatacat ctgagccagc accaaaaaaa aaaaaaaaaa aagttatgga |
| 27481 | accaggaacc aataattaca aattgactta aaattcttgg atgacaaaaa tctatattta |
| 27541 | gttcattttt gcatgcgccc acaacagcat ccaaaacagt tctggggagg cactttgata |
| 27601 | aatgttgctg aatgcactaa tagattgatt aatggctgct tcagattatc actagtgatg |
| 27661 | tagacagaaa cttcatgaaa atggtttgtc ttgctggaag aaaggcagaa attggaggaa |
| 27721 | aaggtttaat aatatttttc cccagtacct attataaaag tcatttagtt ggcttagttc |
| 27781 | tataatttct tatgtgtaat ttgattcact tatgaaattg tgaatatatg aaatgttaaa |
| 27841 | gttgatttag acagcaacta taagcttgtg gattttcttt taaatgtctt caaattttta |
| 27901 | aatgccagtg gagatgccag cgactgtgct tcagggagta gaatatagta tatcttaaat |
| 27961 | ttgtgccaat ttctggtaag cagagaaaaa attgcatgat aaccaaagaa agtcatattg |
| 28021 | tttgtgcttt gtgttattca tggaagcaat caggtgcaga aaactttctt tttcagaaaa |
| 28081 | aaaaaattac taaaataaag gtgcgtgtgt gtgtatgcac atatatctaa agggagagag |
| 28141 | ggagaaggaa acttactaaa taaaattttt gccacatggg atttagtcta atcagtcttg |
| 28201 | gttttggagt tgctatcatc agtagttcca ttttgtgatt ctttctttct gccttcatgt |
| 28261 | gcctttgaaa actgaaacta tgcccaaatt aaaacaagtt tttctgtctt ttcacatgtt |
| 28321 | cacttatttc ttgaatgtgt ttttaaacac agacaaactt cttttacatc atgtagaatc |
| 28381 | tgaaggtcga gaaatttgca gtcattttgc tggagagaga tgcttggcgg agtcccaggc |
| 28441 | cacattccta ggccaaactc tcgaaggtat tcctcttatg caacattggg aaaatacatc |
| 28501 | cagcaccgac atgttggctg ataatgtgtc tgaaggcaca gacgatatgc ttatcatatg |
| 28561 | aaacataaag ccagcagata ttgcagacat tctgttgaat gatagaatct ggatcattta |
| 28621 | catttactta aatgtaaaat actatgatta agtacaaaaa aatcaattta ggagagaata |
| 28681 | gagagttgcg ggcacggttt taggggatga cttatcagca gattgtagaa aggaagcttg |
| 28741 | aatgttttaa attaactgca agttcagtat aagccagtgg tgtgacaaga ggctgttatc |
| 28801 | atagctactg aaattttggg ctgcactgct agaaatataa tactgaaatg gagaagctaa |
| 28861 | taattcttca ctttttaaat agactgtatc tagaatatta tcatcagttc aaggaaatga |
| 28921 | aataagttgt tttaggtaca tcatcgataa attagtgtac attcaaatca ctgtgaccag |
| 28981 | gatgcatagg gaatttgaaa gcattgcatg tgagcaatgg ttgaggggac ttggaatgca |
| 29041 | tgacttaggg acaagaaaac ttaggctgga atggcaagtg gtttttgaat gttgggttga |
| 29101 | gaagaattct aaaactgtga aggattagta aaaataacat tcagattgct aatgcctact |
| 29161 | gtggctggga gattagagtg tcaacatgtg tgatgtattt ttgacatcct tattttgagg |
| 29221 | atgggcttca aagatttgac gaactgtcat aagtgtaatt tgtgttgctt cagacagcag |
| 29281 | ttctagaacc aatgatgtaa atttagatac tctacatggt agttagaaaa ctttccatta |
| 29341 | atttaattta gcaaatattg aatgctcact gcatacagag cactttatta gaggaatata |
| 29401 | taataaagaa aaaagaggtc tggtgtggtg gctcatgcct gtaatcccag cactctggga |
| 29461 | ggctgaggtg ggaggatcac ttgagcccag gagatcatta cgagtctgga caacatagca |
| 29521 | agaccccatc tttacaaaag acaaaaaaaa ttatccaagc ctggtgacag gcacttgtag |
| 29581 | tcccagctac ttgggaggct gaggcagtag gatcgtttga gcccaggagg ttggggctgc |
| 29641 | agtgagccgt gattgtccca ctgctcttca gcctgggtga cagagtgaga ccccgttgga |
| 29701 | gggaaggaag ggaaggaagg aagaaaggta ggaaggctga aatgaaaccc ttttagaaat |
| 29761 | gacactaaaa tgggaggttg gagtaaggta ttttctgaag tgcctttgta cttgtttttt |
| 29821 | tctaatgcat tggccataag tctgctcctt atttatagtc cataaacaat cctaatgaga |
| 29881 | acagttatat atttctgcct ttgaatcatc tacttgaagt gtttagcatc atgaattgag |
| 29941 | tatcagaaat ccctcccatt tctttgcaaa gcgctgtatt ttacttttcc ttatttgtat |
| 30001 | acagattctc aaaattggct atttttcctt tgggttagac agaacagaat gtctggaaaa |
| 30061 | aaaagttctt atcaaattca ggtgcccaaa ttgcttaaga aattaacttt tgaggttata |
| 30121 | tttttttagg gttcagtagc taaactaaga aaacttctca ccgttcacct tcacttttgg |
| 30181 | aaaccacaaa atcttcagat attacagttt tccaaagagt ttctcttttt aaatataaac |
| 30241 | taaaaggaat tgactcctcc cccaactccc tcaggcctca gcatggatag agttactttt |
| 30301 | tttctttaat aatttattta taacttattt tgctcttctg tagaacagct ggagattaag |
| 30361 | caacatggcc atgacataaa atgcaagtta gaccataaga tgagcagccc actccaagta |
| 30421 | tgaatgagta cttattcttt gtgatctctc atactgcttt taggtattaa tagtgtcagt |
| 30481 | cagcaaagca aacagtttaa tatttacatc tcctttagga tatcatatag tttatagttt |
| 30541 | gtatgtgttc ttgcgtgtat gttttcttct gtttcaaatt cttttttctt aaagtaagaa |
| 30601 | tgttatatgt agcaaatggt tctttcatta attcatttgt taattcaccg tgcattaatt |
| 30661 | gagtgcccag tgagtgtcag gcactgggct acttggattt cttttccctg tattgatcca |
| 30721 | tatatcttca ggtgctcctt gatatggctc tccattgact ctctcatata aggctttcat |
| 30781 | tacctattca catactccct cccaaagagg actggtccaa aagtaaaatc ttgagcaagt |
| 30841 | tctctgagtt atttcagaac ttcttgcccc caaactgtat ttttaattat tgactggtag |
| 30901 | cattttggaa taacttacct ctctttttta aagattgaag ttctttatcc tctctgattt |
| 30961 | tcaggtgtca gtttgtgtac aaattgagac aataaaaatg tttgttagac attttcttaa |
| 31021 | agcattttgc tatgtgagaa tctttcatga agaactcttt ttaacaatga ctctatagca |
| 31081 | gaagccacag tagagggaga actactgaat caaagatggt gtttgagtct atgattttat |
| 31141 | gatggatttt tttttttttt acatacaagg atatgcatgg gtcttttagt attcaggaat |
| 31201 | ctgttcttca cttgacagta tttataaatt gtgtgtttcc ccctaaaaaa acttaaattg |
| 31261 | tgagaatgct tccatttact agaagttggt taatgattat gccaaataag gaaaataaga |
| 31321 | cagaaaaatc agtttagtga atactatttt gcctttaaat ttagtaattt agtaacagta |
| 31381 | tctctttggg gtttactaga aaccactttt taatccaata ggtctctttc attgtgaagt |
| 31441 | caggaggtga ttttgcttaa atgtgtagta taggaatcta tatgtggtgt tcaaggatca |
| 31501 | tgtaaatatg ctgatataat cggagcacag tttggcatca ttaactcaga aatatttaaa |
| 31561 | ctcttgctat acaacatgga agcaaacttg tgcatagttt gtgtgtgtgt gtgtgtgtga |
| 31621 | atctcaaaaa aagaaaaaaa tcacaggatc aggaagtcgg aataggtccc acttttcttc |
| 31681 | tagtaccaaa cctacagcca tgttcctagc cttctctttt actcccaagc aagacagaca |
| 31741 | ggcaaatgac catcctgctg cccatttctg tgtatattca cttgcattga gagttgtatt |
| 31801 | cacctgcttg ttgagagtat tcacaaatgg tacctgataa agtagatact tctttaaaca |
| 31861 | tgtgaatttt tttgcattgt ataatgttta gaaataatca tgtataaatg gttgaatatt |
| 31921 | aatacaggat tgccttatca agtattttat taatcattaa aatgtggtgt cattaataca |
| 31981 | atttatttta agtgcttttc ctaaaatacc agattatttt tctgattttc acatccctga |
| 32041 | caatgacttt cttaaacttg gtagccagga acagaaaacc taacactgca tgttctcact |
| 32101 | cataagtggg agctgaacag agagaacaca tggactcagg gaggggaacc acacacactg |
| 32161 | gggcctggag cagggggcag gggaagggag agagtgcgtc aggacaaaca gacaaatagc |
| 32221 | taatgcatgc gagccttaat acctaggtga tgggttgata ggtgcagcaa accaccatga |
| 32281 | cacatattta cctatgtaac aaacctgcac attctgcaca tgtatcccgg aacttaaagt |
| 32341 | aaaataaaaa ataataataa aataaaataa acttagtagc atctattgtt ccagagcctg |
| 32401 | taattgctct tcaggcagtc tcacataaaa acctaggaga accttcactg tcactgttcc |
| 32461 | atgaggtgtt aggaaaactt gctctactgc agtgccccag taggcattgg tactgagacc |
| 32521 | aaaattcagc tggtttgttg ttactacgat tcctacgtga tttcacttgt catgtagaca |
| 32581 | agattgcaca cttcaataat aatcttgtcc aaatgtgtgg tattccatac atttttaaaa |
| 32641 | tgcattcaca tatctcattc catttgatcc tacaaataac tctataaaaa agattggcag |
| 32701 | acattatttc tatataacag aggaggaaac tggagcttag agaagctaaa tagcaatcca |
| 32761 | aaatgcacag ctgtagaacc agagcaagga tgatagccca gtgacttcac ctaacctagt |
| 32821 | ccccttacca ccactccagc tgtctataac caaaacctgc agtattcaag taagaaacca |
| 32881 | tatcttgccc ttgatgcatt aatgtgagac ctggagcagg aacaggctga tattgtcacc |
| 32941 | ctggcctact gtccaccttt gtctccagca gagactggta cccttctgtg tgccaaggaa |
| 33001 | taaagtggta atgggaagat taaaaatgtt ttttccaagg agttttttaa tttaattttt |
| 33061 | ttaaaaaaga aaaaactctt agagggaaaa atgaatatat gacttttgat gtattgttcc |
| 33121 | ttagtaactt agttataatt ttacttaaac ctgagactct tgctaagtga atgattagaa |
| 33181 | atattaggtg gctggccaga tggcaaatag gaacagctgc agtctgcagc tcccagagag |
| 33241 | atcaatgcag aaggtgggtg atttctgcat ttccaactga ggtaccgggc tcatctcatt |
| 33301 | gggactggtt agacagtggg tgcagcccat ggagggtgag ccaaagcagg gtggagcatt |
| 33361 | gcctcactca ggaagcgcaa ggggtcaggg gaactccctc ccctagccaa gggaagccct |
| 33421 | gagggactgt gccatgaggg acagtgctat ctggcccaga tactacacat ttcctacagt |
| 33481 | ctttgcagcc ggcagaccag gagattccct tgggtgccta caccaccagg gccctgggtt |
| 33541 | tcaagtacaa aactgggtgg ccatttgggc agacacccag ttagctgcag gagttttttc |
| 33601 | tcatacccca gtggcacctg aaattccagt gagacagaac cattcactcc cccggaaagg |
| 33661 | ggctgaaggc caggcagcca agtgatctag ctcagcagat cccaccccca tggagcacgg |
| 33721 | caaggtaaga tctgctggtt tgaaattctc actgccagca cagctgcctg aagtcaacct |
| 33781 | gggatgctcc agcttggtcg ggggaggggc atccgccatt actgaggctt gagtaggctg |
| 33841 | ttttcctctc acaatgtaaa caaagccact gggaagtttg aactgggtgg agcctaccac |
| 33901 | agctcagcaa agcccctgta gccagattgc ctctctagat tctccctctc tgggcagggc |
| 33961 | atctgggaaa gaaaggcagc agccccagtc aggggcttat agataaaact cccatctcat |
| 34021 | gggacagagc acctgggaga gggggtggct gtgggcccag cttcagcaga cttaaatgtt |
| 34081 | ctttgcctgt tggctgtgaa gagagcagtg gatctcccag cacagcactt gagctctgct |
| 34141 | aagggacaga ctgccttctt aagcaggtcc ctgaccctcg tgattcctga gtgggagaca |
| 34201 | cctcccagca ggggtcgaca gacacttcat acaggagagc tctggctggc atctggtggg |
| 34261 | tgcccctctg ggacaaacct tccagaggaa ggaacaggca gcagtctttg ctgttctgca |
| 34321 | gcttctgctg gtgataccca ggcaaacagg gtctggagtg gacctccacc aaattccagc |
| 34381 | agacctgcag cagaggggcc tgactgttag aaggaaaact aacaaacagg aatagcatca |
| 34441 | acatcaacaa aaaggatgtc cacacgaaaa ccccgtacaa aggtcgccaa catcaaagat |
| 34501 | caaacataga taaatccaca aggatgagga aaatccagca caaaaaggct gaaaattcca |
| 34561 | aaaaccagaa tgcctcttct cctccaaggg agcacaactc tttgccagca agggaacaaa |
| 34621 | actggatgga gaatgagttt gatgaattga cagaagtatg cttcagaaag tgggtaaaaa |
| 34681 | cagactcctc caagctaaag gagcatgctc taacccaatg caaagaagct aagaaccttg |
| 34741 | aaaaaaggtt ggaggaattg ctaactagaa taactagttt agagaagaac ataaatgacc |
| 34801 | taatggagct gaaaaacaca gcacgagaac tctgtgaagc atacacaagc ttcaataact |
| 34861 | gaatcgataa agcagaggaa aggatatcag agattgaaga tcaatttaat gaaataaagc |
| 34921 | atgaagacaa gattagagaa aaaaagaatg aaaaggaagg aacaaagcct ccaagaaatg |
| 34981 | tgggactatg tgaaaagacc aaacctacat ttcattggtg tacctgaaag tggcggggac |
| 35041 | aatggaacca agttggaaaa cactccttag gatattatcc aggagaactt ccccaaacta |
| 35101 | gcaagacaag ccaacattca aattcaggaa atacagagaa caccacaaag atacccctca |
| 35161 | agaagagcaa acccaagaca tgtaattgtc agattcacca aggttgaaat gcaggaaaaa |
| 35221 | aagttaaggg cagcgagaga gaaaggtcgg gttacccaaa aagggaagcc catcagacta |
| 35281 | acagtggatc tctcagcaga aaccctacaa gcctacaagc cagaagagag tgggggccaa |
| 35341 | tattcaacat tcttaaagaa aagaattttc aacccagaat ttcatatcca gccaactaag |
| 35401 | cttcagaagt gaagtagaaa taaaatcctt tacagacgag caaatgctga gagattttgt |
| 35461 | caccaccagg catgccttac aagagctcct gaaggaagta ctaaataagg aaaggaaaaa |
| 35521 | ccggtaccag ccactgcaga aacataccaa attgtaaaga ccattgaaac tatgaagaaa |
| 35581 | ctgcatcaac taatgggcaa aataaccagc taacatcata atgacaggat caaattcaca |
| 35641 | cataacaata ttaaccttaa atataaatgg gctaaatgcc ccaattaaaa gaccacagac |
| 35701 | tggcaaattg gataaagagt caagacccat cagtgtgctg tgttctggag acccatctca |
| 35761 | catgcaaaga cacacatagg ctgaaaataa agggatggag gaagatctac caagcaaatg |
| 35821 | gaaagcaaaa aaaaagcagg ggttgcaatc ctagtctctg ataaaacaga ctttaaacca |
| 35881 | acaaagatca aaagagacaa agaaggccat tacataatga taaagggatc aattcaacaa |
| 35941 | gaagagctaa ctatcctaaa catatatgca cccaatacag gagcacccag attcataaag |
| 36001 | caagttctta gagacccaca aagagaccaa gactcccaca caataatagt gtgagacttt |
| 36061 | aacaccccaa tgtcaatatt aggtcaacga gacagaaaat taacaagcat attcaggatt |
| 36121 | tgaactcagc tctggaccca gtggaactaa tagacatcta cagaactctc caccccatat |
| 36181 | caacagaata tacattcttc tcagcaccac atcacactta ttctaaaatt gaccacataa |
| 36241 | ttggaagtaa aacactcctc agcaaatgca aaagaatgga aatcataaca aacagtctct |
| 36301 | cagaccaaag tgcaattaaa ttagaactca ggattaagaa actaactcaa aaccatacaa |
| 36361 | ctacagtgga aactgaacaa cctgctcctg aatgactact gagtaaataa caaaaagaag |
| 36421 | gcagaaataa atacattatt tgagaccaat gagaataaag atacaacata ccagaatctc |
| 36481 | tgggacacag ctaaaacagt gtttagggga aattcatagc aataaatgcc cacaggagaa |
| 36541 | agcaggaaag agctaaaatc aacactctaa catcacaatt aaaggaacta gagaagcaag |
| 36601 | agcaaacaca ttcaaaagct agcagaagac aagaaataac taagatcaga gcagaactga |
| 36661 | aggagattag agacacaaaa aacccttcaa aaaaatcagt gaatccagaa gctggttttt |
| 36721 | tgaaaagatt aacaaaatag atagaatgct agccagattg ataaagaaga aaagagagaa |
| 36781 | gaatcaaata gacgcaataa aagatgataa agaggatatc accactgatc ccacaaaaat |
| 36841 | acaatctacc atcagagaac actataaaca cctctatgca aataaactag aaaatctaga |
| 36901 | agaaatgaat aaattcctgg acacatacac cctcctaaga ctaaaggaag aagtcaaatt |
| 36961 | cctgaataga ccaataataa gttctgaaat cgaggcagta attaacagcc taccaaccaa |
| 37021 | aaaaagccca ggaccagacg gattcacagc tgaattctac cagaagtaca aagaagagct |
| 37081 | ggtaccattc cttctgaaac tattccaatc aatagaaaag gagggaatcc tccctaactc |
| 37141 | attttatgag tccggcatca tcctgataca aaaacctggc agagacacag caaaaaaata |
| 37201 | aaattgtagg ccaatatccc tgatgaacat tgatgcaaaa atcttcaata aaaaactggt |
| 37261 | aaactgaatc cagcagcaca tcaaaaagct tatctaccat gataatttgg cttcatccct |
| 37321 | gggatgcaag gctggttcaa catatgcaaa tcaataaaga taatccatca cataaagaga |
| 37381 | accaatgaca aaaaccacat gattatttca atagatgcag aaaaggcctt tcataaaatt |
| 37441 | caacagccct tcatgctaaa aactctcaat aaactaggta ttgatggaac atatctcaaa |
| 37501 | ataataagag ctatttatga gaaacccaca gccaatatca tactgaatgg gcaaaagctg |
| 37561 | gaagcattca tttgaaaacc ggcacaaaac aaggatgccc tctgtcacca ctcctattca |
| 37621 | acatagtatt ggacgttcta gccagggcaa tcaggcaata gaaagaaata aagcatattc |
| 37681 | aaataggaag agaggaagtc aaattgtctc tgtttgcaga tgacatgatt gtatatttag |
| 37741 | aaaaccccat catctcagcc caaaatctcc ttaagctgat aagcaacttc agcaaagtct |
| 37801 | caggatacaa aatcaatgtg caaaaatcac gagcattcct atacaccaat aatgacaaac |
| 37861 | agccaagtca tgagtgaact cccattcaca attgctacaa agagaataaa atgcctagga |
| 37921 | atacaactta caagggatgt gaaggacctc tttaaagaga actacaaacc actgctcaat |
| 37981 | gaaataagag aggacacaaa caaatggaag aacattccat tctcatggat aggaagaatc |
| 38041 | aatatcgtga aaatggccat actgcccaaa gtaatttata gatccaatgc tatccccatc |
| 38101 | aagctaccat tgactttctt catagaatta gaaaaaacta ctttaaattt catatggaac |
| 38161 | caaaaaacag cccgtatagc caagacaatc ctaagcaaaa tgaacaagct ggaggcatca |
| 38221 | tgctacctga cttcaaacta tactacaagg ctacagtaac caaaacatca tggtactggt |
| 38281 | acataaacag atagatagac caatggaaca gaacagaggc ctcagaaata acgccacaca |
| 38341 | tctacaacca tctgatcttt gacaaacatg acaaaaacaa gcaatgcaga aaggattccc |
| 38401 | tatttaataa atggtgtcgg gaaaactggc tagccatttg cagaaaactg aaactggacc |
| 38461 | ccttccttac acgttataca aaaattaact caagatggat taaagactta aacataaaac |
| 38521 | ataaaaccat aaaaacccta gaagaaaacc taggcaatac cattcaggac ataggcatgg |
| 38581 | caaagacttc atgactaaaa taccaaaagc aatggcaaca aaagccaaaa ttgacaaatg |
| 38641 | ggatctaatt aaactaaaga gcttctgcac agcaaaagaa actaacatca gagtgaacag |
| 38701 | gcaaccgaca gaatgggtga aattttttgc aacgtatcca tctgacaaaa ggctaatatc |
| 38761 | cagaatctac aaggaaccta aacaagttta caagaaaaaa aacaacccca tcaaaaagtg |
| 38821 | ggcgaagggt atgaacagat gcttctcaaa agaagaaatt tatgctgcca acaaacatac |
| 38881 | gaagaaaagc tcatcatcac tggtcattag agaaatgcaa atcaaaacca cagtgagata |
| 38941 | ccatcttatg ccagttagaa tggcgatcat taaaaagtca ggaaacaaca gatgcaggag |
| 39001 | aggatgtaga gaaataggaa cacttttaca ctgttggtgg gagtgtaaat tagttcaacc |
| 39061 | attgtggaag acagtgtggt gattcctcaa ggatctagaa ccagaaatat cttttgaccc |
| 39121 | agccatccca ttactgggta tatactcaaa ggattataaa tcatgctact ataaagacac |
| 39181 | atgcacatgt atgtttattg tggcactatt cacaatagca aagacttgga accaatccga |
| 39241 | atgcccatca atgatagact ggataaagaa aatgtgacac acatacacca tggaatacta |
| 39301 | tgcagccata aaaaaggatg agttcatgtc ctttgcaggg acatggatga agctggaaac |
| 39361 | catcattctt ggcaaggtaa cacaggaaca gaaaaccaaa caccacatgt tctcactcat |
| 39421 | aagtgggagt tgaacagtga gaacacatgg acactgggag gagaacatca cacactgggg |
| 39481 | cctgtcaggg tgtaggaggc taggggaggg atagcattag gagaaatacc taatgtagat |
| 39541 | gacaagttga tgagtgcagc aaaccaccat ggcatctgta tacctaggta acaaacctgc |
| 39601 | acgttctgca catgtacccc agaacttaaa agtattatta ttattattat aataataata |
| 39661 | ataaaagaaa tacaataaaa tagaatgcag catacagcag tgattctcaa acacattcag |
| 39721 | catcagaatt acccttgaat ctttaaaata tatatacata tgagatctta gtctccaaga |
| 39781 | tttgtaagtt tggtattggg tccctgggcc tatgttgggt ttagaaactt ctacagatgg |
| 39841 | tttggatgta tgggacagtt taagaatcgc tgaactaaaa tcaaataaac tgaatatcct |
| 39901 | gtgatttaga gagacttatc gtttatttca ctatccaagt acttgcatta gagcgtggct |
| 39961 | agaagggatt tgcagccttg taaataatca gaaattcaga cattttgaga tgagagaact |
| 40021 | gctgaagatt ttattctgac ttgaaataaa ttttctaatt agaaacttcc aggtgagagc |
| 40081 | aaaggcctgg aacaatattc ctgagccaga ggaggatcga gtttgactcc aggcctaaca |
| 40141 | cttactaggt ctatgacctt gggtcagtaa tttaaattct ctgtatctca acctctcaac |
| 40201 | agggtattgg tagggattaa atgtgttagt gtctgtgaag tgcttagagc agtgcttggc |
| 40261 | atagtaaatg cttaatgaat ttcagccact gtttttattt ttagtacttt ccagctcccc |
| 40321 | caaaaagata ctttttttag acttgtatta agacaataaa aagtttaatc agcatgcttc |
| 40381 | atacctaaat atgcttcact ttatagcaaa gtttacaaga ctaaaactgt tttgttgtaa |
| 40441 | ttctctgagt ctcatgtgtt tattaatgat tttttctgct gtttattcat ctgaattcta |
| 40501 | ctcattcttc aagacctagc tggaatcctg tttctagaaa gactcttgcc cataataata |
| 40561 | aacctgccct atctgagttc ctaggtggtc tgtacctcat aatttggtaa ttaattgtat |
| 40621 | atgcacttat ataacaaaac attattgtgt gtctttgctg tatcagattc taggctggaa |
| 40681 | gttgtagata tgatgttttt gtctagaaaa atgttctaga atgtcctact caggacagtc |
| 40741 | tgttgacttt aaagacacat ttcctaaaca gacacttcat gaggcagccc cagcctgtac |
| 40801 | ctgtgttcct ggacctgatg atcaagtttg atttaagcct caccacttac tagctctgtg |
| 40861 | attttgggca agttacttga attctctgtg tgtagataga acaatgttga gggaaatccc |
| 40921 | tttcccccat ccttgtgttt ccacaaggga acttgcttcc taataagtaa cactttcagg |
| 40981 | ggaatattct aggcccttct cttatcccca ttacttgttc tttctgtgaa aagaggagag |
| 41041 | gttaatctga tggatgaaat ccttaatctt tcatcttctg gactgtagag cctgtgaacc |
| 41101 | aaagcaatgg accacttgca ctgaaattga ggctgaccct gtattttgat tcttatttgg |
| 41161 | caacttattt ctattctgtt cccaattcaa aatcccaagg ggagaaggaa gataattgat |
| 41221 | taccagaagt atgtaatggt ggtaggaagt tgaataaatg gtaacttttt aaaagttgca |
| 41281 | tgagatatag tccttatccc agagaagcta agtttgcttt tctttcctct catgtatttt |
| 41341 | agtattattt ctacaattag attgtaaacc ctttaaaagc aagaatattt ctacattttc |
| 41401 | ttactcctga tagcacacag tagactgctg ggcacataca tagtaggtgg ctctgtaggt |
| 41461 | acttgctaaa tgattcaaca tgtttttccc tcatggaaaa gaaagatttc agtattgttc |
| 41521 | ttatcagcta ggaaggcact ctgaatagga aatcagttct aggcaggtat ccataaatgg |
| 41581 | gttatgattt ccaacttact tgccccagag gctcgctaat gttgaactct tcatgggtac |
| 41641 | tttgtcttgc ttcatgagct atacatgcta aggggttagc agatcatata atcttttgat |
| 41701 | ctacaaaata tgatctttat tgaacaaaaa cttgggccaa aggcctttct cctttgccac |
| 41761 | cttcctccct cttttcattc tcttttttgg gaatgccctt tgtgcatgtt agttacagca |
| 41821 | tgtaccacat tgcactgtat tgttggtttt tgggtctaac ccacccttaa cactgcagtc |
| 41881 | cccaagggca gaaattcagt ctcattcatt ttgatgtcct cagtgcctgt gctcagagaa |
| 41941 | tatctattat ttgaaaaaat agtgcaaaag taaattttag gagactacat cacactcatc |
| 42001 | taaactgcaa gtttgacaag ttgacatcca aaagaaaggc tctcctaaat aacctcgcca |
| 42061 | cagaaatttg ggtgaccttt gtagctctgg agaaagcaga ggcaaaaatg aaacctaaaa |
| 42121 | attatttgtg ggtttttaaa aaatgttttc tcatggagta aaggtctaca gctgagttct |
| 42181 | tttcatatga gggaatgaca gaaacacagc tggttctgac tttcagcttc aactgagcga |
| 42241 | ccagagctct gctggtgaaa caggaacttg tattgtgccc ctgacgtgca ccttgaaggt |
| 42301 | gtcagctcat tgtccctttg ttcacataaa tagtttttta agaattgttt ttgatcttgt |
| 42361 | gagcctctaa ctaaatgatt aaccatgcaa agttggccat ttggggtaat actgaagcac |
| 42421 | ttctcttgag ggctattgac aggtgggaat gtgcccacct ccttgggtct ctggttttca |
| 42481 | tgtcatactt gcaaatcagt gacagtttaa acttggggca atcacttagc aagtctattg |
| 42541 | agttaccaag ttaattattc ccactttgca tgaagcaacc ttgaaaatga ttttcctaaa |
| 42601 | gcaaagtaca tccaaactca gtaccttctt aataaccttt gctgaatgaa taaatgacta |
| 42661 | attcataaaa aatgtaacat atctttaatt cttacttacg ggcagtttaa gcctcttgtg |
| 42721 | taagaggagg cctcggcttg agataacata ggatagtaag cctcctagag aaatttctat |
| 42781 | atggaaacat ggtctgctat gaagctagaa gtgagaggac attatatttg accattatat |
| 42841 | ttggcttcag agcttctcaa catggggccc aaagtcaagg tcccttgttt cattaagagg |
| 42901 | aggtccagga gtgcatgaca cccatcagac tactgagacc cagctggaac taggcacctt |
| 42961 | gcacaggggc cttgcctaat caaaatagtt cttatttttt ctgagttcca agtaactagt |
| 43021 | ttcctaaccc agtgtctgga tagtagtgcc aagtgggagt accttcaatg aacttcctca |
| 43081 | tgaggttatt tctagcctat tggaatgttt cgttttagga gggtgaggaa gggaagtctt |
| 43141 | gaatttttgt gcttagttta atgttgtgat acagctttga ccatccgttt aatgggagat |
| 43201 | ctgttttcca gatgactata catgtggaaa ggagaagttt tttgagtgtt ttttttaacc |
| 43261 | ccttttaaag aatggttttt catttagtct ctacatttgg gggtaaaagg tcctctaggg |
| 43321 | agacttttca aaagtatttg aagtttgcat ctgatttcag aggtgagttg gaggcctatc |
| 43381 | tgtgtatgac agacacatgt ctccaacaac tatatgttca caaggactaa gagccatcct |
| 43441 | tttgggtcca tcattcaaca ttgatctcac attcgtgttc gtatcagtat ctttacagtg |
| 43501 | cgctcccagt tacatctccc taatttccct tagtaggctt cacagaattt gcagtgtatg |
| 43561 | caatggcaga tgaccacatg tggagtcatt taaccacatc ttccactgca agtcagcccg |
| 43621 | ctcttgatgt ctgtttatgt ttagattcca tcttttggaa gatttcattc ctctgcacta |
| 43681 | tctcagtatc tcagatgctt ttgagactgg gtccttttcc cctcctatgt ttggccatgg |
| 43741 | ccaccccctc agggttgtgt tgtgtttcac agctgctgtt tgtagggttg acctttacaa |
| 43801 | tgtacaaagc tctttcccat atgttgacaa tccctggtgt gatgctgtga gttaggcagg |
| 43861 | gtgtgtatac gtgtcctcat catattacag tggtaaggca acagggtttt tgaatttgat |
| 43921 | cacccatgaa tttgtctaat ttgttggtaa aaaatggtca tgtatcagcc gtttcacagg |
| 43981 | gtcagcttaa tagaaagtgg gagttaggca ggaccagaat tcaggacttc agcccccggt |
| 44041 | cccagggact attctctata cccaattgtc ccaccttgaa tcagtttctt ctagggaaat |
| 44101 | atctccaaaa ctgagatggc acccacagga cttcttaatt gtagtcatta ccaggaaaaa |
| 44161 | caagcaaagg aactggtgta aatctctgtt tttggtgatt ggtggagatt tggagattgt |
| 44221 | cttgtgtcaa aagtaaagcc actagattaa atgttttgtt aataaattgg ttatttttaa |
| 44281 | tttaattatt tgacagttaa tttacattat tcaaaaatca aaataaaatt taaaagaagt |
| 44341 | ttacactgaa aagtcttgcc ccacttatac cctgctcacc tcagtatccc ccaatacata |
| 44401 | ccatctataa ggtgatcatt tgtattagtt tcttgtgaat ccttgatagt gtgttttata |
| 44461 | tagatacagg taaatatgag tatgtactat tatttccccc ccaccccacc ctgttttttt |
| 44521 | tttgagacgg agtctcgctc tgtcgcccag gctggagtgc agtggcacga tctcggctca |
| 44581 | ccgcaagctc caccattttc ccccattttt aaaacaaaag gtagtagcca tatatacact |
| 44641 | attttacacc ttgttttatc acttactaat atataccaga gagctttcca tcattttgta |
| 44701 | catatgcacc tatatctgtc aattattccc agaagtggaa ttgctgggtc agcaggaaaa |
| 44761 | atcatgtata attttgatag gtattgccta attgtcctgc acagggcttg aattgtttgt |
| 44821 | actcccacct ttagtgtatg agaagacctg tttctccata gcctcatcaa acagagtgtg |
| 44881 | tgagattaga tgagaaatag gaggtgagca gtcttttacc ccatccgtag tttgcagtgg |
| 44941 | gaacactgca cagttgcaag agctggtgca ggtatcagat tagttccagt ggaaacgctg |
| 45001 | cctcaccatg gccatgggct tgcgccagct ctagtgacac acacggaatg gacccacgtt |
| 45061 | gccacttgca gaatttcctg tagcagaaag ttgaacatgc attcattatt catctaacta |
| 45121 | gccatgctgg atctaaagag cacaacagtg ttttttagaa ccaaaaagaa aattgtttca |
| 45181 | ctacaacaca ctgtgtataa ggctttcaat gctcttttct cagctattaa cattattttc |
| 45241 | aggactgagt tcaagagatg tatcccaaat cacagggatg tcttgctaag cttggaactt |
| 45301 | tcatactcaa gggatgcttt tttgaggaat gattttacac ttactcaaca tttgtaatta |
| 45361 | aataattagt actttataag ataaatttaa actgtccaag tacaatataa acattgaact |
| 45421 | atgatgcatt attgctagac tttttcctta aagttgccaa gtggtttcct gcattaggca |
| 45481 | aataggggat catataaaaa tgccatgatt tacggcctag ataacatctc caccatttga |
| 45541 | gcagcatata ttccaggtca tccccacata actccttacc attctcatta gaaaggttga |
| 45601 | ttcttagtct tatttttctc tgaggacagc aaaaaaaaaa atccccttca gttccactgc |
| 45661 | atagaaaagt gtggtaaatg gagccgggca cagtggttat ttaatttaaa tggacaatat |
| 45721 | tttttataga attttgacag ggccactgta taggggaaag tcactcctct tcccctttat |
| 45781 | agaagagttg cacctggaca gttgcattga tgactgtatc cagtctacac aagaggtcat |
| 45841 | tcctgggcat aagaatggac tgccaaaatc tagctgaaac accattgaca aatagacatt |
| 45901 | ttcttttgtt aataatacct gtgaaggctt tcataacaga catttccagt tttgttctca |
| 45961 | ggctccttgc agctgctcct ctaaaagtgt gctctcttcc aagagctgac aatggccaga |
| 46021 | agcaaggtgt tctgtctttt gtgccatcat catctaactt gccacacaca tttgggatgt |
| 46081 | cagcctaggt ataggttttg tatccactca gtatggcttg tgggtctggt tgcctttgtt |
| 46141 | attcatgctg agggcctctg ggcatcagtt tggtgtgaga gaacccattc catgaccctc |
| 46201 | cttcctttgg ctgttttgac tcgatggctc ttgttggcac agtctgtgag tgtctgatgc |
| 46261 | tctatccatg ccggaccatc tgttctgctg tctctgtggt ctgaagtcgt tttctgaact |
| 46321 | attccttgat aataaatttg agatgatctt gttctacctt tcttttcaag tcacatctta |
| 46381 | gccccttagc cacattcccg aagaacatga caaatggatg ggtcacaagt cacgtagcat |
| 46441 | agggtgtcag accacgaggc tttgaaggga ttctgttggg tgctaaaaag aaagattttg |
| 46501 | tgtcaccacg atttttttta aaggcatgtt gacacttagg ccttaattga aagcgttctt |
| 46561 | actcaagtag agttgacaga ggagtatttg gtagtcgcgg ttgctggtct gaagagcatg |
| 46621 | tggttctgtt tcaatgccca atgagatctt ctcacgggaa aatgttctga catctcaaac |
| 46681 | aaatgacctt catgcatagt tttgacaaaa taccctatta agtatgcata tatggttggt |
| 46741 | accttgtggt aataattcaa tactggaaac agagtagcaa caaagaaaca ttagggttat |
| 46801 | atttaacctc tgtggaatta gtgtgtaaac aaactgctta tcagaaatgc tcatatgggg |
| 46861 | ctttgtttaa ataaataaga aactggcata tagggtctgc aggatatttc tgccaagtag |
| 46921 | acctccctca cattataaga caccacatct atgtctgacc ccatatggaa agaggcatag |
| 46981 | caagccagca ctggttcata ttccctctcc accacataat gggtatgtga tcttagggaa |
| 47041 | tccaccgaaa ctctctgggc ctcagtttcc tcagctataa atggtggata atcaaattat |
| 47101 | ttacctcacc attaataaat gttagctatt attttttatc aagtttaata caaagagaaa |
| 47161 | cattttactt atttttccag ctatccagag catcttccaa aatcctatca ccaacaaata |
| 47221 | ctgtattgta tttattatag caactatgta aaaatggagt ccctgtccta tgcttagatg |
| 47281 | aaatatgttg gtatttgagt ttgcatgtct tctataggaa tcagtgttta gtgaaaacgg |
| 47341 | gtggagataa acagatgttt tcacagtcct gttgttcaca gtaccgccaa attgaatgtt |
| 47401 | tccatatagg tgcattctaa tggcttaaat gatgcagata ttttctggcc agccatatgg |
| 47461 | atcttttgtc atctaagatg ttaatatttt ccttatattt tatagtagtt ctggagtaca |
| 47521 | gccagtttct tgaatagggt ccacatggct cattatgcac agggcctgga aactgcctta |
| 47581 | ctcgtgctgt tgaaatgaac cgtgacactt cagaagagct gggagctggg gtagagcagt |
| 47641 | ggctaggaga acatattcaa ttatatttcc tcctgcatta agctacaagt aatgagcact |
| 47701 | ttcctgtgct ttacagttaa gtaattaaaa gaaattatag agtgggatgc aaaaataacc |
| 47761 | cgaaggacaa ctggatgtgt ggagccacca gttttctcca tgagtgcaca aggttaatcc |
| 47821 | ttgttactac tcagaatgct gagtttctac agaaagggtt gcaggtccac acatgttttg |
| 47881 | gcgtctaccc acacgcttct gtatggcatg actgtgcatc ccagaagaag ggctgtgctg |
| 47941 | tgtacctcca cgtttcagtg gaatttaaca aactgatccc tgaaaatggt ttcataaagg |
| 48001 | tgagtaacag agagctaata gccttctctt gctaatttta tctttccccc aagatttctt |
| 48061 | gataatagtt tgaaaaggag tgttattctt tggtctctag aggcaactta cctttccagt |
| 48121 | ttcttccatc acctgttttc atctctcttg tttttttaaa tttaatgctg tatgtatttc |
| 48181 | agaggatagg atctaatcta gtgcggtccc ttcatcaggt gagaattatt catctcattt |
| 48241 | tcattttagc ccttctgaat taatgacatt gaagcccggc agtttggtcc taagatgggt |
| 48301 | ttaattatgt acagatactc tttctataat ggaaattgct cagataacta attaaccaca |
| 48361 | agaatacact gtctatggaa aatttcagga gcaccgtctg tggaaaaact gggaagggca |
| 48421 | tgctgtcacc acagctctgg ggtctattaa aagtgtggtt atgcagcact ggtgtctagt |
| 48481 | ggggtgttgg ctctcaactg ccagaattcc catagcattt catggcagaa agtcaaggtg |
| 48541 | tccagcaata ctctgaaagt gacctgttga ttaaagtcgt caattctgaa gaaagagact |
| 48601 | gaaataagac aaatgggtct taactttttt tctctttctc tctcttgtaa aaatgtgtga |
| 48661 | ttgttctggc atgttcccaa tccccacata atgccaacat cttttcttaa agggggattc |
| 48721 | cctttatcct tggatctgag aattattgca tgttctccct ttagggacaa tgaatgcagt |
| 48781 | tgcatcaccc ttgctttttt tttttttttt gtacacagca tgcttattct tggatgcagg |
| 48841 | gacttgaaag acaaagcccc acctggcttt cacaacatct cctattagta ggtgtgcctt |
| 48901 | gtgtgtaatt tgaaggaggc ggtcccttag ctgtgtttac actgtacttt taaatgtggg |
| 48961 | gctgaaggta gaatcaacca tacttaagat gccacctggg aaaatagggt tctgtgtcat |
| 49021 | ctcagcccca cccatttgca aatgacttaa cagcagcact attagggttc ctagtgtgag |
| 49081 | tcatttgcat ttggactggt gaacttggtg acttcttggt gtttggaaac aaacaacctt |
| 49141 | tgcagtcttt cgtaaaaagc ctgaacagtg gaccagtctc cagttctact tgcaaagctg |
| 49201 | cccccatcaa atccctcata atgttcaact taaaaaatgt tacacttttc tctggaaatc |
| 49261 | taaccttttt tcctttttta aaagccattt taagtacttc agtcttgaat caaatgatcc |
| 49321 | caaatattgg acaccaacct agaaattggg ttacctcctg ggaactttat cgaagaagag |
| 49381 | agattttggt tggagagggg gttttgatgt ttgatactta tatttactat tttaatattt |
| 49441 | cattgttgtt gttgctgctg ctgctgtatt attttgcgag tttcgtttgt ttaaatttca |
| 49501 | tggtatttgg taggagagag ctggatctgt tggtttcagg acaagtctag aaataagaaa |
| 49561 | tctgccttga gtgagtgagt tggttccctc tgttgctatt tcaccattaa ggacgaaagg |
| 49621 | aactcacaag gaccagagac atctggctga aagcaatact agtgtgactg gacatctact |
| 49681 | acctgccata gttggtcata tcgtttccag tatgattctg attgagtgag tgatattagg |
| 49741 | ctatgttcag ggatcaggga ggctaattat gcttatattg ccttgtagca ttttggtaag |
| 49801 | aattaatgat tgtgtagatg tccagattta ggtcagcaat attctaaaag ttctcattga |
| 49861 | actaatcatg tttataagta gcctgtactt tctatcataa taacaatagt ggaaaagcta |
| 49921 | gttgacataa aaggagccca gattttactt aagtaaaaac acaaaagcaa agatattttc |
| 49981 | ccacataaat tacaaaagca aagatatttt cccacataaa tgtccccata aaacaagttg |
| 50041 | aaccaaagag gaaagatgac aggtaaccgt atgacacgct aagaaagtat cataatactt |
| 50101 | aagttaactt caacctttta tttccttatc ctaagcagcc tcttttctct ttatcattta |
| 50161 | gtcctgtgct tctcaacttt gataagtaaa aaagttattg cactaaataa atettattga |
| 50221 | aatgcaggat ctgattgagt gggtggggta ggtggaatga gggtggggaa gttgagattc |
| 50281 | tgcatttctt agaagtttct actttatgtt aaaatggcta atccatctca acattgagaa |
| 50341 | gtaaggtttc acttaatttc agcctgtgta agtttatccc atatgtacat ttcctaaaac |
| 50401 | tctaatctca ggccccagga atttctcctt tagttaaaat atttttagga ataaatttga |
| 50461 | attgcattaa tacacaattt ataaatttaa cacaaaaaat tatttgaagt ttgagacttt |
| 50521 | aggttgcatg aaatcaattt catacttgaa aattttctat aaattcaaaa gtctgtgtat |
| 50581 | ttaaatacaa tttaaatacc tgtgttacag tgacatttgt ttttctgtct ctctctccac |
| 50641 | catttccaga gtcatcatcc ctgtacagaa aaatttttcc cacatgattt caccataaat |
| 50701 | tcattaaata tgatgcttac ttgataattt ctccaggttc tttttttttt taattatact |
| 50761 | ttaagttcta gggtacatgt gcacaacctg caggtttgtt acatatgtat acatgtgcca |
| 50821 | tgttggtgtg ctgcacccat taactcgtca tttacattag gtatatctcc taatgctatc |
| 50881 | cctcccccct acccctactc catgacaggt cccagtgtgt gatgttcccc accctgtgtc |
| 50941 | caagtgttct cattgttcag ttcccaccta tgagtgagaa catgcggtgt ttggttttct |
| 51001 | gtccttgcga tagtttgctc agaatgatgt ccttgctcac tgatggacat ttggttggct |
| 51061 | ccaagtattt gctattgtaa atagtgccgc aataaacata cgtgtgcatg tgtctttata |
| 51121 | gtagcatgat ttataatcct ctgggtatat acccagtaat gggatggctg gctcaaatgg |
| 51181 | tatttctagt tctagatcct agaggaatcg ccacactgtc ttccacaatg tttgaactag |
| 51241 | tttacagtcc catcaacagt gtaaaagtgt tcctatttct ctacatcctt tccagcacct |
| 51301 | gttgtttccg gactttaatg atcgccattc taactggtgt gagatggtat ctcattgtgg |
| 51361 | ttttgatttg catttctctg atggccagtg atgatgagca ttttttcatg tgtcttttgg |
| 51421 | ctacataaat gtcttctttt gagaagtgtc tgttcatatc cttcacccac tttttgatgg |
| 51481 | ggtcatttga ttttttcttg taaatttgtt taagttcttt tagattctgg atattagccc |
| 51541 | tttgtcagat gggtagattg taaaaatttt ctcccattcc gtaggtttcc tattcactct |
| 51601 | gatggtagtt tcttttgctg tgcagaagct ctttagttta attagatccc atttgtcaat |
| 51661 | tttggctttt gttgccattg cttttggtgt tttagtcatg aagtccttgt ccatgcctat |
| 51721 | gtcctgaatg gtattgccta ggttttcttc tagggttttt atggttttag gtctaacgtg |
| 51781 | taagtcttta attcatcttg aattaatttt tgtataaggt gtaaagaagg gatccagttt |
| 51841 | cagctttcta catatggcta gccagttttc ccagcaccat ttattaaata gagaatcctt |
| 51901 | tccccatttc ttgtttttgt caggtttgtc aaagatcaga tggttgtaga tgtgtggtat |
| 51961 | tgtttctgag ggctctgttc tgttccattg gtctatatct ctgttttggt accagtacca |
| 52021 | tgctgttttg gttactgtag ccttgtaata tagtttgaag tcaggtagcg tgatgcctcc |
| 52081 | agctttgttc ttttggttta ggattgtctt ggcgatgcgg gctctttttt ggttccatat |
| 52141 | gaactttaaa gtagtttttt tccaattctg tggagaaagt cattggtagc ttgatgggga |
| 52201 | tggcattgaa tctataaatt accttgggta gtatggccat tttcatgata ttgattcttc |
| 52261 | ctacccatga gaatggaatg ttcttccatt tgtttgcgtc ctcttttatt tccttgagca |
| 52321 | gtggtttgta gttctccttg aagaggtctt ccacatccct tgtaagttgg attcctaagt |
| 52381 | attttattct ctttgaaaca attgtgaatg ggagttcact catgatttgg ctctctgttt |
| 52441 | gtctgttatt ggtgtatagg aatgcttgtg atttttgcac attgattttg tatcctgaga |
| 52501 | ctttgctgaa gttgcttatc agcttaagga gattttgggc tgagatgatg gggttttcta |
| 52561 | aatatacaat catgtcatct gcaaacagag acaatttgac ttcctctctt cctatttgaa |
| 52621 | tatcctttat ttctttctat tgcctgattg ccctggctag aacgtccaat actatgttga |
| 52681 | ataggagtgg tgacagagga catccttgtt ttgtgccagt tttcaaaggg aatgcttcca |
| 52741 | gcttttgccc attcagtatg acattggctg tgggtttgtc gtgaatagct ettattattt |
| 52801 | tgagatatgt cccatcaata cctagtttat ttagagtttt tagcacaaag gctgttgaat |
| 52861 | tttgtcaaag gccttttctg catctattga gataatcatg gtttttgtct ttgattctgt |
| 52921 | ttatatgatg gattatattt attgatttgc atatgttgaa ccagccttgc atcccaggga |
| 52981 | tgaagccaac ttgatcatgg tggataagct ttttgatgtt ctgctggatt cggtttgcca |
| 53041 | gtattttact gaggattttt ccatcgatct tcatcaggga tattggcctg aaattctctt |
| 53101 | tttttgttgt gtctctgtca ggctgtggta tcaggatgat gctggcctca taaaatgagt |
| 53161 | tagggaggat tccctctttt tctattgatt agaatagttt cagaatggta ccagctcctc |
| 53221 | cttatacctc tggtagaatt cagctgtgaa tccatctggt cctgatggat ttttttggtt |
| 53281 | ggtaggctat taattattgc ctcaatttca gagcctgtta ttggtctatt aagagattca |
| 53341 | acttcttcct ggtttagtcc tgggagggtg tgtgtgtcca ggaatttata aatttctttt |
| 53401 | aggttttcta gtttatttgc atagaagtgt ttatagtgtt ctctgatggt agtttgtatt |
| 53461 | tctgtgggat tggtggtgat atccccttta tcacctttta ttgcatctat ttgattcttt |
| 53521 | tctcttttct tctttattag tcttgctagt gatctatcaa ttttgttgat ctttttaaaa |
| 53581 | aaccagctcc tgggttcatt gattttttga aggagttttt ctgtctctat ctccttcagt |
| 53641 | tctactctga tcttagttat ttcttgtctt ctgctagctt ttgaatgtgt ttgctcttgc |
| 53701 | ttctctaaat tgtgatgtta gggtgtcaat tttagatctt tcctgctttc tcttgtgggc |
| 53761 | atttagtgct ataaatttcc ctctacacac tgctttaaat gtgtcccaga gattctggta |
| 53821 | tgttgtgtct ttgttctcat tggtttcaaa gaacatcttt atttctgcct tcacttcgtt |
| 53881 | aagtacccag tagtcactca ggagcaggtt gctcagtttc catgtagttg agtggttctg |
| 53941 | agtgagtttc ttaatcctga gttctagttt gaaagcactg tagtctgaga ggcagtttgt |
| 54001 | tataatttct gttcttttac atttgctgag gagtgcttta cttccaacta tgtagtcaat |
| 54061 | ttttggaata agtgtgatgt ggtgccgaga agaatgtata ttctgttgat ttggagtgga |
| 54121 | gagttctgta gatgtctatt aggtccgctt ggtgcagagc tgagttcaat ttctggatat |
| 54181 | ctttgttaat tttctgtctt gttgatctgt ctaatattga ccgtggggtg ataaagtctc |
| 54241 | ccattattat tgtgtgggag tctaagtctc tttgtaggtc tctaaggact tgctttgtga |
| 54301 | atctggtgct cctgtattag gtgcatatat ttttaggata gttagctctt cttgttgaat |
| 54361 | tgatcccttt atcattatgt aatggccttc tttgtctctt ttgatctttg ttggtttaaa |
| 54421 | gtctgtttta tcagagacta ggattgcaac tcctgctttt ttttgctttc catttccttg |
| 54481 | gtagatcttc ctccatccct ttattttgag cctatgtgcg tctctgcaca tgagatgggt |
| 54541 | ctgctgaata cagcacactg atgggtcttg actctttatc caatttgcca gtccatgtct |
| 54601 | tttaactgga gcatttagcc catttacatt taaggttaat attgttatgt gtgaatttga |
| 54661 | tcctgtcatt atgatgttag ctggttattt tgctcgttag ttgatgcagt ttcttcctag |
| 54721 | cctcaatgat ctttacaatt tggcatgttt ttgcagtggc tggtactggt tgttcctttc |
| 54781 | catgtttagt gcttccttca ggagctcttg taaggcaggc ctggtggtga caaaatctct |
| 54841 | cagcatttgc ttgtctgtaa aggattttat ttctccttca cttatgaagc ttagtttggc |
| 54901 | tggatatgaa attctgggtt gaaaattctt ttctttaaga atgttgaata ttggccccca |
| 54961 | ctctcttctg gcttgtagag tttctgccga aagatgctgt tagtctgatg gacttccctt |
| 55021 | tgtgggtaac ctgccctttc tctctcgctg cacttaatgt tttttccttc atttcaactt |
| 55081 | tggtgaatct gacaattatg tgtctttgag ttactcttct tgaggagtat ctttgcggca |
| 55141 | ttctctgtat ttcctgaatt tgaatgctgg cctgcctcac tagattgggg aagttctcct |
| 55201 | ggataatatc ctgcagagcg ttttccaact tggttccatt ctccccatca ctttcaggta |
| 55261 | caccaatcag atgtagattt ggtcttttca catagtccca tatttcttgg aggctttgtt |
| 55321 | catttctttt tactcttttt tctctaaact tctcttcttg cttcatttca ttcatttgat |
| 55381 | cttcaatccc tttcttccac ttgattgaat cagctactga agcttgtgca tgtgtcacat |
| 55441 | agttctcgtg ccatggtttt cagctccatc aggtcattta aggtcttctc tatgctgttt |
| 55501 | tttctagtta gccattcgtc taatgttttt tcaaggtttt tagcttcttt gctaaaaggt |
| 55561 | tcaaacatcc tcctttagct cggaggagtt tgttattact gatcatctga agccttcttc |
| 55621 | tctcaacttg tgaaagtcat tctctgtcca gctttgttcc attgctggcg aggagctgca |
| 55681 | ttcctttgga ggagaagacg tgctctgatt tttagaattt tcagcttctc tgctctggtt |
| 55741 | tctccccatc ttattggttt tatctacctt tggtctttga tgatggtgac gtacagatgg |
| 55801 | ggttttggtg tggatgttct ttctctttgt tagttttcct tctaacagtc aggaccctca |
| 55861 | gctgcaggtc tgttggagtt tgctggaggt ccactccaga ccctgtttgc ttgggtatca |
| 55921 | ccagcagagg ctgcagaaca gcaaatattg cagaacggca aatgttgctc cctgattgtt |
| 55981 | cctctggaag cttcgtctca gaggggcacc tggccgtatg aggtgtcagt cggcccctac |
| 56041 | tgggaggtgc ctcccagtta ggctactcag gggtcaggaa cccacttgaa gaggcagact |
| 56101 | gtccattctc agatatcata ttccatgctg ggaggacccc tactcttttc aaagctgtca |
| 56161 | gacagggaca tttaagtctg cagaagtttc tgctgtcttt tgttcagctg tgccctgccc |
| 56221 | ctagaggtgg agtctacaga ggcaggcagg cctccttgag ctgcggtggg ctccacccat |
| 56281 | ttcgagcttc ctggctgctt tgtttaccta ctcaagtctc agcaatggtg gacacccctc |
| 56341 | ccccagcctc gctgctgctt tgcagttcga tctcagactg ctgtgctagc agtgagccag |
| 56401 | gctccgtggg catgggaccc tccgagccag gcctgggaca taatctcctg gtgtgccgtt |
| 56461 | tgctaagacc attggaaaag cacagtatta gggtggggag tgtcctgatt ttccaggtac |
| 56521 | cgtcagtcat ggcttccctt ggctaggaaa gggaattccc caaccccttg tgcttcctgg |
| 56581 | gtgaggtgat gccccaccct gctttggctc atgctccgtg ggttgtaccc actgtctgac |
| 56641 | aagccccagt gagatgaacc cggtacctca gttggaaatg cagaaatcac ccgtcttctg |
| 56701 | catcactcac gctgggggct gtagactgga gctgttcata tttggccatc ttggaacctc |
| 56761 | cctttccaag ttctttatta cagagtgggt cactgaaact tcatggaaca aattggaaat |
| 56821 | tatcttctta attaatgtca ctgtctacca tgtatgggaa tttggtaaat attatatggt |
| 56881 | ttcaataaca tagtagatag aacattgtca aatctaaact tcagtgaatt gtaacagatc |
| 56941 | ccacctgaaa ttctaaagaa aacagaattc taattgaaga ggttaaactt ttacagggaa |
| 57001 | tgtcaactgc catttgggtc ctgtaaacaa aaaactgttt tttaaaaaag taaactttaa |
| 57061 | aagtattttc agatgacctc atttgctatc caagtggctt gagtatgctt gatgctaaga |
| 57121 | cttctttgtt acagactgga gatgtgtgct actggggcag tgttgctctg tgacaaggag |
| 57181 | gcagaggatg agggcaaggt tcgatgtgac tgtgaattct gggtggctct ggctatcggg |
| 57241 | agccttcatt gattacagca aaacagttgc tttcctaggg caatagtgtc tctgtcaccc |
| 57301 | aggctggagt tcagtggcat gatcaatcgc tcactgtagc ctcaacttct tagactcaag |
| 57361 | taatcctccc acctcagcct cccaagtagc tgaaactaca ggtgtgcacc accacaccta |
| 57421 | atttttttaa tttttaagtt tttgtagaga catggtctca ctgtgttgcc caggttgatc |
| 57481 | tcgaattcct gggctctagt gatcctcccg cctcggcctc ccaaagtgtt gggattacaa |
| 57541 | atgtgagcca ctgcacctgg ccctttgcaa ccttcttgac aatgcattcc tttattccct |
| 57601 | aactggaagt aacttctttc tctttataaa attgtatctg taccttttct gggtcatttc |
| 57661 | tacctttata ttctagttac gtatgtccta cctccctcct agggagggag gtaagtaaga |
| 57721 | ctggaaagta gacttcatgt gtgatgaatg aatgaacaaa aggaagtcta acatatggat |
| 57781 | atagtcaact ggatgcaaat taaaaatttt taaatattga tttgcaagat ttcattaagg |
| 57841 | tcaactctta atagtttgta tcatatatgt taggaaccaa atattaataa cttcttcagc |
| 57901 | attaccatta tctttatagg actgtctaaa atgagcagcc atatctttaa actgtgtttt |
| 57961 | ctctgattac acgctcacag gtaaaaccca aaggggctgg gaacaaacaa gacttttttt |
| 58021 | tttttctgta tgcctgaatt atctgtactg ttgcttgttt tcccaccttt ggccatagaa |
| 58081 | acttagttct aacatgctac aatttttgca gttctttctc ttagaaaaag accacattgt |
| 58141 | ctgaaatttc atccatttaa gtaatcaagc cttaaagttg aaggatcttg gtcatgatta |
| 58201 | atctagacct acaaagtagt atcttaatgg cactcctttt agaaagttag gttccaggac |
| 58261 | acacatagct gcagtgtcca cattttgtaa gctccttcgt tgtcacagcc actctcttct |
| 58321 | ctgtggctga tattctaaaa ctggcaacac atcctgatgg taaaagcttg gttcaggaga |
| 58381 | caggtgacct actagcttta tggcatttga caggttacct aacctctctg acgcataatt |
| 58441 | gcctcatcta tataatgggg ataataatac ccatcctgtc tccttgtaaa aatcaaatta |
| 58501 | gatgacgcct gtgaatgttc tatagtctct tagacaaatg taagttatga ctacagcaag |
| 58561 | agtaaaagag catgttgtta tggacattct ttcagtgaaa tgtctaagac ttgtgagtca |
| 58621 | cacttaaagc taaacttgat atctacttca ttgattttct ttttagttct atgtactata |
| 58681 | ttgaatttcc tgacagtggg gctatgaaag ccttcctagc attttataga tgtggttgaa |
| 58741 | ttaatggctg taagccttaa agcagaatta gacagcatca atgaatttat taagtataaa |
| 58801 | taaatatata atctgcttag caatattaca cagcctcttt atcttatgtg tgataaagag |
| 58861 | tcatccgaag gttgaaaatg aagaattgtc ctggaagctc ttacttaatc ttttattatt |
| 58921 | tcctaataca gtatataaaa ttactcattg aaagcttagc agaataagaa acaagaagtt |
| 58981 | aaaaggctga aaactacaaa ttttgctatt attattgtta ttacttccca agtctcttat |
| 59041 | tgatctgtta gaaatagagc tacacaggaa attgtaggac agttagtatg tggtagtgtt |
| 59101 | atctgctttt taattattca agtaaggttt tattccatta gaggaactca agaagttggt |
| 59161 | catggctgat aattgctatc tgtcaaattc cttagagcag ggatccgcaa cacccaggcc |
| 59221 | atggattgtt accagtccct ggcctgttag gaaccaggct gcacagtagg aagtgagcgg |
| 59281 | cgggtgagca aacattgcca tctgagctct gtctcctttc agatcagcag cagcattaga |
| 59341 | ttctcataga agcatgagcc ctgttgtgaa ctgcacatgc aagggatcta ggttgtgtgc |
| 59401 | tccttatgag aatccaattc cttatgataa tttaactgat gatctaaggt ggaaccattt |
| 59461 | catcccaaaa ccatccaccc tgctactccc agaccgtgga aaaattgtct tccacaaaac |
| 59521 | tggttcctgc tgccaaaaag gttgggacca ctgccttaga gtttataatt tggggttagc |
| 59581 | acagcctata tttacctgag aatttcaatg ggttcactga tctttccaaa tgaaaaggct |
| 59641 | tcttacgaaa attatatcca aactgtcttt tctcttagtt taataaacct atcagtaagt |
| 59701 | ttttactgag tactgctatt acatttttct ctgttaagca ttatgggggc tcagacatga |
| 59761 | tccattccct caaagaactt acctttcagc tgaagactga ctagaatgag caaatacggt |
| 59821 | taacaattaa caagtgagta ggccagctcg gccaacatgg tgaaaccctg tctctactaa |
| 59881 | aaatacaaaa attagccggg catggtggtg ggcgcctgta atcccagcta cctcctgctg |
| 59941 | aggcaggaga attgcttgaa cccaggaggt ggagattgca gtgagccgag attgcaccat |
| 60001 | tgcactccag cctgggcgac agagcaagac tctgtcttgg gaaaaaaaca aaacaaaaca |
| 60061 | agtgagaagg gaatcaagta ctacgtaaga tgtaatgtgg aattttaggg aaggaaggca |
| 60121 | gtgtatgctg gagtaattag agaaaggggc atgcatgatt tgatgcttga actggatcat |
| 60181 | gaaggataag caagattttg gcagcaagtg agagggagag aggagtttgt cagaggaatg |
| 60241 | gaacaagtca ggaggcagta atgtgtacgg cactccaagg actctgtcct gatcggagca |
| 60301 | gaagtgatga agcattgagt agtttgagaa agttagctaa gaagggtgag ggcagatgtt |
| 60361 | ggagagagtt gagtatcaga gagaagacat tagatttgag cagatagaac aaaaatgcca |
| 60421 | ttgccagttt ttgtgtaaga gaatagtatt agggtggtta cccaggaagt ggtcatcaga |
| 60481 | gttgaatgga gcagaaagag tgtctgatac agcatcggga cgttgtggtc acaaaatgag |
| 60541 | gtggtgaggg ctgagccaag atggtggcag tgggatggat aaaaagggat ggccaggaca |
| 60601 | aatattttaa aggaaaaatt aacaggacat ctttactgac tggattggaa ggctatgcag |
| 60661 | gaaatatatt gtcaaacttg attccaggat ttctatccta tgcctgggtt gcccaaaata |
| 60721 | tcagggaacc attgttagaa aaggtaggag ataccactgt tccaacaaaa agtattgagt |
| 60781 | ttggtgttgc acccacttaa cttcaaggcc ttacaagtga gtagacagtt agttagaatt |
| 60841 | gcagaagtgc cactcagaga gcagggcttg caatgtgggg ttggactttg tcaccattgt |
| 60901 | gttaattcct aattctatgc agatgctcag cttgaggaat acccatgttt gggcttcaga |
| 60961 | atgaaagcca agtaatattt actcagatgc caattttccc tctgaaatat ttgctcatgg |
| 61021 | aactgagaga acaatatata aagcattaat tatttttctc ataaagttat taataaaaag |
| 61081 | ataagatcag tgaaaggcag agtaaactag aagccaagta tagaaaatgg tatcattcaa |
| 61141 | agactcatta ctgtagtggt gaaaacaaaa caattttcca acagcttaag atgcctcagt |
| 61201 | attttggacc atttttaagt agttagtgtg ggcacttagt aaatatgtat taaactatag |
| 61261 | ttcattaatt cttttttttt tttttttgag atggagtttc actctggtca cccaggctgc |
| 61321 | atttttgctt tcttagtgat atataaaatg tcgagtttca caatgatggt atcttagatt |
| 61381 | tgattaaata tggtattaaa aaatagctga tcacagaaag tctctaccag tgtgatgtag |
| 61441 | atggctaaag tattccacat ttgcaaactt ttattgacct aaataagagg tgccccttgg |
| 61501 | gttgttttta tttggactgg gaatattagg agaaagcttt ttcattcagt gtgtaagtac |
| 61561 | aatctaccag aaatagaaac ccccatggac gatctatttc tttgatggta caggactcag |
| 61621 | aacattcaca aagatttagt tgttagcgga atagacatct gtattttatt caaaccaatt |
| 61681 | ttcccttcct aatctgagaa cattgtgcaa tctaagcagt tctaagcatg tttgctattc |
| 61741 | gtgcaaagtg agagtaaatc taaaagaaat ttttttgtgt gtttagggat ggtaataaag |
| 61801 | tctcttagtg gttgaaaatg ttatttctta caaaagtgga gaacatttgc ttttcaatac |
| 61861 | cagagttttc agccatttct gcattctgac ctattgactg gaggtaggtt gcctttgaat |
| 61921 | tcagtaaaac ttcatgggca gaaacacagt tccttttcct acttatttgg atatcatgat |
| 61981 | ggccattgca tgtatgtgtc tttttgtaag tccatgcctc agaactgaga agtaggaata |
| 62041 | aaattagggt cagggctggg gatgctactc tttgctgctg agaaacacaa tgcttcaggt |
| 62101 | aagtgattct gaagtccttc accacctgac ggtaaccttg ggttggtcca taggtatgtt |
| 62161 | ttcattttgc ttgttcatcc attttaattg gcttcctaga gcatgcttgt agatgtagag |
| 62221 | ccaaatttag agtagagcaa ccctctggca aacaggaaga gattaatttt gtggtatgct |
| 62281 | tttaagggac ttcccaggaa acttcaaaag cagaaaaaga agcactagct gcctattcca |
| 62341 | aaatgtgtaa aacaccactc agctttttaa aagtaggata aactcagagc gcgcgcacac |
| 62401 | gcgcgcgcgc acacacacac acacacacag agagaacatc tctagtaaaa agaaaagttg |
| 62461 | agctttctta gctagatgtg tgtattagcc agaaaaagcc aaggagtgaa gggttttaga |
| 62521 | gaactggagg agataaagtg gagtctgcat atgggaggca tttgaaatgg acttaaatgt |
| 62581 | ctttttaatg ctgacttttt cagttttctc cttaccagac acattgtttt catgacatta |
| 62641 | gccccaggca tagacacatc attaaaatga acatgtcaaa aaatgatttc tgtttagaaa |
| 62701 | taagcaaaac attttcagtt gtgaccaccc aggtgtagaa taaagaacag tggaattggg |
| 62761 | agccctgagt tctaacataa actttcttca tgacataagg caagtcttct atggcctttg |
| 62821 | gtttccttac ctgtaaaaca ggatggctca atgaaattat ctttcttctt tgctataata |
| 62881 | gagtatctct gtgggaagag gaaaaaaaaa gtcaatttaa aggctcctta tagttcccca |
| 62941 | actgctgttt tattgtgcta ttcatgccta gacatcacat agctagaaag gcccatcaga |
| 63001 | cccctcaggc cactgctgtt cctgtcacac attcctgcaa aggaccatgt tgctaacttg |
| 63061 | aaaaaaatta ctattaatta cacttgcagt tgttgcttag taacatttat gattttgtgt |
| 63121 | ttctcgtgac agcatgagca gagatcatta aaaattaaac ttacaaagct gctaaagtgg |
| 63181 | gaagaaggag aacttgaagc cacaattttt gcacttgctt agaagccatc taatctcagg |
| 63241 | tttatatgct agatcttggg ggaaacactg catgtctctg gtttatatta aaccacatac |
| 63301 | agcacactac tgacactgat ttgtgtctgg tgcagctgga gtttatcacc aagacataaa |
| 63361 | aaaaccttga ccctgcagaa tggcctggaa ttacaatcag atgggccaca tggcatcccg |
| 63421 | gtgaaagaaa gccctaacca gttttctgtc ttgtttctgc tttctcccta cagttccacc |
| 63481 | aggtgagaag agtgatgacc atccttttcc ttactatggt tatttcatac tttggttgca |
| 63541 | tgaaggctgc ccccatgaaa gaagcaaaca tccgaggaca aggtggcttg gcctacccag |
| 63601 | gtgtgcggac ccatgggact ctggagagcg tgaatgggcc caaggcaggt tcaagaggct |
| 63661 | tgacatcatt ggctgacact ttcgaacacg tgatagaaga gctgttggat gaggaccaga |
| 63721 | aagttcggcc caatgaagaa aacaataagg acgcagactt gtacacgtcc agggtgatgc |
| 63781 | tcagtagtca agtgcctttg gagcctcctc ttctctttct gctggaggaa tacaaaaatt |
| 63841 | acctagatgc tgcaaacatg tccatgaggg tccggcgcca ctctgaccct gcccgccgag |
| 63901 | gggagetgag cgtgtgtgac agtattagtg agtgggtaac ggcggcagac aaaaagactg |
| 63961 | cagtggacat gtcgggcggg acggtcacag tccttgaaaa ggtccctgta tcaaaaggcc |
| 64021 | aactgaagca atacttctac gagaccaagt gcaatcccat gggttacaca aaagaaggct |
| 64081 | gcaggggcat agacaaaagg cattggaact cccagtgccg aactacccag tcgtacgtgc |
| 64141 | gggcccttac catggatagc aaaaagagaa ttggctggcg attcataagg atagacactt |
| 64201 | cttgtgtatg tacattgacc attaaaaggg gaagatagtg gatttatgtt gtatagatta |
| 64261 | gattatattg agacaaaaat tatctatttg tatatataca taacagggta aattattcag |
| 64321 | ttaagaaaaa aataatttta tgaactgcat gtataaatga agtttataca gtacagtggt |
| 64381 | tctacaatct atttattgga catgtccatg accagaaggg aaacagtcat ttgcgcacaa |
| 64441 | cttaaaaagt ctgcattaca ttccttgata atgttgtggt ttgttgccgt tgccaagaac |
| 64501 | tgaaaacata aaaagttaaa aaaaataata aattgcatgc tgctttaatt gtgaattgat |
| 64561 | aataaactgt cctctttcag aaaacagaaa aaaacacaca cacacacaac aaaaatttga |
| 64621 | accaaaacat tccgtttaca ttttagacag taagtatctt cgttcttgtt agtactatat |
| 64681 | ctgttttact gcttttaact tctgatagcg ttggaattaa aacaatgtca aggtgctgtt |
| 64741 | gtcattgctt tactggctta ggggatgggg gatggggggt atatttttgt ttgttttgtg |
| 64801 | tttttttttc gtttgtttgt tttgtttttt agttcccaca gggagtagag atggggaaag |
| 64861 | aattcctaca atatatattc tggctgataa aagatacatt tgtatgttgt gaagatgttt |
| 64921 | gcaatatcga tcagatgact agaaagtgaa taaaaattaa ggcaactgaa caaaaaaatg |
| 64981 | ctcacactcc acatcccgtg atgcacctcc caggccccgc tcattctttg ggcgttggtc |
| 65041 | agagtaagct gcttttgacg gaaggaccta tgtttgctca gaacacattc tttccccccc |
| 65101 | tccccctctg gtctcctctt tgttttgttt taaggaagaa aaatcagttg cgcgttctga |
| 65161 | aatattttac cactgctgtg aacaagtgaa cacattgtgt cacatcatga cactcgtata |
| 65221 | agcatggaga acagtgattt ttttttagaa cagaaaacaa caaaaaataa ccccaaaatg |
| 65281 | aagattattt tttatgagga gtgaacattt gggtaaatca tggctaagct taaaaaaaac |
| 65341 | tcatggtgag gcttaacaat gtcttgtaag caaaaggtag agccctgtat caacccagaa |
| 65401 | acacctagat cagaacagga atccacattg ccagtgacat gagactgaac agccaaatgg |
| 65461 | aggctatgtg gagttggcat tgcatttacc ggcagtgcgg gaggaatttc tgagtggcca |
| 65521 | tcccaaggtc taggtggagg tggggcatgg tatttgagac attccaaaac gaaggcctct |
| 65581 | gaaggaccct tcagaggtgg ctctggaatg acatgtgtca agctgcttgg acctcgtgct |
| 65641 | ttaagtgcct acattatcta actgtgctca agaggttctc gactggagga ccacactcaa |
| 65701 | gccgacttat gcccaccatc ccacctctgg ataattttgc ataaaattgg attagcctgg |
| 65761 | agcaggttgg gagccaaatg tggcatttgt gatcatgaga ttgatgcaat gagatagaag |
| 65821 | atgtttgcta cctgaacact tattgctttg aaactagact tgaggaaacc agggtttatc |
| 65881 | ttttgagaac ttttggtaag ggaaaaggga acaggaaaag aaaccccaaa ctcaggccga |
| 65941 | atgatcaagg ggacccatag gaaatcttgt ccagagacaa gacttcggga aggtgtctgg |
| 66001 | acattcagaa caccaagact tgaaggtgcc ttgctcaatg gaagaggcca ggacagagct |
| 66061 | gacaaaattt tgctccccag tgaaggccac agcaaccttc tgcccatcct gtctgttcat |
| 66121 | ggagagggtc cctgcctcac ctctgccatt ttgggttagg agaagtcaag ttgggagcct |
| 66181 | gaaatagtgg ttcttggaaa aatggatccc cagtgaaaac tagagctcta agcccattca |
| 66241 | gcccatttca cacctgaaaa tgttagtgat caccacttgg accagcatcc ttaagtatca |
| 66301 | gaaagcccca agcaattgct gcatcttagt agggtgaggg ataagcaaaa gaggatgttc |
| 66361 | accataaccc aggaatgaag ataccatcag caaagaattt caatttgttc agtctttcat |
| 66421 | ttagagctag tctttcacag taccatctga atacctcttt gaaagaagga agactttacg |
| 66481 | tagtgtagat ttgttttgtg ttgtttgaaa atattatctt tgtaattatt tttaatatgt |
| 66541 | aaggaatgct tggaatatct gctatatgtc aactttatgc agcttccttt tgagggacaa |
| 66601 | atttaaaaca aacaaccccc catcacaaac ttaaaggatt gcaagggcca gatctgttaa |
| 66661 | gtggtttcat aggagacaca tccagcaatt gtgtggtcag tggctctttt acccaataag |
| 66721 | atacatcaca gtcacatgct tgatggttta tgttgaccta agatttattt tgttaaaatc |
| 66781 | tctctctgtt gtgttcgttc ttgttctgtt ttgttttgtt ttttaaagtc ttgctgtggt |
| 66841 | ctctttgtgg cagaagtgtt tcatgcatgg cagcaggcct gttgcttttt tatggcgatt |
| 66901 | cccattgaaa atgtaagtaa atgtctgtgg ccttgttctc tctatggtaa agatattatt |
| 66961 | caccatgtaa aacaaaaaac aatatttatt gtattttagt atatttatat aattatgtta |
| 67021 | ttgaaaaaaa ttggcattaa aacttaaccg catcagaacc tattgtaaat acaagttcta |
| 67081 | tttaagtgta ctaattaaca tataatatat gttttaaata tagaattttt aatgttttta |
| 67141 | aatatatttt caaagtacat aaaa |

Hair Cell Differentiation-Suppressing Gene

The term “hair cell differentiation-suppressing gene” refers to a gene encoding a protein (e.g., a transcription factor) that positively contributes (directly or indirectly) to the suppression of hair cell differentiation from supporting cells in a primate (e.g., a human). Non-limiting examples of hair cell differentiation-suppressing genes include: HES1, HES5, CDKN1B, and SOX2.

The term “mutation in a hair cell differentiation-suppressing gene” refers to a modification in a hair cell differentiation-suppressing gene that results in the production of a hair cell differentiation-suppressing protein having one or more of: one or more amino acid substitutions, and one or more amino acid insertions as compared to the wildtype hair cell differentiation-suppressing protein, and/or results in an increase in the expressed level of the encoded hair cell differentiation-suppressing protein in a primate cell as compared to the expressed level of the encoded hair cell differentiation-suppressing protein in a primate cell not having a mutation. In some embodiments, the mutation can result in the gain (or an increase in the level) of expression of a hair cell differentiation-suppressing mRNA or a hair cell differentiation-suppressing protein, or both the mRNA and protein. In some embodiments, the mutation can result in the production of an altered hair cell differentiation-suppressing protein having a gain or increase in one or more biological activities (functions) as compared to a wildtype hair cell differentiation-suppressing protein.

In some embodiments, the mutation is an insertion of one or more nucleotides into a hair cell differentiation-suppressing gene. In some embodiments, the mutation is in a regulatory sequence of the hair cell differentiation-suppressing gene, i.e., a portion of the gene that is not coding sequence. In some embodiments, a mutation in a regulatory sequence may be in a promoter or enhancer region and prevent or reduce the proper transcription of the hair cell differentiation-suppressing gene (e.g., a mutation in a regulatory sequence that increases the transcription of the hair cell differentiation-suppressing gene).

Hes Family Basic Helix-Loop-Helix (bHLH) Transcription Factor 1 (HES1)

The HES1 gene encodes hes family bHLH transcription factor 1, and acts as a transcriptional repressor. HES1 binds to the ATOH1 promoter to inhibit transcription in supporting cells and drives lateral inhibition (Abdolazimi et al. (2016) Development 143:841-850). Loss of HES1 results in supernumerary inner hair cells in early development. HES1 inhibition after damage induces hair cell regeneration (Du et al. (2018) Mol. Ther. 26(5):1313-1326).

The human HES1 gene is located on chromosome 3q29. It contains 4 exons encompassing ˜15 kilobases (kb) (NCBI Accession No. NM_005524). The full-length wildtype HES1 protein expressed from the human HES1 gene is 280 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype HES1 protein is or includes the sequence of SEQ ID NO: 36. Non-limiting examples of a nucleic acid encoding a wildtype HES1 protein is or includes SEQ ID NO: 37.

Human Full-length Wildtype HES1 Protein
(SEQ ID NO: 36)
MPADIMEKNSSSPVAATPASVNTTPDKPKTASEHRKSSKPIMEKRRRAR
INESLSQLKTLILDALKKDSSRHSKLEKADILEMTVKHLRNLQRAQMTA
ALSTDPSVLGKYRAGFSECMNEVTRFLSTCEGVNTEVRTRLLGHLANCM
TQINAMTYPGQPHPALQAPPPPPPGPGGPQHAPFAPPPPLVPIPGGAAP
PPGGAPCKLGSQAGEAAKVFGGFQVVPAPDGQFAFLIPNGAFAHSGPVI
PVYTSNSGTSVGPNAVSPSSGPSLTADSMWRPWRN
Human Wildtype HES1 cDNA
(SEQ ID NO: 37)
atgccagctgatataatggagaaaaattcctcgtccccggtggctgcta
ccccagccagtgtcaacacgacaccggataaaccaaagacagcatctga
gcacagaaagtcatcaaagcctattatggagaaaagacgaagagcaaga
ataaatgaaagtctgagccagctgaaaacactgattttggatgctctga
agaaagatagctcgcggcattccaagctggagaaggcggacattctgga
aatgacagtgaagcacctccggaacctgcagcgggcgcagatgacggct
gcgctgagcacagacccaagtgtgctggggaagtaccgagccggcttca
gcgagtgcatgaacgaggtgacccgcttcctgtccacgtgcgagggcgt
taataccgaggtgcgcactcggctgctcggccacctggccaactgcatg
acccagatcaatgccatgacctaccccgggcagccgcaccccgccttgc
aggcgccgccaccgcccccaccgggacccggcggcccccagcacgcgcc
gttcgcgccgccgccgccactcgtgcccatccccgggggcgcggcgccc
cctcccggcggcgccccctgcaagctgggcagccaggctggagaggcgg
ctaaggtgtttggaggcttccaggtggtaccggctcccgatggccagtt
tgctttcctcattcccaacggggccttcgcgcacagcggccctgtcatc
cccgtctacaccagcaacagcggcacctccgtgggccccaacgcagtgt
caccttccagcggcccctcgcttacggcggactccatgtggaggccgtg
gcggaactga

A non-limiting example of a human wildtype HES1 genomic DNA sequence is SEQ ID NO: 38. The exons in SEQ ID NO: 38 are: nucleotide positions 1-347 (exon 1), nucleotide positions 348-443 (exon 2), nucleotide positions 444-531 (exon 3), and nucleotide positions 532-1461 (exon 4).

| Human Wildtype HES1 Gene | |
|---|---|
| (SEQ ID NO: 38) | |
| 1 | gggatcacac aggatccgga gctggtgctg ataacagcgg aatcccccgt ctacctctct |
| 61 | ccttggtcct ggaacagcgc tactgatcac caagtagcca caaaatataa taaaccctca |
| 121 | gcacttgctc agtagttttg tgaaagtctc aagtaaaaga gacacaaaca aaaaattctt |
| 181 | tttcgtgaag aactccaaaa ataaaattct ctagagataa aaaaaaaaaa aaaaggaaaa |
| 241 | tgccagctga tataatggag aaaaattcct cgtccccggt ggctgctacc ccagccagtg |
| 301 | tcaacacgac accggataaa ccaaagacag catctgagca cagaaagtca tcaaagccta |
| 361 | ttatggagaa aagacgaaga gcaagaataa atgaaagtct gagccagctg aaaacactga |
| 421 | ttttggatgc tctgaagaaa gatagctcgc ggcattccaa gctggagaag gcggacattc |
| 481 | tggaaatgac agtgaagcac ctccggaacc tgcagcgggc gcagatgacg gctgcgctga |
| 541 | gcacagaccc aagtgtgctg gggaagtacc gagccggctt cagcgagtgc atgaacgagg |
| 601 | tgacccgctt cctgtccacg tgcgagggcg ttaataccga ggtgcgcact cggctgctcg |
| 661 | gccacctggc caactgcatg acccagatca atgccatgac ctaccccggg cagccgcacc |
| 721 | ccgccttgca ggcgccgcca ccgcccccac cgggacccgg cggcccccag cacgcgccgt |
| 781 | tcgcgccgcc gccgccactc gtgcccatcc ccgggggcgc ggcgccccct cccggcggcg |
| 841 | ccccctgcaa gctgggcagc caggctggag aggcggctaa ggtgtttgga ggcttccagg |
| 901 | tggtaccggc tcccgatggc cagtttgctt tcctcattcc caacggggcc ttcgcgcaca |
| 961 | gcggccctgt catccccgtc tacaccagca acagcggcac ctccgtgggc cccaacgcag |
| 1021 | tgtcaccttc cagcggcccc tcgcttacgg cggactccat gtggaggccg tggcggaact |
| 1081 | gagggggctc aggccacccc tcctcctaaa ctccccaacc cacctctctt ccctccggac |
| 1141 | tctaaacagg aacttgaata ctgggagaga agaggacttt tttgattaag tggttacttt |
| 1201 | gtgttttttt aatttctaag aagttacttt ttgtagagag agctgtatta agtgactgac |
| 1261 | catgcactat atttgtatat attttatatg ttcatattgg attgcgcctt tgtattataa |
| 1321 | aagctcagat gacatttcgt tttttacacg agatttcttt tttatgtgat gccaaagatg |
| 1381 | tttgaaaatg ctcttaaaat atcttccttt ggggaagttt atttgagaaa atataataaa |
| 1441 | agaaaaaagt aaaggctttt aaaaaaaaaa aaaaa |

Non-limiting examples of siRNA targeting HES1 are described in, e.g., Zhang et al., World J. Gastroenterol. 24(29):3260-3272, 2018; Du et al., Mol. Ther. 26(5):1313-1326, 2018; Li et al., Oncol. Lett. 14(4):3989-3996, 2017; and Du et al., Hear Res. 304:91-110, 2013. Non-limiting examples of shRNA targeting HES1 are described in, e.g., Cenciarelli et al., Oncotarget 8(11):17873-17886, 2017, and Wang et al., Oncotarget 6(34):36713-36730, 2015.

Hes Family bHLH Transcription Factor 5 (HES5)

The HES5 gene encodes hes family bHLH transcription 5, and acts as a transcriptional repressor. HES5 is a Notch-pathway activator, and binds the ATOH1 promoter to inhibit transcription in supporting cells. Loss of HES5 results in supernumerary outer hair cells in early development. HES5 inhibition in adult mouse utricle results in increased regeneration after aminoglycoside damage (Jung et al. (2013) Mol. Ther. 21(4):834-841; Abdolazimi et al. (2016) Development 143:841-850).

The human HES5 gene is located on chromosome 1p36. It contains 3 exons encompassing ˜18 kilobases (kb) (NCBI Accession No. NM_001010926.3). The full-length wildtype HES5 protein expressed from the human HES5 gene is 166 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype HES5 protein is or includes the sequence of SEQ ID NO: 39. Non-limiting examples of a nucleic acid encoding a wildtype HES5 protein is or includes SEQ ID NO: 40.

Human Full-length Wildtype HESS Protein
(SEQ ID NO: 39)
MAPSTVAVELLSPKEKNRLRKPVVEKMRRDRINSSIEQLKLLLEQEFARHQPNSKLEKADILEMAVSYLKHSKAFVA
AAGPKSLHQDYSEGYSWCLQEAVQFLTLHAASDTQMKLLYHFQRPPAAPAAPAKEPKAPGAAPPPALSAKATAAAAA
AHQPACGLWRPW
Human Wildtype HES5 cDNA
(SEQ ID NO: 40)
atggcccccagcactgtggccgtggagctgctcagccccaaagagaaaaaccgactgcggaagccggtggtggagaa
gatgcgccgcgaccgcatcaacagcagcatcgagcagctgaagctgctgctggagcaggagttcgcgcggcaccagc
ccaactccaagctggagaaggccgacatcctggagatggctgtcagctacctgaagcacagcaaagccttcgtcgcc
gccgccggccccaagagcctgcaccaggactacagcgaaggctactcgtggtgcctgcaggaggccgtgcagttcct
gacgctccacgccgccagcgacacgcagatgaagctgctgtaccacttccagcggcccccggccgcgcccgccgcgc
ccgccaaggagcccaaggcgccgggcgccgcgcccccgcccgcgctctccgccaaggccaccgccgccgccgccgcc
gcgcaccagcccgcctgcggcctctggcggccctggtga

A non-limiting example of a human wildtype HES5 genomic DNA sequence is SEQ ID NO: 41. The exons in SEQ ID NO: 41 are: nucleotide positions 1-135 (exon 1), nucleotide positions 136-301 (exon 2), and nucleotide positions 302-1306 (exon 3).

| Human Wildtype HES5 Gene | |
|---|---|
| (SEQ ID NO: 41) | |
| 1 | cgcgcttggc cttgcccgcg cccgctcgcc tcgtctcgcc cggcctcccc gcgtcgcctc |
| 61 | ctcgcctgtt ccgcgccagg catggccccc agcactgtgg ccgtggagct gctcagcccc |
| 121 | aaagagaaaa accgactgcg gaagccggtg gtggagaaga tgcgccgcga ccgcatcaac |
| 181 | agcagcatcg agcagctgaa gctgctgctg gagcaggagt tcgcgcggca ccagcccaac |
| 241 | tccaagctgg agaaggccga catcctggag atggctgtca gctacctgaa gcacagcaaa |
| 301 | gccttcgtcg ccgccgccgg ccccaagagc ctgcaccagg actacagcga aggctactcg |
| 361 | tggtgcctgc aggaggccgt gcagttcctg acgctccacg ccgccagcga cacgcagatg |
| 421 | aagctgctgt accacttcca gcggcccccg gccgcgcccg ccgcgcccgc caaggagccc |
| 481 | aaggcgccgg gcgccgcgcc cccgcccgcg ctctccgcca aggccaccgc cgccgccgcc |
| 541 | gccgcgcacc agcccgcctg cggcctctgg cggccctggt gacccggcgg gacctgcggg |
| 601 | cgcgcggccc gacgaccaga gggcgagcct gctcctctcg cctgtaggga agcgccttcc |
| 661 | cgccgtcgtc cgccccgggc ttggacgcgc ccttctccgg aaggctctgg ccccaagctg |
| 721 | gccggcccgc aggagcccca ttctcagaga atgtgtgtgc agagtccctg ccgttttagg |
| 781 | acaatcaggg cccatcttct gccaagtgtc tgaccccatg gggttgttct gtgtttgcat |
| 841 | ttaagcaagt gacttctggg aagtccccgg ccgcccgggg ttctatgata tttgtagtgc |
| 901 | cggggctcgc acactgctgc ccccagcctg tagaggactt tcttcagggc ccgtagctgc |
| 961 | tgggcgtacc cctggcaggc gggctgtgcc gcgggcacat ttgccttttg tgaaggccga |
| 1021 | actcgagctg tatcctcata ggaaacagtg atcaccccgg acgggcgtcc aggaccctga |
| 1081 | gggccatggc caaaaggctc ctgagtgtgc ctggtggtct ggctggggct cacggtgggc |
| 1141 | tgtctgggga gggtgggtgc ctccactatg atccttaaag gattcctctg tgtgggtgga |
| 1201 | tgcgtgtggg cacgactttg tactcagaaa ttgaactctc agtcacgtgg aagccacggg |
| 1261 | actgctccga agccgccata ataaaatctg attgttcagc ccccaaaaaa aaaaaaaaa |

Non-limiting examples of siRNA targeting HES5 are described in, e.g., Gu et al., Oncol. Rep. 37(1):474-482, 2017; Zhu et al., Exp. Mol. Pathol. 99(3):474-484, 2015; Du et al., Hear Res. 304:91-110, 2013; Jung et al., Mol. Ther. 21(4):834-841, 2013; and Liu et al., Int. J. Gynecol. Cancer 20(7):1109-1116, 2010. Non-limiting examples of shRNA targeting HES5 are described in, e.g., Lee et al., J. Neurochem. 100(6):1531-1542, 2007; and Osario et al., Development 140:1-12, 2013.

Cyclin Dependent Kinase Inhibitor 1B (Cdkn1b) (p27^{kip1})

The CDKN1B gene encodes a cyclin-dependent kinase inhibitor (p27^{kip1}). CDKN1B is a cell cycle regulator and controls the cell cycle exit of supporting cells. For example, p27^{kip1} binds to and prevents activation of cyclin E (CDK2) and cyclin D (CDK4). Inhibition of CDKN1B promotes supporting cell proliferation and regeneration induction through its canonical pathway and a non-canonical pathway that involves Gata3 (Minoda et al. (2007) Hear Res. 232(1-2):44-51; Walters et al. (2014) J. Neurosci 34(47):15751-15763; Walters et al. (2017) Cell Rep 19(2):307-320).

The human CDKN1B gene is located on chromosome 12p13. It contains 3 exons encompassing ˜5 kilobases (kb) (NCBI Accession No. NG_016341.1). The full-length wildtype CDKN1B protein expressed from the human CDKN1B gene is 198 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype CDKN1B (p27^{kip1}) protein is or includes the sequence of SEQ ID NO: 42. Non-limiting examples of a nucleic acid encoding a wildtype CDKN1B (p27) protein is or includes SEQ ID NO: 43.

Human Full-length Wildtype CDKN1B (p27^{kip1}) Protein
(SEQ ID NO: 42)
MSNVRVSNGSPSLERMDARQAEHPKPSACRNLFGPVDHEELTRDLEKHCRDMEEASQRKWNFDFQNHKPLEGKYEWQ
EVEKGSLPEFYYRPPRPPKGACKVPAQESQDVSGSRPAAPLIGAPANSEDTHLVDPKTDPSDSQTGLAEQCAGIRKR
PATDDSSTQNKRANRTEENVSDGSPNAGSVEQTPKKPGLRRRQT
Human Wildtype CDKN1B (p27^{kip1}) cDNA
(SEQ ID NO: 43)
atgtcaaacgtgcgagtgtctaacgggagccctagcctggagcggatggacgccaggcaggcggagcaccccaagcc
ctcggcctgcaggaacctcttcggcccggtggaccacgaagagttaacccgggacttggagaagcactgcagagaca
tggaagaggcgagccagcgcaagtggaatttcgattttcagaatcacaaacccctagagggcaagtacgagtggcaa
gaggtggagaagggcagcttgcccgagttctactacagacccccgcggccccccaaaggtgcctgcaaggtgccggc
gcaggagagccaggatgtcagcgggagccgcccggcggcgcctttaattggggctccggctaactctgaggacacgc
atttggtggacccaaagactgatccgtcggacagccagacggggttagcggagcaatgcgcaggaataaggaagcga
cctgcaaccgacgattcttctactcaaaacaaaagagccaacagaacagaagaaaatgtttcagacggttccccaaa
tgccggttctgtggagcagacgcccaagaagcctggcctcagaagacgtcaaacgtaa

A non-limiting example of a human wildtype CDKN1B (p27^{kip1}) genomic DNA sequence is SEQ ID NO: 44. The exons in SEQ ID NO: 44 are: nucleotide positions 1-1045 (exon 1), nucleotide positions 1556-1685 (exon 2), and nucleotide positions 3767-5114 (exon 3). The introns in SEQ ID NO: 44 are: nucleotide positions 1046-1555 (intron 1) and nucleotide positions 1686-3766 (intron 2).

| Human Wildtype CDKN1B (p27^{kip1}) Gene | |
|---|---|
| (SEQ ID NO: 44) | |
| 1 ttaaggccgc gctcgccagc ctcggcgggg cggctcccgc cgccgcaacc aatggatctc | 61 |
| ctcctctgtt taaatagact cgccgtgtca atcattttct tcttcgtcag cctcccttcc | 121 |
| accgccatat tgggccacta aaaaaagggg gctcgtcttt tcggggtgtt tttctccccc | 181 |
| tcccctgtcc ccgcttgctc acggctctgc gactccgacg ccggcaaggt ttggagagcg | 241 |
| gctgggttcg cgggacccgc gggcttgcac ccgcccagac tcggacgggc tttgccaccc | 301 |
| tctccgcttg cctggtcccc tctcctctcc gccctcccgc tcgccagtcc atttgatcag | 361 |
| cggagactcg gcggccgggc cggggcttcc ccgcagcccc tgcgcgctcc tagagctcgg | 421 |
| gccgtggctc gtcggggtct gtgtcttttg gctccgaggg cagtcgctgg gcttccgaga | 481 |
| ggggttcggg ctgcgtaggg gcgctttgtt ttgttcggtt ttgttttttt gagagtgcga | 541 |
| gagaggcggt cgtgcagacc cgggagaaag atgtcaaacg tgcgagtgtc taacgggagc | 601 |
| cctagcctgg agcggatgga cgccaggcag gcggagcacc ccaagccctc ggcctgcagg | 661 |
| aacctcttcg gcccggtgga ccacgaagag ttaacccggg acttggagaa gcactgcaga | 721 |
| gacatggaag aggcgagcca gcgcaagtgg aatttcgatt ttcagaatca caaaccccta | 781 |
| gagggcaagt acgagtggca agaggtggag aagggcagct tgcccgagtt ctactacaga | 841 |
| cccccgcggc cccccaaagg tgcctgcaag gtgccggcgc aggagagcca ggatgtcagc | 901 |
| gggagccgcc cggcggcgcc tttaattggg gctccggcta actctgagga cacgcatttg | 961 |
| gtggacccaa agactgatcc gtcggacagc cagacggggt tagcggagca atgcgcagga | 1021 |
| ataaggaagc gacctgcaac cgacggtaat gaccctttcc caaccataga atgtgtttgg | 1081 |
| ggccccgctt tgcctgctgg agggtgttaa ccttagcttg cttttcggcg tattctgatt | 1141 |
| tagctttggg agagctaact ttattggtct taggtgttca gtgctacctg gcccactgct | 1201 |
| tgtctgtttg tgacttttaa gtcagaaact ggagatggta agatccgata atttccctaa | 1261 |
| cttaatacat cgcggtccct ctcactagca actcctaggt atgtgacaaa gttgggatgt | 1321 |
| ttatcaacgg tccgcctcct ggctagggaa agagctctgg ggcggagaat gcactttctg | 1381 |
| ttttttgaaa acaacctcat tttgtgccct taaaagccac tggggatgac ggatccagga | 1441 |
| ttgtgggtgg aggtagtggg tttttcatcc cctgactatg gggccaactt ctgccagcca | 1501 |
| ttgttttttc taataaagat tgtgtgttct ttttaaaaat ttcccctgcg cttagattct | 1561 |
| tctactcaaa acaaaagagc caacagaaca gaagaaaatg tttcagacgg ttccccaaat | 1621 |
| gccggttctg tggagcagac gcccaagaag cctggcctca gaagacgtca aacgtaaaca | 1681 |
| gctcggtggg ttgatcacta aaggagcacg cactggaacc cggggccttc agacctcacg | 1741 |
| atacctgatc ttactggttg ctggcaaatt aaaagcttat ggggttttgt tttgtttata | 1801 |
| cttcgtgagg tcaaaaaagt agcaatgggg aaggctgggg atacggtaat tcctcagagt | 1861 |
| ttctatgccc agagatactt tctcttcaaa ctgttgacca gagcagctac ttgtaaccca | 1921 |
| ggccccatcg ggtaggaagg tcgtttccct gtgagtccca ctaaaacgtg ttgggagcaa | 1981 |
| taggttcttt gcccatccga acaagaacta gggtactccc tcagtccgaa ttaatgagaa | 2041 |
| ttaatttcct agaggttcag cttgagtcgg taacagattt tgagccatac atggaaaaat | 2101 |
| ggcaaataca tgattaagtt tcaattttga gggggaatgt ttggtagaaa ttgctcatct | 2161 |
| ttggttatgc aagggattag agatgtgaat aggatggtat gttgtgttct ttgacatttt | 2221 |
| aataaactgt cactttccct gttgtctcct aagtttggag agagaaggaa ccagtatttg | 2281 |
| caaaaaccaa atggaaagat aaaaaagtta ctaaagtttc tacagaattt ctggtaacac | 2341 |
| tgaagttgca aagcagaagt taaattaact cttgtcagta agcaatccag gaacacgtca | 2401 |
| gccagtgtat gctaattgtg ccgtaacagg gtgatttgga tatttgtagg ggaaatgggt | 2461 |
| agtaaatatc aagactggtg accgtaggtc agcccagcac aaaggaagtg gagatttttc | 2521 |
| catgcacaag aatctgatca ctgtaaatag ctaatttgaa taattcagtc cccagataac | 2581 |
| caacatgggt tggttattca taataaacta catattttaa tagtttatta gcttccttta | 2641 |
| gaccaagact gtgacctctt tattttctaa agcacacacg tagtttagca tatgaggcga | 2701 |
| taaaatattg atgttaactt tttaaatccc cagttataaa aattttaaaa taacagggat | 2761 |
| taaggtgaga ttcaggtttg ttgtgtcttt aaattgtata tgtgacttca catatctttt | 2821 |
| tcagcgctta tacaaaacgg cactatagaa cctccatttt acagcaccat atgaagtggg | 2881 |
| aaaattaggt gaaaattttc ctgaagcaac cttaacatgc gcagcccttg ttggtttgtg | 2941 |
| acttgtggcc tagctcatca gatgagccac gagaatcaga cctggatttt gatctggccc | 3001 |
| tgttctgaca tgcaatgagg catttgtagg atttagtaat attgctagtt caaagaatac | 3061 |
| tagaaatatt agtaagaacc tattcaaaag tattcatgag tattttctgc atatgaatca | 3121 |
| ggaattagaa tattttgaaa atgatgttaa taaaattttc ctctggaagg cctttataat | 3181 |
| ttttattccc aatcattttt caaatttaga aagtttaatc tgtcacagga gaaaaaaaat | 3241 |
| taaaaatttt caaaaattta gaaaattttt acccgtaagt attacagttt cctaattatc | 3301 |
| ctatttattt cccacttgcc tttgacctag attatttaga gtagggtttc tcagcctctg | 3361 |
| cactaatgac attttgggcc gaataattct gttgtaggag gctgtcctgt gtgttttaga | 3421 |
| ttgtttggaa ttatccctgg cctctcacac tagatgccag cagtatcctc ctcccccagt | 3481 |
| gtgacaacct aaaatgtctc cagacattgc caaatgttcc ctgtggggcg ggggcaacat | 3541 |
| tgcctactgt taagaactac tgctctagac caaagaacac agcacagagg aaaggaaaaa | 3601 |
| aaaatccagt taagagaatg ttaggtggag atgactatag tcatcaaact tttttcccca | 3661 |
| tcaagtattt ccaagctaac atagtgacaa aataattcct gtactctact ggtaacgtta | 3721 |
| atctagtgct cttcctttaa ttcttccgtt ttgttttctt ttgcagaatt aagaatatgt | 3781 |
| ttccttgttt atcagataca tcactgcttg atgaagcaag gaagatatac atgaaaattt | 3841 |
| taaaaataca tatcgctgac ttcatggaat ggacatcctg tataagcact gaaaaacaac | 3901 |
| aacacaataa cactaaaatt ttaggcactc ttaaatgatc tgcctctaaa agcgttggat | 3961 |
| gtagcattat gcaattaggt ttttccttat ttgcttcatt gtactacctg tgtatatagt | 4021 |
| ttttaccttt tatgtagcac ataaactttg gggaagggag ggcagggtgg ggctgaggaa | 4081 |
| ctgacgtgga gcggggtatg aagagcttgc tttgatttac agcaagtaga taaatatttg | 4141 |
| acttgcatga agagaagcaa ttttggggaa gggtttgaat tgttttcttt aaagatgtaa | 4201 |
| tgtccctttc agagacagct gatacttcat ttaaaaaaat cacaaaaatt tgaacactgg | 4261 |
| ctaaagataa ttgctattta tttttacaag aagtttattc tcatttggga gatctggtga | 4321 |
| tctcccaagc tatctaaagt ttgttagata gctgcatgtg gcttttttaa aaaagcaaca | 4381 |
| gaaacctatc ctcactgccc tccccagtct ctcttaaagt tggaatttac cagttaatta | 4441 |
| ctcagcagaa tggtgatcac tccaggtagt ttggggcaaa aatccgaggt gcttgggagt | 4501 |
| tttgaatgtt aagaattgac catctgcttt tattaaattt gttgacaaaa ttttctcatt | 4561 |
| ttcttttcac ttcgggctgt gtaaacacag tcaaaataat tctaaatccc tcgatatttt | 4621 |
| taaagatctg taagtaactt cacattaaaa aatgaaatat tttttaattt aaagcttact | 4681 |
| ctgtccattt atccacagga aagtgttatt tttcaaggaa ggttcatgta gagaaaagca | 4741 |
| cacttgtagg ataagtgaaa tggatactac atctttaaac agtatttcat tgcctgtgta | 4801 |
| tggaaaaacc atttgaagtg tacctgtgta cataactctg taaaaacact gaaaaattat | 4861 |
| actaacttat ttatgttaaa agattttttt taatctagac aatatacaag ccaaagtggc | 4921 |
| atgttttgtg catttgtaaa tgctgtgttg ggtagaatag gttttcccct cttttgttaa | 4981 |
| ataatatggc tatgcttaaa aggttgcata ctgagccaag tataattttt tgtaatgtgt | 5041 |
| gaaaaagatg ccaattattg ttacacatta agtaatcaat aaagaaaact tccatagcta | 5101 |
| ttcattgagt caaa | |

Non-limiting examples of siRNA targeting CDKN1B (p27^{kip1}) are described in, e.g., Galardi et al., J. Biol. Chem. 282:23716-23724, 2007; Liang et al., Nature Cell Biol. 9:218-224, 2007; Tamamori-Adachi et al., J. Biol. Chem. 279:50429-50436, 2004; Akashiba et al., Cell. Mol. Life Sci. 63:2397-2404, 2006; and Lee et al., J. Mol. Med. 83(4):296-307, 2005. Non-limiting examples of shRNA targeting CDKN1B (p27^{kip1}) are described in, e.g., Lin et al., Nature 464:374-379, 2010.

Sex Determining Region Y—Box 2 (SOX2)

The SOX2 gene encodes the sex determining region Y— box 2 protein. SOX2 is a transcription factor that binds the ATOH1 3′-enhancer and activates initial hair cell differentiation. Low SOX2 expression levels are required for proper hair cell maturation. Haploinsufficiency of SOX2 results in a few extra inner hair cells. SOX2 also increases the susceptibility to induce transdifferentiation in the presence of other contributing components, e.g., beta-catenin (Kempfle et al. (2016) Sci Rep 6:23293; Atkinson et al. (2018) J Clin Invest 128(4):1641-1656).

The human SOX2 gene is located on chromosome 3q26. It contains 1 exon encompassing ˜3 kilobases (kb) (NCBI Accession No. NG_009080.1). The full-length wildtype SOX2 protein expressed from the human SOX2 gene is 317 amino acids in length.

Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype SOX2 protein is or includes the sequence of SEQ ID NO: 45. Non-limiting examples of a nucleic acid encoding a wildtype SOX2 protein is or includes SEQ ID NO: 46. As can be appreciated in the art, at least some or all of the codons in SEQ ID NO: 46 can be codon-optimized to allow for optimal expression in a non-human primate.

Human Full-length Wildtype SOX2 Protein
(SEQ ID NO: 45)
MYNMMETELKPPGPQQTSGGGGGNSTAAAAGGNQKNSPDRVKRPMNAFMV
WSRGQRRKMAQENPKMHNSEISKRLGAEWKLLSETEKRPFIDEAKRLRAL
HMKEHPDYKYRPRRKTKTLMKKDKYTLPGGLLAPGGNSMASGVGVGAGLG
AGVNQRMDSYAHMNGWSNGSYSMMQDQLGYPQHPGLNAHGAAQMQPMHRY
DVSALQYNSMTSSQTYMNGSPTYSMSYSQQGTPGMALGSMGSVVKSEASS
SPPVVTSSSHSRAPCQAGDLRDMISMYLPGAEVPEPAAPSRLHMSQHYQS
GPVPGTAINGTLPLSHM
Human Wildtype SOX2 cDNA
(SEQ ID NO: 46)
atgtacaacatgatggagacggagctgaagccgccgggcccgcagcaaac
ttcggggggcggcggcggcaactccaccgcggcggcggccggcggcaacc
agaaaaacagcccggaccgcgtcaagcggcccatgaatgccttcatggtg
tggtcccgcgggcagcggcgcaagatggcccaggagaaccccaagatgca
caactcggagatcagcaagcgcctgggcgccgagtggaaacttttgtcgg
agacggagaagcggccgttcatcgacgaggctaagcggctgcgagcgctg
cacatgaaggagcacccggattataaataccggccccggcggaaaaccaa
gacgctcatgaagaaggataagtacacgctgcccggcgggctgctggccc
ccggcggcaatagcatggcgagcggggtcggggtgggcgccggcctgggc
gcgggcgtgaaccagcgcatggacagttacgcgcacatgaacggctggag
caacggcagctacagcatgatgcaggaccagctgggctacccgcagcacc
cgggcctcaatgcgcacggcgcagcgcagatgcagcccatgcaccgctac
gacgtgagcgccctgcagtacaactccatgaccagctcgcagacctacat
gaacggctcgcccacctacagcatgtcctactcgcagcagggcacccctg
gcatggctcttggctccatgggttcggtggtcaagtccgaggccagctcc
agcccccctgtggttacctcttcctcccactccagggcgccctgccaggc
cggggacctccgggacatgatcagcatgtatctccccggcgccgaggtgc
cggaacccgccgcccccagcagacttcacatgtcccagcactaccagagc
ggcccggtgcccggcacggccattaacggcacactgcccctctcacacat
gtga

A non-limiting example of a human wildtype SOX2 genomic DNA sequence is SEQ ID NO: 47. The exon in SEQ ID NO: 47 is nucleotide positions 1-2520 (exon 1).

Human Wildtype SOX2 Gene
(SEQ ID NO: 47)

| | |
|---|---|
| 1 | ggatggttgt ctattaactt gttcaaaaaa gtatcaggag |
| | ttgtcaaggc agagaagaga |
| 61 | gtgtttgcaa aagggggaaa gtagtttgct gcctctttaa |
| | gactaggact gagagaaaga |
| 121 | agaggagaga gaaagaaagg gagagaagtt tgagccccag |
| | gcttaagcct ttccaaaaaa |
| 181 | taataataac aatcatcggc ggcggcagga tcggccagag |
| | gaggagggaa gcgctttttt |
| 241 | tgatcctgat tccagtttgc ctctctcttt ttttccccca |
| | aattattctt cgcctgattt |
| 301 | tcctcgcgga gccctgcgct cccgacaccc ccgcccgcct |
| | cccctcctcc tctccccccg |
| 361 | cccgcgggcc ccccaaagtc ccggccgggc cgagggtcgg |
| | cggccgccgg cgggccgggc |
| 421 | ccgcgcacag cgcccgcatg tacaacatga tggagacgga |
| | gctgaagccg ccgggcccgc |
| 481 | agcaaacttc ggggggcggc ggcggcaact ccaccgcggc |
| | ggcggccggc ggcaaccaga |
| 541 | aaaacagccc ggaccgcgtc aagcggccca tgaatgcctt |
| | catggtgtgg tcccgcgggc |
| 601 | agcggcgcaa gatggcccag gagaacccca agatgcacaa |
| | ctcggagatc agcaagcgcc |
| 661 | tgggcgccga gtggaaactt ttgtcggaga cggagaagcg |
| | gccgttcatc gacgaggcta |
| 721 | agcggctgcg agcgctgcac atgaaggagc acccggatta |
| | taaataccgg ccccggcgga |
| 781 | aaaccaagac gctcatgaag aaggataagt acacgctgcc |
| | cggcgggctg ctggcccccg |
| 841 | gcggcaatag catggcgagc ggggtcgggg tgggcgccgg |
| | cctgggcgcg ggcgtgaacc |
| 901 | agcgcatgga cagttacgcg cacatgaacg gctggagcaa |
| | cggcagctac agcatgatgc |
| 961 | aggaccagct gggctacccg cagcacccgg gcctcaatgc |
| | gcacggcgca gcgcagatgc |
| 1021 | agcccatgca ccgctacgac gtgagcgccc tgcagtacaa |
| | ctccatgacc agctcgcaga |
| 1081 | cctacatgaa cggctcgccc acctacagca tgtcctactc |
| | gcagcagggc acccctggca |
| 1141 | tggctcttgg ctccatgggt tcggtggtca agtccgaggc |
| | cagctccagc ccccctgtgg |
| 1201 | ttacctcttc ctcccactcc agggcgccct gccaggccgg |
| | ggacctccgg gacatgatca |
| 1261 | gcatgtatct ccccggcgcc gaggtgccgg aacccgccgc |
| | ccccagcaga cttcacatgt |
| 1321 | cccagcacta ccagagcggc ccggtgcccg gcacggccat |
| | taacggcaca ctgcccctct |
| 1381 | cacacatgtg agggccggac agcgaactgg aggggggaga |
| | aattttcaaa gaaaaacgag |
| 1441 | ggaaatggga ggggtgcaaa agaggagagt aagaaacagc |
| | atggagaaaa cccggtacgc |
| 1501 | tcaaaaagaa aaaggaaaaa aaaaaatccc atcacccaca |
| | gcaaatgaca gctgcaaaag |
| 1561 | agaacaccaa tcccatccac actcacgcaa aaaccgcgat |
| | gccgacaaga aaacttttat |
| 1621 | gagagagatc ctggacttct ttttggggga ctatttttgt |
| | acagagaaaa cctggggagg |
| 1681 | gtggggaggg cgggggaatg gaccttgtat agatctggag |
| | gaaagaaagc tacgaaaaac |
| 1741 | tttttaaaag ttctagtggt acggtaggag ctttgcagga |
| | agtttgcaaa agtctttacc |
| 1801 | aataatattt agagctagtc tccaagcgac gaaaaaaatg |
| | ttttaatatt tgcaagcaac |
| 1861 | ttttgtacag tatttatcga gataaacatg gcaatcaaaa |
| | tgtccattgt ttataagctg |
| 1921 | agaatttgcc aatatttttc aaggagaggc ttcttgctga |
| | attttgattc tgcagctgaa |
| 1981 | atttaggaca gttgcaaacg tgaaaagaag aaaattattc |
| | aaatttggac attttaattg |
| 2041 | tttaaaaatt gtacaaaagg aaaaaattag aataagtact |
| | ggcgaaccat ctctgtggtc |
| 2101 | ttgtttaaaa agggcaaaag ttttagactg tactaaattt |
| | tataacttac tgttaaaagc |
| 2161 | aaaaatggcc atgcaggttg acaccgttgg taatttataa |
| | tagcttttgt tcgatcccaa |
| 2221 | ctttccattt tgttcagata aaaaaaacca tgaaattact |
| | gtgtttgaaa tattttctta |
| 2281 | tggtttgtaa tatttctgta aatttattgt gatattttaa |
| | ggttttcccc cctttatttt |
| 2341 | ccgtagttgt attttaaaag attcggctct gtattatttg |
| | aatcagtctg ccgagaatcc |
| 2401 | atgtatatat ttgaactaat atcatcctta taacaggtac |
| | attttcaact taagttttta |
| 2461 | ctccattatg cacagtttga gataaataaa tttttgaaat |
| | atggacactg aaa |

Non-limiting examples of siRNA targeting SOX2 are described in, e.g., Kondo et al., Genes Develop. 18:2963-2972, 2004; Tani et al., J. Cancer Res. Clin. Oncol. 133(4):263-269, 2007; Chen et al., J. Biol. Chem. 283:17969-17978, 2008; and Card et al., Mol. Cell. Biol. 28(20):6426-6438, 2008. Non-limiting examples of shRNA targeting SOX2 are described in, e.g., Rudin et al., Nature Genetics 44:1111-1116, 2012; Basu-Roy et al., Oncogene 31:2270-2282, 2012; and Marques-Torrejon et al., Cell Stem Cell 12(1):88-100, 2013.

### Vectors

Some of the compositions provided herein can include at least two (e.g., two, three, four, five, or six) AAV vectors, where: each of the at least two different AAV vectors includes a coding sequence that encodes a differerent portion of a hair cell differentiation protein, each of the encoded portions being at least 30 amino acids (e.g., about 30 amino acids to about 800 amino acids, about 30 amino acids to about 780 amino acids, about 30 amino acids to about 760 amino acids, about 30 amino acids to about 750 amino acids, about 30 amino acids to about 740 amino acids, about 30 amino acids to about 720 amino acids, about 30 amino acids to about 710 amino acids, about 30 amino acids to about 700 amino acids, about 30 amino acids to about 690 amino acids, about 30 amino acids to about 680 amino acids, about 30 amino acids to about 670 amino acids, about 30 amino acids to about 660 amino acids, about 30 amino acids to about 650 amino acids, about 30 amino acids to about 640 amino acids, about 30 amino acids to about 630 amino acids, about 30 amino acids to about 620 amino acids, about 30 amino acids to about 610 amino acids, about 30 amino acids to about 600 amino acids, about 30 amino acids to about 590 amino acids, about 30 amino acids to about 580 amino acids, about 30 amino acids to about 570 amino acids, about 30 amino acids to about 560 amino acids, about 30 amino acids to about 550 amino acids, about 30 amino acids to about 540 amino acids, about 30 amino acids to about 530 amino acids, about 30 amino acids to about 520 amino acids, about 30 amino acids to about 510 amino acids, about 30 amino acids to about 500 amino acids, about 30 amino acids to about 490 amino acids, about 30 amino acids to about 480 amino acids, about 30 amino acids to about 470 amino acids, about 30 amino acids to about 460 amino acids, about 30 amino acids to about 450 amino acids, about 30 amino acids to about 440 amino acids, about 30 amino acids to about 430 amino acids, about 30 amino acids to about 420 amino acids, about 30 amino acids to about 410 amino acids, about 30 amino acids to about 400 amino acids, about 30 amino acids to about 390 amino acids, about 30 amino acids to about 380 amino acids, about 30 amino acids to about 370 amino acids, about 30 amino acids to about 360 amino acids, about 30 amino acids to about 350 amino acids, about 30 amino acids to about 340 amino acids, about 30 amino acids to about 330 amino acids, about 30 amino acids to about 320 amino acids, about 30 amino acids to about 310 amino acids, about 30 amino acids to about 300 amino acids, about 30 amino acids to about 290 amino acids, about 30 amino acids to about 280 amino acids, about 30 amino acids to about 270 amino acids, about 30 amino acids to about 260 amino acids, about 30 amino acids to about 250 amino acids, about 30 amino acids to about 240 amino acids, about 30 amino acids to about 230 amino acids, about 30 amino acids to about 220 amino acids, about 30 amino acids to about 210 amino acids, about 30 amino acids to about 200 amino acids, about 30 amino acids to about 190 amino acids, about 30 amino acids to about 180 amino acids, about 30 amino acids to about 170 amino acids, about 30 amino acids to about 160 amino acids, about 30 amino acids to about 150 amino acids, about 30 amino acids to about 140 amino acids, about 30 amino acids to about 140 amino acids, about 30 amino acids to about 130 amino acids, about 30 amino acids to about 120 amino acids, about 30 amino acids to about 110 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 50 amino acids to about 800 amino acids, about 50 amino acids to about 790 amino acids, about 50 amino acids to about 780 amino acids, about 50 amino acids to about 770 amino acids, about 50 amino acids to about 760 amino acids, about 50 amino acids to about 750 amino acids, about 50 amino acids to about 740 amino acids, about 50 amino acids to about 730 amino acids, about 50 amino acids to about 720 amino acids, about 50 amino acids to about 710 amino acids, about 50 amino acids to about 700 amino acids, about 50 amino acids to about 690 amino acids, about 50 amino acids to about 680 amino acids, about 50 amino acids to about 670 amino acids, about 50 amino acids to about 660 amino acids, about 50 amino acids to about 650 amino acids, about 50 amino acids to about 640 amino acids, about 50 amino acids to about 630 amino acids, about 50 amino acids to about 620 amino acids, about 50 amino acids to about 610 amino acids, about 50 amino acids to about 600 amino acids, about 50 amino acids to about 590 amino acids, about 50 amino acids to about 580 amino acids, about 50 amino acids to about 570 amino acids, about 50 amino acids to about 560 amino acids, about 50 amino acids to about 550 amino acids, about 50 amino acids to about 540 amino acids, about 50 amino acids to about 530 amino acids, about 50 amino acids to about 520 amino acids, about 50 amino acids to about 510 amino acids, about 50 amino acids to about 500 amino acids, about 50 amino acids to about 490 amino acids, about 50 amino acids to about 480 amino acids, about 50 amino acids to about 470 amino acids, about 50 amino acids to about 460 amino acids, about 50 amino acids to about 450 amino acids, about 50 amino acids to about 440 amino acids, about 50 amino acids to about 430 amino acids, about 50 amino acids to about 420 amino acids, about 50 amino acids to about 410 amino acids, about 50 amino acids to about 400 amino acids, about 50 amino acids to about 390 amino acids, about 50 amino acids to about 380 amino acids, about 50 amino acids to about 370 amino acids, about 50 amino acids to about 360 amino acids, about 50 amino acids to about 350 amino acids, about 50 amino acids to about 340 amino acids, about 50 amino acids to about 330 amino acids, about 50 amino acids to about 320 amino acids, about 50 amino acids to about 310 amino acids, about 50 amino acids to about 300 amino acids, about 50 amino acids to about 290 amino acids, about 50 amino acids to about 280 amino acids, about 50 amino acids to about 270 amino acids, about 50 amino acids to about 260 amino acids, about 50 amino acids to about 250 amino acids, about 50 amino acids to about 240 amino acids, about 50 amino acids to about 230 amino acids, about 50 amino acids to about 220 amino acids, about 50 amino acids to about 210 amino acids, about 50 amino acids to about 200 amino acids, about 50 amino acids to about 190 amino acids, about 50 amino acids to about 180 amino acids, about 50 amino acids to about 170 amino acids, about 50 amino acids to about 160 amino acids, about 50 amino acids to about 150 amino acids, about 50 amino acids to about 140 amino acids, about 50 amino acids to about 130 amino acids, about 50 amino acids to about 120 amino acids, about 50 amino acids to about 110 amino acids, about 50 amino acids to about 100 amino acids, about 100 amino acids to about 800 amino acids, about 100 amino acids to about 790 amino acids, about 100 amino acids to about 780 amino acids, about 100 amino acids to about 770 amino acids, about 100 amino acids to about 760 amino acids, about 100 amino acids to about 750 amino acids, about 100 amino acids to about 740 amino acids, about 100 amino acids to about 730 amino acids, about 100 amino acids to about 720 amino acids, about 100 amino acids to about 710 amino acids, about 100 amino acids to about 700 amino acids, about 100 amino acids to about 690 amino acids, about 100 amino acids to, about 680 amino acids, about 100 amino acids to about 670 amino acids, about 100 amino acids to about 660 amino acids, about 100 amino acids to about 650 amino acids, about 100 amino acids to about 640 amino acids, about 100 amino acids to about 630 amino acids, about 100 amino acids to about 620 amino acids, about 100 amino acids to about 610 amino acids, about 100 amino acids to about 600 amino acids, about 100 amino acids to about 590 amino acids, about 100 amino acids to about 580 amino acids, about 100 amino acids to about 570 amino acids, about 100 amino acids to about 560 amino acids, about 100 amino acids to about 550 amino acids, about 100 amino acids to about 540 amino acids, about 100 amino acids to about 530 amino acids, about 100 amino acids to about 520 amino acids, about 100 amino acids to about 510 amino acids, about 100 amino acids to about 500 amino acids, about 100 amino acids to about 490 amino acids, about 100 amino acids to about 480 amino acids, about 100 amino acids to about 470 amino acids, about 100 amino acids to about 460 amino acids, about 100 amino acids to about 450 amino acids, about 100 amino acids to about 440 amino acids, about 100 amino acids to about 430 amino acids, about 100 amino acids to about 420 amino acids, about 100 amino acids to about 410 amino acids, about 100 amino acids to about 400 amino acids, about 100 amino acids to about 390 amino acids, about 100 amino acids to about 380 amino acids, about 100 amino acids to about 370 amino acids, about 100 amino acids to about 360 amino acids, about 100 amino acids to about 350 amino acids, about 100 amino acids to about 340 amino acids, about 100 amino acids to about 330 amino acids, about 100 amino acids to about 320 amino acids, about 100 amino acids to about 310 amino acids, about 100 amino acids to about 300 amino acids, about 100 amino acids to about 290 amino acids, about 100 amino acids to about 280 amino acids, about 100 amino acids to about 270 amino acids, about 100 amino acids to about 260 amino acids, about 100 amino acids to about 250 amino acids, about 100 amino acids to about 240 amino acids, about 100 amino acids to about 230 amino acids, about 100 amino acids to about 220 amino acids, about 100 amino acids to about 210 amino acids, about 100 amino acids to about 200 amino acids, about 100 amino acids to about 190 amino acids, about 100 amino acids to about 180 amino acids, about 100 amino acids to about 170 amino acids, about 100 amino acids to about 160 amino acids, about 100 amino acids to about 150 amino acids, about 150 amino acids to about 800 amino acids, about 150 amino acids to about 790 amino acids, about 150 amino acids to about 780 amino acids, about 150 amino acids to about 770 amino acids, about 150 amino acids to about 760 amino acids, about 150 amino acids to about 750 amino acids, about 150 amino acids to about 740 amino acids, about 150 amino acids to about 730 amino acids, about 150 amino acids to about 720 amino acids, about 150 amino acids to about 710 amino acids, about 150 amino acids to about 700 amino acids, about 150 amino acids to about 690 amino acids, about 150 amino acids to about 680 amino acids, about 150 amino acids to about 670 amino acids, about 150 amino acids to about 660 amino acids, about 150 amino acids to about 650 amino acids, about 150 amino acids to about 640 amino acids, about 150 amino acids to about 630 amino acids, about 150 amino acids to about 620 amino acids, about 150 amino acids to about 610 amino acids, about 150 amino acids to about 600 amino acids, about 150 amino acids to about 590 amino acids, about 150 amino acids to about 580 amino acids, about 150 amino acids to about 570 amino acids, about 150 amino acids to about 560 amino acids, about 150 amino acids to about 550 amino acids, about 150 amino acids to about 540 amino acids, about 150 amino acids to about 530 amino acids, about 150 amino acids to about 520 amino acids, about 150 amino acids to about 510 amino acids, about 150 amino acids to about 500 amino acids, about 150 amino acids to about 490 amino acids, about 150 amino acids to about 480 amino acids, about 150 amino acids to about 470 amino acid's, about 150 amino acids to about 460 amino acids, about 150 amino acids to about 450 amino acids, about 150 amino acids to about 440 amino acids, about 150 amino acids to about 430 amino acids, about 150 amino acids to about 420 amino acids, about 150 amino acids to about 410 amino acids, about 150 amino acids to about 400 amino acids, about 150 amino acids to about 390 amino acids, about 150 amino acids to about 380 amino acids, about 150 amino acids to about 370 amino acids, about 150 amino acids to about 360 amino acids, about 150 amino acids to about 350 amino acids, about 150 amino acids to about 340 amino acids, about 150 amino acids to about 330 amino acids, about 150 amino acids to about 320 amino acids, about 150 amino acids to about 310 amino acids, about 150 amino acids to about 300 amino acids, about 150 amino acids to about 290 amino acids, about 150 amino acids to about 280 amino acids, about 150 amino acids to about 270 amino acids, about 150 amino acids to about 260 amino acids, about 150 amino acids to about 250 amino acids, about 150 amino acids to about 240 amino acids, about 150 amino acids to about 230 amino acids, about 150 amino acids to about 220 amino acids, about 150 amino acids to about 210 amino acids, about 150 amino acids to about 200 amino acids, about 200 amino acids to about 800 amino acids, about 200 amino acids to about 790 amino acids, about 200 amino acids to about 780 amino acids, about 200 amino acids to about 770 amino acids, about 200 amino acids to about 760 amino acids, about 200 amino acids to about 750 amino acids, about 200 amino acids to about 740 amino acids, about 200 amino acids to about 730 amino acids, about 200 amino acids to about 720 amino acids, about 200 amino acids to about 710 amino acids, about 200 amino acids to about 700 amino acids, about 200 amino acids to about 690 amino acids, about 200 amino acids to about 680 amino acids, about 200 amino acids to about 670 amino acids, about 200 amino acids to about 660 amino acids, about 200 amino acids to about 650 amino acids, about 200 amino acids to about 640 amino acids, about 200 amino acids to about 630 amino acids, about 200 amino acids to about 620 amino acids, about 200 amino acids to about 610 amino acids, about 200 amino acids to about 600 amino acids, about 200 amino acids to about 590 amino acids, about 200 amino acids to about 580 amino acids, about 200 amino acids to about 570 amino acids, about 200 amino acids to about 560 amino acids, about 200 amino acids to about 550 amino acids, about 200 amino acids to about 540 amino acids, about 200 amino acids to about 530 amino acids, about 200 amino acids to about 520 amino acids, about 200 amino acids to about 510 amino acids, about 200 amino acids to about 500 amino acids, about 200 amino acids to about 490 amino acids, about 200 amino acids to about 480 amino acids, about 200 amino acids to about 470 amino acids, about 200 amino acids to about 460 amino acids, about 200 amino acids to about 450 amino acids, about 200 amino acids to about 440 amino acids, about 200 amino acids to about 430 amino acids, about 200 amino acids to about 420 amino acids, about 200 amino acids to about 410 amino acids, about 200 amino acids to about 400 amino acids, about 200 amino acids to about 390 amino acids, about 200 amino acids to about 380 amino acids, about 200 amino acids to about 370 amino acids, about 200 amino acids to about 360 amino acids, about 200 amino acids to about 350 amino acids, about 200 amino acids to about 340 amino acids, about 200 amino acids to about 330 amino acids, about 200 amino acids to about 320 amino acids, about 200 amino acids to about 310 amino acids, about 200 amino acids to about 300 amino acids, about 200 amino acids to about 290 amino acids, about 200 amino acids to about 280 amino acids, about 200 amino acids to about 270 amino acids, about 200 amino acids to about 260 amino acids, about 200 amino acids to about 250 amino acids, about 250 amino acids to about 800 amino acids, about 250 amino acids to about 790 amino acids, about 250 amino acids to about 780 amino acids, about 250 amino acids to about 770 amino acids, about 250 amino acids to about 760 amino acids, about 250 amino acids to about 750 amino acids, about 250 amino acids to about 740 amino acids, about 250 amino acids to about 730 amino acids, about 250 amino acids to about 720 amino acids, about 250 amino acids to about 710 amino acids, about 250 amino acids to about 700 amino acids, about 250 amino acids to about 690 amino acids, about 250 amino acids to about 680 amino acids, about 250 amino acids to about 670 amino acids, about 250 amino acids to about 660 amino acids, about 250 amino acids to about 650 amino acids, about 250 amino acids to about 640 amino acids, about 250 amino acids to about 630 amino acids, about 250 amino acids to about 620 amino acids, about 250 amino acids to about 610 amino acids, about 250 amino acids to about 600 amino acids, about 250 amino acids to about 590 amino acids, about 250 amino acids to about 580 amino acids, about 250 amino acids to about 570 amino acids, about 250 amino acids to about 560 amino acids, about 250 amino acids to about 550 amino acids, about 250 amino acids to about 540 amino acids, about 250 amino acids to about 530 amino acids, about 250 amino acids to about 520 amino acids, about 250 amino acids to about 510 amino acids, about 250 amino acids to about 500 amino acids, about 250 amino acids to about 490 amino acids, about 250 amino acids to about 480 amino acids, about 250 amino acids to about 470 amino acids, about 250 amino acids to about 460 amino acids, about 250 amino acids to about 450 amino acids, about 250 amino acids to about 440 amino acids, about 250 amino acids to about 430 amino acids, about 250 amino acids to about 420 amino acids, about 250 amino acids to about 410 amino acids, about 250 amino acids to about 400 amino acids, about 250 amino acids to about 390 amino acids, about 250 amino acids to about 380 amino acids, about 250 amino acids to about 370 amino acids, about 250 amino acids to about 360 amino acids, about 250 amino acids to about 350 amino acids, about 250 amino acids to about 340 amino acids, about 250 amino acids to about 330 amino acids, about 250 amino acids to about 320 amino acids, about 250 amino acids to about 310 amino acids, about 250 amino acids to about 300 amino acids, about 300 amino acids to about 800 amino acids, about 300 amino acids to about 790 amino acids, about 300 amino acids to about 780 amino acids, about 300 amino acids to about 770 amino acids, about 300 amino acids to about 760 amino acids, about 300 amino acids to about 750 amino acids, about 300 amino acids to about 740 amino acids, about 300 amino acids to about 730 amino acids, about 300 amino acids to about 720 amino acids, about 300 amino acids to about 710 amino acids, about 300 amino acids to about 700 amino acids, about 300 amino acids to about 690 amino acids, about 300 amino acids to about 680 amino acids, about 300 amino acids to about 670 amino acids, about 300 amino acids to about 660 amino acids, about 300 amino acids to about 650 amino acids, about 300 amino acids to about 640 amino acids, about 300 amino acids to about 630 amino acids, about 300 amino acids to about 620 amino acids, about 300 amino acids to about 610 amino acids, about 300 amino acids to about 600 amino acids, about 300 amino acids to about 590 amino acids, about 300 amino acids to about 580 amino acids, about 300 amino acids to about 570 amino acids, about 300 amino acids to about 560 amino acids, about 300 amino acids to about 550 amino acids, about 300 amino acids to about 540 amino acids, about 300 amino acids to about 530 amino acids, about 300 amino acids to about 520 amino acids, about 300 amino acids to about 510 amino acids, about 300 amino acids to about 500 amino acids, about 300 amino acids to about 490 amino acids, about 300 amino acids to about 480 amino acids, about 300 amino acids to about 470 amino acids, about 300 amino acids to about 460 amino acids, about 300 amino acids to about 450 amino acids, about 300 amino acids to about 440 amino acids, about 300 amino acids to about 430 amino acids, about 300 amino acids to about 420 amino acids, about 300 amino acids to about 410 amino acids, about 300 amino acids to about 400 amino acids, about 300 amino acids to about 390 amino acids, about 300 amino acids to about 380 amino acids, about 300 amino acids to about 370 amino acids, about 300 amino acids to about 360 amino acids, about 300 amino acids to about 350 amino acids, about 350 amino acids to about 800 amino acids, about 350 amino acids to about 790 amino acids, about 350 amino acids to about 780 amino acids, about 350 amino acids to about 770 amino acids, about 350 amino acids to about 760 amino acids, about 350 amino acids to about 750 amino acids, about 350 amino acids to about 740 amino acids, about 350 amino acids to about 730 amino acids, about 350 amino acids to about 720 amino acids, about 350 amino acids to about 710 amino acids, about 350 amino acids to about 700 amino acids, about 350 amino acids to about 690 amino acids, about 350 amino acids to about 680 amino acids, about 350 amino acids to about 670 amino acids, about 350 amino acids to about 660 amino acids, about 350 amino acids to about 650 amino acids, about 350 amino acids to about 640 amino acids, about 350 amino acids to about 630 amino acids, about 350 amino acids to about 620 amino acids, about 350 amino acids to about 610 amino acids, about 350 amino acids to about 600 amino acids, about 350 amino acids to about 590 amino acids, about 350 amino acids to about 580 amino acids, about 350 amino acids to about 570 amino acids, about 350 amino acids to about 560 amino acids, about 350 amino acids to about 550 amino acids, about 350 amino acids to about 540 amino acids, about 350 amino acids to about 530 amino acids, about 350 amino acids to about 520 amino acids, about 350 amino acids to about 510 amino acids, about 350 amino acids to about 500 amino acids, about 350 amino acids to about 490 amino acids, about 350 amino acids to about 480 amino acids, about 350 amino acids to about 470 amino acids, about 350 amino acids to about 460 amino acids, about 350 amino acids to about 450 amino acids, about 350 amino acids to about 440 amino acids, about 350 amino acids to about 430 amino acids, about 350 amino acids to about 420 amino acids, about 350 amino acids to about 410 amino acids, about 350 amino acids to about 400 amino acids, about 400 amino acids to about 800 amino acids, about 400 amino acids to about 790 amino acids, about 400 amino acids to about 780 amino acids, about 400 amino acids to about 770 amino acids, about 400 amino acids to about 760 amino acids, about 400 amino acids to about 750 amino acids, about 400 amino acids to about 740 amino acids, about 400 amino acids to about 730 amino acids, about 400 amino acids to about 720 amino acids, about 400 amino acids to about 710 amino acids, about 400 amino acids to about 700 amino acids, about 400 amino acids to about 690 amino acids, about 400 amino acids to about 680 amino acids, about 400 amino acids to about 670 amino acids, about 400 amino acids to about 660 amino acids, about 400 amino acids to about 650 amino acids, about 400 amino acids to about 640 amino acids, about 400 amino acids to about 630 amino acids, about 400 amino acids to about 620 amino acids, about 400 amino acids to about 610 amino acids, about 400 amino acids to about 600 amino acids, about 400 amino acids to about 590 amino acids, about 400 amino acids to about 580 amino acids, about 400 amino acids to about 570 amino acids, about 400 amino acids to about 560 amino acids, about 400 amino acids to about 550 amino acids, about 400 amino acids to about 540 amino acids, about 400 amino acids to about 530 amino acids, about 400 amino acids to about 520 amino acids, about 400 amino acids to about 510 amino acids, about 400 amino acids to about 500 amino acids, about 400 amino acids to about 490 amino acids, about 400 amino acids to about 480 amino acids, about 400 amino acids to about 470 amino acids, about 400 amino acids to about 460 amino acids, about 400 amino acids to about 450 amino acids, about 400 amino acids to about 440 amino acids, about 400 amino acids to about 430 amino acids, about 400 amino acids to about 420 amino acids, about 400 amino acids to about 410 amino acids, about 450 amino acids to about 800 amino acids, about 450 amino acids to about 790 amino acids, about 450 amino acids to about 780 amino acids, about 450 amino acids to about 770 amino acids, about 450 amino acids to about 760 amino acids, about 450 amino acids to about 750 amino acids, about 450 amino acids to about 740 amino acids, about 450 amino acids to about 730 amino acids, about 450 amino acids to about 720 amino acids, about 450 amino acids to about 710 amino acids, about 450 amino acids to about 700 amino acids, about 450 amino acids to about 690 amino acids, about 450 amino acids to about 680 amino acids, about 450 amino acids to about 670 amino acids, about 450 amino acids to about 660 amino acids, about 450 amino acids to about 650 amino acids, about 450 amino acids to about 640 amino acids, about 450 amino acids to about 630 amino acids, about 450 amino acids to about 620 amino acids, about 450 amino acids to about 610 amino acids, about 450 amino acids to about 600 amino acids, about 450 amino acids to about 590 amino acids, about 450 amino acids to about 580 amino acids, about 450 amino acids to about 570 amino acids, about 450 amino acids to about 560 amino acids, about 450 amino acids to about 550 amino acids, about 450 amino acids to about 540 amino acids, about 450 amino acids to about 530 amino acids, about 450 amino acids to about 520 amino acids, about 450 amino acids to about 510 amino acids, about 450 amino acids to about 500 amino acids, about 500 amino acids to about 800 amino acids, about 500 amino acids to about 790 amino acids, about 500 amino acids to about 780 amino acids, about 500 amino acids to about 770 amino acids, about 500 amino acids to about 760 amino acids, about 500 amino acids to about 750 amino acids, about 500 amino acids to about 740 amino acids, about 500 amino acids to about 730 amino acids, about 500 amino acids to about 720 amino acids, about 500 amino acids to about 710 amino acids, about 500 amino acids to about 700 amino acids, about 500 amino acids to about 690 amino acids, about 500 amino acids to about 680 amino acids, about 500 amino acids to about 670 amino acids, about 500 amino acids to about 660 amino acids, about 500 amino acids to about 650 amino acids, about 500 amino acids to about 640 amino acids, about 500 amino acids to about 630 amino acids, about 500 amino acids to about 620 amino acids, about 500 amino acids to about 610 amino acids, about 500 amino acids to about 600 amino acids, about 500 amino acids to about 590 amino acids, about 500 amino acids to about 580 amino acids, about 500 amino acids to about 570 amino acids, about 500 amino acids to about 560 amino acids, about 500 amino acids to about 550 amino acids, about 550 amino acids to about 800 amino acids, about 550 amino acids to about 790 amino acids, about 550 amino acids to about 780 amino acids, about 550 amino acids to about 770 amino acids, about 550 amino acids to about 760 amino acids, about 550 amino acids to about 750 amino acids, about 550 amino acids to about 740 amino acids, about 550 amino acids to about 730 amino acids, about 550 amino acids to about 720 amino acids, about 550 amino acids to about 710 amino acids, about 550 amino acids to about 700 amino acids, about 550 amino acids to about 690 amino acids, about 550 amino acids to about 680 amino acids, about 550 amino acids to about 670 amino acids, about 550 amino acids to about 660 amino acids, about 550 amino acids to about 650 amino acids, about 550 amino acids to about 640 amino acids, about 550 amino acids to about 780 amino acids, about 550 amino acids to about 630 amino acids, about 550 amino acids to about 620 amino acids, about 550 amino acids to about 600 amino acids, about 600 amino acids to about 800 amino acids, about 600 amino acids to about 790 amino acids, about 600 amino acids to about 780 amino acids, about 600 amino acids to about 770 amino acids, about 600 amino acids to about 760 amino acids, about 600 amino acids to about 750 amino acids, about 600 amino acids to about 740 amino acids, about 600 amino acids to about 730 amino acids, about 600 amino acids to about 720 amino acids, about 600 amino acids to about 710 amino acids, about 600 amino acids to about 700 amino acids, about 550 amino acids to about 690 amino acids, about 550 amino acids to about 680 amino acids, about 550 amino acids to about 670 amino acids, about 550 amino acids to about 660 amino acids, about 600 amino acids to about 650 amino acids, about 650 amino acids to about 800 amino acids, about 650 amino acids to about 790 amino acids, about 650 amino acids to about 780 amino acids, about 650 amino acids to about 770 amino acids, about 650 amino acids to about 760 amino acids, about 650 amino acids to about 750 amino acids, about 650 amino acids to about 740 amino acids, about 650 amino acids to about 730 amino acids, about 650 amino acids to about 720 amino acids, about 650 amino acids to about 710 amino acids, about 650 amino acids to about 700 amino acids, about 700 amino acids to about 800 amino acids, about 700 amino acids to about 790 amino acids, about 700 amino acids to about 780 amino acids, about 700 amino acids to about 770 amino acids, about 700 amino acids to about 760 amino acids, about 700 amino acids to about 750 amino acids, or about 750 amino acids to about 800 amino acids), where the amino acid sequence of each of the encoded portions may optionally partially overlap with the amino acid sequence of a different one of the encoded portions; no single vector of the at least two different vectors encodes the hair cell differentiation protein (e.g., a full-length hair cell differentiation protein (e.g., a full-length wildtype hair cell differentiation protein)); and, when introduced into a primate cell (e.g., a hair cell or a supporting cell of the inner ear), the at least two different AAV vectors undergo homologous recombination with each other, where the recombined nucleic acid encodes a hair cell differentiation protein (e.g., a full-length hair cell differentiation protein).

In some embodiments of the compositions that include at least two AAV vectors, at least one of the coding sequences includes a nucleotide sequence spanning two neighboring exons of hair cell differentiation genomic DNA, and lacks the intronic sequence that naturally occurs between the two neighboring exons.

In some embodiments of the compositions that include at least two AAV vectors, the amino acid sequence of none of the encoded portions overlaps even in part with the amino acid sequence of a different one of the encoded portions. In some embodiments of the compositions that include at least two AAV vectors, the amino acid sequence of one or more of the encoded portions partially overlaps with the amino acid sequence of a different one of the encoded portions. In some embodiments of the compositions that include at least AAV vectors, the amino acid sequence of each of the encoded portions partially overlaps with the amino acid sequence of a different one of the encoded portions.

In some embodiments of the compositions that include at least two AAV vectors, the overlapping amino acid sequence is between about 30 amino acid residues to about 800 amino acids (e.g., or any of the subranges of this range described herein) in length.

In some examples, the compositions include two different AAV vectors, each of which comprises a different segment of an intron, where the intron includes the nucleotide sequence of an intron that is present in a hair cell differentiation genomic DNA, and where the two different segments overlap in sequence by at least 100 nucleotides (e.g., about 100 nucleotides to about 3,000 nucleotides, about 100 nucleotides to about 2,500 nucleotides, about 100 nucleotides to about 2,000 nucleotides, about 100 nucleotides to about 1,500 nucleotides, about 100 nucleotides to about 1,000 nucleotides, about 100 nucleotides to about 800 nucleotides, about 100 nucleotides to about 600 nucleotides, about 100 nucleotides to about 400 nucleotides, about 100 nucleotides to about 200 nucleotides, about 200 nucleotides to about 3,000 nucleotides, about 200 nucleotides to about 2,500 nucleotides, about 200 nucleotides to about 2,000 nucleotides, about 200 nucleotides to about 1,500 nucleotides, about 200 nucleotides to about 1,000 nucleotides, about 200 nucleotides to about 800 nucleotides, about 200 nucleotides to about 600 nucleotides, about 200 nucleotides to about 400 nucleotides about 400 nucleotides to about 3,000 nucleotides, about 400 nucleotides to about 2,500 nucleotides, about 400 nucleotides to about 2,000 nucleotides, about 400 nucleotides to about 1,500 nucleotides, about 400 nucleotides to about 1,000 nucleotides, about 400 nucleotides to about 800 nucleotides, about 400 nucleotides to about 600 nucleotides, about 600 nucleotides to about 3,000 nucleotides, about 600 nucleotides to about 2,500 nucleotides, about 600 nucleotides to about 2,000 nucleotides, about 600 nucleotides to about 1,500 nucleotides, about 600 nucleotides to about 1,000 nucleotides, about 600 nucleotides to about 800 nucleotides, about 800 nucleotides to about 3,000 nucleotides, about 800 nucleotides to about 2,500 nucleotides, about 800 nucleotides to about 2,000 nucleotides, about 800 nucleotides to about 1,500 nucleotides, about 800 nucleotides to about 1,000 nucleotides, about 1,000 nucleotides to about 3,000 nucleotides, about 1,000 nucleotides to about 2,500 nucleotides, about 1,000 nucleotides to about 2,000 nucleotides, about 1,000 nucleotides to about 1,500 nucleotides, about 1,500 nucleotides to about 3,000 nucleotides, about 1,500 nucleotides to about 2,500 nucleotides, about 1,500 nucleotides to about 2,000 nucleotides, about 2,000 nucleotides to about 3,000 nucleotides, about 2,000 nucleotides to about 2,500 nucleotides, or about 2,500 nucleotides to about 3,000 nucleotides), in length.

The overlapping nucleotide sequence in any two of the different AAV vectors can include part or all of one or more exons of a hair cell differentiation gene.

In some embodiments, the number of different AAV vectors in the composition is two, three, four, or five. In compositions where the number of different AAV vectors in the composition is two, the first of the two different vectors can include a coding sequence that encodes an N-terminal portion of the hair cell differentiation protein. In some embodiments, the N-terminal portion can include a portion having about 30 amino acids to about 800 amino acids (or any of the subranges of this range described herein). In some examples, the N-terminal portion encoded by one of the two vectors can include a portion comprising amino acid position 1 to about amino acid position 800, about amino acid position 790, about amino acid position 780, about amino acid position 770, about amino acid position 760, about amino acid position 750, about amino acid position 740, about amino acid position 730, about amino acid position 720, about amino acid position 710, about amino acid position 700, about amino acid position 690, about amino acid position 680, about amino acid position 670, about amino acid position 660, about amino acid position 650, about amino acid position 640, about amino acid position 630, about amino acid position 620, about amino acid position 610, about amino acid position 600, about amino acid position 590, about amino acid position 580, about amino acid position 570, about amino acid position 560, about amino acid position 550, about amino acid position 540, about amino acid position 530, about amino acid position 520, about amino acid position 510, about amino acid position 500, about amino acid position 490, about amino acid position 480, about amino acid position 470, about amino acid position 460, about amino acid position 450, about amino acid position 440, about amino acid position 430, about amino acid position 420, about amino acid position 410, about amino acid position 400, about amino acid position 390, about amino acid position 380, about amino acid position 370, about amino acid position 360, about amino acid position 350, about amino acid position 340, about amino acid position 330, about amino acid position 320, about amino acid position 310, about amino acid position 300, about amino acid position 290, about amino acid position 280, about amino acid position 270, about amino acid position 260, about amino acid position 250, about amino acid position 240, about amino acid position 230, about amino acid position 220, about amino acid position 210, about amino acid position 200, about amino acid position 190, about amino acid position 180, about amino acid position 170, about amino acid position 160, about amino acid position 150, about amino acid position 140, about amino acid position 130, about amino acid position 120, about amino acid position 110, about amino acid position 100, about amino acid position 90, about amino acid position 80, about amino acid position 70, about amino acid position 60, about amino acid position 50, or about amino acid position 40 of a wildtype hair cell differentiation protein.

In compositions where the number of different AAV vectors in the composition is two, the second of the two different vectors can include a coding sequence that encodes a C-terminal portion of the hair cell differentiation protein. In some embodiments, the C-terminal portion can include a portion having about 30 amino acids to about 800 amino acids (or any of the subranges of this range described herein).

As used herein, the term “vector” means a composition including a polynucleotide capable of carrying at least one exogenous nucleic acid fragment, e.g., an adeno-associated virus (AAV) vector. A vector can, e.g., include sufficient cis-acting elements for expression; other elements for expression can be supplied by the host primate cell or in an in vitro expression system. The term “vector” includes any genetic element (e.g., a plasmid, a transposon, a cosmid, an artificial chromosome, or a viral vector, etc.) that is capable of replicating when associated with the proper control elements.

“Recombinant AAV vectors” or “rAAVs” are typically composed of, at a minimum, a transgene or a portion thereof and a regulatory sequence, and optionally 5′ and 3′ AAV inverted terminal repeats (ITRs). Such a recombinant AAV vector is packaged into a capsid and delivered to a selected target cell (e.g., an inner or outer hair cell, or a supporting cell of the inner ear).

The AAV sequences of the vector typically comprise the cis-acting 5′ and 3′ ITR sequences (See, e.g., B. J. Carter, in “Handbook of Parvoviruses”, ed., P. Tijsser, CRC Press, pp. 155 168, 1990). Typical AAV ITR sequences are about 145 nucleotides in length. In some embodiments, at least 75% of a typical ITR sequence (e.g., at least 80%, at least 85%, at least 90%, or at least 95%) is incorporated into the AAV vector. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996). In some embodiments, any of the coding sequences described herein are flanked by 5′ and 3′ AAV ITR sequences in the AAV vectors. The AAV ITR sequences may be obtained from any known AAV, including presently identified AAV types. In some examples of any of the vectors described herein, the vector includes a 5′ ITR sequence

(SEQ ID NO: 51)
CCTGCAGGCAGCTGCGCGCTCGCTCGCTCACTGAGGCCGCCCGGGCGTCG
CGGCGACTTTGGTCGCCCGGCCTCAGTGAGCGAGCGAGCGCGCAGAGAGG
GAGTGGCCAACTCCATCACTAGGGGTTCCT
and/or
a 3′ ITR sequence
(SEQ ID NO: 57)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG.

AAV vectors as described herein may include any of the regulatory elements described herein (e.g., one or more of a promoter, a polyA sequence, and an IRES).

In some embodiments, the AAV vector is selected from the group consisting of: an AAV1 vector, an AAV2 vector, an AAV3 vector, an AAV4 vector, an AAV5 vector, an AAV6 vector, an AAV7 vector, an AAV8 vector, an AAV9 vector, an AAV2.7m8 vector, an AAV8BP2 vector, and an AAV293 vector. Additional exemplary AAV vectors that can be used herein are known in the art. See, e.g., Kanaan et al., Mol. Ther. Nucleic Acids 8:184-197, 2017; Li et al., Mol. Ther. 16(7): 1252-1260; Adachi et al., Nat. Commun. 5: 3075, 2014; Isgrig et al., Nat. Commun. 10(1): 427, 2019; and Gao et al., J. Virol. 78(12): 6381-6388.

In some embodiments, an AAV vector provided herein includes or consists of a sequence that is at least 80% identical (e.g., at least 82%, at least 84%, at least 85%, at least 86%, at least 88%, at least 90%, at least 92%, at least 94%, at least 95%, at least 96%, at least 98%, at least 99%, or 100% identical) to SEQ ID NO: 50, 58, 60, 64, 66, 68, 78, 79, 81, 82, 83 or 94.

The AAV vectors provided herein can be of different sizes. In some embodiments, the AAV vector(s) can include a total number of nucleotides of up to 5 kb. In some embodiments, the AAV vector(s) can include a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

In some embodiments of any of the compositions, kits, and methods provided herein, the at least two different AAV vectors can be substantially the same type of vector and may differ in size. In some embodiments, the at least two different AAV vectors can be different types of AAV vector, and may have substantially the same size or have different sizes.

In some embodiments, any of the at least two AAV vectors can have a total number of nucleotides in the range of about 500 nucleotides to about 10,000 nucleotides, about 500 nucleotides to about 9,500 nucleotides, about 500 nucleotides to about 9,000 nucleotides, about 500 nucleotides to about 8,500 nucleotides, about 500 nucleotides to about 8,000 nucleotides, about 500 nucleotides to about 7,800 nucleotides, about 500 nucleotides to about 7,600 nucleotides, about 500 nucleotides to about 7,400 nucleotides, about 500 nucleotides to about 7,200 nucleotides, about 500 nucleotides to about 7,000 nucleotides, about 500 nucleotides to about 6,800 nucleotides, about 500 nucleotides to about 6,600 nucleotides, about 500 nucleotides to about 6,400 nucleotides, about 500 nucleotides to about 6,200 nucleotides, about 500 nucleotides to about 6,000 nucleotides, about 500 nucleotides to about 5,800 nucleotides, about 500 nucleotides to about 5,600 nucleotides, about 500 nucleotides to about 5,400 nucleotides, about 500 nucleotides to about 5,200 nucleotides, about 500 nucleotides to about 5,000 nucleotides, about 500 nucleotides to about 4,800 nucleotides, about 4,600 nucleotides, about 500 nucleotides to about 4,400 nucleotides, about 500 nucleotides to about 4,200 nucleotides, about 500 nucleotides to about 4,000 nucleotides, about 500 nucleotides to about 3,800 nucleotides, about 500 nucleotides to about 3,600 nucleotides, about 500 nucleotides to about 3,400 nucleotides, about 500 nucleotides to about 3,200 nucleotides, about 500 nucleotides to about 3,000 nucleotides, about 500 nucleotides to about 2,800 nucleotides, about 500 nucleotides to about 2,600 nucleotides, about 500 nucleotides to about 2,400 nucleotides, about 500 nucleotides to about 2,200 nucleotides, about 500 nucleotides to about 2,000 nucleotides, about 500 nucleotides to about 1,800 nucleotides, about 500 nucleotides to about 1,600 nucleotides, about 500 nucleotides to about 1,400 nucleotides, about 500 nucleotides to about 1,200 nucleotides, about 500 nucleotides to about 1,000 nucleotides, about 500 nucleotides to about 800 nucleotides, about 800 nucleotides to about 10,000 nucleotides, about 800 nucleotides to about 9,500 nucleotides, about 800 nucleotides to about 9,000 nucleotides, about 800 nucleotides to about 8,500 nucleotides, about 800 nucleotides to about 8,000 nucleotides, about 800 nucleotides to about 7,800 nucleotides, about 800 nucleotides to about 7,600 nucleotides, about 800 nucleotides to about 7,400 nucleotides, about 800 nucleotides to about 7,200 nucleotides, about 800 nucleotides to about 7,000 nucleotides, about 800 nucleotides to about 6,800 nucleotides, about 800 nucleotides to about 6,600 nucleotides, about 800 nucleotides to about 6,400 nucleotides, about 800 nucleotides to about 6,200 nucleotides, about 800 nucleotides to about 6,000 nucleotides, about 800 nucleotides to about 5,800 nucleotides, about 800 nucleotides to about 5,600 nucleotides, about 800 nucleotides to about 5,400 nucleotides, about 800 nucleotides to about 5,200 nucleotides, about 800 nucleotides to about 5,000 nucleotides, about 800 nucleotides to about 4,800 nucleotides, about 800 nucleotides to about 4,600 nucleotides, about 800 nucleotides to about 4,400 nucleotides, about 800 nucleotides to about 4,200 nucleotides, about 800 nucleotides to about 4,000 nucleotides, about 800 nucleotides to about 3,800 nucleotides, about 800 nucleotides to about 3,600 nucleotides, about 800 nucleotides to about 3,400 nucleotides, about 800 nucleotides to about 3,200 nucleotides, about 800 nucleotides to about 3,000 nucleotides, about 800 nucleotides to about 2,800 nucleotides, about 800 nucleotides to about 2,600 nucleotides, about 800 nucleotides to about 2,400 nucleotides, about 800 nucleotides to about 2,200 nucleotides, about 800 nucleotides to about 2,000 nucleotides, about 800 nucleotides to about 1,800 nucleotides, about 800 nucleotides to about 1,600 nucleotides, about 800 nucleotides to about 1,400 nucleotides, about 800 nucleotides to about 1,200 nucleotides, about 800 nucleotides to about 1,000 nucleotides, about 1,000 nucleotides to about 10,000 nucleotides, about 1,000 nucleotides to about 9,000 nucleotides, about 1,000 nucleotides to about 8,500 nucleotides, about 1,000 nucleotides to about 8,000 nucleotides, about 1,000 nucleotides to about 7,800 nucleotides, about 1,000 nucleotides to about 7,600 nucleotides, about 1,000 nucleotides to about 7,400 nucleotides, about 1,000 nucleotides to about 7,200 nucleotides, about 1,000 nucleotides to about 7,000 nucleotides, about 1,000 nucleotides to about 6,800 nucleotides, about 1,000 nucleotides to about 6,600 nucleotides, about 1,000 nucleotides to about 6,400 nucleotides, about 1,000 nucleotides to about 6,200 nucleotides, about 1,000 nucleotides to about 6,000 nucleotides, about 1,000 nucleotides to about 5,800 nucleotides, about 1,000 nucleotides to about 5,600 nucleotides, about 1,000 nucleotides to about 5,400 nucleotides, about 1,000 nucleotides to about 5,200 nucleotides, about 1,000 nucleotides to about 5,000 nucleotides, about 1,000 nucleotides to about 4,800 nucleotides, about 1,000 nucleotides to about 4,600 nucleotides, about 1,000 nucleotides to about 4,400 nucleotides, about 1,000 nucleotides to about 4,200 nucleotides, about 1,000 nucleotides to about 4,000 nucleotides, about 1,000 nucleotides to about 3,800 nucleotides, about 1,000 nucleotides to about 3,600 nucleotides, about 1,000 nucleotides to about 3,400 nucleotides, about 1,000 nucleotides to about 3,200 nucleotides, about 1,000 nucleotides to about 3,000 nucleotides, about 1,000 nucleotides to about 2,600 nucleotides, about 1,000 nucleotides to about 2,400 nucleotides, about 1,000 nucleotides to about 2,200 nucleotides, about 1,000 nucleotides to about 2,000 nucleotides, about 1,000 nucleotides to about 1,800 nucleotides, about 1,000 nucleotides to about 1,600 nucleotides, about 1,000 nucleotides to about 1,400 nucleotides, about 1,000 nucleotides to about 1,200 nucleotides, about 1,200 nucleotides to about 10,000 nucleotides, about 1,200 nucleotides to about 9,500 nucleotides, about 1,200 nucleotides to about 9,000 nucleotides, about 1,200 nucleotides to about 8,500 nucleotides, about 1,200 nucleotides to about 8,000 nucleotides, about 1,200 nucleotides to about 7,800 nucleotides, about 1,200 nucleotides to about 7,600 nucleotides, about 1,200 nucleotides to about 7,400 nucleotides, about 1,200 nucleotides to about 7,200 nucleotides, about 1,200 nucleotides to about 7,000 nucleotides, about 1,200 nucleotides to about 6,800 nucleotides, about 1,200 nucleotides to about 6,600 nucleotides, about 1,200 nucleotides to about 6,400 nucleotides, about 1,200 nucleotides to about 6,200 nucleotides, about 1,200 nucleotides to about 6,000 nucleotides, about 1,200 nucleotides to about 5,800 nucleotides, about 1,200 nucleotides to about 5,600 nucleotides, about 1,200 nucleotides to about 5,400 nucleotides, about 1,200 nucleotides to about 5,000 nucleotides, about 1,200 nucleotides to about 4,800 nucleotides, about 1,200 nucleotides to about 4,600 nucleotides, about 1,200 nucleotides to about 4,400 nucleotides, about 1,200 nucleotides to about 4,200 nucleotides, about 1,200 nucleotides to about 4,000 nucleotides, about 1,200 nucleotides to about 3,800 nucleotides, about 1,200 nucleotides to about 3,600 nucleotides, about 1,200 nucleotides to about 3,400 nucleotides, about 1,200 nucleotides to about 3,200 nucleotides, about 1,200 nucleotides to about 3,000 nucleotides, about 1,200 nucleotides to about 2,800 nucleotides, about 1,200 nucleotides to about 2,600 nucleotides, about 1,200 nucleotides to about 2,400 nucleotides, about 1,200 nucleotides to about 2,200 nucleotides, about 1,200 nucleotides to about 2,000 nucleotides, about 1,200 nucleotides to about 1,800 nucleotides, about 1,200 nucleotides to about 1,600 nucleotides, about 1,200 nucleotides to about 1,400 nucleotides, about 1,400 nucleotides to about 10,000 nucleotides, about 1,400 nucleotides to about 9,500 nucleotides, about 1,400 nucleotides to about 9,000 nucleotides, about 1,400 nucleotides to about 8,500 nucleotides, about 1,400 nucleotides to about 8,000 nucleotides, about 1,400 nucleotides to about 7,800 nucleotides, about 1,400 nucleotides to about 7,600 nucleotides, about 1,400 nucleotides to about 7,400 nucleotides, about 1,400 nucleotides to about 7,200 nucleotides, about 1,400 nucleotides to about 7,000 nucleotides, about 1,400 nucleotides to about 6,800 nucleotides, about 1,400 nucleotides to about 6,600 nucleotides, about 1,400 nucleotides to about 6,400 nucleotides, about 1,400 nucleotides to about 6,200 nucleotides, about 1,400 nucleotides to about 6,000 nucleotides, about 1,400 nucleotides to about 5,800 nucleotides, about 1,400 nucleotides to about 5,600 nucleotides, about 1,400 nucleotides to about 5,400 nucleotides, about 1,400 nucleotides to about 5,200 nucleotides, about 1,400 nucleotides to about 5,000 nucleotides, about 1,400 nucleotides to about 4,800 nucleotides, about 1,400 nucleotides to about 4,600 nucleotides, about 1,400 nucleotides to about 4,400 nucleotides, about 1,400 nucleotides to about 4,200 nucleotides, about 1,400 nucleotides to about 4,000 nucleotides, about 1,400 nucleotides to about 3,800 nucleotides, about 1,400 nucleotides to about 3,600 nucleotides, about 1,400 nucleotides to about 3,400 nucleotides, about 1,400 nucleotides to about 3,200 nucleotides, about 1,400 nucleotides to about 3,000 nucleotides, about 1,400 nucleotides to about 2,600 nucleotides, about 1,400 nucleotides to about 2,400 nucleotides, about 1,400 nucleotides to about 2,200 nucleotides, about 1,400 nucleotides to about 2,000 nucleotides, about 1,400 nucleotides to about 1,800 nucleotides, about 1,400 nucleotides to about 1,600 nucleotides, about 1,600 nucleotides to about 10,000 nucleotides, about 1,600 nucleotides to about 9,500 nucleotides, about 1,600 nucleotides to about 9,000 nucleotides, about 1,600 nucleotides to about 8,500 nucleotides, about 1,600 nucleotides to about 8,000 nucleotides, about 1,600 nucleotides to about 7,800 nucleotides, about 1,600 nucleotides to about 7,600 nucleotides, about 1,600 nucleotides to about 7,400 nucleotides, about 1,600 nucleotides to about 7,200 nucleotides, about 1,600 nucleotides to about 7,000 nucleotides, about 1,600 nucleotides to about 6,800 nucleotides, about 1,600 nucleotides to about 6,400 nucleotides, about 1,600 nucleotides to about 6,200 nucleotides, about 1,600 nucleotides to about 6,000 nucleotides, about 1,600 nucleotides to about 5,800 nucleotides, about 1,600 nucleotides to about 5,600 nucleotides, about 1,600 nucleotides to about 5,400 nucleotides, about 1,600 nucleotides to about 5,200 nucleotides, about 1,600 nucleotides to about 5,000 nucleotides, about 1,600 nucleotides to about 4,800 nucleotides, about 1,600 nucleotides to about 4,600 nucleotides, about 1,600 nucleotides to about 4,400 nucleotides, about 1,600 nucleotides to about 4,200 nucleotides, about 1,600 nucleotides to about 4,000 nucleotides, about 1,600 nucleotides to about 3,800 nucleotides, about 1,600 nucleotides to about 3,600 nucleotides, about 1,600 nucleotides to about 3,400 nucleotides, about 1,600 nucleotides to about 3,200 nucleotides, about 1,600 nucleotides to about 3,000 nucleotides, about 1,600 nucleotides to about 2,800 nucleotides, about 1,600 nucleotides to about 2,600 nucleotides, about 1,600 nucleotides to about 2,400 nucleotides, about 1,600 nucleotides to about 2,200 nucleotides, about 1,600 nucleotides to about 2,000 nucleotides, about 1,600 nucleotides to about 1,800 nucleotides, about 1,800 nucleotides to about 10,000 nucleotides, about 1,800 nucleotides to about 9,500 nucleotides, about 1,800 nucleotides to about 9,000 nucleotides, about 1,800 nucleotides to about 8,500 nucleotides, about 1,800 nucleotides to about 8,000 nucleotides, about 1,800 nucleotides to about 7,800 nucleotides, about 1,800 nucleotides to about 7,600 nucleotides, about 1,800 nucleotides to about 7,400 nucleotides, about 1,800 nucleotides to about 7,200 nucleotides, about 1,800 nucleotides to about 7,000 nucleotides, about 1,800 nucleotides to about 6,800 nucleotides, about 1,800 nucleotides to about 6,600 nucleotides, about 1,800 nucleotides to about 6,400 nucleotides, about 1,800 nucleotides to about 6,200 nucleotides, about 1,800 nucleotides to about 6,000 nucleotides, about 1,800 nucleotides to about 5,800 nucleotides, about 1,800 nucleotides to about 5,600 nucleotides, about 1,800 nucleotides to about 5,400 nucleotides, about 1,800 nucleotides to about 5,200 nucleotides, about 1,800 nucleotides to about 5,000 nucleotides, about 1,800 nucleotides to about 4,800 nucleotides, about 1,800 nucleotides to about 4,600 nucleotides, about 1,800 nucleotides to about 4,400 nucleotides, about 1,800 nucleotides to about 4,200 nucleotides, about 1,800 nucleotides to about 4,000 nucleotides, about 1,800 nucleotides to about 3,800 nucleotides, about 1,800 nucleotides to about 3,600 nucleotides, about 1,800 nucleotides to about 3,400 nucleotides, about 1,800 nucleotides to about 3,200 nucleotides, about 1,800 nucleotides to about 3,000 nucleotides, about 1,800 nucleotides to about 2,800 nucleotides, about 1,800 nucleotides to about 2,600 nucleotides, about 1,800 nucleotides to about 2,400 nucleotides, about 1,800 nucleotides to about 2,200 nucleotides, about 1,800 nucleotides to about 2,000 nucleotides, about 2,000 nucleotides to about 10,000 nucleotides, about 2,000 nucleotides to about 9,500 nucleotides, about 2,000 nucleotides to about 9,000 nucleotides, about 2,000 nucleotides to about 8,500 nucleotides, about 2,000 nucleotides to about 8,000 nucleotides, about 2,000 nucleotides to about 7,800 nucleotides, about 2,000 nucleotides to about 7,600 nucleotides, about 2,000 nucleotides to about 7,400 nucleotides, about 2,000 nucleotides to about 7,200 nucleotides, about 2,000 nucleotides to about 7,000 nucleotides, about 2,000 nucleotides to about 6,800 nucleotides, about 2,000 nucleotides to about 6,600 nucleotides, about 2,000 nucleotides to about 6,400 nucleotides, about 2,000 nucleotides to about 6,200 nucleotides, about 2,000 nucleotides to about 6,000 nucleotides, about 2,000 nucleotides to about 5,800 nucleotides, about 2,000 nucleotides to about 5,600 nucleotides, about 2,000 nucleotides to about 5,400 nucleotides, about 2,000 nucleotides to about 5,200 nucleotides, about 2,000 nucleotides to about 5,000 nucleotides, about 2,000 nucleotides to about 4,800 nucleotides, about 2,000 nucleotides to about 4,600 nucleotides, about 2,000 nucleotides to about 4,400 nucleotides, about 2,000 nucleotides to about 4,200 nucleotides, about 2,000 nucleotides to about 4,000 nucleotides, about 2,000 nucleotides to about 3,800 nucleotides, about 2,000 nucleotides to about 3,600 nucleotides, about 2,000 nucleotides to about 3,400 nucleotides, about 2,000 nucleotides to about 3,200 nucleotides, about 2,000 nucleotides to about 3,000 nucleotides, about 2,000 nucleotides to about 2,800 nucleotides, about 2,000 nucleotides to about 2,600 nucleotides, about 2,000 nucleotides to about 2,400 nucleotides, about 2,000 nucleotides to about 2,200 nucleotides, about 2,200 nucleotides to about 10,000 nucleotides, about 9,500 nucleotides, about 9,000 nucleotides, about 8,500 nucleotides, about 8,000 nucleotides, about 7,800 nucleotides, about 7,600 nucleotides, about 7,400 nucleotides, about 7,200 nucleotides, about 7,000 nucleotides, about 6,800 nucleotides, about 6,600 nucleotides, about 6,400 nucleotides, about 6,200 nucleotides, about 6,000 nucleotides, about 5,800 nucleotides, about 5,600 nucleotides, about 5,400 nucleotides, about 5,200 nucleotides, about 5,000 nucleotides, about 4,800 nucleotides, about 4,600 nucleotides, about 4,400 nucleotides, about 4,200 nucleotides, about 4,000 nucleotides, about 3,800 nucleotides, about 3,600 nucleotides, about 3,400 nucleotides, about 3,200 nucleotides, about 3,000 nucleotides, about 2,800 nucleotides, about 2,600 nucleotides, about 2,400 nucleotides, about 2,400 nucleotides to about 10,000 nucleotides, about 2,400 nucleotides to about 9,500 nucleotides, about 2,400 nucleotides to about 9,000 nucleotides, about 2,400 nucleotides to about 8,500 nucleotides, about 2,400 nucleotides to about 8,000 nucleotides, about 2,400 nucleotides to about 7,800 nucleotides, about 2,400 nucleotides to about 7,600 nucleotides, about 2,400 nucleotides to about 7,400 nucleotides, about 2,400 nucleotides to about 7,200 nucleotides, about 2,400 nucleotides to about 7,000 nucleotides, about 2,400 nucleotides to about 6,800 nucleotides, about 2,400 nucleotides to about 6,600 nucleotides, about 2,400 nucleotides to about 6,400 nucleotides, about 2,400 nucleotides to about 6,200 nucleotides, about 2,400 nucleotides to about 6,000 nucleotides, about 2,400 nucleotides to about 5,800 nucleotides, about 2,400 nucleotides to about 5,600 nucleotides, about 2,400 nucleotides to about 5,400 nucleotides, about 2,400 nucleotides to about 5,200 nucleotides, about 2,400 nucleotides to about 5,000 nucleotides, about 2,400 nucleotides to about 4,800 nucleotides, about 2,400 nucleotides to about 4,600 nucleotides, about 2,400 nucleotides to about 4,400 nucleotides, about 2,400 nucleotides to about 4,200 nucleotides, about 2,400 nucleotides to about 4,000 nucleotides, about 2,400 nucleotides to about 3,800 nucleotides, about 2,400 nucleotides to about 3,600 nucleotides, about 2,400 nucleotides to about 3,400 nucleotides, about 2,400 nucleotides to about 3,200 nucleotides, about 2,400 nucleotides to about 3,000 nucleotides, about 2,400 nucleotides to about 2,800 nucleotides, about 2,400 nucleotides to about 2,600 nucleotides, about 2,600 nucleotides to about 10,000 nucleotides, about 2,600 nucleotides to about 9,500 nucleotides, about 2,600 nucleotides to about 9,000 nucleotides, about 2,600 nucleotides to about 8,500 nucleotides, about 2,600 nucleotides to about 8,000 nucleotides, about 2,600 nucleotides to about 7,800 nucleotides, about 2,600 nucleotides to about 7,600 nucleotides, about 2,600 nucleotides to about 7,400 nucleotides, about 2,600 nucleotides to about 7,200 nucleotides, about 2,600 nucleotides to about 7,000 nucleotides, about 2,600 nucleotides to about 6,800 nucleotides, about 2,600 nucleotides to about 6,600 nucleotides, about 2,600 nucleotides to about 6,400 nucleotides, about 2,600 nucleotides to about 6,200 nucleotides, about 2,600 nucleotides to about 6,000 nucleotides, about 2,600 nucleotides to about 5,800 nucleotides, about 2,600 nucleotides to about 5,600 nucleotides, about 2,600 nucleotides to about 5,400 nucleotides, about 2,600 nucleotides to about 5,200 nucleotides, about 2,600 nucleotides to about 5,000 nucleotides, about 2,600 nucleotides to about 4,800 nucleotides, about 2,600 nucleotides to about 4,600 nucleotides, about 2,600 nucleotides to about 4,400 nucleotides, about 2,600 nucleotides to about 4,200 nucleotides, about 2,600 nucleotides to about 4,000 nucleotides, about 2,600 nucleotides to about 3,800 nucleotides, about 2,600 nucleotides to about 3,600 nucleotides, about 2,600 nucleotides to about 3,400 nucleotides, about 2,600 nucleotides to about 3,200 nucleotides, about 2,600 nucleotides to about 3,000 nucleotides, about 2,600 nucleotides to about 2,800 nucleotides, about 2,800 nucleotides to about 10,000 nucleotides, about 2,800 nucleotides to about 9,500 nucleotides, about 2,800 nucleotides to about 9,000 nucleotides, about 2,800 nucleotides to about 8,500 nucleotides, about 2,800 nucleotides to about 8,000 nucleotides, about 2,800 nucleotides to about 7,800 nucleotides, about 2,800 nucleotides to about 7,600 nucleotides, about 2,800 nucleotides to about 7,400 nucleotides, about 2,800 nucleotides to about 7,200 nucleotides, about 2,800 nucleotides to about 7,000 nucleotides, about 2,800 nucleotides to about 6,800 nucleotides, about 2,800 nucleotides to about 6,600 nucleotides, about 2,800 nucleotides to about 6,400 nucleotides, about 2,800 nucleotides to about 6,200 nucleotides, about 2,800 nucleotides to about 6,000 nucleotides, about 2,800 nucleotides to about 5,800 nucleotides, about 2,800 nucleotides to about 5,600 nucleotides, about 2,800 nucleotides to about 5,400 nucleotides, about 2,800 nucleotides to about 5,200 nucleotides, about 2,800 nucleotides to about 5,000 nucleotides, about 2,800 nucleotides to about 4,800 nucleotides, about 2,800 nucleotides to about 4,600 nucleotides, about 2,800 nucleotides to about 4,400 nucleotides, about 2,800 nucleotides to about 4,200 nucleotides, about 2,800 nucleotides to about 4,000 nucleotides, about 2,800 nucleotides to about 3,800 nucleotides, about 2,800 nucleotides to about 3,600 nucleotides, about 2,800 nucleotides to about 3,400 nucleotides, about 2,800 nucleotides to about 3,200 nucleotides, about 2,800 nucleotides to about 3,000 nucleotides, about 3,000 nucleotides to about 10,000 nucleotides, about 3,000 nucleotides to about 9,500 nucleotides, about 3,000 nucleotides to about 9,000 nucleotides, about 3,000 nucleotides to about 8,500 nucleotides, about 3,000 nucleotides to about 8,000 nucleotides, about 3,000 nucleotides to about 7,800 nucleotides, about 3,000 nucleotides to about 7,600 nucleotides, about 3,000 nucleotides to about 7,400 nucleotides, about 3,000 nucleotides to about 7,200 nucleotides, about 3,000 nucleotides to about 7,000 nucleotides, about 3,000 nucleotides to about 6,800 nucleotides, about 3,000 nucleotides to about 6,600 nucleotides, about 3,000 nucleotides to about 6,400 nucleotides, about 3,000 nucleotides to about 6,200 nucleotides, about 3,000 nucleotides to about 6,000 nucleotides, about 3,000 nucleotides to about 5,800 nucleotides, about 3,000 nucleotides to about 5,600 nucleotides, about 3,000 nucleotides to about 5,400 nucleotides, about 3,000 nucleotides to about 5,200 nucleotides, about 3,000 nucleotides to about 5,000 nucleotides, about 3,000 nucleotides to about 4,800 nucleotides, about 3,000 nucleotides to about 4,600 nucleotides, about 3,000 nucleotides to about 4,400 nucleotides, about 3,000 nucleotides to about 4,200 nucleotides, about 3,000 nucleotides to about 4,000 nucleotides, about 3,000 nucleotides to about 3,800 nucleotides, about 3,000 nucleotides to about 3,600 nucleotides, about 3,000 nucleotides to about 3,400 nucleotides, about 3,000 nucleotides to about 3,200 nucleotides, about 3,200 nucleotides to about 10,000 nucleotides, about 3,200 nucleotides to about 9,500 nucleotides, about 3,200 nucleotides to about 9,000 nucleotides, about 3,200 nucleotides to about 8,500 nucleotides, about 3,200 nucleotides to about 8,000 nucleotides, about 3,200 nucleotides to about 7,800 nucleotides, about 3,200 nucleotides to about 7,600 nucleotides, about 3,200 nucleotides to about 7,400 nucleotides, about 3,200 nucleotides to about 7,200 nucleotides, about 3,200 nucleotides to about 7,000 nucleotides, about 3,200 nucleotides to about 6,800 nucleotides, about 3,200 nucleotides to about 6,600 nucleotides, about 3,200 nucleotides to about 6,400 nucleotides, about 3,200 nucleotides to about 6,200 nucleotides, about 3,200 nucleotides to about 6,000 nucleotides, about 3,200 nucleotides to about 5,800 nucleotides, about 3,200 nucleotides to about 5,600 nucleotides, about 3,200 nucleotides to about 5,400 nucleotides, about 3,200 nucleotides to about 5,200 nucleotides, about 3,200 nucleotides to about 5,000 nucleotides, about 3,200 nucleotides to about 4,800 nucleotides, about 3,200 nucleotides to about 4,600 nucleotides, about 3,200 nucleotides to about 4,400 nucleotides, about 3,200 nucleotides to about 4,200 nucleotides, about 3,200 nucleotides to about 4,000 nucleotides, about 3,200 nucleotides to about 3,800 nucleotides, about 3,200 nucleotides to about 3,600 nucleotides, about 3,200 nucleotides to about 3,400 nucleotides, about 3,400 nucleotides to about 10,000 nucleotides, about 3,400 nucleotides to about 9,500 nucleotides, about 3,400 nucleotides to about 9,000 nucleotides, about 3,400 nucleotides to about 8,500 nucleotides, about 3,400 nucleotides to about 8,000 nucleotides, about 3,400 nucleotides to about 7,800 nucleotides, about 3,400 nucleotides to about 7,600 nucleotides, about 3,400 nucleotides to about 7,400 nucleotides, about 3,400 nucleotides to about 7,200 nucleotides, about 3,400 nucleotides to about 7,000 nucleotides, about 3,400 nucleotides to about 6,800 nucleotides, about 3,400 nucleotides to about 6,600 nucleotides, about 3,400 nucleotides to about 6,400 nucleotides, about 3,400 nucleotides to about 6,200 nucleotides, about 3,400 nucleotides to about 6,000 nucleotides, about 3,400 nucleotides to about 5,800 nucleotides, about 3,400 nucleotides to about 5,600 nucleotides, about 3,400 nucleotides to about 5,400 nucleotides, about 3,400 nucleotides to about 5,200 nucleotides, about 3,400 nucleotides to about 5,000 nucleotides, about 3,400 nucleotides to about 4,800 nucleotides, about 3,400 nucleotides to about 4,600 nucleotides, about 3,400 nucleotides to about 4,400 nucleotides, about 3,400 nucleotides to about 4,200 nucleotides, about 3,400 nucleotides to about 4,000 nucleotides, about 3,400 nucleotides to about 3,800 nucleotides, about 3,400 nucleotides to about 3,600 nucleotides, about 3,600 nucleotides to about 10,000 nucleotides, about 3,600 nucleotides to about 9,500 nucleotides, about 3,600 nucleotides to about 9,000 nucleotides, about 3,600 nucleotides to about 8,500 nucleotides, about 3,600 nucleotides to about 8,000 nucleotides, about 3,600 nucleotides to about 7,800 nucleotides, about 3,600 nucleotides to about 7,600 nucleotides, about 3,600 nucleotides to about 7,400 nucleotides, about 3,600 nucleotides to about 7,200 nucleotides, about 3,600 nucleotides to about 7,000 nucleotides, about 3,600 nucleotides to about 6,800 nucleotides, about 3,600 nucleotides to about 6,600 nucleotides, about 3,600 nucleotides to about 6,400 nucleotides, about 3,600 nucleotides to about 6,200 nucleotides, about 3,600 nucleotides to about 6,000 nucleotides, about 3,600 nucleotides to about 5,800 nucleotides, about 3,600 nucleotides to about 5,600 nucleotides, about 3,600 nucleotides to about 5,400 nucleotides, about 3,600 nucleotides to about 5,200 nucleotides, about 3,600 nucleotides to about 5,000 nucleotides, about 3,600 nucleotides to about 4,800 nucleotides, about 3,600 nucleotides to about 4,600 nucleotides, about 3,600 nucleotides to about 4,400 nucleotides, about 3,600 nucleotides to about 4,200 nucleotides, about 3,600 nucleotides to about 4,000 nucleotides, about 3,600 nucleotides to about 3,800 nucleotides, about 3,800 nucleotides to about 10,000 nucleotides, about 3,800 nucleotides to about 9,500 nucleotides, about 3,800 nucleotides to about 9,000 nucleotides, about 3,800 nucleotides to about 8,500 nucleotides, about 3,800 nucleotides to about 8,000 nucleotides, about 3,800 nucleotides to about 7,800 nucleotides, about 3,800 nucleotides to about 7,600 nucleotides, about 3,800 nucleotides to about 7,400 nucleotides, about 3,800 nucleotides to about 7,200 nucleotides, about 3,800 nucleotides to about 7,000 nucleotides, about 3,800 nucleotides to about 6,800 nucleotides, about 3,800 nucleotides to about 6,600 nucleotides, about 3,800 nucleotides to about 6,400 nucleotides, about 3,800 nucleotides to about 6,200 nucleotides, about 3,800 nucleotides to about 6,000 nucleotides, about 3,800 nucleotides to about 5,800 nucleotides, about 3,800 nucleotides to about 5,600 nucleotides, about 3,800 nucleotides to about 5,400 nucleotides, about 3,800 nucleotides to about 5,200 nucleotides, about 3,800 nucleotides to about 5,000 nucleotides, about 3,800 nucleotides to about 4,800 nucleotides, about 3,800 nucleotides to about 4,600 nucleotides, about 3,800 nucleotides to about 4,200 nucleotides, about 3,800 nucleotides to about 4,000 nucleotides, about 4,000 nucleotides to about 10,000 nucleotides, about 4,000 nucleotides to about 9,500 nucleotides, about 4,000 nucleotides to about 9,000 nucleotides, about 4,000 nucleotides to about 8,500 nucleotides, about 4,000 nucleotides to about 8,000 nucleotides, about 4,000 nucleotides to about 7,800 nucleotides, about 4,000 nucleotides to about 7,600 nucleotides, about 4,000 nucleotides to about 7,400 nucleotides, about 4,000 nucleotides to about 7,200 nucleotides, about 4,000 nucleotides to about 7,000 nucleotides, about 4,000 nucleotides to about 6,800 nucleotides, about 4,000 nucleotides to about 6,600 nucleotides, about 4,000 nucleotides to about 6,400 nucleotides, about 4,000 nucleotides to about 6,200 nucleotides, about 4,000 nucleotides to about 6,000 nucleotides, about 4,000 nucleotides to about 5,800 nucleotides, about 4,000 nucleotides to about 5,600 nucleotides, about 4,000 nucleotides to about 5,400 nucleotides, about 4,000 nucleotides to about 5,200 nucleotides, about 4,000 nucleotides to about 5,000 nucleotides, about 4,000 nucleotides to about 4,800 nucleotides, about 4,000 nucleotides to about 4,600 nucleotides, about 4,000 nucleotides to about 4,400 nucleotides, about 4,000 nucleotides to about 4,200 nucleotides, about 4,200 nucleotides to about 10,000 nucleotides, about 4,200 nucleotides to about 9,500 nucleotides, about 4,200 nucleotides to about 9,000 nucleotides, about 4,200 nucleotides to about 8,500 nucleotides, about 4,200 nucleotides to about 8,000 nucleotides, about 4,200 nucleotides to about 7,800 nucleotides, about 4,200 nucleotides to about 7,600 nucleotides, about 4,200 nucleotides to about 7,400 nucleotides, about 4,200 nucleotides to about 7,200 nucleotides, about 4,200 nucleotides to about 7,000 nucleotides, about 4,200 nucleotides to about 6,800 nucleotides, about 4,200 nucleotides to about 6,600 nucleotides, about 4,200 nucleotides to about 6,400 nucleotides, about 4,200 nucleotides to about 6,200 nucleotides, about 4,200 nucleotides to about 6,000 nucleotides, about 4,200 nucleotides to about 5,800 nucleotides, about 4,200 nucleotides to about 5,600 nucleotides, about 4,200 nucleotides to about 5,400 nucleotides, about 4,200 nucleotides to about 5,200 nucleotides, about 4,200 nucleotides to about 5,000 nucleotides, about 4,200 nucleotides to about 4,800 nucleotides, about 4,200 nucleotides to about 4,600 nucleotides, about 4,200 nucleotides to about 4,400 nucleotides, about 4,400 nucleotides to about 10,000 nucleotides, about 4,400 nucleotides to about 9,500 nucleotides, about 4,400 nucleotides to about 9,000 nucleotides, about 4,400 nucleotides to about 8,500 nucleotides, about 4,400 nucleotides to about 8,000 nucleotides, about 4,400 nucleotides to about 7,800 nucleotides, about 4,400 nucleotides to about 7,600 nucleotides, about 4,400 nucleotides to about 7,400 nucleotides, about 4,400 nucleotides to about 7,200 nucleotides, about 4,400 nucleotides to about 7,000 nucleotides, about 4,400 nucleotides to about 6,800 nucleotides, about 4,400 nucleotides to about 6,600 nucleotides, about 4,400 nucleotides to about 6,400 nucleotides, about 4,400 nucleotides to about 6,200 nucleotides, about 4,400 nucleotides to about 6,000 nucleotides, about 4,400 nucleotides to about 5,800 nucleotides, about 4,400 nucleotides to about 5,600 nucleotides, about 4,400 nucleotides to about 5,400 nucleotides, about 4,400 nucleotides to about 5,200 nucleotides, about 4,400 nucleotides to about 5,000 nucleotides, about 4,400 nucleotides to about 4,800 nucleotides, about 4,400 nucleotides to about 4,600 nucleotides, about 4,600 nucleotides to about 10,000 nucleotides, about 4,600 nucleotides to about 9,500 nucleotides, about 4,600 nucleotides to about 9,000 nucleotides, about 4,600 nucleotides to about 8,500 nucleotides, about 4,600 nucleotides to about 8,000 nucleotides, about 4,600 nucleotides to about 7,800 nucleotides, about 4,600 nucleotides to about 7,600 nucleotides, about 4,600 nucleotides to about 7,400 nucleotides, about 4,600 nucleotides to about 7,200 nucleotides, about 4,600 nucleotides to about 7,000 nucleotides, about 4,600 nucleotides to about 6,800 nucleotides, about 4,600 nucleotides to about 6,600 nucleotides, about 4,600 nucleotides to about 6,400 nucleotides, about 4,600 nucleotides to about 6,200 nucleotides, about 4,600 nucleotides to about 6,000 nucleotides, about 4,600 nucleotides to about 5,800 nucleotides, about 4,600 nucleotides to about 5,600 nucleotides, about 4,600 nucleotides to about 5,400 nucleotides, about 4,600 nucleotides to about 5,200 nucleotides, about 4,600 nucleotides to about 5,000 nucleotides, about 4,600 nucleotides to about 4,800 nucleotides, about 4,800 nucleotides to about 10,000 nucleotides, about 4,800 nucleotides to about 9,500 nucleotides, about 4,800 nucleotides to about 9,000 nucleotides, about 4,800 nucleotides to about 8,500 nucleotides, about 4,800 nucleotides to about 8,000 nucleotides, about 4,800 nucleotides to about 7,800 nucleotides, about 4,800 nucleotides to about 7,600 nucleotides, about 4,800 nucleotides to about 7,400 nucleotides, about 4,800 nucleotides to about 7,200 nucleotides, about 4,800 nucleotides to about 7,000 nucleotides, about 4,800 nucleotides to about 6,800 nucleotides, about 4,800 nucleotides to about 6,600 nucleotides, about 4,800 nucleotides to about 6,400 nucleotides, about 4,800 nucleotides to about 6,200 nucleotides, about 4,800 nucleotides to about 6,000 nucleotides, about 4,800 nucleotides to about 5,800 nucleotides, about 4,800 nucleotides to about 5,600 nucleotides, about 4,800 nucleotides to about 5,400 nucleotides, about 4,800 nucleotides to about 5,200 nucleotides, about 4,800 nucleotides to about 5,000 nucleotides, about 5,000 nucleotides to about 10,000 nucleotides, about 5,000 nucleotides to about 9,500 nucleotides, about 5,000 nucleotides to about 9,000 nucleotides, about 5,000 nucleotides to about 8,500 nucleotides, about 5,000 nucleotides to about 8,000 nucleotides, about 5,000 nucleotides to about 7,800 nucleotides, about 5,000 nucleotides to about 7,600 nucleotides, about 5,000 nucleotides to about 7,400 nucleotides, about 5,000 nucleotides to about 7,200 nucleotides, about 5,000 nucleotides to about 7,000 nucleotides, about 5,000 nucleotides to about 6,800 nucleotides, about 5,000 nucleotides to about 6,600 nucleotides, about 5,000 nucleotides to about 6,400 nucleotides, about 5,000 nucleotides to about 6,200 nucleotides, about 5,000 nucleotides to about 6,000 nucleotides, about 5,000 nucleotides to about 5,800 nucleotides, about 5,000 nucleotides to about 5,600 nucleotides, about 5,000 nucleotides to about 5,400 nucleotides, about 5,000 nucleotides to about 5,200 nucleotides, about 5,200 nucleotides to about 10,000 nucleotides, about 5,200 nucleotides to about 9,500 nucleotides, about 5,200 nucleotides to about 9,000 nucleotides, about 5,200 nucleotides to about 8,500 nucleotides, about 5,200 nucleotides to about 8,000 nucleotides, about 5,200 nucleotides to about 7,800 nucleotides, about 5,200 nucleotides to about 7,600 nucleotides, about 5,200 nucleotides to about 7,400 nucleotides, about 5,200 nucleotides to about 7,200 nucleotides, about 5,200 nucleotides to about 7,000 nucleotides, about 5,200 nucleotides to about 6,800 nucleotides, about 5,200 nucleotides to about 6,600 nucleotides, about 5,200 nucleotides to about 6,400 nucleotides, about 5,200 nucleotides to about 6,200 nucleotides, about 5,200 nucleotides to about 6,000 nucleotides, about 5,200 nucleotides to about 5,800 nucleotides, about 5,200 nucleotides to about 5,600 nucleotides, about 5,200 nucleotides to about 5,400 nucleotides, about 5,400 nucleotides to about 10,000 nucleotides, about 5,400 nucleotides to about 9,500 nucleotides, about 5,400 nucleotides to about 9,000 nucleotides, about 5,400 nucleotides to about 8,500 nucleotides, about 5,400 nucleotides to about 8,000 nucleotides, about 5,400 nucleotides to about 7,800 nucleotides, about 5,400 nucleotides to about 7,600 nucleotides, about 5,400 nucleotides to about 7,400 nucleotides, about 5,400 nucleotides to about 7,200 nucleotides, about 5,400 nucleotides to about 7,000 nucleotides, about 5,400 nucleotides to about 6,800 nucleotides, about 5,400 nucleotides to about 6,600 nucleotides, about 5,400 nucleotides to about 6,400 nucleotides, about 5,400 nucleotides to about 6,200 nucleotides, about 5,400 nucleotides to about 6,000 nucleotides, about 5,400 nucleotides to about 5,800 nucleotides, about 5,400 nucleotides to about 5,600 nucleotides, about 5,600 nucleotides to about 10,000 nucleotides, about 5,600 nucleotides to about 9,500 nucleotides, about 5,600 nucleotides to about 9,000 nucleotides, about 5,600 nucleotides to about 8,500 nucleotides, about 5,600 nucleotides to about 8,000 nucleotides, about 5,600 nucleotides to about 7,800 nucleotides, about 5,600 nucleotides to about 7,600 nucleotides, about 5,600 nucleotides to about 7,400 nucleotides, about 5,600 nucleotides to about 7,200 nucleotides, about 5,600 nucleotides to about 7,000 nucleotides, about 5,600 nucleotides to about 6,800 nucleotides, about 5,600 nucleotides to about 6,600 nucleotides, about 5,600 nucleotides to about 6,400 nucleotides, about 5,600 nucleotides to about 6,200 nucleotides, about 5,600 nucleotides to about 6,000 nucleotides, about 5,600 nucleotides to about 5,800 nucleotides, about 5,800 nucleotides to about 10,000 nucleotides, about 5,800 nucleotides to about 9,500 nucleotides, about 5,800 nucleotides to about 9,000 nucleotides, about 5,800 nucleotides to about 8,500 nucleotides, about 5,800 nucleotides to about 8,000 nucleotides, about 5,800 nucleotides to about 7,800 nucleotides, about 5,800 nucleotides to about 7,600 nucleotides, about 5,800 nucleotides to about 7,400 nucleotides, about 5,800 nucleotides to about 7,200 nucleotides, about 5,800 nucleotides to about 7,000 nucleotides, about 5,800 nucleotides to about 6,800 nucleotides, about 5,800 nucleotides to abOut 6,600 nucleotides, about 5,800 nucleotides to about 6,400 nucleotides, about 5,800 nucleotides to about 6,200 nucleotides, about 5,800 nucleotides to about 6,000 nucleotides, about 6,000 nucleotides to about 10,000 nucleotides, about 6,000 nucleotides to about 9,500 nucleotides, about 6,000 nucleotides to about 9,000 nucleotides, about 6,000 nucleotides to about 8,500 nucleotides, about 6,000 nucleotides to about 8,000 nucleotides, about 6,000 nucleotides to about 7,800 nucleotides, about 6,000 nucleotides to about 7,600 nucleotides, about 6,000 nucleotides to about 7,400 nucleotides, about 6,000 nucleotides to about 7,200 nucleotides, about 6,000 nucleotides to about 7,000 nucleotides, about 6,000 nucleotides to about 6,800 nucleotides, about 6,000 nucleotides to about 6,600 nucleotides, about 6,000 nucleotides to about 6,400 nucleotides, about 6,000 nucleotides to about 6,200 nucleotides, about 6,200 nucleotides to about 10,000 nucleotides, about 6,200 nucleotides to about 9,000 nucleotides, about 6,200 nucleotides to about 8,500 nucleotides, about 6,200 nucleotides to about 8,000 nucleotides, about 6,200 nucleotides to about 7,800 nucleotides, about 6,200 nucleotides to about 7,600 nucleotides, about 6,200 nucleotides to about 7,400 nucleotides, about 6,200 nucleotides to about 7,200 nucleotides, about 6,200 nucleotides to about 7,000 nucleotides, about 6,200 nucleotides to about 6,800 nucleotides, about 6,200 nucleotides to about 6,600 nucleotides, about 6,200 nucleotides to about 6,400 nucleotides, about 6,400 nucleotides to about 10,000 nucleotides, about 6,400 nucleotides to about 9,500 nucleotides, about 6,400 nucleotides to about 9,000 nucleotides, about 6,400 nucleotides to about 8,500 nucleotides, about 6,400 nucleotides to about 8,000 nucleotides, about 6,400 nucleotides to about 7,800 nucleotides, about 6,400 nucleotides to about 7,600 nucleotides, about 6,400 nucleotides to about 7,400 nucleotides, about 6,400 nucleotides to about 7,200 nucleotides, about 6,400 nucleotides to about 7,000 nucleotides, about 6,400 nucleotides to about 6,800 nucleotides, about 6,400 nucleotides to about 6,600 nucleotides, about 6,600 nucleotides to about 10,000 nucleotides, about 6,600 nucleotides to about 9,500 nucleotides, about 6,600 nucleotides to about 9,000 nucleotides, about 6,600 nucleotides to about 8,500 nucleotides, about 6,600 nucleotides to about 8,000 nucleotides, about 6,600 nucleotides to about 7,800 nucleotides, about 6,600 nucleotides to about 7,600 nucleotides, about 6,600 nucleotides to about 7,400 nucleotides, about 6,600 nucleotides to about 7,200 nucleotides, about 6,600 nucleotides to about 7,000 nucleotides, about 6,600 nucleotides to about 6,800 nucleotides, about 6,800 nucleotides to about 10,000 nucleotides, about 6,800 nucleotides to about 9,500 nucleotides, about 6,800 nucleotides to about 9,000 nucleotides, about 6,800 nucleotides to about 8,500 nucleotides, about 6,800 nucleotides to about 8,000 nucleotides, about 6,800 nucleotides to about 7,800 nucleotides, about 6,800 nucleotides to about 7,600 nucleotides, about 6,800 nucleotides to about 7,400 nucleotides, about 6,800 nucleotides to about 7,200 nucleotides, about 6,800 nucleotides to about 7,000 nucleotides, about 7,000 nucleotides to about 10,000 nucleotides, about 7,000 nucleotides to about 9,500 nucleotides, about 7,000 nucleotides to about 9,000 nucleotides, about 7,000 nucleotides to about 8,500 nucleotides, about 7,000 nucleotides to about 8,000 nucleotides, about 7,000 nucleotides to about 7,800 nucleotides, about 7,000 nucleotides to about 7,600 nucleotides, about 7,000 nucleotides to about 7,400 nucleotides, about 7,000 nucleotides to about 7,200 nucleotides, about 7,200 nucleotides to about 10,000 nucleotides, about 7,200 nucleotides to about 9,500 nucleotides, about 7,200 nucleotides to about 9,000 nucleotides, about 7,200 nucleotides to about 8,500 nucleotides, about 7,200 nucleotides to about 8,000 nucleotides, about 7,200 nucleotides to about 7,800 nucleotides, about 7,200 nucleotides to about 7,600 nucleotides, about 7,200 nucleotides to about 7,400 nucleotides, about 7,400 nucleotides to about 10,000 nucleotides, about 7,400 nucleotides to about 9,500 nucleotides, about 7,400 nucleotides to about 9,000 nucleotides, about 7,400 nucleotides to about 8,500 nucleotides, about 7,400 nucleotides to about 8,000 nucleotides, about 7,400 nucleotides to about 7,800 nucleotides, about 7,400 nucleotides to about 7,600 nucleotides, about 7,600 nucleotides to about 10,000 nucleotides, about 7,600 nucleotides to about 9,500 nucleotides, about 7,600 nucleotides to about 9,000 nucleotides, about 7,600 nucleotides to about 8,500 nucleotides, about 7,600 nucleotides to about 8,000 nucleotides, about 7,600 nucleotides to about 7,800 nucleotides, about 7,800 nucleotides to about 10,000 nucleotides, about 7,800 nucleotides to about 9,500 nucleotides, about 7,800 nucleotides to about 9,000 nucleotides, about 7,800 nucleotides to about 8,500 nucleotides, about 7,800 nucleotides to about 8,000 nucleotides, about 8,000 nucleotides to about 10,000 nucleotides, about 8,000 nucleotides to about 9,500 nucleotides, about 8,000 nucleotides to about 9,000 nucleotides, about 8,000 nucleotides to about 8,500 nucleotides, about 8,500 nucleotides to about 10,000 nucleotides, about 8,500 nucleotides to about 9,500 nucleotides, about 8,500 nucleotides to about 9,000 nucleotides, about 9,000 nucleotides to about 10,000 nucleotides, about 9,000 nucleotides to about 9,500 nucleotides, or about 9,500 nucleotides to about 10,000 nucleotides (inclusive).

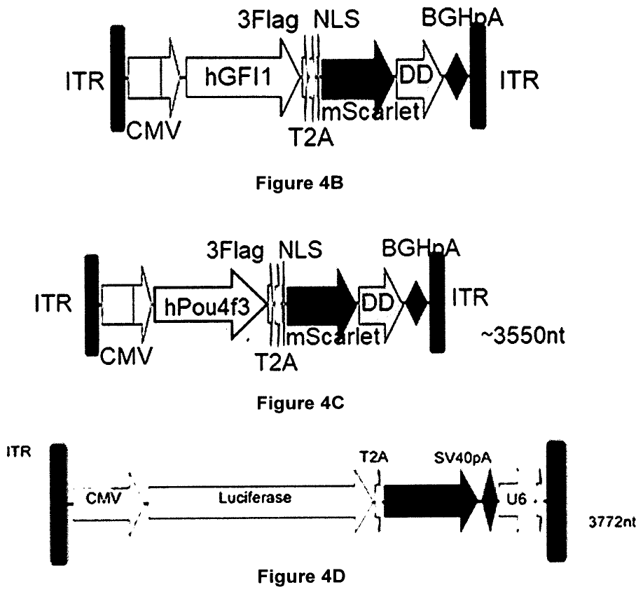

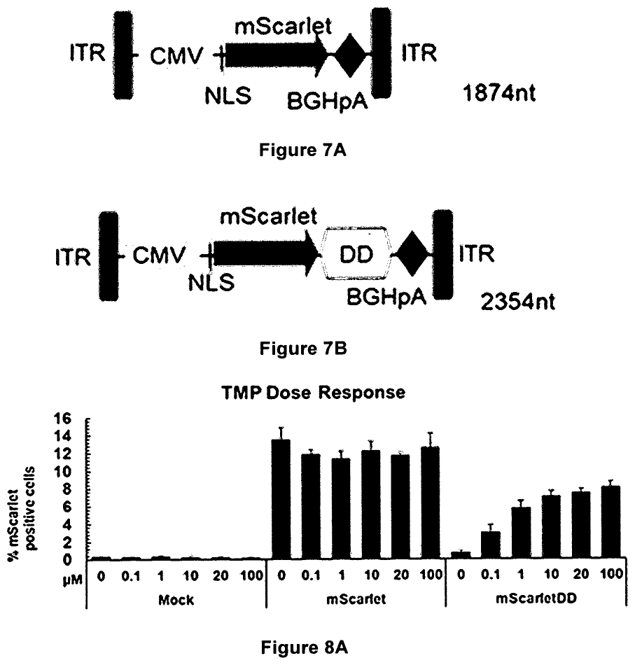

FIGS. 4A-D, FIGS. 7A-B and FIGS. 11A-B provide schematic representations of exemplary nucleic acid vectors that can be included in any of the compositions and methods described herein.

In some embodiments of any of the compositions described herein, the vector comprises or consists of pITR-CMV-mScarlet (SEQ ID NO: 50). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 50.

pITR-CMV-mScarlet
(SEQ ID NO: 50)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctctagatcccatatatggagt
tccgcgttacataacttacggtaaatggcccgcctggctgaccgcccaac
gacccccgcccattgacgtcaataatgacgtatgttcccatagtaacgcc
aatagggactttccattgacgtcaatgggtggagtatttacggtaaactg
cccacttggcagtacatcaagtgtatcatatgccaagtacgccccctatt
gacgtcaatgacggtaaatggcccgcctggcattatgcccagtacatgac
cttatgggactttcctacttggcagtacatctacgtattagtcatcgcta
ttaccatggtgatgcggttttggcagtacatcaatgggcgtggatagcgg
tttgactcacggggatttccaagtctccaccccattgacgtcaatgggag
tttgttttggcaccaaaatcaacgggactttccaaaatgtcgtaacaact
ccgccccattgacgcaaatgggcggtaggcgtgtacggtgggaggtctat
ataagcagagctcgtttagtgaaccgtcagatcgcctggagacgcatgcc
taagaagaagcggaaagtcggctccggcgtgagcaagggcgaggcagtga
tcaaggagttcatgcggttcaaggtgcacatggagggctccatgaacggc
cacgagttcgagatcgagggcgagggcgagggccgcccctacgagggcac
ccagaccgccaagctgaaggtgaccaagggtggccccctgcccttctcct
gggacatcctgtcccctcagttcatgtacggctccagggccttcatcaag
caccccgccgacatccccgactactataagcagtccttccccgagggctt
caagtgggagcgcgtgatgaacttcgaggacggcggcgccgtgaccgtga
cccaggacacctccctggaggacggcaccctgatctacaaggtgaagctc
cgcggcaccaacttccctcctgacggccccgtaatgcagaagaagacaat
gggctgggaagcgtccaccgagcggttgtaccccgaggacggcgtgctga
agggcgacattaagatggccctgcgcctgaaggacggcggccgctacctg
gcggacttcaagaccacctacaaggccaagaagcccgtgcagatgcccgg
cgcctacaacgtcgaccgcaagttggacatcacctcccacaacgaggact
acaccgtggtggaacagtacgaacgctccgagggccgccactccaccggc
ggcatggacgagctgtacaagtaagctgatcagcctcgactgtgccttct
agttgccagccatctgttgtttgcccctcccccgtgccttccttgaccct
ggaaggtgccactcccactgtcctttcctaataaaatgaggaaattgcat
cgcattgtctgagtaggtgtcattctattctggggggtggggtggggcag
gacagcaagggggaggattgggaagacaatagcaggcatgctggggatgc
ggtgggctctatggaggaacccctagtgatggagttggccactccctctc
tgcgcgctcgctcgctcactgaggccgggcgaccaaaggtcgcccgacgc
ccgggctttgcccgggcggcctcagtgagcgagcgagcgcgcagctgcct
gcagg
5′ITR cDNA sequence
(SEQ ID NO: 51)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
CMV_enhancer cDNA sequence
(SEQ ID NO: 52)
ctagatcccatatatggagttccgcgttacataacttacggtaaatggcc
cgcctggctgaccgcccaacgacccccgcccattgacgtcaataatgacg
tatgttcccatagtaacgccaatagggactttccattgacgtcaatgggt
ggagtatttacggtaaactgcccacttggcagtacatcaagtgtatcata
tgccaagtacgccccctattgacgtcaatgacggtaaatggcccgcctgg
cattatgcccagtacatgaccttatgggactttcctacttggcagtacat
ctacgtattagtcatcgctattaccatg
CMV_promoter cDNA sequence
(SEQ ID NO: 53)
gtgatgcggttttggcagtacatcaatgggcgtggatagcggtttgactc
acggggatttccaagtctccaccccattgacgtcaatgggagtttgtttt
ggcaccaaaatcaacgggactttccaaaatgtcgtaacaactccgcccca
ttgacgcaaatgggcggtaggcgtgtacggtgggaggtctatataagcag
agctcgtttagtgaaccgtcagatcgcctggagacgc
SV40-NLS cDNA sequence
(SEQ ID NO: 54)
atgcctaagaagaagcggaaagtcggctccggc
mScarlet cDNA sequence
(SEQ ID NO: 55)
gtgagcaagggcgaggcagtgatcaaggagttcatgcggttcaaggtgca
catggagggctccatgaacggccacgagttcgagatcgagggcgagggcg
agggccgcccctacgagggcacccagaccgccaagctgaaggtgaccaag
ggtggccccctgcccttctcctgggacatcctgtcccctcagttcatgta
cggctccagggccttcatcaagcaccccgccgacatccccgactactata
agcagtccttccccgagggcttcaagtgggagcgcgtgatgaacttcgag
gacggcggcgccgtgaccgtgacccaggacacctccctggaggacggcac
cctgatctacaaggtgaagctccgcggcaccaacttccctcctgacggcc
ccgtaatgcagaagaagacaatgggctgggaagcgtccaccgagcggttg
taccccgaggacggcgtgctgaagggcgacattaagatggccctgcgcct
gaaggacggcggccgctacctggcggacttcaagaccacctacaaggcca
agaagcccgtgcagatgcccggcgcctacaacgtcgaccgcaagttggac
atcacctcccacaacgaggactacaccgtggtggaacagtacgaacgctc
cgagggccgccactccaccggcggcatggacgagctgtacaagtaa
BGHpA cDNA sequence
(SEQ ID NO: 56)
gctgatcagcctcgactgtgccttctagttgccagccatctgttgtttgc
ccctcccccgtgccttccttgaccctggaaggtgccactcccactgtcct
ttcctaataaaatgaggaaattgcatcgcattgtctgagtaggtgtcatt
ctattctggggggtggggtggggcaggacagcaagggggaggattgggaa
gacaatagcaggcatgctggggatgcggtgggctctatgg
3′ ITR cDNA sequence
(SEQ ID NO: 57)
aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

In some embodiments of any of the compositions described herein, the vector comprises or consists of pITR-CMV-mScarlet-DD (SEQ ID NO: 58). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 58.

pITR-CMV-mScarlet-DD
(SEQ ID NO: 58)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctctagatcccatatatggagt
tccgcgttacataacttacggtaaatggcccgcctggctgaccgcccaac
gacccccgcccattgacgtcaataatgacgtatgttcccatagtaacgcc
aatagggactttccattgacgtcaatgggtggagtatttacggtaaactg
cccacttggcagtacatcaagtgtatcatatgccaagtacgccccctatt
gacgtcaatgacggtaaatggcccgcctggcattatgcccagtacatgac
cttatgggactttcctacttggcagtacatctacgtattagtcatcgcta
ttaccatggtgatgcggttttggcagtacatcaatgggcgtggatagcgg
tttgactcacggggatttccaagtctccaccccattgacgtcaatgggag
tttgttttggcaccaaaatcaacgggactttccaaaatgtcgtaacaact
ccgccccattgacgcaaatgggcggtaggcgtgtacggtgggaggtctat
ataagcagagctcgtttagtgaaccgtcagatcgcctggagacgcatgcc
taagaagaagcggaaagtcggctccggcgtgagcaagggcgaggcagtga
tcaaggagttcatgcggttcaaggtgcacatggagggctccatgaacggc
cacgagttcgagatcgagggcgagggcgagggccgcccctacgagggcac
ccagaccgccaagctgaaggtgaccaagggtggccccctgcccttctcct
gggacatcctgtcccctcagttcatgtacggctccagggccttcatcaag
caccccgccgacatccccgactactataagcagtccttccccgagggctt
caagtgggagcgcgtgatgaacttcgaggacggcggcgccgtgaccgtga
cccaggacacctccctggaggacggcaccctgatctacaaggtgaagctc
cgcggcaccaacttccctcctgacggccccgtaatgcagaagaagacaat
gggctgggaagcgtccaccgagcggttgtaccccgaggacggcgtgctga
agggcgacattaagatggccctgcgcctgaaggacggcggccgctacctg
gcggacttcaagaccacctacaaggccaagaagcccgtgcagatgcccgg
cgcctacaacgtcgaccgcaagttggacatcacctcccacaacgaggact
acaccgtggtggaacagtacgaacgctccgagggccgccactccaccggc
ggcatggacgagctgtacaagggtaccatcagtctgattgcggcgttagc
ggtagattacgttatcggcatggaaaacgccatgccgtggaacctgcctg
ccgatctcgcctggtttaaacgcaacaccttaaataaacccgtgattatg
ggccgccatacctgggaatcaatcggtcgtccgttgccaggacgcaaaaa
tattatcctcagcagtcaaccgagtacggacgatcgcgtaacgtgggtga
agtcggtggatgaagccatcgcggcgtgtggtgacgtaccagaaatcatg
gtgattggcggcggtcgcgttattgaacagttcttgccaaaagcgcaaaa
actgtatctgacgcatatcgacgcagaagtggaaggcgacacccatttcc
cggattacgagccggatgactgggaatcggtattcagcgaattccacgat
gctgatgcgcagaactctcacagctattgctttgagattctggagcggcg
ataagctgatcagcctcgactgtgccttctagttgccagccatctgttgt
ttgcccctcccccgtgccttccttgaccctggaaggtgccactcccactg
tcctttcctaataaaatgaggaaattgcatcgcattgtctgagtaggtgt
cattctattctggggggtggggtggggcaggacagcaagggggaggattg
ggaagacaatagcaggcatgctggggatgcggtgggctctatggaggaac
ccctagtgatggagttggccactccctctctgcgcgctcgctcgctcact
gaggccgggcgaccaaaggtcgcccgacgcccgggctttgcccgggcggc
ctcagtgagcgagcgagcgcgcagctgcctgcagg
DHFR-DD cDNA sequence
(SEQ ID NO: 59)
ggtaccatcagtctgattgcggcgttagcggtagattacgttatcggcat
ggaaaacgccatgccgtggaacctgcctgccgatctcgcctggtttaaac
gcaacaccttaaataaacccgtgattatgggccgccatacctgggaatca
atcggtcgtccgttgccaggacgcaaaaatattatcctcagcagtcaacc
gagtacggacgatcgcgtaacgtgggtgaagtcggtggatgaagccatcg
cggcgtgtggtgacgtaccagaaatcatggtgattggcggcggtcgcgtt
attgaacagttcttgccaaaagcgcaaaaactgtatctgacgcatatcga
cgcagaagtggaaggcgacacccatttcccggattacgagccggatgact
gggaatcggtattcagcgaattccacgatgctgatgcgcagaactctcac
agctattgctttgagattctggagcggcgataa

In some embodiments of any of the compositions described herein, the vector comprises or consists of pITR-CMV-hPou4f3-T2A-mScarlet-DD (SEQ ID NO: 60). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 60.

pITR-CMV-hPou4f3-T2A-mScarlet-DD 60
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctctagatcccatatatggagttccg
cgttacataacttacggtaaatggcccgcctggctgaccgcccaacgacccccgcccattgacgtcaataatgacgt
atgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcatta
tgcccagtacatgaccttatgggactttcctacttggcagtacatctacgtattagtcatcgctattaccatggtga
tgcggttttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgca
aatgggcggtaggcgtgtacggtgggaggtctatataagcagagctcgtttagtgaaccgtcagatcgcctggagac
gcatgatggccatgaactccaagcagcctttcggcatgcacccggtgctgcaagaacccaaattctccagtctgcac
tctggctccgaggccatgcgccgagtctgtctcccagccccgcagctgcagggtaatatatttggaagctttgatga
gagcctgctggcacgcgccgaagctctggcggcggtggatatcgtctcccacggcaagaaccatccgttcaagcccg
acgccacctaccataccatgagcagcgtgccctgcacgtccacttcgtccaccgtgcccatctcccacccagctgcg
ctcacctcacaccctcaccacgccgtgcaccagggcctcgaaggcgacctgctggagcacatctcgcccacgctgag
tgtgagcggcctgggcgctccggaacactcggtgatgcccgcacagatccatccacaccacctgggcgccatgggcc
acctgcaccaggccatgggcatgagtcacccgcacaccgtggcccctcatagcgccatgcctgcatgcctcagcgac
gtggagtcagacccgcgcgagctggaagccttcgccgagcgcttcaagcagcggcgcatcaagctgggggtgaccca
ggcggacgtgggcgcggctctggctaatctcaagatccccggcgtgggctcgctgagccaaagcaccatctgcaggt
tcgagtctctcactctctcgcacaacaacatgatcgctctcaagccggtgctccaggcctggttggaggaggccgag
gccgcctaccgagagaagaacagcaagccagagctcttcaacggcagcgaacggaagcgcaaacgcacgtccatcgc
ggcgccggagaagcgttcactcgaggcctatttcgctatccagccacgtccttcatctgagaagatcgcggccatcg
ctgagaaactggaccttaaaaagaacgtggtgagagtctggttctgcaaccagagacagaaacagaaacgaatgaag
tattcggctgtccacgttaacgattacaaggatgacgacgataaggactataaggacgatgatgacaaggactacaa
agatgatgacgataaaggatccggcgagggcagaggaagtctgctaacatgcggtgacgtcgaggagaatcctggcc
caatgcctaagaagaagcggaaagtcggctccggcgtgagcaagggcgaggcagtgatcaaggagttcatgcggttc
aaggtgcacatggagggctccatgaacggccacgagttcgagatcgagggcgagggcgagggccgcccctacgaggg
cacccagaccgccaagctgaaggtgaccaagggtggccccctgcccttctcctgggacatcctgtcccctcagttca
tgtacggctccagggccttcatcaagcaccccgccgacatccccgactactataagcagtccttccccgagggcttc
aagtgggagcgcgtgatgaacttcgaggacggcggcgccgtgaccgtgacccaggacacctccctggaggacggcac
cctgatctacaaggtgaagctccgcggcaccaacttccctcctgacggccccgtaatgcagaagaagacaatgggct
gggaagcgtccaccgagcggttgtaccccgaggacggcgtgctgaagggcgacattaagatggccctgcgcctgaag
gacggcggccgctacctggcggacttcaagaccacctacaaggccaagaagcccgtgcagatgcccggcgcctacaa
cgtcgaccgcaagttggacatcacctcccacaacgaggactacaccgtggtggaacagtacgaacgctccgagggcc
gccactccaccggcggcatggacgagctgtacaagggtaccatcagtctgattgcggcgttagcggtagattacgtt
atcggcatggaaaacgccatgccgtggaacctgcctgccgatctcgcctggtttaaacgcaacaccttaaataaacc
cgtgattatgggccgccatacctgggaatcaatcggtcgtccgttgccaggacgcaaaaatattatcctcagcagtc
aaccgagtacggacgatcgcgtaacgtgggtgaagtcggtggatgaagccatcgcggcgtgtggtgacgtaccagaa
atcatggtgattggcggcggtcgcgttattgaacagttcttgccaaaagcgcaaaaactgtatctgacgcatatcga
cgcagaagtggaaggcgacacccatttcccggattacgagccggatgactgggaatcggtattcagcgaattccacg
atgctgatgcgcagaactctcacagctattgctttgagattctggagcggcgataagctgatcagcctcgactgtgc
cttctagttgccagccatctgttgtttgcccctcccccgtgccttccttgaccctggaaggtgccactcccactgtc
ctttcctaataaaatgaggaaattgcatcgcattgtctgagtaggtgtcattctattctggggggtggggtggggca
ggacagcaagggggaggattgggaagacaatagcaggcatgctggggatgcggtgggctctatggaggaacccctag
tgatggagttggccactccctctctgcgcgctcgctcgctcactgaggccgggcgaccaaaggtcgcccgacgcccg
ggctttgcccgggcggcctcagtgagcgagcgagcgcgcagctgcctgcagg
hPou4f3 cDNA sequence
(SEQ ID NO: 61)
atgatggccatgaactccaagcagcctttcggcatgcacccggtgctgcaagaacccaaattctccagtctgcactc
tggctccgaggccatgcgccgagtctgtctcccagccccgcagctgcagggtaatatatttggaagctttgatgaga
gcctgctggcacgcgccgaagctctggcggcggtggatatcgtctcccacggcaagaaccatccgttcaagcccgac
gccacctaccataccatgagcagcgtgccctgcacgtccacttcgtccaccgtgcccatctcccacccagctgcgct
cacctcacaccctcaccacgccgtgcaccagggcctcgaaggcgacctgctggagcacatctcgcccacgctgagtg
tgagcggcctgggcgctccggaacactcggtgatgcccgcacagatccatccacaccacctgggcgccatgggccac
ctgcaccaggccatgggcatgagtcacccgcacaccgtggcccctcatagcgccatgcctgcatgcctcagcgacgt
ggagtcagacccgcgcgagctggaagccttcgccgagcgcttcaagcagcggcgcatcaagctgggggtgacccagg
cggacgtgggcgcggctctggctaatctcaagatccccggcgtgggctcgctgagccaaagcaccatctgcaggttc
gagtctctcactctctcgcacaacaacatgatcgctctcaagccggtgctccaggcctggttggaggaggccgaggc
cgcctaccgagagaagaacagcaagccagagctcttcaacggcagcgaacggaagcgcaaacgcacgtccatcgcgg
cgccggagaagcgttcactcgaggcctatttcgctatccagccacgtccttcatctgagaagatcgcggccatcgct
gagaaactggaccttaaaaagaacgtggtgagagtctggttctgcaaccagagacagaaacagaaacgaatgaagta
ttcggctgtccacgttaac
3x FLAG cDNA sequence
(SEQ ID NO: 62)
gattacaaggatgacgacgataaggactataaggacgatgatgacaaggactacaaagatgatgacgataaaggatc
cggc
T2A cDNA sequence
(SEQ ID NO: 63)
gagggcagaggaagtctgctaacatgcggtgacgtcgaggagaatcctggccca
T2A cDNA sequence
(SEQ ID NO: 89)
GCGAGGGCAGAGGAAGTCTGCTAACATGCGGTGACGTCGAGGAGAATCCTGGCCCA

In some embodiments of any of the compositions described herein, the vector comprises or consists of pITR-CMV-hGFI1-T2A-mScarlet-DD (SEQ ID NO: 64). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 64.

pITR-CMV-hGFI1-T2A-mScarlet-DD
(SEQ ID NO: 64)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctctagatcccatatatggagttccg
cgttacataacttacggtaaatggcccgcctggctgaccgcccaacgacccccgcccattgacgtcaataatgacgt
atgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcatta
tgcccagtacatgaccttatgggactttcctacttggcagtacatctacgtattagtcatcgctattaccatggtga
tgcggttttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgca
aatgggcggtaggcgtgtacggtgggaggtctatataagcagagctcgtttagtgaaccgtcagatcgcctggagac
gcatgccgcgctcatttctcgtcaaaagcaagaaggctcacagctaccaccagccgcgctccccaggaccagactat
tccctccgtttagagaatgtaccggcgcctagccgagcagacagcacttcaaatgcaggcggggcgaaggcggagcc
ccgggaccgtttgtcccccgaatcgcagctgaccgaagccccagacagagcctccgcatccccagacagctgcgaag
gcagcgtctgcgaacggagctcggagtttgaggacttctggaggcccccgtcaccctccgcgtctccagcctcggag
aagtcaatgtgcccatcgctggacgaagcccagcccttccccctgcctttcaaaccgtactcatggagcggcctggc
gggttctgacctgcggcacctggtgcagagctaccgaccgtgtggggccctggagcgtggcgctggcctgggcctct
tctgcgaacccgccccggagcctggccacccggccgcgctgtacggcccgaagcgggctgccggcggcgcgggggcc
ggggcgccagggagctgcagcgcaggggccggtgccaccgctggccctggcctagggctctacggcgacttcgggtc
tgcggcagccgggctgtatgagaggcccacggcagcggcgggcttgctgtaccccgagcgtggccacgggctgcacg
cagacaagggcgctggcgtcaaggtggagtcggagctgctgtgcacccgcctgctgctgggcggcggctcctacaag
tgcatcaagtgcagcaaggtgttctccacgccgcacgggctcgaggtgcacgtgcgcaggtcccacagcggcaccag
accctttgcctgcgagatgtgcggcaagaccttcgggcacgcggtgagcctggagcagcacaaagccgtgcactcgc
aggaacggagctttgactgtaagatctgtgggaagagcttcaagaggtcatccacactgtccacacacctgcttatc
cactcagacactcggccctacccctgtcagtactgtggcaagaggttccaccagaagtcagacatgaagaaacacac
tttcatccacactggtgagaagcctcacaagtgccaggtgtgcggcaaggcattcagccagagctccaacctcatca
cccacagccgcaaacacacaggcttcaagcccttcggctgcgacctctgtgggaagggtttccagaggaaggtggac
ctccgaaggcaccgggagacgcagcatgggctcaaagttaacgattacaaggatgacgacgataaggactataagga
cgatgatgacaaggactacaaagatgatgacgataaaggatccggcgagggcagaggaagtctgctaacatgcggtg
acgtcgaggagaatcctggcccaatgcctaagaagaagcggaaagtcggctccggcgtgagcaagggcgaggcagtg
atcaaggagttcatgcggttcaaggtgcacatggagggctccatgaacggccacgagttcgagatcgagggcgaggg
cgagggccgcccctacgagggcacccagaccgccaagctgaaggtgaccaagggtggccccctgcccttctcctggg
acatcctgtcccctcagttcatgtacggctccagggccttcatcaagcaccccgccgacatccccgactactataag
cagtccttccccgagggcttcaagtgggagcgcgtgatgaacttcgaggacggcggcgccgtgaccgtgacccagga
cacctccctggaggacggcaccctgatctacaaggtgaagctccgcggcaccaacttccctcctgacggccccgtaa
tgcagaagaagacaatgggctgggaagcgtccaccgagcggttgtaccccgaggacggcgtgctgaagggcgacatt
aagatggccctgcgcctgaaggacggcggccgctacctggcggacttcaagaccacctacaaggccaagaagcccgt
gcagatgcccggcgcctacaacgtcgaccgcaagttggacatcacctcccacaacgaggactacaccgtggtggaac
agtacgaacgctccgagggccgccactccaccggcggcatggacgagctgtacaagggtaccatcagtctgattgcg
gcgttagcggtagattacgttatcggcatggaaaacgccatgccgtggaacctgcctgccgatctcgcctggtttaa
acgcaacaccttaaataaacccgtgattatgggccgccatacctgggaatcaatcggtcgtccgttgccaggacgca
aaaatattatcctcagcagtcaaccgagtacggacgatcgcgtaacgtgggtgaagtcggtggatgaagccatcgcg
gcgtgtggtgacgtaccagaaatcatggtgattggcggcggtcgcgttattgaacagttcttgccaaaagcgcaaaa
actgtatctgacgcatatcgacgcagaagtggaaggcgacacccatttcccggattacgagccggatgactgggaat
cggtattcagcgaattccacgatgctgatgcgcagaactctcacagctattgctttgagattctggagcggcgataa
gctgatcagcctcgactgtgccttctagttgccagccatctgttgtttgcccctcccccgtgccttccttgaccctg
gaaggtgccactcccactgtcctttcctaataaaatgaggaaattgcatcgcattgtctgagtaggtgtcattctat
tctggggggtggggtggggcaggacagcaagggggaggattgggaagacaatagcaggcatgctggggatgcggtgg
gctctatggaggaacccctagtgatggagttggccactccctctctgcgcgctcgctcgctcactgaggccgggcga
ccaaaggtcgcccgacgcccgggctttgcccgggcggcctcagtgagcgagcgagcgcgcagctgcctgcagg
hGFI1 cDNA sequence
(SEQ ID NO: 65)
ATGCCGCGCTCATTTCTCGTCAAAAGCAAGAAGGCTCACAGCTACCACCAGCCGCGCTCCCCAGGACCAGACTATTC
CCTCCGTTTAGAGAATGTACCGGCGCCTAGCCGAGCAGACAGCACTTCAAATGCAGGCGGGGCGAAGGCGGAGCCCC
GGGACCGTTTGTCCCCCGAATCGCAGCTGACCGAAGCCCCAGACAGAGCCTCCGCATCCCCAGACAGCTGCGAAGGC
AGCGTCTGCGAACGGAGCTCGGAGTTTGAGGACTTCTGGAGGCCCCCGTCACCCTCCGCGTCTCCAGCCTCGGAGAA
GTCAATGTGCCCATCGCTGGACGAAGCCCAGCCCTTCCCCCTGCCTTTCAAACCGTACTCATGGAGCGGCCTGGCGG
GTTCTGACCTGCGGCACCTGGTGCAGAGCTACCGACCGTGTGGGGCCCTGGAGCGTGGCGCTGGCCTGGGCCTCTTC
TGCGAACCCGCCCCGGAGCCTGGCCACCCGGCCGCGCTGTACGGCCCGAAGCGGGCTGCCGGCGGCGCGGGGGCCGG
GGCGCCAGGGAGCTGCAGCGCAGGGGCCGGTGCCACCGCTGGCCCTGGCCTAGGGCTCTACGGCGACTTCGGGTCTG
CGGCAGCCGGGCTGTATGAGAGGCCCACGGCAGCGGCGGGCTTGCTGTACCCCGAGCGTGGCCACGGGCTGCACGCA
GACAAGGGCGCTGGCGTCAAGGTGGAGTCGGAGCTGCTGTGCACCCGCCTGCTGCTGGGCGGCGGCTCCTACAAGTG
CATCAAGTGCAGCAAGGTGTTCTCCACGCCGCACGGGCTCGAGGTGCACGTGCGCAGGTCCCACAGCGGCACCAGAC
CCTTTGCCTGCGAGATGTGCGGCAAGACCTTCGGGCACGCGGTGAGCCTGGAGCAGCACAAAGCCGTGCACTCGCAG
GAACGGAGCTTTGACTGTAAGATCTGTGGGAAGAGCTTCAAGAGGTCATCCACACTGTCCACACACCTGCTTATCCA
CTCAGACACTCGGCCCTACCCCTGTCAGTACTGTGGCAAGAGGTTCCACCAGAAGTCAGACATGAAGAAACACACTT
TCATCCACACTGGTGAGAAGCCTCACAAGTGCCAGGTGTGCGGCAAGGCATTCAGCCAGAGCTCCAACCTCATCACC
CACAGCCGCAAACACACAGGCTTCAAGCCCTTCGGCTGCGACCTCTGTGGGAAGGGTTTCCAGAGGAAGGTGGACCT
CCGAAGGCACCGGGAGACGCAGCATGGGCTCAAAGTTAAC

In some embodiments of any of the compositions described herein, the vector comprises or consists of pITR-CMV-hATOH1-T2A-mScarlet-DD (SEQ ID NO: 66). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 66.

pITR-CMV-hATOH1-T2A-mScarlet-DD
(SEQ ID NO: 66)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctctagatcccatatatggagttccg
cgttacataacttacggtaaatggcccgcctggctgaccgcccaacgacccccgcccattgacgtcaataatgacgt
atgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcatta
tgcccagtacatgaccttatgggactttcctacttggcagtacatctacgtattagtcatcgctattaccatggtga
tgcggttttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgca
aatgggcggtaggcgtgtacggtgggaggtctatataagcagagctcgtttagtgaaccgtcagatcgcctggagac
gcatgtcccgcctgctgcatgcagaagagtgggctgaagtgaaggagttgggagaccaccatcgccagccccagccg
catcatctcccgcaaccgccgccgccgccgcagccacctgcaactttgcaggcgagagagcatcccgtctacccgcc
tgagctgtccctcctggacagcaccgacccacgcgcctggctggctcccactttgcagggcatctgcacggcacgcg
ccgcccagtatttgctacattccccggagctgggtgcctcagaggccgctgcgccccgggacgaggtggacggccgg
ggggagctggtaaggaggagcagcggcggtgccagcagcagcaagagccccgggccggtgaaagtgcgggaacagct
gtgcaagctgaaaggcggggtggtggtagacgagctgggctgcagccgccaacgggccccttccagcaaacaggtga
atggggtgcagaagcagagacggctagcagccaacgccagggagcggcgcaggatgcatgggctgaaccacgccttc
gaccagctgcgcaatgttatcccgtcgttcaacaacgacaagaagctgtccaaatatgagaccctgcagatggccca
aatctacatcaacgccttgtccgagctgctacaaacgcccagcggaggggaacagccaccgccgcctccagcctcct
gcaaaagcgaccaccaccaccttcgcaccgcggcctcctatgaagggggcgcgggcaacgcgaccgcagctggggct
cagcaggcttccggagggagccagcggccgaccccgcccgggagttgccggactcgcttctcagccccagcttctgc
gggagggtactcggtgcagctggacgctctgcacttctcgactttcgaggacagcgccctgacagcgatgatggcgc
aaaagaatttgtctccttctctccccgggagcatcttgcagccagtgcaggaggaaaacagcaaaacttcgcctcgg
tcccacagaagcgacggggaattttccccccattcccattacagtgactcggatgaggcaagtgttaacgattacaa
ggatgacgacgataaggactataaggacgatgatgacaaggactacaaagatgatgacgataaaggatccggcgagg
gcagaggaagtctgctaacatgcggtgacgtcgaggagaatcctggcccaatgcctaagaagaagcggaaagtcggc
tccggcgtgagcaagggcgaggcagtgatcaaggagttcatgcggttcaaggtgcacatggagggctccatgaacgg
ccacgagttcgagatcgagggcgagggcgagggccgcccctacgagggcacccagaccgccaagctgaaggtgacca
agggtggccccctgcccttctcctgggacatcctgtcccctcagttcatgtacggctccagggccttcatcaagcac
cccgccgacatccccgactactataagcagtccttccccgagggcttcaagtgggagcgcgtgatgaacttcgagga
cggcggcgccgtgaccgtgacccaggacacctccctggaggacggcaccctgatctacaaggtgaagctccgcggca
ccaacttccctcctgacggccccgtaatgcagaagaagacaatgggctgggaagcgtccaccgagcggttgtacccc
gaggacggcgtgctgaagggcgacattaagatggccctgcgcctgaaggacggcggccgctacctggcggacttcaa
gaccacctacaaggccaagaagcccgtgcagatgcccggcgcctacaacgtcgaccgcaagttggacatcacctccc
acaacgaggactacaccgtggtggaacagtacgaacgctccgagggccgccactccaccggcggcatggacgagctg
tacaagggtaccatcagtctgattgcggcgttagcggtagattacgttatcggcatggaaaacgccatgccgtggaa
cctgcctgccgatctcgcctggtttaaacgcaacaccttaaataaacccgtgattatgggccgccatacctgggaat
caatcggtcgtccgttgccaggacgcaaaaatattatcctcagcagtcaaccgagtacggacgatcgcgtaacgtgg
gtgaagtcggtggatgaagccatcgcggcgtgtggtgacgtaccagaaatcatggtgattggcggcggtcgcgttat
tgaacagttcttgccaaaagcgcaaaaactgtatctgacgcatatcgacgcagaagtggaaggcgacacccatttcc
cggattacgagccggatgactgggaatcggtattcagcgaattccacgatgctgatgcgcagaactctcacagctat
tgctttgagattctggagcggcgataagctgatcagcctcgactgtgccttctagttgccagccatctgttgtttgc
ccctcccccgtgccttccttgaccctggaaggtgccactcccactgtcctttcctaataaaatgaggaaattgcatc
gcattgtctgagtaggtgtcattctattctggggggtggggtggggcaggacagcaagggggaggattgggaagaca
atagcaggcatgctggggatgcggtgggctctatggaggaacccctagtgatggagttggccactccctctctgcgc
gctcgctcgctcactgaggccgggcgaccaaaggtcgcccgacgcccgggctttgcccgggcggcctcagtgagcga
gcgagcgcgcagctgcctgcagg
hATOH1 cDNA sequence
(SEQ ID NO: 67)
atgtcccgcctgctgcatgcagaagagtgggctgaagtgaaggagttgggagaccaccatcgccagccccagccgca
tcatctcccgcaaccgccgccgccgccgcagccacctgcaactttgcaggcgagagagcatcccgtctacccgcctg
agctgtccctcctggacagcaccgacccacgcgcctggctggctcccactttgcagggcatctgcacggcacgcgcc
gcccagtatttgctacattccccggagctgggtgcctcagaggccgctgcgccccgggacgaggtggacggccgggg
ggagctggtaaggaggagcagcggcggtgccagcagcagcaagagccccgggccggtgaaagtgcgggaacagctgt
gcaagctgaaaggcggggtggtggtagacgagctgggctgcagccgccaacgggccccttccagcaaacaggtgaat
ggggtgcagaagcagagacggctagcagccaacgccagggagcggcgcaggatgcatgggctgaaccacgccttcga
ccagctgcgcaatgttatcccgtcgttcaacaacgacaagaagctgtccaaatatgagaccctgcagatggcccaaa
tctacatcaacgccttgtccgagctgctacaaacgcccagcggaggggaacagccaccgccgcctccagcctcctgc
aaaagcgaccaccaccaccttcgcaccgcggcctcctatgaagggggcgcgggcaacgcgaccgcagctggggctca
gcaggcttccggagggagccagcggccgaccccgcccgggagttgccggactcgcttctcagccccagcttctgcgg
gagggtactcggtgcagctggacgctctgcacttctcgactttcgaggacagcgccctgacagcgatgatggcgcaa
aagaatttgtctccttctctccccgggagcatcttgcagccagtgcaggaggaaaacagcaaaacttcgcctcggtc
ccacagaagcgacggggaattttccccccattcccattacagtgactcggatgaggcaagtgttaac

In some embodiments of any of the compositions described herein, the vector comprises or consists of pITR-CMV-Luc-T2A-mScarlet-U6-Hes1-S3 (SEQ ID NO: 68). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 68.

pITR-CMV-Luc2-T2A-mScarlet-U6-Hes1-S3
(SEQ ID NO: 68)
cctgcaggcagctgcgcgctcgctcgctcactgaggccgcccgggcgtcg
ggcgacctttggtcgcceggcctcagtgagcgagcgagcgcgcagagagg
gagtggccaactccatcactaggggttcctctagatcccatatatggagt
tccgcgttacataacttacggtaaatggcccgcctggctgaccgcccaac
gacccccgcccattgacgtcaataatgacgtatgttcccatagtaacgcc
aatagggactttccattgacgtcaatgggtggagtatttacggtaaactg
cccacttggcagtacatcaagtgtatcatatgccaagtacgccccctatt
gacgtcaatgacggtaaatggcccgcctggcattatgcccagtacatgac
cttatgggactttcctacttggcagtacatctacgtattagtcatcgcta
ttaccatggtgatgcggttttggcagtacatcaatgggcgtggatagcgg
tttgactcacggggatttccaagtctccaccccattgacgtcaatgggag
tttgttttggcaccaaaatcaacgggactttccaaaatgtcgtaacaact
ccgccccattgacgcaaatgggcggtaggcgtgtacggtgggaggtctat
ataagcagagctcgtttagtgaaccgtcagatcgcctggagacgcatgga
agatgccaaaaacattaagaagggcccagcgccattctacccactcgaag
acgggaccgccggcgagcagctgcacaaagccatgaagcgctacgccctg
gtgcccggcaccatcgcctttaccgacgcacatatcgaggtggacattac
ctacgccgagtacttcgagatgagcgttcggctggcagaagctatgaagc
gctatgggctgaatacaaaccatcggatcgtggtgtgcagcgagaatagc
ttgcagttcttcatgcccgtgttgggtgccctgttcatcggtgtggctgt
ggccccagctaacgacatctacaacgagcgcgagctgctgaacagcatgg
gcatcagccagcccaccgtcgtattcgtgagcaagaaagggctgcaaaag
atcctcaacgtgcaaaagaagctaccgatcatacaaaagatcatcatcat
ggatagcaagaccgactaccagggcttccaaagcatgtacaccttcgtga
cttcccatttgccacccggcttcaacgagtacgacttcgtgcccgagagc
ttcgaccgggacaaaaccatcgccctgatcatgaacagtagtggcagtac
cggattgcccaagggcgtagccctaccgcaccgcaccgcttgtgtccgat
tcagtcatgcccgcgaccccatcttcggcaaccagatcatccccgacacc
gctatcctcagcgtggtgccatttcaccacggcttcggcatgttcaccac
gctgggctacttgatctgcggctttcgggtcgtgctcatgtaccgcttcg
aggaggagctattcttgcgcagcttgcaagactataagattcaatctgcc
ctgctggtgcccacactatttagcttcttcgctaagagcactctcatcga
caagtacgacctaagcaacttgcacgagatcgccagcggcggggcgccgc
tcagcaaggaggtaggtgaggccgtggccaaacgcttccacctaccaggc
atccgccagggctacggcctgacagaaacaaccagcgccattctgatcac
ccccgaaggggacgacaagcctggcgcagtaggcaaggtggtgcccttct
tcgaggctaaggtggtggacttggacacaggtaagacactgggtgtgaac
cagcgcggcgagctgtgcgtccgtggccccatgatcatgagcggctacgt
taacaaccccgaggctacaaacgctctcatcgacaaggacggctggctgc
acagcggcgacatcgcctactgggacgaggacgagcacttcttcatcgtg
gaccggctgaagagcctgatcaaatacaagggctaccaggtagccccagc
cgaactggagagcatcctgctgcaacaccccaacatcttcgacgccgggg
tcgccggcctgcccgacgacgatgccggcgagctgcccgccgcagtcgtc
gtgctggaacacggtaaaaccatgaccgagaaggagatcgtggactatgt
ggccagccaggttacaaccgccaagaagctgcgcggtggtgttgtgttcg
tggacgaggtgcctaaaggactgaccggcaagttggacgcccgcaagatc
cgcgagattctcattaaggccaagaagggcggcaagatcgccgtgggctc
cggagagggcagaggaagtctgctaacatgcggtgacgtcgaggagaatc
ctggcccaatggtgagcaagggcgaggcagtgatcaaggagttcatgcgg
ttcaaggtgcacatggagggctccatgaacggccacgagttcgagatcga
gggcgagggcgagggccgcccctacgagggcacccagaccgccaagctga
aggtgaccaagggtggccccctgcccttctcctgggacatcctgtcccct
cagttcatgtacggctccagggccttcatcaagcaccccgccgacatccc
cgactactataagcagtccttccccgagggcttcaagtgggagcgcgtga
tgaacttcgaggacggcggcgccgtgaccgtgacccaggacacctccctg
gaggacggcaccctgatctacaaggtgaagctccgcggcaccaacttccc
tcctgacggccccgtaatgcagaagaagacaatgggctgggaagcgtcca
ccgagcggttgtaccccgaggacggcgtgctgaagggcgacattaagatg
gccctgcgcctgaaggacggcggccgctacctggcggacttcaagaccac
ctacaaggccaagaagcccgtgcagatgcccggcgcctacaacgtcgacc
gcaagttggacatcacctcccacaacgaggactacaccgtggtggaacag
tacgaacgctccgagggccgccactccaccggcggcatggacgagctgta
caagtaagctgatcagcctcgataagatacattgatgagtttggacaaac
cacaactagaatgcagtgaaaaaaatgctttatttgtgaaatttgtgatg
ctattgctttatttgtaaccattataagctgcaataaacaagttaaggtc
gggcaggaagagggcctatttcccatgattccttcatatttgcatatacg
atacaaggctgttagagagataattagaattaatttgactgtaaacacaa
agatattagtacaaaatacgtgacgtagaaagtaataatttcttgggtag
tttgcagttttaaaattatgttttaaaatggactatcatatgcttaccgt
aacttgaaagtatttcgatttcttggctttatatatcttgtggaaaggac
gaaacaccgaaagtcatcaaagcctatcgaaataggctttgatgactttc
ttttttaggaacccctagtgatggagttggccactccctctctgcgcgct
cgctcgctcactgaggccgggcgaccaaaggtcgcccgacgcccgggctt
tgcccgggcggcctcagtgagcgagcgagcgcgcagctgc

In some embodiments of any of the compositions described herein, the vector comprises or consists of pITR-CMV-Luc-T2A-GFP-U6-Hes1-S5 (SEQ ID NO: 78). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 78.

pITR-CMV-Luc2-T2A-GFP-U6-Hes1 -S5
(SEQ ID NO: 78)
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTCTAGATCCCATATATGGAGT
TCCGCGTTACATAACTTACGGTAAATGGCCCGCCTGGCTGACCGCCCAAC
GACCCCCGCCCATTGACGTCAATAATGACGTATGTTCCCATAGTAACGCC
AATAGGGACTTTCCATTGACGTCAATGGGTGGAGTATTTACGGTAAACTG
CCCACTTGGCAGTACATCAAGTGTATCATATGCCAAGTACGCCCCCTATT
GACGTCAATGACGGTAAATGGCCCGCCTGGCATTATGCCCAGTACATGAC
CTTATGGGACTTTCCTACTTGGCAGTACATCTACGTATTAGTCATCGCTA
TTACCATGGTGATGCGGTTTTGGCAGTACATCAATGGGCGTGGATAGCGG
TTTGACTCACGGGGATTTCCAAGTCTCCACCCCATTGACGTCAATGGGAG
TTTGTTTTGGCACCAAAATCAACGGGACTTTCCAAAATGTCGTAACAACT
CCGCCCCATTGACGCAAATGGGCGGTAGGCGTGTACGGTGGGAGGTCTAT
ATAAGCAGAGCTCGTTTAGTGAACCGTCAGATCGCCTGGAGACGCATGGA
AGATGCCAAAAACATTAAGAAGGGCCCAGCGCCATTCTACCCACTCGAAG
ACGGGACCGCCGGCGAGCAGCTGCACAAAGCCATGAAGCGCTACGCCCTG
GTGCCCGGCACCATCGCCTTTACCGACGCACATATCGAGGTGGACATTAC
CTACGCCGAGTACTTCGAGATGAGCGTTCGGCTGGCAGAAGCTATGGGCT
GAATGGGCTGTAATACAAACCATCGGATCGTGGTGTGCAGCGAGAATAGC
TTGCAGTTCTTCATGCCCGTGTTGGGTGCCCTGTTCATCGGTGTGGCTGT
GGCCCCAGCTAACGACATCTACAACGAGCGCGAGCTGCTGAACAGCATGG
GCATCAGCCAGCCCACCGTCGTATTCGTGAGCAAGAAAGGGCTGCAAAAG
ATCCTCAACGTGCAAAAGAAGCTACCGATCATACAAAAGATCATCATCAT
GGATAGCAAGACCGACTACCAGGGCTTCCAAAGCATGTACACCTTCGTGA
CTTCCCATTTGCCACCCGGCTTCAACGAGTACGACTTCGTGCCCGAGAGC
TTCGACCGGGACAAAACCATCGCCCTGATCATGAACAGTAGTGGCAGTAC
CGGATTGCCCAAGGGCGTAGCCCTACCGCACCGCACCGCTTGTGTCCGAT
TCAGTCATGCCCGCGACCCCATCTTCGGCAACCAGATCATCCCCGACACC
GCTATCCTCAGCGTGGTGCCATTTCACCACGGCTTCGGCATGTTCACCAC
GCTGGGCTACTTGATCTGCGGCTTTCGGGTCGTGCTCATGTACCGCTTCG
AGGAGGAGCTATTCTTGCGCAGCTTGCAAGACTATAAGATTCAATCTGCC
CTGCTGGTGCCCACACTATTTAGCTTCTTCGCTAAGAGCACTCTCATCGA
CAAGTACGACCTAAGCAACTTGCACGAGATCGCCAGCGGCGGGGCGCCGC
TCAGCAAGGAGGTAGGTGAGGCCGTGGCCAAACGCTTCCACCTACCAGGC
ATCCGCCAGGGCTACGGCCTGACAGAAACAACCAGCGCCATTCTGATCAC
CCCCGAAGGGGACGACAAGCCTGGCGCAGTAGGCAAGGTGGTGCCCTTCT
TCGAGGCTAAGGTGGTGGACTTGGACACAGGTAAGACACTGGGTGTGAAC
CAGCGCGGCGAGCTGTGCGTCCGTGGCCCCATGATCATGAGCGGCTACGT
TAACAACCCCGAGGCTACAAACGCTCTCATCGACAAGGACGGCTGGCTGC
ACAGCGGCGACATCGCCTACTGGGACGAGGACGAGCACTTCTTCATCGTG
GACCGGCTGAAGAGCCTGATCAAATACAAGGGCTACCAGGTAGCCCCAGC
CGAACTGGAGAGCATCCTGCTGCAACACCCCAACATCTTCGACGCCGGGG
TCGCCGGCCTGCCCGACGACGATGCCGGCGAGCTGCCCGCCGCAGTCGTC
GTGCTGGAACACGGTAAAACCATGACCGAGAAGGAGATCGTGGACTATGT
GGCCAGCCAGGTTACAACCGCCAAGAAGCTGCGCGGTGGTGTTGTGTTCG
TGGACGAGGTGCCTAAAGGACTGACCGGCAAGTTGGACGCCCGCAAGATC
CGCGAGATTCTCATTAAGGCCAAGAAGGGCGGCAAGATCGCCGTGGGCTC
CGGAGAGGGCAGAGGAAGTCTGCTAACATGCGGTGACGTCGAGGAGAATC
CTGGCCCAATGGTGAGCAAGGGCGAGGCAGTGATCAAGGAGTTCATGCGG
TTCAAGGTGCACATGGAGGGCTCCATGAACGGCCACGAGTTCGAGATCGA
GGGCGAGGGCGAGGGCCGCCCCTACGAGGGCACCCAGACCGCCAAGCTGA
AGGTGACCAAGGGTGGCCCCCTGCCCTTCTCCTGGGACATCCTGTCCCCT
CAGTTCATGTACGGCTCCAGGGCCTTCATCAAGCACCCCGCCGACATCCC
CGACTACTATAAGCAGTCCTTCCCCGAGGGCTTCAAGTGGGAGCGCGTGA
TGAACTTCGAGGACGGCGGCGCCGTGACCGTGACCCAGGACACCTCCCTG
GAGGACGGCACCCTGATCTACAAGGTGAAGCTCCGCGGCACCAACTTCCC
TCCTGACGGCCCCGTAATGCAGAAGAAGACAATGGGCTGGGAAGCGTCCA
CCGAGCGGTTGTACCCCGAGGACGGCGTGCTGAAGGGCGACATTAAGATG
GCCCTGCGCCTGAAGGACGGCGGCCGCTACCTGGCGGACTTCAAGACCAC
CTACAAGGCCAAGAAGCCCGTGCAGATGCCCGGCGCCTACAACGTCGACC
GCAAGTTGGACATCACCTCCCACAACGAGGACTACACCGTGGTGGAACAG
TACGAACGCTCCGAGGGCCGCCACTCCACCGGCGGCATGGACGAGCTGTA
CAAGTAAGCTGATCAGCCTCGATAAGATACATTGATGAGTTTGGACAAAC
CACAACTAGAATGCAGTGAAAAAAATGCTTTATTTGTGAAATTTGTGATG
CTATTGCTTTATTTGTAACCATTATAAGCTGCAATAAACAAGTTAAGGTC
GGGCAGGAAGAGGGCCTATTTCCCATGATTCCTTCATATTTGCATATACG
ATACAAGGCTGTTAGAGAGATAATTAGAATTAATTTGACTGTAAACACAA
AGATATTAGTACAAAATACGTGACGTAGAAAGTAATAATTTCTTGGGTAG
TTTGCAGTTTTAAAATTATGTTTTAAAATGGACTATCATATGCTTACCGT
AACTTGAAAGTATTTCGATTTCTTGGCTTTATATATCTTGTGGAAAGGAC
GAAACACCACTGCATGACCCAGATCAAcgaaTTGATCTGGGTCATGCAGT
TTTTTTAGGAACCCCTAGTGATGGAGTTGGCCACTCCCTCTCTGCGCGCT
CGCTCGCTCACTGAGGCCGGGCGACCAAAGGTCGCCCGACGCCCGGGCTT
TGCCCGGGCGGCCTCAGTGAGCGAGCGAGCGCGCAGCTGC

In some embodiments of any of the compositions described herein, the vector comprises or consists of pITR-CMV-Luc-T2A-GFP-U6-Hes1-KOP (SEQ ID NO: 79). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 79.

pITR-CMV-Luc2-T2A-GFP-U6-Hes1-KOP
(SEQ ID NO: 79)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctctagatcccatatatggagttccg
cgttacataacttacggtaaatggcccgcctggctgaccgcccaacgacccccgcccattgacgtcaataatgacgt
atgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcatta
tgcccagtacatgaccttatgggactttcctacttggcagtacatctacgtattagtcatcgctattaccatggtga
tgcggttttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgca
aatgggcggtaggcgtgtacggtgggaggtctatataagcagagctcgtttagtgaaccgtcagatcgcctggagac
gcatggaagatgccaaaaacattaagaagggcccagcgccattctacccactcgaagacgggaccgccggcgagcag
ctgcacaaagccatgaagcgctacgccctggtgcccggcaccatcgcctttaccgacgcacatatcgaggtggacat
tacctacgccgagtacttcgagatgagcgttcggctggcagaagctatgaagcgctatgggctgaatacaaaccatc
ggatcgtggtgtgcagcgagaatagcttgcagttcttcatgcccgtgttgggtgccctgttcatcggtgtggctgtg
gccccagctaacgacatctacaacgagcgcgagctgctgaacagcatgggcatcagccagcccaccgtcgtattcgt
gagcaagaaagggctgcaaaagatcctcaacgtgcaaaagaagctaccgatcatacaaaagatcatcatcatggata
gcaagaccgactaccagggcttccaaagcatgtacaccttcgtgacttcccatttgccacccggcttcaacgagtac
gacttcgtgcccgagagcttcgaccgggacaaaaccatcgccctgatcatgaacagtagtggcagtaccggattgcc
caagggcgtagccctaccgcaccgcaccgcttgtgtccgattcagtcatgcccgcgaccccatcttcggcaaccaga
tcatccccgacaccgctatcctcagcgtggtgccatttcaccacggcttcggcatgttcaccacgctgggctacttg
atctgcggctttcgggtcgtgctcatgtaccgcttcgaggaggagctattcttgcgcagcttgcaagactataagat
tcaatctgccctgctggtgcccacactatttagcttcttcgctaagagcactctcatcgacaagtacgacctaagca
acttgcacgagatcgccagcggcggggcgccgctcagcaaggaggtaggtgaggccgtggccaaacgcttccaccta
ccaggcatccgccagggctacggcctgacagaaacaaccagcgccattctgatcacccccgaaggggacgacaagcc
tggcgcagtaggcaaggtggtgcccttcttcgaggctaaggtggtggacttggacacaggtaagacactgggtgtga
accagcgcggcgagctgtgcgtccgtggccccatgatcatgagcggctacgttaacaaccccgaggctacaaacgct
ctcatcgacaaggacggctggctgcacagcggcgacatcgcctactgggacgaggacgagcacttcttcatcgtgga
ccggctgaagagcctgatcaaatacaagggctaccaggtagccccagccgaactggagagcatcctgctgcaacacc
ccaacatcttcgacgccggggtcgccggcctgcccgacgacgatgccggcgagctgcccgccgcagtcgtcgtgctg
gaacacggtaaaaccatgaccgagaaggagatcgtggactatgtggccagccaggttacaaccgccaagaagctgcg
cggtggtgttgtgttcgtggacgaggtgcctaaaggactgaccggcaagttggacgcccgcaagatccgcgagattc
tcattaaggccaagaagggcggcaagatcgccgtgggctccggagagggcagaggaagtctgctaacatgcggtgac
gtcgaggagaatcctggcccaatggtgagcaagggcgaggcagtgatcaaggagttcatgcggttcaaggtgcacat
ggagggctccatgaacggccacgagttcgagatcgagggcgagggcgagggccgcccctacgagggcacccagaccg
ccaagctgaaggtgaccaagggtggccccctgcccttctcctgggacatcctgtcccctcagttcatgtacggctcc
agggccttcatcaagcaccccgccgacatccccgactactataagcagtccttccccgagggcttcaagtgggagcg
cgtgatgaacttcgaggacggcggcgccgtgaccgtgacccaggacacctccctggaggacggcaccctgatctaca
aggtgaagctccgcggcaccaacttccctcctgacggccccgtaatgcagaagaagacaatgggctgggaagcgtcc
accgagcggttgtaccccgaggacggcgtgctgaagggcgacattaagatggccctgcgcctgaaggacggcggccg
ctacctggcggacttcaagaccacctacaaggccaagaagcccgtgcagatgcccggcgcctacaacgtcgaccgca
agttggacatcacctcccacaacgaggactacaccgtggtggaacagtacgaacgctccgagggccgccact’ccacc
ggcggcatggacgagctgtacaagtaagctgatcagcctcgataagatacattgatgagtttggacaaaccacaact
agaatgcagtgaaaaaaatgctttatttgtgaaatttgtgatgctattgctttatttgtaaccattataagctgcaa
taaacaagttaaggtcgggcaggaagagggcctatttcccatgattccttcatatttgcatatacgatacaaggctg
ttagagagataattagaattaatttgactgtaaacacaaagatattagtacaaaatacgtgacgtagaaagtaataa
tttcttgggtagtttgcagttttaaaattatgttttaaaatggactatcatatgcttaccgtaacttgaaagtattt
cgatttcttggctttatatatcttgtggaaaggacgaaacaccgcagctgatataatggagaacgaattctccatta
tatcagctgttttttaggaacccctagtgatggagttggccactccctctctgcgcgctcgctcgctcactgaggcc
gggcgaccaaaggtcgcccgacgcccgggctttgcccgggcggcctcagtgagcgagcgagcgcgcagctgc
Hes1-KOP
(SEQ ID NO: 80)
gcagctgatataatggagaa
SV40pA cDNA sequence
(SEQ ID NO: 70)
gctgatcagcctcgataagatacattgatgagtttggacaaaccacaactagaatgcagtgaaaaaaatgctttatt
tgtgaaatttgtgatgctattgctttatttgtaaccattataagctgcaataaacaagtt
U6 cDNA sequence
(SEQ ID NO: 71)
aaggtcgggcaggaagagggcctatttcccatgattccttcatatttgcatatacgatacaaggctgttagagagat
aattagaattaatttgactgtaaacacaaagatattagtacaaaatacgtgacgtagaaagtaataatttcttgggt
agtttgcagttttaaaattatgttttaaaatggactatcatatgcttaccgtaacttgaaagtatttcgatttcttg
gctttatatatcttgtggaaaggacgaaacacc
U6 cDNA sequence
(SEQ ID NO: 84)
CGGTGTTTCGTCCTTTCCACAAGATATATAAAGCCAAGAAATCGAAATACTTTCAAGTTACGGTAAGCATATGATAG
TCCATTTTAAAACATAATTTTAAAACTGCAAACTACCCAAGAAATTATTACTTTCTACGTCACGTATTTTGTACTAA
TATCTTTGTGTTTACAGTCAAATTAATTCTAATTATCTCTCTAACAGCCTTGTATCGTATATGCAAATATGAAGGAA
TCATGGGAAATAGGCCCTCTTCCTGCCCGACC
siRNA cDNA sequence
(SEQ ID NO: 72)
(N)₂₀CGAA(N)₂₀TTTTTT

In some embodiments of any of the compositions described herein, the vector comprises or consists of pITR-CMV-mScarlet-bGHpA (SEQ ID NO: 76). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 76.

pITR-CMV-mScarlet
(SEQ ID NO: 76)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctgtgatgcggttttggcagtacatc
aatgggcgtggatagcggtttgactcacggggatttccaagtctccaccccattgacgtcaatgggagtttgttttg
gcaccaaaatcaacgggactttccaaaatgtcgtaacaactccgccccattgacgcaaatgggcggtaggcgtgtac
ggtgggaggtctatataagcagagctcgtttagtgaaccgtcagatcgcctggagacgcgtgagcaagggcgaggca
gtgatcaaggagttcatgcggttcaaggtgcacatggagggctccatgaacggccacgagttcgagatcgagggcga
gggcgagggccgcccctacgagggcacccagaccgccaagctgaaggtgaccaagggtggccccctgcccttctcct
gggacatcctgtcccctcagttcatgtacggctccagggccttcatcaagcaccccgccgacatccccgactactat
aagcagtccttccccgagggcttcaagtgggagcgcgtgatgaacttcgaggacggcggcgccgtgaccgtgaccca
ggacacctccctggaggacggcaccctgatctacaaggtgaagctccgcggcaccaacttccctcctgacggccccg
taatgcagaagaagacaatgggctgggaagcgtccaccgagcggttgtaccccgaggacggcgtgctgaagggcgac
attaagatggccctgcgcctgaaggacggcggccgctacctggcggacttcaagaccacctacaaggccaagaagcc
cgtgcagatgcccggcgcctacaacgtcgaccgcaagttggacatcacctcccacaacgaggactacaccgtggtgg
aacagtacgaacgctccgagggccgccactccaccggcggcatggacgagctgtacaagtaagctgatcagcctcga
ctgtgccttctagttgccagccatctgttgtttgcccctcccccgtgccttccttgaccctggaaggtgccactccc
actgtcctttcctaataaaatgaggaaattgcatcgcattgtctgagtaggtgtcattctattctggggggtggggt
ggggcaggacagcaagggggaggattgggaagacaatagcaggcatgctggggatgcggtgggctctatggaggaac
ccctagtgatggagttggccactccctctctgcgcgctcgctcgctcactgaggccgggcgaccaaaggtcgcccga
cgcccgggctttgcccgggcggcctcagtgagcgagcgagcgcgcagctgcctgcagg

In some embodiments of any of the compositions described herein, the vector comprises or consists of pITR-CMV-mScarlet-DD-bGHpA (SEQ ID NO: 77). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 77.

pITR-CMV-mScarlet
(SEQ ID NO: 77)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctgtgatgcggttttggcagtacatc
aatgggcgtggatagcggtttgactcacggggatttccaagtctccaccccattgacgtcaatgggagtttgttttg
gcaccaaaatcaacgggactttccaaaatgtcgtaacaactccgccccattgacgcaaatgggcggtaggcgtgtac
ggtgggaggtctatataagcagagctcgtttagtgaaccgtcagatcgcctggagacgcgtgagcaagggcgaggca
gtgatcaaggagttcatgcggttcaaggtgcacatggagggctccatgaacggccacgagttcgagatcgagggcga
gggcgagggccgcccctacgagggcacccagaccgccaagctgaaggtgaccaagggtggccccctgcccttctcct
gggacatcctgtcccctcagttcatgtacggctccagggccttcatcaagcaccccgccgacatccccgactactat
aagcagtccttccccgagggcttcaagtgggagcgcgtgatgaacttcgaggacggcggcgccgtgaccgtgaccca
ggacacctccctggaggacggcaccctgatctacaaggtgaagctccgcggcaccaacttccctcctgacggccccg
taatgcagaagaagacaatgggctgggaagcgtccaccgagcggttgtaccccgaggacggcgtgctgaagggcgac
attaagatggccctgcgcctgaaggacggcggccgctacctggcggacttcaagaccacctacaaggccaagaagcc
cgtgcagatgcccggcgcctacaacgtcgaccgcaagttggacatcacctcccacaacgaggactacaccgtggtgg
aacagtacgaacgctccgagggccgccactccaccggcggcatggacgagctgtacaagtaaatcagtctgattgcg
gcgttagcggtagattacgttatcggcatggaaaacgccatgccgtggaacctgcctgccgatctcgcctggtttaa
acgcaacaccttaaataaacccgtgattatgggccgccatacctgggaatcaatcggtcgtccgttgccaggacgca
aaaatattatcctcagcagtcaaccgagtacggacgatcgcgtaacgtgggtgaagtcggtggatgaagccatcgcg
gcgtgtggtgacgtaccagaaatcatggtgattggcggcggtcgcgttattgaacagttcttgccaaaagcgcaaaa
actgtatctgacgcatatcgacgcagaagtggaaggcgacacccatttcccggattacgagccggatgactgggaat
cggtattcagcgaattccacgatgctgatgcgcagaactctcacagctattgctttgagattctggagcggcgagct
gatcagcctcgactgtgccttctagttgccagccatctgttgtttgcccctcccccgtgccttccttgaccctggaa
ggtgccactcccactgtcctttcctaataaaatgaggaaattgcatcgcattgtctgagtaggtgtcattctattct
ggggggtggggtggggcaggacagcaagggggaggattgggaagacaatagcaggcatgctggggatgcggtgggct
ctatggaggaacccctagtgatggagttggccactccctctctgcgcgctcgctcgctcactgaggccgggcgacca
aaggtcgcccgacgcccgggctttgcccgggcggcctcagtgagcgagcgagcgcgcagctgcctgcagg

In some embodiments of any of the compositions described herein, the vector comprises or consists of pITR-CMV-mScarlet (SEQ ID NO: 81). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 81.

pITR-CMV-mScarlet
(SEQ ID NO: 81)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctgcggccgcacgcgtctagatccca
tatatggagttccgcgttacataacttacggtaaatggcccgcctggctgaccgcccaacgacccccgcccattgac
gtcaataatgacgtatgttcccatagtaacgccaatagggactttccattgacgtcaatgggtggagtatttacggt
aaactgcccacttggcagtacatcaagtgtatcatatgccaagtacgccccctattgacgtcaatgacggtaaatgg
cccgcctggcattatgcccagtacatgaccttatgggactttcctacttggcagtacatctacgtattagtcatcgc
tattaccatggtgatgcggttttggcagtacatcaatgggcgtggatagcggtttgactcacggggatttccaagtc
tccaccccattgacgtcaatgggagtttgttttggcaccaaaatcaacgggactttccaaaatgtcgtaacaactcc
gccccattgacgcaaatgggcggtaggcgtgtacggtgggaggtctatataagcagagctcgtttagtgaaccgtca
gatcgcctggagacgcaccggtgccaccatgcctaagaagaagcggaaagtcggctccggcgtgagcaagggcgagg
cagtgatcaaggagttcatgcggttcaaggtgcacatggagggctccatgaacggccacgagttcgagatcgagggc
gagggcgagggccgcccctacgagggcacccagaccgccaagctgaaggtgaccaagggtggccccctgcccttctc
ctgggacatcctgtcccctcagttcatgtacggctccagggccttcatcaagcaccccgccgacatccccgactact
ataagcagtccttccccgagggcttcaagtgggagcgcgtgatgaacttcgaggacggcggcgccgtgaccgtgacc
caggacacctccctggaggacggcaccctgatctacaaggtgaagctccgcggcaccaacttccctcctgacggccc
cgtaatgcagaagaagacaatgggctgggaagcgtccaccgagcggttgtaccccgaggacggcgtgctgaagggcg
acattaagatggccctgcgcctgaaggacggcggccgctacctggcggacttcaagaccacctacaaggccaagaag
cccgtgcagatgcccggcgcctacaacgtcgaccgcaagttggacatcacctcccacaacgaggactacaccgtggt
ggaacagtacgaacgctccgagggccgccactccaccggcggcatggacgagctgtacaagtaataagagctcgctg
atcagcctcgactgtgccttctagttgccagccatctgttgtttgcccctcccccgtgccttccttgaccctggaag
gtgccactcccactgtcctttcctaataaaatgaggaaattgcatcgcattgtctgagtaggtgtcattctattctg
gggggtggggtggggcaggacagcaagggggaggattgggaagacaatagcaggcatgctggggatgcggtgggctc
tatggaagcttgaattcagctgacgtgcctcggaccgctaggaacccctagtgatggagttggccactccctctctg
cgcgctcgctcgctcactgaggccgggcgaccaaaggtcgcccgacgcccgggctttgcccgggcggcctcagtgag
cgagcgagcgcgcagctgcctgcaggggcgcctgatgcggtattttctccttacgcatctgtgcggtatttcacacc
gcatacgtcaaagcaaccatagtacgcgccctgtagcggcgcattaagcgcggcgggtgtggtggttacgcgcagcg
tgaccgctacacttgccagcgccctagcgcccgctcctttcgctttcttcccttcctttctcgccacgttcgccggc
tttccccgtcaagctctaaatcgggggctccctttagggttccgatttagtgctttacggcacctcgaccccaaaaa
acttgatttgggtgatggttcacgtagtgggccatcgccctgatagacggtttttcgccctttgacgttggagtcca
cgttctttaatagtggactcttgttccaaactggaacaacactcaaccctatctcgggctattcttttgatttataa
gggattttgccgatttcggcctattggttaaaaaatgagctgatttaacaaaaatttaacgcgaattttaacaaaat
attaacgtttacaattttatggtgcactctcagtacaatctgctctgatgccgcatagttaagccagccccgacacc
cgccaacacccgctgacgcgccctgacgggcttgtctgctcccggcatccgcttacagacaagctgtgaccgtctcc
gggagctgcatgtgtcagaggttttcaccgtcatcaccgaaacgcgcgagacgaaagggcctcgtgatacgcctatt
tttataggttaatgtcatgaacaataaaactgtctgcttacataaacagtaatacaaggggtgttatgagccatatt
caacgggaaacgtcgaggccgcgattaaattccaacatggatgctgatttatatgggtataaatgggctcgcgataa
tgtcgggcaatcaggtgcgacaatctatcgcttgtatgggaagcccgatgcgccagagttgtttctgaaacatggca
aaggtagcgttgccaatgatgttacagatgagatggtcagactaaactggctgacggaatttatgcctcttccgacc
atcaagcattttatccgtactcctgatgatgcatggttactcaccactgcgatccccggaaaaacagcattccaggt
attagaagaatatcctgattcaggtgaaaatattgttgatgcgctggcagtgttcctgcgccggttgcattcgattc
ctgtttgtaattgtccttttaacagcgatcgcgtatttcgtctcgctcaggcgcaatcacgaatgaataacggtttg
gttgatgcgagtgattttgatgacgagcgtaatggctggcctgttgaacaagtctggaaagaaatgcataaactttt
gccattctcaccggattcagtcgtcactcatggtgatttctcacttgataaccttatttttgacgaggggaaattaa
taggttgtattgatgttggacgagtcggaatcgcagaccgataccaggatcttgccatcctatggaactgcctcggt
gagttttctccttcattacagaaacggctttttcaaaaatatggtattgataatcctgatatgaataaattgcagtt
tcatttgatgctcgatgagtttttctaatctcatgaccaaaatcccttaacgtgagttttcgttccactgagcgtca
gaccccgtagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggcttcagca
gagcgcagataccaaatactgtccttctagtgtagccgtagttaggccaccacttcaagaactctgtagcaccgcct
acatacctcgctctgctaatcctgttaccagtggctgctgccagtggcgataagtcgtgtcttaccgggttggactc
aagacgatagttaccggataaggcgcagcggtcgggctgaacggggggttcgtgcacacagcccagcttggagcgaa
cgacctacaccgaactgagatacctacagcgtgagctatgagaaagcgccacgcttcccgaagggagaaaggcggac
aggtatccggtaagcggcagggtcggaacaggagagcgcacgagggagcttccagggggaaacgcctggtatcttta
tagtcctgtcgggtttcgccacctctgacttgagcgtcgatttttgtgatgctcgtcaggggggcggagcctatgga
aaaacgccagcaacgcggcctttttacggttcctggccttttgctggccttttgctcacatgt

In some embodiments of any of the compositions described herein, the vector comprises or consists of pITR-CMV-mScarlet-DD (SEQ ID NO: 82). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 82.

pITR-CMV-mScarlet-DD
(SEQ ID NO: 82)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctgcggccgcacgcgtctagatccca
tatatggagttccgcgttacataacttacggtaaatggcccgcctggctgaccgcccaacgacccccgcccattgac
gtcaataatgacgtatgttcccatagtaacgccaatagggactttccattgacgtcaatgggtggagtatttacggt
aaactgcccacttggcagtacatcaagtgtatcatatgccaagtacgccccctattgacgtcaatgacggtaaatgg
cccgcctggcattatgcccagtacatgaccttatgggactttcctacttggcagtacatctacgtattagtcatcgc
tattaccatggtgatgcggttttggcagtacatcaatgggcgtggatagcggtttgactcacggggatttccaagtc
tccacccdattgacgtcaatgggagtttgttttggcaccaaaatcaacgggactttccaaaatgtcgtaacaactcc
gccccattgacgcaaatgggcggtaggcgtgtacggtgggaggtctatataagcagagctcgtttagtgaaccgtca
gatcgcctggagacgcaccggtgccaccatgcctaagaagaagcggaaagtcggctccggcgtgagcaagggcgagg
cagtgatcaaggagttcatgcggttcaaggtgcacatggagggctccatgaacggccacgagttcgagatcgagggc
gagggcgagggccgcccctacgagggcacccagaccgccaagctgaaggtgaccaagggtggccccctgcccttctc
ctgggacatcctgtcccctcagttcatgtacggctccagggccttcatcaagcaccccgccgacatccccgactact
ataagcagtccttccccgagggcttcaagtgggagcgcgtgatgaacttcgaggacggcggcgccgtgaccgtgacc
caggacacctccctggaggacggcaccctgatctacaaggtgaagctccgcggcaccaacttccctcctgacggccc
cgtaatgcagaagaagacaatgggctgggaagcgtccaccgagcggttgtaccccgaggacggcgtgctgaagggcg
acattaagatggccctgcgcctgaaggacggcggccgctacctggcggacttcaagaccacctacaaggccaagaag
cccgtgcagatgcccggcgcctacaacgtcgaccgcaagttggacatcacctcccacaacgaggactacaccgtggt
ggaacagtacgaacgctccgagggccgccactccaccggcggcatggacgagctgtacaagggtaccatcagtctga
ttgcggcgttagcggtagattacgttatcggcatggaaaacgccatgccgtggaacctgcctgccgatctcgcctgg
tttaaacgcaacaccttaaataaacccgtgattatgggccgccatacctgggaatcaatcggtcgtccgttgccagg
acgcaaaaatattatcctcagcagtcaaccgagtacggacgatcgcgtaacgtgggtgaagtcggtggatgaagcca
tcgcggcgtgtggtgacgtaccagaaatcatggtgattggcggcggtcgcgttattgaacagttcttgccaaaagcg
caaaaactgtatctgacgcatatcgacgcagaagtggaaggcgacacccatttcccggattacgagccggatgactg
ggaatcggtattcagcgaattccacgatgctgatgcgcagaactctcacagctattgctttgagattctggagcggc
gataataagagctcgctgatcagcctcgactgtgccttctagttgccagccatctgttgtttgcccctcccccgtgc
cttccttgaccctggaaggtgccactcccactgtcctttcctaataaaatgaggaaattgcatcgcattgtctgagt
aggtgtcattctattctggggggtggggtggggcaggacagcaagggggaggattgggaagacaatagcaggcatgc
tggggatgcggtgggctctatggaagcttgaattcagctgacgtgcctcggaccgctaggaacccctagtgatggag
ttggccactccctctctgcgcgctcgctcgctcactgaggccgggcgaccaaaggtcgcccgacgcccgggctttgc
ccgggcggcctcagtgagcgagcgagcgcgcagctgcctgcaggggcgcctgatgcggtattttctccttacgcatc
tgtgcggtatttcacaccgcatacgtcaaagcaaccatagtacgcgccctgtagcggcgcattaagcgcggcgggtg
tggtggttacgcgcagcgtgaccgctacacttgccagcgccctagcgcccgctcctttcgctttcttcccttccttt
ctcgccacgttcgccggctttccccgtcaagctctaaatcgggggctccctttagggttccgatttagtgctttacg
gcacctcgaccccaaaaaacttgatttgggtgatggttcacgtagtgggccatcgccctgatagacggtttttcgcc
ctttgacgttggagtccacgttctttaatagtggactcttgttccaaactggaacaacactcaaccctatctcgggc
tattcttttgatttataagggattttgccgatttcggcctattggttaaaaaatgagctgatttaacaaaaatttaa
cgcgaattttaacaaaatattaacgtttacaattttatggtgcactctcagtacaatctgctctgatgccgcatagt
taagccagccccgacacccgccaacacccgctgacgcgccctgacgggcttgtctgctcccggcatccgcttacaga
caagctgtgaccgtctccgggagctgcatgtgtcagaggttttcaccgtcatcaccgaaacgcgcgagacgaaaggg
cctcgtgatacgcctatttttataggttaatgtcatgaacaataaaactgtctgcttacataaacagtaatacaagg
ggtgttatgagccatattcaacgggaaacgtcgaggccgcgattaaattccaacatggatgctgatttatatgggta
taaatgggctcgcgataatgtcgggcaatcaggtgcgacaatctatcgcttgtatgggaagcccgatgcgccagagt
tgtttctgaaacatggcaaaggtagcgttgccaatgatgttacagatgagatggtcagactaaactggctgacggaa
tttatgcctcttccgaccatcaagcattttatccgtactcctgatgatgcatggttactcaccactgcgatccccgg
aaaaacagcattccaggtattagaagaatatcctgattcaggtgaaaatattgttgatgcgctggcagtgttcctgc
gccggttgcattcgattcctgtttgtaattgtccttttaacagcgatcgcgtatttcgtctcgctcaggcgcaatca
cgaatgaataacggtttggttgatgcgagtgattttgatgacgagcgtaatggctggcctgttgaacaagtctggaa
agaaatgcataaacttttgccattctcaccggattcagtcgtcactcatggtgatttctcacttgataaccttattt
ttgacgaggggaaattaataggttgtattgatgttggacgagtcggaatcgcagaccgataccaggatcttgccatc
ctatggaactgcctcggtgagttttctccttcattacagaaacggctttttcaaaaatatggtattgataatcctga
tatgaataaattgcagtttcatttgatgctcgatgagtttttctaatctcatgaccaaaatcccttaacgtgagttt
tcgttccactgagcgtcagaccccgtagaaaagatcaaaggatcttcttgagatcctttttttctgcgcgtaatctg
ctgcttgcaaacaaaaaaaccaccgctaccagcggtggtttgtttgccggatcaagagctaccaactctttttccga
aggtaactggcttcagcagagcgcagataccaaatactgtccttctagtgtagccgtagttaggccaccacttcaag
aactctgtagcaccgcctacatacctcgctctgctaatcctgttaccagtggctgctgccagtggcgataagtcgtg
tcttaccgggttggactcaagacgatagttaccggataaggcgcagcggtcgggctgaacggggggttcgtgcacac
agcccagcttggagcgaacgacctacaccgaactgagatacctacagcgtgagctatgagaaagcgccacgcttccc
gaagggagaaaggcggacaggtatccggtaagcggcagggtcggaacaggagagcgcacgagggagcttccaggggg
aaacgcctggtatctttatagtcctgtcgggtttcgccacctctgacttgagcgtcgatttttgtgatgctcgtcag
gggggcggagcctatggaaaaacgccagcaacgcggcctttttacggttcctggccttttgctggccttttgctcac
atgt

In some embodiments of any of the compositions described herein, the vector comprises or consists of pITR-U6-shHES1-S5-CMV-3×FLAG-hATOH1-DD-T2A-hPOU4F3-U6-shHES1-S3 (SEQ ID NO: 83). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 83.

pITR-U6-shHES1-S5-CMV-3xFLAG-hATOH1-DD-T2A-hPOU4F3-U6-shHES1-S3
(SEQ ID NO: 83)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctgcggccgcaaaaaaactgcatgac
ccagatcaattcgttgatctgggtcatgcagtcggtgtttcgtcctttccacaagatatataaagccaagaaatcga
aatactttcaagttacggtaagcatatgatagtccattttaaaacataattttaaaactgcaaactacccaagaaat
tattactttctacgtcacgtattttgtactaatatctttgtgtttacagtcaaattaattctaattatctctctaac
agccttgtatcgtatatgcaaatatgaaggaatcatgggaaataggccctcttcctgcccgaccacgcgtctagatc
ccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttac
ggtaaactgcccacttggcagtacatcaagtgtatcatatgccaagtacgccccctattgacgtcaatgacggtaaa
tggcccgcctggcattatgcccagtacatgaccttatgggactttcctacttggcagtacatctacgtattagtcat
cgctattaccatggtgatgcggttttggcagtacatcaatgggcgtggatagcggtttgactcacggggatttccaa
gtctccaccccattgacgtcaatgggagtttgttttggcaccaaaatcaacgggactttccaaaatgtcgtaacaac
tccgccccattgacgcaaatgggcggtaggcgtgtacggtgggaggtctatataagcagagctaccggtgccaccat
ggattacaaggatgacgacgataaggactataaggacgatgatgacaaggactacaaagatgatgacgataaagtta
actcccgcctgctgcatgcagaagagtgggctgaagtgaaggagttgggagaccaccatcgccagccccagccgcat
catctcccgcaaccgccgccgccgccgcagccacctgcaactttgcaggcgagagagcatcccgtctacccgcctga
gctgtccctcctggacagcaccgacccacgcgcctggctggctcccactttgcagggcatctgcacggcacgcgccg
cccagtatttgctacattccccggagctgggtgcctcagaggccgctgcgccccgggacgaggtggacggccggggg
gagctggtaaggaggagcagcggcggtgccagcagcagcaagagccccgggccggtgaaagtgcgggaacagctgtg
caagctgaaaggcggggtggtggtagacgagctgggctgcagccgccaacgggccccttccagcaaacaggtgaatg
gggtgcagaagcagagacggctagcagccaacgccagggagcggcgcaggatgcatgggctgaaccacgccttcgac
cagctgcgcaatgttatcccgtcgttcaacaacgacaagaagctgtccaaatatgagaccctgcagatggcccaaat
ctacatcaacgccttgtccgagctgctacaaacgcccagcggaggggaacagccaccgccgcctccagcctcctgca
aaagcgaccaccaccaccttcgcaccgcggcctcctatgaagggggcgcgggcaacgcgaccgcagctggggctcag
caggcttccggagggagccagcggccgaccccgcccgggagttgccggactcgcttctcagccccagcttctgcggg
agggtactcggtgcagctggacgctctgcacttctcgactttcgaggacagcgccctgacagcgatgatggcgcaaa
agaatttgtctccttctctccccgggagcatcttgcagccagtgcaggaggaaaacagcaaaacttcgcctcggtcc
cacagaagcgacggggaattttccccccattcccattacagtgactcggatgaggcaagtgttaacatcagtctgat
tgcggcgttagcggtagattacgttatcggcatggaaaacgccatgccgtggaacctgcctgccgatctcgcctggt
ttaaacgcaacaccttaaataaacccgtgattatgggccgccatacctgggaatcaatcggtcgtccgttgccagga
cgcaaaaatattatcctcagcagtcaaccgagtacggacgatcgcgtaacgtgggtgaagtcggtggatgaagccat
cgcggcgtgtggtgacgtaccagaaatcatggtgattggcggcggtcgcgttattgaacagttcttgccaaaagcgc
aaaaactgtatctgacgcatatcgacgcagaagtggaaggcgacacccatttcccggattacgagccggatgactgg
gaatcggtattcagcgaattccacgatgctgatgcgcagaactctcacagctattgctttgagattctggagcggcg
aggatccggcgagggcagaggaagtctgctaacatgcggtgacgtcgaggagaatcctggcccaatgatggccatga
actccaagcagcctttcggcatgcacccggtgctgcaagaacccaaattctccagtctgcactctggctccgaggcc
atgcgccgagtctgtctcccagccccgcagctgcagggtaatatatttggaagctttgatgagagcctgctggcacg
cgccgaagctctggcggcggtggatatcgtctcccacggcaagaaccatccgttcaagcccgacgccacctaccata
ccatgagcagcgtgccctgcacgtccacttcgtccaccgtgcccatctcccacccagctgcgctcacctcacaccct
caccacgccgtgcaccagggcctcgaaggcgacctgctggagcacatctcgcccacgctgagtgtgagcggcctggg
cgctccggaacactcggtgatgcccgcacagatccatccacaccacctgggcgccatgggccacctgcaccaggcca
tgggcatgagtcacccgcacaccgtggcccctcatagcgccatgcctgcatgcctcagcgacgtggagtcagacccg
cgcgagctggaagccttcgccgagcgcttcaagcagcggcgcatcaagctgggggtgacccaggcggacgtgggcgc
ggctctggctaatctcaagatccccggcgtgggctcgctgagccaaagcaccatctgcaggttcgagtctctcactc
tctcgcacaacaacatgatcgctctcaagccggtgctccaggcctggttggaggaggccgaggccgcctaccgagag
aagaacagcaagccagagctcttcaacggcagcgaacggaagcgcaaacgcacgtccatcgcggcgccggagaagcg
ttcactcgaggcctatttcgctatccagccacgtccttcatctgagaagatcgcggccatcgctgagaaactggacc
ttaaaaagaacgtggtgagagtctggttctgcaaccagagacagaaacagaaacgaatgaagtattcggctgtccac
taaataataaaatatctttattttcattacatctgtgtgttggttttttgtgtgttaattaaaaaaaagaaagtcat
caaagcctatttcgataggctttgatgactttcggtgtttcgtcctttccacaagatatataaagccaagaaatcga
aatactttcaagttacggtaagcatatgatagtccattttaaaacataattttaaaactgcaaactacccaagaaat
tattactttctacgtcacgtattttgtactaatatctttgtgtttacagtcaaattaattctaattatctctctaac
agccttgtatcgtatatgcaaatatgaaggaatcatgggaaataggccctcttcctgcccgacccggaccgctagga
acccctagtgatggagttggccactccctctctgcgcgctcgctcgctcactgaggccgggcgaccaaaggtcgccc
gacgcccgggctttgcccgggcggcctcagtgagcgagcgagcgcgcagctgcctgcagg
U6 cDNA sequence
(SEQ ID NO: 91)
Ggtgtttcgtcctttccacaagatatataaagccaagaaatcgaaatactttcaagttacggtaagcatatgatagt
ccattttaaaacataattttaaaactgcaaactacccaagaaattattactttctacgtcacgtattttgtactaat
atctttgtgtttacagtcaaattaattctaattatctctctaacagccttgtatcgtatatgcaaatatgaaggaat
catgggaaataggccctcttcctgcccgacc
shHES1-1
(SEQ ID NO: 85)
cggtgtttcgtcctttccacaagatatataaagccaagaaatcgaaatactttcaagttacggtaagcatatgatag
tccattttaaaacataattttaaaactgcaaactacccaagaaattattactttctacgtcacgtattttgtactaa
tatctttgtgtttacagtcaaattaattctaattatctctctaacagccttgtatcgtatatgcaaatatgaaggaa
tcatgggaaataggccctcttcctgcccgacc
3x FLAG
(SEQ ID NO: 87)
atggattacaaggatgacgacgataaggactataaggacgatgatgacaaggactacaaagatgatgacgataaa
Human ATOH1 sequence
(SEQ ID NO: 87)
gttaactcccgcctgctgcatgcagaagagtgggctgaagtgaaggagttgggagaccaccatcgccagccccagcc
gcatcatctcccgcaaccgccgccgccgccgcagccacctgcaactttgcaggcgagagagcatcccgtctacccgc
ctgagctgtccctcctggacagcaccgacccacgcgcctggctggctcccactttgcagggcatctgcacggcacgc
gccgcccagtatttgctacattccccggagctgggtgcctcagaggccgctgcgccccgggacgaggtggacggccg
gggggagctggtaaggaggagcagcggcggtgccagcagcagcaagagccccgggccggtgaaagtgcgggaacagc
tgtgcaagctgaaaggcggggtggtggtagacgagctgggctgcagccgccaacgggccccttccagcaaacaggtg
aatggggtgcagaagcagagacggctagcagccaacgccagggagcggcgcaggatgcatgggctgaaccacgcctt
cgaccagctgcgcaatgttatcccgtcgttcaacaacgacaagaagctgtccaaatatgagaccctgcagatggccc
aaatctacatcaacgccttgtccgagctgctacaaacgcccagcggaggggaacagccaccgccgcctccagcctcc
tgcaaaagcgaccaccaccaccttcgcaccgcggcctcctatgaagggggcgcgggcaacgcgaccgcagctggggc
tcagcaggcttccggagggagccagcggccgaccccgcccgggagttgccggactcgcttctcagccccagcttctg
cgggagggtactcggtgcagctggacgctctgcacttctcgactttcgaggacagcgccctgacagcgatgatggcg
caaaagaatttgtctccttctctccccgggagcatcttgcagccagtgcaggaggaaaacagcaaaacttcgcctcg
gtcccacagaagcgacggggaattttccccccattcccattacagtgactcggatgaggcaagt
bGH PolyA sequence
(SEQ ID NO: 90)
ataataaaatatctttattttcattacatctgtgtgttggttttttgtgtg
shHE1-2
(SEQ ID NO: 92)
ggtgtttcgtcctttccacaagatatataaagccaagaaatcgaaatactttcaagttacggtaagcatatgatagt
ccattttaaaacataattttaaaactgcaaactacccaagaaattattactttctacgtcacgtattttgtactaat
atctttgtgtttacagtcaaattaattctaattatctctctaacagccttgtatcgtatatgcaaatatgaaggaat
catgggaaataggccctcttcctgcccgacc

In some embodiments of any of the compositions described herein, the vector comprises or consists of pITR-U6-shHES1-S5, hATOHessps-3×FLAG-hATOH1-T2A-hPOU4F3-US-shHES1-S3 (SEQ ID NO: 93). In some embodiments of any of the compositions described herein, the vector comprises a sequence that has at least 75% (e.g., at least 80%, at least 82%, at least 84%, at least 85%, at least 86%, at least 88%, at least 90%, at least 92%, at least 94%, at least 95%, at least 96%, at least 98%, at least 99%) sequence identity to SEQ ID NO: 93.

pITR-U6-shHES1-S5, hATOHessps-3xFLAG-hATOH1-T2A-hPOU4F3-US-shHES1-S3
(SEQ ID NO: 93)
cctgcaggcagctgcgcgctcgctcgctcactgaggccgcccgggcgtcgggcgacctttggtcgcccggcctcagt
gagcgagcgagcgcgcagagagggagtggccaactccatcactaggggttcctgcggccgcaaaaaaactgcatgac
ccagatcaattcgttgatctgggtcatgcagtcggtgtttcgtcctttccacaagatatataaagccaagaaatcga
aatactttcaagttacggtaagcatatgatagtccattttaaaacataattttaaaactgcaaactacccaagaaat
tattactttctacgtcacgtattttgtactaatatctttgtgtttacagtcaaattaattctaattatctctctaac
agccttgtatcgtatatgcaaatatgaaggaatcatgggaaataggccctcttcctgcccgaccacgcgtctatgga
gtttg'cataacaaacgtttggcagctcgctctcttacactccattaacaagctgtaacatatagctgcaggttgcta
taatctcattaatattttggaaacttgaatattgagtatttctgagtgctcattccccatatgccagccacttctgc
catgctgactggttcctttctctccattattagcaattagcttcttaccttccaaagtcagatccaaggtatccaag
atactagcaaaggaatcaactatgtgtgcaagttaagcatgcttaatatcacccaaacaaacaaagaggcagcattt
cttaaagtaatgaagatagataaatcgggttagtcctttgcgacactgctggtgctttctagagttttatatatttt
aagcagcttgctttatattctgtctttgcctcccaccccaccagcacttttatttgtggagggttttggctcgccac
actttgggaaacttatttgatttcacggagagctgaaggaagatcatttttggcaacagacaagtttaaacacgatt
tctatgggacattgctaactggggcccctaaggagaaaggggaaactgagcggagaatgggttaaatccttggaagc
aggggagaggcaggggaggagagaagtcggaggagtataaagaaaaggacaggaaccaagaagcgtgggggtggttt
gccgtaatgtgagtgtttcttaattagagaacggttgacaatagagggtctggcagaggctcctggccgcggtgcgg
agcgtctggagcggagcacgcgctgtcagctggtgagcgcactctcctttcaggcagctccccggggagctgtgcgg
ccacatttaacaccatcatcacccctccccggcctcctcaacctcggcctcctcctcgtcgacagccttccttggcc
cccaccagcagagctcacagtagcgagcgtctctcgccgtctcccgcactcggccggggcctctctcctcccccagc
tgcgcagcgggagccgccactgcccactgcacctcccagcaaccagcccagcacgcaaagaagctgcgcaaagttaa
agccaagcaatgccaaggggaggggaagctggaggcgggctttgagtggcttctgggcgcctggcgggtccagaatc
gcccagagccgcccgcggtcgtgcacatctgacccgagtcagcttgggcaccagccgagagccggctccgcaccgct
cccgcaccccagccgccggggtggtgacacacaccggagtcgaattacagccctgcaattaacatatgaatctgacg
aatttaaaagaaggaaaaaaaaaaaaaaacctgagcaggcttgggagtcctctgcacacaagaacttttctcggggt
gtaaaaactctttgattggctgctcgcacgcgcctgcccgcgccctccattggctgagaagacacgcgaccggcgcg
aggagggggttgggagaggagcggggggagactgagtggcgcgtgccgctttttaaaggggcgcagcgccttcagca
accggagaagcatagttgcacgcgacctggtgtgtgatctccgagtgggtgggggagggtcgaggagggaaaaaaaa
ataagacgttgcagaagagacccggaaagggccttttttttggttgagctggtgtcccagtgctgcctccgatcctg
agcctccgagcctttgcagtgcaaccggtgccaccatggattacaaggatgacgacgataaggactataaggacgat
gatgacaaggactacaaagatgatgacgataaagttaacatgtcccgcctgctgcatgcagaagagtgggctgaagt
gaaggagttgggagaccaccatcgccagccccagccgcatcatctcccgcaaccgccgccgccgccgcagccacctg
caactttgcaggcgagagagcatcccgtctacccgcctgagctgtccctcctggacagcaccgacccacgcgcctgg
ctggctcccactttgcagggcatctgcacggcacgcgccgcccagtatttgctacattccccggagctgggtgcctc
agaggccgctgcgccccgggacgaggtggacggccggggggagctggtaaggaggagcagcggcggtgccagcagca
gcaagagccccgggccggtgaaagtgcgggaacagctgtgcaagctgaaaggcggggtggtggtagacgagctgggc
tgcagccgccaacgggccccttccagcaaacaggtgaatggggtgcagaagcagagacggctagcagccaacgccag
ggagcggcgcaggatgcatgggctgaaccacgccttcgaccagctgcgcaatgttatcccgtcgttcaacaacgaca
agaagctgtccaaatatgagaccctgcagatggcccaaatctacatcaacgccttgtccgagctgctacaaacgccc
agcggaggggaacagccaccgccgcctccagcctcctgcaaaagcgaccaccaccaccttcgcaccgcggcctccta
tgaagggggcgcgggcaacgcgaccgcagctggggctcagcaggcttccggagggagccagcggccgaccccgcccg
ggagttgccggactcgcttctcagccccagcttctgcgggagggtactcggtgcagctggacgctctgcacttctcg
actttcgaggacagcgccctgacagcgatgatggcgcaaaagaatttgtctccttctctccccgggagcatcttgca
gccagtgcaggaggaaaacagcaaaacttcgcctcggtcccacagaagcgacggggaattttccccccattcccatt
acagtgactcggatgaggcaagtgttaacgagggcagaggaagtctgctaacatgcggtgacgtcgaggagaatcct
ggcccaatgatggccatgaactccaagcagcctttcggcatgcacccggtgctgcaagaacccaaattctccagtct
gcactctggctccgaggccatgcgccgagtctgtctcccagccccgcagctgcagggtaatatatttggaagctttg
atgagagcctgctggcacgcgccgaagctctggcggcggtggatatcgtctcccacggcaagaaccatccgttcaag
cccgacgccacctaccataccatgagcagcgtgccctgcacgtccacttcgtccaccgtgcccatctcccacccagc
tgcgctcacctcacaccctcaccacgccgtgcaccagggcctcgaaggcgacctgctggagcacatctcgcccacgc
tgagtgtgagcggcctgggcgctccggaacactcggtgatgcccgcacagatccatccacaccacctgggcgccatg
ggccacctgcaccaggccatgggcatgagtcacccgcacaccgtggcccctcatagcgccatgcctgcatgcctcag
cgacgtggagtcagacccgcgcgagctggaagccttcgccgagcgcttcaagcagcggcgcatcaagctgggggtga
cccaggcggacgtgggcgcggctctggctaatctcaagatccccggcgtgggctcgctgagccaaagcaccatctgc
aggttcgagtctctcactctctcgcacaacaacatgatcgctctcaagccggtgctccaggcctggttggaggaggc
cgaggccgcctaccgagagaagaacagcaagccagagctcttcaacggcagcgaacggaagcgcaaacgcacgtcca
tcgcggcgccggagaagcgttcactcgaggcctatttcgctatccagccacgtccttcatctgagaagatcgcggcc
atcgctgagaaactggaccttaaaaagaacgtggtgagagtctggttctgcaaccagagacagaaacagaaacgaat
gaagtattcggctgtccactaaataataaaatatctttattttcattacatctgtgtgttggttttttgtgtgttaa
ttaaaaaaaagaaagtcatcaaagcctatttcgataggctttgatgactttcggtgtttcgtcctttccacaagata
tataaagccaagaaatcgaaatactttcaagttacggtaagcatatgatagtccattttaaaacataattttaaaac
tgcaaactacccaagaaattattactttctacgtcacgtattttgtactaatatctttgtgtttacagtcaaattaa
ttctaattatctctctaacagccttgtatcgtatatgcaaatatgaaggaatcatgggaaataggccctcttcctgc
ccgacccggaccgctaggaacccctagtgatggagttggccactccctctctgcgcgctcgctcgctcactgaggcc
gggcgaccaaaggtcgcccgacgcccgggctttgcccgggcggcctcagtgagcgagcgagcgcgcagctgcctgca
gg
Human POU4F3 sequence
(SEQ ID NO: 95)
atgatggccatgaactccaagcagcctttcggcatgcacccggtgctgcaagaacccaaattctccagtctgcactc
tggctccgaggccatgcgccgagtctgtctcccagccccgcagctgcagggtaatatatttggaagctttgatgaga
gcctgctggcacgcgccgaagctctggcggcggtggatatcgtctcccacggcaagaaccatccgttcaagcccgac
gccacctaccataccatgagcagcgtgccctgcacgtccacttcgtccaccgtgcccatctcccacccagctgcgct
cacctcacaccctcaccacgccgtgcaccagggcctcgaaggcgacctgctggagcacatctcgcccacgctgagtg
tgagcggcctgggcgctccggaacactcggtgatgcccgcacagatccatccacaccacctgggcgccatgggccac
ctgcaccaggccatgggcatgagtcacccgcacaccgtggcccctcatagcgccatgcctgcatgcctcagcgacgt
ggagtcagacccgcgcgagctggaagccttcgccgagcgcttcaagcagcggcgcatcaagctgggggtgacccagg
cggacgtgggcgcggctctggctaatctcaagatccccggcgtgggctcgctgagccaaagcaccatctgcaggttc
gagtctctcactctctcgcacaacaacatgatcgctctcaagccggtgctccaggcctggttggaggaggccgaggc
cgcctaccgagagaagaacagcaagccagagctcttcaacggcagcgaacggaagcgcaaacgcacgtccatcgcgg
cgccggagaagcgttcactcgaggcctatttcgctatccagccacgtccttcatctgagaagatcgcggccatcgct
gagaaactggaccttaaaaagaacgtggtgagagtctggttctgcaaccagagacagaaacagaaacgaatgaagta
ttcggctgtccactaa

A variety of different methods known in the art can be used to introduce any of the AAV vectors disclosed herein into a primate cell (e.g., a supporting cell or a hair cell (e.g., an inner or outer cochlear hair cell)). Non-limiting examples of methods for introducing an AAV vector into a primate cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Skilled practitioners will appreciate that any of the AAV vectors described herein can be introduced into a primate cell (e.g., a hair cell or a supporting cell of the inner ear) by, for example, lipofection.

Various molecular biology techniques that can be used to correct a mutation(s) in an endogenous gene are also known in the art. Non-limiting examples of such techniques include site-directed mutagenesis, CRISPR (e.g., CRISPR/Cas9-induced knock-in mutations and CRISPR/Cas9-induced knock-out mutations), and TALENs. These methods can be used to correct the sequence of a defective endogenous gene present in a chromosome of a target cell (e.g., any of the exemplary cells described herein).

Any of the AAV vectors described herein can further include a control sequence, e.g., a control sequence selected from the group of a transcription initiation sequence, a transcription termination sequence, a promoter sequence, an enhancer sequence, an RNA splicing sequence, a polyadenylation (polyA) sequence, a Kozak consensus sequence, and a destabilizing domain sequence. Non-limiting examples of these control sequences are described herein. In some embodiments, a promoter can be a native promoter, a constitutive promoter, an inducible promoter, and/or a tissue-specific promoter.

Some embodiments of any of the compositions and kits described herein can include any combination of the AAV vectors described herein. Some embodiments of any of the methods described herein can include the use of any combination of the AAV vectors described herein.

Promoters

The term “promoter” means a DNA sequence recognized by enzymes/proteins in a primate cell required to initiate the transcription of a specific gene (e.g., a hair cell differentiation gene). A promoter typically refers to, e.g., a nucleotide sequence to which an RNA polymerase and/or any associated factor binds and at which transcription is initiated. Non-limiting examples of promoters are described herein. Additional examples of promoters are known in the art.

In some embodiments, an AAV vector encoding an N-terminal portion of a hair cell differentiation protein (e.g., a human hair cell differentiation protein) can include a promoter and/or an enhancer. The AAV vector encoding the N-terminal portion of the hair cell differentiation protein can include any of the promoters and/or enhancers described herein or known in the art.

In some embodiments, the promoter is an inducible promoter, a constitutive promoter, a primate cell promoter, a viral promoter, a chimeric promoter, an engineered promoter, a tissue-specific promoter, or any other type of promoter known in the art. In some embodiments, the promoter is a RNA polymerase II promoter, such as a primate RNA polymerase II promoter. In some embodiments, the promoter is a RNA polymerase III promoter, including, but not limited to, a H1 promoter, a human U6 promoter, a mouse U6 promoter, or a swine U6 promoter. The promoter will generally be one that is able to promote transcription in cochlear cells such as hair cells or supporting cells. In some examples, the promoter is a cochlea-specific promoter or a cochlea-oriented promoter.

A variety of promoters are known in the art that can be used herein. Non-limiting examples of promoters that can be used herein include: human EF1a, human cytomegalovirus (CMV) (GTGATGCGGTTTTGGCAGTACATCAATGGGCGTGGATAGCGGTTTGACTCACGGGG ATTTCCAAGTCTCCACCCCATTGACGTCAATGGGAGTTTGTTTTGGCACCAAAATCA ACGGGACTTTCCAAAATGTCGTAACAACTCCGCCCCATTGACGCAAATGGGCGGTA GGCGTGTACGGTGGGAGGTCTATATAAGCAGAGCTCGTTTAGTGAACCGTCAGATC GCCTGGAGACGC; SEQ ID NO: 53; U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g. human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone α gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007. In some embodiments, the promoter is the CMV immediate early promoter. In some embodiments, the promoter is a CAG promoter or a CAG/CBA promoter.

The term “constitutive” promoter refers to a nucleotide sequence that, when operably linked with a nucleic acid encoding a protein (e.g., a hair cell differentiation protein), causes RNA to be transcribed from the nucleic acid in a primate cell (e.g., a hair cell or a supporting cell of the inner ear) under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. U.S.A. 93:3346-3351, 1996), the tetracycline-repressible system (Gossen et al, Proc. Natl. Acad. Sci. U.S.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239-243, 1997) and Wang et al, Gene Ther. 4:432-441, 1997), and the rapamycin-inducible system (Magari et al. J. Clin. Invest. 100:2865-2872, 1997).

The term “tissue-specific” promoter refers to a promoter that is active only in certain specific cell types and/or tissues (e.g., transcription of a specific gene occurs only within cells expressing transcription regulatory proteins that bind to the tissue-specific promoter).

In some embodiments, the regulatory sequences impart tissue-specific gene expression capabilities. In some cases, the tissue-specific regulatory sequences bind tissue-specific transcription factors that induce transcription in a tissue-specific manner.

Exemplary tissue-specific promoters include but are not limited to the following: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a primate desmin (DES) promoter, an alpha-myosin heavy chain (a-MHC) promoter, and a cardiac Troponin T (cTnT) promoter. Additional exemplary promoters include Beta-actin promoter, hepatitis B virus core promoter (Sandig et al., Gene Ther. 3:1002-1009, 1996), alpha-fetoprotein (AFP) promoter (Arbuthnot et al., Hum. Gene Ther. 7:1503-1514, 1996), bone osteocalcin promoter (Stein et al., Mol. Biol. Rep. 24:185-196, 1997); bone sialoprotein promoter (Chen et al., J. Bone Miner. Res. 11:654-664, 1996), CD2 promoter (Hansal et al., J. Immunol. 161:1063-1068, 1998); immunoglobulin heavy chain promoter; T cell receptor alpha-chain promoter, neuronal such as neuron-specific enolase (NSE) promoter (Andersen et al., Cell. Mol. Neurobiol. 13:503-515, 1993), neurofilament light-chain gene promoter (Piccioli et al., Proc. Natl. Acad. Sci. U.S.A. 88:5611-5615, 1991), and the neuron-specific vgf gene promoter (Piccioli et al., Neuron 15:373-384, 1995).

In some embodiments, the tissue-specific promoter is a cochlea-specific promoter. In some embodiments, the tissue-specific promoter is a cochlear hair cell-specific promoter. Non-limiting examples of cochlear hair cell-specific promoters include but are not limited to: a ATOH1 promoter, a ATOH1 3′-enhancer, a POU4F3 promoter, a LHX3 promoter, a MYO7A promoter, a MYO6 promoter, a CHRNA9 promoter, and a CHRNA10 promoter. In some embodiments, the promoter is an outer hair cell-specific promoter such as a SLC26A5 promoter or an OCM promoter. See, e.g., Zheng et al., Nature 405:149-155, 2000; Tian et al. Dev. Dyn. 231:199-203, 2004; and Ryan et al., Adv. Otorhinolaryngol. 66: 99-115, 2009.

In some embodiments of any of the AAV vectors described herein, the AAV vector includes a human ATOH1 enhancer-promoter (SEQ ID NO: 94).

Human ATOH1 enhancer-promoter
(SEQ ID NO: 94)
ctatggagtttgcataacaaacgtttggcagctcgctctcttacactccattaacaagctgtaacatatagctgcag
gttgctataatctcattaatattttggaaacttgaatattgagtatttctgagtgctcattccccatatgccagcca
cttctgccatgctgactggttcctttctctccattattagcaattagcttcttaccttccaaagtcagatccaaggt
atccaagatactagcaaaggaatcaactatgtgtgcaagttaagcatgcttaatatcacccaaacaaacaaagaggc
agcatttcttaaagtaatgaagatagataaatcgggttagtcctttgcgacactgctggtgctttctagagttttat
atattttaagcagcttgctttatattctgtctttgcctcccaccccaccagcacttttatttgtggagggttttggc
tcgccacactttgggaaacttatttgatttcacggagagctgaaggaagatcatttttggcaacagacaagtttaaa
cacgatttctatgggacattgctaactggggcccctaaggagaaaggggaaactgagcggagaatgggttaaatcct
tggaagcaggggagaggcaggggaggagagaagtcggaggagtataaagaaaaggacaggaaccaagaagcgtgggg
gtggtttgccgtaatgtgagtgtttcttaattagagaacggttgacaatagagggtctggcagaggctcctggccgc
ggtgcggagcgtctggagcggagcacgcgctgtcagctggtgagcgcactctcctttcaggcagctccccggggagc
tgtgcggccacatttaacaccatcatcacccctccccggcctcctcaacctcggcctcctcctcgtcgacagccttc
cttggcccccaccagcagagctcacagtagcgagcgtctctcgccgtctcccgcactcggccggggcctctctcctc
ccccagctgcgcagcgggagccgccactgcccactgcacctcccagcaaccagcccagcacgcaaagaagctgcgca
aagttaaagccaagcaatgccaaggggaggggaagctggaggcgggctttgagtggcttctgggcgcctggcgggtc
cagaatcgcccagagccgcccgcggtcgtgcacatctgacccgagtcagcttgggcaccagccgagagccggctccg
caccgctcccgcaccccagccgccggggtggtgacacacaccggagtcgaattacagccctgcaattaacatatgaa
tctgacgaatttaaaagaaggaaaaaaaaaaaaaaacctgagcaggcttgggagtcctctgcacacaagaacttttc
tcggggtgtaaaaactctttgattggctgctcgcacgcgcctgcccgcgccctccattggctgagaagacacgcgac
cggcgcgaggagggggttgggagaggagcggggggagactgagtggcgcgtgccgctttttaaaggggcgcagcgcc
ttcagcaaccggagaagcatagttgcacgcgacctggtgtgtgatctccgagtgggtgggggagggtcgaggaggga
aaaaaaaataagacgttgcagaagagacccggaaagggccttttttttggttgagctggtgtcccagtgctgcctcc
gatcctgagcctccgagcctttgcagtgcaa

Enhancers and 5′ Cap

In some instances, an AAV vector can include a promoter sequence and/or an enhancer sequence. The term “enhancer” refers to a nucleotide sequence that can increase the level of transcription of a nucleic acid encoding a protein of interest (e.g., a hair cell differentiation protein). Enhancer sequences (50-1500 basepairs in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer (CTAGATCCCATATATGGAGTTCCGCGTTACATAACTTACGGTAAATGGCCCGCCTGG CTGACCGCCCAACGACCCCCGCCCATTGACGTCAATAATGACGTATGTTCCCATAGT AACGCCAATAGGGACTTTCCATTGACGTCAATGGGTGGAGTATTTACGGTAAACTGC CCACTTGGCAGTACATCAAGTGTATCATATGCCAAGTACGCCCCCTATTGACGTCAA TGACGGTAAATGGCCCGCCTGGCATTATGCCCAGTACATGACCTTATGGGACTTTCC TACTTGGCAGTACATCTACGTATTAGTCATCGCTATTACCATG; SEQ ID NO: 52), and a SV40 enhancer.

In some embodiments of any of the AAV vectors described herein, the AAV vector includes a CMV enhancer-promoter sequence (SEQ ID NO: 96)

CMV enhancer-promoter sequence
(SEQ ID NO: 96)
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGGTGATGCGGTTTTGGCAGTACATCAATGGGCGTGGATAG
CGGTTTGACTCACGGGGATTTCCAAGTCTCCACCCCATTGACGTCAATG
GGAGTTTGTTTTGGCACCAAAATCAACGGGACTTTCCAAAATGTCGTAA
CAACTCCGCCCCATTGACGCAAATGGGCGGTAGGCGTGTACGGTGGGAG
GTCTATATAAGCAGAGCT

Poly(A) Sequences

In some embodiments, any of the AAV vectors provided herein can include a poly(A) sequence. Most nascent eukaryotic mRNAs possess a poly(A) tail at their 3′ end which is added during a complex process that includes cleavage of the primary transcript and a coupled polyadenylation reaction (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some embodiments, the poly(A) sequence is positioned 3′ to the nucleic acid sequence encoding the C-terminus of the hair cell differentiation protein or a protein of interest (e.g., a Cas9 endonuclease, e.g., a SaCas9 endonuclease (e.g., any of the SaCas9 endonucleases described herein), a reporter protein (e.g., a GFP protein, a mScarlet protein)).

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, the poly(A) tail is added onto transcripts that contain a specific sequence, the polyadenylation signal or “poly(A) sequence.” The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A) sequence” is a sequence that triggers the endonuclease cleavage of an mRNA and the additional of a series of adenosines to the 3′ end of the cleaved mRNA.

There are several poly(A) sequences that can be used, including those derived from bovine growth hormone (bgh) (Woychik et al., Proc. Natl. Acad. Sci. U.S.A. 81(13):3944-3948, 1984; U.S. Pat. No. 5,122,458), mouse-β-globin, mouse-α-globin (Orkin et al., EMBO J. 4(2):453-456, 1985; Thein et al., Blood 71(2):313-319, 1988), human collagen, polyoma virus (Batt et al., Mol. Cell Biol. 15(9):4783-4790, 1995), the Herpes simplex virus thymidine kinase gene (HSV TK), IgG heavy-chain gene polyadenylation signal (US 2006/0040354), human growth hormone (hGH) (Szymanski et al., Mol. Therapy 15(7):1340-1347, 2007), the group of SV40 poly(A) sites, such as the SV40 late and early poly(A) site (Schek et al., Mol. Cell Biol. 12(12):5386-5393, 1992).

The poly(A) sequence can be a sequence of AATAAA. The AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, the poly(A) sequence can be a synthetic polyadenylation site (see, e.g., the pCl-neo expression vector of Promega which is based on Levitt el al, Genes Dev. 3(7):1019-1025, 1989). In some embodiments, the poly(A) sequence is the polyadenylation signal of soluble neuropilin-1 (sNRP) (AAATAAAATACGAAATG) (see, e.g., WO 05/073384). Additional examples of poly(A) sequences are known in the art.

In some embodiments, the poly(A) sequence is a bGHpA sequence

(GCTGATCAGCCTCGACTGTGCCTTCTAGTTGCCAGCCATCTGTTGTTT
GCCCCTCCCCCGTGCCTTCCTTGACCCTGGAAGGTGCCACTCCCACTGT
CCTTTCCTAATAAAATGAGGAAATTGCATCGCATTGTCTGAGTAGGTGT
CATTCTATTCTGGGGGGTGGGGTGGGGCAGGACAGCAAGGGGGAGGATT
GGGAAGACAATAGCAGGCATGCTGGGGATGCGGTGGGCTCTATGG;
SEQ ID NO: 56).

Internal Ribosome Entry Site (IRES)

In some embodiments, an AAV vector encoding the C-terminus of the hair cell differentiation protein can include a polynucleotide internal ribosome entry site (IRES). An IRES sequence is used to produce more than one polypeptide from a single gene transcript. An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into the vector that encodes the C-terminus of a hair cell differentiation protein is the foot and mouth disease virus (FMDV) 2A sequence. In some embodiments, the IRES sequence that is incorporated into the vector that encodes the C-terminal portion of a protein of interest (e.g., a Cas9 endonuclease, e.g., a SaCas9 endonuclease (e.g., any of the SaCas9 endonucleases described herein)) is the FMDV 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy vectors (AAV and retroviruses) (Ryan et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11:1921-1931, 2000; and Klump et al., Gene Therapy 8:811-817, 2001).

Destabilizing Domain (DD)

Any of the AAV vectors provided herein can optionally include a sequence encoding a destabilizing domain (“a destabilizing sequence”) for temporal control of protein expression. Non-limiting examples of destabilizing sequences include sequences encoding: a FK506 sequence, a dihydrofolate reductase (DHFR) sequence. An exemplary DHFR destabilizing sequence is: MISLIAALAVDYVIGMENAMPWNLPADLAWFKRNTLNKPVIMGRHTWESIGRPLPGRK NIILSSQPSTDDRVTWVKSVDEAIAACGDVPEIMVIGGGRVIEQFLPKAQKLYLTHIDAEV EGDTHFPDYEPDDWESVFSEFHDADAQNSHSYCFEILERR (SEQ ID NO: 48). An exemplary DHFR destabilizing domain sequence is

(SEQ ID NO: 59)
GGTACCATCAGTCTGATTGCGGCGTTAGCGGTAGATTACGTTATCGGCA
TGGAAAACGCCATGCCGTGGAACCTGCCTGCCGATCTCGCCTGGTTTAA
ACGCAACACCTTAAATAAACCCGTGATTATGGGCCGCCATACCTGGGAA
TCAATCGGTCGTCCGTTGCCAGGACGCAAAAATATTATCCTCAGCAGTC
AACCGAGTACGGACGATCGCGTAACGTGGGTGAAGTCGGTGGATGAAGC
CATCGCGGCGTGTGGTGACGTACCAGAAATCATGGTGATTGGCGGCGGT
CGCGTTATTGAACAGTTCTTGCCAAAAGCGCAAAAACTGTATCTGACGC
ATATCGACGCAGAAGTGGAAGGCGACACCCATTTCCCGGATTACGAGCC
GGATGACTGGGAATCGGTATTCAGCGAATTCCACGATGCTGATGCGCAG
AACTCTCACAGCTATTGCTTTGAGATTCTGGAGCGGCGATAA.

In some embodiments of any of the AAV vectors described herein, the AAV vector includes a destabilizing domain (SEQ ID NO: 88).

Destabilizing domain
(SEQ ID NO: 88)
Atcagtctgattgcggcgttagcggtagattacgttatcggcatggaaaacgccatgccgtggaacctgcctgccga
tctcgcctggtttaaacgcaacaccttaaataaacccgtgattatgggccgccatacctgggaatcaatcggtcgtc
cgttgccaggacgcaaaaatattatcctcagcagtcaaccgagtacggacgatcgcgtaacgtgggtgaagtcggtg
gatgaagccatcgcggcgtgtggtgacgtaccagaaatcatggtgattggcggcggtcgcgttattgaacagttctt
gccaaaagcgcaaaaactgtatctgacgcatatcgacgcagaagtggaaggcgacacccatttcccggattacgagc
cggatgactgggaatcggtattcagcgaattccacgatgctgatgcgcagaactctcacagctattgctttgagatt
ctggagcggcga

Additional examples of destabilizing sequences are known in the art. In some embodiments, the destabilizing sequence is a FK506- and rapamycin-binding protein (FKBP12) sequence, and the stabilizing ligand is Shield-1 (Shld1) (Banaszynski et al. (2012) Cell 126(5): 995-1004). An exemplary FKBP12 destabilizing sequence is: MGVEKQVIRPGNGPKPAPGQTVTVHCTGFG KDGDLSQKFWSTKDEGQKPFSFQIGKGAVIKGWDEGVIGMQIGEVARLRCSSDYAYGA GGFPAWGIQPNSVLDFEIEVLSVQ (SEQ ID NO: 49). In some embodiments, the destabilizing sequence is a DHFR sequence, and the stabilizing ligand is trimethoprim (TMP) (Iwamoto et al. (2010) Chem Biol 17:981-988).

In the absence of a stabilizing ligand, the protein sequence operatively linked to the destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the destabilizing sequence is a FKBP12 sequence, and the presence of an AAV vector carrying the FKBP12 gene in a primate cell (e.g., a supporting cochlear outer hair cell) is detected by western blotting. In some embodiments, the destabilizing sequence can be used to verify the temporally-specific activity of any of the AAV vectors described herein.

In some embodiments of any of the AAV vectors described herein, the AAV vector comprising the C-terminal portion of a hair cell differentiation gene, the vector further includes a destabilizing sequence 3′ of the C-terminal portion of the hair cell differentiation gene. In some embodiments of the AAV vector including a sequence encoding the C-terminal portion of an ATOH1 protein, the vector further comprises a sequence encoding a destabilizing domain (DD) (e.g., any of the destabilizing domain described herein).

Reporter Sequences/Detectable Marker Genes

Any of the AAV vectors provided herein can optionally include a sequence encoding a reporter protein or a detectable marker (“a reporter sequence” or “a detectable marker gene”). Non-limiting examples of reporter sequences or detectable marker genes include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences or detectable markers are known in the art. When associated with regulatory elements which drive their expression, the reporter sequence or detectable marker gene can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the reporter sequence or detectable marker gene is a 3× Flag sequence (GATTACAAGGATGACGACGATAAGGACTATAAGGACGATGATGACAAGGACTACA AAGATGATGACGATAAAGGATCCGGC; SEQ ID NO: 62). In some embodiments, the reporter sequence or detectable marker gene is a luciferase sequence

(ATGGAAGATGCCAAAAACATTAAGAAGGGCCCAGCGCCATTCTACCCACTCGAAG
ACGGGACCGCCGGCGAGCAGCTGCACAAAGCCATGAAGCGCTACGCCCTGGTGCCC
GGCACCATCGCCTTTACCGACGCACATATCGAGGTGGACATTACCTACGCCGAGTAC
TTCGAGATGAGCGTTCGGCTGGCAGAAGCTATGAAGCGCTATGGGCTGAATACAAA
CCATCGGATCGTGGTGTGCAGCGAGAATAGCTTGCAGTTCTTCATGCCCGTGTTGGG
TGCCCTGTTCATCGGTGTGGCTGTGGCCCCAGCTAACGACATCTACAACGAGCGCGA
GCTGCTGAACAGCATGGGCATCAGCCAGCCCACCGTCGTATTCGTGAGCAAGAAAG
GGCTGCAAAAGATCCTCAACGTGCAAAAGAAGCTACCGATCATACAAAAGATCATC
ATCATGGATAGCAAGACCGACTACCAGGGCTTCCAAAGCATGTACACCTTCGTGACT
TCCCATTTGCCACCCGGCTTCAACGAGTACGACTTCGTGCCCGAGAGCTTCGACCGG
GACAAAACCATCGCCCTGATCATGAACAGTAGTGGCAGTACCGGATTGCCCAAGGG
CGTAGCCCTACCGCACCGCACCGCTTGTGTCCGATTCAGTCATGCCCGCGACCCCAT
CTTCGGCAACCAGATCATCCCCGACACCGCTATCCTCAGCGTGGTGCCATTTCACCA
CGGCTTCGGCATGTTCACCACGCTGGGCTACTTGATCTGCGGCTTTCGGGTCGTGCTC
ATGTACCGCTTCGAGGAGGAGCTATTCTTGCGCAGCTTGCAAGACTATAAGATTCAA
TCTGCCCTGCTGGTGCCCACACTATTTAGCTTCTTCGCTAAGAGCACTCTCATCGACA
AGTACGACCTAAGCAACTTGCACGAGATCGCCAGCGGCGGGGCGCCGCTCAGCAAG
GAGGTAGGTGAGGCCGTGGCCAAACGCTTCCACCTACCAGGCATCCGCCAGGGCTA
CGGCCTGACAGAAACAACCAGCGCCATTCTGATCACCCCCGAAGGGGACGACAAGC
CTGGCGCAGTAGGCAAGGTGGTGCCCTTCTTCGAGGCTAAGGTGGTGGACTTGGAC
ACAGGTAAGACACTGGGTGTGAACCAGCGCGGCGAGCTGTGCGTCCGTGGCCCCAT
GATCATGAGCGGCTACGTTAACAACCCCGAGGCTACAAACGCTCTCATCGACAAGG
ACGGCTGGCTGCACAGCGGCGACATCGCCTACTGGGACGAGGACGAGCACTTCTTC
ATCGTGGACCGGCTGAAGAGCCTGATCAAATACAAGGGCTACCAGGTAGCCCCAGC
CGAACTGGAGAGCATCCTGCTGCAACACCCCAACATCTTCGACGCCGGGGTCGCCG
GCCTGCCCGACGACGATGCCGGCGAGCTGCCCGCCGCAGTCGTCGTGCTGGAACAC
GGTAAAACCATGACCGAGAAGGAGATCGTGGACTATGTGGCCAGCCAGGTTACAAC
CGCCAAGAAGCTGCGCGGTGGTGTTGTGTTCGTGGACGAGGTGCCTAAAGGACTGA
CCGGCAAGTTGGACGCCCGCAAGATCCGCGAGATTCTCATTAAGGCCAAGAAGGGC
GGCAAGATCGCCGTGGGCTCCGGA; SEQ ID NO: 69).

In some embodiments, the reporter sequence or detectable marker gene is the LacZ gene, and the presence of a vector carrying the LacZ gene in a primate cell (e.g., a supporting cochlear outer hair cell) is detected by assays for beta-galactosidase activity. In other embodiments, the reporter sequence or detectable marker gene is a fluorescent protein (e.g., green fluorescent protein) or luciferase, the presence of a vector carrying the fluorescent protein or luciferase in a primate cell (e.g., a supporting cochlear outer hair cell) may be measured by fluorescent techniques (e.g., fluorescent microscopy or FACS) or light production in a luminometer (e.g., a spectrophotometer or an IVIS imaging instrument). In some embodiments, the reporter sequence or detectable marker gene can be used to verify the tissue-specific targeting capabilities and tissue-specific promoter regulatory activity of any of the vectors described herein.

Flanking Regions Untranslated Regions (UTRs)

In some embodiments, any of the AAV vectors described herein (e.g., any of the at least two different vectors) can include an untranslated region. In some embodiments, an AAV vector can includes a 5′ UTR or a 3′ UTR.

Untranslated regions (UTRs) of a gene are transcribed but not translated. The 5′ UTR starts at the transcription start site and continues to the start codon but does not include the start codon. The 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into any of the vectors, compositions, kits, or methods as described herein to enhance the stability of a hair cell differentiation protein or of a protein of interest (e.g., a Cas9 endonuclease, e.g., a SaCas9 endonuclease (e.g., any of the SaCas9 endonucleases described herein), a reporter protein (e.g., a GFP protein, a mScarlet protein).

Natural 5′ UTRs include a sequence that plays a role in translation initiation. They harbor signatures like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), which is followed by another “G”. The 5′ UTR have also been known, e.g., to form secondary structures that are involved in elongation factor binding.

For example, in some embodiments, a 5′ UTR is included in any of the AAV vectors described herein. Non-limiting examples of 5′ UTRs including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA.

In some embodiments, a 5′ UTR from a mRNA that is transcribed by a cell in the cochlea can be included in any of the vectors, compositions, kits, and methods described herein.

3′ UTRs are known to have stretches of adenosines and uridines embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a hair cell differentiation protein. In other embodiments, AREs can be removed or mutated to increase the intracellular stability and thus increase translation and production of a hair cell differentiation protein.

In other embodiments, non-UTR sequences may be incorporated into the 5′ or 3′ UTRs. In some embodiments, introns or portions of intron sequences may be incorporated into the flanking regions of the polynucleotides in any of the vectors, compositions, kits, and methods provided herein. Incorporation of intronic sequences may increase protein production as well as mRNA levels.

### Inhibitory Nucleic Acids

Some embodiments of the compositions provided herein include a single AAV vector that encodes an inhibitory nucleic acid that decreases the expression of a hair cell differentiation-suppressing protein in a primate cell (e.g., a hair cell or a supporting cell of the inner ear). Inhibitory nucleic acids include, e.g., siRNA, shRNA, antisense nucleic acids, and ribozymes.

Non-limiting examples of siRNAs that can decrease the expression of a hair cell differentiation-suppressing protein in a primate cell (e.g., a hair cell or a supporting cell of the inner ear) are described herein. An inhibitory nucleic acid can be, e.g., a chemically-modified siRNAs or a vector-driven expression of short hairpin RNA (shRNA) that are then cleaved to siRNA. In some examples, an inhibitory nucleic acid can be a dsRNA (e.g., siRNA) including 16-30 nucleotides, e.g., 16, 17, 18, 19, 20, 21, 22, 23, 24, 25, 26, 27, 28, 29, or 30 nucleotides in each strand, where one of the strands is substantially identical, e.g., at least 80% (or more, e.g., 85%, 90%, 95%, or 100%) identical, e.g., having 3, 2, 1, or 0 mismatched nucleotide(s), to a target region in the hair cell differentiation-suppressing mRNA, and the other strand is complementary to the first strand. dsRNA molecules can be designed using methods known in the art, e.g., Dharmacon.com (see, siDESIGN CENTER) or “The siRNA User Guide,” available on the Internet at mpibpc.gwdg.de/abteilungen/100/105/sirna.html website.

Several methods for expressing siRNA duplexes within cells from a vector to achieve long-term target gene suppression in cells are known in the art, e.g., including vectors that use a mammalian Pol III promoter system (e.g., H1 or U6/snRNA promoter systems (Tuschl, Nature Biotechnol., 20:440-448, 2002) to express functional double-stranded siRNAs; (Bagella et al., J. Cell. Physiol., 177:206-213, 1998; Lee et al., Nature Biotechnol., 20:500-505, 2002; Paul et al., Nature Biotechnol., 20:505-508, 2002; Yu et al., Proc. Natl. Acad. Sci. U.S.A., 99(9):6047-6052, 2002; Sui et al., Proc. Natl. Acad. Sci. U.S.A. 99(6):5515-5520, 2002). Transcriptional termination by RNA Pol III occurs at runs of four consecutive T residues in the DNA template, and can be used to provide a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998, supra; Lee et al., 2002, supra; Paul et al., 2002, supra; Yu et al., 2002, supra; Sui et al., 2002, supra).

Animal cells express a range of noncoding RNAs of approximately 22 nucleotides termed micro RNA (miRNAs) and can regulate gene expression at the post transcriptional or translational level during animal development. miRNAs are excised from an approximately 70 nucleotide precursor RNA stem-loop. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng, Mol. Cell, 9:1327-1333, 2002). When expressed by DNA vectors containing polymerase III promoters, micro-RNA designed hairpins can silence gene expression (McManus, RNA 8:842-850, 2002).

In some examples, an inhibitory nucleic acid can be an antisense nucleic acid molecules, i.e., nucleic acid molecules whose nucleotide sequence is complementary to all or part of an mRNA encoding a hair cell differentiation-suppressing protein. An antisense nucleic acid molecule can be antisense to all or part of a non-coding region of the coding strand of a nucleotide sequence encoding a hair cell differentiation-suppressing protein. The non-coding regions (“5′ and 3′ untranslated regions”) are the 5′ and 3′ sequences that flank the coding region and are not translated into amino acids. Based upon the sequences disclosed herein, one of skill in the art can easily choose and synthesize any of a number of appropriate antisense molecules to target a hair cell differentiation-suppressing gene described herein. For example, a “gene walk” comprising a series of oligonucleotides of 15-30 nucleotides spanning the length of a nucleic acid (e.g., a hair cell differentiation-suppressing mRNA) can be prepared, followed by testing for inhibition of expression of the gene. Optionally, gaps of 5-10 nucleotides can be left between the oligonucleotides to reduce the number of oligonucleotides synthesized and tested.

An antisense oligonucleotide can be, for example, about 5, 10, 15, 20, 25, 30, 35, 40, 45, or 50 nucleotides or more in length.

In some embodiments, the inhibitory nucleic acid can be a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature, 334:585-591, 1988)) can be used to catalytically cleave mRNA transcripts to thereby inhibit translation of the protein encoded by the mRNA. Methods of designing and producing ribozymes are known in the art (see, e.g., Scanlon, 1999, Therapeutic Applications of Ribozymes, Humana Press). A ribozyme having specificity for a hair cell differentiation-suppressing mRNA can be designed based upon the nucleotide sequence of a hair cell differentiation-suppressing cDNA (e.g., any of the exemplary cDNA sequences described herein). For example, a derivative of a Tetrahymena L-19 IVS RNA can be constructed in which the nucleotide sequence of the active site is complementary to the nucleotide sequence to be cleaved in a hair cell differentiation-suppressing mRNA (Cech et al. U.S. Pat. No. 4,987,071; and Cech et al., U.S. Pat. No. 5,116,742). Alternatively, an mRNA encoding a hair cell differentiation-suppressing protein can be used to select a catalytic RNA having a specific ribonuclease activity from a pool of RNA molecules (See, e.g., Bartel and Szostak, Science, 261:1411-1418, 1993).

In some embodiments, the administration of the single AAV vector including a sequence that encodes an inhibitory nucleic acid results in at least a 1% to about 99% decrease (e.g., a 1% decrease to about a 99% decrease, a 1% decrease to about a 95% decrease, a 1% decrease to about a 90% decrease, a 1% decrease to about a 85% decrease, a 1% decrease to about a 80% decrease, a 1% decrease to about a 75% decrease, a 1% decrease to about a 70% decrease, a 1% decrease to about a 65% decrease, a 1% decrease to about a 60% decrease, a 1% decrease to about a 55% decrease, a 1% decrease to about a 50% decrease, a 1% decrease to about a 45% decrease, a 1% decrease to about a 40% decrease, a 1% decrease to about a 35% decrease, a 1% decrease to about a 30% decrease, a 1% decrease to about a 25% decrease, a 1% decrease to about a 20% decrease, a 1% decrease to about a 15% decrease, a 1% decrease to about a 10% decrease, about a 20% decrease to about a 99% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 50% decrease to about a 99% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 70% decrease to about a 99% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 80% decrease to about a 99% decrease, about a 80% decrease to about a 95% decrease, about a 80% decrease to about a 90% decrease, about a 80% decrease to about a 85% decrease, about a 90% decrease to about a 99% decrease, or about a 90% decrease to about a 95% decrease) in the level of expression of the hair cell differentiation-suppressing mRNA or protein in a primate cell (e.g., as compared to the level of expression before administration of the single AAV vector that encodes the inhibitory nucleic acid that targets the hair cell differentiation-suppressing mRNA).

Primate Cells

Also provided herein is a cell (e.g., a primate cell, e.g., a hair cell or a supporting cell of the inner ear) that includes any of the nucleic acids, vectors (e.g., at least two different vectors described herein), or compositions described herein. In some embodiments, the primate cell is a human cell (e.g., a human supporting cell or a human hair cell of the inner ear). In other embodiments, the primate is a non-human primate (e.g., simian cell (e.g., a monkey cell (e.g., a marmoset cell, a baboon cell, a macaque cell), or an ape cell (e.g., a gorilla cell, a gibbon cell, an orangutan cell, a chimpanzee cell). Skilled practitioners will appreciate that the AAV vectors described herein can be introduced into any primate cell (e.g., a primate supporting cell or a primate hair cell of the inner ear). Non-limiting examples of AAV vectors and methods for introducing AAV vectors into primate cells are described herein.

In some embodiments, the primate cell can be a supporting hair cell of the inner ear of a mammal. For example, a supporting cell can be Hensen's cells, Deiters' cells, inner pillar cells, outer pillar cells, Claudius cells, inner border cells, inner phalangeal cells, or cells of the stria vascularis.

In some embodiments, the primate cell is a specialized cell of the cochlea. In some embodiments, the primate cell is a hair cell. In some embodiments, the primate cell is a cochlear inner hair cell or a cochlear outer hair cell. In some embodiments, the primate cell is a cochlear inner hair cell. In some embodiments, the primate cell is a cochlear outer hair cell.

In some embodiments, the primate cell is in vitro. In some embodiments, the primate cell is present in a primate. In some embodiments, the primate cell is autologous cell obtained from a primate and cultured ex vivo.

Methods

Also provided herein are methods of promoting differentiation of a supporting cell of an inner ear of a primate into a hair cell that include: administering to the inner ear of the primate a therapeutically effective amount of any of the compositions described herein, where the administering promotes differentiation of the supporting cell of the inner ear of the primate into a hair cell. Differentiation of a supporting cell of the inner ear into a hair cell can be determined using, e.g., indirect functional assays (e.g., hearing testing, e.g., pure tone audiometry).

Also provided herein are methods of increasing the expression level of a hair cell differentiation protein in a hair cell or a supporting cell of an inner ear of a primate that include: administering to the inner ear of the primate a therapeutically effective amount of any of the compositions described herein, where the administering results in an increase (e.g., a 1% to 500% increase, a 1% to 450% increase, a 1% to 400% increase, a 1% to 350% increase, a 1% to 300% increase, a 1% to 250% increase, a 1% to 200% increase, a 1% to 150% increase, a 1% to 100% increase, a 1% to 50% increase, a 50% to 500% increase, a 50% to 450% increase, a 50% to 400% increase, a 50% to 350% increase, a 50% to 300% increase, a 50% to 250% increase, a 50% to 200% increase, a 50% to 150% increase, or a 50% to 100% increase) in the expression level of the hair cell differentiation protein in the hair cell or the supporting cell of the inner ear of the primate (e.g., as compared to the level of expression of the hair cell differentiation protein in the hair cell or the supporting cell of the inner ear of the primate before administration of the composition).

Also provided herein are methods of decreasing the expression level of a hair cell differentiation-suppressing protein in a hair cell or a supporting cell of an inner ear of a primate that include: administering to the inner ear of the primate a therapeutically effective amount of any of the compositions described herein, where the administering results in a decrease (e.g., a 1% decrease to 99% decrease, or any of the subranges of this range described herein) in the expression level of the hair cell differentiation-suppressing protein in the hair cell or the supporting cell of the inner ear of the primate (e.g., as compared to the level of expression of the hair cell differentiation-suppressing protein in the hair cell or the supporting cell of the inner ear of the primate before administration of the composition).

Also provided herein are methods of increasing (e.g., a 1% to 500% increase, or any of the subranges of this range described herein) the number of functional hair cells in a primate in need thereof (e.g., as compared to the number of functional hair cells in a primate prior to the administration of the composition) that include: administering to the inner ear of the primate a therapeutically effective amount of any of the compositions described herein.

Also provided herein are methods of improving hearing in a primate in need thereof, the method comprising administering to the inner ear of the primate a therapeutically effective amount of any of the compositions described herein. In some embodiments, the administering improves hearing in a primate following environmental damage (e.g., noise, chemotherapeutic treatment (e.g., cisplatin treatment) or aminoglycoside treatment).

Also provided herein are methods of repairing a hair cell toxicity-inducing mutation in an endogenous hair cell differentiation gene locus in a hair cell or a supporting cell of an inner ear of a primate that include: administering to the inner ear of the primate a therapeutically effective amount of any of the compositions described herein, where the administering results in repair of the hair cell toxicity-inducing mutation in the endogenous hair cell differentiation gene locus in the hair cell or the supporting cell of the inner ear of the primate.

Also provided herein are methods of decreasing the risk of hearing loss due to hair cell loss or dysfunction in a primate in need thereof that include: administering to the inner ear of the primate a therapeutically effective amount of any of the compositions described herein.

In some embodiments of any of these methods, the primate has been previously identified as having a defective hair cell differentiation gene (e.g., a hair cell differentiation gene having a mutation that results in a decrease in the expression and/or activity of a hair cell differentiation protein encoded by the gene). In some embodiments of any of these methods, the primate has been previously identified as having a defective hair cell differentiation-suppressing gene (e.g., a hair cell differentiation-suppressing gene having a mutation that results in an increase in the expression and/or activity of a hair cell differentiation-suppressing protein encoded by the gene). Some embodiments of any of these methods further include, prior to the introducing or administering step, determining that the primate has a defective hair cell differentiation gene and/or a defective hair cell differentiation-suppressing gene. Some embodiments of any of these methods can further include detecting a mutation in a hair cell differentiation gene and/or a hair cell differentiation-suppressing gene in a primate. Some embodiments of any of the methods can further include identifying or diagnosing a primate as having non-syndromic sensorineural hearing loss. Some embodiments of any of the methods can further include identifying or diagnosing a primate as having syndromic sensorineural hearing loss.

In some embodiments of any of these methods, two or more doses of any of the compositions described herein are introduced or administered into the cochlea of the primate. Some embodiments of any of these methods can include introducing or administering a first dose of the composition into the cochlea of the primate, assessing hearing function of the primate following the introducing or the administering of the first dose, and administering an additional dose of the composition into the cochlea of the primate found not to have a hearing function within a normal range (e.g., as determined using any test for hearing known in the art).

In some embodiments of any of the methods described herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, intra-cochlear administration can be performed using any of the methods described herein or known in the art. For example, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a currette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some embodiments of any of the methods described herein, the primate has or is at risk of developing non-syndromic sensorineural hearing loss. In some embodiments of any of the methods described herein, the primate has been previously identified as having a mutation in a hair cell differentiation gene and/or a hair cell differentiation-suppressing gene. In some embodiments of any of the methods described herein, the primate has any of the mutations in a hair cell differentiation gene and/or a hair cell differentiation-suppressing gene that are described herein or are known in the art to be associated with non-syndromic sensorineural hearing loss or syndromic sensorineural hearing loss.

In some embodiments of any of the methods described herein, the primate has been identified as being a carrier of a mutation in a hair cell differentiation gene and/or a hair cell differentiation-suppressing gene (e.g., via genetic testing). In some embodiments of any of the methods described herein, the primate has been identified as having a mutation in a hair cell differentiation gene and/or a hair cell differentiation-suppressing gene and has been diagnosed with non-syndromic sensorineural hearing loss. In some embodiments of any of the methods described herein, the primate has been identified as having a mutation in a hair cell differentiation gene and/or a hair cell differentiation-suppressing gene and has been diagnosed with syndromic sensorineural hearing loss. In some embodiments of any of the methods described herein, the primate has been identified as having non-syndromic sensorineural hearing loss. In some embodiments of any of the methods described herein, the primate has been identified as having syndromic sensorineural hearing loss.

In some embodiments, successful treatment of non-syndromic sensorineural hearing loss, or syndromic sensorineural hearing loss, can be determined in a primate using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some embodiments of these methods, the primate cell is in vitro. In some embodiments of these methods, the primate cell is originally obtained from a primate and is cultured ex vivo. In some embodiments, the primate cell has previously been determined to have a defective hair cell differentiation protein and/or a defective hair cell differentiation-suppressing protein.

Methods for introducing any of the compositions described herein into a primate cell are known in the art (e.g., via lipofection or through the use of a viral vector, e.g., any of the viral vectors described herein).

An increase in expression of an active hair cell differentiation protein and/or an active hair cell differentiation-suppressing protein (e.g., a full-length hair cell differentiation protein and/or a full-length hair cell differentiation-suppressing protein) as described herein is, e.g., as compared to a control or to the level of expression of an active hair cell differentiation protein and/or a hair cell differentiation-suppressing protein (e.g., a full-length hair cell differentiation protein and/or a full-length hair cell differentiation-suppressing protein) prior to the introduction of the vector(s).

Methods of detecting expression and/or activity of a hair cell differentiation protein and/or a hair cell differentiation-suppressing protein are known in the art. In some embodiments, the level of expression of a hair cell differentiation protein and/or a hair cell differentiation-suppressing protein can be detected directly (e.g., detecting hair cell differentiation protein and/or a hair cell differentiation-suppressing protein or detecting hair cell differentiation mRNA and/or a hair cell differentiation-suppressing mRNA). Non-limiting examples of techniques that can be used to detect expression and/or activity of hair cell differentiation proteins and/or hair cell differentiation-suppressing proteins directly include: real-time PCR, Western blotting, immunoprecipitation, immunohistochemistry, or immunofluorescence. In some embodiments, expression of a hair cell differentiation protein and/or a hair cell differentiation-suppressing protein can be detected indirectly (e.g., through functional hearing tests).

Pharmaceutical Compositions and Kits

In some embodiments, any of the compositions described herein can further include one or more agents that promote the entry of any of the AAV vectors described herein into a primate cell (e.g., a liposome or cationic lipid).

In some embodiments, any of the AAV vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering nucleic acid in vivo into a primate cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007).

Any of the compositions described herein can be, e.g., a pharmaceutical composition. A pharmaceutical composition can include any of the compositions described herein and one or more pharmaceutically or physiologically acceptable carriers, diluents, or excipients. Such compositions may comprise one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives.

In some embodiments, the composition includes a pharmaceutically acceptable carrier (e.g., phosphate buffered saline, saline, or bacteriostatic water). Upon formulation, solutions will be administered in a manner compatible with the dosage formulation and in such amount as is therapeutically effective. The formulations are easily administered in a variety of dosage forms such as injectable solutions, injectable gels, drug-release capsules, and the like.

As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein.

In some embodiments, a single dose of any of the compositions described herein can include a total amount (e.g., total sum amount of the at least two different AAV vectors, or the total amount of the single AAV vector) of at least 1 ng, at least 2 ng, at least 4 ng, about 6 ng, about 8 ng, at least 10 ng, at least 20 ng, at least 30 ng, at least 40 ng, at least 50 ng, at least 60 ng, at least 70 ng, at least 80 ng, at least 90 ng, at least 100 ng, at least 200 ng, at least 300 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μs, at least 20 μg, at least 22 μg, at least 24 μg, at least 26 μg, at least 28 μg, at least 30 μg at least 32 μg, at least 34 μg, at least 36 μg, at least 38 μg, at least 40 μg, at least 42 μg, at least 44 μg, at least 46 μg, at least 48 fig, at least 50 μg, at least 52 μg, at least 54 μg, at least 56 μg, at least 58 μg, at least 60 μg, at least 62 μg, at least 64 μg, at least 66 μg, at least 68 μg, at least 70 μg, at least 72 μg, at least 74 μg, at least 76 μg, at least 78 μg, at least 80 μg, at least 82 μg, at least 84 μg, at least 86 μg, at least 88 μg, at least 90 μg, at least 92 μg, at least 94 μg, at least 96 μg, at least 98 μg, at least 100 μg, at least 102 μg, at least 104 μg, at least 106 μg, at least 108 μg, at least 110 μg, at least 112 μg, at least 114 μg, at least 116 μg, at least 118 μg, at least 120 μg, at least 122 μg, at least 124 μg, at least 126 μg, at least 128 μg, at least 130 μg at least 132 μg, at least 134 μg, at least 136 μg, at least 138 μg, at least 140 μg, at least 142 μg, at least 144 μg, at least 146 μg, at least 148 μg, at least 150 μg, at least 152 μg, at least 154 μg, at least 156 μg, at least 158 μg, at least 160 μg, at least 162 μg, at least 164 μg, at least 166 μg, at least 168 μg, at least 170 μg, at least 172 μg, at least 174 μg, at least 176 μg, at least 178 μg, at least 180 μg, at least 182 mg, at least 184 μg, at least 186 μg, at least 188 μg, at least 190 μg, at least 192 μg, at least 194 μg, at least 196 μg, at least 198 μg, or at least 200 μg, e.g., in a buffered solution.

The compositions provided herein can be, e.g., formulated to be compatible with their intended route of administration. A non-limiting example of an intended route of administration is local administration (e.g., intra-cochlear administration). In some embodiments, the therapeutic compositions are formulated to include a lipid nanoparticle. In some embodiments, the therapeutic compositions are formulated to include a polymeric nanoparticle. In some embodiments, the therapeutic compositions are formulated to comprise a synthetic perilymph solution. An exemplary synthetic perilymph solution includes 20-200 mM NaCl; 1-5 mM KCl; 0.1-10 mM CaCl₂; 1-10 mM glucose; 2-50 mM HEPES, having a pH of between about 6 and about 9.

Also provided are kits including any of the compositions described herein. In some embodiments, a kit can include a solid composition (e.g., a lyophilized composition including the single AAV vector or the at least two different vectors described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

Devices and Surgical Methods

Provided herein are therapeutic delivery systems for treating non-syndromic sensorineural hearing loss, or syndromic sensorineural hearing loss. In one aspect, the therapeutic delivery systems include i) a medical device capable of creating one or a plurality of incisions in a round window membrane of an inner ear of a primate in need thereof, and ii) an effective dose of a composition (e.g., any of the compositions described herein). In some embodiments, the medical device includes a plurality of micro-needles.

Also provided herein are surgical methods for treatment of hearing loss (e.g., non-syndromic sensorineural hearing loss, syndromic sensorineural hearing loss). In some embodiments, the methods include the steps of: introducing into a cochlea of a primate first incision at a first incision point; and administering intra-cochlearly a therapeutically effective amount of any of the compositions provided herein. In some embodiments, the composition is administered to the primate at the first incision point. In some embodiments, the composition is administered to the primate into or through the first incision.

In some embodiments of any of the methods described herein, any of the compositions described herein is administered to the primate into or through the cochlea oval window membrane. In some embodiments of any of the methods described herein, any of the compositions described herein is administered to the primate into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations that become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### EXAMPLES

### Example 1. AAV Single Vector Injection into the Inner Ear

Immunofluorescent staining was performed on cochlear tissue of a cynomolgus macaque (non-human primate) following administration of a single Anc80-GFP AAV vector directly into the inner ear through the round window.

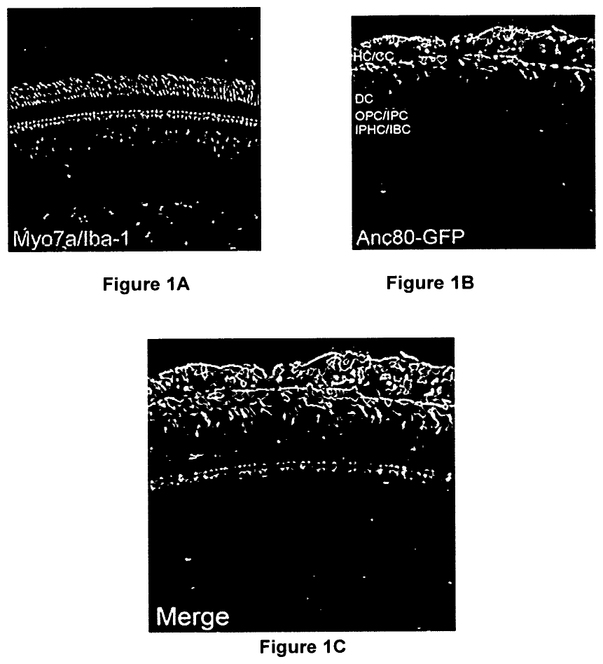

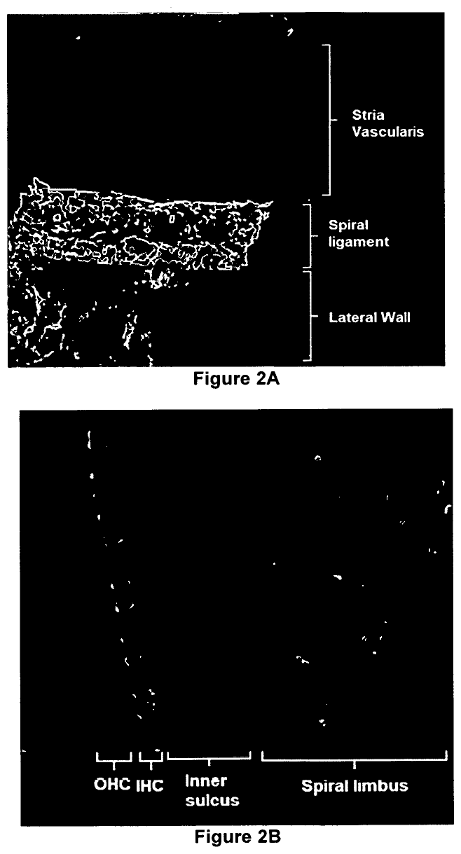

The cochlear tissue from the treated macaque was processed for immunofluorescence analysis using Myo7a as a marker for hair cells and Iba-1 as a marker for macrophages. The middle turn is representative of the entire sensory epithelium. The data in FIGS. 1A-1C show clear GFP expression in both the hair cells and the supporting cells, including the following supporting cell subtypes: Hensen's cells (HC), Claudius cells (CC), Dieter cells (DC), inner and outer pillar cells (OPC/IPC), inner border cells, and inner phalangeal cells (IPHC/IBC). These data demonstrate successful Anc80-GFP AAV vector transduction into different cell types of the inner ear sensory epithelium, and the resulting expression of the encoded reporter gene (GFP) in these different cell types. These data indicate the present claimed compositions including a single AAV vector or two or more AAV vectors can be used to express a gene in hairs cells and supporting cells, and can be used to repair a mutation in a gene in hair cells and supporting cells. FIGS. 2A and 2B are representative images of Anc80-GFP immunofluorescent staining of the cochlear tissue. As shown in FIG. 2B, expression is detected in inner hair cells.

### Example 2. Exemplary Vectors for Promoting Differentiation of a Supporting Cell of an Inner Ear of a Primate into a Hair Cell

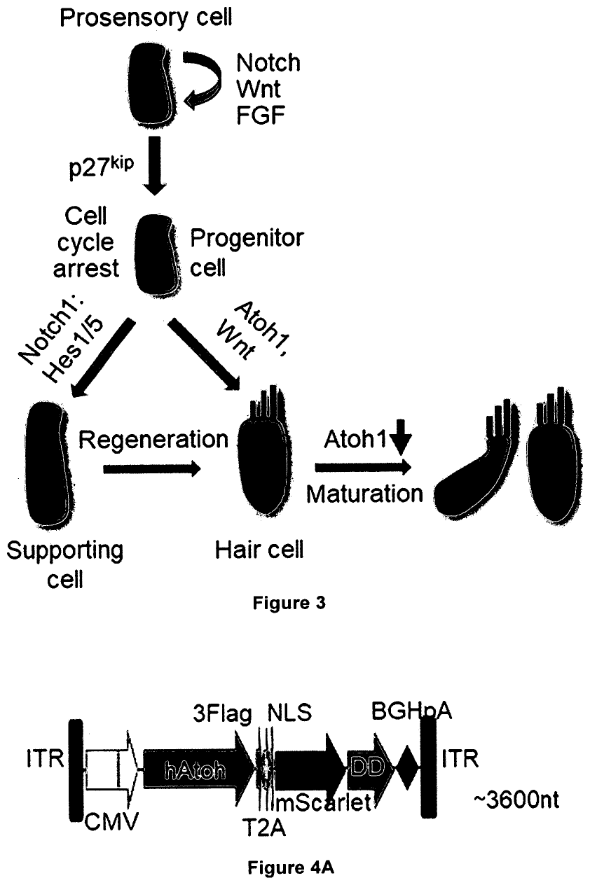

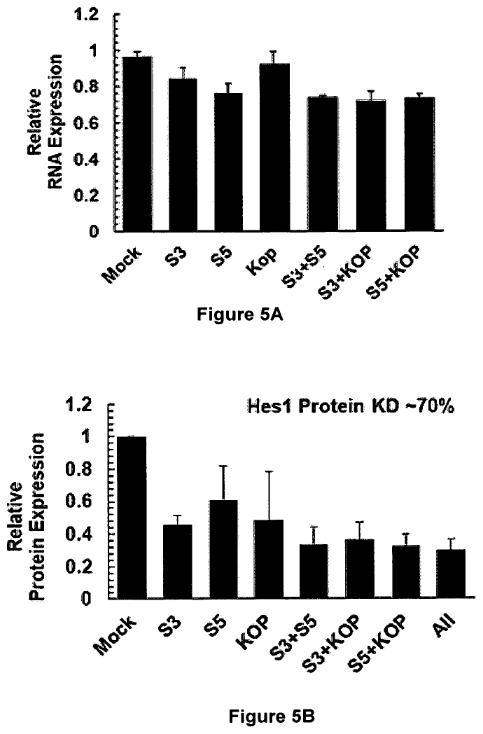

As shown in FIG. 3, progenitor cells differentiate into either supporting cells or hair cells. Expression of Notch 1 and Hes1/5 in progenitor cells leads to the generation of supporting cells, whereas expression of Atoh1 and Wnt in progenitor cells leads to the generation of hair cells. FIGS. 4A-4C are exemplary vectors that can be used to promote differentiation of a supporting cell. FIG. 4D is an exemplary vector that encodes a shRNA that decreases the expression of a hair cell differentiation-suppressing protein in a primate cell. The data in FIG. 5A shows the relative mRNA expression levels of Hes1 in HEK293 cells that were transfected with a vector encoding S3 (SEQ ID NO: 68), a vector encoding S5 (SEQ ID NO: XX), a vector encoding Kop (SEQ ID NO: 75), vectors encoding S3 plus S5, vectors encoding S3 plus Kop and vectors encoding S5 and Kop. Relative expression was determined using RTqPCR. Cells transfected with the dual vectors show increased reduction in Hes1 mRNA levels. The data in FIG. 5B shows reduced Hes1 protein levels in these same cells as determined by Western blotting. Taken together, the data in FIGS. 5A and 5B confirms the ability of vectors to decrease target mRNA and protein levels.

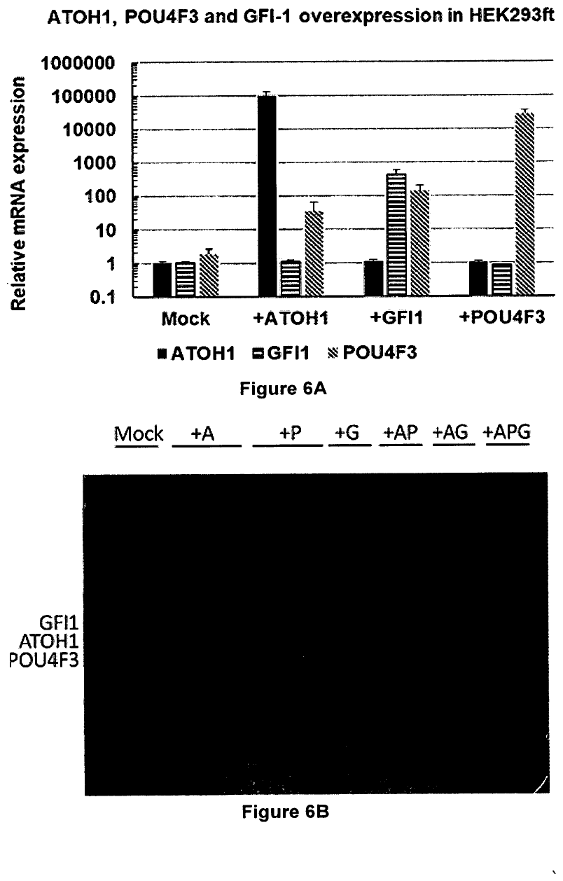

The data in FIGS. 6A and 6B show overexpression of ATOH1, POU4F3 and GFI-1 in HEK293FT cells that were transfected with the vectors of FIGS. 4A-4D. As shown in FIG. 6A, overexpression of POU4F3 in HEK293FT cells also led to an increase in ATOH1 and GFI-1 mRNA levels. FIG. 6B shows overexpression of ATOH1, GFI-1 and POU4F3 in HEK293FT cells, respectively.

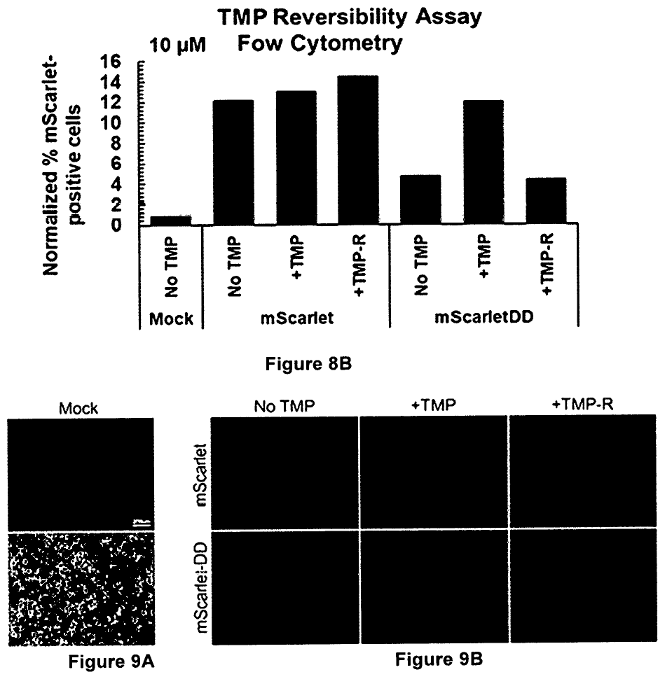

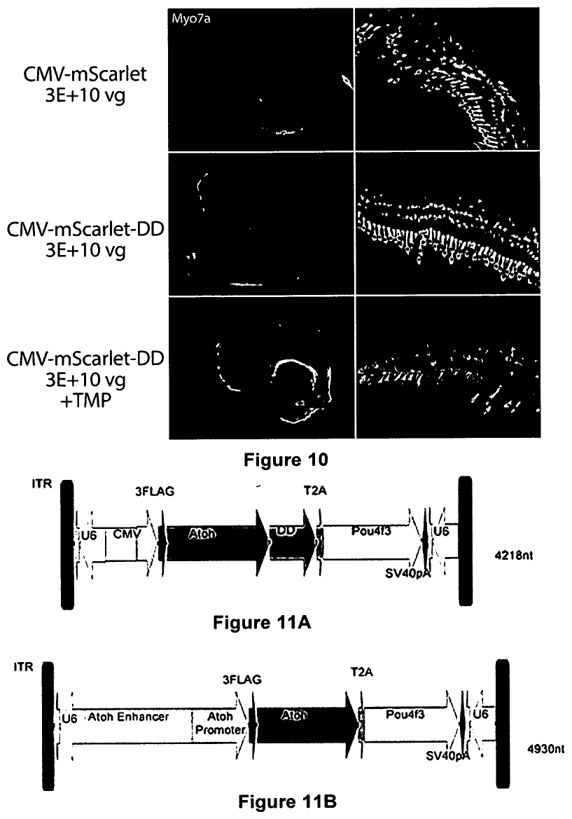

HEK293FT cells were transfected with mScarlet and mScarlet-DD vectors (FIGS. 7A-7B). The data in FIGS. 8A and 8B show the functionality and reversibility of the destabilizing domain (DD) using fluorescence microscopy and flow cytometry, respectively. As shown in FIG. 8A, the percentage of mScarlet positive cells increased proportionately with increasing concentration of TMP in mScarlet-DD transfected HEK293FT cells, whereas the percentage of mScarlet positive cells remained constant regardless of TMP concentration in mScarlet transfected HEK293FT cells. As shown in FIGS. 9A and 9B, mScarlet expression was seen in all HEK293FT cells transfected with mScarlet, whereas mScarlet expression was primarily seen in mScarlet-DD transfected cells in the presence of TMP. FIG. 10 displays the same response in cochlear explants, where transduction and subsequent expression of mScarlet is seen in hair cells and supporting cells, whereas expression of mScarlet-DD is only seen in the presence of TMP.

FIGS. 11A and 11B are exemplary combined vectors that can be used to promoter differentiation of a supporting cells. The vectors are combined from vectors of FIGS. 4A-C.

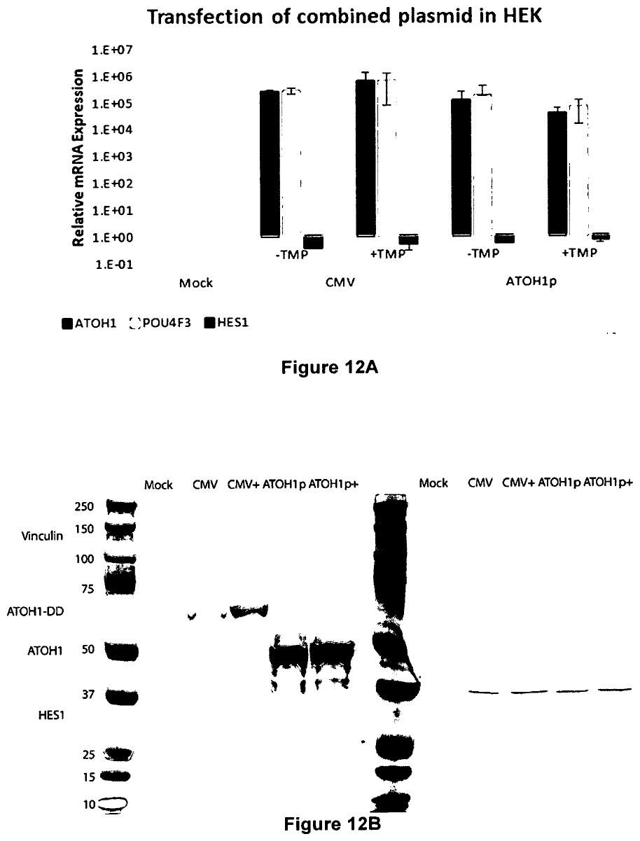

The data in FIGS. 12A and 12B show overexpression of ATOH1 and POU4F3 and reduction in HES1 mRNA and protein respectively, after transfection with the vectors of FIGS. 11A and 11B.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1-40. (canceled)

41. A method of promoting differentiation of a supporting cell of an inner ear of a primate into a hair cell, the method comprising:
administering to the inner ear of the primate an adeno-associated virus (AAV) vector, where the AAV vector comprises a nucleic acid sequence encoding a hair cell differentiation protein,
wherein the administering promotes differentiation of the supporting cell of the inner ear of the primate into a hair cell.

42-51. (canceled)

52. The method of claim 41, wherein the hair cell differentiation protein is selected from the group consisting of: atonal bHLH transcription factor 1 (ATOH1), POU Class 4 Homeobox 3 (POU4F3), catenin beta 1 (CTNNB1), Noggin (NOG), growth factor independent 1 transcriptional repressor (GFI-1), neurotrophin 3 (NTF3) and brain-derived neurotrophic factor (BDNF).

53. The method of claim 41, wherein the AAV vector is encapsulated by an AAV capsid.

54. The method of claim 53, wherein the AAV capsid is an Anc80 capsid.

55. The method of claim 41, wherein the primate has a defective hair cell differentiation gene prior to administration.

56. The method of claim 41, wherein the subject has non-syndromic sensorineural hearing loss or syndromic sensorineural hearing loss.

57. A method of promoting differentiation of a supporting cell of an inner ear of a primate into a hair cell, the method comprising:
administering to the inner ear of the primate two different adeno-associated virus (AAV) vectors, wherein:
a first AAV vector comprises a first nucleic acid sequence that encodes a first hair cell differentiation protein; and
a second AAV vector comprises a second nucleic acid sequence that encodes a second hair cell differentiation protein, wherein the first hair cell differentiation protein and the second hair cell differentiation protein are different proteins,
wherein the administering promotes differentiation of the supporting cell of the inner ear of the primate into a hair cell.

58. The method of claim 57, wherein the first and the second hair cell differentiation proteins are selected from the group consisting of: atonal bHLH transcription factor 1 (ATOH1), POU Class 4 Homeobox 3 (POU4F3), catenin beta 1 (CTNNB1), Noggin (NOG), growth factor independent 1 transcriptional repressor (GFI-1), neurotrophin 3 (NTF3) and brain-derived neurotrophic factor (BDNF).

59. The method of claim 57, wherein the first AAV vector is encapsulated by a first AAV capsid and the second AAV vector is encapsulated by a second AAV capsid.

60. The method of claim 59, wherein the first and second AAV capsids are Anc80 capsids.

61. The method of claim 57, wherein the primate has a defective hair cell differentiation gene prior to administration.

62. The method of claim 57, wherein the subject has non-syndromic sensorineural hearing loss or syndromic sensorineural hearing loss.

63. A method of promoting differentiation of a supporting cell of an inner ear of a primate into a hair cell, the method comprising:
administering to the inner ear of the primate at least one adeno-associated virus (AAV) vector that comprises an inhibitory nucleic acid that decreases the expression of a hair cell differentiation-suppressing protein in a primate cell, wherein the administering promotes differentiation of the supporting cell of the inner ear of the primate into a hair cell.

64. The method of claim 63, wherein the inhibitory nucleic acid is a short interfering RNA (siRNA), a short hairpin RNA (shRNA), an antisense oligonucleotide, or a ribozyme.

65. The method of claim 63, wherein the hair cell differentiation-suppressing gene is hes family bHLH transcription factor 1 (HES1), hes family bHLH transcription factor 5 (HES5), sex determining region Y-box 2 (SOX2), and p27kip (CDKN1B).

66. The method of claim 63, wherein the at least one AAV vector is encapsulated by an AAV capsid.

67. The method of claim 66, wherein the AAV capsid is an Anc80 capsid.

68. The method of claim 63, wherein the primate has a defective hair cell differentiation gene prior to administration.

69. The method of claim 63, wherein the subject has non-syndromic sensorineural hearing loss or syndromic sensorineural hearing loss.
